(19) United States
(12) Patent Application Publication
(10) Pub. No.: US 2024/0270813 A1
(43) Pub. Date: Aug. 15, 2024

(54) T CELL RECEPTORS (TCRs) TARGETING MINOR HISTOCOMPATIBILITY ANTIGEN HA-1

(71) Applicants: University Of Pittsburgh - Of The Commonwealth System Of Higher Education, Pittsburgh, PA (US); BlueSphere Bio, Inc., Pittsburgh, PA (US)

(72) Inventors: Sawa Ito, Pittsburgh, PA (US); Warren David Shlomchik, Pittsburgh, PA (US); Mark Jay Shlomchik, Pittsburgh, PA (US); Constantinos George Panousis, Pittsburgh, PA (US); Josh Kim, Pittsburgh, PA (US); Erik Martin, Pittsburgh, PA (US); Daniel Wikenheiser, Pittsburgh, PA (US)

(21) Appl. No.: 18/557,647

(22) PCT Filed: May 2, 2022
(86) PCT No.: PCT/US2022/027311

§ 371 Date: Oct. 27, 2023

Related U.S. Application Data
(60) Provisional application No. 63/183,515, filed on May 3, 2021.
(60) Provisional application No. 63/251,261, filed on Oct. 1, 2021.

(51) Int. Cl. C07K 14/725; A61P 35/00; C12N 5/0783; C12N 15/63

(52) U.S. Cl. CPC C07K 14/7051; A61P 35/00; C12N 5/0636; C12N 15/63; C07K 2317/565; C12N 2510/00

(57) ABSTRACT

Provided herein are T cell receptors (TCRs) or antigen-binding fragments thereof, such as those that recognize or bind a hematopoictically-restricted minor histocompatibility antigen, e.g., HA-1. In particular, the present disclosure relates to TCRs that bind or recognize particular HA-1 peptides in the context of a major histocompatibility complex (MHC) molecule. The present disclosure further relates to nucleic acids encoding such TCRs, engineered cells comprising such TCRs, methods of isolating such TCRs, and uses thereof, for example, in cell therapy.

### CROSS-REFERENCE TO RELATED APPLICATIONS

This application claims the benefit of U.S. Provisional Application No. 63/183,515, filed May 3, 2021, and U.S. Provisional Application No. 63/251,261, filed Oct. 1, 2021, each of which is incorporated herein by reference.

### SEQUENCE LISTING

The Sequence Listing written in file 578238_SeqListing_ST25. txt is 709 kilobytes in size, was created Apr. 28, 2022, and is hereby incorporated by reference.

### FIELD

The present disclosure relates in some aspects to T cell receptors (TCRs) or antigen-binding fragments thereof, such as those that recognize or bind a hematopoietically-restricted minor histocompatibility antigen, e.g., HA-1. In particular, the present disclosure relates to TCRs that bind to or recognize particular HA-1 peptides in the context of a major histocompatibility complex (MHC) molecule. The present disclosure further relates to nucleic acids encoding such TCRs, engineered cells comprising such TCRs, methods of isolating such TCRs and uses thereof, for example, in cell therapy.

### BACKGROUND

Allogeneic hematopoietic stem cell transplantation (alloSCT) may be used for treatment of diseases or conditions such as hematologic malignancies and other nonmalignant conditions. Some subjects may relapse after alloSCT. Improved treatments are necessary to attain an optimal treatment outcome. Provided are embodiments that meet such needs.

### SUMMARY

Provided herein are TCRs or antigen-binding fragment thereof, comprising: an alpha chain comprising a variable alpha (Vα) region and a beta chain comprising a variable beta (Vβ) region; or a gamma chain comprising a variable gamma (Vγ) region and a delta chain comprising a variable delta (Vδ) region; wherein: the Vα or Vγ region comprises a complementarity determining region 3 (CDR-3) comprising SEQ ID NO:3, and the Vβ or Vδ region comprises a CDR-3 comprising SEQ ID NO:11; the Vα or Vγ region comprises a CDR-3 comprising SEQ ID NO:21, and the Vβ or Vδ region comprises a CDR-3 comprising SEQ ID NO:27; the Vα or Vγ region comprises a CDR-3 comprising SEQ ID NO:37, and the Vβ or Vδ region comprises a CDR-3 comprising SEQ ID NO:43; the Vα or Vγ region comprises a CDR-3 comprising SEQ ID NO:51, and the Vβ or Vδ region comprises a CDR-3 comprising SEQ ID NO:57; the Vα or Vγ region comprises a CDR-3 comprising SEQ ID NO:65, and the Vβ or Vδ region comprises a CDR-3 comprising SEQ ID NO:57; the Vα or Vγ region comprises a CDR-3 comprising SEQ ID NO:78, and the Vβ or Vδ region comprises a CDR-3 comprising SEQ ID NO:84; the Vα or Vγ region comprises a CDR-3 comprising SEQ ID NO:92, and the Vβ or Vδ region comprises a CDR-3 comprising SEQ ID NO:98; the Vα or Vγ region comprises a CDR-3 comprising SEQ ID NO: 106, and the Vβ or Vδ region comprises a CDR-3 comprising SEQ ID NO: 112; the Vα or Vγ region comprises a CDR-3 comprising SEQ ID NO:120, and the Vβ or Vδ region comprises a CDR-3 comprising SEQ ID NO: 126; the Vα or Vγ region comprises a CDR-3 comprising SEQ ID NO:136, and the Vβ or Vδ region comprises a CDR-3 comprising SEQ ID NO:142; the Vα or Vγ region comprises a CDR-3 comprising SEQ ID NO: 152, and the Vβ or Vδ region comprises a CDR-3 comprising SEQ ID NO:158; the Vα or Vγ region comprises a CDR-3 comprising SEQ ID NO:166, and the Vβ or Vδ region comprises a CDR-3 comprising SEQ ID NO: 172; the Vα or Vγ region comprises a CDR-3 comprising SEQ ID NO:180, and the Vβ or Vδ region comprises a CDR-3 comprising SEQ ID NO:186; the Vα or Vγ region comprises a CDR-3 comprising SEQ ID NO:194, and the Vβ or Vδ region comprises a CDR-3 comprising SEQ ID NO:200; the Vα or Vγ region comprises a CDR-3 comprising SEQ ID NO:208, and the Vβ or Vδ region comprises a CDR-3 comprising SEQ ID NO:214; the Vα or Vγ region comprises a CDR-3 comprising SEQ ID NO:224, and the Vβ or Vδ region comprises a CDR-3 comprising SEQ ID NO:230; the Vα or Vγ region comprises a CDR-3 comprising SEQ ID NO:238, and the Vβ or Vδ region comprises a CDR-3 comprising SEQ ID NO:244; the Vα or Vγ region comprises a CDR-3 comprising SEQ ID NO:252, and the Vβ or Vδ region comprises a CDR-3 comprising SEQ ID NO:258; the Vα or Vγ region comprises a CDR-3 comprising SEQ ID NO:268, and the Vβ or Vδ region comprises a CDR-3 comprising SEQ ID NO: 158; the Vα or Vγ region comprises a CDR-3 comprising SEQ ID NO:278, and the Vβ or Vδ region comprises a CDR-3 comprising SEQ ID NO:284; the Vα or Vγ region comprises a CDR-3 comprising SEQ ID NO:359, and the Vβ or Vδ region comprises a CDR-3 comprising SEQ ID NO:363; the Vα or Vγ region comprises a CDR-3 comprising SEQ ID NO:369, and the Vβ or Vo region comprises a CDR-3 comprising SEQ ID NO:373; the Vα or Vγ region comprises a CDR-3 comprising SEQ ID NO:379, and the Vβ or Vδ region comprises a CDR-3 comprising SEQ ID NO:383; the Vα or Vγ region comprises a CDR-3 comprising SEQ ID NO:389, and the Vβ or Vδ region comprises a CDR-3 comprising SEQ ID NO:393; the Vα or Vγ region comprises a CDR-3 comprising SEQ ID NO:399, and the Vβ or Vδ region comprises a CDR-3 comprising SEQ ID NO:403; the Vα or Vγ region comprises a CDR-3 comprising SEQ ID NO:409, and the Vβ or Vδ region comprises a CDR-3 comprising SEQ ID NO:413; the Vα or Vγ region comprises a CDR-3 comprising SEQ ID NO:419, and the Vβ or Vδ region comprises a CDR-3 comprising SEQ ID NO:423; the Vα or Vγ region comprises a CDR-3 comprising SEQ ID NO:429, and the Vβ or Vδ region comprises a CDR-3 comprising SEQ ID NO:433; the Vα or Vγ region comprises a CDR-3 comprising SEQ ID NO:439, and the Vβ or Vδ region comprises a CDR-3 comprising SEQ ID NO:443; the Vα or Vγ region comprises a CDR-3 comprising SEQ ID NO:449, and the Vβ or Vδ region comprises a CDR-3 comprising SEQ ID NO:453; or the Vα or Vγ region comprises a CDR-3 comprising SEQ ID NO:480, and the Vβ or Vδ region comprises a CDR-3 comprising SEQ ID NO:484.

Provided herein are TCRs or antigen-binding fragments thereof, comprising: an alpha chain comprising a variable alpha (Vα) region and a beta chain comprising a variable beta (Vβ) region; or a gamma chain comprising a variable gamma (Vγ) region and a delta chain comprising a variable delta (Vδ) region; wherein: the Vα or Vγ region comprises a complementarity determining region 3 (CDR-3) comprising AVRXAXBRTSGTYKYI (SEQ ID NO:476), and the Vβ or Vδ region comprises a CDR-3 comprising ASSXCXDXEGXFXGQF (SEQ ID NO:478), wherein each of X_{A}, X_{B}, X_{C}, X_{D}, X_{E}, X_{F}, X_{G} can by any amino acid. In some embodiments, X_{A} can be an aliphatic amino acid (i.e., Ala, Ile, Leu, Val, or Pro), glycine, a sulfur-containing amino acid (i.e., Cys or Met), a hydroxyl amino acid (i.e., Ser or Thr), or an acidic residue (i.e., Asp or Glu). In some embodiments, X_{A} can be Ala, Gly, Cys, Ser, Thr, Val, Asp, or Glu. In some embodiments, X_{B} can be a cyclic imino group-containing amino acid or a hydroxyl amino acid. In some embodiments, X_{B} can be Pro, Ser, or Thr. In some embodiments, X_{C} can be an aliphatic amino acid or an aromatic amino acid (i.e., Phe, Tyr, or Trp). In some embodiments, X_{C} can be Leu or Phe. In some embodiments, X_{D} can be an aliphatic amino acid. In some embodiments, X_{D} can be Val or Leu. In some embodiments, X_{E} can be a hydroxyl amino acid, an aliphatic amino acid or an amide amino acid (i.e., Asn or Gln). In some embodiments, X_{E} can be Ser, Ala, Gln, or Asn. In some embodiments, X_{F} can be an acidic amino acid or an amide amino acid. In some embodiments, X_{F} can be Glu or Asn. In some embodiments, X_{G} can be an acid amino acid or a hydroxyl amino acid. In some embodiments, X_{G} can be Glu or Thr. The Vα or Vγ region CDR-3 of any of the described TCRs can be substituted for a CDR-3 comprising SEQ ID NO:476. The Vβ or Vo region CDR-3 of any of the described TCRs can be substituted for a CDR-3 comprising SEQ ID NO:478. For any of the described TCRs, the Vα or Vγ region CDR-3 can be substituted for a CDR-3 comprising SEQ ID NO:476 and the Vβ or Vδ region CDR-3 can be substituted for a CDR-3 comprising SEQ ID NO: 478.

Provided herein are TCRs or antigen-binding fragments thereof, comprising: an alpha chain comprising a variable alpha (Vα) region and a beta chain comprising a variable beta (Vβ) region; or a gamma chain comprising a variable gamma (Vγ) region and a delta chain comprising a variable delta (Vδ) region; wherein: the Vα or Vγ region comprises a complementarity determining region 3 (CDR-3) comprising SEQ ID NO:459, 460, 461, 462, 463, 464, 476, or 477, and the Vβ or Vδ region comprises a CDR-3 comprising SEQ ID NO:465, 466, 467, 468, 469, 478, or 479. In some embodiments, the Vα or Vγ region comprises a CDR-3 comprising SEQ ID NO:459, and the Vβ or Vδ region comprises a CDR-3 comprising SEQ ID NO:465, 466, 467, 468, 469, 478, or 479; the Vα or Vγ region comprises a CDR-3 comprising SEQ ID NO:460, and the Vβ or Vδ region comprises a CDR-3 comprising SEQ ID NO:465, 466, 467, 468, 469, 478, or 479; the Vα or Vγ region comprises a CDR-3 comprising SEQ ID NO:461, and the Vβ or Vδ region comprises a CDR-3 comprising SEQ ID NO:465, 466, 467, 468, 469, 478, or 479; the Vα or Vγ region comprises a CDR-3 comprising SEQ ID NO:462, and the Vβ or Vδ region comprises a CDR-3 comprising SEQ ID NO:465, 466, 467, 468, 469, 478, or 479; the Vα or Vγ region comprises a CDR-3 comprising SEQ ID NO:463, and the Vβ or Vδ region comprises a CDR-3 comprising SEQ ID NO:465, 466, 467, 468, 469, 478, or 479; the Vα or Vγ region comprises a CDR-3 comprising SEQ ID NO: 464, and the Vβ or Vδ region comprises a CDR-3 comprising SEQ ID NO:465, 466, 467, 468, 469, 478, or 479; the Vα or Vγ region comprises a CDR-3 comprising SEQ ID NO:459, 460, 461, 462, 463, 464, 476, or 477, and the Vβ or Vδ region comprises a CDR-3 comprising SEQ ID NO:465; the Vα or Vγ region comprises a CDR-3 comprising SEQ ID NO:459, 460, 461, 462, 463, 464, 476, or 477, and the Vβ or Vδ region comprises a CDR-3 comprising SEQ ID NO:466; the Vα or Vγ region comprises a CDR-3 comprising SEQ ID NO:459, 460, 461, 462, 463, 464, 476, or 477, and the Vβ or Vδ region comprises a CDR-3 comprising SEQ ID NO:467; the Vα or Vγ region comprises a CDR-3 comprising SEQ ID NO:459, 460, 461, 462, 463, 464, 476, or 477, and the Vβ or Vo region comprises a CDR-3 comprising SEQ ID NO:468; or the Vα or Vγ region comprises a CDR-3 comprising SEQ ID NO:459, 460, 461, 462, 463, 464, 476, or 477, and the Vβ or Vδ region comprises a CDR-3 comprising SEQ ID NO:469. In some embodiments, the TCR or antigen-binding fragment thereof, the Vα or Vγ region contains a CDR-3 comprising SEQ ID NO:463, and the Vβ or Vδ region contains a CDR-3 comprising SEQ ID NO:469. In some embodiments; the Vα or Vγ region comprises: a complementarity determining region 1 (CDR-1) comprising SEQ ID NO:1, and a complementarity determining region 2 (CDR-2) comprising SEQ ID NO:2; a CDR-1 comprising SEQ ID NO: 19, and a CDR-2 comprising SEQ ID NO: 20; a CDR-1 comprising SEQ ID NO: 35, and a CDR-2 comprising SEQ ID NO: 36; a CDR-1 comprising SEQ ID NO: 76, and a CDR-2 comprising SEQ ID NO: 77; a CDR-1 comprising SEQ ID NO: 134, and a CDR-2 comprising SEQ ID NO: 135; a CDR-1 comprising SEQ ID NO: 150, and a CDR-2 comprising SEQ ID NO: 151; a CDR-1 comprising SEQ ID NO: 222, and a CDR-2 comprising SEQ ID NO: 223; or a CDR-1 comprising SEQ ID NO: 266, and a CDR-2 comprising SEQ ID NO: 267; and the Vβ or Vδ region comprises a CDR-1 comprising SEQ ID NO:9, and a CDR-2 comprising SEQ ID NO: 10. In some embodiments, the TCR or antigen binding fragment thereof provided herein has the ability to bind to a peptide epitope of a minor histocompatibility antigen HA-1 in the context of an MHC molecule. The Vα or Vγ region CDR-3 of any of the described TCRs can be substituted for a CDR-3 comprising SEQ ID NO:459, 460, 461, 462, 463, 464, 476, or 477. The Vβ or Vo region CDR-3 of any of the described TCRs can be substituted for a CDR-3 comprising SEQ ID NO:465, 466, 467, 468, 469, 478, or 479. For any of the described TCRs, the Vα or Vγ region CDR-3 can be substituted for a CDR-3 comprising SEQ ID NO:459, 460, 461, 462, 463, 464, 476, or 477, and the Vβ or Vδ region CDR-3 can be substituted for a CDR-3 comprising SEQ ID NO:465, 466, 467, 468, 469, 478, or 479.

Provided herein are TCRs or antigen-binding fragments thereof, comprising: an alpha chain comprising a Vα region and a beta chain comprising a Vβ region; or a gamma chain comprising a Vγ region and a delta chain comprising a Vδ region; wherein: the Vα or Vγ region comprises a CDR-3 comprising SEQ ID NO:470, and the Vβ or Vδ region comprises a CDR-3 comprising SEQ ID NO:57; the Vα or Vγ region comprises a CDR-3 comprising SEQ ID NO:471, and the Vβ or Vδ region comprises a CDR-3 comprising SEQ ID NO:57; the Vα or Vγ region comprises a CDR-3 comprising SEQ ID NO:472, and the Vβ or Vδ region comprises a CDR-3 comprising SEQ ID NO:57; the Vα or Vγ region comprises a CDR-3 comprising SEQ ID NO:473, and the Vβ or Vδ region comprises a CDR-3 comprising SEQ ID NO:57; or the Vα or Vγ region comprises a CDR-3 comprising SEQ ID NO:474, and the Vβ or Vδ region comprises a CDR-3 comprising SEQ ID NO:475. In some embodiments, the Vα or Vγ region comprises: a complementarity determining region 1 (CDR-1) comprising SEQ ID NO:1, and a complementarity determining region 2 (CDR-2) comprising SEQ ID NO:2; a CDR-1 comprising SEQ ID NO: 19, and a CDR-2 comprising SEQ ID NO: 20; a CDR-1 comprising SEQ ID NO: 35, and a CDR-2 comprising SEQ ID NO: 36; a CDR-1 comprising SEQ ID NO: 76, and a CDR-2 comprising SEQ ID NO: 77; a CDR-1 comprising SEQ ID NO: 134, and a CDR-2 comprising SEQ ID NO: 135; a CDR-1 comprising SEQ ID NO: 150, and a CDR-2 comprising SEQ ID NO: 151; a CDR-1 comprising SEQ ID NO: 222, and a CDR-2 comprising SEQ ID NO: 223; or a CDR-1 comprising SEQ ID NO: 266, and a CDR-2 comprising SEQ ID NO: 267; and the Vβ or Vδ region comprises a CDR-1 comprising SEQ ID NO:9, and a CDR-2 comprising SEQ ID NO:10. In some embodiments, the TCR or antigen binding fragment thereof provided herein has the ability to bind to a peptide epitope of a minor histocompatibility antigen HA-1 in the context of an MHC molecule. In some embodiments, the Vα or Vγ region CDR-3 of TCR D can be substituted for a CDR-3 comprising SEQ ID NO:470, 471, 472, 473, or 474. In some embodiments, the Vβ or Vδ region CDR-3 of TCR D can be substituted for a CDR-3 comprising SEQ ID NO:475. In some embodiments, the Vα or Vγ region CDR-3 of TCR D can be substituted for a CDR-3 comprising SEQ ID NO: 470, 471, 472, 473, or 474, and the Vβ or Vδ region CDR-3 can be substituted for a CDR-3 comprising SEQ ID NO:475.

In some of any of the embodiments provided herein, the Vα or Vγ region comprises a complementarity determining region 1 (CDR-1) comprising SEQ ID NO:1, and a complementarity determining region 2 (CDR-2) comprising SEQ ID NO:2, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO:19, and a CDR-2 comprising SEQ ID NO:20, and the Vβ or Vδ region comprises a CDR-1 comprising SEQ ID NO:9, and a CDR-2 comprising SEQ ID NO:10; the Vα or Vγ region comprises a CDR-1 comprising SEQ ID NO:35, and a CDR-2 comprising SEQ ID NO:36, and the Vβ or Vδ region comprises a CDR-1 comprising SEQ ID NO:9, and a CDR-2 comprising SEQ ID NO:10; the Vα or Vγ region comprises a CDR-1 comprising SEQ ID NO:1, and a CDR-2 comprising SEQ ID NO:2, and the Vβ or Vδ region comprises a CDR-1 comprising SEQ ID NO:9, and a CDR-2 comprising SEQ ID NO:10; the Vα or Vγ region comprises a CDR-1 comprising SEQ ID NO:1, and a CDR-2 comprising SEQ ID NO:2, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO:76, and a CDR-2 comprising SEQ ID NO:77, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO:19, and a CDR-2 comprising SEQ ID NO:20, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO:76, and a CDR-2 comprising SEQ ID NO:77, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO:35, and a CDR-2 comprising SEQ ID NO:36, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO: 134, and a CDR-2 comprising SEQ ID NO:135, and the Vβ or Vδ region comprises a CDR-1 comprising SEQ ID NO:9, and a CDR-2 comprising SEQ ID NO:10; the Vα or Vγ region comprises a CDR-1 comprising SEQ ID NO: 150, and a CDR-2 comprising SEQ ID NO: 151, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO:35, and a CDR-2 comprising SEQ ID NO:36, and the Vβ or Vδ region comprises a CDR-1 comprising SEQ ID NO:9, and a CDR-2 comprising SEQ ID NO:10; the Vα or Vγ region comprises a CDR-1 comprising SEQ ID NO:35, and a CDR-2 comprising SEQ ID NO:36, and the Vβ or Vδ region comprises a CDR-1 comprising SEQ ID NO:9, and a CDR-2 comprising SEQ ID NO:10; the Vα or Vγ region comprises a CDR-1 comprising SEQ ID NO:76, and a CDR-2 comprising SEQ ID NO:77, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO:76, and a CDR-2 comprising SEQ ID NO:77, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO:222, and a CDR-2 comprising SEQ ID NO:223, and the Vβ or Vδ region comprises a CDR-1 comprising SEQ ID NO:9, and a CDR-2 comprising SEQ ID NO:10; the Vα or Vγ region comprises a CDR-1 comprising SEQ ID NO:76, and a CDR-2 comprising SEQ ID NO:77, and the Vβ or Vδ region comprises a CDR-1 comprising SEQ ID NO:9, and a CDR-2 comprising SEQ ID NO: 10; the Vα or Vγ region comprises a CDR-1 comprising SEQ ID NO: 19, and a CDR-2 comprising SEQ ID NO:20, and the Vβ or Vδ region comprises a CDR-1 comprising SEQ ID NO:9, and a CDR-2 comprising SEQ ID NO:10; the Vα or Vγ region comprises a CDR-1 comprising SEQ ID NO:266, and a CDR-2 comprising SEQ ID NO:267, and the Vβ or Vδ region comprises a CDR-1 comprising SEQ ID NO:9, and a CDR-2 comprising SEQ ID NO: 10; or the Vα or Vγ region comprises a CDR-1 comprising SEQ ID NO:19, and a CDR-2 comprising SEQ ID NO:20, and the Vβ or Vδ region comprises a CDR-1 comprising SEQ ID NO:9, and a CDR-2 comprising SEQ ID NO:10.

Also provided herein are TCRs or antigen-binding fragment thereof, comprising: an alpha chain comprising a Vα region and a beta chain comprising a Vβ region; or a gamma chain comprising a Vγ region and a delta chain comprising a Vδ region; wherein: the Vα or Vγ region comprises a CDR-1 comprising SEQ ID NO:1, a CDR-2 comprising SEQ ID NO:2, and a CDR-3 comprising SEQ ID NO:3, and the Vβ or Vδ region comprises a CDR-1 comprising SEQ ID NO:9, a CDR-2 comprising SEQ ID NO: 10, and a CDR-3 comprising SEQ ID NO:11; the Vα or Vγ region comprises a CDR-1 comprising SEQ ID NO: 19, a CDR-2 comprising SEQ ID NO:20, and a CDR-3 comprising SEQ ID NO:21, and the Vβ or Vδ region comprises a CDR-1 comprising SEQ ID NO:9, a CDR-2 comprising SEQ ID NO: 10, and a CDR-3 comprising SEQ ID NO:27; the Vα or Vγ region comprises a CDR-1 comprising SEQ ID NO:35, a CDR-2 comprising SEQ ID NO:36, and a CDR-3 comprising SEQ ID NO:37, and the Vβ or Vδ region comprises a CDR-1 comprising SEQ ID NO:9, a CDR-2 comprising SEQ ID NO: 10, and a CDR-3 comprising SEQ ID NO:43; the Vα or Vγ region comprises a CDR-1 comprising SEQ ID NO:1, a CDR-2 comprising SEQ ID NO:2, and a CDR-3 comprising SEQ ID NO:51, and the Vβ or Vδ region comprises a CDR-1 comprising SEQ ID NO:9, a CDR-2 comprising SEQ ID NO: 10, and a CDR-3 comprising SEQ ID NO:57; the Vα or Vγ region comprises a CDR-1 comprising SEQ ID NO:1, a CDR-2 comprising SEQ ID NO:2, and a CDR-3 comprising SEQ ID NO:65, and the Vβ or Vδ region comprises a CDR-1 comprising SEQ ID NO:9, a CDR-2 comprising SEQ ID NO: 10, and a CDR-3 comprising SEQ ID NO:57; the Vα or Vγ region comprises a CDR-1 comprising SEQ ID NO:76, a CDR-2 comprising SEQ ID NO:77, and a CDR-3 comprising SEQ ID NO:78, and the Vβ or Vδ region comprises a CDR-1 comprising SEQ ID NO:9, a CDR-2 comprising SEQ ID NO: 10, and a CDR-3 comprising SEQ ID NO:84; the Vα or Vγ region comprises a CDR-1 comprising SEQ ID NO: 19, a CDR-2 comprising SEQ ID NO:20, and a CDR-3 comprising SEQ ID NO:92, and the Vβ or Vδ region comprises a CDR-1 comprising SEQ ID NO:9, a CDR-2 comprising SEQ ID NO: 10, and a CDR-3 comprising SEQ ID NO:98; the Vα or Vγ region comprises a CDR-1 comprising SEQ ID NO:76, a CDR-2 comprising SEQ ID NO:77, and a CDR-3 comprising SEQ ID NO: 106, and the Vβ or Vδ region comprises a CDR-1 comprising SEQ ID NO:9, a CDR-2 comprising SEQ ID NO:10, and a CDR-3 comprising SEQ ID NO:112; the Vα or Vγ region comprises a CDR-1 comprising SEQ ID NO:35, a CDR-2 comprising SEQ ID NO:36, and a CDR-3 comprising SEQ ID NO: 120, and the Vβ or Vδ region comprises a CDR-1 comprising SEQ ID NO:9, a CDR-2 comprising SEQ ID NO: 10, and a CDR-3 comprising SEQ ID NO: 126; the Vα or Vγ region comprises a CDR-1 comprising SEQ ID NO: 134, a CDR-2 comprising SEQ ID NO: 135, and a CDR-3 comprising SEQ ID NO:136, and the Vβ or Vδ region comprises a CDR-1 comprising SEQ ID NO:9, a CDR-2 comprising SEQ ID NO: 10, and a CDR-3 comprising SEQ ID NO: 142; the Vα or Vγ region comprises a CDR-1 comprising SEQ ID NO:150, a CDR-2 comprising SEQ ID NO:151, and a CDR-3 comprising SEQ ID NO: 152, and the Vβ or Vδ region comprises a CDR-1 comprising SEQ ID NO:9, a CDR-2 comprising SEQ ID NO: 10, and a CDR-3 comprising SEQ ID NO: 158; the Vα or Vγ region comprises a CDR-1 comprising SEQ ID NO:35, a CDR-2 comprising SEQ ID NO:36, and a CDR-3 comprising SEQ ID NO: 166, and the Vβ or Vδ region comprises a CDR-1 comprising SEQ ID NO:9, a CDR-2 comprising SEQ ID NO: 10, and a CDR-3 comprising SEQ ID NO: 172; the Vα or Vγ region comprises a CDR-1 comprising SEQ ID NO:35, a CDR-2 comprising SEQ ID NO:36, and a CDR-3 comprising SEQ ID NO: 180, and the Vβ or Vδ region comprises a CDR-1 comprising SEQ ID NO:9, a CDR-2 comprising SEQ ID NO:10, and a CDR-3 comprising SEQ ID NO: 186; the Vα or Vγ region comprises a CDR-1 comprising SEQ ID NO:76, a CDR-2 comprising SEQ ID NO:77, and a CDR-3 comprising SEQ ID NO: 194, and the Vβ or Vδ region comprises a CDR-1 comprising SEQ ID NO:9, a CDR-2 comprising SEQ ID NO: 10, and a CDR-3 comprising SEQ ID NO:200; the Vα or Vγ region comprises a CDR-1 comprising SEQ ID NO:76, a CDR-2 comprising SEQ ID NO:77, and a CDR-3 comprising SEQ ID NO:208, and the Vβ or Vδ region comprises a CDR-1 comprising SEQ ID NO:9, a CDR-2 comprising SEQ ID NO: 10, and a CDR-3 comprising SEQ ID NO:214; the Vα or Vγ region comprises a CDR-1 comprising SEQ ID NO:222, a CDR-2 comprising SEQ ID NO:223, and a CDR-3 comprising SEQ ID NO:224, and the Vβ or Vδ region comprises a CDR-1 comprising SEQ ID NO:9, a CDR-2 comprising SEQ ID NO: 10, and a CDR-3 comprising SEQ ID NO:230; the Vα or Vγ region comprises a CDR-1 comprising SEQ ID NO:76, a CDR-2 comprising SEQ ID NO:77, and a CDR-3 comprising SEQ ID NO:238, and the Vβ or Vδ region comprises a CDR-1 comprising SEQ ID NO:9, a CDR-2 comprising SEQ ID NO:10, and a CDR-3 comprising SEQ ID NO:244; the Vα or Vγ region comprises a CDR-1 comprising SEQ ID NO:19, a CDR-2 comprising SEQ ID NO:20, and a CDR-3 comprising SEQ ID NO:252, and the Vβ or Vδ region comprises a CDR-1 comprising SEQ ID NO:9, a CDR-2 comprising SEQ ID NO:10, and a CDR-3 comprising SEQ ID NO:258; the Vα or Vγ region comprises a CDR-1 comprising SEQ ID NO:266, a CDR-2 comprising SEQ ID NO:267, and a CDR-3 comprising SEQ ID NO:268, and the Vβ or Vδ region comprises a CDR-1 comprising SEQ ID NO:9, a CDR-2 comprising SEQ ID NO:10, and a CDR-3 comprising SEQ ID NO: 158; or the Vα or Vγ region comprises a CDR-1 comprising SEQ ID NO:19, a CDR-2 comprising SEQ ID NO:20, and a CDR-3 comprising SEQ ID NO:278, and the Vβ or Vδ region comprises a CDR-1 comprising SEQ ID NO:9, a CDR-2 comprising SEQ ID NO:10, and a CDR-3 comprising SEQ ID NO:284.

Also provided herein are TCRs or antigen-binding fragment thereof, comprising: an alpha chain comprising a Vα region and a beta chain comprising a Vβ region; or a gamma chain comprising a Vγ region and a delta chain comprising a Vδ region; wherein: the Vα or Vγ region comprises a CDR-3 comprising a CDR-3 contained within the Vα or Vγ region sequence of SEQ ID NO:4, and the Vβ or Vδ region comprises a CDR-3 comprising a CDR-3 contained within the Vβ or Vδ region sequence of SEQ ID NO: 12; the Vα or Vγ region comprises a CDR-3 comprising a CDR-3 contained within the Vα or Vγ region sequence of SEQ ID NO:22, and the Vβ or Vδ region comprises a CDR-3 comprising a CDR-3 contained within the Vβ or Vo region sequence of SEQ ID NO:28; the Vα or Vγ region comprises a CDR-3 comprising a CDR-3 contained within the Vα or Vγ region sequence of SEQ ID NO:38, and the Vβ or Vδ region comprises a CDR-3 comprising a CDR-3 contained within the Vβ or Vδ region sequence of SEQ ID NO:44; the Vα or Vγ region comprises a CDR-3 comprising a CDR-3 contained within the Vα or Vγ region sequence of SEQ ID NO:52, and the Vβ or Vδ region comprises a CDR-3 comprising a CDR-3 contained within the Vβ or Vδ region sequence of SEQ ID NO:58; the Vα or Vγ region comprises a CDR-3 comprising a CDR-3 contained within the Vα or Vγ region sequence of SEQ ID NO:66, and the Vβ or Vδ region comprises a CDR-3 comprising a CDR-3 contained within the Vβ or Vδ region sequence of SEQ ID NO:58; the Vα or Vγ region comprises a CDR-3 comprising a CDR-3 contained within the Vα or Vγ region sequence of SEQ ID NO: 79, and the Vβ or Vδ region comprises a CDR-3 comprising a CDR-3 contained within the Vβ or Vδ region sequence of SEQ ID NO:85; the Vα or Vγ region comprises a CDR-3 comprising a CDR-3 contained within the Vα or Vγ region sequence of SEQ ID NO:93, and the Vβ or Vo region comprises a CDR-3 comprising a CDR-3 contained within the Vβ or Vδ region sequence of SEQ ID NO:99; the Vα or Vγ region comprises a CDR-3 comprising a CDR-3 contained within the Vα or Vγ region sequence of SEQ ID NO: 107, and the Vβ or Vδ region comprises a CDR-3 comprising a CDR-3 contained within the Vβ or Vδ region sequence of SEQ ID NO:113; the Vα or Vγ region comprises a CDR-3 comprising a CDR-3 contained within the Vα or Vγ region sequence of SEQ ID NO: 121, and the Vβ or Vδ region comprises a CDR-3 comprising a CDR-3 contained within the Vβ or Vδ region sequence of SEQ ID NO: 127; the Vα or Vγ region comprises a CDR-3 comprising a CDR-3 contained within the Vα or Vγ region sequence of SEQ ID NO: 137, and the Vβ or Vo region comprises a CDR-3 comprising a CDR-3 contained within the Vβ or Vδ region sequence of SEQ ID NO: 143; the Vα or Vγ region comprises a CDR-3 comprising a CDR-3 contained within the Vα or Vγ region sequence of SEQ ID NO:153, and the Vβ or Vo region comprises a CDR-3 comprising a CDR-3 contained within the Vβ or Vδ region sequence of SEQ ID NO: 159; the Vα or Vγ region comprises a CDR-3 comprising a CDR-3 contained within the Vα or Vγ region sequence of SEQ ID NO: 167, and the Vβ or Vδ region comprises a CDR-3 comprising a CDR-3 contained within the Vβ or Vδ region sequence of SEQ ID NO: 173; the Vα or Vγ region comprises a CDR-3 comprising a CDR-3 contained within the Vα or Vγ region sequence of SEQ ID NO: 181, and the Vβ or Vδ region comprises a CDR-3 comprising a CDR-3 contained within the Vβ or Vδ region sequence of SEQ ID NO: 187; the Vα or Vγ region comprises a CDR-3 comprising a CDR-3 contained within the Vα or Vγ region sequence of SEQ ID NO: 195, and the Vβ or Vδ region comprises a CDR-3 comprising a CDR-3 contained within the Vβ or Vδ region sequence of SEQ ID NO:201; the Vα or Vγ region comprises a CDR-3 comprising a CDR-3 contained within the Vα or Vγ region sequence of SEQ ID NO:209, and the Vβ or Vδ region comprises a CDR-3 comprising a CDR-3 contained within the Vβ or Vδ region sequence of SEQ ID NO:215; the Vα or Vγ region comprises a CDR-3 comprising a CDR-3 contained within the Vα or Vγ region sequence of SEQ ID NO:225, and the Vβ or Vδ region comprises a CDR-3 comprising a CDR-3 contained within the Vβ or Vδ region sequence of SEQ ID NO:231; the Vα or Vγ region comprises a CDR-3 comprising a CDR-3 contained within the Vα or Vγ region sequence of SEQ ID NO:239, and the Vβ or Vδ region comprises a CDR-3 comprising a CDR-3 contained within the Vβ or Vδ region sequence of SEQ ID NO:245; the Vα or Vγ region comprises a CDR-3 comprising a CDR-3 contained within the Vα or Vγ region sequence of SEQ ID NO:253, and the Vβ or Vδ region comprises a CDR-3 comprising a CDR-3 contained within the Vβ or Vδ region sequence of SEQ ID NO:259; the Vα or Vγ region comprises a CDR-3 comprising a CDR-3 contained within the Vα or Vγ region sequence of SEQ ID NO:269, and the Vβ or Vδ region comprises a CDR-3 comprising a CDR-3 contained within the Vβ or Vo region sequence of SEQ ID NO: 159; the Vα or Vγ region comprises a CDR-3 comprising a CDR-3 contained within the Vα or Vγ region sequence of SEQ ID NO:279, and the Vβ or Vδ region comprises a CDR-3 comprising a CDR-3 contained within the Vβ or Vδ region sequence of SEQ ID NO:285; the Vα or Vγ region comprises a CDR-3 comprising a CDR-3 contained within the Vα or Vγ region sequence of SEQ ID NO:360, and the Vβ or Vδ region comprises a CDR-3 comprising a CDR-3 contained within the Vβ or Vδ region sequence of SEQ ID NO:364; the Vα or Vγ region comprises a CDR-3 comprising a CDR-3 contained within the Vα or Vγ region sequence of SEQ ID NO:370, and the Vβ or Vδ region comprises a CDR-3 comprising a CDR-3 contained within the Vβ or Vδ region sequence of SEQ ID NO:374; the Vα or Vγ region comprises a CDR-3 comprising a CDR-3 contained within the Vα or Vγ region sequence of SEQ ID NO:380, and the Vβ or Vδ region comprises a CDR-3 comprising a CDR-3 contained within the Vβ or Vδ region sequence of SEQ ID NO:384; the Vα or Vγ region comprises a CDR-3 comprising a CDR-3 contained within the Vα or Vγ region sequence of SEQ ID NO:390, and the Vβ or Vδ region comprises a CDR-3 comprising a CDR-3 contained within the Vβ or Vδ region sequence of SEQ ID NO:394; the Vα or Vγ region comprises a CDR-3 comprising a CDR-3 contained within the Vα or Vγ region sequence of SEQ ID NO:400, and the Vβ or Vδ region comprises a CDR-3 comprising a CDR-3 contained within the Vβ or Vδ region sequence of SEQ ID NO:404; the Vα or Vγ region comprises a CDR-3 comprising a CDR-3 contained within the Vα or Vγ region sequence of SEQ ID NO:410, and the Vβ or Vδ region comprises a CDR-3 comprising a CDR-3 contained within the Vβ or Vδ region sequence of SEQ ID NO:414; the Vα or Vγ region comprises a CDR-3 comprising a CDR-3 contained within the Vα or Vγ region sequence of SEQ ID NO:420, and the Vβ or Vδ region comprises a CDR-3 comprising a CDR-3 contained within the Vβ or Vδ region sequence of SEQ ID NO:424; the Vα or Vγ region comprises a CDR-3 comprising a CDR-3 contained within the Vα or Vγ region sequence of SEQ ID NO:430, and the Vβ or Vo region comprises a CDR-3 comprising a CDR-3 contained within the Vβ or Vδ region sequence of SEQ ID NO:434; the Vα or Vγ region comprises a CDR-3 comprising a CDR-3 contained within the Vα or Vγ region sequence of SEQ ID NO:440, and the Vβ or Vδ region comprises a CDR-3 comprising a CDR-3 contained within the Vβ or Vδ region sequence of SEQ ID NO:444; the Vα or Vγ region comprises a CDR-3 comprising a CDR-3 contained within the Vα or Vγ region sequence of SEQ ID NO:450, and the Vβ or Vδ region comprises a CDR-3 comprising a CDR-3 contained within the Vβ or Vδ region sequence of SEQ ID NO:454; or the Vα or Vγ region comprises a CDR-3 comprising a CDR-3 contained within the Vα or Vγ region sequence of SEQ ID NO:481, and the Vβ or Vδ region comprises a CDR-3 comprising a CDR-3 contained within the Vβ or Vδ region sequence of SEQ ID NO:485.

In some of any of the embodiments provided herein, the Vα or Vγ region comprises a CDR-1 and a CDR-2 comprising a CDR-1 and a CDR-2, respectively, contained within the Vα or Vγ region sequence of SEQ ID NO:4, and the Vβ or Vδ region comprises a CDR-1 and a CDR-2 comprising a CDR-1 and a CDR-2, respectively, contained within the Vβ or Vδ region sequence of SEQ ID NO: 12; the Vα or Vγ region comprises a CDR-1 and a CDR-2 comprising a CDR-1 and a CDR-2, respectively, contained within the Vα or Vγ region sequence of SEQ ID NO:22, and the Vβ or Vδ region comprises a CDR-1 and a CDR-2 comprising a CDR-1 and a CDR-2, respectively, contained within the Vβ or Vδ region sequence of SEQ ID NO:28; the Vα or Vγ region comprises a CDR-1 and a CDR-2 comprising a CDR-1 and a CDR-2, respectively, contained within the Vα or Vγ region sequence of SEQ ID NO:38, and the Vβ or Vδ region comprises a CDR-1 and a CDR-2 comprising a CDR-1 and a CDR-2, respectively, contained within the Vβ or Vδ region sequence of SEQ ID NO:44; the Vα or Vγ region comprises a CDR-1 and a CDR-2 comprising a CDR-1 and a CDR-2, respectively, contained within the Vα or Vγ region sequence of SEQ ID NO:52, and the Vβ or Vδ region comprises a CDR-1 and a CDR-2 comprising a CDR-1 and a CDR-2, respectively, contained within the Vβ or Vδ region sequence of SEQ ID NO:58; the Vα or Vγ region comprises a CDR-1 and a CDR-2 comprising a CDR-1 and a CDR-2, respectively, contained within the Vα or VY region sequence of SEQ ID NO:66, and the Vβ or Vδ region comprises a CDR-1 and a CDR-2 comprising a CDR-1 and a CDR-2, respectively, contained within the Vβ or Vδ region sequence of SEQ ID NO:58; the Vα or Vγ region comprises a CDR-1 and a CDR-2 comprising a CDR-1 and a CDR-2, respectively, contained within the Vα or Vγ region sequence of SEQ ID NO:79, and the Vβ or Vδ region comprises a CDR-1 and a CDR-2 comprising a CDR-1 and a CDR-2, respectively, contained within the Vβ or Vδ region sequence of SEQ ID NO:85; the Vα or Vγ region comprises a CDR-1 and a CDR-2 comprising a CDR-1 and a CDR-2, respectively, contained within the Vα or Vγ region sequence of SEQ ID NO:93, and the Vβ or Vδ region comprises a CDR-1 and a CDR-2 comprising a CDR-1 and a CDR-2, respectively, contained within the Vβ or Vδ region sequence of SEQ ID NO:99; the Vα or Vγ region comprises a CDR-1 and a CDR-2 comprising a CDR-1 and a CDR-2, respectively, contained within the Vα or Vγ region sequence of SEQ ID NO: 107, and the Vβ or Vδ region comprises a CDR-1 and a CDR-2 comprising a CDR-1 and a CDR-2, respectively, contained within the Vβ or Vδ region sequence of SEQ ID NO: 113; the Vα or Vγ region comprises a CDR-1 and a CDR-2 comprising a CDR-1 and a CDR-2, respectively, contained within the Vα or Vγ region sequence of SEQ ID NO:121, and the Vβ or Vδ region comprises a CDR-1 and a CDR-2 comprising a CDR-1 and a CDR-2, respectively, contained within the Vβ or Vδ region sequence of SEQ ID NO: 127; the Vα or Vγ region comprises a CDR-1 and a CDR-2 comprising a CDR-1 and a CDR-2, respectively, contained within the Vα or Vγ region sequence of SEQ ID NO: 137, and the Vβ or Vδ region comprises a CDR-1 and a CDR-2 comprising a CDR-1 and a CDR-2, respectively, contained within the Vβ or Vδ region sequence of SEQ ID NO: 143; the Vα or Vγ region comprises a CDR-1 and a CDR-2 comprising a CDR-1 and a CDR-2, respectively, contained within the Vα or Vγ region sequence of SEQ ID NO: 153, and the Vβ or Vδ region comprises a CDR-1 and a CDR-2 comprising a CDR-1 and a CDR-2, respectively, contained within the Vβ or Vδ region sequence of SEQ ID NO: 159; the Vα or Vγ region comprises a CDR-1 and a CDR-2 comprising a CDR-1 and a CDR-2, respectively, contained within the Vα or Vγ region sequence of SEQ ID NO: 167, and the Vβ or Vδ region comprises a CDR-1 and a CDR-2 comprising a CDR-1 and a CDR-2, respectively, contained within the Vβ or Vδ region sequence of SEQ ID NO:173; the Vα or Vγ region comprises a CDR-1 and a CDR-2 comprising a CDR-1 and a CDR-2, respectively, contained within the Vα or Vγ region sequence of SEQ ID NO: 181, and the Vβ or Vδ region comprises a CDR-1 and a CDR-2 comprising a CDR-1 and a CDR-2, respectively, contained within the Vβ or Vδ region sequence of SEQ ID NO:187; the Vα or Vγ region comprises a CDR-1 and a CDR-2 comprising a CDR-1 and a CDR-2, respectively, contained within the Vα or Vγ region sequence of SEQ ID NO: 195, and the Vβ or Vδ region comprises a CDR-1 and a CDR-2 comprising a CDR-1 and a CDR-2, respectively, contained within the Vβ or Vδ region sequence of SEQ ID NO:201; the Vα or Vγ region comprises a CDR-1 and a CDR-2 comprising a CDR-1 and a CDR-2, respectively, contained within the Vα or Vγ region sequence of SEQ ID NO:209, and the Vβ or Vo region comprises a CDR-1 and a CDR-2 comprising a CDR-1 and a CDR-2, respectively, contained within the Vβ or Vδ region sequence of SEQ ID NO:215; the Vα or Vγ region comprises a CDR-1 and a CDR-2 comprising a CDR-1 and a CDR-2, respectively, contained within the Vα or Vγ region sequence of SEQ ID NO:225, and the Vβ or Vδ region comprises a CDR-1 and a CDR-2 comprising a CDR-1 and a CDR-2, respectively, contained within the Vβ or Vδ region sequence of SEQ ID NO:231; the Vα or Vγ region comprises a CDR-1 and a CDR-2 comprising a CDR-1 and a CDR-2, respectively, contained within the Vα or Vγ region sequence of SEQ ID NO:239, and the Vβ or Vδ region comprises a CDR-1 and a CDR-2 comprising a CDR-1 and a CDR-2, respectively, contained within the Vβ or Vδ region sequence of SEQ ID NO:245; the Vα or Vγ region comprises a CDR-1 and a CDR-2 comprising a CDR-1 and a CDR-2, respectively, contained within the Vα or Vγ region sequence of SEQ ID NO:253, and the Vβ or Vδ region comprises a CDR-1 and a CDR-2 comprising a CDR-1 and a CDR-2, respectively, contained within the Vβ or Vδ region sequence of SEQ ID NO:259; the Vα or Vγ region comprises a CDR-1 and a CDR-2 comprising a CDR-1 and a CDR-2, respectively, contained within the Vα or Vγ region sequence of SEQ ID NO:269, and the Vβ or Vδ region comprises a CDR-1 and a CDR-2 comprising a CDR-1 and a CDR-2, respectively, contained within the Vβ or Vδ region sequence of SEQ ID NO: 159; the Vα or Vγ region comprises a CDR-1 and a CDR-2 comprising a CDR-1 and a CDR-2, respectively, contained within the Vα or Vγ region sequence of SEQ ID NO:279, and the Vβ or Vδ region comprises a CDR-1 and a CDR-2 comprising a CDR-1 and a CDR-2, respectively, contained within the Vβ or Vδ region sequence of SEQ ID NO:285; the Vα or Vγ region comprises a CDR-1 and a CDR-2 comprising a CDR-1 and a CDR-2, respectively, contained within the Vα or Vγ region sequence of SEQ ID NO:360, and the Vβ or Vo region comprises a CDR-1 and a CDR-2 comprising a CDR-1 and a CDR-2, respectively, contained within the Vβ or Vδ region sequence of SEQ ID NO:364; the Vα or Vγ region comprises a CDR-1 and a CDR-2 comprising a CDR-1 and a CDR-2, respectively, contained within the Vα or Vγ region sequence of SEQ ID NO:370, and the Vβ or Vδ region comprises a CDR-1 and a CDR-2 comprising a CDR-1 and a CDR-2, respectively, contained within the Vβ or Vδ region sequence of SEQ ID NO:374; the Vα or Vγ region comprises a CDR-1 and a CDR-2 comprising a CDR-1 and a CDR-2, respectively, contained within the Vα or VY region sequence of SEQ ID NO:380, and the Vβ or Vδ region comprises a CDR-1 and a CDR-2 comprising a CDR-1 and a CDR-2, respectively, contained within the Vβ or Vδ region sequence of SEQ ID NO:384; the Vα or Vγ region comprises a CDR-1 and a CDR-2 comprising a CDR-1 and a CDR-2, respectively, contained within the Vα or Vγ region sequence of SEQ ID NO:390, and the Vβ or Vδ region comprises a CDR-1 and a CDR-2 comprising a CDR-1 and a CDR-2, respectively, contained within the Vβ or Vδ region sequence of SEQ ID NO:394; the Vα or Vγ region comprises a CDR-1 and a CDR-2 comprising a CDR-1 and a CDR-2, respectively, contained within the Vα or VY region sequence of SEQ ID NO:400, and the Vβ or Vδ region comprises a CDR-1 and a CDR-2 comprising a CDR-1 and a CDR-2, respectively, contained within the Vβ or Vδ region sequence of SEQ ID NO:404; the Vα or Vγ region comprises a CDR-1 and a CDR-2 comprising a CDR-1 and a CDR-2, respectively, contained within the Vα or Vγ region sequence of SEQ ID NO:410, and the Vβ or Vδ region comprises a CDR-1 and a CDR-2 comprising a CDR-1 and a CDR-2, respectively, contained within the Vβ or Vδ region sequence of SEQ ID NO:414; the Vα or Vγ region comprises a CDR-1 and a CDR-2 comprising a CDR-1 and a CDR-2, respectively, contained within the Vα or Vγ region sequence of SEQ ID NO:420, and the Vβ or Vδ region comprises a CDR-1 and a CDR-2 comprising a CDR-1 and a CDR-2, respectively, contained within the Vβ or Vδ region sequence of SEQ ID NO:424; the Vα or Vγ region comprises a CDR-1 and a CDR-2 comprising a CDR-1 and a CDR-2, respectively, contained within the Vα or Vγ region sequence of SEQ ID NO:430, and the Vβ or Vδ region comprises a CDR-1 and a CDR-2 comprising a CDR-1 and a CDR-2, respectively, contained within the Vβ or Vδ region sequence of SEQ ID NO:434; the Vα or Vγ region comprises a CDR-1 and a CDR-2 comprising a CDR-1 and a CDR-2, respectively, contained within the Vα or Vγ region sequence of SEQ ID NO:440, and the Vβ or Vδ region comprises a CDR-1 and a CDR-2 comprising a CDR-1 and a CDR-2, respectively, contained within the Vβ or Vδ region sequence of SEQ ID NO:444; the Vα or Vγ region comprises a CDR-1 and a CDR-2 comprising a CDR-1 and a CDR-2, respectively, contained within the Vα or Vγ region sequence of SEQ ID NO:450, and the Vβ or Vδ region comprises a CDR-1 and a CDR-2 comprising a CDR-1 and a CDR-2, respectively, contained within the Vβ or Vδ region sequence of SEQ ID NO:454; or the Vα or Vγ region comprises a CDR-1 and a CDR-2 comprising a CDR-1 and a CDR-2, respectively, contained within the Vα or Vγ region sequence of SEQ ID NO:481, and the Vβ or Vδ region comprises a CDR-1 and a CDR-2 comprising a CDR-1 and a CDR-2, respectively, contained within the Vβ or Vδ region sequence of SEQ ID NO:485.

Also provided are TCRs or antigen-binding fragment thereof, comprising: an alpha chain comprising a Vα region and a beta chain comprising a Vβ region; or a gamma chain comprising a Vγ region and a delta chain comprising a Vδ region; wherein: the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:4, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO: 12; the Vα or Vγ region comprises a CDR-1, a CDR-2, and a CDR-3 comprising a CDR-1, a CDR-2, and a CDR-3, respectively, contained within the Vα or VY region sequence of SEQ ID NO:22, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:28; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:38, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:44; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:52, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:58; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:66, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:58; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO: 79, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:85; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:93, and the Vβ or Vo region comprises a CDR-1, a CDR-2, and a CDR-3 comprising a CDR-1, a CDR-2, and a CDR-3, respectively, contained within the Vβ or Vδ region sequence of SEQ ID NO:99; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO: 107, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:113; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO: 121, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO: 127; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO: 137, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:143; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO: 153, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO: 159; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO: 167, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO: 173; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO: 181, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO: 187; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO: 195, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:201; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:209, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:215; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:225, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:231; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:239, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:245; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:253, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:259; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:269, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO: 159; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:279, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:285; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:360, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:364; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:370, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:374; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:380, and the Vβ or Vo region comprises a CDR-1, a CDR-2, and a CDR-3 comprising a CDR-1, a CDR-2, and a CDR-3, respectively, contained within the Vβ or Vδ region sequence of SEQ ID NO:384; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:390, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:394; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:400, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:404; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:410, and the Vβ or Vo region comprises a CDR-1, a CDR-2, and a CDR-3 comprising a CDR-1, a CDR-2, and a CDR-3, respectively, contained within the Vβ or Vδ region sequence of SEQ ID NO:414; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:420, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:424; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:430, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:434; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:440, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:444; the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:450, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:454; or the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:481, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:485.

Also provided are TCRs or antigen-binding fragment thereof, comprising: an alpha chain comprising a Vα region and a beta chain comprising a Vβ region; or a gamma chain comprising a Vγ region and a delta chain comprising a Vδ region; wherein: the Vα or Vγ region comprises SEQ ID NO:4 or a sequence that has at least 90% sequence identity thereto, and the Vβ or Vδ region comprises SEQ ID NO:12 or a sequence that has at least 90% sequence identity thereto; the Vα or Vγ region comprises SEQ ID NO:22 or a sequence that has at least 90% sequence identity thereto, and the Vβ or Vδ region comprises SEQ ID NO:28 or a sequence that has at least 90% sequence identity thereto; the Vα or Vγ region comprises SEQ ID NO:38 or a sequence that has at least 90% sequence identity thereto, and the Vβ or Vδ region comprises SEQ ID NO:44 or a sequence that has at least 90% sequence identity thereto; the Vα or Vγ region comprises SEQ ID NO:52 or a sequence that has at least 90% sequence identity thereto, and the Vβ or Vδ region comprises SEQ ID NO:58 or a sequence that has at least 90% sequence identity thereto; the Vα or Vγ region comprises SEQ ID NO:66 or a sequence that has at least 90% sequence identity thereto, and the Vβ or Vδ region comprises SEQ ID NO:58 or a sequence that has at least 90% sequence identity thereto; the Vα or Vγ region comprises SEQ ID NO: 79 or a sequence that has at least 90% sequence identity thereto, and the Vβ or Vδ region comprises SEQ ID NO:85 or a sequence that has at least 90% sequence identity thereto; the Vα or Vγ region comprises SEQ ID NO:93 or a sequence that has at least 90% sequence identity thereto, and the Vβ or Vδ region comprises SEQ ID NO:99 or a sequence that has at least 90% sequence identity thereto; the Vα or Vγ region comprises SEQ ID NO: 107 or a sequence that has at least 90% sequence identity thereto, and the Vβ or Vδ region comprises SEQ ID NO:113 or a sequence that has at least 90% sequence identity thereto; the Vα or Vγ region comprises SEQ ID NO:121 or a sequence that has at least 90% sequence identity thereto, and the Vβ or Vδ region comprises SEQ ID NO: 127 or a sequence that has at least 90% sequence identity thereto; the Vα or Vγ region comprises SEQ ID NO:137 or a sequence that has at least 90% sequence identity thereto, and the Vβ or Vδ region comprises SEQ ID NO: 143 or a sequence that has at least 90% sequence identity thereto; the Vα or Vγ region comprises SEQ ID NO: 153 or a sequence that has at least 90% sequence identity thereto, and the Vβ or Vδ region comprises SEQ ID NO:159 or a sequence that has at least 90% sequence identity thereto; the Vα or Vγ region comprises SEQ ID NO: 167 or a sequence that has at least 90% sequence identity thereto, and the Vβ or Vδ region comprises SEQ ID NO: 173 or a sequence that has at least 90% sequence identity thereto; the Vα or Vγ region comprises SEQ ID NO: 181 or a sequence that has at least 90% sequence identity thereto, and the Vβ or Vδ region comprises SEQ ID NO:187 or a sequence that has at least 90% sequence identity thereto; the Vα or Vγ region comprises SEQ ID NO:195 or a sequence that has at least 90% sequence identity thereto, and the Vβ or Vδ region comprises SEQ ID NO:201 or a sequence that has at least 90% sequence identity thereto; the Vα or Vγ region comprises SEQ ID NO:209 or a sequence that has at least 90% sequence identity thereto, and the Vβ or Vδ region comprises SEQ ID NO:215 or a sequence that has at least 90% sequence identity thereto; the Vα or Vγ region comprises SEQ ID NO:225 or a sequence that has at least 90% sequence identity thereto, and the Vβ or Vδ region comprises SEQ ID NO:231 or a sequence that has at least 90% sequence identity thereto; the Vα or Vγ region comprises SEQ ID NO:239 or a sequence that has at least 90% sequence identity thereto, and the Vβ or Vδ region comprises SEQ ID NO:245 or a sequence that has at least 90% sequence identity thereto; the Vα or Vγ region comprises SEQ ID NO:253 or a sequence that has at least 90% sequence identity thereto, and the Vβ or Vδ region comprises SEQ ID NO:259 or a sequence that has at least 90% sequence identity thereto; the Vα or Vγ region comprises SEQ ID NO:269 or a sequence that has at least 90% sequence identity thereto, and the Vβ or Vδ region comprises SEQ ID NO: 159 or a sequence that has at least 90% sequence identity thereto; the Vα or Vγ region comprises SEQ ID NO:279 or a sequence that has at least 90% sequence identity thereto, and the Vβ or Vδ region comprises SEQ ID NO:285 or a sequence that has at least 90% sequence identity thereto; the Vα or Vγ region comprises SEQ ID NO:360 or a sequence that has at least 90% sequence identity thereto, and the Vβ or Vδ region comprises SEQ ID NO:364 or a sequence that has at least 90% sequence identity thereto; the Vα or Vγ region comprises SEQ ID NO:370 or a sequence that has at least 90% sequence identity thereto, and the Vβ or Vδ region comprises SEQ ID NO:374 or a sequence that has at least 90% sequence identity thereto; the Vα or Vγ region comprises SEQ ID NO:380 or a sequence that has at least 90% sequence identity thereto, and the Vβ or Vδ region comprises SEQ ID NO:384 or a sequence that has at least 90% sequence identity thereto; the Vα or Vγ region comprises SEQ ID NO:390 or a sequence that has at least 90% sequence identity thereto, and the Vβ or Vδ region comprises SEQ ID NO:394 or a sequence that has at least 90% sequence identity thereto; the Vα or Vγ region comprises SEQ ID NO:400 or a sequence that has at least 90% sequence identity thereto, and the Vβ or Vδ region comprises SEQ ID NO:404 or a sequence that has at least 90% sequence identity thereto; the Vα or Vγ region comprises SEQ ID NO:410 or a sequence that has at least 90% sequence identity thereto, and the Vβ or Vδ region comprises SEQ ID NO:414 or a sequence that has at least 90% sequence identity thereto; the Vα or Vγ region comprises SEQ ID NO:420 or a sequence that has at least 90% sequence identity thereto, and the Vβ or Vδ region comprises SEQ ID NO:424 or a sequence that has at least 90% sequence identity thereto; the Vα or Vγ region comprises SEQ ID NO:430 or a sequence that has at least 90% sequence identity thereto, and the Vβ or Vδ region comprises SEQ ID NO:434 or a sequence that has at least 90% sequence identity thereto; the Vα or Vγ region comprises SEQ ID NO:440 or a sequence that has at least 90% sequence identity thereto, and the Vβ or Vδ region comprises SEQ ID NO:444 or a sequence that has at least 90% sequence identity thereto; the Vα or Vγ region comprises SEQ ID NO: 450 or a sequence that has at least 90% sequence identity thereto, and the Vβ or Vδ region comprises SEQ ID NO:454 or a sequence that has at least 90% sequence identity thereto; or the Vα or Vγ region comprises SEQ ID NO: 481 or a sequence that has at least 90% sequence identity thereto, and the Vβ or Vδ region comprises SEQ ID NO:485 or a sequence that has at least 90% sequence identity thereto.

In some of any of the embodiments provided herein, the Vα or Vγ region comprises SEQ ID NO:4, and the Vβ or Vδ region comprises SEQ ID NO: 12; the Vα or Vγ region comprises SEQ ID NO:22, and the Vβ or Vδ region comprises SEQ ID NO:28; the Vα or Vγ region comprises SEQ ID NO:38, and the Vβ or Vδ region comprises SEQ ID NO:44; the Vα or Vγ region comprises SEQ ID NO:52, and the Vβ or Vδ region comprises SEQ ID NO:58; the Vα or Vγ region comprises SEQ ID NO:66, and the Vβ or Vδ region comprises SEQ ID NO:58; the Vα or Vγ region comprises SEQ ID NO:79, and the Vβ or Vδ region comprises SEQ ID NO:85; the Vα or Vγ region comprises SEQ ID NO:93, and the Vβ or Vδ region comprises SEQ ID NO:99; the Vα or Vγ region comprises SEQ ID NO: 107, and the Vβ or Vδ region comprises SEQ ID NO:113; the Vα or Vγ region comprises SEQ ID NO: 121, and the Vβ or Vδ region comprises SEQ ID NO: 127; the Vα or Vγ region comprises SEQ ID NO: 137, and the Vβ or Vδ region comprises SEQ ID NO: 143; the Vα or Vγ region comprises SEQ ID NO: 153, and the Vβ or Vδ region comprises SEQ ID NO: 159; the Vα or Vγ region comprises SEQ ID NO:167, and the Vβ or Vδ region comprises SEQ ID NO: 173; the Vα or Vγ region comprises SEQ ID NO: 181, and the Vβ or Vδ region comprises SEQ ID NO: 187; the Vα or Vγ region comprises SEQ ID NO: 195, and the Vβ or Vδ region comprises SEQ ID NO:201; the Vα or Vγ region comprises SEQ ID NO:209, and the Vβ or Vδ region comprises SEQ ID NO:215; the Vα or Vγ region comprises SEQ ID NO:225, and the Vβ or Vδ region comprises SEQ ID NO:231; the Vα or Vγ region comprises SEQ ID NO:239, and the Vβ or Vδ region comprises SEQ ID NO:245; the Vα or Vγ region comprises SEQ ID NO:253, and the Vβ or Vδ region comprises SEQ ID NO:259; the Vα or Vγ region comprises SEQ ID NO:269, and the Vβ or Vo region comprises SEQ ID NO: 159; the Vα or Vγ region comprises SEQ ID NO:279, and the Vβ or Vδ region comprises SEQ ID NO:285; the Vα or Vγ region comprises SEQ ID NO:360, and the Vβ or Vδ region comprises SEQ ID NO:364; the Vα or Vγ region comprises SEQ ID NO:370, and the Vβ or Vδ region comprises SEQ ID NO:374; the Vα or Vγ region comprises SEQ ID NO:380, and the Vβ or Vδ region comprises SEQ ID NO:384; the Vα or Vγ region comprises SEQ ID NO:390, and the Vβ or Vδ region comprises SEQ ID NO:394; the Vα or Vγ region comprises SEQ ID NO:400, and the Vβ or Vδ region comprises SEQ ID NO:404; the Vα or Vγ region comprises SEQ ID NO:410, and the Vβ or Vδ region comprises SEQ ID NO:414; the Vα or Vγ region comprises SEQ ID NO:420, and the Vβ or Vδ region comprises SEQ ID NO:424; the Vα or Vγ region comprises SEQ ID NO:430, and the Vβ or Vδ region comprises SEQ ID NO:434; the Vα or Vγ region comprises SEQ ID NO:440, and the Vβ or Vδ region comprises SEQ ID NO:444; the Vα or Vγ region comprises SEQ ID NO:450, and the Vβ or Vδ region comprises SEQ ID NO:454; or the Vα or Vγ region comprises SEQ ID NO:481, and the Vβ or Vδ region comprises SEQ ID NO:485.

In some of any of the embodiments provided herein, the alpha chain further comprises an alpha constant (Cα) region and the beta chain further comprises a beta constant (Cβ) region; or the gamma chain further comprises an gamma constant (Cδ) region and the delta chain further comprises a delta constant (Cδ) region. In some of any of the embodiments provided herein, the Cα comprises SEQ ID NO: 294 or 296, and the Cβ comprises SEQ ID NO: 297 or 299.

In some of any of the embodiments provided herein, the alpha or gamma chain comprises SEQ ID NO:6, and the beta or delta chain comprises SEQ ID NO:14; the alpha or gamma chain comprises SEQ ID NO:24, and the beta or delta chain comprises SEQ ID NO:30; the alpha or gamma chain comprises SEQ ID NO:40, and the beta or delta chain comprises SEQ ID NO:46; the alpha or gamma chain comprises SEQ ID NO:54, and the beta or delta chain comprises SEQ ID NO:60; the alpha or gamma chain comprises SEQ ID NO:68, and the beta or delta chain comprises SEQ ID NO:71; the alpha or gamma chain comprises SEQ ID NO:81, and the beta or delta chain comprises SEQ ID NO:87; the alpha or gamma chain comprises SEQ ID NO:95, and the beta or delta chain comprises SEQ ID NO: 101; the alpha or gamma chain comprises SEQ ID NO: 109, and the beta or delta chain comprises SEQ ID NO:115; the alpha or gamma chain comprises SEQ ID NO:123, and the beta or delta chain comprises SEQ ID NO:129; the alpha or gamma chain comprises SEQ ID NO:139, and the beta or delta chain comprises SEQ ID NO: 145; the alpha or gamma chain comprises SEQ ID NO:155, and the beta or delta chain comprises SEQ ID NO:161; the alpha or gamma chain comprises SEQ ID NO: 169, and the beta or delta chain comprises SEQ ID NO:175; the alpha or gamma chain comprises SEQ ID NO: 183, and the beta or delta chain comprises SEQ ID NO: 189; the alpha or gamma chain comprises SEQ ID NO:197, and the beta or delta chain comprises SEQ ID NO:203; the alpha or gamma chain comprises SEQ ID NO:211, and the beta or delta chain comprises SEQ ID NO:217; the alpha or gamma chain comprises SEQ ID NO:227, and the beta or delta chain comprises SEQ ID NO:233; the alpha or gamma chain comprises SEQ ID NO:241, and the beta or delta chain comprises SEQ ID NO:247; the alpha or gamma chain comprises SEQ ID NO:255, and the beta or delta chain comprises SEQ ID NO:261; the alpha or gamma chain comprises SEQ ID NO:271, and the beta or delta chain comprises SEQ ID NO: 161; the alpha or gamma chain comprises SEQ ID NO:281, and the beta or delta chain comprises SEQ ID NO:287; the alpha or gamma chain comprises SEQ ID NO:362, and the beta or delta chain comprises SEQ ID NO:366; the alpha or gamma chain comprises SEQ ID NO:372, and the beta or delta chain comprises SEQ ID NO:376; the alpha or gamma chain comprises SEQ ID NO:382, and the beta or delta chain comprises SEQ ID NO:386; the alpha or gamma chain comprises SEQ ID NO:392, and the beta or delta chain comprises SEQ ID NO:396; the alpha or gamma chain comprises SEQ ID NO:402, and the beta or delta chain comprises SEQ ID NO:406; the alpha or gamma chain comprises SEQ ID NO:412, and the beta or delta chain comprises SEQ ID NO:416; the alpha or gamma chain comprises SEQ ID NO:422, and the beta or delta chain comprises SEQ ID NO:426; the alpha or gamma chain comprises SEQ ID NO:432, and the beta or delta chain comprises SEQ ID NO:436; the alpha or gamma chain comprises SEQ ID NO:442, and the beta or delta chain comprises SEQ ID NO:446; the alpha or gamma chain comprises SEQ ID NO:452, and the beta or delta chain comprises SEQ ID NO:456; or the alpha or gamma chain comprises SEQ ID NO:483, and the beta or delta chain comprises SEQ ID NO:487.

In some of any of the embodiments provided herein, the TCR or antigen-binding fragment thereof recognizes a peptide epitope of a minor histocompatibility antigen HA-1 in the context of an MHC molecule. In some of any of the embodiments provided herein, the MHC molecule is a human leukocyte antigens (HLA)-A molecule. In some of any of the embodiments provided herein, the HLA-A molecule is of serotype HLA-A*02:01. In some of any of the embodiments provided herein, the HLA-A molecule is of serotype HLA-A*02:06. In some of any of the embodiments provided herein, the peptide epitope of HA-1 is set forth in SEQ ID NO:354.

Also provided are polynucleotides encoding any of the TCRs or antigen-binding fragment thereof provided herein, or an alpha chain, a beta chain, a gamma chain, or a delta chain thereof.

In some of any of the embodiments provided herein, the polynucleotide comprises a nucleotide sequence encoding the Vα region and a nucleotide sequence encoding the Vβ region; or a nucleotide sequence encoding the Vγ region and a nucleotide sequence encoding the Vδ region; wherein: the nucleotide sequence encoding the Vα or Vγ region comprises SEQ ID NO:7 or a sequence that has at least 90% sequence identity thereto, and the nucleotide sequence encoding the Vβ or Vδ region comprises SEQ ID NO:15 or a sequence that has at least 90% sequence identity thereto; the nucleotide sequence encoding the Vα or Vγ region comprises SEQ ID NO:25 or a sequence that has at least 90% sequence identity thereto, and the nucleotide sequence encoding the Vβ or Vδ region comprises SEQ ID NO:31 or a sequence that has at least 90% sequence identity thereto; the nucleotide sequence encoding the Vα or Vγ region comprises SEQ ID NO:41 or a sequence that has at least 90% sequence identity thereto, and the nucleotide sequence encoding the Vβ or Vδ region comprises SEQ ID NO:47 or a sequence that has at least 90% sequence identity thereto; the nucleotide sequence encoding the Vα or Vγ region comprises SEQ ID NO:55 or a sequence that has at least 90% sequence identity thereto, and the nucleotide sequence encoding the Vβ or Vδ region comprises SEQ ID NO:61 or a sequence that has at least 90% sequence identity thereto; the nucleotide sequence encoding the Vα or Vγ region comprises SEQ ID NO:69 or a sequence that has at least 90% sequence identity thereto, and the nucleotide sequence encoding the Vβ or Vδ region comprises SEQ ID NO:72 or a sequence that has at least 90% sequence identity thereto; the nucleotide sequence encoding the Vα or Vγ region comprises SEQ ID NO:82 or a sequence that has at least 90% sequence identity thereto, and the nucleotide sequence encoding the Vβ or Vδ region comprises SEQ ID NO:88 or a sequence that has at least 90% sequence identity thereto; the nucleotide sequence encoding the Vα or Vγ region comprises SEQ ID NO:96 or a sequence that has at least 90% sequence identity thereto, and the nucleotide sequence encoding the Vβ or Vδ region comprises SEQ ID NO: 102 or a sequence that has at least 90% sequence identity thereto; the nucleotide sequence encoding the Vα or Vγ region comprises SEQ ID NO: 110 or a sequence that has at least 90% sequence identity thereto, and the nucleotide sequence encoding the Vβ or Vδ region comprises SEQ ID NO:116 or a sequence that has at least 90% sequence identity thereto; the nucleotide sequence encoding the Vα or Vγ region comprises SEQ ID NO: 124 or a sequence that has at least 90% sequence identity thereto, and the nucleotide sequence encoding the Vβ or Vδ region comprises SEQ ID NO:130 or a sequence that has at least 90% sequence identity thereto; the nucleotide sequence encoding the Vα or Vγ region comprises SEQ ID NO:140 or a sequence that has at least 90% sequence identity thereto, and the nucleotide sequence encoding the Vβ or Vδ region comprises SEQ ID NO: 146 or a sequence that has at least 90% sequence identity thereto; the nucleotide sequence encoding the Vα or Vγ region comprises SEQ ID NO:156 or a sequence that has at least 90% sequence identity thereto, and the nucleotide sequence encoding the Vβ or Vδ region comprises SEQ ID NO:162 or a sequence that has at least 90% sequence identity thereto; the nucleotide sequence encoding the Vα or Vγ region comprises SEQ ID NO:170 or a sequence that has at least 90% sequence identity thereto, and the nucleotide sequence encoding the Vβ or Vδ region comprises SEQ ID NO:176 or a sequence that has at least 90% sequence identity thereto; the nucleotide sequence encoding the Vα or Vγ region comprises SEQ ID NO:184 or a sequence that has at least 90% sequence identity thereto, and the nucleotide sequence encoding the Vβ or Vδ region comprises SEQ ID NO:190 or a sequence that has at least 90% sequence identity thereto; the nucleotide sequence encoding the Vα or Vγ region comprises SEQ ID NO: 198 or a sequence that has at least 90% sequence identity thereto, and the nucleotide sequence encoding the Vβ or Vδ region comprises SEQ ID NO:204 or a sequence that has at least 90% sequence identity thereto; the nucleotide sequence encoding the Vα or Vγ region comprises SEQ ID NO:212 or a sequence that has at least 90% sequence identity thereto, and the nucleotide sequence encoding the Vβ or Vδ region comprises SEQ ID NO:218 or a sequence that has at least 90% sequence identity thereto; the nucleotide sequence encoding the Vα or Vγ region comprises SEQ ID NO:228 or a sequence that has at least 90% sequence identity thereto, and the nucleotide sequence encoding the Vβ or Vδ region comprises SEQ ID NO:234 or a sequence that has at least 90% sequence identity thereto; the nucleotide sequence encoding the Vα or Vγ region comprises SEQ ID NO:242 or a sequence that has at least 90% sequence identity thereto, and the nucleotide sequence encoding the Vβ or Vδ region comprises SEQ ID NO:248 or a sequence that has at least 90% sequence identity thereto; the nucleotide sequence encoding the Vα or Vγ region comprises SEQ ID NO:256 or a sequence that has at least 90% sequence identity thereto, and the nucleotide sequence encoding the Vβ or Vδ region comprises SEQ ID NO:262 or a sequence that has at least 90% sequence identity thereto; the nucleotide sequence encoding the Vα or Vγ region comprises SEQ ID NO:272 or a sequence that has at least 90% sequence identity thereto, and the nucleotide sequence encoding the Vβ or Vδ region comprises SEQ ID NO:274 or a sequence that has at least 90% sequence identity thereto; or the nucleotide sequence encoding the Vα or Vγ region comprises SEQ ID NO:282 or a sequence that has at least 90% sequence identity thereto, and the nucleotide sequence encoding the Vβ or Vδ region comprises SEQ ID NO:288 or a sequence that has at least 90% sequence identity thereto.

In some of any of the embodiments provided herein, the nucleotide sequence encoding the alpha or gamma chain comprises SEQ ID NO:8, and the nucleotide sequence encoding the beta or delta chain comprises SEQ ID NO:16; the nucleotide sequence encoding the alpha or gamma chain comprises SEQ ID NO:26, and the nucleotide sequence encoding the beta or delta chain comprises SEQ ID NO:32; the nucleotide sequence encoding the alpha or gamma chain comprises SEQ ID NO:42, and the nucleotide sequence encoding the beta or delta chain comprises SEQ ID NO:48; the nucleotide sequence encoding the alpha or gamma chain comprises SEQ ID NO:56, and the nucleotide sequence encoding the beta or delta chain comprises SEQ ID NO:62; the nucleotide sequence encoding the alpha or gamma chain comprises SEQ ID NO:70, and the nucleotide sequence encoding the beta or delta chain comprises SEQ ID NO:73; the nucleotide sequence encoding the alpha or gamma chain comprises SEQ ID NO:83, and the nucleotide sequence encoding the beta or delta chain comprises SEQ ID NO:89; the nucleotide sequence encoding the alpha or gamma chain comprises SEQ ID NO:97, and the nucleotide sequence encoding the beta or delta chain comprises SEQ ID NO:103; the nucleotide sequence encoding the alpha or gamma chain comprises SEQ ID NO:111, and the nucleotide sequence encoding the beta or delta chain comprises SEQ ID NO:117; the nucleotide sequence encoding the alpha or gamma chain comprises SEQ ID NO: 125, and the nucleotide sequence encoding the beta or delta chain comprises SEQ ID NO:131; the nucleotide sequence encoding the alpha or gamma chain comprises SEQ ID NO:141, and the nucleotide sequence encoding the beta or delta chain comprises SEQ ID NO:147; the nucleotide sequence encoding the alpha or gamma chain comprises SEQ ID NO:157, and the nucleotide sequence encoding the beta or delta chain comprises SEQ ID NO:163; the nucleotide sequence encoding the alpha or gamma chain comprises SEQ ID NO: 171, and the nucleotide sequence encoding the beta or delta chain comprises SEQ ID NO:177; the nucleotide sequence encoding the alpha or gamma chain comprises SEQ ID NO: 185, and the nucleotide sequence encoding the beta or delta chain comprises SEQ ID NO:191; the nucleotide sequence encoding the alpha or gamma chain comprises SEQ ID NO:199, and the nucleotide sequence encoding the beta or delta chain comprises SEQ ID NO:205; the nucleotide sequence encoding the alpha or gamma chain comprises SEQ ID NO:213, and the nucleotide sequence encoding the beta or delta chain comprises SEQ ID NO:219; the nucleotide sequence encoding the alpha or gamma chain comprises SEQ ID NO:229, and the nucleotide sequence encoding the beta or delta chain comprises SEQ ID NO:235; the nucleotide sequence encoding the alpha or gamma chain comprises SEQ ID NO:243, and the nucleotide sequence encoding the beta or delta chain comprises SEQ ID NO:249; the nucleotide sequence encoding the alpha or gamma chain comprises SEQ ID NO:257, and the nucleotide sequence encoding the beta or delta chain comprises SEQ ID NO:263; the nucleotide sequence encoding the alpha or gamma chain comprises SEQ ID NO:273, and the nucleotide sequence encoding the beta or delta chain comprises SEQ ID NO:275; or the nucleotide sequence encoding the alpha or gamma chain comprises SEQ ID NO:283, and the nucleotide sequence encoding the beta or delta chain comprises SEQ ID NO:289. In some embodiments, the nucleotide sequences encoding the alpha or gamma chain and the nucleotide sequence encoding the beta or delta chain comprise sequences present in SEQ ID NO: 368, 378, 388, 398, 408, 418, 428, 438, 448, 458, or 489. Nucleotides of SEQ ID NO: 368, 378, 388, 398, 408, 418, 428, 438, 448, 458, and 489 encoding the alpha chains, P2A sequences, and beta chains are indicated in the sequence Table 4.

In some of any of the embodiments provided herein, the nucleotide sequence encoding the Vα or Vγ region comprises SEQ ID NO:301, and the nucleotide sequence encoding the Vβ or Vδ region comprises SEQ ID NO:321; the nucleotide sequence encoding the Vα or Vγ region comprises SEQ ID NO:302, and the nucleotide sequence encoding the Vβ or Vδ region comprises SEQ ID NO:322; the nucleotide sequence encoding the Vα or Vγ region comprises SEQ ID NO:303, and the nucleotide sequence encoding the Vβ or Vδ region comprises SEQ ID NO:323; the nucleotide sequence encoding the Vα or Vγ region comprises SEQ ID NO:304, and the nucleotide sequence encoding the Vβ or Vδ region comprises SEQ ID NO:324; the nucleotide sequence encoding the Vα or Vγ region comprises SEQ ID NO:305, and the nucleotide sequence encoding the Vβ or Vδ region comprises SEQ ID NO:325; the nucleotide sequence encoding the Vα or Vγ region comprises SEQ ID NO:306, and the nucleotide sequence encoding the Vβ or Vδ region comprises SEQ ID NO:326; the nucleotide sequence encoding the Vα or Vγ region comprises SEQ ID NO:307, and the nucleotide sequence encoding the Vβ or Vδ region comprises SEQ ID NO:327; the nucleotide sequence encoding the Vα or Vγ region comprises SEQ ID NO:308, and the nucleotide sequence encoding the Vβ or Vδ region comprises SEQ ID NO:328; the nucleotide sequence encoding the Vα or Vγ region comprises SEQ ID NO:309, and the nucleotide sequence encoding the Vβ or Vδ region comprises SEQ ID NO:329; the nucleotide sequence encoding the Vα or Vγ region comprises SEQ ID NO:310, and the nucleotide sequence encoding the Vβ or Vδ region comprises SEQ ID NO:330; the nucleotide sequence encoding the Vα or Vγ region comprises SEQ ID NO:311, and the nucleotide sequence encoding the Vβ or Vδ region comprises SEQ ID NO:331; the nucleotide sequence encoding the Vα or Vγ region comprises SEQ ID NO:312, and the nucleotide sequence encoding the Vβ or Vδ region comprises SEQ ID NO:332; the nucleotide sequence encoding the Vα or Vγ region comprises SEQ ID NO:313, and the nucleotide sequence encoding the Vβ or Vδ region comprises SEQ ID NO:333; the nucleotide sequence encoding the Vα or Vγ region comprises SEQ ID NO:314, and the nucleotide sequence encoding the Vβ or Vδ region comprises SEQ ID NO:334; the nucleotide sequence encoding the Vα or Vγ region comprises SEQ ID NO:315, and the nucleotide sequence encoding the Vβ or Vδ region comprises SEQ ID NO:335; the nucleotide sequence encoding the Vα or Vγ region comprises SEQ ID NO:316, and the nucleotide sequence encoding the Vβ or Vδ region comprises SEQ ID NO:336; the nucleotide sequence encoding the Vα or Vγ region comprises SEQ ID NO:317, and the nucleotide sequence encoding the Vβ or Vδ region comprises SEQ ID NO:337; the nucleotide sequence encoding the Vα or Vγ region comprises SEQ ID NO:318, and the nucleotide sequence encoding the Vβ or Vδ region comprises SEQ ID NO:338; the nucleotide sequence encoding the Vα or Vγ region comprises SEQ ID NO:319, and the nucleotide sequence encoding the Vβ or Vδ region comprises SEQ ID NO:339; or the nucleotide sequence encoding the Vα or Vγ region comprises SEQ ID NO:320, and the nucleotide sequence encoding the Vβ or Vδ region comprises SEQ ID NO:340. In some of any of the embodiments provided herein, the polynucleotide comprises a nucleotide sequence encoding an alpha chain and a nucleotide sequence encoding a beta chain; or a nucleotide sequence encoding a gamma chain and a nucleotide sequence encoding a delta chain; wherein: the nucleotide sequence encoding the alpha or gamma chain comprises SEQ ID NO:8 or a sequence that has at least 90% sequence identity thereto, and the nucleotide sequence encoding the beta or delta chain comprises SEQ ID NO:16 or a sequence that has at least 90% sequence identity thereto; the nucleotide sequence encoding the alpha or gamma chain comprises SEQ ID NO:26 or a sequence that has at least 90% sequence identity thereto, and the nucleotide sequence encoding the beta or delta chain comprises SEQ ID NO:32 or a sequence that has at least 90% sequence identity thereto; the nucleotide sequence encoding the alpha or gamma chain comprises SEQ ID NO:42 or a sequence that has at least 90% sequence identity thereto, and the nucleotide sequence encoding the beta or delta chain comprises SEQ ID NO:48 or a sequence that has at least 90% sequence identity thereto; the nucleotide sequence encoding the alpha or gamma chain comprises SEQ ID NO:56 or a sequence that has at least 90% sequence identity thereto, and the nucleotide sequence encoding the beta or delta chain comprises SEQ ID NO:62 or a sequence that has at least 90% sequence identity thereto; the nucleotide sequence encoding the alpha or gamma chain comprises SEQ ID NO:70 or a sequence that has at least 90% sequence identity thereto, and the nucleotide sequence encoding the beta or delta chain comprises SEQ ID NO:73 or a sequence that has at least 90% sequence identity thereto; the nucleotide sequence encoding the alpha or gamma chain comprises SEQ ID NO:83 or a sequence that has at least 90% sequence identity thereto, and the nucleotide sequence encoding the beta or delta chain comprises SEQ ID NO:89 or a sequence that has at least 90% sequence identity thereto; the nucleotide sequence encoding the alpha or gamma chain comprises SEQ ID NO:97 or a sequence that has at least 90% sequence identity thereto, and the nucleotide sequence encoding the beta or delta chain comprises SEQ ID NO: 103 or a sequence that has at least 90% sequence identity thereto; the nucleotide sequence encoding the alpha or gamma chain comprises SEQ ID NO: 111 or a sequence that has at least 90% sequence identity thereto, and the nucleotide sequence encoding the beta or delta chain comprises SEQ ID NO:117 or a sequence that has at least 90% sequence identity thereto; the nucleotide sequence encoding the alpha or gamma chain comprises SEQ ID NO:125 or a sequence that has at least 90% sequence identity thereto, and the nucleotide sequence encoding the beta or delta chain comprises SEQ ID NO: 131 or a sequence that has at least 90% sequence identity thereto; the nucleotide sequence encoding the alpha or gamma chain comprises SEQ ID NO:141 or a sequence that has at least 90% sequence identity thereto, and the nucleotide sequence encoding the beta or delta chain comprises SEQ ID NO: 147 or a sequence that has at least 90% sequence identity thereto; the nucleotide sequence encoding the alpha or gamma chain comprises SEQ ID NO: 157 or a sequence that has at least 90% sequence identity thereto, and the nucleotide sequence encoding the beta or delta chain comprises SEQ ID NO: 163 or a sequence that has at least 90% sequence identity thereto; the nucleotide sequence encoding the alpha or gamma chain comprises SEQ ID NO:171 or a sequence that has at least 90% sequence identity thereto, and the nucleotide sequence encoding the beta or delta chain comprises SEQ ID NO: 177 or a sequence that has at least 90% sequence identity thereto; the nucleotide sequence encoding the alpha or gamma chain comprises SEQ ID NO:185 or a sequence that has at least 90% sequence identity thereto, and the nucleotide sequence encoding the beta or delta chain comprises SEQ ID NO: 191 or a sequence that has at least 90% sequence identity thereto; the nucleotide sequence encoding the alpha or gamma chain comprises SEQ ID NO: 199 or a sequence that has at least 90% sequence identity thereto, and the nucleotide sequence encoding the beta or delta chain comprises SEQ ID NO:205 or a sequence that has at least 90% sequence identity thereto; the nucleotide sequence encoding the alpha or gamma chain comprises SEQ ID NO:213 or a sequence that has at least 90% sequence identity thereto, and the nucleotide sequence encoding the beta or delta chain comprises SEQ ID NO:219 or a sequence that has at least 90% sequence identity thereto; the nucleotide sequence encoding the alpha or gamma chain comprises SEQ ID NO:229 or a sequence that has at least 90% sequence identity thereto, and the nucleotide sequence encoding the beta or delta chain comprises SEQ ID NO:235 or a sequence that has at least 90% sequence identity thereto; the nucleotide sequence encoding the alpha or gamma chain comprises SEQ ID NO:243 or a sequence that has at least 90% sequence identity thereto, and the nucleotide sequence encoding the beta or delta chain comprises SEQ ID NO:249 or a sequence that has at least 90% sequence identity thereto; the nucleotide sequence encoding the alpha or gamma chain comprises SEQ ID NO:257 or a sequence that has at least 90% sequence identity thereto, and the nucleotide sequence encoding the beta or delta chain comprises SEQ ID NO:263 or a sequence that has at least 90% sequence identity thereto; the nucleotide sequence encoding the alpha or gamma chain comprises SEQ ID NO:273 or a sequence that has at least 90% sequence identity thereto, and the nucleotide sequence encoding the beta or delta chain comprises SEQ ID NO:275 or a sequence that has at least 90% sequence identity thereto; or the nucleotide sequence encoding the alpha or gamma chain comprises SEQ ID NO:283 or a sequence that has at least 90% sequence identity thereto, and the nucleotide sequence encoding the beta or delta chain comprises SEQ ID NO:289 or a sequence that has at least 90% sequence identity thereto.

In some of any of the embodiments provided herein, the nucleotide sequence encoding the alpha or gamma chain comprises SEQ ID NO:8, and the nucleotide sequence encoding the beta or delta chain comprises SEQ ID NO:16; the nucleotide sequence encoding the alpha or gamma chain comprises SEQ ID NO:26, and the nucleotide sequence encoding the beta or delta chain comprises SEQ ID NO:32; the nucleotide sequence encoding the alpha or gamma chain comprises SEQ ID NO:42, and the nucleotide sequence encoding the beta or delta chain comprises SEQ ID NO:48; the nucleotide sequence encoding the alpha or gamma chain comprises SEQ ID NO:56, and the nucleotide sequence encoding the beta or delta chain comprises SEQ ID NO:62; the nucleotide sequence encoding the alpha or gamma chain comprises SEQ ID NO:70, and the nucleotide sequence encoding the beta or delta chain comprises SEQ ID NO:73; the nucleotide sequence encoding the alpha or gamma chain comprises SEQ ID NO:83, and the nucleotide sequence encoding the beta or delta chain comprises SEQ ID NO:89; the nucleotide sequence encoding the alpha or gamma chain comprises SEQ ID NO:97, and the nucleotide sequence encoding the beta or delta chain comprises SEQ ID NO:103; the nucleotide sequence encoding the alpha or gamma chain comprises SEQ ID NO:111, and the nucleotide sequence encoding the beta or delta chain comprises SEQ ID NO:117; the nucleotide sequence encoding the alpha or gamma chain comprises SEQ ID NO:125, and the nucleotide sequence encoding the beta or delta chain comprises SEQ ID NO:131; the nucleotide sequence encoding the alpha or gamma chain comprises SEQ ID NO:141, and the nucleotide sequence encoding the beta or delta chain comprises SEQ ID NO:147; the nucleotide sequence encoding the alpha or gamma chain comprises SEQ ID NO:157, and the nucleotide sequence encoding the beta or delta chain comprises SEQ ID NO:163; the nucleotide sequence encoding the alpha or gamma chain comprises SEQ ID NO:171, and the nucleotide sequence encoding the beta or delta chain comprises SEQ ID NO:177; the nucleotide sequence encoding the alpha or gamma chain comprises SEQ ID NO:185, and the nucleotide sequence encoding the beta or delta chain comprises SEQ ID NO:191; the nucleotide sequence encoding the alpha or gamma chain comprises SEQ ID NO:199, and the nucleotide sequence encoding the beta or delta chain comprises SEQ ID NO:205; the nucleotide sequence encoding the alpha or gamma chain comprises SEQ ID NO:213, and the nucleotide sequence encoding the beta or delta chain comprises SEQ ID NO:219; the nucleotide sequence encoding the alpha or gamma chain comprises SEQ ID NO:229, and the nucleotide sequence encoding the beta or delta chain comprises SEQ ID NO:235; the nucleotide sequence encoding the alpha or gamma chain comprises SEQ ID NO:243, and the nucleotide sequence encoding the beta or delta chain comprises SEQ ID NO:249; the nucleotide sequence encoding the alpha or gamma chain comprises SEQ ID NO:257, and the nucleotide sequence encoding the beta or delta chain comprises SEQ ID NO:263; the nucleotide sequence encoding the alpha or gamma chain comprises SEQ ID NO:273, and the nucleotide sequence encoding the beta or delta chain comprises SEQ ID NO:275; or the nucleotide sequence encoding the alpha or gamma chain comprises SEQ ID NO:283, and the nucleotide sequence encoding the beta or delta chain comprises SEQ ID NO:289.

In some of any of the embodiments provided herein, the nucleotide sequence encoding the alpha chain and the nucleotide sequence encoding the beta chain are separated by a peptide sequence that causes ribosome skipping. In some of any of the embodiments provided herein, the peptide that causes ribosome skipping is a P2A peptide. In some of any of the embodiments provided herein, the P2A peptide comprises SEQ ID NO:352. In some of any of the embodiments provided herein, the sequence encoding the P2A peptide is set forth in SEQ ID NO:351.

In some of any of the embodiments provided herein, the nucleotide sequence encodes SEQ ID NO:18; the nucleotide sequence encodes SEQ ID NO:34; the nucleotide sequence encodes SEQ ID NO:50; the nucleotide sequence encodes SEQ ID NO:64; the nucleotide sequence encodes SEQ ID NO:75; the nucleotide sequence encodes SEQ ID NO:91; the nucleotide sequence encodes SEQ ID NO:105; the nucleotide sequence encodes SEQ ID NO:119; the nucleotide sequence encodes SEQ ID NO: 133; the nucleotide sequence encodes SEQ ID NO: 149; the nucleotide sequence encodes SEQ ID NO: 165; the nucleotide sequence encodes SEQ ID NO: 179; the nucleotide sequence encodes SEQ ID NO: 193; the nucleotide sequence encodes SEQ ID NO:207; the nucleotide sequence encodes SEQ ID NO:221; the nucleotide sequence encodes SEQ ID NO:237; the nucleotide sequence encodes SEQ ID NO:251; the nucleotide sequence encodes SEQ ID NO:265; the nucleotide sequence encodes SEQ ID NO:277; the nucleotide sequence encodes SEQ ID NO:291; the nucleotide sequence encodes SEQ ID NO:367; the nucleotide sequence encodes SEQ ID NO:377; the nucleotide sequence encodes SEQ ID NO:387; the nucleotide sequence encodes SEQ ID NO:397; the nucleotide sequence encodes SEQ ID NO:407; the nucleotide sequence encodes SEQ ID NO:417; the nucleotide sequence encodes SEQ ID NO:427; the nucleotide sequence encodes SEQ ID NO:437; the nucleotide sequence encodes SEQ ID NO:447; the nucleotide sequence encodes SEQ ID NO:457; or the nucleotide sequence encodes SEQ ID NO:487.

In some of any of the embodiments provided herein, the nucleotide sequence comprises SEQ ID NO:17; the nucleotide sequence comprises SEQ ID NO:33; the nucleotide sequence comprises SEQ ID NO:49; the nucleotide sequence comprises SEQ ID NO:63; the nucleotide sequence comprises SEQ ID NO:74; the nucleotide sequence comprises SEQ ID NO:90; the nucleotide sequence comprises SEQ ID NO: 104; the nucleotide sequence comprises SEQ ID NO: 118; the nucleotide sequence comprises SEQ ID NO:132; the nucleotide sequence comprises SEQ ID NO:148; the nucleotide sequence comprises SEQ ID NO:164; the nucleotide sequence comprises SEQ ID NO:178; the nucleotide sequence comprises SEQ ID NO:192; the nucleotide sequence comprises SEQ ID NO:206; the nucleotide sequence comprises SEQ ID NO:220; the nucleotide sequence comprises SEQ ID NO:236; the nucleotide sequence comprises SEQ ID NO:250; the nucleotide sequence comprises SEQ ID NO:264; the nucleotide sequence comprises SEQ ID NO:276; the nucleotide sequence comprises SEQ ID NO:290; the nucleotide sequence comprises SEQ ID NO:368; the nucleotide sequence comprises SEQ ID NO:378; the nucleotide sequence comprises SEQ ID NO:388; the nucleotide sequence comprises SEQ ID NO:398; the nucleotide sequence comprises SEQ ID NO:408; the nucleotide sequence comprises SEQ ID NO:418; the nucleotide sequence comprises SEQ ID NO:428; the nucleotide sequence comprises SEQ ID NO:438; the nucleotide sequence comprises SEQ ID NO:448; the nucleotide sequence comprises SEQ ID NO:458; or the nucleotide sequence comprises SEQ ID NO:487.

Also provided herein are vectors comprising any of the polynucleotides provided herein. In some of any of the embodiments provided herein, the vector is a viral vector. In some of any of the embodiments provided herein, the viral vector is a lentiviral vector.

Also provided herein are engineered cells comprising any of the TCRs or antigen-binding fragment thereof provided herein. Also provided herein are engineered cells comprising any of the polynucleotides provided herein or any of the vectors provided herein. In some of any of the embodiments provided herein, the TCR or antigen-binding fragment thereof is heterologous to the cell. In some of any of the embodiments provided herein, the engineered cell is a cell line. In some of any of the embodiments provided herein, the engineered cell is a primary cell obtained from a subject. In some of any of the embodiments provided herein, the engineered cell is a T cell.

Also provided herein are methods for producing an engineered cell that involve introducing any of the polynucleotides provided herein or any of the vectors provided herein into a cell to form the engineered cell.

Also provided herein are compositions comprising any of the TCRs or antigen-binding fragment thereof provided herein, any of the polynucleotides provided herein, any of the vectors provided herein, or any of the engineered cells provided herein. In some of any of the embodiments provided herein, the compositions also include a pharmaceutically acceptable excipient.

Also provided herein are methods for identifying a TCR targeting a hematopoietically restricted minor histocompatibility antigen (miHA), that involve identifying a functional TCR that recognizes a hematopoietically-restricted miHA, among a plurality of functional TCRs, wherein said plurality of functional TCRs are encoded by a plurality of functional TCR-encoding nucleic acid vectors generated by a high-throughput nucleic acid amplification and assembly method using nucleic acid obtained from a single T cell among a plurality of T cells; wherein said plurality of T cells is from a human female donor that is pregnant with or has been pregnant with a fetus with a mismatched or immunogenic hematopoietically restricted miHA.

Also provided herein are methods for identifying a TCR targeting a hematopoietically restricted miHA, that involve: (i) generating a plurality of functional TCR-encoding nucleic acid vectors by a high-throughput nucleic acid amplification and assembly method using nucleic acid obtained from a single T cell among a plurality of T cells; wherein said T cell is from a human female donor that is pregnant with or has been pregnant with a fetus with a mismatched or immunogenic hematopoietically restricted miHA; and (ii) identifying a functional TCR that recognizes a hematopoietically-restricted miHA, among a plurality functional TCRs encoded by the plurality of functional TCR-encoding nucleic acid vectors.

In some of any of the embodiments provided herein, the hematopoietically restricted miHA is a minor histocompatibility antigen HA-1. In some of any of the embodiments provided herein, the identified functional TCR recognizes a peptide epitope of a minor histocompatibility antigen HA-1 in the context of an MHC molecule. In some of any of the embodiments provided herein, the MHC molecule is a human leukocyte antigens (HLA)-A molecule. In some of any of the embodiments provided herein, the HLA-A molecule is of serotype HLA-A*02:01. In some of any of the embodiments provided herein, the HLA-A molecule is of serotype HLA-A*02:06. In some of any of the embodiments provided herein, the peptide epitope of HA-1 is set forth in SEQ ID NO:354.

In some of any of the embodiments provided herein, the T cell from the human female donor is cultured under conditions for cell expansion of the T cell prior to the generating of the plurality of functional TCR-encoding nucleic acid vectors.

In some of any of the embodiments provided herein, the T cell from the human female donor is not cultured under conditions for cell expansion of the T cell prior to the generating of the plurality of functional TCR-encoding nucleic acid vectors.

In some of any of the embodiments provided herein, the high-throughput nucleic acid amplification and assembly involves: (1) amplifying a first amplification product and a second amplification product from complementary DNA (cDNA) generated from RNA obtained from the single T cell among the plurality of T cells sorted into each of a plurality of separate locations of a device, wherein: said first amplification product comprises a nucleotide sequence encoding a full-length Vα region or a full-length Vγ region of a TCR, and said second amplification product comprises a nucleotide sequence encoding a full-length Vβ region or a full-length Vδ region of a TCR; and (2) assembling said first amplification product and said second amplification product from each of said plurality of separate locations into a nucleic acid vector to obtain an assembled nucleic acid vector comprising a nucleotide sequence encoding a functional TCR for each of said plurality of separate locations; and said functional TCR comprises (i) a full-length Vα region and a full-length Vβ region from said single T cell or (ii) a full-length Vγ region and a full-length Vδ region from said single T cell.

Also provided herein are engineered cells comprising the TCR identified by any of the methods described herein. Also provided herein are compositions comprising any of the engineered cells provided herein. In some of any of the embodiments provided herein, the composition also comprises a pharmaceutically acceptable excipient.

Also provided herein are methods of treatment that involve administering any of the TCRs or antigen-binding fragment thereof provided herein, any of the polynucleotides provided herein, any of the vectors provided herein, any of the engineered cells provided herein, or any of the compositions provided herein, to a subject having a disease or a disorder. Also provided herein are any of the TCRs or antigen-binding fragment thereof provided herein, any of the polynucleotides provided herein, any of the vectors provided herein, any of the engineered cells provided herein, or any of the compositions provided herein, for use in the treatment of a disease or a disorder in a subject. Also provided herein are uses of any of the TCRs or antigen-binding fragment thereof provided herein, any of the polynucleotides provided herein, any of the vectors provided herein, any of the engineered cells provided herein, or any of the compositions provided herein in the manufacture of a medicament for the treatment of a disease or a disorder in a subject. Also provided herein are uses of any of the TCRs or antigen-binding fragment thereof provided herein, any of the polynucleotides provided herein, any of the vectors provided herein, any of the engineered cells provided herein, or any of the compositions provided herein, for the treatment of a disease or a disorder in a subject.

In some of any of the embodiments provided herein, the subject is eligible for or is to receive an allogeneic hematopoietic stem cell transplantation (HSCT). In some of any of the embodiments provided herein, the subject has or has been diagnosed with a malignant hematologic disorder. In some of any of the embodiments provided herein, the subject has or has been diagnosed with acute myeloid leukemia (AML), myelodysplastic syndrome (MDS), or acute lymphoblastic leukemia (ALL). In some of any of the embodiments provided herein, the subject has or has been diagnosed with a liquid tumor, a hematopoietic tumor, a lymphoma, or chronic myeloid leukemia CML. In some of any of the embodiments provided herein, administration of the engineered cell or the composition induces or enhances cells death of cells associated with the malignant hematologic disorder, or induces or enhances a graft versus leukemia effect (GVL) in the subject.

### BRIEF DESCRIPTION OF THE DRAWINGS

FIG. 1 shows FACS analysis of expanded donor cells, PBMCs alone (left panel) or PBMCs co-cultured with B cells from the same donor (B-APCs; right panel), double-stained with miHA-1 dextramers labeled with either FITC or APC.

FIG. 2 shows FACS plot analysis of controls used to screen for miHA-1 specific TCRs. An empty vector and a non-HA-1 specific TCR served as negative controls, and an exemplary HA-1 targeting TCR was used as a positive control.

FIG. 3 shows a plot of TRAV, TRBV expression and % of CD3 as assessed by FACS, in 701 TCR clones transiently expressed in HEK 293 cells. Clones that exhibited higher surface TCR expression than the positive control are shown in blue, clones that exhibited lower surface TCR expression than the negative control are shown in yellow, and those in between the two controls are shown in green.

FIG. 4A shows an FACS-based assessment of individual TCRs expressed in HEK 293 cells to determine transfection efficiency, as indicated by mCherry and IP26 staining. FIG. 4B shows FACS analysis of individual TCRs expressed in HEK 293 stained with HA-1 “H” dextramer.

FIG. 5 shows TCR expression in relation to beta chain variants (TRBV), following sequencing of amplified TRBVs in a screen of 704 receptors.

FIG. 6 shows the distribution of non-singleton clones between unexpanded and expanded cultures, including those expanded in the presence of the naked HA-1 peptide or HA-1 presented by donor B cells.

FIG. 7 shows the percentage of CD3 cells that were stained positive for the HA-1 “H” dextramer, among 704 clones obtained from the parous woman and re-expressed in reporter cells.

FIG. 8 shows an EC₅₀ assessment from a CD69 activation study of reporter cells expressing one of 16 exemplary anti-HA-1 TCRs.

FIG. 9 shows an assessment of TCR binding in the presence of either the HA-1 “H” peptide or the HA-1 “R” peptide.

FIG. 10 shows an IL-2 secretion assay, wherein the functionality of Jurkat T cells transduced with HA-1 targeting TCRs was assessed in the presence of APCs presenting the target “H” peptide.

FIG. 11 shows an IL-2 secretion assay, which assessed the functionality of Jurkat T cells transduced with HA-1 targeting TCRs in response to APCs presenting either the HA-1 “H” or the HA-1 “R” peptide.

FIG. 12 shows the results of an EC₅₀ assessment from a CD69 activation study of Jurkat T cells expressing various TCR candidates after an overnight incubation with A*0201 LCLs and increasing concentrations of the HA-1 “H” peptide.

FIG. 13A shows a representative EC₅₀ assessment, wherein Jurkat T cells expressing an exemplary HA-1 specific TCR were assessed for CD69 activation in response to increasing concentrations of HA-1 peptide. FIG. 13B shows the results of experiments to determine the EC₅₀ of an exemplary TCR in the presence of increasing concentrations of HA-1.

FIG. 14 shows a comparison of the EC₅₀ determined for an exemplary TCR with reconstructed CD69 activation data from known HA-1 specific TCRs.

FIG. 15 shows CD69 activation of exemplary anti-HA-1 TCR expressing cells in response to different HA-1 peptide alleles presented by HLA-A*02:01 LCLs and non-HLA-A*02:01 LCLs.

FIG. 16 shows A*0201/HA-1 dextramer staining of an exemplary HA-1 TCR expressed in human primary T cells.

FIG. 17 shows flow cytometry plots of cell populations in a cell killing assay, wherein LCLs loaded with the HA-1 “H” or “R” were labeled with CFSE to indicate killing and target specificity by HA-1 specific TCRs expressed in primary human cells.

FIG. 18A shows cell counts of HA-1 “H”- and HA-1 “R”—loaded LCLs following exposure to non-transduced T cells at increasing effector to target (E:T) ratios. FIG. 18B shows cell counts of HA-1 “H”- and HA-1 “R”—loaded LCLs following exposure to HA-1 specific TCRs expressed in human primary T cells at increasing effector to target (E:T) ratios.

### DETAILED DESCRIPTION

Provided herein are TCRs, such as recombinant TCRs, such as those that bind or recognize a peptide epitope associated with a hematopoietically-restricted minor histocompatibility antigen, e.g., HA-1, such as a peptide epitope expressed on the surface of a cell in the context of an MHC molecule. Among the provided embodiments are approaches useful in the treatment of such diseases and conditions and/or for targeting cell types, such as cancer cells or cells associated with a hematological ailment. In some embodiments, the provided TCRs and antigen-binding fragments thereof, bind or recognize a peptide epitope of HA-1, in the context of an MHC molecule.

Also provided herein are nucleic acid molecules encoding the TCRs, engineered cells containing the TCRs, compositions containing the TCRs or cells, and methods of treatment or uses, such as therapeutic uses, involving administering such TCRs, engineered cells or compositions, and uses of such TCRs, cells or compositions. In some aspects, engineered cells that express a provided TCR or antigen binding fragment thereof, exhibit cytotoxic activity against target cells expressing the peptide epitope, such as cancer cells or cells associated with a hematological ailment. Also provided herein are methods for identifying a TCR targeting a hematopoietically restricted miHA.

Allogeneic Stem Cell Transplantation (Allo-SCT) can be a curative therapy for patients with hematologic malignancies as well as for patients with nonmalignant but medically serious conditions such as hemoglobinopathies, thalassemias and autoimmune diseases. AlloSCT can also be used to create tolerance to transplanted solid organs. Mature αβ T cells contained in the donor allograft play important roles and can be considered in two broad classes. One class promotes the reconstitution of anti-pathogen immunity, especially through the transfer of memory T cells. A second class of T cells, called alloreactive T cells, recognizes the patient as “non-self”. When alloSCT is used for the treatment of hematological malignancies, alloreactive donor T cells can kill malignant cells, thereby mediating a graft-versus-leukemia (GVL) effect. However, they can also cause graft-versus-host disease (GVHD), wherein alloreactive T cells attack healthy host tissues, including, e.g., the skin, bowel and liver.

In a human leukocyte antigen (HLA) matched alloSCT, alloreactive T cells target miHAs, the peptide products of coding polymorphisms that distinguish recipients from donors. Importantly, alloreactive CD8+ T cells that target miHAs with expression limited to hematopoietic cells are unlikely to cause GVHD. Administering anti-miHA T cells could minimize the risk of widespread toxicity without compromising therapeutic efficacy in the context of augmenting alloSCT or as a standalone therapy, among other strategies.

Cell therapies (including those involving the administration of cells expressing recombinant receptors or TCRs specific for a disease or disorder of interest, such as a recombinant TCR and/or other recombinant antigen receptors), as well as other adoptive immune cell and adoptive T cell therapies can be effective in the treatment of diseases and disorders. In certain contexts, available approaches to adoptive cell therapy may not always be entirely satisfactory. In some contexts, optimal efficacy can depend on the ability of the administered cells to express the recombinant receptor, and for the recombinant receptor to recognize and bind to a target, e.g., target antigen, such as peptide epitopes of HA-1, within the subject, for example, based on the affinity of the antigen-binding domain of the TCR to its target antigen. In some cases, consistency and/or efficiency of expression of the recombinant receptor, and activity of the receptor is limited in certain cells or certain cell populations of available therapeutic approaches.

In some aspects, development of a humanized and/or fully human recombinant TCR presents technical challenges. For example, in some aspects, a humanized and/or a fully human recombinant TCR, when engineered into a human T cell, competes with endogenous TCR complexes and/or can form mispairings with endogenous TCR chains, which may, in certain aspects, reduce recombinant TCR signaling, activity, and/or expression, and ultimately result in reduced activity of the engineered cells. For example, in some cases, suboptimal expression of an engineered or recombinant TCR can occur due to competition with an endogenous TCR and/or with TCRs having mispaired chains, for signaling molecules and/or domains such as the invariant CD3 signaling molecules (e.g., availability of co-expressed CD3 8, &, γ and/or (chains) that are involved in permitting expression of the complex on the cell surface. In some aspects, available CD3 molecules can limit the expression and function of the TCRs in the cells.

In some aspects, the provided embodiments are based on observations of improved affinity, expression or activity of an exemplary fully human recombinant TCR, such as certain provided TCRs specific to HA-1, even at a low effector to target (E:T) ratio. The activity of the engineered T cells expressing a recombinant TCR, e.g., cytokine secretion and/or cytolytic activity, in some cases may be limited when fewer engineered T cells are present compared to the target cells. In some aspects, such improvements in activity, particularly at a low E:T ratio and using fully human sequences, are advantageous in improving the efficacy of the therapy.

In some cases, certain available approaches to obtain antigen-specific recombinant receptors, such as recombinant TCRs, can result in recombinant receptors that exhibit cross reactivity to a different, non-target antigen (see, e.g., Cameron et al., (2013) Science Translational Medicine, 5(197): 197ra103). In some aspects, the provided embodiments are based on observations that as described herein, for example, that certain provided TCRs specific to a particular immunogenic HA-1 peptide presented by HLA subtype A*0201, do not show cross reactivity to cells expressing other peptide antigens or alloreactivity to other HLA subtypes. The provided TCRs thus exhibit improved expression and activity, with minimal risk of cross reactivity to other antigens, such as non-target antigens, that can be present in the subject, or peptide epitopes presented via non-target HLA subtypes.

In some aspects, therapeutic approaches using such TCRs, for example adoptive cell therapy with engineered human T cells expressing the provided recombinant TCRs, can ultimately result in high efficacy, for example, by improving the GVL effect. In some contexts, the provided embodiments, including the TCRs, polynucleotides encoding such TCRs, engineered cells and cell compositions, can provide various advantages over available therapies with TCRs, to improve the activity of the recombinant receptors and response to adoptive cell therapies targeting cancer cells and cells associated with hematological ailments.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. T Cell Receptors Targeting Histocompatibility Antigen 1 (Ha-1)

Provided herein are TCRs, such as those that bind or recognize a peptide epitope associated with a hematopoietically restricted miHA, such as a peptide epitope expressed on the surface of a cancer cell and/or a cell associated with a hematological ailment, in the context of an MHC molecule. In some embodiments, the provided TCRs bind or recognize a peptide epitope of miHA HA-1, in the context of an MHC molecule. Such TCRs and antigen-binding fragments exhibit antigenic specificity for binding or recognizing such peptide epitopes. Also provided in some embodiments are nucleic acid molecules encoding the TCRs, engineered cells expressing the TCRs, compositions and methods of treatment involving administering such TCRs, engineered cells or compositions. In some aspects, engineered cells that express a provided TCR or antigen-binding fragment, exhibit cytotoxic activity against target cells expressing the peptide epitope, such as cancer cells or cells that are associated with a hematological ailment.

### A. Allogeneic Stem Cell Transplantation

AlloSCT is a curative treatment option for patients with hematologic malignancies. In an alloSCT, patients receive a conditioning regimen, consisting of chemotherapy sometimes with radiation therapy, which facilitates the transplant by killing some residual malignant cells, by creating space in the recipient's bone marrow for donor stem cell engraftment, and by killing patient immune cells that can mediate donor allograft rejection.

Mature alpha/beta donor T cells contained in the donor graft play important roles and can be considered in two broad classes. One class promotes the reconstitution of anti-pathogen immunity, especially through the transfer of memory T cells. A second class of T cells recognizes the patient as “nonself.” These so called “alloreactive” T cells have both positive and detrimental effects. A critical benefit of these cells is that they can kill recipient malignant cells mediating the GVL effect. Alloreactive T cells can also kill normal patient or host hematopoietic and immune cells, which both creates space for engrafting cells and reduces immunologic rejection of the donor cells. However, donor T cells can also attack normal recipient tissues, causing GVHD. Therefore, all patients receive some type of systemic immunosuppression to reduce the frequency and severity of GVHD.

Despite the GVL effect, relapsed malignancy is the largest single cause of treatment failure and death in recipients of an alloSCT in treatment of a blood neoplasm. There is good reason to believe that relapse can be reduced by engineering a more effective alloreactive T cell response. GVHD and the consequences of systemic immunosuppression (such as infection) are the other major causes of morbidity and mortality. These too could be mitigated if the allo-response was better engineered to focus on hematopoietic cells and not normal host tissues. Despite these limitations, alloSCT is the worldwide standard of care for patients with moderate to high-risk hematologic malignancies supported by data from multiple sources that support higher rates of survival with than without a transplant.

In HLA-matched alloSCT, alloreactive donor T cells target miHAs expressed in the recipient. MiHAs are the peptide products of coding polymorphisms that distinguish recipients from donors. These polymorphisms are present in stable known frequencies in the population and are inherited by Mendelian genetics. Some of the genes that encode miHAs are similarly expressed in a wide spectrum of tissues, whereas others are relatively restricted to hematopoietic cells. As currently practiced, there is no control over which miHAs will be targeted in an alloSCT. Because T cell responses against miHAs expressed on normal tissues are nearly always generated, severe GVHD is a major risk, and therefore immunosuppression is required. In contrast, miHAs with expression relatively limited to hematopoietic cells are considered ideal targets for immunotherapy with donor derived CD8+ T cells, in conjunction with an alloSCT. CD8+ T cells that target such antigens can kill recipient malignant blood cells mediating the GVL effect. T cells that target hematopoietically restricted antigen can mediate graft-versus-leukemia and promote engraftment with a low risk for graft-vs-host disease. They can also kill nonmalignant recipient hematopoietic cells, including immune cells, thereby promoting engraftment and reducing the risk of immunologic rejection. Interestingly, CD8+ T cells that target hematopoietically-restricted miHAs have a low risk of causing GVHD.

CD8+ T cells recognize their targets through their antigen receptors (TCRs). The process that generates these receptors creates a highly diverse repertoire of unique TCRs with each person estimated to contain T cells expressing more than 107 unique receptors. This diversity allows people to respond not only to a wide range of pathogens but also to miHAs. Unlike antibodies, which bind to intact proteins, TCRs recognize short peptides, usually about 8-12 amino acids in length, embedded in the surface of MHC molecules, which are expressed on the surface of cells.

An advantage of this system of antigen detection is that T cells can recognize peptides derived from any protein, even those that are not expressed on the cell surface. Through evolution, MHC molecules have become diverse in the population with most of the variation being in the parts of the MHC molecule that bind peptide, and which present or display the peptide to the TCR. This allows for a large diversity of peptides that can be presented to T cells by MHC molecules with preference or restriction of certain peptides to specific MHC molecules. A consequence of this is that each miHA is generally restricted to a single MHC type. Importantly, over the last several decades, more than 50 relatively hematopoietically-restricted miHAs have been identified, a number more than sufficient such that nearly every donor/recipient combination, regardless of MHC type, would have a targetable hematopoietically-restricted miHA.

### B. Histocompatibility Antigen 1 (HA-1)

HA-1 is a hematopoietically restricted miHA that has been found on Hofbauer and trophoblast cells in the human placenta, and is important for bone marrow transplantation outcomes. The antigenic peptide that arises from HA-1 results from a single nucleotide difference between the non-immunogenic (“R peptide”) comprising the amino acid sequence VLRDDLLEA (SEQ ID NO:355) and the immunogenic (“H peptide”) comprising the amino acid sequence VLHDDLLEA (SEQ ID NO:354). The immunogenic peptide can be presented in the context of at least four different Class I MHC molecules, including HLA-A*0201, A*0206, B*60, and B*40012. As a result of the immunogenic single nucleotide polymorphism (SNP), the binding affinity of the HA-1 H peptide to the HLA-A*0201 peptide binding groove on antigen presenting cells (APCs) is increased, thus leading to an immunogenic peptide that can be recognized by HLA-A*0201 restricted T cells.

The difference in immunogenicity of these peptides could be due to various factors. For example, the proteasomal cleavage and transport of the two peptides into the endoplasmic reticulum via TAP is similar, and both variants can bind to the HLA-restricting allele. However, the R peptide has lower affinity and less stable binding to HLA-A*0201 likely related to the relatively large size of the arginine residue that results in steric and electrostatic hindrance with HLA-A*0201 D pocket residues. Differences in both MHC molecule and TCR binding can account for the immunogenicity of the HA-1 H peptide.

In some examples, the miHA HA-1 H peptide (VLHDDLLEA; SEQ ID NO:354) is targeted, and not the non-immunogenic R peptide (VLRDDLLEA; SEQ ID NO:355). HA-1 is ideal in that its expression is limited to hematopoietic cells, presented (or “restricted”) by the most common human HLA type and has an allele frequency of about 50%. See de Bueger et al. (1992) J Immunol Baltim Md 1950 149(5): 1788-1794 and Wilke et al. (2003) Hematol. J. 4(5):315-320.

In some aspects, the TCR recognizes or binds an HA-1 epitope in the context of an MHC molecule, such as an MHC Class I molecule. In some aspects, the MHC Class I molecule is an HLA-A2 molecule, including any one or more subtypes thereof, e.g., HLA-A*0201, *0202, *0203, *0206, or *0207. In some cases, there can be differences in the frequency of subtypes between different populations. For example, in some embodiments, more than 95% of the HLA-A2 positive Caucasian population is HLA-A*0201, whereas in the Chinese population the frequency has been reported to be approximately 23% HLA-A*0201, 45% HLA-A*0207, 8% HLA-A*0206, and 23% HLA-A*0203. In some embodiments, the MHC molecule is HLA-A*0201.

In some aspects, the provided TCRs or antigen-binding fragments thereof recognize or bind to an immunogenic (e.g., mismatched between donor and recipient of alloSCT) epitope or domain of HA-1, such as the immunogenic H peptide comprising the amino acid sequence VLHDDLLEA (SEQ ID NO:354). In some aspects, the provided TCRs or antigen-binding fragments thereof recognize or bind to a non-immunogenic epitope or domain of HA-1, such as the non-immunogenic R peptide comprising the amino acid sequence VLRDDLLEA (SEQ ID NO:355).

In some embodiments, the TCR, or antigen-binding fragment thereof, is isolated or purified or is recombinant. In particular embodiments, any of the provided TCRs, or antigen-binding fragments thereof, are recombinant. In some aspects, the TCR, or antigen-binding fragment thereof, is human. In some aspects, the TCR is a single chain. In other embodiments, the TCR contains two chains. In some embodiments, the TCR, or antigen-binding fragment thereof, is expressed on the surface of a cell (e.g., a T cell such as a T cell designed to lack expression of endogenous TCRs).

In some aspects, the provided TCRs have one or more specified functional features, such as binding properties, including binding to particular epitopes, and/or particular binding affinities, for example, as described herein. In some aspects, engineered cells, such as T cells, expressing the provided TCRs have one or more specified functional features, such as binding properties, including binding to particular epitopes, particular binding affinities, activation or stimulation of cell signaling, such as T cell signaling or TCR signaling, secretion of cytokines, and/or killing of target cells expressing or presenting the antigen, for example, as described herein.

In some embodiments, the provided binding molecule is a TCR or antigen-binding fragment thereof. In some embodiments, a TCR is a molecule that contains an alpha chain comprising a Vα region and a beta chain comprising a Vβ region (also known as TCRα and TCRβ, respectively) or a gamma chain comprising a Vγ region and a delta chain comprising a Vδ region (also known as TCRγ and TCRδ, respectively), or antigen-binding portions thereof, which is capable of specifically binding to an antigen, e.g., a peptide antigen or peptide epitope bound to an MHC molecule. In some embodiments, the TCR is in the αβ form (e.g., is an αβ TCR). In some embodiments, the TCR is in the γδ form (e.g., is an γδ TCR). Typically, TCRs that exist in αβ or γδ forms are generally structurally similar, but T cells expressing them may have distinct anatomical locations or functions. A TCR can be found on the surface of a cell or in soluble form. Generally, a TCR is found on the surface of T cells where it is generally responsible for recognizing antigens, such as peptides bound to MHC molecules.

In some embodiments, a TCR provided herein can be an intact or full-length TCR, such as a TCR containing a full length a chain and a full length β chain, or a TCR containing a full length γ chain and a full length δ chain. In some embodiments, an antigen-binding portion of a TCR provided herein can be less than a full-length TCR provided that it binds to a specific peptide bound in an MHC molecule, such as it binds to an MHC-peptide complex. In some cases, an antigen-binding portion or fragment of a TCR can contain only a portion of the structural domains of a full-length or intact TCR, but yet is able to bind the peptide epitope, such as MHC-peptide complex, to which the full-length TCR binds. In some cases, an antigen-binding portion contains the variable domains of a TCR, such as a Vα region and a Vβ region of a TCR, or a Vγ region and a Vδ region of a TCR provided herein provided that that antigen-binding portion is sufficient to form a binding site for binding to a specific MHC-peptide complex.

In some embodiments, the variable domains of the TCR contain CDRs, which generally are contributors to antigen recognition and binding capabilities and specificity of the peptide, MHC molecule, and/or MHC-peptide complex. In some embodiments, a CDR of a TCR or combination thereof forms all or substantially all of the antigen-binding site of a given TCR molecule. The various CDRs within a variable region of a TCR chain generally are separated by framework regions (FRs), which generally display less variability among TCRs as compared to the CDRs (see, e.g., Jores et al., Proc. Nat'l Acad. Sci. U.S.A. 87:9138, 1990; Chothia et al., EMBO J. 7:3745, 1988; see also Lefranc et al., Dev. Comp. Immunol. 27:55, 2003). In some embodiments, CDR-3 is the main CDR responsible for antigen binding or specificity, or is the most important among the three CDRs on a given TCR variable region for antigen recognition, and/or for interaction with the processed peptide portion of the peptide-MHC complex. In some contexts, CDR-1 of the alpha chain can interact with the N-terminal part of certain antigenic peptides. In some contexts, CDR-1 of the beta chain can interact with the C-terminal part of the peptide. In some contexts, CDR-2 contributes most strongly to or is the primary CDR responsible for the interaction with or recognition of the MHC portion of the MHC-peptide complex. In some embodiments, the variable region of the β-chain can contain a further hypervariable region (e.g., CDR4 or HVR4), which generally is involved in superantigen binding and not antigen recognition (Kotb (1995) Clinical Microbiology Reviews, 8:411-426).

In some embodiments, the a chain and/or the B chain of a TCR, or the γ chain and/or the δ chain of a TCR, also can contain a constant domain, a transmembrane domain and/or a short cytoplasmic tail (see, e.g., Janeway et al., Immunobiology: The Immune System in Health and Disease, 3^{rd} Ed., Current Biology Publications, p. 4:33, 1997). In some aspects, each chain (e.g. alpha or beta) of the TCR can possess one N-terminal immunoglobulin variable domain, one immunoglobulin constant domain, a transmembrane region, and a short cytoplasmic tail at the C-terminal end. In some embodiments, a TCR, for example via the cytoplasmic tail, is associated with invariant proteins of the CD3 complex involved in mediating signal transduction. In some cases, the structure allows the TCR to associate with other molecules like CD3 and subunits thereof. For example, a TCR containing constant domains with a transmembrane region may anchor the protein in the cell membrane and associate with invariant subunits of the CD3 signaling apparatus or complex. The intracellular tails of CD3 signaling subunits (e.g. CD3γ, CD3δ, CD3ε and CD3ζ chains) contain one or more immunoreceptor tyrosine-based activation motif or ITAM and generally are involved in the signaling capacity of the TCR complex.

The various domains or regions of a TCR can be identified. In some cases, the exact locus of a domain or region can vary depending on the particular structural or homology modeling or other features used to describe a particular domain. It is understood that reference to amino acids, including to a specific sequence set forth as a SEQ ID NO: used to describe domain organization of a TCR are for illustrative purposes and are not meant to limit the scope of the embodiments provided. In some cases, the specific domain (e.g. variable or constant) can be several amino acids (such as one, two, three or four) longer or shorter. In some aspects, residues of a TCR are known or can be identified according to the International Immunogenetics Information System (IMGT) numbering system (see e.g. www.imgt.org; see also, Lefranc et al. (2003) Developmental and Comparative Immunology, 27(1);55-77; and The T Cell Factsbook 2nd Edition, Lefranc and LeFranc Academic Press 2001). Using this system, CDR-1 sequences within a TCR Vα region and/or Vβ region in some cases correspond to the amino acids present between residue numbers 27-38, inclusive, CDR-2 sequences within a TCR Vα region and/or Vβ region in some cases correspond to the amino acids present between residue numbers 56-65, inclusive, and CDR-3 sequences within a TCR Vα region and/or Vβ region in some cases correspond to the amino acids present between residue numbers 105-117, inclusive.

In some embodiments, among the TCRs or antigen-binding fragments thereof provided herein are those that bind to or recognize a hematopoietically restricted minor histocompatibility antigen, such as HA-1, in the context of an MHC molecule. In some embodiments, among the TCRs or antigen-binding fragments thereof provided herein are those that recognize or bind to an immunogenic (e.g., mismatched between donor and recipient of alloSCT) epitope or domain of HA-1, such as the immunogenic H peptide comprising the amino acid sequence VLHDDLLEA (SEQ ID NO:354). In some embodiments, among the TCRs or antigen-binding fragments thereof provided herein are those that do not recognize or binds to a non-immunogenic epitope or domain of HA-1, such as the non-immunogenic R peptide comprising the amino acid sequence VLRDDLLEA (SEQ ID NO:355). In some embodiments, among the TCRs or antigen-binding fragments thereof provided herein are those that preferentially or selectively recognize or bind to an immunogenic (e.g., mismatched between donor and recipient of alloSCT) epitope or domain of HA-1, such as the immunogenic H peptide comprising the amino acid sequence VLHDDLLEA (SEQ ID NO:354), and do not recognize or bind to a non-immunogenic epitope or domain of HA-1, such as the non-immunogenic R peptide comprising the amino acid sequence VLRDDLLEA (SEQ ID NO:355), or exhibits a reduced affinity or selectivity for binding to the non-immunogenic epitope.

In some aspects, among the TCRs or antigen-binding fragments thereof provided herein are those that bind to or recognize an epitope of HA-1, such as the immunogenic H peptide comprising the amino acid sequence VLHDDLLEA (SEQ ID NO:354), that is complexed with an MHC molecule of a particular HLA type, such as HLA-A*02:01. In some aspects, among the TCRs or antigen-binding fragments thereof provided herein are those that bind to or recognize an epitope of HA-1, that is complexed with an MHC molecule of a particular HLA type, such as HLA-A*02:06 or B*40:01.

In some embodiments, a TCR provided herein is a full-length TCR. In some embodiments, a TCR provided herein is a dimeric TCR (dTCR). In some embodiments, TCR provided herein is a single-chain TCR (sc-TCR). A TCR provided herein may be cell-bound or in soluble form. In some embodiments, a TCR provided herein is in cell-bound form expressed on the surface of a cell (e.g., a T cell such as a T cell designed to lack expression of endogenous TCRs).

In some embodiments, a TCR provided herein is a scTCR, which is a single amino acid strand containing an a chain and a β chain that is able to bind to MHC-peptide complexes. Typically, a scTCR can be generated as described elsewhere, see, e.g., WO 96/13593, WO 96/18105, WO99/18129, WO 04/033685, WO2006/037960, WO2011/044186; U.S. Pat. No. 7,569,664; and Schlueter, C. J. et al. J. Mol. Biol. 256, 859 (1996).

### C. Exemplary Variable Domains

Provided herein are TCRs or antigen-binding fragments thereof that recognize or bind an epitope or region of a hematopoietically restricted minor histocompatibility antigen, such as HA-1, in the context of an MHC molecule. In some aspects, the HA-1 peptide is an immunogenic peptide (an allele of HA-1 that is mismatched between the donor and the recipient of an alloSCT). Provided are exemplary sequences (e.g. CDRs, Vα and/or VB, or Vγ and/or Vδ, and constant region sequences) of HA-1-specific TCRs.

In some embodiments, a TCR or antigen-binding fragment thereof provided herein binds to or recognizes an immunogenic HA-1 allele presented on the surface of leukemia cells of the recipient of an alloSCT. In some aspects, cytotoxic activity of T cells expressing the anti-HA-1 TCRs, is stimulated upon contact of the T cells with target cells presenting or expressing the antigen, such as an immunogenic HA-1 peptide. In some embodiments, among the provided TCRs or antigen-binding fragments thereof provided herein are those that bind or recognize a peptide epitope of HA-1 (e.g. a peptide epitope of an immunogenic allele of HA-1) in the context of an MHC, such as a particular MHC or a particular HLA subtype.

Among such TCRs or antigen-binding fragments thereof are TCRs or antigen-binding fragments thereof that contain any of the Vα region and Vβ region, or Vγ region and Vδ region, sequences as described, individually, or a sufficient antigen-binding portion of such sequences. In some embodiments, the provided TCRs or antigen-binding fragments thereof (e.g. anti-HA-1 TCRs) contain a Vα or Vγ region sequence or sufficient antigen-binding portion thereof that contains a CDR-1, a CDR-2 and/or a CDR-3 as described herein. In some embodiments, the provided TCRs or antigen-binding fragments thereof (e.g., anti-HA-1 TCRs) contain a Vβ or Vδ region sequence or sufficient antigen-binding portion thereof that contains a CDR-1, a CDR-2 and/or a CDR-3 as described herein. In some embodiments, the TCRs or antigen-binding fragments thereof (e.g. anti-HA-1 TCRs) contain a Vα or Vγ region sequence that contains a CDR-1, a CDR-2 and/or a CDR-3 as described herein and contain a Vβ or Vδ region sequence that contains a CDR-1, a CDR-2 and/or a CDR-3 as described herein. Also among the provided TCRs are those having sequences at least at or about 90%, 91%, 92%, 93%, 94%, 95%, 96%, 97%, 98%, or 99% identical to such a sequence.

In some embodiments, a TCR or antigen-binding fragment thereof provided herein contains a Vα or Vγ region containing a CDR-3 comprising an amino acid sequence set forth in any of SEQ ID NOs: 3, 21, 37, 51, 65, 78, 92, 106, 120, 136, 152, 166, 180, 194, 208, 224, 238, 252, 268, 278, 359, 369, 379, 389, 399, 409, 419, 429, 439, 449, 459, 460, 461, 462, 463, 464, 470, 471, 472, 473, 474, 476, 477, and 480, or a sequence having at least at or about 90%, 91%, 92%, 93%, 94%, 95%, 96%, 97%, 98%, or 99% identity with such a sequence. In some aspects, a TCR or antigen-binding fragment thereof provided herein contains a Vα or Vγ region containing a CDR-3 contained within the amino acid sequence set forth in any of SEQ ID NOs: 4, 22, 38, 52, 66, 79, 93, 107, 121, 137, 153, 167, 181, 195, 209, 225, 239, 253, 269, 279, 360, 370, 380, 390, 400, 410, 420, 430, 440, 450, and 481 or a sequence at least at or about 90%, 91%, 92%, 93%, 94%, 95%, 96%, 97%, 98%, or 99% identical with such a sequence.

In some embodiments, the Vα or Vγ region contains a CDR-1 comprising an amino acid sequence set forth in any of SEQ ID NOs: 1, 19, 35, 76, 134, 150, 222, and 266, or a sequence having at least at or about 90%, 91%, 92%, 93%, 94%, 95%, 96%, 97%, 98%, or 99% identity with such a sequence. In some aspects, the Vα or Vγ region contains a CDR-1 contained within the amino acid sequence set forth in any of SEQ ID NOs: 4, 22, 38, 52, 66, 79, 93, 107, 121, 137, 153, 167, 181, 195, 209, 225, 239, 253, 269, 279, 360, 370, 380, 390, 400, 410, 420, 430, 440, 450, and 481, or a sequence having at least at or about 90%, 91%, 92%, 93%, 94%, 95%, 96%, 97%, 98%, or 99% identity with such a sequence. In some embodiments, the Vα or Vγ region contains a CDR-2 comprising an amino acid sequence set forth in any of SEQ ID NOs: 2, 20, 36, 77, 135, 151, 223, and 267, or a sequence having at least at or about 90%, 91%, 92%, 93%, 94%, 95%, 96%, 97%, 98%, or 99% identity with such a sequence. In some embodiments, the Vα or Vγ region contains a CDR-2 contained within the amino acid sequence set forth in any of SEQ ID NOs: 4, 22, 38, 52, 66, 79, 93, 107, 121, 137, 153, 167, 181, 195, 209, 225, 239, 253, 269, 279, 360, 370, 380, 390, 400, 410, 420, 430, 440, 450, and 481, or a sequence having at least at or about 90%, 91%, 92%, 93%, 94%, 95%, 96%, 97%, 98%, or 99% identity with such a sequence.

In some instances, a TCR or antigen-binding fragment thereof provided herein contains a Vβ or Vδ region containing a CDR-3 comprising an amino acid sequence set forth in any of SEQ ID NOs: 11, 27, 43, 57, 84, 98, 112, 126, 142, 158, 172, 186, 200, 214, 230, 244, 258, 284, 363, 373, 383, 393, 403, 413, 423, 433, 443, 453, 465, 466, 467, 468, 469, 475, 478, 479, and 484 or a sequence having at least at or about 90%, 91%, 92%, 93%, 94%, 95%, 96%, 97%, 98%, or 99% identity with such a sequence. In some embodiments, a TCR or antigen-binding fragment thereof provided herein contains a Vβ or Vδ region containing a CDR-3 contained within the amino acid sequence set forth in any of SEQ ID NOs: 12, 28, 44, 58, 85, 99, 113, 127, 143, 159, 173, 187, 201, 215, 231, 245, 259, 285, 364, 374, 384, 394, 404, 414, 424, 434, 444, 454, and 485, or a sequence at least at or about 90%, 91%, 92%, 93%, 94%, 95%, 96%, 97%, 98%, or 99% identical with such a sequence.

In some instances, the Vβ or Vδ region contains a CDR-1 comprising an amino acid sequence set forth in SEQ ID NO: 9, or a sequence having at least at or about 90%, 91%, 92%, 93%, 94%, 95%, 96%, 97%, 98%, or 99% identity with such a sequence. In some aspects, the Vβ or Vδ region contains a CDR-1 contained within the amino acid sequence set forth in any of SEQ ID NOs: 12, 28, 44, 58, 85, 99, 113, 127, 143, 159, 173, 187, 201, 215, 231, 245, 259, 285, 364, 374, 384, 394, 404, 414, 424, 434, 444, 454, and 485, or a sequence having at least at or about 90%, 91%, 92%, 93%, 94%, 95%, 96%, 97%, 98%, or 99% identity with such a sequence. In some embodiments, the Vβ or Vδ region contains a CDR-2 comprising an amino acid sequence set forth in SEQ ID NO: 10, or a sequence having at least at or about 90%, 91%, 92%, 93%, 94%, 95%, 96%, 97%, 98%, or 99% identity with such a sequence. In some embodiments, the Vβ or Vδ region contains a CDR-2 contained within the amino acid sequence set forth in any of SEQ ID NOs: 12, 28, 44, 58, 85, 99, 113, 127, 143, 159, 173, 187, 201, 215, 231, 245, 259, 285, 364, 374, 384, 394, 404, 414, 424, 434, 444, 454, and 485, or a sequence having at least at or about 90%, 91%, 92%, 93%, 94%, 95%, 96%, 97%, 98%, or 99% identity with such a sequence.

In some embodiments, the Vα or Vγ region contains the amino acid sequence set forth in any of SEQ ID NOs: 4, 22, 38, 52, 66, 79, 93, 107, 121, 137, 153, 167, 181, 195, 209, 225, 239, 253, 269, 279, 360, 370, 380, 390, 400, 410, 420, 430, 440, 450, and 481, or a sequence that has at least 90%, 91%, 92%, 93%, 94%, 95%, 96%, 97%, 98%, or 99% sequence identity thereto. In some instances, the Vβ or Vδ region contains the amino acid sequence set forth in any of SEQ ID NOs: 12, 28, 44, 58, 85, 99, 113, 127, 143, 159, 173, 187, 201, 215, 231, 245, 259, 285, 364, 374, 384, 394, 404, 414, 424, 434, 444, 454, and 485, or a sequence that has at least 90%, 91%, 92%, 93%, 94%, 95%, 96%, 97%, 98%, or 99% sequence identity thereto. In some embodiments, the TCR contains an alpha chain comprising any of such Vα or Vγ region sequences and any of such Vβ or Vδ region sequences.

In one embodiment, a TCR or antigen binding fragment thereof provided herein having the ability to bind to a peptide epitope of a minor histocompatibility antigen HA-1 in the context of an MHC molecule can include an alpha chain (or gamma chain) having a CDR-1 having the amino acid sequence set forth in SEQ ID NO:1 (or a variant of SEQ ID NO:1 with one or two amino acid modifications), a CDR-2 having the amino acid sequence set forth in SEQ ID NO:2 (or a variant of SEQ ID NO:2 with one or two amino acid modifications), and a CDR-3 having the amino acid sequence set forth in SEQ ID NO:3 (or a variant of SEQ ID NO:3 with one or two amino acid modifications) and a beta chain (or delta chain) having a CDR-1 having the amino acid sequence set forth in SEQ ID NO:9 (or a variant of SEQ ID NO:9 with one or two amino acid modifications), a CDR-2 having the amino acid sequence set forth in SEQ ID NO: 10 (or a variant of SEQ ID NO:10 with one or two amino acid modifications), and a CDR-3 having the amino acid sequence set forth in SEQ ID NO:11 (or a variant of SEQ ID NO:11 with one or two amino acid modifications). An example of such a TCR having these CDRs and the ability to bind to a peptide epitope of a minor histocompatibility antigen HA-1 in the context of an MHC molecule includes, without limitation, TCR A.

In some cases, a TCR or antigen binding fragment thereof provided herein having the ability to bind to a peptide epitope of a minor histocompatibility antigen HA-1 in the context of an MHC molecule and having an alpha chain (or gamma chain) having a CDR-1 having the amino acid sequence set forth in SEQ ID NO:1 (or a variant of SEQ ID NO:1 with one or two amino acid modifications), a CDR-2 having the amino acid sequence set forth in SEQ ID NO:2 (or a variant of SEQ ID NO:2 with one or two amino acid modifications), and a CDR-3 having the amino acid sequence set forth in SEQ ID NO:3 (or a variant of SEQ ID NO:3 with one or two amino acid modifications) and a beta chain (or delta chain) having a CDR-1 having the amino acid sequence set forth in SEQ ID NO:9 (or a variant of SEQ ID NO:9 with one or two amino acid modifications), a CDR-2 having the amino acid sequence set forth in SEQ ID NO:10 (or a variant of SEQ ID NO:10 with one or two amino acid modifications), and a CDR-3 having the amino acid sequence set forth in SEQ ID NO: 11 (or a variant of SEQ ID NO: 11 with one or two amino acid modifications) can include any appropriate framework regions. For example, such a TCR or antigen binding fragment thereof can include an alpha chain that includes a framework region 1 having the entire amino acid sequence set forth in SEQ ID NO:4 that is upstream of the amino acid sequence of SEQ ID NO: 1 (or a variant of that sequence with one, two, three, four, five, six, seven, eight, nine, ten, or more amino acid modifications), a framework region 2 having the entire amino acid sequence set forth in SEQ ID NO:4 that is between the amino acid sequences of SEQ ID NOs: 1 and 2 (or a variant of that sequence with one, two, three, four, five, six, seven, eight, nine, ten, or more amino acid modifications), a framework region 3 having the entire amino acid sequence set forth in SEQ ID NO:4 that is between the amino acid sequences of SEQ ID NOs:2 and 3 (or a variant of that sequence with one, two, three, four, five, six, seven, eight, nine, ten, or more amino acid modifications), and a framework region 4 having the entire amino acid sequence set forth in SEQ ID NO:4 that is downstream of the amino acid sequence of SEQ ID NO:3 (or a variant of that sequence with one, two, three, four, five, six, seven, eight, nine, ten, or more amino acid modifications) and a beta chain that includes a framework region 1 having the entire amino acid sequence set forth in SEQ ID NO: 12 that is upstream of the amino acid sequence of SEQ ID NO:9 (or a variant of that sequence with one, two, three, four, five, six, seven, eight, nine, ten, or more amino acid modifications), a framework region 2 having the entire amino acid sequence set forth in SEQ ID NO: 12 that is between the amino acid sequences of SEQ ID NOs:9 and 10 (or a variant of that sequence with one, two, three, four, five, six, seven, eight, nine, ten, or more amino acid modifications), a framework region 3 having the entire amino acid sequence set forth in SEQ ID NO:12 that is between the amino acid sequences of SEQ ID NOs:10 and 11 (or a variant of that sequence with one, two, three, four, five, six, seven, eight, nine, ten, or more amino acid modifications), and a framework region 4 having the entire amino acid sequence set forth in SEQ ID NO: 12 that is downstream of the amino acid sequence of SEQ ID NO: 11 (or a variant of that sequence with one, two, three, four, five, six, seven, eight, nine, ten, or more amino acid modifications).

In some cases, a TCR or antigen binding fragment thereof provided herein having the ability to bind to a peptide epitope of a minor histocompatibility antigen HA-1 in the context of an MHC molecule can include an alpha chain that includes an amino acid sequence having at least 90 percent identity to the amino acid sequence set forth in SEQ ID NO:4 and a beta chain that includes an amino acid sequence having at least 90 percent identity to the amino acid sequence set forth in SEQ ID NO: 12. For example, a TCR or antigen binding fragment thereof provided herein can include an alpha chain that includes an amino acid sequence having at least 90, 91, 92, 93, 94, 95, 96, 97, 98, or 99 percent identity to the amino acid sequence set forth in SEQ ID NO:4 and a beta chain that includes an amino acid sequence having at least 90, 91, 92, 93, 94, 95, 96, 97, 98, or 99 percent identity to the amino acid sequence set forth in SEQ ID NO:12. In some cases, a TCR or antigen binding fragment thereof provided herein can include (a) an alpha chain that includes an amino acid sequence having 100 percent identity to the amino acid sequence set forth in SEQ ID NO:4, and (b) a beta chain that includes an amino acid sequence having 100 percent identity to the amino acid sequence set forth in SEQ ID NO:12.

In some cases, a TCR or antigen binding fragment thereof provided herein having the ability to bind to a peptide epitope of a minor histocompatibility antigen HA-1 in the context of an MHC molecule can include (a) an alpha chain that includes an amino acid sequence having at least 90 percent identity to the amino acid sequence set forth in SEQ ID NO:4, provided that the alpha chain includes the amino acid sequences set forth in SEQ ID NOs: 1, 2, and 3 and (b) a beta chain that includes an amino acid sequence having at least 90 percent identity to the amino acid sequence set forth in SEQ ID NO: 12, provided that the beta chain includes the amino acid sequences set forth in SEQ ID NOs:9, 10, and 11. For example, a TCR or antigen binding fragment thereof provided herein can include (a) an alpha chain that includes an amino acid sequence having at least 90, 91, 92, 93, 94, 95, 96, 97, 98, or 99 percent identity to the amino acid sequence set forth in SEQ ID NO:4, provided that the alpha chain includes the amino acid sequences set forth in SEQ ID NOs: 1, 2, and 3 and (b) a beta chain that includes an amino acid sequence having at least 90, 91, 92, 93, 94, 95, 96, 97, 98, or 99 percent identity to the amino acid sequence set forth in SEQ ID NO:12, provided that the beta chain includes the amino acid sequences set forth in SEQ ID NOs:9, 10, and 11.

In some cases, a TCR or antigen binding fragment thereof provided herein having the ability to bind to a peptide epitope of a minor histocompatibility antigen HA-1 in the context of an MHC molecule can include (a) an alpha chain having the amino acid sequence set forth in SEQ ID NO:4 or the amino acid set forth in SEQ ID NO:4 with one, two, three, four, five, six, seven, eight, nine, or 10 amino acid modifications (e.g., amino acid substitutions, amino acid deletions, and/or amino acid additions) and (b) a beta chain having the amino acid sequence set forth in SEQ ID NO: 12 or the amino acid set forth in SEQ ID NO: 12 with one, two, three, four, five, six, seven, eight, nine, or 10 amino acid modifications (e.g., amino acid substitutions, amino acid deletions, and/or amino acid additions). For example, a TCR or antigen binding fragment thereof provided herein (a) can have the ability to bind to a peptide epitope of a minor histocompatibility antigen HA-1 in the context of an MHC molecule, (b) can include an alpha chain having the amino acid sequence set forth in SEQ ID NO:4 with one, two, three, four, five, six, seven, eight, nine, or 10 amino acid modifications (e.g., amino acid substitutions, amino acid deletions, and/or amino acid additions), provided that the alpha chain includes the amino acid sequences set forth in SEQ ID NOs: 1, 2, and 3, and (c) can include a beta chain having the amino acid sequence set forth in SEQ ID NO: 12 with one, two, three, four, five, six, seven, eight, nine, or 10 amino acid modifications (e.g., amino acid substitutions, amino acid deletions, and/or amino acid additions), provided that the beta chain includes the amino acid sequences set forth in SEQ ID NOs:9, 10, and 11.

In one embodiment, a TCR or antigen binding fragment thereof provided herein having the ability to bind to a peptide epitope of a minor histocompatibility antigen HA-1 in the context of an MHC molecule can include an alpha chain (or gamma chain) having a CDR-1 having the amino acid sequence set forth in SEQ ID NO: 19 (or a variant of SEQ ID NO: 19 with one or two amino acid modifications), a CDR-2 having the amino acid sequence set forth in SEQ ID NO:20 (or a variant of SEQ ID NO:20 with one or two amino acid modifications), and a CDR-3 having the amino acid sequence set forth in SEQ ID NO:21 (or a variant of SEQ ID NO:21 with one or two amino acid modifications) and a beta chain (or delta chain) having a CDR-1 having the amino acid sequence set forth in SEQ ID NO:9 (or a variant of SEQ ID NO:9 with one or two amino acid modifications), a CDR-2 having the amino acid sequence set forth in SEQ ID NO: 10 (or a variant of SEQ ID NO:10 with one or two amino acid modifications), and a CDR-3 having the amino acid sequence set forth in SEQ ID NO:27 (or a variant of SEQ ID NO:27 with one or two amino acid modifications). An example of such a TCR having these CDRs and the ability to bind to a peptide epitope of a minor histocompatibility antigen HA-1 in the context of an MHC molecule includes, without limitation, TCR B.

In some cases, a TCR or antigen binding fragment thereof provided herein having the ability to bind to a peptide epitope of a minor histocompatibility antigen HA-1 in the context of an MHC molecule and having an alpha chain (or gamma chain) having a CDR-1 having the amino acid sequence set forth in SEQ ID NO:19 (or a variant of SEQ ID NO:19 with one or two amino acid modifications), a CDR-2 having the amino acid sequence set forth in SEQ ID NO:20 (or a variant of SEQ ID NO:20 with one or two amino acid modifications), and a CDR-3 having the amino acid sequence set forth in SEQ ID NO:21 (or a variant of SEQ ID NO:21 with one or two amino acid modifications) and a beta chain (or delta chain) having a CDR-1 having the amino acid sequence set forth in SEQ ID NO:9 (or a variant of SEQ ID NO:9 with one or two amino acid modifications), a CDR-2 having the amino acid sequence set forth in SEQ ID NO:10 (or a variant of SEQ ID NO:10 with one or two amino acid modifications), and a CDR-3 having the amino acid sequence set forth in SEQ ID NO:27 (or a variant of SEQ ID NO:27 with one or two amino acid modifications) can include any appropriate framework regions. For example, such a TCR or antigen binding fragment thereof can include an alpha chain that includes a framework region 1 having the entire amino acid sequence set forth in SEQ ID NO:22 that is upstream of the amino acid sequence of SEQ ID NO: 19 (or a variant of that sequence with one, two, three, four, five, six, seven, eight, nine, ten, or more amino acid modifications), a framework region 2 having the entire amino acid sequence set forth in SEQ ID NO:22 that is between the amino acid sequences of SEQ ID NOs:19 and 20 (or a variant of that sequence with one, two, three, four, five, six, seven, eight, nine, ten, or more amino acid modifications), a framework region 3 having the entire amino acid sequence set forth in SEQ ID NO:22 that is between the amino acid sequences of SEQ ID NOs:20 and 21 (or a variant of that sequence with one, two, three, four, five, six, seven, eight, nine, ten, or more amino acid modifications), and a framework region 4 having the entire amino acid sequence set forth in SEQ ID NO:22 that is downstream of the amino acid sequence of SEQ ID NO:21 (or a variant of that sequence with one, two, three, four, five, six, seven, eight, nine, ten, or more amino acid modifications) and a beta chain that includes a framework region 1 having the entire amino acid sequence set forth in SEQ ID NO:28 that is upstream of the amino acid sequence of SEQ ID NO:9 (or a variant of that sequence with one, two, three, four, five, six, seven, eight, nine, ten, or more amino acid modifications), a framework region 2 having the entire amino acid sequence set forth in SEQ ID NO:28 that is between the amino acid sequences of SEQ ID NOs:9 and 10 (or a variant of that sequence with one, two, three, four, five, six, seven, eight, nine, ten, or more amino acid modifications), a framework region 3 having the entire amino acid sequence set forth in SEQ ID NO:28 that is between the amino acid sequences of SEQ ID NOs: 10 and 27 (or a variant of that sequence with one, two, three, four, five, six, seven, eight, nine, ten, or more amino acid modifications), and a framework region 4 having the entire amino acid sequence set forth in SEQ ID NO:28 that is downstream of the amino acid sequence of SEQ ID NO:27 (or a variant of that sequence with one, two, three, four, five, six, seven, eight, nine, ten, or more amino acid modifications).

In some cases, a TCR or antigen binding fragment thereof provided herein having the ability to bind to a peptide epitope of a minor histocompatibility antigen HA-1 in the context of an MHC molecule can include an alpha chain that includes an amino acid sequence having at least 90 percent identity to the amino acid sequence set forth in SEQ ID NO:22 and a beta chain that includes an amino acid sequence having at least 90 percent identity to the amino acid sequence set forth in SEQ ID NO:28. For example, a TCR or antigen binding fragment thereof provided herein can include an alpha chain that includes an amino acid sequence having at least 90, 91, 92, 93, 94, 95, 96, 97, 98, or 99 percent identity to the amino acid sequence set forth in SEQ ID NO:22 and a beta chain that includes an amino acid sequence having at least 90, 91, 92, 93, 94, 95, 96, 97, 98, or 99 percent identity to the amino acid sequence set forth in SEQ ID NO:28. In some cases, a TCR or antigen binding fragment thereof provided herein can include (a) an alpha chain that includes an amino acid sequence having 100 percent identity to the amino acid sequence set forth in SEQ ID NO:22, and (b) a beta chain that includes an amino acid sequence having 100 percent identity to the amino acid sequence set forth in SEQ ID NO:28.

In some cases, a TCR or antigen binding fragment thereof provided herein having the ability to bind to a peptide epitope of a minor histocompatibility antigen HA-1 in the context of an MHC molecule can include (a) an alpha chain that includes an amino acid sequence having at least 90 percent identity to the amino acid sequence set forth in SEQ ID NO:22, provided that the alpha chain includes the amino acid sequences set forth in SEQ ID NOs: 19, 20, and 21 and (b) a beta chain that includes an amino acid sequence having at least 90 percent identity to the amino acid sequence set forth in SEQ ID NO:28, provided that the beta chain includes the amino acid sequences set forth in SEQ ID NOs:9, 10, and 27. For example, a TCR or antigen binding fragment thereof provided herein can include (a) an alpha chain that includes an amino acid sequence having at least 90, 91, 92, 93, 94, 95, 96, 97, 98, or 99 percent identity to the amino acid sequence set forth in SEQ ID NO:22, provided that the alpha chain includes the amino acid sequences set forth in SEQ ID NOs: 19, 20, and 21 and (b) a beta chain that includes an amino acid sequence having at least 90, 91, 92, 93, 94, 95, 96, 97, 98, or 99 percent identity to the amino acid sequence set forth in SEQ ID NO:28, provided that the beta chain includes the amino acid sequences set forth in SEQ ID NOs:9, 10, and 27.

In some cases, a TCR or antigen binding fragment thereof provided herein having the ability to bind to a peptide epitope of a minor histocompatibility antigen HA-1 in the context of an MHC molecule can include (a) an alpha chain having the amino acid sequence set forth in SEQ ID NO:22 or the amino acid set forth in SEQ ID NO:22 with one, two, three, four, five, six, seven, eight, nine, or 10 amino acid modifications (e.g., amino acid substitutions, amino acid deletions, and/or amino acid additions) and (b) a beta chain having the amino acid sequence set forth in SEQ ID NO:28 or the amino acid set forth in SEQ ID NO:28 with one, two, three, four, five, six, seven, eight, nine, or 10 amino acid modifications (e.g., amino acid substitutions, amino acid deletions, and/or amino acid additions). For example, a TCR or antigen binding fragment thereof provided herein (a) can have the ability to bind to a peptide epitope of a minor histocompatibility antigen HA-1 in the context of an MHC molecule, (b) can include an alpha chain having the amino acid sequence set forth in SEQ ID NO:22 with one, two, three, four, five, six, seven, eight, nine, or 10 amino acid modifications (e.g., amino acid substitutions, amino acid deletions, and/or amino acid additions), provided that the alpha chain includes the amino acid sequences set forth in SEQ ID NOs: 19, 20, and 21, and (c) can include a beta chain having the amino acid sequence set forth in SEQ ID NO:28 with one, two, three, four, five, six, seven, eight, nine, or 10 amino acid modifications (e.g., amino acid substitutions, amino acid deletions, and/or amino acid additions), provided that the beta chain includes the amino acid sequences set forth in SEQ ID NOs:9, 10, and 27.

In one embodiment, a TCR or antigen binding fragment thereof provided herein having the ability to bind to a peptide epitope of a minor histocompatibility antigen HA-1 in the context of an MHC molecule can include an alpha chain (or gamma chain) having a CDR-1 having the amino acid sequence set forth in SEQ ID NO:35 (or a variant of SEQ ID NO:35 with one or two amino acid modifications), a CDR-2 having the amino acid sequence set forth in SEQ ID NO:36 (or a variant of SEQ ID NO:36 with one or two amino acid modifications), and a CDR-3 having the amino acid sequence set forth in SEQ ID NO:37 (or a variant of SEQ ID NO:37 with one or two amino acid modifications) and a beta chain (or delta chain) having a CDR-1 having the amino acid sequence set forth in SEQ ID NO:9 (or a variant of SEQ ID NO:9 with one or two amino acid modifications), a CDR-2 having the amino acid sequence set forth in SEQ ID NO: 10 (or a variant of SEQ ID NO:10 with one or two amino acid modifications), and a CDR-3 having the amino acid sequence set forth in SEQ ID NO:43 (or a variant of SEQ ID NO:43 with one or two amino acid modifications). An example of such a TCR having these CDRs and the ability to bind to a peptide epitope of a minor histocompatibility antigen HA-1 in the context of an MHC molecule includes, without limitation, TCR C.

In some cases, a TCR or antigen binding fragment thereof provided herein having the ability to bind to a peptide epitope of a minor histocompatibility antigen HA-1 in the context of an MHC molecule and having an alpha chain (or gamma chain) having a CDR-1 having the amino acid sequence set forth in SEQ ID NO:35 (or a variant of SEQ ID NO:35 with one or two amino acid modifications), a CDR-2 having the amino acid sequence set forth in SEQ ID NO:36 (or a variant of SEQ ID NO:36 with one or two amino acid modifications), and a CDR-3 having the amino acid sequence set forth in SEQ ID NO:37 (or a variant of SEQ ID NO:37 with one or two amino acid modifications) and a beta chain (or delta chain) having a CDR-1 having the amino acid sequence set forth in SEQ ID NO:9 (or a variant of SEQ ID NO:9 with one or two amino acid modifications), a CDR-2 having the amino acid sequence set forth in SEQ ID NO:10 (or a variant of SEQ ID NO:10 with one or two amino acid modifications), and a CDR-3 having the amino acid sequence set forth in SEQ ID NO:43 (or a variant of SEQ ID NO:43 with one or two amino acid modifications) can include any appropriate framework regions. For example, such a TCR or antigen binding fragment thereof can include an alpha chain that includes a framework region 1 having the entire amino acid sequence set forth in SEQ ID NO:38 that is upstream of the amino acid sequence of SEQ ID NO:35 (or a variant of that sequence with one, two, three, four, five, six, seven, eight, nine, ten, or more amino acid modifications), a framework region 2 having the entire amino acid sequence set forth in SEQ ID NO:38 that is between the amino acid sequences of SEQ ID NOs:35 and 36 (or a variant of that sequence with one, two, three, four, five, six, seven, eight, nine, ten, or more amino acid modifications), a framework region 3 having the entire amino acid sequence set forth in SEQ ID NO:38 that is between the amino acid sequences of SEQ ID NOs:36 and 37 (or a variant of that sequence with one, two, three, four, five, six, seven, eight, nine, ten, or more amino acid modifications), and a framework region 4 having the entire amino acid sequence set forth in SEQ ID NO:38 that is downstream of the amino acid sequence of SEQ ID NO:37 (or a variant of that sequence with one, two, three, four, five, six, seven, eight, nine, ten, or more amino acid modifications) and a beta chain that includes a framework region 1 having the entire amino acid sequence set forth in SEQ ID NO:44 that is upstream of the amino acid sequence of SEQ ID NO:9 (or a variant of that sequence with one, two, three, four, five, six, seven, eight, nine, ten, or more amino acid modifications), a framework region 2 having the entire amino acid sequence set forth in SEQ ID NO:44 that is between the amino acid sequences of SEQ ID NOs:9 and 10 (or a variant of that sequence with one, two, three, four, five, six, seven, eight, nine, ten, or more amino acid modifications), a framework region 3 having the entire amino acid sequence set forth in SEQ ID NO:44 that is between the amino acid sequences of SEQ ID NOs: 10 and 43 (or a variant of that sequence with one, two, three, four, five, six, seven, eight, nine, ten, or more amino acid modifications), and a framework region 4 having the entire amino acid sequence set forth in SEQ ID NO:44 that is downstream of the amino acid sequence of SEQ ID NO:43 (or a variant of that sequence with one, two, three, four, five, six, seven, eight, nine, ten, or more amino acid modifications).

In some cases, a TCR or antigen binding fragment thereof provided herein having the ability to bind to a peptide epitope of a minor histocompatibility antigen HA-1 in the context of an MHC molecule can include an alpha chain that includes an amino acid sequence having at least 90 percent identity to the amino acid sequence set forth in SEQ ID NO:38 and a beta chain that includes an amino acid sequence having at least 90 percent identity to the amino acid sequence set forth in SEQ ID NO:44. For example, a TCR or antigen binding fragment thereof provided herein can include an alpha chain that includes an amino acid sequence having at least 90, 91, 92, 93, 94, 95, 96, 97, 98, or 99 percent identity to the amino acid sequence set forth in SEQ ID NO:38 and a beta chain that includes an amino acid sequence having at least 90, 91, 92, 93, 94, 95, 96, 97, 98, or 99 percent identity to the amino acid sequence set forth in SEQ ID NO:44. In some cases, a TCR or antigen binding fragment thereof provided herein can include (a) an alpha chain that includes an amino acid sequence having 100 percent identity to the amino acid sequence set forth in SEQ ID NO:38, and (b) a beta chain that includes an amino acid sequence having 100 percent identity to the amino acid sequence set forth in SEQ ID NO:44.

In some cases, a TCR or antigen binding fragment thereof provided herein having the ability to bind to a peptide epitope of a minor histocompatibility antigen HA-1 in the context of an MHC molecule can include (a) an alpha chain that includes an amino acid sequence having at least 90 percent identity to the amino acid sequence set forth in SEQ ID NO:38, provided that the alpha chain includes the amino acid sequences set forth in SEQ ID NOs:35, 36, and 37 and (b) a beta chain that includes an amino acid sequence having at least 90 percent identity to the amino acid sequence set forth in SEQ ID NO:44, provided that the beta chain includes the amino acid sequences set forth in SEQ ID NOs:9, 10, and 43. For example, a TCR or antigen binding fragment thereof provided herein can include (a) an alpha chain that includes an amino acid sequence having at least 90, 91, 92, 93, 94, 95, 96, 97, 98, or 99 percent identity to the amino acid sequence set forth in SEQ ID NO:38, provided that the alpha chain includes the amino acid sequences set forth in SEQ ID NOs: 35, 36, and 37 and (b) a beta chain that includes an amino acid sequence having at least 90, 91, 92, 93, 94, 95, 96, 97, 98, or 99 percent identity to the amino acid sequence set forth in SEQ ID NO:44, provided that the beta chain includes the amino acid sequences set forth in SEQ ID NOs:9, 10, and 43.

In some cases, a TCR or antigen binding fragment thereof provided herein having the ability to bind to a peptide epitope of a minor histocompatibility antigen HA-1 in the context of an MHC molecule can include (a) an alpha chain having the amino acid sequence set forth in SEQ ID NO:38 or the amino acid set forth in SEQ ID NO:38 with one, two, three, four, five, six, seven, eight, nine, or 10 amino acid modifications (e.g., amino acid substitutions, amino acid deletions, and/or amino acid additions) and (b) a beta chain having the amino acid sequence set forth in SEQ ID NO:44 or the amino acid set forth in SEQ ID NO:44 with one, two, three, four, five, six, seven, eight, nine, or 10 amino acid modifications (e.g., amino acid substitutions, amino acid deletions, and/or amino acid additions). For example, a TCR or antigen binding fragment thereof provided herein (a) can have the ability to bind to a peptide epitope of a minor histocompatibility antigen HA-1 in the context of an MHC molecule, (b) can include an alpha chain having the amino acid sequence set forth in SEQ ID NO:38 with one, two, three, four, five, six, seven, eight, nine, or 10 amino acid modifications (e.g., amino acid substitutions, amino acid deletions, and/or amino acid additions), provided that the alpha chain includes the amino acid sequences set forth in SEQ ID NOs: 35, 36, and 37, and (c) can include a beta chain having the amino acid sequence set forth in SEQ ID NO:44 with one, two, three, four, five, six, seven, eight, nine, or 10 amino acid modifications (e.g., amino acid substitutions, amino acid deletions, and/or amino acid additions), provided that the beta chain includes the amino acid sequences set forth in SEQ ID NOs:9, 10, and 43.

In one embodiment, a TCR or antigen binding fragment thereof provided herein having the ability to bind to a peptide epitope of a minor histocompatibility antigen HA-1 in the context of an MHC molecule can include an alpha chain (or gamma chain) having a CDR-1 having the amino acid sequence set forth in SEQ ID NO: 1 (or a variant of SEQ ID NO: 1 with one or two amino acid modifications), a CDR-2 having the amino acid sequence set forth in SEQ ID NO:2 (or a variant of SEQ ID NO:2 with one or two amino acid modifications), and a CDR-3 having the amino acid sequence set forth in SEQ ID NO:51 (or a variant of SEQ ID NO:51 with one or two amino acid modifications) and a beta chain (or delta chain) having a CDR-1 having the amino acid sequence set forth in SEQ ID NO:9 (or a variant of SEQ ID NO:9 with one or two amino acid modifications), a CDR-2 having the amino acid sequence set forth in SEQ ID NO: 10 (or a variant of SEQ ID NO:10 with one or two amino acid modifications), and a CDR-3 having the amino acid sequence set forth in SEQ ID NO:57 (or a variant of SEQ ID NO:57 with one or two amino acid modifications). An example of such a TCR having these CDRs and the ability to bind to a peptide epitope of a minor histocompatibility antigen HA-1 in the context of an MHC molecule includes, without limitation, TCR D.

In some cases, a TCR or antigen binding fragment thereof provided herein having the ability to bind to a peptide epitope of a minor histocompatibility antigen HA-1 in the context of an MHC molecule and having an alpha chain (or gamma chain) having a CDR-1 having the amino acid sequence set forth in SEQ ID NO:1 (or a variant of SEQ ID NO:1 with one or two amino acid modifications), a CDR-2 having the amino acid sequence set forth in SEQ ID NO:2 (or a variant of SEQ ID NO:2 with one or two amino acid modifications), and a CDR-3 having the amino acid sequence set forth in SEQ ID NO:51 (or a variant of SEQ ID NO:51 with one or two amino acid modifications) and a beta chain (or delta chain) having a CDR-1 having the amino acid sequence set forth in SEQ ID NO:9 (or a variant of SEQ ID NO:9 with one or two amino acid modifications), a CDR-2 having the amino acid sequence set forth in SEQ ID NO: 10 (or a variant of SEQ ID NO:10 with one or two amino acid modifications), and a CDR-3 having the amino acid sequence set forth in SEQ ID NO:57 (or a variant of SEQ ID NO:57 with one or two amino acid modifications) can include any appropriate framework regions. For example, such a TCR or antigen binding fragment thereof can include an alpha chain that includes a framework region 1 having the entire amino acid sequence set forth in SEQ ID NO:52 that is upstream of the amino acid sequence of SEQ ID NO: 1 (or a variant of that sequence with one, two, three, four, five, six, seven, eight, nine, ten, or more amino acid modifications), a framework region 2 having the entire amino acid sequence set forth in SEQ ID NO:52 that is between the amino acid sequences of SEQ ID NOs: 1 and 2 (or a variant of that sequence with one, two, three, four, five, six, seven, eight, nine, ten, or more amino acid modifications), a framework region 3 having the entire amino acid sequence set forth in SEQ ID NO:52 that is between the amino acid sequences of SEQ ID NOs:2 and 51 (or a variant of that sequence with one, two, three, four, five, six, seven, eight, nine, ten, or more amino acid modifications), and a framework region 4 having the entire amino acid sequence set forth in SEQ ID NO:52 that is downstream of the amino acid sequence of SEQ ID NO:51 (or a variant of that sequence with one, two, three, four, five, six, seven, eight, nine, ten, or more amino acid modifications) and a beta chain that includes a framework region 1 having the entire amino acid sequence set forth in SEQ ID NO:58 that is upstream of the amino acid sequence of SEQ ID NO:9 (or a variant of that sequence with one, two, three, four, five, six, seven, eight, nine, ten, or more amino acid modifications), a framework region 2 having the entire amino acid sequence set forth in SEQ ID NO:58 that is between the amino acid sequences of SEQ ID NOs:9 and 10 (or a variant of that sequence with one, two, three, four, five, six, seven, eight, nine, ten, or more amino acid modifications), a framework region 3 having the entire amino acid sequence set forth in SEQ ID NO:58 that is between the amino acid sequences of SEQ ID NOs: 10 and 57 (or a variant of that sequence with one, two, three, four, five, six, seven, eight, nine, ten, or more amino acid modifications), and a framework region 4 having the entire amino acid sequence set forth in SEQ ID NO:58 that is downstream of the amino acid sequence of SEQ ID NO:57 (or a variant of that sequence with one, two, three, four, five, six, seven, eight, nine, ten, or more amino acid modifications).

In some cases, a TCR or antigen binding fragment thereof provided herein having the ability to bind to a peptide epitope of a minor histocompatibility antigen HA-1 in the context of an MHC molecule can include an alpha chain that includes an amino acid sequence having at least 90 percent identity to the amino acid sequence set forth in SEQ ID NO:52 and a beta chain that includes an amino acid sequence having at least 90 percent identity to the amino acid sequence set forth in SEQ ID NO:58. For example, a TCR or antigen binding fragment thereof provided herein can include an alpha chain that includes an amino acid sequence having at least 90, 91, 92, 93, 94, 95, 96, 97, 98, or 99 percent identity to the amino acid sequence set forth in SEQ ID NO:52 and a beta chain that includes an amino acid sequence having at least 90, 91, 92, 93, 94, 95, 96, 97, 98, or 99 percent identity to the amino acid sequence set forth in SEQ ID NO:58. In some cases, a TCR or antigen binding fragment thereof provided herein can include (a) an alpha chain that includes an amino acid sequence having 100 percent identity to the amino acid sequence set forth in SEQ ID NO:52, and (b) a beta chain that includes an amino acid sequence having 100 percent identity to the amino acid sequence set forth in SEQ ID NO:58.

In some cases, a TCR or antigen binding fragment thereof provided herein having the ability to bind to a peptide epitope of a minor histocompatibility antigen HA-1 in the context of an MHC molecule can include (a) an alpha chain that includes an amino acid sequence having at least 90 percent identity to the amino acid sequence set forth in SEQ ID NO:52, provided that the alpha chain includes the amino acid sequences set forth in SEQ ID NOs:1, 2, and 51 and (b) a beta chain that includes an amino acid sequence having at least 90 percent identity to the amino acid sequence set forth in SEQ ID NO:58, provided that the beta chain includes the amino acid sequences set forth in SEQ ID NOs:9, 10, and 57. For example, a TCR or antigen binding fragment thereof provided herein can include (a) an alpha chain that includes an amino acid sequence having at least 90, 91, 92, 93, 94, 95, 96, 97, 98, or 99 percent identity to the amino acid sequence set forth in SEQ ID NO:52, provided that the alpha chain includes the amino acid sequences set forth in SEQ ID NOs: 1, 2, and 51 and (b) a beta chain that includes an amino acid sequence having at least 90, 91, 92, 93, 94, 95, 96, 97, 98, or 99 percent identity to the amino acid sequence set forth in SEQ ID NO:58, provided that the beta chain includes the amino acid sequences set forth in SEQ ID NOs:9, 10, and 57.

In some cases, a TCR or antigen binding fragment thereof provided herein having the ability to bind to a peptide epitope of a minor histocompatibility antigen HA-1 in the context of an MHC molecule can include (a) an alpha chain having the amino acid sequence set forth in SEQ ID NO:52 or the amino acid set forth in SEQ ID NO:52 with one, two, three, four, five, six, seven, eight, nine, or 10 amino acid modifications (e.g., amino acid substitutions, amino acid deletions, and/or amino acid additions) and (b) a beta chain having the amino acid sequence set forth in SEQ ID NO:58 or the amino acid set forth in SEQ ID NO:58 with one, two, three, four, five, six, seven, eight, nine, or 10 amino acid modifications (e.g., amino acid substitutions, amino acid deletions, and/or amino acid additions). For example, a TCR or antigen binding fragment thereof provided herein (a) can have the ability to bind to a peptide epitope of a minor histocompatibility antigen HA-1 in the context of an MHC molecule, (b) can include an alpha chain having the amino acid sequence set forth in SEQ ID NO:52 with one, two, three, four, five, six, seven, eight, nine, or 10 amino acid modifications (e.g., amino acid substitutions, amino acid deletions, and/or amino acid additions), provided that the alpha chain includes the amino acid sequences set forth in SEQ ID NOs: 1, 2, and 51, and (c) can include a beta chain having the amino acid sequence set forth in SEQ ID NO:58 with one, two, three, four, five, six, seven, eight, nine, or 10 amino acid modifications (e.g., amino acid substitutions, amino acid deletions, and/or amino acid additions), provided that the beta chain includes the amino acid sequences set forth in SEQ ID NOs:9, 10, and 57.

In some embodiments, a TCR or antigen-binding fragment thereof provided herein contains a Vα or Vγ region that contains a CDR-1, a CDR-2, and a CDR-3, comprising the sequences of SEQ ID NOs: 1, 2, 3, respectively; a CDR-1, a CDR-2, and a CDR-3, comprising SEQ ID NOs: 19, 20, 21, respectively; a CDR-1, a CDR-2, and a CDR-3, comprising SEQ ID NOs:35, 36, 37, respectively; a CDR-1, a CDR-2, and a CDR-3, comprising SEQ ID NOs:1, 2, 51, respectively; a CDR-1, a CDR-2, and a CDR-3, comprising SEQ ID NOs:1, 2, 65, respectively; a CDR-1, a CDR-2, and a CDR-3, comprising SEQ ID NOs:76, 77, 78, respectively; a CDR-1, a CDR-2, and a CDR-3, comprising SEQ ID NOs: 19, 20, 92, respectively; a CDR-1, a CDR-2, and a CDR-3, comprising SEQ ID NOs:76, 77, 106, respectively; a CDR-1, a CDR-2, and a CDR-3, comprising SEQ ID NOs:35, 36, 120, respectively; a CDR-1, a CDR-2, and a CDR-3, comprising SEQ ID NOs:134, 135, 136, respectively; a CDR-1, a CDR-2, and a CDR-3, comprising SEQ ID NOs: 150, 151, 152, respectively; a CDR-1, a CDR-2, and a CDR-3, comprising SEQ ID NOs:35, 36, 166, respectively; a CDR-1, a CDR-2, and a CDR-3, comprising SEQ ID NOs:35, 36, 180, respectively; a CDR-1, a CDR-2, and a CDR-3, comprising SEQ ID NOs:76, 77, 194, respectively; a CDR-1, a CDR-2, and a CDR-3, comprising SEQ ID NOs:76, 77, 208, respectively; a CDR-1, a CDR-2, and a CDR-3, comprising SEQ ID NOs:222, 223, 224, respectively; a CDR-1, a CDR-2, and a CDR-3, comprising SEQ ID NOs:76, 77, 238, respectively; a CDR-1, a CDR-2, and a CDR-3, comprising SEQ ID NOs: 19, 20, 252, respectively; a CDR-1, a CDR-2, and a CDR-3, comprising SEQ ID NOs:266, 267, 268, respectively; or a CDR-1, a CDR-2, and a CDR-3, comprising SEQ ID NOs: 19, 20, 278, respectively. In some embodiments, a TCR or antigen-binding fragment thereof provided herein contains a Vβ or Vδ region that contains a CDR-1, CDR-2, and CDR-3 comprising the sequences of SEQ ID NOs:9, 10, 11, respectively; a CDR-1, a CDR-2, and a CDR-3, comprising SEQ ID NOs:9, 10, 27, respectively; a CDR-1, a CDR-2, and a CDR-3, comprising SEQ ID NOs:9, 10, 43, respectively; a CDR-1, a CDR-2, and a CDR-3, comprising SEQ ID NOs:9, 10, 57, respectively; a CDR-1, a CDR-2, and a CDR-3, comprising SEQ ID NOs:9, 10, 57, respectively; a CDR-1, a CDR-2, and a CDR-3, comprising SEQ ID NOs:9, 10, 84, respectively; a CDR-1, a CDR-2, and a CDR-3, comprising SEQ ID NOs:9, 10, 98, respectively; a CDR-1, a CDR-2, and a CDR-3, comprising SEQ ID NOs:9, 10, 112, respectively; a CDR-1, a CDR-2, and a CDR-3, comprising SEQ ID NOs:9, 10, 126, respectively; a CDR-1, a CDR-2, and a CDR-3, comprising SEQ ID NOs:9, 10, 142, respectively; a CDR-1, a CDR-2, and a CDR-3, comprising SEQ ID NOs:9, 10, 158, respectively; a CDR-1, a CDR-2, and a CDR-3, comprising SEQ ID NOs:9, 10, 172, respectively; a CDR-1, a CDR-2, and a CDR-3, comprising SEQ ID NOs:9, 10, 186, respectively; a CDR-1, a CDR-2, and a CDR-3, comprising SEQ ID NOs:9, 10, 200, respectively; a CDR-1, a CDR-2, and a CDR-3, comprising SEQ ID NOs:9, 10, 214, respectively; a CDR-1, a CDR-2, and a CDR-3, comprising SEQ ID NOs:9, 10, 230, respectively; a CDR-1, a CDR-2, and a CDR-3, comprising SEQ ID NOs:9, 10, 244, respectively; a CDR-1, a CDR-2, and a CDR-3, comprising SEQ ID NOs:9, 10, 258, respectively; a CDR-1, a CDR-2, and a CDR-3, comprising SEQ ID NOs:9, 10, 158, respectively; or a CDR-1, a CDR-2, and a CDR-3, comprising SEQ ID NOs:9, 10, 284, respectively. Also among the provided TCRs are those having sequences at least at or about 90%, 91%, 92%, 93%, 94%, 95%, 96%, 97%, 98%, or 99% identical to such sequences.

In some embodiments, a TCR or antigen-binding fragment thereof provided herein includes a Vα or Vγ region that contains a CDR-1, a CDR-2, and a CDR-3, comprising a CDR-1, a CDR-2, and a CDR-3 amino acid sequence, respectively, set forth in Table 1, such as in each row therein and a Vβ or Vδ region that contains a CDR-1, a CDR-2, and a CDR-3, comprising a CDR-1, a CDR-2, and a CDR-3 amino acid sequence, respectively, set forth in Table 1, such as in each row therein. In some embodiments, a TCR or antigen-binding fragment thereof provided herein includes a Vα or Vγ region that contains a CDR-1, a CDR-2, and a CDR-3, comprising a CDR-1, a CDR-2, and a CDR-3 amino acid sequence, respectively, contained within a Vα or Vγ region amino acid sequence set forth in Table 1, such as in each row therein, and a Vβ or Vδ region that contains a CDR-1, a CDR-2, and a CDR-3, comprising a CDR-1, a CDR-2, and a CDR-3 amino acid sequence, respectively, contained within a Vα or Vγ region amino acid sequence set forth in Table 1, such as in each row therein. Also among the provided TCRs are those containing sequences at least at or about 90%, 91%, 92%, 93%, 94%, 95%, 96%, 97%, 98%, or 99% identical to such sequences. Exemplary TCRs containing such CDRs, or their modified versions as described elsewhere herein, also are set forth in the Table 1, such as in each row therein.

TABLE 1
SEQ ID NOs of Amino Acid Sequences of CDRs
and Variable Regions of HA-1 Specific TCRs

| | Alpha variable | | | | Beta variable | | | |
|---|---|---|---|---|---|---|---|---|
| | Variable | | | | Variable | | | |
| TCR | Region | CDR-1 | CDR-2 | CDR-3 | Region | CDR-1 | CDR-2 | CDR-3 |
| TCR A | 4 | 1 | 2 | 3 | 12 | 9 | 10 | 11 |
| TCR B | 22 | 19 | 20 | 21 | 28 | 9 | 10 | 27 |
| TCR C | 38 | 35 | 36 | 37 | 44 | 9 | 10 | 43 |
| TCR D | 52 | 1 | 2 | 51 | 58 | 9 | 10 | 57 |
| TCR E | 66 | 1 | 2 | 65 | 58 | 9 | 10 | 57 |
| TCR F | 79 | 76 | 77 | 78 | 85 | 9 | 10 | 84 |
| TCR G | 93 | 19 | 20 | 92 | 99 | 9 | 10 | 98 |
| TCR H | 107 | 76 | 77 | 106 | 113 | 9 | 10 | 112 |
| TCR I | 121 | 35 | 36 | 120 | 127 | 9 | 10 | 126 |
| TCR J | 137 | 134 | 135 | 136 | 143 | 9 | 10 | 142 |
| TCR K | 153 | 150 | 151 | 152 | 159 | 9 | 10 | 158 |
| TCR L | 167 | 35 | 36 | 166 | 173 | 9 | 10 | 172 |
| TCR M | 181 | 35 | 36 | 180 | 187 | 9 | 10 | 186 |
| TCR N | 195 | 76 | 77 | 194 | 201 | 9 | 10 | 200 |
| TCR O | 209 | 76 | 77 | 208 | 215 | 9 | 10 | 214 |
| TCR P | 225 | 222 | 223 | 224 | 231 | 9 | 10 | 230 |
| TCR Q | 239 | 76 | 77 | 238 | 245 | 9 | 10 | 244 |
| TCR R | 253 | 19 | 20 | 252 | 259 | 9 | 10 | 258 |
| TCR S | 269 | 266 | 267 | 268 | 159 | 9 | 10 | 158 |
| TCR T | 279 | 19 | 20 | 278 | 285 | 9 | 10 | 284 |
| TCR U | 360 | a | b | 359 | 364 | 9 | 10 | 363 |
| TCR V | 370 | a | b | 369 | 374 | 9 | 10 | 373 |
| TCR W | 380 | a | b | 379 | 384 | 9 | 10 | 383 |
| TCR X | 390 | a | b | 389 | 394 | 9 | 10 | 393 |
| TCR Y | 400 | a | b | 399 | 404 | 9 | 10 | 403 |
| TCR Z | 410 | a | b | 409 | 414 | 9 | 10 | 413 |
| TCR AA | 420 | a | b | 419 | 424 | 9 | 10 | 423 |
| TCR AB | 430 | a | b | 429 | 434 | 9 | 10 | 433 |
| TCR AC | 440 | a | b | 439 | 444 | 9 | 10 | 443 |
| TCR AD | 450 | a | b | 449 | 454 | 9 | 10 | 453 |
| TCR AE | | a | b | 470 | | 9 | 10 | 57 |
| TCR AF | | a | b | 471 | | 9 | 10 | 57 |
| TCR AG | | a | b | 472 | | 9 | 10 | 57 |
| TCR AH | | a | b | 473 | | 9 | 10 | 57 |
| TCR AI | | a | b | 474 | | 9 | 10 | 475 |
| TCR AJ | 481 | a | b | 480 | 485 | 490 | 491 | 484 |

a can be any of the CDR1 Vα sequences shown in Table 1
b can be any of the CDR2 Vα sequences shown in Table 1

In some examples, a TCR or antigen binding fragment thereof provided herein can be designed to include an alpha chain (or gamma chain) that includes a set of three CDRs (e.g., a CDR-1, CDR-2, and CDR-3) as set forth in Table 1 (e.g., SEQ ID NOs: 1-3; SEQ ID NOs: 19-21; SEQ ID NOs:35-37; SEQ ID NOs:1, 2, and 51; SEQ ID NOs:1, 2, and 65; SEQ ID NOs: 76-78; SEQ ID NOs: 19, 20, and 92; SEQ ID NOs: 76, 77, and 106; SEQ ID NOs:35, 36, and 120; SEQ ID NOs:134-136; SEQ ID NOs:150-152; SEQ ID NOs:35, 36, and 166; SEQ ID NOs:35, 36, and 180; SEQ ID NOs: 76, 77, and 194; SEQ ID NOs: 76, 77, and 208; SEQ ID NOs:222-224; SEQ ID NOs: 76, 77, and 238; SEQ ID NOs:19, 20, and 252; SEQ ID NOs:266-268; or SEQ ID NOs:19, 20, and 278) and a beta chain (or delta chain) that includes a set of three CDRs (e.g., a CDR-1, CDR-2, and CDR-3) as set forth in Table 1 (e.g., SEQ ID NOs:9-10; SEQ ID NOs:9, 10, and 27; SEQ ID NOs:9, 10, and 43; SEQ ID NOs: 9, 10, and 57; SEQ ID NOs: 9, 10, and 84; SEQ ID NOs:9, 10, and 98; SEQ ID NOs:9, 10, and 112; SEQ ID NOs:9, 10, and 126; SEQ ID NOs:9, 10, and 142; SEQ ID NOs:9, 10, and 158; SEQ ID NOs:9, 10, and 172; SEQ ID NOs:9, 10, and 186; SEQ ID NOs:9, 10, and 200; SEQ ID NOs:9, 10, and 214; SEQ ID NOs:9, 10, and 230; SEQ ID NOs:9, 10, and 244; SEQ ID NOs:9, 10, and 258; or SEQ ID NOs:9, 10, and 284).

In some of any of the embodiments provided herein, the Vα region comprises a CDR-1 comprising SEQ ID NO:1, a CDR-2 comprising SEQ ID NO:2, and a CDR-3 comprising SEQ ID NO:3, and the Vβ region comprises a CDR-1 comprising SEQ ID NO:9, a CDR-2 comprising SEQ ID NO:10, and a CDR-3 comprising SEQ ID NO:11. In some of any of the embodiments provided herein, the Vα region comprises a CDR-1 comprising SEQ ID NO:19, a CDR-2 comprising SEQ ID NO:20, and a CDR-3 comprising SEQ ID NO:21, and the Vβ region comprises a CDR-1 comprising SEQ ID NO:9, a CDR-2 comprising SEQ ID NO:10, and a CDR-3 comprising SEQ ID NO:27. In some of any of the embodiments provided herein, the Vα region comprises a CDR-1 comprising SEQ ID NO:35, a CDR-2 comprising SEQ ID NO:36, and a CDR-3 comprising SEQ ID NO:37, and the Vβ region comprises a CDR-1 comprising SEQ ID NO:9, a CDR-2 comprising SEQ ID NO: 10, and a CDR-3 comprising SEQ ID NO:43. In some of any of the embodiments provided herein, the Vα region comprises a CDR-1 comprising SEQ ID NO:1, a CDR-2 comprising SEQ ID NO:2, and a CDR-3 comprising SEQ ID NO:51, and the Vβ region comprises a CDR-1 comprising SEQ ID NO:9, a CDR-2 comprising SEQ ID NO:10, and a CDR-3 comprising SEQ ID NO:57. In some of any of the embodiments provided herein, the Vα region comprises a CDR-1 comprising SEQ ID NO:1, a CDR-2 comprising SEQ ID NO:2, and a CDR-3 comprising SEQ ID NO:65, and the Vβ region comprises a CDR-1 comprising SEQ ID NO:9, a CDR-2 comprising SEQ ID NO: 10, and a CDR-3 comprising SEQ ID NO:57. In some of any of the embodiments provided herein, the Vα region comprises a CDR-1 comprising SEQ ID NO:76, a CDR-2 comprising SEQ ID NO:77, and a CDR-3 comprising SEQ ID NO: 78, and the Vβ region comprises a CDR-1 comprising SEQ ID NO:9, a CDR-2 comprising SEQ ID NO:10, and a CDR-3 comprising SEQ ID NO:84. In some of any of the embodiments provided herein, the Vα region comprises a CDR-1 comprising SEQ ID NO:19, a CDR-2 comprising SEQ ID NO:20, and a CDR-3 comprising SEQ ID NO:92, and the Vβ region comprises a CDR-1 comprising SEQ ID NO:9, a CDR-2 comprising SEQ ID NO:10, and a CDR-3 comprising SEQ ID NO:98. In some of any of the embodiments provided herein, the Vα region comprises a CDR-1 comprising SEQ ID NO:76, a CDR-2 comprising SEQ ID NO:77, and a CDR-3 comprising SEQ ID NO:106, and the Vβ region comprises a CDR-1 comprising SEQ ID NO:9, a CDR-2 comprising SEQ ID NO:10, and a CDR-3 comprising SEQ ID NO:112. In some of any of the embodiments provided herein, the Vα region comprises a CDR-1 comprising SEQ ID NO:35, a CDR-2 comprising SEQ ID NO:36, and a CDR-3 comprising SEQ ID NO:120, and the Vβ region comprises a CDR-1 comprising SEQ ID NO:9, a CDR-2 comprising SEQ ID NO: 10, and a CDR-3 comprising SEQ ID NO:126. In some of any of the embodiments provided herein, the Vα region comprises a CDR-1 comprising SEQ ID NO: 134, a CDR-2 comprising SEQ ID NO: 135, and a CDR-3 comprising SEQ ID NO:136, and the Vβ region comprises a CDR-1 comprising SEQ ID NO:9, a CDR-2 comprising SEQ ID NO:10, and a CDR-3 comprising SEQ ID NO: 142. In some of any of the embodiments provided herein, the Vα region comprises a CDR-1 comprising SEQ ID NO: 150, a CDR-2 comprising SEQ ID NO:151, and a CDR-3 comprising SEQ ID NO: 152, and the Vβ region comprises a CDR-1 comprising SEQ ID NO:9, a CDR-2 comprising SEQ ID NO:10, and a CDR-3 comprising SEQ ID NO:158. In some of any of the embodiments provided herein, the Vα region comprises a CDR-1 comprising SEQ ID NO:35, a CDR-2 comprising SEQ ID NO:36, and a CDR-3 comprising SEQ ID NO:166, and the Vβ region comprises a CDR-1 comprising SEQ ID NO:9, a CDR-2 comprising SEQ ID NO:10, and a CDR-3 comprising SEQ ID NO:172. In some of any of the embodiments provided herein, the Vα region comprises a CDR-1 comprising SEQ ID NO:35, a CDR-2 comprising SEQ ID NO:36, and a CDR-3 comprising SEQ ID NO: 180, and the Vβ region comprises a CDR-1 comprising SEQ ID NO:9, a CDR-2 comprising SEQ ID NO:10, and a CDR-3 comprising SEQ ID NO:186. In some of any of the embodiments provided herein, the Vα region comprises a CDR-1 comprising SEQ ID NO:76, a CDR-2 comprising SEQ ID NO:77, and a CDR-3 comprising SEQ ID NO: 194, and the Vβ region comprises a CDR-1 comprising SEQ ID NO:9, a CDR-2 comprising SEQ ID NO: 10, and a CDR-3 comprising SEQ ID NO:200. In some of any of the embodiments provided herein, the Vα region comprises a CDR-1 comprising SEQ ID NO:76, a CDR-2 comprising SEQ ID NO:77, and a CDR-3 comprising SEQ ID NO:208, and the Vβ region comprises a CDR-1 comprising SEQ ID NO:9, a CDR-2 comprising SEQ ID NO:10, and a CDR-3 comprising SEQ ID NO:214. In some of any of the embodiments provided herein, the Vα region comprises a CDR-1 comprising SEQ ID NO:222, a CDR-2 comprising SEQ ID NO:223, and a CDR-3 comprising SEQ ID NO:224, and the Vβ region comprises a CDR-1 comprising SEQ ID NO:9, a CDR-2 comprising SEQ ID NO:10, and a CDR-3 comprising SEQ ID NO:230. In some of any of the embodiments provided herein, the Vα region comprises a CDR-1 comprising SEQ ID NO:76, a CDR-2 comprising SEQ ID NO:77, and a CDR-3 comprising SEQ ID NO:238, and the Vβ region comprises a CDR-1 comprising SEQ ID NO:9, a CDR-2 comprising SEQ ID NO: 10, and a CDR-3 comprising SEQ ID NO:244. In some of any of the embodiments provided herein, the Vα region comprises a CDR-1 comprising SEQ ID NO:19, a CDR-2 comprising SEQ ID NO:20, and a CDR-3 comprising SEQ ID NO:252, and the Vβ region comprises a CDR-1 comprising SEQ ID NO:9, a CDR-2 comprising SEQ ID NO:10, and a CDR-3 comprising SEQ ID NO:258. In some of any of the embodiments provided herein, the Vα region comprises a CDR-1 comprising SEQ ID NO:266, a CDR-2 comprising SEQ ID NO:267, and a CDR-3 comprising SEQ ID NO:268, and the Vβ region comprises a CDR-1 comprising SEQ ID NO:9, a CDR-2 comprising SEQ ID NO: 10, and a CDR-3 comprising SEQ ID NO: 158. In some of any of the embodiments provided herein, the Vα region comprises a CDR-1 comprising SEQ ID NO:19, a CDR-2 comprising SEQ ID NO:20, and a CDR-3 comprising SEQ ID NO:278, and the Vβ region comprises a CDR-1 comprising SEQ ID NO:9, a CDR-2 comprising SEQ ID NO:10, and a CDR-3 comprising SEQ ID NO:284.

In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:4, and the Vβ region comprises a CDR-1, a CDR-2, and a CDR-3 comprising a CDR-1, a CDR-2, and a CDR-3, respectively, contained within the Vβ region sequence of SEQ ID NO:12. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:22, and the Vβ region comprises a CDR-1, a CDR-2, and a CDR-3 comprising a CDR-1, a CDR-2, and a CDR-3, respectively, contained within the Vβ region sequence of SEQ ID NO:28. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:38, and the Vβ region comprises a CDR-1, a CDR-2, and a CDR-3 comprising a CDR-1, a CDR-2, and a CDR-3, respectively, contained within the Vβ region sequence of SEQ ID NO:44. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:52, and the Vβ region comprises a CDR-1, a CDR-2, and a CDR-3 comprising a CDR-1, a CDR-2, and a CDR-3, respectively, contained within the Vβ region sequence of SEQ ID NO:58. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:66, and the Vβ region comprises a CDR-1, a CDR-2, and a CDR-3 comprising a CDR-1, a CDR-2, and a CDR-3, respectively, contained within the Vβ region sequence of SEQ ID NO:58. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:79, and the Vβ region comprises a CDR-1, a CDR-2, and a CDR-3 comprising a CDR-1, a CDR-2, and a CDR-3, respectively, contained within the Vβ region sequence of SEQ ID NO:85. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:93, and the Vβ region comprises a CDR-1, a CDR-2, and a CDR-3 comprising a CDR-1, a CDR-2, and a CDR-3, respectively, contained within the Vβ region sequence of SEQ ID NO:99. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:107, and the Vβ region comprises a CDR-1, a CDR-2, and a CDR-3 comprising a CDR-1, a CDR-2, and a CDR-3, respectively, contained within the Vβ region sequence of SEQ ID NO: 113. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO: 121, and the Vβ region comprises a CDR-1, a CDR-2, and a CDR-3 comprising a CDR-1, a CDR-2, and a CDR-3, respectively, contained within the Vβ region sequence of SEQ ID NO: 127. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:137, and the Vβ region comprises a CDR-1, a CDR-2, and a CDR-3 comprising a CDR-1, a CDR-2, and a CDR-3, respectively, contained within the Vβ region sequence of SEQ ID NO:143. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:153, and the Vβ region comprises a CDR-1, a CDR-2, and a CDR-3 comprising a CDR-1, a CDR-2, and a CDR-3, respectively, contained within the Vβ region sequence of SEQ ID NO: 159. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO: 167, and the Vβ region comprises a CDR-1, a CDR-2, and a CDR-3 comprising a CDR-1, a CDR-2, and a CDR-3, respectively, contained within the Vβ region sequence of SEQ ID NO: 173. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO: 181, and the Vβ region comprises a CDR-1, a CDR-2, and a CDR-3 comprising a CDR-1, a CDR-2, and a CDR-3, respectively, contained within the Vβ region sequence of SEQ ID NO: 187. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:195, and the Vβ region comprises a CDR-1, a CDR-2, and a CDR-3 comprising a CDR-1, a CDR-2, and a CDR-3, respectively, contained within the Vβ region sequence of SEQ ID NO:201. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:209, and the Vβ region comprises a CDR-1, a CDR-2, and a CDR-3 comprising a CDR-1, a CDR-2, and a CDR-3, respectively, contained within the Vβ region sequence of SEQ ID NO:215. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:225, and the Vβ region comprises a CDR-1, a CDR-2, and a CDR-3 comprising a CDR-1, a CDR-2, and a CDR-3, respectively, contained within the Vβ region sequence of SEQ ID NO:231. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:239, and the Vβ region comprises a CDR-1, a CDR-2, and a CDR-3 comprising a CDR-1, a CDR-2, and a CDR-3, respectively, contained within the Vβ region sequence of SEQ ID NO:245. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:253, and the Vβ region comprises a CDR-1, a CDR-2, and a CDR-3 comprising a CDR-1, a CDR-2, and a CDR-3, respectively, contained within the Vβ region sequence of SEQ ID NO:259. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:269, and the Vβ region comprises a CDR-1, a CDR-2, and a CDR-3 comprising a CDR-1, a CDR-2, and a CDR-3, respectively, contained within the Vβ region sequence of SEQ ID NO:159. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:279, and the Vβ region comprises a CDR-1, a CDR-2, and a CDR-3 comprising a CDR-1, a CDR-2, and a CDR-3, respectively, contained within the Vβ region sequence of SEQ ID NO:285. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:360, and the Vβ region comprises a CDR-1, a CDR-2, and a CDR-3 comprising a CDR-1, a CDR-2, and a CDR-3, respectively, contained within the Vβ region sequence of SEQ ID NO:364. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:370, and the Vβ region comprises a CDR-1, a CDR-2, and a CDR-3 comprising a CDR-1, a CDR-2, and a CDR-3, respectively, contained within the Vβ region sequence of SEQ ID NO:374. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:380, and the Vβ region comprises a CDR-1, a CDR-2, and a CDR-3 comprising a CDR-1, a CDR-2, and a CDR-3, respectively, contained within the Vβ region sequence of SEQ ID NO:384. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:390, and the Vβ region comprises a CDR-1, a CDR-2, and a CDR-3 comprising a CDR-1, a CDR-2, and a CDR-3, respectively, contained within the Vβ region sequence of SEQ ID NO:394. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:400, and the Vβ region comprises a CDR-1, a CDR-2, and a CDR-3 comprising a CDR-1, a CDR-2, and a CDR-3, respectively, contained within the Vβ region sequence of SEQ ID NO:404. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:410, and the Vβ region comprises a CDR-1, a CDR-2, and a CDR-3 comprising a CDR-1, a CDR-2, and a CDR-3, respectively, contained within the Vβ region sequence of SEQ ID NO:414. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:420, and the Vβ region comprises a CDR-1, a CDR-2, and a CDR-3 comprising a CDR-1, a CDR-2, and a CDR-3, respectively, contained within the Vβ region sequence of SEQ ID NO:424. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:430 and the Vβ region comprises a CDR-1, a CDR-2, and a CDR-3 comprising a CDR-1, a CDR-2, and a CDR-3, respectively, contained within the Vβ region sequence of SEQ ID NO:434. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO:440, and the Vβ region comprises a CDR-1, a CDR-2, and a CDR-3 comprising a CDR-1, a CDR-2, and a CDR-3, respectively, contained within the Vβ region sequence of SEQ ID NO:444. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO: 450 and the Vβ region comprises a CDR-1, a CDR-2, and a CDR-3 comprising a CDR-1, a CDR-2, and a CDR-3, respectively, contained within the Vβ region sequence of SEQ ID NO:454. In some of any of the embodiments provided herein, the Vα region comprises a CDR-1, a CDR-2, and a CDR-3 comprising a CDR-1, a CDR-2, and a CDR-3, respectively, contained within the Vα region sequence of SEQ ID NO: 481 and the Vβ region comprises a CDR-1, a CDR-2, and a CDR-3 comprising a CDR-1, a CDR-2, and a CDR-3, respectively, contained within the Vβ region sequence of SEQ ID NO:485.

In some of any of the embodiments provided herein, the Vα region comprises SEQ ID NO:4 or a sequence that has at least 90% sequence identity thereto, and the Vβ region comprises SEQ ID NO:12 or a sequence that has at least 90% sequence identity thereto. In some of any of the embodiments provided herein, the Vα region comprises SEQ ID NO:22 or a sequence that has at least 90% sequence identity thereto, and the Vβ region comprises SEQ ID NO:28 or a sequence that has at least 90% sequence identity thereto. In some of any of the embodiments provided herein, the Vα region comprises SEQ ID NO:38 or a sequence that has at least 90% sequence identity thereto, and the Vβ region comprises SEQ ID NO:44 or a sequence that has at least 90% sequence identity thereto. In some of any of the embodiments provided herein, the Vα region comprises SEQ ID NO:52 or a sequence that has at least 90% sequence identity thereto, and the Vβ region comprises SEQ ID NO:58 or a sequence that has at least 90% sequence identity thereto. In some of any of the embodiments provided herein, the Vα region comprises SEQ ID NO:66 or a sequence that has at least 90% sequence identity thereto, and the Vβ region comprises SEQ ID NO:58 or a sequence that has at least 90% sequence identity thereto. In some of any of the embodiments provided herein, the Vα region comprises SEQ ID NO:79 or a sequence that has at least 90% sequence identity thereto, and the Vβ region comprises SEQ ID NO:85 or a sequence that has at least 90% sequence identity thereto. In some of any of the embodiments provided herein, the Vα region comprises SEQ ID NO:93 or a sequence that has at least 90% sequence identity thereto, and the Vβ region comprises SEQ ID NO:99 or a sequence that has at least 90% sequence identity thereto. In some of any of the embodiments provided herein, the Vα region comprises SEQ ID NO:107 or a sequence that has at least 90% sequence identity thereto, and the Vβ region comprises SEQ ID NO:113 or a sequence that has at least 90% sequence identity thereto. In some of any of the embodiments provided herein, the Vα region comprises SEQ ID NO: 121 or a sequence that has at least 90% sequence identity thereto, and the Vβ region comprises SEQ ID NO: 127 or a sequence that has at least 90% sequence identity thereto. In some of any of the embodiments provided herein, the Vα region comprises SEQ ID NO:137 or a sequence that has at least 90% sequence identity thereto, and the Vβ region comprises SEQ ID NO: 143 or a sequence that has at least 90% sequence identity thereto. In some of any of the embodiments provided herein, the Vα region comprises SEQ ID NO:153 or a sequence that has at least 90% sequence identity thereto, and the Vβ region comprises SEQ ID NO: 159 or a sequence that has at least 90% sequence identity thereto. In some of any of the embodiments provided herein, the Vα region comprises SEQ ID NO:167 or a sequence that has at least 90% sequence identity thereto, and the Vβ region comprises SEQ ID NO: 173 or a sequence that has at least 90% sequence identity thereto. In some of any of the embodiments provided herein, the Vα region comprises SEQ ID NO:181 or a sequence that has at least 90% sequence identity thereto, and the Vβ region comprises SEQ ID NO:187 or a sequence that has at least 90% sequence identity thereto. In some of any of the embodiments provided herein, the Vα region comprises SEQ ID NO: 195 or a sequence that has at least 90% sequence identity thereto, and the Vβ region comprises SEQ ID NO:201 or a sequence that has at least 90% sequence identity thereto. In some of any of the embodiments provided herein, the Vα region comprises SEQ ID NO:209 or a sequence that has at least 90% sequence identity thereto, and the Vβ region comprises SEQ ID NO:215 or a sequence that has at least 90% sequence identity thereto. In some of any of the embodiments provided herein, the Vα region comprises SEQ ID NO:225 or a sequence that has at least 90% sequence identity thereto, and the Vβ region comprises SEQ ID NO:231 or a sequence that has at least 90% sequence identity thereto. In some of any of the embodiments provided herein, the Vα region comprises SEQ ID NO:239 or a sequence that has at least 90% sequence identity thereto, and the Vβ region comprises SEQ ID NO:245 or a sequence that has at least 90% sequence identity thereto. In some of any of the embodiments provided herein, the Vα region comprises SEQ ID NO:253 or a sequence that has at least 90% sequence identity thereto, and the Vβ region comprises SEQ ID NO:259 or a sequence that has at least 90% sequence identity thereto. In some of any of the embodiments provided herein, the Vα region comprises SEQ ID NO:269 or a sequence that has at least 90% sequence identity thereto, and the Vβ region comprises SEQ ID NO:159 or a sequence that has at least 90% sequence identity thereto. In some of any of the embodiments provided herein, the Vα region comprises SEQ ID NO:279 or a sequence that has at least 90% sequence identity thereto, and the Vβ region comprises SEQ ID NO:285 or a sequence that has at least 90% sequence identity thereto. In some of any of the embodiments provided herein, the Vα region comprises SEQ ID NO:360 or a sequence that has at least 90% sequence identity thereto, and the Vβ region comprises SEQ ID NO:364 or a sequence that has at least 90% sequence identity thereto. In some of any of the embodiments provided herein, the Vα region comprises SEQ ID NO:370 or a sequence that has at least 90% sequence identity thereto, and the Vβ region comprises SEQ ID NO:374 or a sequence that has at least 90% sequence identity thereto. In some of any of the embodiments provided herein, the Vα region comprises SEQ ID NO:380 or a sequence that has at least 90% sequence identity thereto, and the Vβ region comprises SEQ ID NO:384 or a sequence that has at least 90% sequence identity thereto. In some of any of the embodiments provided herein, the Vα region comprises SEQ ID NO:390 or a sequence that has at least 90% sequence identity thereto, and the Vβ region comprises SEQ ID NO:394 or a sequence that has at least 90% sequence identity thereto. In some of any of the embodiments provided herein, the Vα region comprises SEQ ID NO:400 or a sequence that has at least 90% sequence identity thereto, and the Vβ region comprises SEQ ID NO:404 or a sequence that has at least 90% sequence identity thereto. In some of any of the embodiments provided herein, the Vα region comprises SEQ ID NO:410 or a sequence that has at least 90% sequence identity thereto, and the Vβ region comprises SEQ ID NO:414 or a sequence that has at least 90% sequence identity thereto. In some of any of the embodiments provided herein, the Vα region comprises SEQ ID NO:420 or a sequence that has at least 90% sequence identity thereto, and the Vβ region comprises SEQ ID NO:424 or a sequence that has at least 90% sequence identity thereto. In some of any of the embodiments provided herein, the Vα region comprises SEQ ID NO:430 or a sequence that has at least 90% sequence identity thereto, and the Vβ region comprises SEQ ID NO:434 or a sequence that has at least 90% sequence identity thereto. In some of any of the embodiments provided herein, the Vα region comprises SEQ ID NO:440 or a sequence that has at least 90% sequence identity thereto, and the Vβ region comprises SEQ ID NO:444 or a sequence that has at least 90% sequence identity thereto. In some of any of the embodiments provided herein, the Vα region comprises SEQ ID NO:450 or a sequence that has at least 90% sequence identity thereto, and the Vβ region comprises SEQ ID NO:454 or a sequence that has at least 90% sequence identity thereto. In some of any of the embodiments provided herein, the Vα region comprises SEQ ID NO:481 or a sequence that has at least 90% sequence identity thereto, and the Vβ region comprises SEQ ID NO:485 or a sequence that has at least 90% sequence identity thereto.

In some of any of the embodiments provided herein, the Vα region comprises SEQ ID NO:4, and the Vβ region comprises SEQ ID NO:12. In some of any of the embodiments provided herein, the Vα region comprises SEQ ID NO:22, and the Vβ region comprises SEQ ID NO:28. In some of any of the embodiments provided herein, the Vα region comprises SEQ ID NO:38, and the Vβ region comprises SEQ ID NO:44. In some of any of the embodiments provided herein, the Vα region comprises SEQ ID NO:52, and the Vβ region comprises SEQ ID NO:58. In some of any of the embodiments provided herein, the Vα region comprises SEQ ID NO:66, and the Vβ region comprises SEQ ID NO:58. In some of any of the embodiments provided herein, the Vα region comprises SEQ ID NO:79, and the Vβ region comprises SEQ ID NO:85. In some of any of the embodiments provided herein, the Vα region comprises SEQ ID NO:93, and the Vβ region comprises SEQ ID NO:99. In some of any of the embodiments provided herein, the Vα region comprises SEQ ID NO:107, and the Vβ region comprises SEQ ID NO: 113. In some of any of the embodiments provided herein, the Vα region comprises SEQ ID NO:121, and the Vβ region comprises SEQ ID NO:127. In some of any of the embodiments provided herein, the Vα region comprises SEQ ID NO:137, and the Vβ region comprises SEQ ID NO: 143. In some of any of the embodiments provided herein, the Vα region comprises SEQ ID NO: 153, and the Vβ region comprises SEQ ID NO: 159. In some of any of the embodiments provided herein, the Vα region comprises SEQ ID NO:167, and the Vβ region comprises SEQ ID NO: 173. In some of any of the embodiments provided herein, the Vα region comprises SEQ ID NO: 181, and the Vβ region comprises SEQ ID NO: 187. In some of any of the embodiments provided herein, the Vα region comprises SEQ ID NO: 195, and the Vβ region comprises SEQ ID NO:201. In some of any of the embodiments provided herein, the Vα region comprises SEQ ID NO:209, and the Vβ region comprises SEQ ID NO:215. In some of any of the embodiments provided herein, the Vα region comprises SEQ ID NO:225, and the Vβ region comprises SEQ ID NO:231. In some of any of the embodiments provided herein, the Vα region comprises SEQ ID NO:239, and the Vβ region comprises SEQ ID NO:245. In some of any of the embodiments provided herein, the Vα region comprises SEQ ID NO:253, and the Vβ region comprises SEQ ID NO:259. In some of any of the embodiments provided herein, the Vα region comprises SEQ ID NO:269, and the Vβ region comprises SEQ ID NO: 159. In some of any of the embodiments provided herein, the Vα region comprises SEQ ID NO:279, and the Vβ region comprises SEQ ID NO:285. In some of any of the embodiments provided herein, the Vα region comprises SEQ ID NO:360, and the Vβ region comprises SEQ ID NO:364. In some of any of the embodiments provided herein, the Vα region comprises SEQ ID NO:370, and the Vβ region comprises SEQ ID NO:374. In some of any of the embodiments provided herein, the Vα region comprises SEQ ID NO:380, and the Vβ region comprises SEQ ID NO:384. In some of any of the embodiments provided herein, the Vα region comprises SEQ ID NO:390, and the Vβ region comprises SEQ ID NO:394. In some of any of the embodiments provided herein, the Vα region comprises SEQ ID NO:400, and the Vβ region comprises SEQ ID NO:404. In some of any of the embodiments provided herein, the Vα region comprises SEQ ID NO:410, and the Vβ region comprises SEQ ID NO:414. In some of any of the embodiments provided herein, the Vα region comprises SEQ ID NO:420, and the Vβ region comprises SEQ ID NO:424. In some of any of the embodiments provided herein, the Vα region comprises SEQ ID NO:430, and the Vβ region comprises SEQ ID NO:434. In some of any of the embodiments provided herein, the Vα region comprises SEQ ID NO:440, and the Vβ region comprises SEQ ID NO:444. In some of any of the embodiments provided herein, the Vα region comprises SEQ ID NO: 450, and the Vβ region comprises SEQ ID NO:454. In some of any of the embodiments provided herein, the Vα region comprises SEQ ID NO: 481, and the Vβ region comprises SEQ ID NO:485.

### D. Exemplary Constant Domains

In some embodiments, the alpha chain of a TCR or antigen-binding fragment thereof provided herein further contains an alpha constant (Cα) region or portion thereof. In some aspects, the beta chain further contains a beta constant (Cβ) region or portion thereof. Thus, in some embodiments, a TCR provided herein (e.g., an anti-HA-1 TCR provided herein) or an antigen-binding fragment thereof contains an alpha chain comprising a Vα region and a Cα domain or portion thereof and/or a beta chain comprising a Vβ region and a Cβ domain or portion thereof. In some embodiments, the gamma chain of a TCR or antigen-binding fragment thereof provided herein further contains a gamma constant (Cδ) region or portion thereof. In some aspects, the delta chain further contains a delta constant (Cδ) region or portion thereof. Thus, in some embodiments, a TCR provided herein (e.g., an anti-HA-1 TCR provided herein) or an antigen-binding fragment thereof contains a gamma chain comprising a Vγ region and a Cy domain or portion thereof and/or a delta chain comprising a Vδ region and a Cδ domain or portion thereof.

In some embodiments, the α chain and the β chain, or the γ chain and the δ chain, of a TCR provided herein each further contain a constant domain. In some embodiments, the Cα domain and Cβ domain, or the Cγ domain and Cδ domain, individually are mammalian (e.g., a human or murine constant domain). In some embodiments, the constant domain is adjacent to the cell membrane. For example, in some cases, the extracellular portion of the TCR formed by the two chains contains two membrane-proximal constant domains, and two membrane-distal variable domains, which variable domains each contain CDRs.

In some aspects, provided herein are TCRs that contains a human constant domain, such as an alpha chain containing a human Cα domain and a beta chain containing a human Cβ domain, or a gamma chain containing a human Cγ domain and a delta chain containing a human Cδ domain. In some embodiments, the provided TCRs are fully human. Among the provided TCRs are TCRs containing a human constant domain, such as fully human TCRs, whose expression and/or activity, such as when expressed in human cells, e.g. human T cells, such as primary human T cells, are not impacted by or are not substantially impacted by the presence of an endogenous human TCR.

In some embodiments, each of the Cα and the Cβ domains, or each of the Cγ and the C8 domains, is human. In some embodiments, the Cα is encoded by the TRAC gene (IMGT nomenclature) or is a variant thereof. In some embodiments, the Cβ is encoded by TRBC1 or TRBC2 genes (IMGT nomenclature) or is a variant thereof. In some embodiments, the Cγ is encoded by the TRGC1 or TRGC2 genes (IMGT nomenclature) or is a variant thereof. In some embodiments, the C8 is encoded by TRDC genes (IMGT nomenclature) or is a variant thereof.

In some embodiments, the Cα domain or a variant thereof has or comprises the sequence of amino acids set forth in SEQ ID NO: 294 or 296, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 294 or 296. In some embodiments, the Cα domain has or comprises the sequence of amino acids set forth in SEQ ID NO: 294. In some embodiments, the Cα domain has or comprises the sequence of amino acids set forth in SEQ ID NO: 296. In some embodiments, the Cβ domain or variant thereof has or comprises the sequence of amino acids set forth in SEQ ID NO:298 or 300, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 298 or 300. In some embodiments, the Cβ domain has or comprises the sequence of amino acids set forth in SEQ ID NO: 298. In some embodiments, the Cβ domain has or comprises the sequence of amino acids set forth in SEQ ID NO:300. In some embodiments, the TCR comprises a Cα domain and a Cβ domain set forth in SEQ ID NO:294 and 298, respectively. In some embodiments, the TCR comprises a Cα domain and a Cβ domain set forth in SEQ ID NO:296 and 298, respectively. In some embodiments, the TCR comprises a Cα domain and a Cβ domain set forth in SEQ ID NO:294 and 300, respectively. In some embodiments, the TCR comprises a Cα domain and a Cβ domain set forth in SEQ ID NO:296 and 300, respectively.

In some embodiments, the Cγ domain or a variant thereof has or comprises the sequence of amino acids set forth in SEQ ID NO: 356 or 358, or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 356 or 357. In some embodiments, the Cγ domain has or comprises the sequence of amino acids set forth in SEQ ID NO: 356. In some embodiments, the Cγ domain has or comprises the sequence of amino acids set forth in SEQ ID NO: 357. In some embodiments, the Cδ domain or variant thereof has or comprises the sequence of amino acids set forth in SEQ ID NO:358 or a sequence of amino acids that exhibits at least 85%, 86%, 87%, 88%, 89%, 90%, 91%, 92%, 93%, 94%, 95%, 96%, 97%, 98%, 99% or more sequence identity to SEQ ID NO: 358. In some embodiments, the TCR comprises a Cγ domain and a Cδ domain set forth in SEQ ID NO:356 and 358, respectively. In some embodiments, the TCR comprises a Cγ domain and a Cδ domain set forth in SEQ ID NO:357 and 358, respectively.

In some embodiments, the variant of a Cα domain contains replacement of at least one non-native cysteine, such as any replacement described herein. In some embodiments, the variant of a Cβ domain contains replacement of at least one non-native cysteine, such as any replacement described herein.

In some embodiments, any of the provided TCRs or antigen-binding fragments thereof can be a human/mouse chimeric TCR. In some cases, a TCR or antigen-binding fragment thereof provided herein comprises an alpha chain and/or a beta chain, or a gamma chain and/or a delta chain, comprising a mouse constant domain. In some embodiments, the Cα domain and/or the Cβ domain, or the Cγ domain and/or the Cδ domain, are a mouse Cα domain and/or a mouse Cβ domain, or a mouse Cγ domain and/or a mouse Cδ domain. In some embodiments, the Cα domain and/or the Cβ domain, or the Cγ domain and/or the Cδ domain, is or comprises any Cα domain and/or Cβ domain, or Cγ domain and/or Cδ domain described in WO 2015/184228, WO 2015/009604 and WO 2015/009606.

In some embodiments, a TCR or antigen-binding fragment thereof provided herein comprises a variant of an alpha chain and/or a beta chain, or a gamma chain and/or a delta chain. In some embodiments, the variant comprises the amino acid sequence of any of the TCRs described herein with one, two, three, or four or more amino acid substitution(s) in the constant domain of the alpha or beta chain. In some embodiments, the TCRs (or functional portions thereof) comprising the substituted amino acid sequence(s) advantageously provide one or more of decreased mis-pairing with an endogenous TCR chain(s), increased expression by a host cell, increased recognition of HA-1 targets, and increased anti-tumor activity as compared to the parent TCR comprising an unsubstituted amino acid sequence.

In some embodiments, the constant domain contains substituted amino acid sequences of the mouse constant domains of the TCR α and β chains, or TCR γ and δ chains corresponding with all or portions of the unsubstituted mouse Cα domain and mouse Cβ domain, or mouse Cγ domain and mouse Cδ domain. In some embodiments, the TCR may be a heterodimer of the α and β chains, or the γ and δ chains that are linked, such as by a disulfide bond or disulfide bonds. In some embodiments, the constant domain of the TCR may contain short connecting sequences in which a cysteine residue forms a disulfide bond, thereby linking the two chains of the TCR. In some embodiments, a TCR may have an additional cysteine residue in each of the α and β chains, or the γ and δ chains, such that the TCR contains two disulfide bonds in the constant domains. In some embodiments, each of the constant and variable domains contains disulfide bonds formed by cysteine residues.

In some embodiments, a TCR provided herein can contain an introduced disulfide bond or bonds. In some embodiments, the native disulfide bonds are not present. In some embodiments, the one or more of the native cysteines (e.g. in the constant domain of the α chain and the β chain, or the γ chain and the δ chain) that form a native interchain disulfide bond are substituted to another residue, such as to a serine or alanine. In some embodiments, an introduced disulfide bond can be formed by mutating non-cysteine residues on the alpha and beta chains, such as in the constant domain of the α chain and the β chain, or the γ chain and the δ chain, to cysteine. Opposing cysteines in the TCR α and β chains, or TCR γ and δ chains provide a disulfide bond that links the constant domains of TCR α and β chains, or TCR γ and δ chains of the substituted TCR to one another and which is not present in a TCR comprising the unsubstituted constant domain in which the native disulfide bonds are present, such as unsubstituted native human constant domain or the unsubstituted native mouse constant domain. In some embodiments, the presence of non-native cysteine residues (e.g. resulting in one or more non-native disulfide bonds) in a recombinant TCR can favor production of the desired recombinant TCR in a cell in which it is introduced over expression of a mismatched TCR pair containing a native TCR chain.

Exemplary non-native disulfide bonds of a TCR are described in published International PCT Patent Application No. WO2006/000830 and WO2006/037960. In some embodiments, cysteines can be introduced or substituted at a residue corresponding to Thr48 of the Cα domain and Ser57 of the Cβ domain, at residue Thr45 of the Cα domain and Ser77 of the Cβ domain, at residue Tyr 10 of the Cα domain and Ser17 of the Cβ domain, at residue Thr45 of the Cα domain and Asp59 of the Cβ domain and/or at residue Ser15 of the Cα domain and Glu15 of the Cβ domain.

In some embodiments, any of the provided cysteine mutations can be made at a corresponding position in another sequence, for example, in a human or mouse Cα domain and/or Cβ domain, or Cγ domain and/or Cδ domain, sequence described above. The term “corresponding” with reference to positions of a protein, such as recitation that amino acid positions “correspond to” amino acid positions in a disclosed sequence, such as set forth in the Sequence Listing, refers to amino acid positions identified upon alignment with the disclosed sequence based on structural sequence alignment or using a standard alignment algorithm, such as the GAP algorithm. For example, corresponding residues can be determined by alignment of a reference sequence with the Cα sequence set forth in any of SEQ ID NO: 294 or 296, or the Cβ sequence set forth in SEQ ID NO: 298 or 300, by structural alignment methods as described herein. By aligning the sequences, one can identify corresponding residues, for example, using conserved and identical amino acid residues as guides.

In some embodiments, a TCR or antigen-binding fragment thereof provided herein comprises an alpha or gamma chain that is or comprises the sequence of amino acids set forth in any of SEQ ID NOs: 6, 24, 40, 54, 68, 81, 95, 109, 123, 139, 155, 169, 183, 197, 211, 227, 241, 255, 271, 281, 362, 372, 382, 392, 402, 412, 422, 432, 442, 452 and 483, or a sequence that has at least 90% sequence identity thereto, such as a sequence having at least at or about 90%, 91%, 92%, 93%, 94%, 95%, 96%, 97%, 98%, or 99% identity with such a sequence and/or a beta or delta chain that is or comprises the sequence of amino acids set forth in SEQ ID NO: 14, 30, 46, 60, 71, 87, 101, 115, 129, 145, 161, 175, 189, 203, 217, 233, 247, 261, 161, 287, 366, 376, 386, 396, 406, 416, 426, 436, 446, 456, and 487, or a sequence that has at least 90% sequence identity thereto, such as a sequence having at least at or about 90%, 91%, 92%, 93%, 94%, 95%, 96%, 97%, 98%, or 99% identity with such a sequence.

Exemplary TCRs or antigen-binding fragments include those set forth in Table 2, such as in each row therein. In some embodiments, the Vα and Vβ region, or the Vγ and Vδ region, contain the amino acid sequences corresponding to the SEQ ID NOs: set forth in Table 2, such as in each row therein. In some embodiments, the Vα and Vβ region, or the Vγ and Vδ region, contain the CDR-1, the CDR-2 and the CDR-3 sequences contained within the Vα and Vβ region, or the Vγ and Vδ region, set forth in Table 2, such as in each row therein. In some aspects, the TCR contains constant alpha and constant beta domain sequences, such as those corresponding to the SEQ ID NOs: set forth in Table 2, such as in each row therein. In some cases, the TCR contains a full sequence comprising the variable and constant domains, such as a sequence corresponding to the SEQ ID NOs: set forth in Table 2 (“Full”), such as in each row therein. Also among the provided TCRs are those containing sequences at least at or about 90%, 91%, 92%, 93%, 94%, 95%, 96%, 97%, 98%, or 99% identical to such sequences. Exemplary TCRs containing such sequences, or their modified versions as described elsewhere herein, also are set forth in the Table 2, respectively, such as in each row therein. In some aspects, the provided exemplary TCRs, when expressed as a mature protein, comprises the mature Vα and/or mature Vβ region, or the mature Vγ and/or Vδ mature region, for example, without the signal sequence (e.g., from cleavage of the signal sequence) when fully processed and expressed.

TABLE 2
SEQ ID NOs of Amino Acid Sequences of Variable
and Constant Regions of HA-1 Specific TCRs

| | Alpha | | | Beta | | | |
|---|---|---|---|---|---|---|---|
| Exemplary | Variable | Constant | | Variable | Constant | | Full alpha- |
| TCR | (Vα) | (Cα) | Full | (Vβ) | (Cβ) | Full | P2A-beta |
| TCR A | 4 | 294 | 6 | 12 | 300 | 14 | 18 |
| TCR B | 22 | 294 | 24 | 28 | 298 | 30 | 34 |
| TCR C | 38 | 294 | 40 | 44 | 298 | 46 | 50 |
| TCR D | 52 | 294 | 54 | 58 | 300 | 60 | 64 |
| TCR E | 66 | 294 | 68 | 58 | 298 | 71 | 75 |
| TCR F | 79 | 296 | 81 | 85 | 300 | 87 | 91 |
| TCR G | 93 | 296 | 95 | 99 | 300 | 101 | 105 |
| TCR H | 107 | 294 | 109 | 113 | 300 | 115 | 119 |
| TCR I | 121 | 294 | 123 | 127 | 300 | 129 | 133 |
| TCR J | 137 | 294 | 139 | 143 | 300 | 145 | 149 |
| TCR K | 153 | 294 | 155 | 159 | 300 | 161 | 165 |
| TCR L | 167 | 294 | 169 | 173 | 298 | 175 | 179 |
| TCR M | 181 | 294 | 183 | 187 | 300 | 189 | 193 |
| TCR N | 195 | 296 | 197 | 201 | 300 | 203 | 207 |
| TCR O | 209 | 294 | 211 | 215 | 300 | 217 | 221 |
| TCR P | 225 | 294 | 227 | 231 | 300 | 233 | 237 |
| TCR Q | 239 | 296 | 241 | 245 | 298 | 247 | 251 |
| TCR R | 253 | 294 | 255 | 259 | 298 | 261 | 265 |
| TCR S | 269 | 296 | 271 | 159 | 300 | 161 | 277 |
| TCR T | 279 | 294 | 281 | 285 | 300 | 287 | 291 |
| TCR U | 360 | 294 | 362 | 364 | 300 | 366 | 367 |
| TCR V | 370 | 294 | 372 | 374 | 298 | 376 | 377 |
| TCR W | 380 | 294 | 382 | 384 | 300 | 386 | 387 |
| TCR X | 390 | 294 | 392 | 394 | 298 | 396 | 397 |
| TCR Y | 400 | 294 | 402 | 404 | 298 | 406 | 407 |
| TCR Z | 410 | 294 | 412 | 414 | 300 | 416 | 417 |
| TCR AA | 420 | 294 | 422 | 424 | 298 | 426 | 427 |
| TCR AB | 430 | 294 | 432 | 434 | 300 | 436 | 437 |
| TCR AC | 440 | 294 | 442 | 444 | 300 | 446 | 447 |
| TCR AD | 450 | 294 | 452 | 454 | 298 | 456 | 457 |
| TCR AJ | 481 | 294 | 483 | 485 | 298 | 487 | 488 |

In some of any of the embodiments provided herein, the alpha or gamma chain comprises SEQ ID NO:6, or a sequence that has at least 90% sequence identity thereto, and the beta or delta chain comprises SEQ ID NO:14, or a sequence that has at least 90% sequence identity thereto. In some of any of the embodiments provided herein, the alpha or gamma chain comprises SEQ ID NO:24, or a sequence that has at least 90% sequence identity thereto, and the beta or delta chain comprises SEQ ID NO:30, or a sequence that has at least 90% sequence identity thereto. In some of any of the embodiments provided herein, the alpha or gamma chain comprises SEQ ID NO:40, or a sequence that has at least 90% sequence identity thereto, and the beta or delta chain comprises SEQ ID NO:46, or a sequence that has at least 90% sequence identity thereto. In some of any of the embodiments provided herein, the alpha or gamma chain comprises SEQ ID NO:54, or a sequence that has at least 90% sequence identity thereto, and the beta or delta chain comprises SEQ ID NO:60, or a sequence that has at least 90% sequence identity thereto. In some of any of the embodiments provided herein, the alpha or gamma chain comprises SEQ ID NO:68, or a sequence that has at least 90% sequence identity thereto, and the beta or delta chain comprises SEQ ID NO:71, or a sequence that has at least 90% sequence identity thereto. In some of any of the embodiments provided herein, the alpha or gamma chain comprises SEQ ID NO:81, or a sequence that has at least 90% sequence identity thereto, and the beta or delta chain comprises SEQ ID NO:87, or a sequence that has at least 90% sequence identity thereto. In some of any of the embodiments provided herein, the alpha or gamma chain comprises SEQ ID NO:95, or a sequence that has at least 90% sequence identity thereto, and the beta or delta chain comprises SEQ ID NO:101, or a sequence that has at least 90% sequence identity thereto. In some of any of the embodiments provided herein, the alpha or gamma chain comprises SEQ ID NO: 109, or a sequence that has at least 90% sequence identity thereto, and the beta or delta chain comprises SEQ ID NO:115, or a sequence that has at least 90% sequence identity thereto. In some of any of the embodiments provided herein, the alpha or gamma chain comprises SEQ ID NO:123, or a sequence that has at least 90% sequence identity thereto, and the beta or delta chain comprises SEQ ID NO: 129, or a sequence that has at least 90% sequence identity thereto. In some of any of the embodiments provided herein, the alpha or gamma chain comprises SEQ ID NO:139, or a sequence that has at least 90% sequence identity thereto, and the beta or delta chain comprises SEQ ID NO:145, or a sequence that has at least 90% sequence identity thereto. In some of any of the embodiments provided herein, the alpha or gamma chain comprises SEQ ID NO:155, or a sequence that has at least 90% sequence identity thereto, and the beta or delta chain comprises SEQ ID NO: 161, or a sequence that has at least 90% sequence identity thereto. In some of any of the embodiments provided herein, the alpha or gamma chain comprises SEQ ID NO:169, or a sequence that has at least 90% sequence identity thereto, and the beta or delta chain comprises SEQ ID NO:175, or a sequence that has at least 90% sequence identity thereto. In some of any of the embodiments provided herein, the alpha or gamma chain comprises SEQ ID NO: 183, or a sequence that has at least 90% sequence identity thereto, and the beta or delta chain comprises SEQ ID NO:189, or a sequence that has at least 90% sequence identity thereto. In some of any of the embodiments provided herein, the alpha or gamma chain comprises SEQ ID NO:197, or a sequence that has at least 90% sequence identity thereto, and the beta or delta chain comprises SEQ ID NO:203, or a sequence that has at least 90% sequence identity thereto. In some of any of the embodiments provided herein, the alpha or gamma chain comprises SEQ ID NO:211, or a sequence that has at least 90% sequence identity thereto, and the beta or delta chain comprises SEQ ID NO:217, or a sequence that has at least 90% sequence identity thereto. In some of any of the embodiments provided herein, the alpha or gamma chain comprises SEQ ID NO:227, or a sequence that has at least 90% sequence identity thereto, and the beta or delta chain comprises SEQ ID NO:233, or a sequence that has at least 90% sequence identity thereto. In some of any of the embodiments provided herein, the alpha or gamma chain comprises SEQ ID NO:241, or a sequence that has at least 90% sequence identity thereto, and the beta or delta chain comprises SEQ ID NO:247, or a sequence that has at least 90% sequence identity thereto. In some of any of the embodiments provided herein, the alpha or gamma chain comprises SEQ ID NO:255, or a sequence that has at least 90% sequence identity thereto, and the beta or delta chain comprises SEQ ID NO:261, or a sequence that has at least 90% sequence identity thereto. In some of any of the embodiments provided herein, the alpha or gamma chain comprises SEQ ID NO:271, or a sequence that has at least 90% sequence identity thereto, and the beta or delta chain comprises SEQ ID NO: 161, or a sequence that has at least 90% sequence identity thereto. In some of any of the embodiments provided herein, the alpha or gamma chain comprises SEQ ID NO:281, or a sequence that has at least 90% sequence identity thereto, and the beta or delta chain comprises SEQ ID NO:287, or a sequence that has at least 90% sequence identity thereto. In some of any of the embodiments provided herein, the alpha or gamma chain comprises SEQ ID NO:362, or a sequence that has at least 90% sequence identity thereto, and the beta or delta chain comprises SEQ ID NO:366, or a sequence that has at least 90% sequence identity thereto. In some of any of the embodiments provided herein, the alpha or gamma chain comprises SEQ ID NO:372 or a sequence that has at least 90% sequence identity thereto, and the beta or delta chain comprises SEQ ID NO:376, or a sequence that has at least 90% sequence identity thereto. In some of any of the embodiments provided herein, the alpha or gamma chain comprises SEQ ID NO:382, or a sequence that has at least 90% sequence identity thereto, and the beta or delta chain comprises SEQ ID NO:386, or a sequence that has at least 90% sequence identity thereto. In some of any of the embodiments provided herein, the alpha or gamma chain comprises SEQ ID NO:392, or a sequence that has at least 90% sequence identity thereto, and the beta or delta chain comprises SEQ ID NO:396, or a sequence that has at least 90% sequence identity thereto. In some of any of the embodiments provided herein, the alpha or gamma chain comprises SEQ ID NO:402, or a sequence that has at least 90% sequence identity thereto, and the beta or delta chain comprises SEQ ID NO:406, or a sequence that has at least 90% sequence identity thereto. In some of any of the embodiments provided herein, the alpha or gamma chain comprises SEQ ID NO:412, or a sequence that has at least 90% sequence identity thereto, and the beta or delta chain comprises SEQ ID NO:416, or a sequence that has at least 90% sequence identity thereto. In some of any of the embodiments provided herein, the alpha or gamma chain comprises SEQ ID NO:422, or a sequence that has at least 90% sequence identity thereto, and the beta or delta chain comprises SEQ ID NO:426, or a sequence that has at least 90% sequence identity thereto. In some of any of the embodiments provided herein, the alpha or gamma chain comprises SEQ ID NO:432, or a sequence that has at least 90% sequence identity thereto, and the beta or delta chain comprises SEQ ID NO:436, or a sequence that has at least 90% sequence identity thereto. In some of any of the embodiments provided herein, the alpha or gamma chain comprises SEQ ID NO:442, or a sequence that has at least 90% sequence identity thereto, and the beta or delta chain comprises SEQ ID NO:446, or a sequence that has at least 90% sequence identity thereto. In some of any of the embodiments provided herein, the alpha or gamma chain comprises SEQ ID NO:452, or a sequence that has at least 90% sequence identity thereto, and the beta or delta chain comprises SEQ ID NO:456, or a sequence that has at least 90% sequence identity thereto. In some of any of the embodiments provided herein, the alpha or gamma chain comprises SEQ ID NO:483, or a sequence that has at least 90% sequence identity thereto, and the beta or delta chain comprises SEQ ID NO:487, or a sequence that has at least 90% sequence identity thereto.

In some of any of the embodiments provided herein, the alpha chain comprises SEQ ID NO:6, and the beta chain comprises SEQ ID NO:14. In some of any of the embodiments provided herein, the alpha chain comprises SEQ ID NO:24, and the beta chain comprises SEQ ID NO:30. In some of any of the embodiments provided herein, the alpha chain comprises SEQ ID NO:40, and the beta chain comprises SEQ ID NO:46. In some of any of the embodiments provided herein, the alpha chain comprises SEQ ID NO:54, and the beta chain comprises SEQ ID NO:60. In some of any of the embodiments provided herein, the alpha chain comprises SEQ ID NO:68, and the beta chain comprises SEQ ID NO:71. In some of any of the embodiments provided herein, the alpha chain comprises SEQ ID NO:81, and the beta chain comprises SEQ ID NO:87. In some of any of the embodiments provided herein, the alpha chain comprises SEQ ID NO:95, and the beta chain comprises SEQ ID NO: 101. In some of any of the embodiments provided herein, the alpha chain comprises SEQ ID NO: 109, and the beta chain comprises SEQ ID NO:115. In some of any of the embodiments provided herein, the alpha chain comprises SEQ ID NO: 123, and the beta chain comprises SEQ ID NO: 129. In some of any of the embodiments provided herein, the alpha chain comprises SEQ ID NO:139, and the beta chain comprises SEQ ID NO: 145. In some of any of the embodiments provided herein, the alpha chain comprises SEQ ID NO:155, and the beta chain comprises SEQ ID NO: 161. In some of any of the embodiments provided herein, the alpha chain comprises SEQ ID NO:169, and the beta chain comprises SEQ ID NO:175. In some of any of the embodiments provided herein, the alpha chain comprises SEQ ID NO: 183, and the beta chain comprises SEQ ID NO: 189. In some of any of the embodiments provided herein, the alpha chain comprises SEQ ID NO:197, and the beta chain comprises SEQ ID NO:203. In some of any of the embodiments provided herein, the alpha chain comprises SEQ ID NO:211, and the beta chain comprises SEQ ID NO:217. In some of any of the embodiments provided herein, the alpha chain comprises SEQ ID NO:227, and the beta chain comprises SEQ ID NO:233. In some of any of the embodiments provided herein, the alpha chain comprises SEQ ID NO:241, and the beta chain comprises SEQ ID NO:247. In some of any of the embodiments provided herein, the alpha chain comprises SEQ ID NO:255, and the beta chain comprises SEQ ID NO:261. In some of any of the embodiments provided herein, the alpha chain comprises SEQ ID NO:271, and the beta chain comprises SEQ ID NO: 161. In some of any of the embodiments provided herein, the alpha chain comprises SEQ ID NO:281, and the beta chain comprises SEQ ID NO:287. In some of any of the embodiments provided herein, the alpha or gamma chain comprises SEQ ID NO:362, and the beta or delta chain comprises SEQ ID NO:366. In some of any of the embodiments provided herein, the alpha or gamma chain comprises SEQ ID NO:372, and the beta or delta chain comprises SEQ ID NO:376. In some of any of the embodiments provided herein, the alpha or gamma chain comprises SEQ ID NO:382, and the beta or delta chain comprises SEQ ID NO:386. In some of any of the embodiments provided herein, the alpha or gamma chain comprises SEQ ID NO:392, and the beta or delta chain comprises SEQ ID NO:396. In some of any of the embodiments provided herein, the alpha or gamma chain comprises SEQ ID NO:402, and the beta or delta chain comprises SEQ ID NO:406. In some of any of the embodiments provided herein, the alpha or gamma chain comprises SEQ ID NO:412, and the beta or delta chain comprises SEQ ID NO:416. In some of any of the embodiments provided herein, the alpha or gamma chain comprises SEQ ID NO:422, and the beta or delta chain comprises SEQ ID NO:426. In some of any of the embodiments provided herein, the alpha or gamma chain comprises SEQ ID NO:432, and the beta or delta chain comprises SEQ ID NO:436. In some of any of the embodiments provided herein, the alpha or gamma chain comprises SEQ ID NO:442, and the beta or delta chain comprises SEQ ID NO:446. In some of any of the embodiments provided herein, the alpha or gamma chain comprises SEQ ID NO:452, and the beta or delta chain comprises SEQ ID NO:456. In some of any of the embodiments provided herein, the alpha or gamma chain comprises SEQ ID NO:483, and the beta or delta chain comprises SEQ ID NO:487.

### II. Nucleic Acids Encoding a Tcr

Also provided herein are nucleic acids, such as polynucleotides or nucleic acid molecules, encoding any of the provided TCRs or antigen-binding fragments thereof. The nucleic acids may include those encompassing natural and/or non-naturally occurring nucleotides and bases, e.g., including those with backbone modifications. The terms “nucleic acid molecule,” “nucleic acid,” and “polynucleotide” may be used interchangeably, and refer to a polymer of nucleotides. Such polymers of nucleotides may contain natural and/or non-natural nucleotides, and include, but are not limited to, DNA, RNA, and PNA. “Nucleic acid sequence” refers to the linear sequence of nucleotides that comprise the nucleic acid molecule or polynucleotide.

In some embodiments, a TCR or antigen binding portion thereof provided herein may be a recombinantly produced natural protein or mutated form thereof in which one or more properties, such as a binding characteristic, has been altered. In some aspects, the nucleic acid is synthetic. In some cases, the nucleic acid is or contains cDNA. In some aspects, the polynucleotide can be modified for use in a construct described herein, such as for codon optimization. In some cases, the sequences can be designed to contain terminal restriction site sequences for purposes of cloning into vectors.

In some embodiments, a TCR or antigen-binding portion thereof provided herein can be synthetically generated from knowledge of the sequence of the TCR.

In some embodiments, the polynucleotide contains a nucleic acid sequence encoding an alpha chain and/or a nucleotide sequence encoding a beta chain. In some embodiments, the polynucleotide contains a nucleic acid sequence encoding a gamma chain and/or a nucleotide sequence encoding a delta chain.

In some embodiments, the nucleotide sequence encoding the alpha or gamma chain and/or the nucleotide sequence encoding the beta or delta chain, or any domains, regions or portion thereof, is codon-optimized. Typically, codon optimization involves balancing the percentages of codons selected with the published abundance of human transfer RNAs so that none is overloaded or limiting. This may be necessary in some cases because most amino acids are encoded by more than one codon, and codon usage varies from organism to organism. Differences in codon usage between transfected genes and host cells can have effects on protein expression and immunogenicity of a nucleic acid construct. In general, for codon optimization, codons are chosen to select for those codons that are in balance with human usage frequency. Typically, the redundancy of the codons for amino acids is such that different codons code for one amino acid. In some embodiments, in selecting a codon for replacement, it may be desired that the resulting mutation is a silent mutation such that the codon change does not affect the amino acid sequence. Generally, the last nucleotide of the codon can be changed without affecting the amino acid sequence.

In some of any of the embodiments provided herein, the nucleotide sequence encoding the Vα region comprises SEQ ID NO:7 or a sequence that has at least 90% sequence identity thereto, and the nucleotide sequence encoding the Vβ region comprises SEQ ID NO: 15 or a sequence that has at least 90% sequence identity thereto. In some of any of the embodiments provided herein, the nucleotide sequence encoding the Vα region comprises SEQ ID NO:25 or a sequence that has at least 90% sequence identity thereto, and the nucleotide sequence encoding the Vβ region comprises SEQ ID NO:31 or a sequence that has at least 90% sequence identity thereto. In some of any of the embodiments provided herein, the nucleotide sequence encoding the Vα region comprises SEQ ID NO:41 or a sequence that has at least 90% sequence identity thereto, and the nucleotide sequence encoding the Vβ region comprises SEQ ID NO:47 or a sequence that has at least 90% sequence identity thereto. In some of any of the embodiments provided herein, the nucleotide sequence encoding the Vα region comprises SEQ ID NO:55 or a sequence that has at least 90% sequence identity thereto, and the nucleotide sequence encoding the Vβ region comprises SEQ ID NO:61 or a sequence that has at least 90% sequence identity thereto. In some of any of the embodiments provided herein, the nucleotide sequence encoding the Vα region comprises SEQ ID NO:69 or a sequence that has at least 90% sequence identity thereto, and the nucleotide sequence encoding the Vβ region comprises SEQ ID NO:72 or a sequence that has at least 90% sequence identity thereto. In some of any of the embodiments provided herein, the nucleotide sequence encoding the Vα region comprises SEQ ID NO:82 or a sequence that has at least 90% sequence identity thereto, and the nucleotide sequence encoding the Vβ region comprises SEQ ID NO:88 or a sequence that has at least 90% sequence identity thereto. In some of any of the embodiments provided herein, the nucleotide sequence encoding the Vα region comprises SEQ ID NO:96 or a sequence that has at least 90% sequence identity thereto, and the nucleotide sequence encoding the Vβ region comprises SEQ ID NO:102 or a sequence that has at least 90% sequence identity thereto. In some of any of the embodiments provided herein, the nucleotide sequence encoding the Vα region comprises SEQ ID NO:110 or a sequence that has at least 90% sequence identity thereto, and the nucleotide sequence encoding the Vβ region comprises SEQ ID NO: 116 or a sequence that has at least 90% sequence identity thereto. In some of any of the embodiments provided herein, the nucleotide sequence encoding the Vα region comprises SEQ ID NO:124 or a sequence that has at least 90% sequence identity thereto, and the nucleotide sequence encoding the Vβ region comprises SEQ ID NO:130 or a sequence that has at least 90% sequence identity thereto. In some of any of the embodiments provided herein, the nucleotide sequence encoding the Vα region comprises SEQ ID NO:140 or a sequence that has at least 90% sequence identity thereto, and the nucleotide sequence encoding the Vβ region comprises SEQ ID NO:146 or a sequence that has at least 90% sequence identity thereto. In some of any of the embodiments provided herein, the nucleotide sequence encoding the Vα region comprises SEQ ID NO:156 or a sequence that has at least 90% sequence identity thereto, and the nucleotide sequence encoding the Vβ region comprises SEQ ID NO: 162 or a sequence that has at least 90% sequence identity thereto. In some of any of the embodiments provided herein, the nucleotide sequence encoding the Vα region comprises SEQ ID NO: 170 or a sequence that has at least 90% sequence identity thereto, and the nucleotide sequence encoding the Vβ region comprises SEQ ID NO: 176 or a sequence that has at least 90% sequence identity thereto. In some of any of the embodiments provided herein, the nucleotide sequence encoding the Vα region comprises SEQ ID NO:184 or a sequence that has at least 90% sequence identity thereto, and the nucleotide sequence encoding the Vβ region comprises SEQ ID NO: 190 or a sequence that has at least 90% sequence identity thereto. In some of any of the embodiments provided herein, the nucleotide sequence encoding the Vα region comprises SEQ ID NO:198 or a sequence that has at least 90% sequence identity thereto, and the nucleotide sequence encoding the Vβ region comprises SEQ ID NO:204 or a sequence that has at least 90% sequence identity thereto. In some of any of the embodiments provided herein, the nucleotide sequence encoding the Vα region comprises SEQ ID NO:212 or a sequence that has at least 90% sequence identity thereto, and the nucleotide sequence encoding the Vβ region comprises SEQ ID NO:218 or a sequence that has at least 90% sequence identity thereto. In some of any of the embodiments provided herein, the nucleotide sequence encoding the Vα region comprises SEQ ID NO:228 or a sequence that has at least 90% sequence identity thereto, and the nucleotide sequence encoding the Vβ region comprises SEQ ID NO:234 or a sequence that has at least 90% sequence identity thereto. In some of any of the embodiments provided herein, the nucleotide sequence encoding the Vα region comprises SEQ ID NO:242 or a sequence that has at least 90% sequence identity thereto, and the nucleotide sequence encoding the Vβ region comprises SEQ ID NO:248 or a sequence that has at least 90% sequence identity thereto. In some of any of the embodiments provided herein, the nucleotide sequence encoding the Vα region comprises SEQ ID NO:256 or a sequence that has at least 90% sequence identity thereto, and the nucleotide sequence encoding the Vβ region comprises SEQ ID NO:262 or a sequence that has at least 90% sequence identity thereto. In some of any of the embodiments provided herein, the nucleotide sequence encoding the Vα region comprises SEQ ID NO:272 or a sequence that has at least 90% sequence identity thereto, and the nucleotide sequence encoding the Vβ region comprises SEQ ID NO:274 or a sequence that has at least 90% sequence identity thereto. In some of any of the embodiments provided herein, the nucleotide sequence encoding the Vα region comprises SEQ ID NO:282 or a sequence that has at least 90% sequence identity thereto, and the nucleotide sequence encoding the Vβ region comprises SEQ ID NO:288 or a sequence that has at least 90% sequence identity thereto.

In some of any of the embodiments provided herein, the nucleotide sequence encoding the alpha or gamma chain comprises SEQ ID NO:8, and the nucleotide sequence encoding the beta or delta chain comprises SEQ ID NO:16. In some of any of the embodiments provided herein, the nucleotide sequence encoding the alpha or gamma chain comprises SEQ ID NO:26, and the nucleotide sequence encoding the beta or delta chain comprises SEQ ID NO:32. In some of any of the embodiments provided herein, the nucleotide sequence encoding the alpha or gamma chain comprises SEQ ID NO:42, and the nucleotide sequence encoding the beta or delta chain comprises SEQ ID NO:48. In some of any of the embodiments provided herein, the nucleotide sequence encoding the alpha or gamma chain comprises SEQ ID NO:56, and the nucleotide sequence encoding the beta or delta chain comprises SEQ ID NO:62. In some of any of the embodiments provided herein, the nucleotide sequence encoding the alpha or gamma chain comprises SEQ ID NO:70, and the nucleotide sequence encoding the beta or delta chain comprises SEQ ID NO:73. In some of any of the embodiments provided herein, the nucleotide sequence encoding the alpha or gamma chain comprises SEQ ID NO:83, and the nucleotide sequence encoding the beta or delta chain comprises SEQ ID NO:89. In some of any of the embodiments provided herein, the nucleotide sequence encoding the alpha or gamma chain comprises SEQ ID NO:97, and the nucleotide sequence encoding the beta or delta chain comprises SEQ ID NO:103. In some of any of the embodiments provided herein, the nucleotide sequence encoding the alpha or gamma chain comprises SEQ ID NO: 111, and the nucleotide sequence encoding the beta or delta chain comprises SEQ ID NO: 117. In some of any of the embodiments provided herein, the nucleotide sequence encoding the alpha or gamma chain comprises SEQ ID NO:125, and the nucleotide sequence encoding the beta or delta chain comprises SEQ ID NO:131. In some of any of the embodiments provided herein, the nucleotide sequence encoding the alpha or gamma chain comprises SEQ ID NO:141, and the nucleotide sequence encoding the beta or delta chain comprises SEQ ID NO:147. In some of any of the embodiments provided herein, the nucleotide sequence encoding the alpha or gamma chain comprises SEQ ID NO:157, and the nucleotide sequence encoding the beta or delta chain comprises SEQ ID NO: 163. In some of any of the embodiments provided herein, the nucleotide sequence encoding the alpha or gamma chain comprises SEQ ID NO:171, and the nucleotide sequence encoding the beta or delta chain comprises SEQ ID NO: 177. In some of any of the embodiments provided herein, the nucleotide sequence encoding the alpha or gamma chain comprises SEQ ID NO:185, and the nucleotide sequence encoding the beta or delta chain comprises SEQ ID NO:191. In some of any of the embodiments provided herein, the nucleotide sequence encoding the alpha or gamma chain comprises SEQ ID NO:199, and the nucleotide sequence encoding the beta or delta chain comprises SEQ ID NO:205. In some of any of the embodiments provided herein, the nucleotide sequence encoding the alpha or gamma chain comprises SEQ ID NO:213, and the nucleotide sequence encoding the beta or delta chain comprises SEQ ID NO:219. In some of any of the embodiments provided herein, the nucleotide sequence encoding the alpha or gamma chain comprises SEQ ID NO:229, and the nucleotide sequence encoding the beta or delta chain comprises SEQ ID NO:235. In some of any of the embodiments provided herein, the nucleotide sequence encoding the alpha or gamma chain comprises SEQ ID NO:243, and the nucleotide sequence encoding the beta or delta chain comprises SEQ ID NO:249. In some of any of the embodiments provided herein, the nucleotide sequence encoding the alpha or gamma chain comprises SEQ ID NO:257, and the nucleotide sequence encoding the beta or delta chain comprises SEQ ID NO:263. In some of any of the embodiments provided herein, the nucleotide sequence encoding the alpha or gamma chain comprises SEQ ID NO:273, and the nucleotide sequence encoding the beta or delta chain comprises SEQ ID NO:275. In some of any of the embodiments provided herein, the nucleotide sequence encoding the alpha or gamma chain comprises SEQ ID NO:283, and the nucleotide sequence encoding the beta or delta chain comprises SEQ ID NO:289.

In some of any of the embodiments provided herein, the nucleotide sequence encoding the Vα region comprises SEQ ID NO:301, and the nucleotide sequence encoding the Vβ region comprises SEQ ID NO:321. In some of any of the embodiments provided herein, the nucleotide sequence encoding the Vα region comprises SEQ ID NO:302, and the nucleotide sequence encoding the Vβ region comprises SEQ ID NO:322. In some of any of the embodiments provided herein, the nucleotide sequence encoding the Vα region comprises SEQ ID NO:303, and the nucleotide sequence encoding the Vβ region comprises SEQ ID NO:323. In some of any of the embodiments provided herein, the nucleotide sequence encoding the Vα region comprises SEQ ID NO:304, and the nucleotide sequence encoding the Vβ region comprises SEQ ID NO:324. In some of any of the embodiments provided herein, the nucleotide sequence encoding the Vα region comprises SEQ ID NO:305, and the nucleotide sequence encoding the Vβ region comprises SEQ ID NO:325. In some of any of the embodiments provided herein, the nucleotide sequence encoding the Vα region comprises SEQ ID NO:306, and the nucleotide sequence encoding the Vβ region comprises SEQ ID NO:326. In some of any of the embodiments provided herein, the nucleotide sequence encoding the Vα region comprises SEQ ID NO:307, and the nucleotide sequence encoding the Vβ region comprises SEQ ID NO:327. In some of any of the embodiments provided herein, the nucleotide sequence encoding the Vα region comprises SEQ ID NO:308, and the nucleotide sequence encoding the Vβ region comprises SEQ ID NO:328. In some of any of the embodiments provided herein, the nucleotide sequence encoding the Vα region comprises SEQ ID NO:309, and the nucleotide sequence encoding the Vβ region comprises SEQ ID NO:329. In some of any of the embodiments provided herein, the nucleotide sequence encoding the Vα region comprises SEQ ID NO:310, and the nucleotide sequence encoding the Vβ region comprises SEQ ID NO:330. In some of any of the embodiments provided herein, the nucleotide sequence encoding the Vα region comprises SEQ ID NO:311, and the nucleotide sequence encoding the Vβ region comprises SEQ ID NO:331. In some of any of the embodiments provided herein, the nucleotide sequence encoding the Vα region comprises SEQ ID NO:312, and the nucleotide sequence encoding the Vβ region comprises SEQ ID NO:332. In some of any of the embodiments provided herein, the nucleotide sequence encoding the Vα region comprises SEQ ID NO:313, and the nucleotide sequence encoding the Vβ region comprises SEQ ID NO:333. In some of any of the embodiments provided herein, the nucleotide sequence encoding the Vα region comprises SEQ ID NO:314, and the nucleotide sequence encoding the Vβ region comprises SEQ ID NO:334. In some of any of the embodiments provided herein, the nucleotide sequence encoding the Vα region comprises SEQ ID NO:315, and the nucleotide sequence encoding the Vβ region comprises SEQ ID NO:335. In some of any of the embodiments provided herein, the nucleotide sequence encoding the Vα region comprises SEQ ID NO:316, and the nucleotide sequence encoding the Vβ region comprises SEQ ID NO:336. In some of any of the embodiments provided herein, the nucleotide sequence encoding the Vα region comprises SEQ ID NO:317, and the nucleotide sequence encoding the Vβ region comprises SEQ ID NO:337. In some of any of the embodiments provided herein, the nucleotide sequence encoding the Vα region comprises SEQ ID NO:318, and the nucleotide sequence encoding the Vβ region comprises SEQ ID NO:338. In some of any of the embodiments provided herein, the nucleotide sequence encoding the Vα region comprises SEQ ID NO:319, and the nucleotide sequence encoding the Vβ region comprises SEQ ID NO:339. In some of any of the embodiments provided herein, the nucleotide sequence encoding the Vα region comprises SEQ ID NO:320, and the nucleotide sequence encoding the Vβ region comprises SEQ ID NO:340.

In some embodiments, the nucleic acid sequence encoding the alpha or gamma chain comprises one of the following: SEQ ID NO: 8, 26, 42, 56, 70, 83, 97, 111, 125, 141, 157, 171, 185, 199, 213, 229, 243, 257, 273, or 283, a degenerate sequence thereof or a sequence having at least 90%, 91%, 92%, 93%, 94%, 95%, 96%, 97%, 98%, 99% or more sequence identity thereto. In some aspects, the nucleotide sequence encoding the beta or delta chain comprises one of the following: SEQ ID NO: 16, 32, 48, 62, 73, 89, 103, 117, 131, 147, 163, 177, 191, 205, 219, 235, 249, 263, 275, or 289, a degenerate sequence thereof or a sequence having at least 90%, 91%, 92%, 93%, 94%, 95%, 96%, 97%, 98%, 99% or more sequence identity thereto.

In some of any of the embodiments provided herein, the nucleotide sequence encoding the alpha chain comprises SEQ ID NO:8, and the nucleotide sequence encoding the beta chain comprises SEQ ID NO:16. In some of any of the embodiments provided herein, the nucleotide sequence encoding the alpha chain comprises SEQ ID NO:26, and the nucleotide sequence encoding the beta chain comprises SEQ ID NO:32. In some of any of the embodiments provided herein, the nucleotide sequence encoding the alpha chain comprises SEQ ID NO:42, and the nucleotide sequence encoding the beta chain comprises SEQ ID NO:48. In some of any of the embodiments provided herein, the nucleotide sequence encoding the alpha chain comprises SEQ ID NO:56, and the nucleotide sequence encoding the beta chain comprises SEQ ID NO:62. In some of any of the embodiments provided herein, the nucleotide sequence encoding the alpha chain comprises SEQ ID NO:70, and the nucleotide sequence encoding the beta chain comprises SEQ ID NO:73. In some of any of the embodiments provided herein, the nucleotide sequence encoding the alpha chain comprises SEQ ID NO:83, and the nucleotide sequence encoding the beta chain comprises SEQ ID NO:89. In some of any of the embodiments provided herein, the nucleotide sequence encoding the alpha chain comprises SEQ ID NO:97, and the nucleotide sequence encoding the beta chain comprises SEQ ID NO: 103. In some of any of the embodiments provided herein, the nucleotide sequence encoding the alpha chain comprises SEQ ID NO:111, and the nucleotide sequence encoding the beta chain comprises SEQ ID NO:117. In some of any of the embodiments provided herein, the nucleotide sequence encoding the alpha chain comprises SEQ ID NO:125, and the nucleotide sequence encoding the beta chain comprises SEQ ID NO: 131. In some of any of the embodiments provided herein, the nucleotide sequence encoding the alpha chain comprises SEQ ID NO: 141, and the nucleotide sequence encoding the beta chain comprises SEQ ID NO: 147. In some of any of the embodiments provided herein, the nucleotide sequence encoding the alpha chain comprises SEQ ID NO:157, and the nucleotide sequence encoding the beta chain comprises SEQ ID NO:163. In some of any of the embodiments provided herein, the nucleotide sequence encoding the alpha chain comprises SEQ ID NO:171, and the nucleotide sequence encoding the beta chain comprises SEQ ID NO: 177. In some of any of the embodiments provided herein, the nucleotide sequence encoding the alpha chain comprises SEQ ID NO:185, and the nucleotide sequence encoding the beta chain comprises SEQ ID NO: 191. In some of any of the embodiments provided herein, the nucleotide sequence encoding the alpha chain comprises SEQ ID NO:199, and the nucleotide sequence encoding the beta chain comprises SEQ ID NO:205. In some of any of the embodiments provided herein, the nucleotide sequence encoding the alpha chain comprises SEQ ID NO:213, and the nucleotide sequence encoding the beta chain comprises SEQ ID NO:219. In some of any of the embodiments provided herein, the nucleotide sequence encoding the alpha chain comprises SEQ ID NO:229, and the nucleotide sequence encoding the beta chain comprises SEQ ID NO:235. In some of any of the embodiments provided herein, the nucleotide sequence encoding the alpha chain comprises SEQ ID NO:243, and the nucleotide sequence encoding the beta chain comprises SEQ ID NO:249. In some of any of the embodiments provided herein, the nucleotide sequence encoding the alpha chain comprises SEQ ID NO:257, and the nucleotide sequence encoding the beta chain comprises SEQ ID NO:263. In some of any of the embodiments provided herein, the nucleotide sequence encoding the alpha chain comprises SEQ ID NO:273, and the nucleotide sequence encoding the beta chain comprises SEQ ID NO:275. In some of any of the embodiments provided herein, the nucleotide sequence encoding the alpha chain comprises SEQ ID NO:283, and the nucleotide sequence encoding the beta chain comprises SEQ ID NO:289. Also among the provided nucleic acid(s) or polynucleotides provided herein are those containing sequences at least at or about 90%, 91%, 92%, 93%, 94%, 95%, 96%, 97%, 98%, or 99% identical to such sequences. Also among the provided embodiments are one or more chains (e.g., alpha or gamma chain and/or beta or delta chain) of a TCR or a binding fragment thereof encoded by any of such polynucleotides.

In some embodiments, the alpha or gamma chain and/or beta or delta chain of the TCR is encoded by a sequence of nucleotides comprising a signal peptide (also called a leader sequence). Non-limiting examples of such a signal peptide are signal peptides that have or comprise the sequence of amino acids set forth in any of SEQ ID NOs: 341-350.

In some embodiments, the nucleic acid encoding the alpha or gamma chain and the nucleic acid encoding the beta or delta chain can be connected via a linker, such as any described elsewhere herein.

In some embodiments, the nucleic acid encoding the alpha or gamma chain and the nucleic acid encoding the beta or delta chain can be connected via a cleavable linker sequence or a peptide that causes ribosome skipping (e.g. T2A or P2A), such as any described elsewhere herein.

Also provided herein are vectors or constructs containing such nucleotide sequences. In some embodiments, the vectors or constructs contain one or more promoters operatively linked to the nucleotide encoding the alpha or gamma chain and/or the beta or delta chain. In some embodiments, the promoter is operatively linked to one or more than one nucleotide sequence.

In some embodiments, the vector or construct can contain a single promoter that drives the expression of one or more nucleotide sequences. In some embodiments, such promoters can be multicistronic (e.g., bicistronic or tricistronic, see e.g., U.S. Pat. No. 6,060,273). For example, in some embodiments, transcription units can be engineered as a bicistronic unit containing an IRES (internal ribosome entry site), which allows coexpression of gene products (e.g. encoding an alpha or gamma chain and/or a beta or delta chain of a TCR) by a message from a single promoter. Alternatively, in some cases, a single promoter may direct expression of an RNA that contains, in a single open reading frame (ORF), two or three genes (e.g. encoding an alpha or gamma chain and/or a beta or delta chain of a TCR) separated from one another by sequences encoding a self-cleavage peptide (e.g., P2A) or a protease recognition site (e.g., furin). The ORF thus encodes a single polyprotein, which, either during (in the case of 2A e.g., P2A) or after translation, is cleaved into the individual proteins. In some cases, the peptide, such as P2A, can cause the ribosome to skip (ribosome skipping) synthesis of a peptide bond at the C-terminus of a 2A element, leading to separation between the end of the 2A sequence and the next peptide downstream (see, for example, de Felipe. Genetic Vaccines and Ther. 2:13 (2004) and deFelipe et al. Traffic 5:616-626 (2004)). Examples of 2A cleavage peptides, including those that can induce ribosome skipping, are Thosea asigna virus (T2A), porcine teschovirus-1 (P2A, e.g., SEQ ID NO: 352), equine rhinitis A virus (E2A), and 2A sequences from the foot-and-mouth disease virus (F2A) as described in U.S. Patent Publication No. 2007/0116690. In some such instances, the peptide that causes ribosome skipping is a P2A peptide and/or contains the sequence of amino acids set forth in SEQ ID NO:352.

In some embodiments, the nucleic acid sequence encoding the alpha or gamma chain and the nucleotide sequence encoding the beta or delta chain are present in any order, separated by the nucleotide sequence encoding a peptide sequence that causes ribosome skipping. For example, in some embodiments, the nucleotide sequence comprises a nucleic acid sequence encoding a beta or delta chain, a nucleic acid sequence encoding a peptide sequence that causes ribosome skipping, e.g., a P2A sequence as described herein, and a nucleic acid sequence that encodes an alpha or gamma chain, in that order. In other embodiments, the nucleotide sequence contains a nucleic acid sequence that encodes an alpha or gamma chain, a nucleic acid sequence that encodes a peptide sequence that causes ribosome skipping, e.g., a P2A sequence as described herein, and a nucleic acid sequence that encodes a beta or delta chain, in that order.

In some embodiments, the nucleotide sequence encoding an alpha or gamma chain and/or a beta or delta chain of a TCR comprises a nucleic acid sequence corresponding to a SEQ ID NO: set forth in Table 3. Also among the provided nucleotide sequences encoding a TCR are those containing sequences at least at or about 90%, 91%, 92%, 93%, 94%, 95%, 96%, 97%, 98%, or 99% identical to such sequences. Also provided are any of the mature TCR alpha or gamma chains encoded by any of the sequences set forth in Table 3, such as in each row therein. Also provided are any of the mature TCR beta or delta chains encoded by any of the sequences set forth in Table 3, such as in each row therein. Also provided are any of the mature TCR alpha and beta chains, or mature gamma and delta chains, encoded by any of the sequences set forth in Table 3, such as in each row therein. In some aspects, the nucleotide sequences contain sequences encoding a signal sequence, and the encoded exemplary TCRs, when expressed as a mature protein, comprise the mature Vα and/or mature Vβ region, or the mature Vγ and/or Vδ mature region, for example, without the signal sequence (e.g., from cleavage of the signal sequence) when fully processed and expressed.

In some embodiments, the nucleotide sequence encodes a polypeptide containing an amino acid sequence set forth in Table 3, such as in each row therein, or a sequence having at least 90%, 91%, 92%, 93%, 94%, 95%, 96%, 97%, 98%, or 99% sequence identity thereto. In some embodiments, the nucleotide sequence encodes a mature polypeptide set forth herein, for example, in Table 3, such as in each row therein, or a sequence having at least 90%, 91%, 92%, 93%, 94%, 95%, 96%, 97%, 98%, or 99% sequence identity thereto.

TABLE 3
SEQ ID NOs of Nucleotide Sequences of HA-1 Specific TCRs

| | Alpha | | | | Beta | | | | |
|---|---|---|---|---|---|---|---|---|---|
| | | Vα | | | | Vβ | | | |
| | | (nt) | | Vα + | | (nt) | | Vβ + | |
| | Vα | codon | Cα | Cα | Vβ | codon | Cβ | Cβ | alpha-P2A- |
| TCR | (nt) | opt. | (nt) | (nt) | (nt) | opt. | (nt) | (nt) | beta |
| TCR A | 7 | 301 | 293 | 8 | 15 | 321 | 299 | 16 | 17 |
| TCR B | 25 | 302 | 293 | 26 | 31 | 322 | 297 | 32 | 33 |
| TCR C | 41 | 303 | 293 | 42 | 47 | 323 | 297 | 48 | 49 |
| TCR D | 55 | 304 | 293 | 56 | 61 | 324 | 299 | 62 | 63 |
| TCR E | 69 | 305 | 293 | 70 | 72 | 325 | 297 | 73 | 74 |
| TCR F | 82 | 306 | 295 | 83 | 88 | 326 | 299 | 89 | 90 |
| TCR G | 96 | 307 | 295 | 97 | 102 | 327 | 299 | 103 | 104 |
| TCR H | 110 | 308 | 293 | 111 | 116 | 328 | 299 | 117 | 118 |
| TCR I | 124 | 309 | 293 | 125 | 130 | 329 | 299 | 131 | 132 |
| TCR J | 140 | 310 | 293 | 141 | 146 | 330 | 299 | 147 | 148 |
| TCR K | 156 | 311 | 293 | 157 | 162 | 331 | 299 | 163 | 164 |
| TCR L | 170 | 312 | 293 | 171 | 176 | 332 | 297 | 177 | 178 |
| TCR M | 184 | 313 | 293 | 185 | 190 | 333 | 299 | 191 | 192 |
| TCR N | 198 | 314 | 295 | 199 | 204 | 334 | 299 | 205 | 206 |
| TCR O | 212 | 315 | 293 | 213 | 218 | 335 | 299 | 219 | 220 |
| TCR P | 228 | 316 | 293 | 229 | 234 | 336 | 299 | 235 | 236 |
| TCR Q | 242 | 317 | 295 | 243 | 248 | 337 | 297 | 249 | 250 |
| TCR R | 256 | 318 | 293 | 257 | 262 | 338 | 297 | 263 | 264 |
| TCR S | 272 | 319 | 295 | 273 | 274 | 339 | 299 | 275 | 276 |
| TCR T | 282 | 320 | 293 | 283 | 288 | 340 | 299 | 289 | 290 |
| TCR U | | | | | | | | | 368 |
| TCR V | | | | | | | | | 378 |
| TCR W | | | | | | | | | 388 |
| TCR X | | | | | | | | | 398 |
| TCR Y | | | | | | | | | 408 |
| TCR Z | | | | | | | | | 418 |
| TCR AA | | | | | | | | | 428 |
| TCR AB | | | | | | | | | 438 |
| TCR AC | | | | | | | | | 448 |
| TCR AD | | | | | | | | | 458 |
| TCR AJ | | | | | | | | | 489 |

The nucleic acid may encode an amino acid sequence comprising the Vα region of the TCR. In some cases, the nucleic acid encodes an amino acid sequence comprising the Vβ region of the TCR. In a further embodiment, one or more vectors (e.g., expression vectors) comprising such nucleic acid are provided.

Also provided herein are vectors, such as those containing any of the nucleic acids described herein. In some embodiments, nucleic acid or nucleic acids encoding one or both chains of a TCR, are cloned or assembled into a suitable expression vector or vectors. The expression vector can be any suitable recombinant expression vector, and can be used to transform or transfect any suitable host. Suitable vectors include those designed for propagation and expansion or for expression or both, such as plasmids and viruses. In some embodiments, the vector is an expression vector.

### III. Methods for Isolating, Assessing and Identifying T Cell Receptors

In some aspects, provided herein are methods for isolating a plurality of nucleic acid sequences encoding the provided TCRs specific for a minor histocompatibility antigen. In some aspects, the provided HA-1 specific TCRs are identified based on the methods described herein. In some aspects, the methods also include isolating nucleic acid sequences, assembling the nucleic acid sequences into vectors, assessing the expression and/or activity of the TCRs, and screening and identifying particular TCRs of interest, in some cases, using a high-throughput method.

The methods described herein also relate to determining the binding activity and functional capacity of candidate TCRs.

A. Donor Criteria and Methods for Isolating miHA-Specific T Cell Candidates

Alloreactive T cells have been isolated from patients who have undergone an alloSCT. However, such patients frequently have few circulating T cells, undergo immunosuppressive treatments, and may have other alloSCT-related illnesses that could compromise aspects of donor candidacy, including timely blood collection. To improve elements of the blood collection process and the quality of donor samples, an alternative donor source was considered. Parous women, including multiparous women, may have been naturally immunized to paternal miHA during pregnancy and at the time of delivery. In some cases, miHA-reactive T cells are expanded as a result of pregnancy. See Lee et al. (2019) Biol Blood Marrow Transpl. 25(4):625-638. In some embodiments, the T cells have been obtained from naïve male PBMCs.

In some aspects, whole exome sequencing is used to type parous volunteers for their HLA repertoire and for polymorphisms that encode the HA-1 miHA or the nonimmunogenic variant. For those subjects with an appropriate HLA type, which lacked HA-1, the child/children or father of the progeny are then typed to determine whether they expressed the desired HA-1. If so, it is possible that the mother is immunized during pregnancy/childbirth. Such suitable parous women are recruited for blood collection. Anti-HA-1 reactive T cells from a parous woman are identified on the basis of their binding an HLA-multimer folded with the HA-1 peptide. These cells are single cell sorted and their TCRs are screened for anti-HA-1 reactivity.

### B. High-Throughput Isolation, Amplification and Assembly of Nucleic Acid Sequences Encoding TCRs

In some aspects, nucleic acid molecules encoding a TCR can be obtained or identified from a variety of sources. In some aspects, TCRs can be obtained or identified using a high-throughput TCR isolation and screening method. Examples of such methods that can be used include those described in, for example, WO2018/102473, which is incorporated by reference in its entirety. In some aspects, the high-throughput TCR isolation and screening methods involve the amplification of nucleic acids encoding TCR alpha and/or beta chains, or TCR gamma and/or delta chains, from a plurality of different cells, such as T cells, isolated from a donor. Also provided herein are such methods related to isolating or screening a plurality of different TCRs to obtain TCRs that are specific for a hematopoietically restricted minor histocompatibility antigen, such as a HA-1 peptide.

In some embodiments, nucleic acid molecules encoding a TCR can be obtained from a variety of sources, such as by polymerase chain reaction (PCR) amplification of encoding nucleic acids within or isolated from a given cell or cells. In some embodiments, a TCR is obtained from a biological source, such as from cells such as from T cells (e.g. cytotoxic T cells), T cell hybridomas, or other publicly available sources. In some embodiments, a TCR may be derived from one of various animal species, such as a human, mouse, rat, or other mammal. In some embodiments, the T cells can be obtained from in vivo isolated cells, such as from normal (or healthy) subjects or diseased subjects, including T cells present in peripheral blood mononuclear cells (PBMCs) or tumor-infiltrating lymphocytes (TILs). In some embodiments, the T cells can be a cultured T cell hybridoma or clone. For example, in some embodiments, to generate a vector encoding a TCR, the α and β chains can be PCR amplified from total cDNA isolated from a T cell clone expressing the TCR of interest and cloned into an expression vector. In some embodiments, the α and β chains can be synthetically generated. In some embodiments, the α and β chains are cloned into the same vector.

As described herein, the methods and materials provided herein can allow users to capture successfully most, if not all, functional TCRs from a sorted T cell population. For example, an amplification (e.g., nested amplification procedure such as a nested PCR) procedure can include using primer collections designed to amplify every known functional V segment of the two variable chains of a particular TCR (e.g., any of the known functional V segments of the α variable and β variable chains of a particular αβ TCR or any of the known functional V segments of the γ variable and 8 variable chains of a particular γδ TCR) of a mammal (e.g., a human). For humans, an amplification procedure can include a primer collection designed to amplify all 45 V segments of the α chain currently known to be functional and all 48 V segments of the β chain currently known to be functional. When referring to TCR V segments of the α chain herein, the shorthand abbreviation TRAV can be used. Likewise, when referring to TCR V segments of the β chain herein, the shorthand abbreviation TRBV can be used. The same is true for TCR V segments of the γ and δ chains, which can be referred to as TRGV and TRDV, respectively.

In some aspects, this document provides methods and materials involved in cloning functional TCRs from single T cells. For example, in some aspects, this document provides methods and materials for obtaining nucleic acid encoding a TCR from a single T cell and arranging that nucleic acid to form nucleic acid vectors successfully designed to express a TCR (e.g., a fully intact TCR such as a fully intact TCR having the variable chain combination as present in that single T cell), kits for obtaining nucleic acid encoding a TCR from a single T cell and arranging that nucleic acid to form nucleic acid vectors successfully designed to express a TCR (e.g., a fully intact TCR such as a fully intact TCR having the variable chain combination as present in that single T cell), and methods for making such kits. A cloned αβ TCR having the variable chain combination as present in a single T cell used to clone that TCR can include the VJ α segment combination as present in that single T cell, the VDJ β segment combination as present in that single T cell, the nucleotide sequence of the entire α variable region as present in that single T cell, and the nucleotide sequence of the entire β variable region as present in that single T cell. Likewise, a cloned γδ TCR having the variable chain combination as present in a single T cell used to clone that TCR can include the VJ γ segment combination as present in that single T cell, the VDJ δ segment combination as present in that single T cell, the nucleotide sequence of the entire γ variable region as present in that single T cell, and the nucleotide sequence of the entire δ variable region as present in that single T cell.

In some aspects, this document provides collections of nucleic acid primers are designed to amplify the entire coding sequence of both variable regions (e.g., the α variable region and β variable region, or the γ variable region and 8 variable region) for each expressed V segment (e.g., each expressed α V segment and β V segment, or each expressed γ V segment and δ V segment) for functional αβ or γδ TCRs of a particular mammalian species (e.g., a mouse or a human), methods for using such collections of nucleic acid primers to clone functional TCRs from single T cells, and kits containing such collections of nucleic acid primers to clone functional TCRs from single T cells.

In some aspects, the methods and materials provided herein can allow one to perform highly multiplexed reactions to clone many different TCRs (e.g., hundreds to thousands or more different TCRs) directly from single T cells quickly (e.g., simultaneously in some cases) and in a manner that misses few, if any, α/β variable chain combinations (or γ/δ variable chain combinations). For example, the methods and materials provided herein can be performed to clone many different αβ TCRs (e.g., hundreds to thousands or more different αβ TCRs) directly from single αβ T cells in a manner that misses less than 10 percent (e.g., less than 9 percent, less than 8 percent, less than 7 percent, less than 6 percent, less than 5 percent, less than 4 percent, less than 3 percent, less than 2 percent, or less than 1 percent) of the α variable chains and less than 10 percent (e.g., less than 9 percent, less than 8 percent, less than 7 percent, less than 6 percent, less than 5 percent, less than 4 percent, less than 3 percent, less than 2 percent, or less than 1 percent) of the β variable chains possible for α/β variable chain combinations of a species (e.g., mice or human species). Likewise, the methods and materials provided herein can be performed to clone many different γδ TCRs (e.g., hundreds to thousands or more different γδ TCRs) directly from single γδ T cells in a manner that misses less than 10 percent (e.g., less than 9 percent, less than 8 percent, less than 7 percent, less than 6 percent, less than 5 percent, less than 4 percent, less than 3 percent, less than 2 percent, or less than 1 percent) of the γ variable chains and less than 10 percent (e.g., less than 9 percent, less than 8 percent, less than 7 percent, less than 6 percent, less than 5 percent, less than 4 percent, less than 3 percent, less than 2 percent, or less than 1 percent) of the δ variable chains possible for γ/δ variable chain combinations of a species (e.g., mice or human species). In some cases, the methods and materials provided herein can include (a) obtaining a sample of T cells, (b) sorting those T cells into isolated locations (e.g., wells) such that most, if not all, isolated locations (e.g., each well) contain a single T cell, (c) lysing (e.g., simultaneously lysing) the single T cells located in separate isolated locations (e.g., separate wells) to release the RNA of each single T cell, (d) performing (e.g., simultaneously performing) reverse transcription using the released RNA as template, appropriate primers for cDNA synthesis from RNA, and a reverse transcriptase enzyme to produce cDNA within each isolated location (e.g., each well); that cDNA representing the RNA expressed by the single T cell that was located in that isolated location (e.g., well), (e) performing (e.g., simultaneously performing), for each isolated location, a first round amplification reaction (e.g., a first round polymerase chain reaction (PCR)) of an amplification procedure (e.g., such as a nested amplification procedure such as a nested PCR) using the produced cDNA as template, a first round primer collection (e.g., a first round PCR primer collection), and a polymerase (e.g., Taq polymerase) to produce at least an amplification product containing a nucleic acid sequence of the α variable chain (or γ variable chain) of the TCR of the single T cell of that isolated location and an amplification product containing a nucleic acid sequence of the β variable chain (or δ variable chain) of the TCR of that same single T cell of that same isolated location, (f) performing (e.g., simultaneously performing), for each isolated location, a second round amplification reaction (e.g., a second round PCR) of a nested amplification procedure (e.g., a nested PCR procedure) using the amplification products of the first round amplification reaction as template, a second round primer collection (e.g., a second round PCR primer collection), and a polymerase (e.g., Taq polymerase) to produce at least a first amplification product containing a nucleic acid sequence of the α variable chain (or γ variable chain) of the TCR of the single T cell of that isolated location and a second amplification product containing a nucleic acid sequence of the β variable chain (or δ variable chain) of the TCR of that same single T cell of that same isolated location, and (g) cloning, for each isolated location, the first and second amplification products into an expression vector designed to express a functional TCR having the α/β or γ/δ variable chain combination (or a portion thereof such as the V segments of the α/β or γ/δ variable chain combination) as was present in the single T cell used to generate the amplification products.

The resulting expression vectors can be introduced into cells such that those cells express the cloned TCRs. Such cells and/or the TCRs they express from the introduced expression vectors can be screened to identify TCRs with desired capabilities. For example, cells expressing cloned TCRs that recognize particular antigens (e.g., peptides derived from tumor polypeptides) can be identified, and those cells, the TCR expression vectors they contain, or the cloned TCR constructs can be used for further analysis or for therapeutic applications.

In some cases, expression of cloned TCRs on the surface and expression of functional TCRs can be assessed by introducing the expression vectors into TCR-negative reporter cells designed to express a measurable marker signal or marker polypeptide once the signaling apparatus of a functional TCR is engaged. In these cases, an antibody designed to non-specifically activate TCRs (e.g., an anti-CD3 antibody) can be used to screen for functional TCRs. In some cases, the cloned TCRs can be screened for antigen specificity. For example, reporter cells expressing cloned TCRs can be screened for the recognition of particular antigens (e.g., peptides derived from tumor polypeptides). In some cases, primary T cells (e.g., human primary T cells) can be transfected with expression vectors and screened for antigen specificity via T cell proliferation assays.

The methods and materials provided herein can allow clinicians, medical professionals, laboratory personnel, and researchers to use a collection of T cells having different TCRs to generate collections of expression vectors that express functional versions of those different TCRs that have the same variable chain combinations or portions thereof (e.g., the same α/β variable chain combination or the same γ/δ variable chain combination) as present in original T cells used to generate the collection. Such collections of expression vectors can be obtained quickly, efficiently, inexpensively, and effectively. For example, in some cases, using the methods and materials provided herein, a collection of expression vectors that express functional versions of many different TCRs with authentic variable chain combinations as found in T cells obtained from a mammal (e.g., a human) can be generated within less than 12 days (e.g., from 4 to 11 days, from 5 to 11 days, from 6 to 11 days, from 7 to 11 days, from 8 to 11 days, from 4 to 10 days, from 5 to 10 days, from 6 to 10 days, from 7 to 10 days, from 8 to 10 days, from 4 to 9 days, from 5 to 9 days, from 6 to 9 days, from 7 to 9 days, from 4 to 8 days, from 5 to 8 days, from 6 to 8 days, or from 7 to 8 days), using less than 12 steps (e.g., from 5 to 11 steps, from 6 to 11 steps, from 7 to 11 steps, from 8 to 11 steps, from 5 to 10 steps, from 6 to 10 steps, from 7 to 10 steps, from 8 to 10 steps, from 5 to 9 steps, from 6 to 9 steps, from 7 to 9 steps, or from 8 to 9 steps), for less than about 10 dollars per TCR, and with greater than about 80 percent (e.g., greater than about 85, 90, or 95 percent) effectiveness (based on sorting a single T cell into each of 384 wells of 384-well plate). In some cases, the methods and materials provided herein can be performed without performing nucleic acid sequencing, without performing restriction endonuclease cleavage steps, without performing other steps or techniques as described herein, and/or without using particular reagents or materials as described herein.

The methods and materials provided herein also can allow users to capture successfully most, if not all, functional TCRs from a sorted T cell population. For example, in some cases, the methods and materials provided herein can include a nested amplification procedure (e.g., a nested PCR procedure) that includes primer collections designed to amplify every known functional V segment of the two variable chains of a particular TCR (e.g., any of the known functional V segments of the α variable and B variable chains of a particular αβ TCR or any of the known functional V segments of the γ variable and δ variable chains of a particular γδ TCR) of a mammal (e.g., a human). Having the ability to clone most, if not all, functional TCRs from a sorted T cell population can allow users to identify particular TCRs, including rare TCRs, that might otherwise be missed. It is these rare TCRs that might be missed that could provide a rich source of new cloned TCRs for effective therapies such as cancer therapies involving the delivery of effective T cells.

In some cases, the methods and materials provided herein can allow users to obtain additional information about the single T cells from which functional TCR clones are generated. In some cases, the flow cytometry techniques used for single cell sorting described herein can be used to distinguish activated and experienced cells from naïve T cells by staining those cells for activation markers. When applying the methods and materials provided herein in methods for treating a particular disease (e.g., cancer), T cells can be isolated from a patient that have already been activated and expanded within that patient. Once these T cells are isolated, and cDNA is generated from single cell RNA, an additional level of selection can be applied. For example, in addition to using cDNA produced from the RNA of a single T cell to amplify and clone the variable chains (or portions thereof) of that T cell's TCR, that cDNA also can be used to assess RNA expression and/or RNA expression levels within that T cell.

In the case of CD8″ T cells, TCRs associated with polyfunctional (e.g., multi-cytokine producers) effector cells or TCRs associated with quiescent or exhausted long-lived memory cells can be identified by examining the relative mRNA levels for expression of transcription factors such as Eomesodermin and T-bet (McLane et al., J. Immunol., 190(7):3207-3215 (2013); and Buggert et al., PLOS Pathog., 10(7):e1004251 (2014)).

In some cases, T cells can be stimulated (e.g., in vitro stimulated) prior to sorting, and then RNA expression can be assessed (via, e.g., qPCR) to determine which T cells responded to the stimulation. Any appropriate type of stimulation can be used including, without limitation, non-specific stimulation such as stimulation with concanavalin A, phytohemagglutinin-P, phorbol esters plus ionomycin, phorbol myristate acetate plus calcium ionophores, or antibodies having the ability to cross link TCRs (e.g., anti-CD3 antibodies plus anti-CD28 antibodies, or anti-TCR β antibodies) or antigen-specific stimulation such as stimulation with one or more particular antigens as described elsewhere (Downward et al., Nature, 346:719-23 (1990); and Dasgupta et al., Proc. Natl. Acad. Sci. USA, 84:1094-8 (1987)). In some cases, cytokine expression levels such as TNF-α, IFN-γ, IL-2, IL-4, IL-5, IL-10, IL-13, or IL-17 expression levels can be determined and compared to non-stimulated populations. Once single T cells are sorted, the methods provided herein can be used to determine which T cells were making particular cytokines in response to the stimulation (e.g., in response to a peptide antigen used to stimulate the T cells). In these cases, antigen specific T cells can be determined without laborious methods of expanding reactive T cells or the destructive methods of paraformaldehyde fixation and intracellular cytokine staining, which can reduce the ability to clone TCRs effectively. In such cases, particular TCRs generated from active and antigen specific T cells, as opposed to inactive bystander T cells, can be quickly identified.

In some cases, cytokine expression levels such as TNF-α, IFN-γ, IL-2, IL-4, IL-5, IL-10, IL-13, or IL-17 expression levels can be determined for the single T cells used to clone functional TCRs, thereby allowing a particular TCR to be identified based on the particular phenotype (e.g., elevated IFN-γ expression) of the T cell that provided the variable chains (or portions thereof) of that particular TCR. In such cases, particular TCRs generated from active, as opposed to inactive, T cells can be quickly identified. In some cases, particular TCRs generated from inactive, as opposed to active, T cells can be quickly identified.

In some cases, the absence of cytokine production by a T cell does not necessarily reflect an absence of TCR specificity. TCR initiated signals to a cell can be subverted and/or repressed by numerous inhibitory co-receptors (Sheppard et al., FEBS Lett., 574(1-3):37-41 (2004); and Yokosuka et al., J. Exp. Med., 209(6): 1201-1217 (2012)). In some cases, TCRs can be obtained using T cells refractory to stimulation, and the specificity of the cloned TCR can be tested or screened in cells where canonical TCR signaling is not repressed.

In some cases, a MHC-peptide complex (or an HLA-peptide complex) can be used to identify cloned TCRs that recognize such a complex. In these cases, it is possible that clonal exclusion during an immune response and/or a lack of antigen priming may result in TCRs with this specificity not being present in the activated and/or expanded TCR pool. In such cases, the methods and materials provided herein, which in some cases only requires a single T cell to be present, can be used to clone a naïve or inactivated TCR that recognizes such a complex. In some cases, pools of naïve T cells can be stained with MHC-peptide tetramers (or HLA-peptide tetramers), and any MHC-peptide (or HLA-peptide) responsive TCRs among the naïve T cells can be used to clone those TCRs using the methods and materials provided herein.

In some aspects, the methods provided herein include methods for obtaining a plurality of nucleic acid vectors containing nucleic acid encoding functional T cell receptors. The method comprises, or consists essentially of, (a) obtaining a device comprising a plurality of separate locations, wherein each of the separate locations contains cDNA generated from RNA obtained from a single T cell that was sorted into the separate locations, (b) performing a nested amplification procedure using the cDNA of each of the plurality of separate locations as template to obtain a first amplification product and a second amplification product for the cDNA of each of the plurality of separate locations, wherein the first amplification product comprises nucleic acid encoding α Vα or Vγ segment, and wherein the second amplification product comprises nucleic acid encoding α Vβ or Vδ segment, and (c) assembling the first amplification product and the second amplification product for the cDNA of each of the plurality of separate locations into a nucleic acid vector to obtain an assembled nucleic acid vector for the cDNA of each of the plurality of separate locations, wherein the assembled nucleic acid vectors for the cDNA of each of the plurality of separate locations comprises nucleic acid encoding a functional T cell receptor. The plurality can be greater than 50. The plurality can be greater than 500. The plurality can be greater than 5000. The plurality of nucleic acid vectors can be a plurality of nucleic acid expression vectors. The device can comprise a multi-well plate. The multi-well plate can be a 96-well plate, a 384-well plate, or a 1536-well plate. The cDNA generated from RNA obtained from a single T cell single can comprise cDNA generated from RNA obtained from a single human T cell. The first amplification product can comprise nucleic acid encoding an L sequence of α Vα or Vγ segment. The first amplification product can comprise nucleic acid encoding a Jα or Jγ segment. The first amplification product can comprise nucleic acid encoding a 5′ portion of a Cα or Cγ region. The first amplification product can comprise nucleic acid encoding an L sequence of α Vα or Vγ segment, a Jα or Jγ segment, and a 5′ portion of a Cα or Cγ region. The second amplification product can comprise nucleic acid encoding an L sequence of α Vβ or Vδ segment. The second amplification product can comprise nucleic acid encoding a DB or Dδ segment. The second amplification product can comprise nucleic acid encoding a Jβ or Jδ segment. The second amplification product can comprise nucleic acid encoding a 5′ portion of a Cβ or Cδ region. The second amplification product can comprise nucleic acid encoding an L sequence of α Vβ or Vδ segment, a Dβ or Dδ segment, a Jβ or Jδ segment, and a 5′ portion of a Cβ or Cδ region. The first amplification product can comprise an adapter sequence added to an amplified template sequence of the cDNA via a second round amplification of the nested amplification procedure. The second amplification product can comprise an adapter sequence added to an amplified template sequence of the cDNA via a second round amplification of the nested amplification procedure. The first amplification product can comprise a first adapter sequence added to an amplified template sequence of the cDNA via a second round amplification of the nested amplification procedure, and the second amplification product can comprise a second adapter sequence added to an amplified template sequence of the cDNA via a second round amplification of the nested amplification procedure, wherein the first and second adapter sequence are different. The functional T cell receptor of each of the assembled nucleic acid vectors can comprise a Vα/Vβ combination or Vγ/Vδ combination as present in the single T cell originating the RNA. The functional T cell receptor of each of the assembled nucleic acid vectors can comprise (a) a full-length a variable region and a full-length β variable region or (b) a full-length γ variable region and a full-length δ variable region. The functional T cell receptor of each of the assembled nucleic acid vectors can comprise (a) a full-length a variable region and a full-length β variable region as present in the single T cell originating the RNA or (b) a full-length γ variable region and a full-length δ variable region as present in the single T cell originating the RNA. The functional T cell receptor of each of the assembled nucleic acid vectors can comprise (a) a full-length a constant region and a full-length β constant region or (b) a full-length γ constant region and a full-length β constant region. Each of the assembled nucleic acid vectors can comprise a nucleic acid sequence encoding a self-cleaving peptide or an internal ribosome entry site (IRES). The method can comprise sorting single T cells into the separate locations. The method can comprise performing a reverse transcription reaction to obtain the cDNA. The assembling step can comprise seamless cloning. Each of the assembled nucleic acid vectors can be obtained without performing nucleic acid sequencing. Each of the assembled nucleic acid vectors can be obtained without performing a restriction endonuclease cleavage reaction.

### C. Assessing Minor Histocompatibility Antigen Specific T Cell Receptor Expression, Activity and Function

Exemplary assays can be used to assess the activity, expression and/or function of the TCRs and antigen-binding fragments described herein. The assays described herein, which are not to be construed as limiting, may be used to assess the functional capacity of candidate miHA-specific TCRs.

Functional characterization of TCRs is performed either by binding assays utilizing fluorescent labeled MHC molecules carrying specific target peptides (tetramer/pentamer/dextramer), or activation assays by co-culturing TCR expressing cells with antigen presenting cells (APCs) presenting the corresponding MHC/peptide complexes.

A cytokine release assay can evaluate the ability of a candidate TCR to produce the cytokines IL-2 and/or IFN-γ following exposure to cells presenting the target antigen. T cells are incubated with T2 cells (HA-1(neg)/HLA-A *: 02:01^{(pos)}) loaded with the target HA-1 “H” peptide. As a control, T2 cells are loaded with the non-target HA-1 “R” peptide or an irrelevant peptide control. IL-2 and/or IFN-γ responses of the T cells are followed by intracellular cytokine staining and analysis by FACS.

A T cell activation/degranulation marker assay can evaluate the ability of candidate TCRs to express the surface marker CD107a following exposure to cells presenting the target antigen. CD107a is a marker of T cell degranulation, which is part of the cell killing response. T cells are incubated with T2 cells loaded with the target HA-1 “H” peptide. As a control, T2 cells are loaded with the non-target HA-1 “R” peptide or an irrelevant peptide control. Degranulation responses are followed on the T cells by CD107a surface staining and analysis by FACS.

A killing assay can evaluate the ability of candidate TCRs to lyse cells presenting the target antigen. T cells are incubated with a mixture of fluorescent-tag labeled T2 cells differentially loaded with target and control peptides to allow on-target and off-target cytotoxicity to be examined within a single test sample. Fluorescent cell counting beads are included as a normalization/count control. Fluorescently tagged T2 cells are loaded with the target HA-1 “H” peptide. As a control, T2 cells labeled with a different fluorescent-tag are loaded with the non-target HA-1 “R” peptide or an irrelevant peptide control. Gated cell counts of HA-1 “H” peptide loaded T2 cells a control peptide loaded T2 cells remaining after incubation with T cells are followed by FACS.

The CD34 marker may be used as a surrogate potency measurement. See Philip et al. (2014) Blood 124(8): 1277-1287. Detection of CD34, a marker of transduction efficiency, may correlate with the functional potency measurements described herein.

In some aspects, expanded and unexpanded screens are performed using the exact same donor for a direct comparison of methods. With certain methods, expansion may be performed. A method that yields candidate TCRs without expansion processes can be advantageous in some contexts in view of the time sensitivity of screening for TCRs with desired specificity. Reduced sample processing may also offer advantages in different contexts.

### IV. Engineered Cells

Also provided herein are cells such as cells that have been engineered to contain a TCR described herein. Also provided herein are populations of such cells and compositions containing such cells and/or enriched for such cells, such as in which cells expressing the TCR make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition. In some embodiments, the cells are primary T cells or cells of a certain type such as T cells or CD8+ or CD4+ cells. Among the compositions are pharmaceutical compositions and formulations for administration, such as for adoptive cell therapy. Also provided herein are therapeutic methods for administering the cells and compositions to subjects, e.g., patients.

Thus also provided herein are genetically engineered cells expressing a TCR provided herein. The cells generally are eukaryotic cells, such as mammalian cells, and typically are human cells. In some embodiments, the cells are derived from the blood, bone marrow, lymph, or lymphoid organs, and are cells of the immune system, such as cells of the innate or adaptive immune system, e.g., myeloid or lymphoid cells, including lymphocytes, typically T cells and/or NK cells. Other exemplary cells include stem cells, such as multipotent and pluripotent stem cells, including induced pluripotent stem cells (iPSCs). The cells typically are primary cells, such as those isolated directly from a subject and/or isolated from a subject and frozen. In some embodiments, the cells include one or more subsets of T cells or other cell types, such as whole T cell populations, CD4+ cells, CD8+ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. With reference to the subject to be treated, the cells may be allogeneic and/or autologous. Among the methods provided herein include off-the-shelf methods. In some aspects, such as for off-the-shelf technologies, the cells are pluripotent and/or multipotent, such as stem cells (e.g., iPSCs). In some embodiments, the methods provided herein include isolating cells from the subject, preparing, processing, culturing, and/or engineering them, as described herein, and re-introducing them into the same patient, before or after cryopreservation.

Among the sub-types and subpopulations of T cells and/or of CD4+ and/or of CD8+ T cells included herein are naïve T (T_{N}) cells, effector T cells (T_{EFF}), memory T cells and sub-types thereof, such as stem cell memory T (T_{SCM}), central memory T (T_{CM}), effector memory T (TEM), or terminally differentiated effector memory T cells, tumor-infiltrating lymphocytes (TIL), immature T cells, mature T cells, helper T cells, cytotoxic T cells, mucosa-associated invariant T (MAIT) cells, naturally occurring and adaptive regulatory T (Treg) cells, helper T cells, such as TH1 cells, TH2 cells, TH3 cells, TH17 cells, TH9 cells, TH22 cells, follicular helper T cells, alpha/beta T cells, and delta/gamma T cells.

In some embodiments, the cells are NK cells. In some embodiments, the cells are monocytes or granulocytes, e.g., myeloid cells, macrophages, neutrophils, dendritic cells, mast cells, eosinophils, and/or basophils.

In some embodiments, the cells include one or more nucleic acids introduced via genetic engineering, and thereby express recombinant or genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature, including one comprising chimeric combinations of nucleic acids encoding various domains from multiple different cell types.

In some embodiments, the expression of the endogenous TCR chains of the engineered cell is reduced or eliminated, for example, to reduce the risk or chance of mispairing between chains of the engineered TCR and the endogenous TCR. Such mispairing could create a new TCR that could potentially result in a higher risk of undesired or unintended antigen recognition and/or side effects and/or could reduce expression levels of the desired exogenous TCR. Exemplary methods for reducing or preventing endogenous TCR expression are described elsewhere, see e.g. U.S. Pat. No. 9,273,283; U.S. publication no. US2014/0301990.

### A. Preparation of Cells for Genetic Engineering

In some embodiments, preparation of the engineered cells includes one or more culture and/or preparation steps. The cells for introduction of the TCR may be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, PBMCs, leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., T cell lines. The cells in some embodiments are obtained from a xenogeneic source, for example, from mouse, rat, non-human primate, or pig.

### B. Vectors and Methods for Genetic Engineering

Also provided herein are methods, nucleic acids, compositions, and kits for expressing a TCR or antigen-binding fragment thereof provided herein, and for producing the genetically engineered cells expressing such TCR or antigen-binding fragment thereof. The genetic engineering generally involves introduction of a nucleic acid encoding the TCR (or antigen-binding fragment thereof) into the cell, such as by retroviral transduction, transfection, or transformation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

Various methods for the introduction of genetically engineered components are well known and may be used with the provided methods and compositions. Exemplary methods include those for transfer of nucleic acids encoding a TCR or antigen-binding fragment thereof provided herein, including via viral, e.g., retroviral or lentiviral, transduction, transposons, and electroporation.

In some embodiments, recombinant nucleic acids are transferred into cells using recombinant infectious virus particles. In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 November 29(11): 550-557).

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the nucleic acid to be expressed replaces the retroviral gag, pol and/or env sequences. A number of illustrative retroviral systems have been described elsewhere (see, e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109).

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLOS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing nucleic acid provided herein in immune cells include calcium phosphate transfection (e.g., as described in Current Protocols in Molecular Biology, John Wiley δ Sons, New York. N.Y.), protoplast fusion, cationic liposome-mediated transfection, tungsten particle-facilitated microparticle bombardment (Johnston, Nature, 346: 776-777 (1990)), and strontium phosphate DNA co-precipitation (Brash et al., Mol. Cell Biol., 7: 2031-2034 (1987)).

Other approaches and vectors for transfer of nucleic acid encoding a TCR, antigen-binding fragment thereof, or recombinant product provided herein include those described elsewhere. See, e.g., International Patent Application Publication No. WO2014/055668 and U.S. Pat. No. 7,446,190.

In some cases, one or more additional nucleic acids can be introduced into a cell concurrently with or sequentially with nucleic acid encoding a TCR or antigen-binding fragment thereof provided herein. In some cases, such an additional nucleic acid for introduction can be those that improve the efficacy of therapy, such as by promoting viability and/or function of transferred cells; those that provide a genetic marker for selection and/or evaluation of the cells, such as to assess in vivo survival or localization; and/or those that improve safety, for example, by making the cell susceptible to negative selection in vivo as described elsewhere (Lupton S. D. et al., Mol. and Cell Biol., 11:6 (1991); and Riddell et al., Human Gene Therapy 3:319-338 (1992)). See, also, (a) the publications of PCT/US91/08442 and PCT/US94/05601 by Lupton et al. describing the use of bifunctional selectable fusion genes derived from fusing a dominant positive selectable marker with a negative selectable marker, and (b) Riddell et al., U.S. Pat. No. 6,040,177, at columns 14-17.

Thus, provided in some embodiments are engineered cells, such as those containing a TCR or antigen-binding fragment thereof, nucleic acid, or vector as described herein. In some aspects, the cell is produced by transducing the cell in vitro or ex vivo with a vector described herein. In some aspects, the cell is a T cell, such as a CD8+ or CD4+ T cell. In some embodiments, the TCR is heterologous to the cell.

### V. Therapeutic and Prophylactic Methods and Uses

Also provided herein are methods of administering and uses, such as therapeutic and prophylactic uses, of the TCRs and antigen-binding fragments thereof provided herein and/or engineered cells expressing the TCRs or antigen-binding fragments thereof. Such methods and uses include therapeutic methods and uses, for example, involving administration of the molecules, cells, or compositions containing the same, to a subject having a hematological disease, condition, or disorder to which alloSCT is a treatment option. In some embodiments, the molecule, cell, and/or composition is administered in an effective amount to effect treatment of the disease or disorder. Uses include uses of the TCRs and cells in such methods and treatments, and in the preparation of a medicament in order to carry out such therapeutic methods. In some embodiments, the methods are carried out by administering the TCRs or cells, or compositions comprising the same, to the subject having, having had, or suspected of having the disease or condition. In some embodiments, the methods thereby treat the disease or condition or disorder in the subject.

As used herein, “treatment” (and grammatical variations thereof such as “treat” or “treating”) refers to complete or partial amelioration or reduction of a disease or condition or disorder, or a symptom, adverse effect or outcome, or phenotype associated therewith. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. The terms do not imply complete curing of a disease or complete elimination of any symptom or effect(s) on all symptoms or outcomes.

As used herein, “delaying development of a disease” means to defer, hinder, slow, retard, stabilize, suppress and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

“Preventing,” as used herein, includes providing prophylaxis with respect to the occurrence or recurrence of a disease in a subject that may be predisposed to the disease but has not yet been diagnosed with the disease. In some embodiments, the provided molecules and compositions are used to delay development of a disease or to slow the progression of a disease.

As used herein, to “suppress” a function or activity is to reduce the function or activity when compared to otherwise same conditions except for a condition or parameter of interest, or alternatively, as compared to another condition. For example, a TCR or composition or cell which suppresses tumor growth reduces the rate of growth of the tumor compared to the rate of growth of the tumor in the absence of the TCR or composition or cell.

An “effective amount” of an agent, e.g., a pharmaceutical formulation, TCR, cells, or composition, in the context of administration, refers to an amount effective, at dosages/amounts and for periods of time necessary, to achieve a desired result, such as a therapeutic or prophylactic result.

A “therapeutically effective amount” of an agent, e.g., a pharmaceutical formulation, TCR, or cells, refers to an amount effective, at dosages and for periods of time necessary, to achieve a desired therapeutic result, such as for treatment of a disease, condition, or disorder, and/or pharmacokinetic or pharmacodynamic effect of the treatment. The therapeutically effective amount may vary according to factors such as the disease state, age, sex, and weight of the subject, and the populations of cells administered. In some embodiments, the provided methods involve administering the TCRs, cells, and/or compositions at effective amounts, e.g., therapeutically effective amounts.

A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

As used herein, a “subject” is a mammal, such as a human or other animal, and typically is human.

Among the diseases to be treated are cancers. In some embodiments, the disease or condition to be treated is a liquid tumor. In some embodiments, the disease or condication to be treated is a hematopoietic tumor. In some embodiments, the disease or condition to be treated is a lymphoma. In some embodiments, the disease or condition to be treated is acute myeloid leukemia (AML), a myelodysplastic syndrome (MDS), or acute lymphoblastic leukemia (ALL). In some embodiments, the disease or condition to be treated is chronic myeloid leukemia (CML).

### A. Exemplary Hematological Malignancies, Allogeneic Stem Cell Transplantation, and Patient Outcomes

### 1. Acute Myeloid Leukemia (AML)

Although AML is the most common indication for alloSCT, only half of the patients with early to intermediate disease and one third of patients with advanced disease survived at 3 years after transplant. See D'Souza et al. (2020) Biol Blood Marrow Transplant J Am Soc Blood Marrow Transplant 26(8): e177-e182. The most common cause of death in both early and late disease is relapse of primary disease. The recipient with overt active AML (i.e. >5% morphologically evident disease in the bone marrow) or measurable residual disease (MRD) at the time of alloSCT have a worse post-transplant prognosis than patients without MRD at the time of alloSCT. Methods for determining the presence or absence of MRD have evolved significantly and include evaluation for morphologic remission, multiparameter flow cytometry (MFC) and next-generation sequencing (NGS). MFC and NGS allow for determining presence of MRD down to 1×10⁴-1:10⁶ cells versus 1:20 in morphology-based determinations. See Schuurhuis et al. (2018) Blood 131(12): 1275-1291 and Getta et al. (2017) Biol Blood Marrow Transpl. 23(7): 1064-1071.

Longer term outcomes for patients with MRD are poor, with relapse occurring in 65% of subjects, resulting in RFS of 13% and overall survival (OS) of 19-23% at three years. See Araki et al. (2016) J Clin Oncol. 34(4):329-336 and Duval et al. (2010) J Clin Oncol. 28(23): 3730-3738. The three-year relapse rate in one retrospective study for MRD-positive patients was 67% (similar to the 65% relapse rate found in those with active AML), compared to 22% relapse in patients with MRD-negative remission. See Araki et al. (2016) J Clin Oncol. 34(4):329-336. Other published studies have yielded similar results. See Mohty et al. (2017) Haematologica. 102(1):184-191, Decroocq et al. (2018) Am J Hematol. 93(3):416-423, and Walter et al. (2013) Blood 122(10): 1813-1821. Despite very poor outcomes, one retrospective study demonstrated the benefit of alloSCT for MRD-positive patients in comparison to a no transplant option, chemotherapy. See Jurjen et al. (2017) JCO Precis Oncol. (1):1-13.

Therapies targeting specific mutations, such as IDH or FLT3 inhibitors, have been used in salvage regimens and increasingly up front. See Lai et al. (2019) J Hematol Oncol 12(1): 100. But despite these agents, which are active in only a minority of leukemias, relapsed/refractory disease will remain a major clinical problem. Likewise, post-transplant hypomethylating agents are currently used, but have an uncertain effect on long term survival. See Platzbecker et al. (2012) Leukemia 26(3):381-389, Craddock et al. (2019) J Clin Oncol Off J Am Soc Clin Oncol. 37(7):580-588, and Rautenberg et al. (2020) Bone Marrow Transplant 1-9.

AlloSCT remains the standard of care for AML with active disease or with MRD, despite a disappointing 60% of recipients relapsing during the first year, and less than one-third of patients becoming long-term survivors. See D'Souza et al. (2020) Biol Blood Marrow Transplant J Am Soc Blood Marrow Transplant 26(8):e177-e182. Most patients with overt disease are typically not offered alloSCT due to this likelihood of relapse during the first year. There is an urgent unmet medical need to extend the length of time before relapse (e.g., to extend the time to relapse beyond 1, 2, 3, 4, or 5 years) in MRD-positive patients who undergo alloSCT. There also is an urgent unmet medical need to prevent relapse in MRD-positive patients who undergo alloSCT.

### 2. Myelodysplastic Syndrome

The risk of having an MDS relapse after alloSCT is greater in patients transplanted with higher risk disease as measured by the Revised International Prognostic Scoring System (IPSS-R). Approximately 50-60% of very-poor risk MDS patients relapsed in 2 years after alloSCT. Monosomy cytogenetic abnormalities are also associated with a higher risk of relapse independent of IPSS score. See Koenecke et al. (2015) Haematologica. 100(3):400-408 and Deeg et al. (2012) Blood 120(7):1398-1408. More recently, specific somatic mutation profiles were shown to predict for relapse of MDS post-alloSCT. Pre-transplant TP53 mutations were associated with a very poor outcome with 3-year overall survival of less than 20% and a median survival time of 0.7 years. See Lindsley et al. (2017) N Engl J Med. 376(6):536-547 and Ciurea et al. (2018) Blood 131(26):2989-2992. Other mutations related to the RAS-pathway, JAK2, RUNX1, and ASXL1 were also associated with poor outcome after alloSCT. See Lindsley et al. (2017) N Engl J Med. 376(6):536-547 and Della Porta et al. (2016) J Clin Oncol. 34(30):3627-3637.

### 3. Acute Lymphoblastic Leukemia (ALL)

AlloSCT for ALL with active disease or primary induction failure only achieved 16% long-term survival at three years, as 41% of patients died before six months from relapsed ALL. See Duval et al. (2010) J Clin Oncol. 28(23):3730-3738.

### 4. Additional Diseased and Conditions

Additional diseases or condition can be treated using the described TCRs, T cells, and methods. The diseases or conditions include, but are not limited to, liquid tumors, hematopoietic tumors, lymphomas, and CML.

In some embodiments, the described described TCRs and T cells may be used in bone marrow transplantations, such as for autoimmune disorders, solid tumor treatments, and immune system replacements. The described T cells may be combined with other adoptive cell therapies that are targeted to solid tumors, or autoimmune diseases or conditions. The described T cells may be used as a preliminary or concurrent treatment or additive to reduce, inhibit, or eliminate the recipient's natural immune response or immune cells.

### B. Allogeneic Stem Cell Transplantation and Risk of Relapse

Over 9000 alloSCTs were performed in the United States in 2019, mostly as a potentially curative treatment for patients with various hematologic malignancies. See D'Souza et al. (2020) Biol Blood Marrow Transplant J Am Soc Blood Marrow Transplant 26(8):e177-e182. Post-transplant relapse remains the major cause of transplant failure occurring in 20-40% of standard risk and in 40-80% of high-risk patients, accounting for more than half of deaths after alloSCT. See Horowitz et al. (2018) Bone Marrow Transpl. 53(11): 1379-1389. There is an urgent need to prolong recurrence-free survival (RFS) times through new strategies to enhance GVL without causing severe GVHD. There also is an urgent need to prevent and treat post-transplant relapse through new strategies to enhance GVL without causing severe GVHD.

The number of relapses in patients currently transplanted likely underestimates the unmet needs. An analysis of patients with AML is illustrative of this point. In 2018, more than 3,000 alloSCTs were performed for AML in the United States. However, during the same period, there were approximately 21,450 new cases of AML, with an estimated 11,000 yearly deaths. The decision to refer a patient for an alloSCT depends on the benefit of relapse control relative to the risks of treatment-related mortality (TRM). If a new therapy results in a lower rate of relapse without an increase in significant toxicities and TRM, then more subjects would likely be referred for that therapy.

Relapse is the most common cause of death after alloSCT in every type of hematologic malignancy. Since the outcome of post-transplant relapse is extremely poor, RFS or cumulative relapse can be used as reliable surrogate endpoints for survival in alloSCT. The most powerful predictor for relapse is measurable residual disease (MRD) at the time of alloSCT. Even if the disease burden is low (i.e., less than 5% of the bone marrow), the outcomes are as poor as in the patients with overt active disease. See Araki et al. (2016) J Clin Oncol. 34(4):329-336.

### C. Additional Therapy Considerations

In some aspects, although unlikely, it is possible for miHA TCRs to display a lack of specificity or exhibit on target/off tumor effects. The latter may emerge when the HA-1 target is sufficiently expressed in nonhematopoietic tissues. Strategies and methods, which should not be construed as limiting, are provided herein to address such occurrences.

If GVHD occurs following administration of a miHA TCR, standard of care immunosuppressive therapies would be initiated. As a built in safety mechanism, the engineered cells described herein may comprise an extracellular membrane-bound marker containing a CD20 epitope. The CD20 epitope is recognized by certain antibodies, including, for example, the monoclonal antibody RITUXANR (rituximab). Recognition of the CD20 epitope may allow for selective deletion of the engineered cells. This strategy may be employed in combination with standard of care interventions to reduce GVHD.

While cytokine release syndrome (CRS) has occurred after CAR-T cell infusions, the risk of CRS is less likely for TCR cell therapy. Even so, CRS remains a possibility, especially if transduced cells are rapidly and synchronously activated. If CRS occurs, as defined by the American Society for Transplantation and Cellular Therapy (ASTCT) consensus grading guidelines, standard of care therapies would be initiated. Such treatments include, for example, administration of antibodies that block IL-6 function and/or corticosteroids. See Lee et al. (2019) Biol Blood Marrow Transpl. 25(4):625-638.

### VI. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polypeptide” and “protein” are used interchangeably to refer to a polymer of amino acid residues, and are not limited to a minimum length. Polypeptides, including the provided T cell receptors, antigen binding fragments thereof and other peptides, e.g., linkers, may include amino acid residues including natural and/or non-natural amino acid residues. The terms also include post-expression modifications of the polypeptide, for example, glycosylation, sialylation, acetylation, phosphorylation, and the like. In some aspects, the polypeptides may contain modifications with respect to a native or natural sequence, as long as the protein maintains the desired activity. These modifications may be deliberate, as through site-directed mutagenesis, or may be accidental, such as through mutations of hosts which produce the proteins or errors due to PCR amplification.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“An isolated nucleic acid molecule encoding a TCR” refers to a single nucleic acid molecule (e.g., single vector) that encodes a TCR such as a functional α/β TCR or a functional γ/δ TCR.

“An isolated nucleic acid molecule encoding an antigen binding fragment of a TCR” refers to a single nucleic acid molecule (e.g., single vector) that encodes an antigen binding fragment of a TCR.

“Isolated nucleic acid molecules encoding a TCR” refers to two or more separate nucleic acid molecules (e.g., two or more vectors) that together encode a TCR such as a functional α/β TCR or a functional γ/δ TCR. Each of such two or more nucleic acid molecules can be present at different locations within a host cell.

“Isolated nucleic acid molecules encoding an antigen binding fragment of a TCR” refers to two or more nucleic acid molecules (e.g., two or more vectors) that together encode an antigen binding fragment of a TCR. Each of such two or more nucleic acid molecules can be present at different locations within a host cell.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, “percent (%) amino acid sequence identity” and “percent identity” when used with respect to an amino acid sequence (reference polypeptide sequence) is defined as the percentage of amino acid residues in a candidate sequence (e.g., the subject T cell receptor or fragment) that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared.

An amino acid substitution may include replacement of one amino acid in a polypeptide with another amino acid. Amino acid substitutions may be introduced into a TCR or antigen binding fragment thereof, of interest and the products screened for a desired activity, e.g., retained/improved antigen binding, decreased immunogenicity, or improved cytolytic activity.

Amino acids generally can be grouped according to the following common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro; and
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations.

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be included in the smaller ranges, and are also encompassed within the claimed subject matter, subject to any specifically excluded limit in the stated range. Where the stated range includes one or both of the limits, ranges excluding either or both of those included limits are also included in the claimed subject matter. This applies regardless of the breadth of the range.

The term “about” as used herein refers to the usual error range for the respective value readily known to the skilled person in this technical field. Reference to “about” a value or parameter herein includes (and describes) embodiments that are directed to that value or parameter per se. For example, description referring to “about X” includes description of “X”

As used herein, a composition refers to any mixture of two or more products, substances, or compounds, including cells. It may be a solution, a suspension, liquid, powder, a paste, aqueous, non-aqueous or any combination thereof.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

### VII. Exemplary Embodiments

Among the provided embodiments are:
1. A T cell receptor (TCR) or antigen-binding fragment thereof, comprising:
an alpha chain comprising a variable alpha (Vα) region and a beta chain comprising a variable beta (Vβ) region; or
a gamma chain comprising a variable gamma (Vγ) region and a delta chain comprising a variable delta (Vδ) region; wherein:
(a) the Vα or Vγ region comprises a complementarity determining region 3 (CDR-3) comprising SEQ ID NO:3, and the Vβ or Vδ region comprises a CDR-3 comprising SEQ ID NO:11;
(b) the Vα or Vγ region comprises a CDR-3 comprising SEQ ID NO:21, and the Vβ or Vδ region comprises a CDR-3 comprising SEQ ID NO:27;
(c) the Vα or Vγ region comprises a CDR-3 comprising SEQ ID NO:37, and the Vβ or Vδ region comprises a CDR-3 comprising SEQ ID NO:43;
(d) the Vα or Vγ region comprises a CDR-3 comprising SEQ ID NO:51, and the Vβ or Vδ region comprises a CDR-3 comprising SEQ ID NO:57;
(e) the Vα or Vγ region comprises a CDR-3 comprising SEQ ID NO:65, and the Vβ or Vδ region comprises a CDR-3 comprising SEQ ID NO:57;
(f) the Vα or Vγ region comprises a CDR-3 comprising SEQ ID NO:78, and the Vβ or Vδ region comprises a CDR-3 comprising SEQ ID NO:84;
(g) the Vα or Vγ region comprises a CDR-3 comprising SEQ ID NO:92, and the Vβ or Vδ region comprises a CDR-3 comprising SEQ ID NO:98;
(h) the Vα or Vγ region comprises a CDR-3 comprising SEQ ID NO:106, and the Vβ or Vδ region comprises a CDR-3 comprising SEQ ID NO:112;
(i) the Vα or Vγ region comprises a CDR-3 comprising SEQ ID NO:120, and the Vβ or Vδ region comprises a CDR-3 comprising SEQ ID NO:126;
(j) the Vα or Vγ region comprises a CDR-3 comprising SEQ ID NO:136, and the Vβ or Vδ region comprises a CDR-3 comprising SEQ ID NO:142;
(k) the Vα or Vγ region comprises a CDR-3 comprising SEQ ID NO:152, and the Vβ or Vδ region comprises a CDR-3 comprising SEQ ID NO:158;
(l) the Vα or Vγ region comprises a CDR-3 comprising SEQ ID NO:166, and the Vβ or Vδ region comprises a CDR-3 comprising SEQ ID NO:172;
(m) the Vα or Vγ region comprises a CDR-3 comprising SEQ ID NO:180, and the Vβ or Vδ region comprises a CDR-3 comprising SEQ ID NO:186;
(n) the Vα or Vγ region comprises a CDR-3 comprising SEQ ID NO:194, and the Vβ or Vδ region comprises a CDR-3 comprising SEQ ID NO:200;
(o) the Vα or Vγ region comprises a CDR-3 comprising SEQ ID NO:208, and the Vβ or Vδ region comprises a CDR-3 comprising SEQ ID NO:214;
(p) the Vα or Vγ region comprises a CDR-3 comprising SEQ ID NO:224, and the Vβ or Vδ region comprises a CDR-3 comprising SEQ ID NO:230;
(q) the Vα or Vγ region comprises a CDR-3 comprising SEQ ID NO:238, and the Vβ or Vδ region comprises a CDR-3 comprising SEQ ID NO:244;
(r) the Vα or Vγ region comprises a CDR-3 comprising SEQ ID NO:252, and the Vβ or Vδ region comprises a CDR-3 comprising SEQ ID NO:258;
(s) the Vα or Vγ region comprises a CDR-3 comprising SEQ ID NO:268, and the Vβ or Vδ region comprises a CDR-3 comprising SEQ ID NO:158;
(t) the Vα or Vγ region comprises a CDR-3 comprising SEQ ID NO:278, and the Vβ or Vδ region comprises a CDR-3 comprising SEQ ID NO:284;
(u) the Vα or Vγ region comprises a CDR-3 comprising SEQ ID NO:359, and the Vβ or Vδ region comprises a CDR-3 comprising SEQ ID NO:363;
(v) the Vα or Vγ region comprises a CDR-3 comprising SEQ ID NO:369, and the Vβ or Vδ region comprises a CDR-3 comprising SEQ ID NO:373;
(w) the Vα or Vγ region comprises a CDR-3 comprising SEQ ID NO:379, and the Vβ or Vδ region comprises a CDR-3 comprising SEQ ID NO:383;
(x) the Vα or Vγ region comprises a CDR-3 comprising SEQ ID NO:389, and the Vβ or Vδ region comprises a CDR-3 comprising SEQ ID NO:393;
(y) the Vα or Vγ region comprises a CDR-3 comprising SEQ ID NO:399, and the Vβ or Vδ region comprises a CDR-3 comprising SEQ ID NO:403;
(z) the Vα or Vγ region comprises a CDR-3 comprising SEQ ID NO:409, and the Vβ or Vδ region comprises a CDR-3 comprising SEQ ID NO:413;
(aa) the Vα or Vγ region comprises a CDR-3 comprising SEQ ID NO:419, and the Vβ or Vδ region comprises a CDR-3 comprising SEQ ID NO:423;
(ab) the Vα or Vγ region comprises a CDR-3 comprising SEQ ID NO:429, and the Vβ or Vδ region comprises a CDR-3 comprising SEQ ID NO:433;
(ac) the Vα or Vγ region comprises a CDR-3 comprising SEQ ID NO:439, and the Vβ or Vδ region comprises a CDR-3 comprising SEQ ID NO:443;
(ad) the Vα or Vγ region comprises a CDR-3 comprising SEQ ID NO:449, and the Vβ or Vδ region comprises a CDR-3 comprising SEQ ID NO:453; or
(ac) the Vα or Vγ region comprises a CDR-3 comprising SEQ ID NO:480, and the Vβ or Vδ region comprises a CDR-3 comprising SEQ ID NO:484.
2. The TCR or antigen-binding fragment thereof of embodiment 1, wherein:
(a) the Vα or Vγ region comprises a complementarity determining region 1 (CDR-1) comprising SEQ ID NO:1, and a complementarity determining region 2 (CDR-2) comprising SEQ ID NO:2, and the Vβ or Vδ region comprises a CDR-1 comprising SEQ ID NO:9, and a CDR-2 comprising SEQ ID NO:10;
(b) the Vα or Vγ region comprises a CDR-1 comprising SEQ ID NO:19, and a CDR-2 comprising SEQ ID NO:20, and the Vβ or Vδ region comprises a CDR-1 comprising SEQ ID NO:9, and a CDR-2 comprising SEQ ID NO:10;
(c) the Vα or Vγ region comprises a CDR-1 comprising SEQ ID NO:35, and a CDR-2 comprising SEQ ID NO:36, and the Vβ or Vδ region comprises a CDR-1 comprising SEQ ID NO:9, and a CDR-2 comprising SEQ ID NO:10;
(d) the Vα or Vγ region comprises a CDR-1 comprising SEQ ID NO:1, and a CDR-2 comprising SEQ ID NO:2, and the Vβ or Vδ region comprises a CDR-1 comprising SEQ ID NO:9, and a CDR-2 comprising SEQ ID NO:10;
(e) the Vα or Vγ region comprises a CDR-1 comprising SEQ ID NO:1, and a CDR-2 comprising SEQ ID NO:2, and the Vβ or Vδ region comprises a CDR-1 comprising SEQ ID NO:9, and a CDR-2 comprising SEQ ID NO:10;
(f) the Vα or Vγ region comprises a CDR-1 comprising SEQ ID NO:76, and a CDR-2 comprising SEQ ID NO:77, and the Vβ or Vδ region comprises a CDR-1 comprising SEQ ID NO:9, and a CDR-2 comprising SEQ ID NO:10;
(g) the Vα or Vγ region comprises a CDR-1 comprising SEQ ID NO:19, and a CDR-2 comprising SEQ ID NO:20, and the Vβ or Vδ region comprises a CDR-1 comprising SEQ ID NO:9, and a CDR-2 comprising SEQ ID NO:10;
(h) the Vα or Vγ region comprises a CDR-1 comprising SEQ ID NO:76, and a CDR-2 comprising SEQ ID NO:77, and the Vβ or Vδ region comprises a CDR-1 comprising SEQ ID NO:9, and a CDR-2 comprising SEQ ID NO:10;
(i) the Vα or Vγ region comprises a CDR-1 comprising SEQ ID NO:35, and a CDR-2 comprising SEQ ID NO:36, and the Vβ or Vo region comprises a CDR-1 comprising SEQ ID NO:9, and a CDR-2 comprising SEQ ID NO:10;
(j) the Vα or Vγ region comprises a CDR-1 comprising SEQ ID NO:134, and a CDR-2 comprising SEQ ID NO:135, and the Vβ or Vδ region comprises a CDR-1 comprising SEQ ID NO:9, and a CDR-2 comprising SEQ ID NO:10;
(k) the Vα or Vγ region comprises a CDR-1 comprising SEQ ID NO:150, and a CDR-2 comprising SEQ ID NO:151, and the Vβ or Vo region comprises a CDR-1 comprising SEQ ID NO:9, and a CDR-2 comprising SEQ ID NO:10;
(l) the Vα or Vγ region comprises a CDR-1 comprising SEQ ID NO:35, and a CDR-2 comprising SEQ ID NO:36, and the Vβ or Vδ region comprises a CDR-1 comprising SEQ ID NO:9, and a CDR-2 comprising SEQ ID NO:10;
(m) the Vα or Vγ region comprises a CDR-1 comprising SEQ ID NO:35, and a CDR-2 comprising SEQ ID NO:36, and the Vβ or Vδ region comprises a CDR-1 comprising SEQ ID NO:9, and a CDR-2 comprising SEQ ID NO:10;
(n) the Vα or Vγ region comprises a CDR-1 comprising SEQ ID NO:76, and a CDR-2 comprising SEQ ID NO:77, and the Vβ or Vδ region comprises a CDR-1 comprising SEQ ID NO:9, and a CDR-2 comprising SEQ ID NO:10;
(o) the Vα or Vγ region comprises a CDR-1 comprising SEQ ID NO:76, and a CDR-2 comprising SEQ ID NO:77, and the Vβ or Vo region comprises a CDR-1 comprising SEQ ID NO:9, and a CDR-2 comprising SEQ ID NO:10;
(p) the Vα or Vγ region comprises a CDR-1 comprising SEQ ID NO:222, and a CDR-2 comprising SEQ ID NO:223, and the Vβ or Vδ region comprises a CDR-1 comprising SEQ ID NO:9, and a CDR-2 comprising SEQ ID NO: 10;
(q) the Vα or Vγ region comprises a CDR-1 comprising SEQ ID NO:76, and a CDR-2 comprising SEQ ID NO:77, and the Vβ or Vo region comprises a CDR-1 comprising SEQ ID NO:9, and a CDR-2 comprising SEQ ID NO:10;
(r) the Vα or Vγ region comprises a CDR-1 comprising SEQ ID NO:19, and a CDR-2 comprising SEQ ID NO:20, and the Vβ or Vδ region comprises a CDR-1 comprising SEQ ID NO:9, and a CDR-2 comprising SEQ ID NO:10;
(s) the Vα or Vγ region comprises a CDR-1 comprising SEQ ID NO:266, and a CDR-2 comprising SEQ ID NO:267, and the Vβ or Vδ region comprises a CDR-1 comprising SEQ ID NO:9, and a CDR-2 comprising SEQ ID NO:10; or
(t) the Vα or Vγ region comprises a CDR-1 comprising SEQ ID NO:19, and a CDR-2 comprising SEQ ID NO:20, and the Vβ or Vδ region comprises a CDR-1 comprising SEQ ID NO:9, and a CDR-2 comprising SEQ ID NO:10.
3. A T cell receptor (TCR) or antigen-binding fragment thereof, comprising:
an alpha chain comprising a variable alpha (Vα) region and a beta chain comprising a variable beta (Vβ) region; or
a gamma chain comprising a variable gamma (Vγ) region and a delta chain comprising a variable delta (Vδ) region; wherein:
(a) the Vα or Vγ region comprises a complementarity determining region 1 (CDR-1) comprising SEQ ID NO:1, a complementarity determining region 2 (CDR-2) comprising SEQ ID NO:2, and a complementarity determining region 3 (CDR-3) comprising SEQ ID NO:3, and the Vβ or Vδ region comprises a CDR-1 comprising SEQ ID NO:9, a CDR-2 comprising SEQ ID NO:10, and a CDR-3 comprising SEQ ID NO:11;
(b) the Vα or Vγ region comprises a CDR-1 comprising SEQ ID NO:19, a CDR-2 comprising SEQ ID NO:20, and a CDR-3 comprising SEQ ID NO:21, and the Vβ or Vδ region comprises a CDR-1 comprising SEQ ID NO:9, a CDR-2 comprising SEQ ID NO:10, and a CDR-3 comprising SEQ ID NO:27;
(c) the Vα or Vγ region comprises a CDR-1 comprising SEQ ID NO:35, a CDR-2 comprising SEQ ID NO:36, and a CDR-3 comprising SEQ ID NO:37, and the Vβ or Vδ region comprises a CDR-1 comprising SEQ ID NO:9, a CDR-2 comprising SEQ ID NO:10, and a CDR-3 comprising SEQ ID NO:43;
(d) the Vα or Vγ region comprises a CDR-1 comprising SEQ ID NO:1, a CDR-2 comprising SEQ ID NO:2, and a CDR-3 comprising SEQ ID NO:51, and the Vβ or Vδ region comprises a CDR-1 comprising SEQ ID NO:9, a CDR-2 comprising SEQ ID NO:10, and a CDR-3 comprising SEQ ID NO:57;
(e) the Vα or Vγ region comprises a CDR-1 comprising SEQ ID NO:1, a CDR-2 comprising SEQ ID NO:2, and a CDR-3 comprising SEQ ID NO:65, and the Vβ or Vδ region comprises a CDR-1 comprising SEQ ID NO:9, a CDR-2 comprising SEQ ID NO:10, and a CDR-3 comprising SEQ ID NO:57;
(f) the Vα or Vγ region comprises a CDR-1 comprising SEQ ID NO:76, a CDR-2 comprising SEQ ID NO:77, and a CDR-3 comprising SEQ ID NO:78, and the Vβ or Vδ region comprises a CDR-1 comprising SEQ ID NO:9, a CDR-2 comprising SEQ ID NO:10, and a CDR-3 comprising SEQ ID NO:84;
(g) the Vα or Vγ region comprises a CDR-1 comprising SEQ ID NO:19, a CDR-2 comprising SEQ ID NO:20, and a CDR-3 comprising SEQ ID NO:92, and the Vβ or Vδ region comprises a CDR-1 comprising SEQ ID NO:9, a CDR-2 comprising SEQ ID NO:10, and a CDR-3 comprising SEQ ID NO:98;
(h) the Vα or Vγ region comprises a CDR-1 comprising SEQ ID NO:76, a CDR-2 comprising SEQ ID NO:77, and a CDR-3 comprising SEQ ID NO:106, and the Vβ or Vδ region comprises a CDR-1 comprising SEQ ID NO:9, a CDR-2 comprising SEQ ID NO:10, and a CDR-3 comprising SEQ ID NO:112;
(i) the Vα or Vγ region comprises a CDR-1 comprising SEQ ID NO:35, a CDR-2 comprising SEQ ID NO:36, and a CDR-3 comprising SEQ ID NO:120, and the Vβ or Vδ region comprises a CDR-1 comprising SEQ ID NO:9, a CDR-2 comprising SEQ ID NO:10, and a CDR-3 comprising SEQ ID NO:126;
(j) the Vα or Vγ region comprises a CDR-1 comprising SEQ ID NO:134, a CDR-2 comprising SEQ ID NO:135, and a CDR-3 comprising SEQ ID NO:136, and the Vβ or Vδ region comprises a CDR-1 comprising SEQ ID NO:9, a CDR-2 comprising SEQ ID NO:10, and a CDR-3 comprising SEQ ID NO:142;
(k) the Vα or Vγ region comprises a CDR-1 comprising SEQ ID NO:150, a CDR-2 comprising SEQ ID NO:151, and a CDR-3 comprising SEQ ID NO:152, and the Vβ or Vδ region comprises a CDR-1 comprising SEQ ID NO:9, a CDR-2 comprising SEQ ID NO:10, and a CDR-3 comprising SEQ ID NO:158;
(l) the Vα or Vγ region comprises a CDR-1 comprising SEQ ID NO:35, a CDR-2 comprising SEQ ID NO:36, and a CDR-3 comprising SEQ ID NO:166, and the Vβ or Vδ region comprises a CDR-1 comprising SEQ ID NO:9, a CDR-2 comprising SEQ ID NO:10, and a CDR-3 comprising SEQ ID NO:172;
(m) the Vα or Vγ region comprises a CDR-1 comprising SEQ ID NO:35, a CDR-2 comprising SEQ ID NO:36, and a CDR-3 comprising SEQ ID NO:180, and the Vβ or Vδ region comprises a CDR-1 comprising SEQ ID NO:9, a CDR-2 comprising SEQ ID NO:10, and a CDR-3 comprising SEQ ID NO:186;
(n) the Vα or Vγ region comprises a CDR-1 comprising SEQ ID NO:76, a CDR-2 comprising SEQ ID NO:77, and a CDR-3 comprising SEQ ID NO:194, and the Vβ or Vδ region comprises a CDR-1 comprising SEQ ID NO:9, a CDR-2 comprising SEQ ID NO:10, and a CDR-3 comprising SEQ ID NO:200;
(o) the Vα or Vγ region comprises a CDR-1 comprising SEQ ID NO:76, a CDR-2 comprising SEQ ID NO:77, and a CDR-3 comprising SEQ ID NO:208, and the Vβ or Vδ region comprises a CDR-1 comprising SEQ ID NO:9, a CDR-2 comprising SEQ ID NO:10, and a CDR-3 comprising SEQ ID NO:214;
(p) the Vα or Vγ region comprises a CDR-1 comprising SEQ ID NO:222, a CDR-2 comprising SEQ ID NO:223, and a CDR-3 comprising SEQ ID NO:224, and the Vβ or Vδ region comprises a CDR-1 comprising SEQ ID NO:9, a CDR-2 comprising SEQ ID NO:10, and a CDR-3 comprising SEQ ID NO:230;
(q) the Vα or Vγ region comprises a CDR-1 comprising SEQ ID NO:76, a CDR-2 comprising SEQ ID NO:77, and a CDR-3 comprising SEQ ID NO:238, and the Vβ or Vδ region comprises a CDR-1 comprising SEQ ID NO:9, a CDR-2 comprising SEQ ID NO:10, and a CDR-3 comprising SEQ ID NO:244;
(r) the Vα or Vγ region comprises a CDR-1 comprising SEQ ID NO:19, a CDR-2 comprising SEQ ID NO:20, and a CDR-3 comprising SEQ ID NO:252, and the Vβ or Vδ region comprises a CDR-1 comprising SEQ ID NO:9, a CDR-2 comprising SEQ ID NO:10, and a CDR-3 comprising SEQ ID NO:258;
(s) the Vα or Vγ region comprises a CDR-1 comprising SEQ ID NO:266, a CDR-2 comprising SEQ ID NO:267, and a CDR-3 comprising SEQ ID NO:268, and the Vβ or Vδ region comprises a CDR-1 comprising SEQ ID NO:9, a CDR-2 comprising SEQ ID NO:10, and a CDR-3 comprising SEQ ID NO: 158; or
(t) the Vα or Vγ region comprises a CDR-1 comprising SEQ ID NO:19, a CDR-2 comprising SEQ ID NO:20, and a CDR-3 comprising SEQ ID NO:278, and the Vβ or Vδ region comprises a CDR-1 comprising SEQ ID NO:9, a CDR-2 comprising SEQ ID NO:10, and a CDR-3 comprising SEQ ID NO:284.
4. A T cell receptor (TCR) or antigen-binding fragment thereof, comprising:
   an alpha chain comprising a variable alpha (Vα) region and a beta chain comprising a variable beta (Vβ) region; or
   a gamma chain comprising a variable gamma (Vγ) region and a delta chain comprising a variable delta (Vδ) region; wherein:
(a) the Vα or Vγ region comprises a complementarity determining region 3 (CDR-3) comprising a CDR-3 contained within the Vα or Vγ region sequence of SEQ ID NO:4, and the Vβ or Vδ region comprises a CDR-3 comprising a CDR-3 contained within the Vβ or Vδ region sequence of SEQ ID NO:12;
(b) the Vα or Vγ region comprises a CDR-3 comprising a CDR-3 contained within the Vα or Vγ region sequence of SEQ ID NO:22, and the Vβ or Vδ region comprises a CDR-3 comprising a CDR-3 contained within the Vβ or Vδ region sequence of SEQ ID NO:28;
(c) the Vα or Vγ region comprises a CDR-3 comprising a CDR-3 contained within the Vα or Vγ region sequence of SEQ ID NO:38, and the Vβ or Vδ region comprises a CDR-3 comprising a CDR-3 contained within the Vβ or Vδ region sequence of SEQ ID NO:44;
(d) the Vα or Vγ region comprises a CDR-3 comprising a CDR-3 contained within the Vα or Vγ region sequence of SEQ ID NO:52, and the Vβ or Vδ region comprises a CDR-3 comprising a CDR-3 contained within the Vβ or Vδ region sequence of SEQ ID NO:58;
(e) the Vα or Vγ region comprises a CDR-3 comprising a CDR-3 contained within the Vα or Vγ region sequence of SEQ ID NO:66, and the Vβ or Vδ region comprises a CDR-3 comprising a CDR-3 contained within the Vβ or Vδ region sequence of SEQ ID NO:58;
(f) the Vα or Vγ region comprises a CDR-3 comprising a CDR-3 contained within the Vα or Vγ region sequence of SEQ ID NO:79, and the Vβ or Vδ region comprises a CDR-3 comprising a CDR-3 contained within the Vβ or Vδ region sequence of SEQ ID NO:85;
(g) the Vα or Vγ region comprises a CDR-3 comprising a CDR-3 contained within the Vα or Vγ region sequence of SEQ ID NO:93, and the Vβ or Vδ region comprises a CDR-3 comprising a CDR-3 contained within the Vβ or Vδ region sequence of SEQ ID NO:99;
(h) the Vα or Vγ region comprises a CDR-3 comprising a CDR-3 contained within the Vα or Vγ region sequence of SEQ ID NO:107, and the Vβ or Vδ region comprises a CDR-3 comprising a CDR-3 contained within the Vβ or Vδ region sequence of SEQ ID NO:113;
(i) the Vα or Vγ region comprises a CDR-3 comprising a CDR-3 contained within the Vα or Vγ region sequence of SEQ ID NO:121, and the Vβ or Vo region comprises a CDR-3 comprising a CDR-3 contained within the Vβ or Vδ region sequence of SEQ ID NO:127;
(j) the Vα or Vγ region comprises a CDR-3 comprising a CDR-3 contained within the Vα or Vγ region sequence of SEQ ID NO:137, and the Vβ or Vo region comprises a CDR-3 comprising a CDR-3 contained within the Vβ or Vδ region sequence of SEQ ID NO:143;
(k) the Vα or Vγ region comprises a CDR-3 comprising a CDR-3 contained within the Vα or Vγ region sequence of SEQ ID NO: 153, and the Vβ or Vδ region comprises a CDR-3 comprising a CDR-3 contained within the Vβ or Vδ region sequence of SEQ ID NO:159;
(l) the Vα or Vγ region comprises a CDR-3 comprising a CDR-3 contained within the Vα or Vγ region sequence of SEQ ID NO: 167, and the Vβ or Vδ region comprises a CDR-3 comprising a CDR-3 contained within the Vβ or Vδ region sequence of SEQ ID NO:173;
(m) the Vα or Vγ region comprises a CDR-3 comprising a CDR-3 contained within the Vα or Vγ region sequence of SEQ ID NO:181, and the Vβ or Vδ region comprises a CDR-3 comprising a CDR-3 contained within the Vβ or Vδ region sequence of SEQ ID NO:187;
(n) the Vα or Vγ region comprises a CDR-3 comprising a CDR-3 contained within the Vα or Vγ region sequence of SEQ ID NO:195, and the Vβ or Vδ region comprises a CDR-3 comprising a CDR-3 contained within the Vβ or Vδ region sequence of SEQ ID NO:201;
(o) the Vα or Vγ region comprises a CDR-3 comprising a CDR-3 contained within the Vα or Vγ region sequence of SEQ ID NO:209, and the Vβ or Vo region comprises a CDR-3 comprising a CDR-3 contained within the Vβ or Vδ region sequence of SEQ ID NO:215;
(p) the Vα or Vγ region comprises a CDR-3 comprising a CDR-3 contained within the Vα or Vγ region sequence of SEQ ID NO:225, and the Vβ or Vo region comprises a CDR-3 comprising a CDR-3 contained within the Vβ or Vδ region sequence of SEQ ID NO:231;
(q) the Vα or Vγ region comprises a CDR-3 comprising a CDR-3 contained within the Vα or Vγ region sequence of SEQ ID NO: 239, and the Vβ or Vo region comprises a CDR-3 comprising a CDR-3 contained within the Vβ or Vδ region sequence of SEQ ID NO:245;
(r) the Vα or Vγ region comprises a CDR-3 comprising a CDR-3 contained within the Vα or Vγ region sequence of SEQ ID NO:253, and the Vβ or Vo region comprises a CDR-3 comprising a CDR-3 contained within the Vβ or Vδ region sequence of SEQ ID NO:259;
(s) the Vα or Vγ region comprises a CDR-3 comprising a CDR-3 contained within the Vα or Vγ region sequence of SEQ ID NO:269, and the Vβ or Vo region comprises a CDR-3 comprising a CDR-3 contained within the Vβ or Vδ region sequence of SEQ ID NO: 159; or
(t) the Vα or Vγ region comprises a CDR-3 comprising a CDR-3 contained within the Vα or Vγ region sequence of SEQ ID NO:279, and the Vβ or Vo region comprises a CDR-3 comprising a CDR-3 contained within the Vβ or Vδ region sequence of SEQ ID NO:285.
5. The TCR or antigen-binding fragment thereof of embodiment 4, wherein:
(a) the Vα or Vγ region comprises a complementarity determining region 1 (CDR-1) and a complementarity determining region 2 (CDR-2) comprising a CDR-1 and a CDR-2, respectively, contained within the Vα or Vγ region sequence of SEQ ID NO:4, and the Vβ or Vδ region comprises a CDR-1 and a CDR-2 comprising a CDR-1 and a CDR-2, respectively, contained within the Vβ or Vδ region sequence of SEQ ID NO:12;
(b) the Vα or Vγ region comprises a CDR-1 and a CDR-2 comprising a CDR-1 and a CDR-2, respectively, contained within the Vα or Vγ region sequence of SEQ ID NO:22, and the Vβ or Vδ region comprises a CDR-1 and a CDR-2 comprising a CDR-1 and a CDR-2, respectively, contained within the Vβ or Vδ region sequence of SEQ ID NO:28;
(c) the Vα or Vγ region comprises a CDR-1 and a CDR-2 comprising a CDR-1 and a CDR-2, respectively, contained within the Vα or Vγ region sequence of SEQ ID NO:38, and the Vβ or Vo region comprises a CDR-1 and a CDR-2 comprising a CDR-1 and a CDR-2, respectively, contained within the Vβ or Vδ region sequence of SEQ ID NO:44;
(d) the Vα or Vγ region comprises a CDR-1 and a CDR-2 comprising a CDR-1 and a CDR-2, respectively, contained within the Vα or Vγ region sequence of SEQ ID NO:52, and the Vβ or Vδ region comprises a CDR-1 and a CDR-2 comprising a CDR-1 and a CDR-2, respectively, contained within the Vβ or Vδ region sequence of SEQ ID NO:58;
(e) the Vα or Vγ region comprises a CDR-1 and a CDR-2 comprising a CDR-1 and a CDR-2, respectively, contained within the Vα or Vγ region sequence of SEQ ID NO:66, and the Vβ or Vδ region comprises a CDR-1 and a CDR-2 comprising a CDR-1 and a CDR-2, respectively, contained within the Vβ or Vδ region sequence of SEQ ID NO:58;
(f) the Vα or Vγ region comprises a CDR-1 and a CDR-2 comprising a CDR-1 and a CDR-2, respectively, contained within the Vα or Vγ region sequence of SEQ ID NO:79, and the Vβ or Vo region comprises a CDR-1 and a CDR-2 comprising a CDR-1 and a CDR-2, respectively, contained within the Vβ or Vδ region sequence of SEQ ID NO:85;
(g) the Vα or Vγ region comprises a CDR-1 and a CDR-2 comprising a CDR-1 and a CDR-2, respectively, contained within the Vα or Vγ region sequence of SEQ ID NO:93, and the Vβ or Vδ region comprises a CDR-1 and a CDR-2 comprising a CDR-1 and a CDR-2, respectively, contained within the Vβ or Vδ region sequence of SEQ ID NO:99;
(h) the Vα or Vγ region comprises a CDR-1 and a CDR-2 comprising a CDR-1 and a CDR-2, respectively, contained within the Vα or Vγ region sequence of SEQ ID NO:107, and the Vβ or Vδ region comprises a CDR-1 and a CDR-2 comprising a CDR-1 and a CDR-2, respectively, contained within the Vβ or Vδ region sequence of SEQ ID NO:113;
(i) the Vα or Vγ region comprises a CDR-1 and a CDR-2 comprising a CDR-1 and a CDR-2, respectively, contained within the Vα or Vγ region sequence of SEQ ID NO:121, and the Vβ or Vo region comprises a CDR-1 and a CDR-2 comprising a CDR-1 and a CDR-2, respectively, contained within the Vβ or Vδ region sequence of SEQ ID NO:127;
(j) the Vα or Vγ region comprises a CDR-1 and a CDR-2 comprising a CDR-1 and a CDR-2, respectively, contained within the Vα or Vγ region sequence of SEQ ID NO:137, and the Vβ or Vδ region comprises a CDR-1 and a CDR-2 comprising a CDR-1 and a CDR-2, respectively, contained within the Vβ or Vδ region sequence of SEQ ID NO:143;
(k) the Vα or Vγ region comprises a CDR-1 and a CDR-2 comprising a CDR-1 and a CDR-2, respectively, contained within the Vα or Vγ region sequence of SEQ ID NO:153, and the Vβ or Vδ region comprises a CDR-1 and a CDR-2 comprising a CDR-1 and a CDR-2, respectively, contained within the Vβ or Vδ region sequence of SEQ ID NO:159;
(l) the Vα or Vγ region comprises a CDR-1 and a CDR-2 comprising a CDR-1 and a CDR-2, respectively, contained within the Vα or Vγ region sequence of SEQ ID NO:167, and the Vβ or Vδ region comprises a CDR-1 and a CDR-2 comprising a CDR-1 and a CDR-2, respectively, contained within the Vβ or Vδ region sequence of SEQ ID NO:173;
(m) the Vα or Vγ region comprises a CDR-1 and a CDR-2 comprising a CDR-1 and a CDR-2, respectively, contained within the Vα or Vγ region sequence of SEQ ID NO:181, and the Vβ or Vδ region comprises a CDR-1 and a CDR-2 comprising a CDR-1 and a CDR-2, respectively, contained within the Vβ or Vδ region sequence of SEQ ID NO:187;
(n) the Vα or Vγ region comprises a CDR-1 and a CDR-2 comprising a CDR-1 and a CDR-2, respectively, contained within the Vα or Vγ region sequence of SEQ ID NO:195, and the Vβ or Vδ region comprises a CDR-1 and a CDR-2 comprising a CDR-1 and a CDR-2, respectively, contained within the Vβ or Vδ region sequence of SEQ ID NO:201;
(o) the Vα or Vγ region comprises a CDR-1 and a CDR-2 comprising a CDR-1 and a CDR-2, respectively, contained within the Vα or Vγ region sequence of SEQ ID NO:209, and the Vβ or Vδ region comprises a CDR-1 and a CDR-2 comprising a CDR-1 and a CDR-2, respectively, contained within the Vβ or Vδ region sequence of SEQ ID NO:215;
(p) the Vα or Vγ region comprises a CDR-1 and a CDR-2 comprising a CDR-1 and a CDR-2, respectively, contained within the Vα or Vγ region sequence of SEQ ID NO:225, and the Vβ or Vδ region comprises a CDR-1 and a CDR-2 comprising a CDR-1 and a CDR-2, respectively, contained within the Vβ or Vδ region sequence of SEQ ID NO:231;
(q) the Vα or Vγ region comprises a CDR-1 and a CDR-2 comprising a CDR-1 and a CDR-2, respectively, contained within the Vα or Vγ region sequence of SEQ ID NO:239, and the Vβ or Vδ region comprises a CDR-1 and a CDR-2 comprising a CDR-1 and a CDR-2, respectively, contained within the Vβ or Vδ region sequence of SEQ ID NO:245;
(r) the Vα or Vγ region comprises a CDR-1 and a CDR-2 comprising a CDR-1 and a CDR-2, respectively, contained within the Vα or Vγ region sequence of SEQ ID NO:253, and the Vβ or Vδ region comprises a CDR-1 and a CDR-2 comprising a CDR-1 and a CDR-2, respectively, contained within the Vβ or Vδ region sequence of SEQ ID NO:259;
(s) the Vα or Vγ region comprises a CDR-1 and a CDR-2 comprising a CDR-1 and a CDR-2, respectively, contained within the Vα or Vγ region sequence of SEQ ID NO:269, and the Vβ or Vδ region comprises a CDR-1 and a CDR-2 comprising a CDR-1 and a CDR-2, respectively, contained within the Vβ or Vδ region sequence of SEQ ID NO:159; or
(t) the Vα or Vγ region comprises a CDR-1 and a CDR-2 comprising a CDR-1 and a CDR-2, respectively, contained within the Vα or Vγ region sequence of SEQ ID NO:279, and the Vβ or Vδ region comprises a CDR-1 and a CDR-2 comprising a CDR-1 and a CDR-2, respectively, contained within the Vβ or Vδ region sequence of SEQ ID NO:285.
6. A T cell receptor (TCR) or antigen-binding fragment thereof, comprising:
an alpha chain comprising a variable alpha (Vα) region and a beta chain comprising a variable beta (Vβ) region; or
a gamma chain comprising a variable gamma (Vγ) region and a delta chain comprising a variable delta (Vδ) region; wherein:
(a) the Vα or Vγ region comprises a complementarity determining region 1 (CDR-1), a complementarity determining region 2 (CDR-2), and a complementarity determining region 3 (CDR-3) comprising a CDR-1, a CDR-2, and a CDR-3, respectively, contained within the Vα or Vγ region sequence of SEQ ID NO:4, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:12;
(b) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:22, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:28;
(c) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:38, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:44;
(d) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:52, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:58;
(e) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:66, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:58;
(f) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:79, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:85;
(g) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:93, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:99;
(h) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:107, and the Vβ or Vo region comprises a CDR-1, a CDR-2, and a CDR-3 comprising a CDR-1, a CDR-2, and a CDR-3, respectively, contained within the Vβ or Vδ region sequence of SEQ ID NO:113;
(i) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:121, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:127;
(j) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:137, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:143;
(k) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:153, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:159;
(l) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:167, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:173;
(m) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:181, and the Vβ or Vo region comprises a CDR-1, a CDR-2, and a CDR-3 comprising a CDR-1, a CDR-2, and a CDR-3, respectively, contained within the Vβ or Vδ region sequence of SEQ ID NO:187;
(n) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO: 195, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:201;
(o) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:209, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:215;
(p) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:225, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:231;
(q) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:239, and the Vβ or Vo region comprises a CDR-1, a CDR-2, and a CDR-3 comprising a CDR-1, a CDR-2, and a CDR-3, respectively, contained within the Vβ or Vδ region sequence of SEQ ID NO:245;
(r) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:253, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:259;
(s) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:269, and the Vβ or Vo region comprises a CDR-1, a CDR-2, and a CDR-3 comprising a CDR-1, a CDR-2, and a CDR-3, respectively, contained within the Vβ or Vδ region sequence of SEQ ID NO:159; or
(t) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:279, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:285.
7. A T cell receptor (TCR) or antigen-binding fragment thereof, comprising:
an alpha chain comprising a variable alpha (Vα) region and a beta chain comprising a variable beta (Vβ) region; or a gamma chain comprising a variable gamma (Vγ) region and a delta chain comprising a variable delta (Vδ) region; wherein:
(a) the Vα or Vγ region comprises SEQ ID NO:4 or a sequence that has at least 90% sequence identity thereto, and the Vβ or Vδ region comprises SEQ ID NO:12 or a sequence that has at least 90% sequence identity thereto;
(b) the Vα or Vγ region comprises SEQ ID NO:22 or a sequence that has at least 90% sequence identity thereto, and the Vβ or Vδ region comprises SEQ ID NO:28 or a sequence that has at least 90% sequence identity thereto;
(c) the Vα or Vγ region comprises SEQ ID NO:38 or a sequence that has at least 90% sequence identity thereto, and the Vβ or Vδ region comprises SEQ ID NO:44 or a sequence that has at least 90% sequence identity thereto;
(d) the Vα or Vγ region comprises SEQ ID NO:52 or a sequence that has at least 90% sequence identity thereto, and the Vβ or Vδ region comprises SEQ ID NO:58 or a sequence that has at least 90% sequence identity thereto;
(e) the Vα or Vγ region comprises SEQ ID NO:66 or a sequence that has at least 90% sequence identity thereto, and the Vβ or Vδ region comprises SEQ ID NO:58 or a sequence that has at least 90% sequence identity thereto;
(f) the Vα or Vγ region comprises SEQ ID NO:79 or a sequence that has at least 90% sequence identity thereto, and the Vβ or Vo region comprises SEQ ID NO:85 or a sequence that has at least 90% sequence identity thereto;
(g) the Vα or Vγ region comprises SEQ ID NO:93 or a sequence that has at least 90% sequence identity thereto, and the Vβ or Vo region comprises SEQ ID NO:99 or a sequence that has at least 90% sequence identity thereto;
(h) the Vα or Vγ region comprises SEQ ID NO:107 or a sequence that has at least 90% sequence identity thereto, and the Vβ or Vo region comprises SEQ ID NO:113 or a sequence that has at least 90% sequence identity thereto;
(i) the Vα or Vγ region comprises SEQ ID NO:121 or a sequence that has at least 90% sequence identity thereto, and the Vβ or Vo region comprises SEQ ID NO:127 or a sequence that has at least 90% sequence identity thereto;
(j) the Vα or Vγ region comprises SEQ ID NO:137 or a sequence that has at least 90% sequence identity thereto, and the Vβ or Vo region comprises SEQ ID NO: 143 or a sequence that has at least 90% sequence identity thereto;
(k) the Vα or Vγ region comprises SEQ ID NO:153 or a sequence that has at least 90% sequence identity thereto, and the Vβ or Vo region comprises SEQ ID NO:159 or a sequence that has at least 90% sequence identity thereto;
(l) the Vα or Vγ region comprises SEQ ID NO:167 or a sequence that has at least 90% sequence identity thereto, and the Vβ or Vo region comprises SEQ ID NO: 173 or a sequence that has at least 90% sequence identity thereto;
(m) the Vα or Vγ region comprises SEQ ID NO:181 or a sequence that has at least 90% sequence identity thereto, and the Vβ or Vo region comprises SEQ ID NO:187 or a sequence that has at least 90% sequence identity thereto;
(n) the Vα or Vγ region comprises SEQ ID NO: 195 or a sequence that has at least 90% sequence identity thereto, and the Vβ or Vδ region comprises SEQ ID NO:201 or a sequence that has at least 90% sequence identity thereto;
(o) the Vα or Vγ region comprises SEQ ID NO:209 or a sequence that has at least 90% sequence identity thereto, and the Vβ or Vo region comprises SEQ ID NO:215 or a sequence that has at least 90% sequence identity thereto;
(p) the Vα or Vγ region comprises SEQ ID NO:225 or a sequence that has at least 90% sequence identity thereto, and the Vβ or Vo region comprises SEQ ID NO:231 or a sequence that has at least 90% sequence identity thereto;
(q) the Vα or Vγ region comprises SEQ ID NO:239 or a sequence that has at least 90% sequence identity thereto, and the Vβ or Vo region comprises SEQ ID NO:245 or a sequence that has at least 90% sequence identity thereto;
(r) the Vα or Vγ region comprises SEQ ID NO:253 or a sequence that has at least 90% sequence identity thereto, and the Vβ or Vo region comprises SEQ ID NO:259 or a sequence that has at least 90% sequence identity thereto;
(s) the Vα or Vγ region comprises SEQ ID NO:269 or a sequence that has at least 90% sequence identity thereto, and the Vβ or Vo region comprises SEQ ID NO:159 or a sequence that has at least 90% sequence identity thereto; or
(t) the Vα or Vγ region comprises SEQ ID NO:279 or a sequence that has at least 90% sequence identity thereto, and the Vβ or Vo region comprises SEQ ID NO:285 or a sequence that has at least 90% sequence identity thereto.
8. The TCR or antigen-binding fragment thereof of any of embodiments 1-7, wherein:
(a) the Vα or Vγ region comprises SEQ ID NO:4, and the Vβ or Vδ region comprises SEQ ID NO:12;
(b) the Vα or Vγ region comprises SEQ ID NO:22, and the Vβ or Vδ region comprises SEQ ID NO:28;
(c) the Vα or Vγ region comprises SEQ ID NO:38, and the Vβ or Vδ region comprises SEQ ID NO:44;
(d) the Vα or Vγ region comprises SEQ ID NO:52, and the Vβ or Vδ region comprises SEQ ID NO:58;
(e) the Vα or Vγ region comprises SEQ ID NO:66, and the Vβ or Vδ region comprises SEQ ID NO:58;
(f) the Vα or Vγ region comprises SEQ ID NO:79, and the Vβ or Vδ region comprises SEQ ID NO:85;
(g) the Vα or Vγ region comprises SEQ ID NO:93, and the Vβ or Vδ region comprises SEQ ID NO:99;
(h) the Vα or Vγ region comprises SEQ ID NO:107, and the Vβ or Vδ region comprises SEQ ID NO:113;
(i) the Vα or Vγ region comprises SEQ ID NO:121, and the Vβ or Vδ region comprises SEQ ID NO:127;
(j) the Vα or Vγ region comprises SEQ ID NO:137, and the Vβ or Vδ region comprises SEQ ID NO:143;
(k) the Vα or Vγ region comprises SEQ ID NO:153, and the Vβ or Vδ region comprises SEQ ID NO:159;
(l) the Vα or Vγ region comprises SEQ ID NO:167, and the Vβ or Vδ region comprises SEQ ID NO:173;
(m) the Vα or Vγ region comprises SEQ ID NO:181, and the Vβ or Vδ region comprises SEQ ID NO:187;
(n) the Vα or Vγ region comprises SEQ ID NO:195, and the Vβ or Vδ region comprises SEQ ID NO:201;
(o) the Vα or Vγ region comprises SEQ ID NO:209, and the Vβ or Vδ region comprises SEQ ID NO:215;
(p) the Vα or Vγ region comprises SEQ ID NO:225, and the Vβ or Vδ region comprises SEQ ID NO:231;
(q) the Vα or Vγ region comprises SEQ ID NO:239, and the Vβ or Vδ region comprises SEQ ID NO:245;
(r) the Vα or Vγ region comprises SEQ ID NO:253, and the Vβ or Vδ region comprises SEQ ID NO:259;
(s) the Vα or Vγ region comprises SEQ ID NO:269, and the Vβ or Vδ region comprises SEQ ID NO:159; or
(t) the Vα or Vγ region comprises SEQ ID NO:279, and the Vβ or Vδ region comprises SEQ ID NO:285.
9. The TCR or antigen-binding fragment thereof of any of embodiments 1-8, wherein:
the alpha chain further comprises an alpha constant (Cα) region and the beta chain further comprises a beta constant (Cβ) region; or
the gamma chain further comprises an gamma constant (Cγ) region and the delta chain further comprises a delta constant (Cδ) region.
10. The TCR or antigen-binding fragment thereof of embodiment 9, wherein:
the Cα comprises SEQ ID NO: 294 or 296 and the Cβ comprises SEQ ID NO: 297 or 299.
11. The TCR or antigen-binding fragment thereof of any of embodiments 1-10, wherein:
(a) the alpha or gamma chain comprises SEQ ID NO:6, and the beta or delta chain comprises SEQ ID NO:14;
(b) the alpha or gamma chain comprises SEQ ID NO:24, and the beta or delta chain comprises SEQ ID NO:30;
(c) the alpha or gamma chain comprises SEQ ID NO:40, and the beta or delta chain comprises SEQ ID NO:46;
(d) the alpha or gamma chain comprises SEQ ID NO:54, and the beta or delta chain comprises SEQ ID NO:60;
(e) the alpha or gamma chain comprises SEQ ID NO:68, and the beta or delta chain comprises SEQ ID NO:71;
(f) the alpha or gamma chain comprises SEQ ID NO:81, and the beta or delta chain comprises SEQ ID NO:87;
(g) the alpha or gamma chain comprises SEQ ID NO:95, and the beta or delta chain comprises SEQ ID NO:101;
(h) the alpha or gamma chain comprises SEQ ID NO:109, and the beta or delta chain comprises SEQ ID NO:115;
(i) the alpha or gamma chain comprises SEQ ID NO:123, and the beta or delta chain comprises SEQ ID NO:129;
(j) the alpha or gamma chain comprises SEQ ID NO:139, and the beta or delta chain comprises SEQ ID NO:145;
(k) the alpha or gamma chain comprises SEQ ID NO:155, and the beta or delta chain comprises SEQ ID NO:161;
(l) the alpha or gamma chain comprises SEQ ID NO:169, and the beta or delta chain comprises SEQ ID NO:175;
(m) the alpha or gamma chain comprises SEQ ID NO:183, and the beta or delta chain comprises SEQ ID NO:189;
(n) the alpha or gamma chain comprises SEQ ID NO:197, and the beta or delta chain comprises SEQ ID NO:203;
(o) the alpha or gamma chain comprises SEQ ID NO:211, and the beta or delta chain comprises SEQ ID NO:217;
(p) the alpha or gamma chain comprises SEQ ID NO:227, and the beta or delta chain comprises SEQ ID NO:233;
(q) the alpha or gamma chain comprises SEQ ID NO:241, and the beta or delta chain comprises SEQ ID NO:247;
(r) the alpha or gamma chain comprises SEQ ID NO:255, and the beta or delta chain comprises SEQ ID NO:261;
(s) the alpha or gamma chain comprises SEQ ID NO:271, and the beta or delta chain comprises SEQ ID NO:161;
(t) the alpha or gamma chain comprises SEQ ID NO:281, and the beta or delta chain comprises SEQ ID NO:287;
(u) the alpha or gamma chain comprises SEQ ID NO:362, and the beta or delta chain comprises SEQ ID NO:366;
(v) the alpha or gamma chain comprises SEQ ID NO:372, and the beta or delta chain comprises SEQ ID NO:376;
(w) the alpha or gamma chain comprises SEQ ID NO:382, and the beta or delta chain comprises SEQ ID NO:386;
(x) the alpha or gamma chain comprises SEQ ID NO:392, and the beta or delta chain comprises SEQ ID NO:396;
(y) the alpha or gamma chain comprises SEQ ID NO:402, and the beta or delta chain comprises SEQ ID NO:406;
(z) the alpha or gamma chain comprises SEQ ID NO:412, and the beta or delta chain comprises SEQ ID NO:416;
(aa) the alpha or gamma chain comprises SEQ ID NO:422, and the beta or delta chain comprises SEQ ID NO:426;
(ab) the alpha or gamma chain comprises SEQ ID NO:432, and the beta or delta chain comprises SEQ ID NO:436;
(ac) the alpha or gamma chain comprises SEQ ID NO:442, and the beta or delta chain comprises SEQ ID NO:446;
(ad) the alpha or gamma chain comprises SEQ ID NO:452, and the beta or delta chain comprises SEQ ID NO:456; or (ae) the alpha or gamma chain comprises SEQ ID NO:483, and the beta or delta chain comprises SEQ ID NO:487.
12. The TCR or antigen-binding fragment thereof of any of embodiments 1-11, wherein the TCR or antigen-binding fragment thereof recognizes a peptide epitope of a minor histocompatibility antigen HA-1 in the context of an MHC molecule.
13. The TCR or antigen-binding fragment thereof of embodiment 12, wherein the MHC molecule is a human leukocyte antigens (HLA)-A molecule.
14. The TCR or antigen-binding fragment thereof of embodiment 13, wherein the HLA-A molecule is of serotype HLA-A*02:01.
15. The TCR or antigen-binding fragment thereof of embodiment 13, wherein the HLA-A molecule is of serotype HLA-A*02:06.
16. The TCR or antigen-binding fragment thereof of any of embodiments 12-15, wherein the peptide epitope of HA-1 is set forth in SEQ ID NO:354.
17. A polynucleotide encoding the TCR or antigen-binding fragment thereof of any of embodiments 1-16, or an alpha chain, a beta chain, a gamma chain, or a delta chain thereof.
18. The polynucleotide of embodiment 17, wherein the polynucleotide comprises a nucleotide sequence encoding the Vα region and a nucleotide sequence encoding the Vβ region; or a nucleotide sequence encoding the Vγ region and a nucleotide sequence encoding the Vδ region; wherein:
(a) the nucleotide sequence encoding the Vα or Vγ region comprises SEQ ID NO:7 or a sequence that has at least 90% sequence identity thereto, and the nucleotide sequence encoding the Vβ or Vδ region comprises SEQ ID NO:15 or a sequence that has at least 90% sequence identity thereto;
(b) the nucleotide sequence encoding the Vα or Vγ region comprises SEQ ID NO:25 or a sequence that has at least 90% sequence identity thereto, and the nucleotide sequence encoding the Vβ or Vδ region comprises SEQ ID NO:31 or a sequence that has at least 90% sequence identity thereto;
(c) the nucleotide sequence encoding the Vα or Vγ region comprises SEQ ID NO:41 or a sequence that has at least 90% sequence identity thereto, and the nucleotide sequence encoding the Vβ or Vδ region comprises SEQ ID NO:47 or a sequence that has at least 90% sequence identity thereto;
(d) the nucleotide sequence encoding the Vα or Vγ region comprises SEQ ID NO:55 or a sequence that has at least 90% sequence identity thereto, and the nucleotide sequence encoding the Vβ or Vδ region comprises SEQ ID NO:61 or a sequence that has at least 90% sequence identity thereto;
(e) the nucleotide sequence encoding the Vα or Vγ region comprises SEQ ID NO:69 or a sequence that has at least 90% sequence identity thereto, and the nucleotide sequence encoding the Vβ or Vδ region comprises SEQ ID NO:72 or a sequence that has at least 90% sequence identity thereto;
(f) the nucleotide sequence encoding the Vα or Vγ region comprises SEQ ID NO:82 or a sequence that has at least 90% sequence identity thereto, and the nucleotide sequence encoding the Vβ or Vδ region comprises SEQ ID NO:88 or a sequence that has at least 90% sequence identity thereto;
(g) the nucleotide sequence encoding the Vα or Vγ region comprises SEQ ID NO:96 or a sequence that has at least 90% sequence identity thereto, and the nucleotide sequence encoding the Vβ or Vδ region comprises SEQ ID NO:102 or a sequence that has at least 90% sequence identity thereto;
(h) the nucleotide sequence encoding the Vα or Vγ region comprises SEQ ID NO:110 or a sequence that has at least 90% sequence identity thereto, and the nucleotide sequence encoding the Vβ or Vδ region comprises SEQ ID NO:116 or a sequence that has at least 90% sequence identity thereto;
(i) the nucleotide sequence encoding the Vα or Vγ region comprises SEQ ID NO:124 or a sequence that has at least 90% sequence identity thereto, and the nucleotide sequence encoding the Vβ or Vδ region comprises SEQ ID NO:130 or a sequence that has at least 90% sequence identity thereto;
(j) the nucleotide sequence encoding the Vα or Vγ region comprises SEQ ID NO:140 or a sequence that has at least 90% sequence identity thereto, and the nucleotide sequence encoding the Vβ or Vδ region comprises SEQ ID NO:146 or a sequence that has at least 90% sequence identity thereto;
(k) the nucleotide sequence encoding the Vα or Vγ region comprises SEQ ID NO:156 or a sequence that has at least 90% sequence identity thereto, and the nucleotide sequence encoding the Vβ or Vδ region comprises SEQ ID NO:162 or a sequence that has at least 90% sequence identity thereto;
(l) the nucleotide sequence encoding the Vα or Vγ region comprises SEQ ID NO:170 or a sequence that has at least 90% sequence identity thereto, and the nucleotide sequence encoding the Vβ or Vδ region comprises SEQ ID NO:176 or a sequence that has at least 90% sequence identity thereto;
(m) the nucleotide sequence encoding the Vα or Vγ region comprises SEQ ID NO:184 or a sequence that has at least 90% sequence identity thereto, and the nucleotide sequence encoding the Vβ or Vδ region comprises SEQ ID NO:190 or a sequence that has at least 90% sequence identity thereto;
(n) the nucleotide sequence encoding the Vα or Vγ region comprises SEQ ID NO:198 or a sequence that has at least 90% sequence identity thereto, and the nucleotide sequence encoding the Vβ or Vδ region comprises SEQ ID NO:204 or a sequence that has at least 90% sequence identity thereto;
(o) the nucleotide sequence encoding the Vα or Vγ region comprises SEQ ID NO:212 or a sequence that has at least 90% sequence identity thereto, and the nucleotide sequence encoding the Vβ or Vδ region comprises SEQ ID NO:218 or a sequence that has at least 90% sequence identity thereto;
(p) the nucleotide sequence encoding the Vα or Vγ region comprises SEQ ID NO:228 or a sequence that has at least 90% sequence identity thereto, and the nucleotide sequence encoding the Vβ or Vδ region comprises SEQ ID NO:234 or a sequence that has at least 90% sequence identity thereto;
(q) the nucleotide sequence encoding the Vα or Vγ region comprises SEQ ID NO:242 or a sequence that has at least 90% sequence identity thereto, and the nucleotide sequence encoding the Vβ or Vδ region comprises SEQ ID NO:248 or a sequence that has at least 90% sequence identity thereto;
(r) the nucleotide sequence encoding the Vα or Vγ region comprises SEQ ID NO:256 or a sequence that has at least 90% sequence identity thereto, and the nucleotide sequence encoding the Vβ or Vδ region comprises SEQ ID NO:262 or a sequence that has at least 90% sequence identity thereto;
(s) the nucleotide sequence encoding the Vα or Vγ region comprises SEQ ID NO:272 or a sequence that has at least 90% sequence identity thereto, and the nucleotide sequence encoding the Vβ or Vδ region comprises SEQ ID NO:274 or a sequence that has at least 90% sequence identity thereto; or
(t) the nucleotide sequence encoding the Vα or Vγ region comprises SEQ ID NO:282 or a sequence that has at least 90% sequence identity thereto, and the nucleotide sequence encoding the Vβ or Vδ region comprises SEQ ID NO:288 or a sequence that has at least 90% sequence identity thereto.
19. The polynucleotide of embodiment 17 or 18, wherein:
(a) the nucleotide sequence encoding the alpha or gamma chain comprises SEQ ID NO:8, and the nucleotide sequence encoding the beta or delta chain comprises SEQ ID NO:16;
(b) the nucleotide sequence encoding the alpha or gamma chain comprises SEQ ID NO:26, and the nucleotide sequence encoding the beta or delta chain comprises SEQ ID NO:32;
(c) the nucleotide sequence encoding the alpha or gamma chain comprises SEQ ID NO:42, and the nucleotide sequence encoding the beta or delta chain comprises SEQ ID NO:48;
(d) the nucleotide sequence encoding the alpha or gamma chain comprises SEQ ID NO:56, and the nucleotide sequence encoding the beta or delta chain comprises SEQ ID NO:62;
(e) the nucleotide sequence encoding the alpha or gamma chain comprises SEQ ID NO:70, and the nucleotide sequence encoding the beta or delta chain comprises SEQ ID NO:73;
(f) the nucleotide sequence encoding the alpha or gamma chain comprises SEQ ID NO:83, and the nucleotide sequence encoding the beta or delta chain comprises SEQ ID NO:89;
(g) the nucleotide sequence encoding the alpha or gamma chain comprises SEQ ID NO:97, and the nucleotide sequence encoding the beta or delta chain comprises SEQ ID NO: 103;
(h) the nucleotide sequence encoding the alpha or gamma chain comprises SEQ ID NO:111, and the nucleotide sequence encoding the beta or delta chain comprises SEQ ID NO:117;
(i) the nucleotide sequence encoding the alpha or gamma chain comprises SEQ ID NO:125, and the nucleotide sequence encoding the beta or delta chain comprises SEQ ID NO: 131;
(j) the nucleotide sequence encoding the alpha or gamma chain comprises SEQ ID NO:141, and the nucleotide sequence encoding the beta or delta chain comprises SEQ ID NO: 147;
(k) the nucleotide sequence encoding the alpha or gamma chain comprises SEQ ID NO:157, and the nucleotide sequence encoding the beta or delta chain comprises SEQ ID NO:163;
(l) the nucleotide sequence encoding the alpha or gamma chain comprises SEQ ID NO:171, and the nucleotide sequence encoding the beta or delta chain comprises SEQ ID NO: 177;
(m) the nucleotide sequence encoding the alpha or gamma chain comprises SEQ ID NO:185, and the nucleotide sequence encoding the beta or delta chain comprises SEQ ID NO:191;
(n) the nucleotide sequence encoding the alpha or gamma chain comprises SEQ ID NO: 199, and the nucleotide sequence encoding the beta or delta chain comprises SEQ ID NO:205;
(o) the nucleotide sequence encoding the alpha or gamma chain comprises SEQ ID NO:213, and the nucleotide sequence encoding the beta or delta chain comprises SEQ ID NO:219;
(p) the nucleotide sequence encoding the alpha or gamma chain comprises SEQ ID NO:229, and the nucleotide sequence encoding the beta or delta chain comprises SEQ ID NO:235;
(q) the nucleotide sequence encoding the alpha or gamma chain comprises SEQ ID NO:243, and the nucleotide sequence encoding the beta or delta chain comprises SEQ ID NO:249;
(r) the nucleotide sequence encoding the alpha or gamma chain comprises SEQ ID NO:257, and the nucleotide sequence encoding the beta or delta chain comprises SEQ ID NO:263;
(s) the nucleotide sequence encoding the alpha or gamma chain comprises SEQ ID NO:273, and the nucleotide sequence encoding the beta or delta chain comprises SEQ ID NO:275; or
(t) the nucleotide sequence encoding the alpha or gamma chain comprises SEQ ID NO:283, and the nucleotide sequence encoding the beta or delta chain comprises SEQ ID NO:289.
20. The polynucleotide of embodiment 17 or 18, wherein:
(a) the nucleotide sequence encoding the Vα or Vγ region comprises SEQ ID NO:301, and the nucleotide sequence encoding the Vβ or Vδ region comprises SEQ ID NO:321;
(b) the nucleotide sequence encoding the Vα or Vγ region comprises SEQ ID NO:302, and the nucleotide sequence encoding the Vβ or Vδ region comprises SEQ ID NO:322;
(c) the nucleotide sequence encoding the Vα or Vγ region comprises SEQ ID NO:303, and the nucleotide sequence encoding the Vβ or Vδ region comprises SEQ ID NO:323;
(d) the nucleotide sequence encoding the Vα or Vγ region comprises SEQ ID NO:304, and the nucleotide sequence encoding the Vβ or Vδ region comprises SEQ ID NO:324;
(e) the nucleotide sequence encoding the Vα or Vγ region comprises SEQ ID NO:305, and the nucleotide sequence encoding the Vβ or Vδ region comprises SEQ ID NO:325;
(f) the nucleotide sequence encoding the Vα or Vγ region comprises SEQ ID NO:306, and the nucleotide sequence encoding the Vβ or Vδ region comprises SEQ ID NO:326;
(g) the nucleotide sequence encoding the Vα or Vγ region comprises SEQ ID NO:307, and the nucleotide sequence encoding the Vβ or Vδ region comprises SEQ ID NO:327;
(h) the nucleotide sequence encoding the Vα or Vγ region comprises SEQ ID NO:308, and the nucleotide sequence encoding the Vβ or Vδ region comprises SEQ ID NO:328;
(i) the nucleotide sequence encoding the Vα or Vγ region comprises SEQ ID NO:309, and the nucleotide sequence encoding the Vβ or Vδ region comprises SEQ ID NO:329;
(j) the nucleotide sequence encoding the Vα or Vγ region comprises SEQ ID NO:310, and the nucleotide sequence encoding the Vβ or Vδ region comprises SEQ ID NO:330;
(k) the nucleotide sequence encoding the Vα or Vγ region comprises SEQ ID NO:311, and the nucleotide sequence encoding the Vβ or Vδ region comprises SEQ ID NO:331;
(l) the nucleotide sequence encoding the Vα or Vγ region comprises SEQ ID NO:312, and the nucleotide sequence encoding the Vβ or Vδ region comprises SEQ ID NO:332;
(m) the nucleotide sequence encoding the Vα or Vγ region comprises SEQ ID NO:313, and the nucleotide sequence encoding the Vβ or Vδ region comprises SEQ ID NO:333;
(n) the nucleotide sequence encoding the Vα or Vγ region comprises SEQ ID NO:314, and the nucleotide sequence encoding the Vβ or Vδ region comprises SEQ ID NO:334;
(o) the nucleotide sequence encoding the Vα or Vγ region comprises SEQ ID NO:315, and the nucleotide sequence encoding the Vβ or Vδ region comprises SEQ ID NO:335;
(p) the nucleotide sequence encoding the Vα or Vγ region comprises SEQ ID NO:316, and the nucleotide sequence encoding the Vβ or Vδ region comprises SEQ ID NO:336;
(q) the nucleotide sequence encoding the Vα or Vγ region comprises SEQ ID NO:317, and the nucleotide sequence encoding the Vβ or Vδ region comprises SEQ ID NO:337;
(r) the nucleotide sequence encoding the Vα or Vγ region comprises SEQ ID NO:318, and the nucleotide sequence encoding the Vβ or Vδ region comprises SEQ ID NO:338;
(s) the nucleotide sequence encoding the Vα or Vγ region comprises SEQ ID NO:319, and the nucleotide sequence encoding the Vβ or Vδ region comprises SEQ ID NO:339; or
(t) the nucleotide sequence encoding the Vα or Vγ region comprises SEQ ID NO:320, and the nucleotide sequence encoding the Vβ or Vδ region comprises SEQ ID NO:340.
21. The polynucleotide of embodiment 17 or 18, wherein the polynucleotide comprises a nucleotide sequence encoding an alpha chain and a nucleotide sequence encoding a beta chain; or a nucleotide sequence encoding a gamma chain and a nucleotide sequence encoding a delta chain; wherein:
(a) the nucleotide sequence encoding the alpha or gamma chain comprises SEQ ID NO:8 or a sequence that has at least 90% sequence identity thereto, and the nucleotide sequence encoding the beta or delta chain comprises SEQ ID NO:16 or a sequence that has at least 90% sequence identity thereto;
(b) the nucleotide sequence encoding the alpha or gamma chain comprises SEQ ID NO:26 or a sequence that has at least 90% sequence identity thereto, and the nucleotide sequence encoding the beta or delta chain comprises SEQ ID NO:32 or a sequence that has at least 90% sequence identity thereto;
(c) the nucleotide sequence encoding the alpha or gamma chain comprises SEQ ID NO:42 or a sequence that has at least 90% sequence identity thereto, and the nucleotide sequence encoding the beta or delta chain comprises SEQ ID NO:48 or a sequence that has at least 90% sequence identity thereto;
(d) the nucleotide sequence encoding the alpha or gamma chain comprises SEQ ID NO:56 or a sequence that has at least 90% sequence identity thereto, and the nucleotide sequence encoding the beta or delta chain comprises SEQ ID NO:62 or a sequence that has at least 90% sequence identity thereto;
(e) the nucleotide sequence encoding the alpha or gamma chain comprises SEQ ID NO:70 or a sequence that has at least 90% sequence identity thereto, and the nucleotide sequence encoding the beta or delta chain comprises SEQ ID NO:73 or a sequence that has at least 90% sequence identity thereto;
(f) the nucleotide sequence encoding the alpha or gamma chain comprises SEQ ID NO:83 or a sequence that has at least 90% sequence identity thereto, and the nucleotide sequence encoding the beta or delta chain comprises SEQ ID NO:89 or a sequence that has at least 90% sequence identity thereto;
(g) the nucleotide sequence encoding the alpha or gamma chain comprises SEQ ID NO:97 or a sequence that has at least 90% sequence identity thereto, and the nucleotide sequence encoding the beta or delta chain comprises SEQ ID NO:103 or a sequence that has at least 90% sequence identity thereto;
(h) the nucleotide sequence encoding the alpha or gamma chain comprises SEQ ID NO:111 or a sequence that has at least 90% sequence identity thereto, and the nucleotide sequence encoding the beta or delta chain comprises SEQ ID NO:117 or a sequence that has at least 90% sequence identity thereto;
(i) the nucleotide sequence encoding the alpha or gamma chain comprises SEQ ID NO:125 or a sequence that has at least 90% sequence identity thereto, and the nucleotide sequence encoding the beta or delta chain comprises SEQ ID NO:131 or a sequence that has at least 90% sequence identity thereto;
(j) the nucleotide sequence encoding the alpha or gamma chain comprises SEQ ID NO:141 or a sequence that has at least 90% sequence identity thereto, and the nucleotide sequence encoding the beta or delta chain comprises SEQ ID NO:147 or a sequence that has at least 90% sequence identity thereto;
(k) the nucleotide sequence encoding the alpha or gamma chain comprises SEQ ID NO:157 or a sequence that has at least 90% sequence identity thereto, and the nucleotide sequence encoding the beta or delta chain comprises SEQ ID NO:163 or a sequence that has at least 90% sequence identity thereto;
(l) the nucleotide sequence encoding the alpha or gamma chain comprises SEQ ID NO:171 or a sequence that has at least 90% sequence identity thereto, and the nucleotide sequence encoding the beta or delta chain comprises SEQ ID NO:177 or a sequence that has at least 90% sequence identity thereto;
(m) the nucleotide sequence encoding the alpha or gamma chain comprises SEQ ID NO:185 or a sequence that has at least 90% sequence identity thereto, and the nucleotide sequence encoding the beta or delta chain comprises SEQ ID NO:191 or a sequence that has at least 90% sequence identity thereto;
(n) the nucleotide sequence encoding the alpha or gamma chain comprises SEQ ID NO:199 or a sequence that has at least 90% sequence identity thereto, and the nucleotide sequence encoding the beta or delta chain comprises SEQ ID NO:205 or a sequence that has at least 90% sequence identity thereto;
(o) the nucleotide sequence encoding the alpha or gamma chain comprises SEQ ID NO:213 or a sequence that has at least 90% sequence identity thereto, and the nucleotide sequence encoding the beta or delta chain comprises SEQ ID NO:219 or a sequence that has at least 90% sequence identity thereto;
(p) the nucleotide sequence encoding the alpha or gamma chain comprises SEQ ID NO:229 or a sequence that has at least 90% sequence identity thereto, and the nucleotide sequence encoding the beta or delta chain comprises SEQ ID NO:235 or a sequence that has at least 90% sequence identity thereto;
(q) the nucleotide sequence encoding the alpha or gamma chain comprises SEQ ID NO:243 or a sequence that has at least 90% sequence identity thereto, and the nucleotide sequence encoding the beta or delta chain comprises SEQ ID NO:249 or a sequence that has at least 90% sequence identity thereto;
(r) the nucleotide sequence encoding the alpha or gamma chain comprises SEQ ID NO:257 or a sequence that has at least 90% sequence identity thereto, and the nucleotide sequence encoding the beta or delta chain comprises SEQ ID NO:263 or a sequence that has at least 90% sequence identity thereto;
(s) the nucleotide sequence encoding the alpha or gamma chain comprises SEQ ID NO:273 or a sequence that has at least 90% sequence identity thereto, and the nucleotide sequence encoding the beta or delta chain comprises SEQ ID NO:275 or a sequence that has at least 90% sequence identity thereto; or
(t) the nucleotide sequence encoding the alpha or gamma chain comprises SEQ ID NO:283 or a sequence that has at least 90% sequence identity thereto, and the nucleotide sequence encoding the beta or delta chain comprises SEQ ID NO:289 or a sequence that has at least 90% sequence identity thereto.
22. The polynucleotide of embodiment 21, wherein:
(a) the nucleotide sequence encoding the alpha or gamma chain comprises SEQ ID NO:8, and the nucleotide sequence encoding the beta or delta chain comprises SEQ ID NO: 16;
(b) the nucleotide sequence encoding the alpha or gamma chain comprises SEQ ID NO:26, and the nucleotide sequence encoding the beta or delta chain comprises SEQ ID NO:32;
(c) the nucleotide sequence encoding the alpha or gamma chain comprises SEQ ID NO:42, and the nucleotide sequence encoding the beta or delta chain comprises SEQ ID NO:48;
(d) the nucleotide sequence encoding the alpha or gamma chain comprises SEQ ID NO:56, and the nucleotide sequence encoding the beta or delta chain comprises SEQ ID NO:62;
(e) the nucleotide sequence encoding the alpha or gamma chain comprises SEQ ID NO:70, and the nucleotide sequence encoding the beta or delta chain comprises SEQ ID NO:73;
(f) the nucleotide sequence encoding the alpha or gamma chain comprises SEQ ID NO:83, and the nucleotide sequence encoding the beta or delta chain comprises SEQ ID NO:89;
(g) the nucleotide sequence encoding the alpha or gamma chain comprises SEQ ID NO:97, and the nucleotide sequence encoding the beta or delta chain comprises SEQ ID NO: 103;
(h) the nucleotide sequence encoding the alpha or gamma chain comprises SEQ ID NO:111, and the nucleotide sequence encoding the beta or delta chain comprises SEQ ID NO:117;
(i) the nucleotide sequence encoding the alpha or gamma chain comprises SEQ ID NO:125, and the nucleotide sequence encoding the beta or delta chain comprises SEQ ID NO: 131;
(j) the nucleotide sequence encoding the alpha or gamma chain comprises SEQ ID NO:141, and the nucleotide sequence encoding the beta or delta chain comprises SEQ ID NO: 147;
(k) the nucleotide sequence encoding the alpha or gamma chain comprises SEQ ID NO:157, and the nucleotide sequence encoding the beta or delta chain comprises SEQ ID NO:163;
(l) the nucleotide sequence encoding the alpha or gamma chain comprises SEQ ID NO:171, and the nucleotide sequence encoding the beta or delta chain comprises SEQ ID NO:177;
(m) the nucleotide sequence encoding the alpha or gamma chain comprises SEQ ID NO:185, and the nucleotide sequence encoding the beta or delta chain comprises SEQ ID NO:191;
(n) the nucleotide sequence encoding the alpha or gamma chain comprises SEQ ID NO: 199, and the nucleotide sequence encoding the beta or delta chain comprises SEQ ID NO:205;
(o) the nucleotide sequence encoding the alpha or gamma chain comprises SEQ ID NO:213, and the nucleotide sequence encoding the beta or delta chain comprises SEQ ID NO:219;
(p) the nucleotide sequence encoding the alpha or gamma chain comprises SEQ ID NO: 229, and the nucleotide sequence encoding the beta or delta chain comprises SEQ ID NO:235;
(q) the nucleotide sequence encoding the alpha or gamma chain comprises SEQ ID NO:243, and the nucleotide sequence encoding the beta or delta chain comprises SEQ ID NO:249;
(r) the nucleotide sequence encoding the alpha or gamma chain comprises SEQ ID NO:257, and the nucleotide sequence encoding the beta or delta chain comprises SEQ ID NO:263;
(s) the nucleotide sequence encoding the alpha or gamma chain comprises SEQ ID NO:273, and the nucleotide sequence encoding the beta or delta chain comprises SEQ ID NO:275; or
(t) the nucleotide sequence encoding the alpha or gamma chain comprises SEQ ID NO:283, and the nucleotide sequence encoding the beta or delta chain comprises SEQ ID NO:289.
23. The polynucleotide of any of embodiments 17-22, wherein the nucleotide sequence encoding the alpha chain and the nucleotide sequence encoding the beta chain are separated by a peptide sequence that causes ribosome skipping.
24. The polynucleotide of embodiment 23, wherein the peptide that causes ribosome skipping is a P2A peptide.
25. The polynucleotide of embodiment 24, wherein the P2A peptide comprises SEQ ID NO:352.
26. The polynucleotide of embodiment 24 or 25, wherein the sequence encoding the P2A peptide is set forth in SEQ ID NO:351.
27. The polynucleotide of any one of embodiments 17-26, wherein:
(a) the nucleotide sequence encodes SEQ ID NO:18;
(b) the nucleotide sequence encodes SEQ ID NO:34;
(c) the nucleotide sequence encodes SEQ ID NO:50;
(d) the nucleotide sequence encodes SEQ ID NO:64;
(e) the nucleotide sequence encodes SEQ ID NO:75;
(f) the nucleotide sequence encodes SEQ ID NO:91;
(g) the nucleotide sequence encodes SEQ ID NO:105;
(h) the nucleotide sequence encodes SEQ ID NO:119;
(i) the nucleotide sequence encodes SEQ ID NO:133;
(j) the nucleotide sequence encodes SEQ ID NO:149;
(k) the nucleotide sequence encodes SEQ ID NO:165;
(l) the nucleotide sequence encodes SEQ ID NO:179;
(m) the nucleotide sequence encodes SEQ ID NO:193;
(n) the nucleotide sequence encodes SEQ ID NO:207;
(o) the nucleotide sequence encodes SEQ ID NO:221;
(p) the nucleotide sequence encodes SEQ ID NO:237;
(q) the nucleotide sequence encodes SEQ ID NO:251;
(r) the nucleotide sequence encodes SEQ ID NO:265;
(s) the nucleotide sequence encodes SEQ ID NO:277;
(t) the nucleotide sequence encodes SEQ ID NO:291;
(u) the nucleotide sequence encodes SEQ ID NO: 367;
(v) the nucleotide sequence encodes SEQ ID NO: 377;
(w) the nucleotide sequence encodes SEQ ID NO: 387;
(x) the nucleotide sequence encodes SEQ ID NO: 397;
(y) the nucleotide sequence encodes SEQ ID NO: 407;
(z) the nucleotide sequence encodes SEQ ID NO: 417;
(aa) the nucleotide sequence encodes SEQ ID NO: 427;
(ab) the nucleotide sequence encodes SEQ ID NO: 437;
(ac) the nucleotide sequence encodes SEQ ID NO: 447;
(ad) the nucleotide sequence encodes SEQ ID NO: 457; or
(ae) the nucleotide sequence encodes SEQ ID NO: 488.

28. The polynucleotide of any one of embodiments 17-27, wherein:
(a) the nucleotide sequence comprises SEQ ID NO:17;
(b) the nucleotide sequence comprises SEQ ID NO:33;
(c) the nucleotide sequence comprises SEQ ID NO:49;
(d) the nucleotide sequence comprises SEQ ID NO:63;
(e) the nucleotide sequence comprises SEQ ID NO:74;
(f) the nucleotide sequence comprises SEQ ID NO:90;
(g) the nucleotide sequence comprises SEQ ID NO:104;
(h) the nucleotide sequence comprises SEQ ID NO:118;
(i) the nucleotide sequence comprises SEQ ID NO:132;
(j) the nucleotide sequence comprises SEQ ID NO:148;
(k) the nucleotide sequence comprises SEQ ID NO:164;
(l) the nucleotide sequence comprises SEQ ID NO:178;
(m) the nucleotide sequence comprises SEQ ID NO:192;
(n) the nucleotide sequence comprises SEQ ID NO:206;
(o) the nucleotide sequence comprises SEQ ID NO:220;
(p) the nucleotide sequence comprises SEQ ID NO:236;
(q) the nucleotide sequence comprises SEQ ID NO:250;
(r) the nucleotide sequence comprises SEQ ID NO:264;
(s) the nucleotide sequence comprises SEQ ID NO:276;
(t) the nucleotide sequence comprises SEQ ID NO:290;
(u) the nucleotide sequence comprises SEQ ID NO: 368;
(v) the nucleotide sequence comprises SEQ ID NO: 378;
(w) the nucleotide sequence comprises SEQ ID NO: 388;
(x) the nucleotide sequence comprises SEQ ID NO: 398;
(y) the nucleotide sequence comprises SEQ ID NO: 408;
(z) the nucleotide sequence comprises SEQ ID NO: 418;
(aa) the nucleotide sequence comprises SEQ ID NO: 428;
(ab) the nucleotide sequence comprises SEQ ID NO: 438;
(ac) the nucleotide sequence comprises SEQ ID NO: 448;
(ad) the nucleotide sequence comprises SEQ ID NO: 458; or
(ae) the nucleotide sequence comprises SEQ ID NO: 489.
29. A vector comprising the nucleic acid of any of embodiments 17-28.
30. The vector of embodiment 29, wherein the vector is a viral vector.
31. The vector of embodiment 30, wherein the viral vector is a lentiviral vector.
32. An engineered cell, comprising the TCR or antigen-binding fragment thereof of any of embodiments 1-16.
33. An engineered cell, comprising the polynucleotide of any of embodiments 17-28 or the vector of any of embodiments 29-31.
34. The engineered cell of embodiment 32 or 33, wherein the TCR or antigen-binding fragment thereof is heterologous to the cell.
35. The engineered cell of any of embodiments 32-34, wherein the engineered cell is a cell line.
36. The engineered cell of any of embodiments 32-34, wherein the engineered cell is a primary cell obtained from a subject.
37. The engineered cell of any of embodiments 32-36, wherein the engineered cell is a T cell.
38. A method for producing an engineered cell, comprising introducing the polynucleotide of any of embodiments 17-28 or the vector of any of embodiments 29-31 into a cell in vitro or ex vivo.
39. A composition comprising the TCR or antigen-binding fragment thereof of any of embodiments 1-16, the polynucleotide of any of embodiments 17-28, the vector of any of embodiments 29-31, or the engineered cell of any of embodiments 32-37.
40. The composition of embodiment 39, further comprising a pharmaceutically acceptable excipient.
41. A method for identifying a T cell receptor (TCR) targeting a hematopoietically restricted minor histocompatibility antigen (miHA), the method comprising identifying a functional TCR that recognizes a hematopoietically-restricted miHA, among a plurality functional TCRs, wherein said plurality of functional TCRs are encoded by a plurality of functional TCR-encoding nucleic acid vectors generated by a high-throughput nucleic acid amplification and assembly method using nucleic acid obtained from a single T cell among a plurality of T cells; wherein said plurality of T cells is from a human female donor that is pregnant with or has been pregnant with a fetus with a mismatched or immunogenic hematopoietically restricted miHA.
42. A method for identifying a T cell receptor (TCR) targeting a hematopoietically restricted minor histocompatibility antigen (miHA), the method comprising:
(i) generating a plurality of functional TCR-encoding nucleic acid vectors by a high-throughput nucleic acid amplification and assembly method using nucleic acid obtained from a single T cell among a plurality of T cells; wherein said T cell is from a human female donor that is pregnant with or has been pregnant with a fetus with a mismatched or immunogenic hematopoietically restricted miHA; and
(ii) identifying a functional TCR that recognizes a hematopoietically-restricted miHA, among a plurality functional TCRs encoded by the plurality of functional TCR-encoding nucleic acid vectors.
43. The method of embodiment 41 or 42, wherein the hematopoietically restricted miHA is a minor histocompatibility antigen HA-1.
44. The method of any of embodiments 41-43, wherein the identified functional TCR recognizes a peptide epitope of a minor histocompatibility antigen HA-1 in the context of an MHC molecule.
45. The method of embodiment 44, wherein the MHC molecule is a human leukocyte antigens (HLA)-A molecule.
46. The method of embodiment 45, wherein the HLA-A molecule is of serotype HLA-A*02:01.
47. The method of embodiment 45, wherein the HLA-A molecule is of serotype HLA-A*02:06.
48. The method of any of embodiments 44-47, wherein the peptide epitope of HA-1 is set forth in SEQ ID NO:354.
49. The method of any of embodiments 41-48, wherein the T cell from the human female donor is cultured under conditions for cell expansion of the T cell prior to the generating of the plurality of functional TCR-encoding nucleic acid vectors.
50. The method of any of embodiments 41-48, wherein the T cell from the human female donor is not cultured under conditions for cell expansion of the T cell prior to the generating of the plurality of functional TCR-encoding nucleic acid vectors.
51. The method of any of embodiments 41-50, wherein the high-throughput nucleic acid amplification and assembly method comprises:

(1) amplifying a first amplification product and a second amplification product from complementary DNA (cDNA) generated from RNA obtained from the single T cell among the plurality of T cells sorted into each of a plurality of separate locations of a device, wherein:
said first amplification product comprises a nucleotide sequence encoding a full-length variable alpha (Vα) region or a full-length variable gamma (Vγ) region of a TCR, and said second amplification product comprises a nucleotide sequence encoding a full-length variable beta (Vβ) region or a full-length variable delta (Vδ) region of a TCR; and
(2) assembling said first amplification product and said second amplification product from each of said plurality of separate locations into a nucleic acid vector to obtain an assembled nucleic acid vector comprising a nucleotide sequence encoding a functional TCR for each of said plurality of separate locations; and
said functional TCR comprises (i) a full-length Vα region and a full-length Vβ region from said single T cell or (ii) a full-length Vγ region and a full-length Vδ region from said single T cell.
52. An engineered cell, comprising the TCR identified by the method of any of embodiments 41-51.
53. A composition comprising the engineered cell of embodiment 52.
54. The composition of embodiment 53, further comprising a pharmaceutically acceptable excipient.
55. A method of treatment, the method comprising administering the TCR or antigen-binding fragment thereof of any of embodiments 1-16, the polynucleotide of any of embodiments 17-28, the vector of any of embodiments 29-31, the engineered cell of any of embodiments 32-37 or 52, or the composition of any of embodiments 39, 40, 53 and 54, to a subject having a disease or a disorder.
56. The method of embodiment 55, wherein the subject is eligible for or is to receive an allogeneic hematopoietic stem cell transplantation (HSCT).
57. The method of embodiment 55 or 56, wherein the subject has or has been diagnosed with a malignant hematologic disorder.
58. The method of any of embodiments 55-57, wherein the subject has or has been diagnosed with acute myeloid leukemia (AML), myelodysplastic syndrome (MDS), or acute lymphoblastic leukemia (ALL).
59. The method of any of embodiments 55-58, wherein the administering step induces or enhances cells death of cells associated with the malignant hematologic disorder, or induces or enhances a graft versus leukemia effect (GVL) in the subject.
60. The TCR or antigen-binding fragment thereof of any of embodiments 1-16, the polynucleotide of any of embodiments 17-28, the vector of any of embodiments 29-31, the engineered cell of any of embodiments 32-37 or 52, or the composition of any of embodiments 39, 40, 53 and 54, for use in the treatment of a disease or a disorder in a subject.
61. Use of the TCR or antigen-binding fragment thereof of any of embodiments 1-16, the polynucleotide of any of embodiments 17-28, the vector of any of embodiments 29-31, the engineered cell of any of embodiments 32-37 or 52, or the composition of any of embodiments 39, 40, 53 and 54, in the manufacture of a medicament for the treatment of a disease or a disorder in a subject.
62. Use of the TCR or antigen-binding fragment thereof of any of embodiments 1-16, the polynucleotide of any of embodiments 17-28, the vector of any of embodiments 29-31, the engineered cell of any of embodiments 32-37 or 52, or the composition of any of embodiments 39, 40, 53 and 54, for the treatment of a disease or a disorder in a subject.

### VIII. EXAMPLES

The following examples are included for illustrative purposes only and are not intended to limit the scope of the invention.

### Example 1: Donor Selection and Candidate T Cell Receptor Screening

T cells expressing TCRs that can target hematopoietically-restricted miHA HA-1 were obtained from parous women, and screened for binding to particular HA-1 peptide variants.

### A. Donor Criteria

T cells expressing a TCR that can target an immunogenic alleles of a miHA were obtained from parous women, as they may have been naturally immunized to paternal allele of the miHA from the fetus during pregnancy and at the time of delivery. Donor T cells were isolated and screened for their ability to specifically target an immunogenic allele of the hematopoietically-restricted miHA HA-1.

Blood of multiparous volunteers was analyzed using whole exome sequencing to determine their HLA repertoire and polymorphisms encoding the HLA-A*0201 restricted HA-1 peptide (VLHDDLLEA; SEQ ID NO:354) or the non-immunogenic “R” variant (VLRDDLLEA; SEQ ID NO:355). Volunteers having an appropriate HLA type, i.e., an HLA-A*02:01 restricted HA-1 R/R phenotype, were selected as potential donors. Donor candidacy was further assessed by genotyping the volunteer's child or the father of said child to determine the presence of HA-1 H/H or HA-1 H/R. Detection of either “H” phenotype from the child or the father was indicative of potential exposure to the mother's immune system during pregnancy/childbirth. PBMCs were drawn from donors who met the described criteria.

### B. PBMC Screening to Identify HA-1-Targeting T Cells

Collected PBMCs were either directly screened for the presence of HA-1-specific T cells or first expanded in vitro. Using cells from the same donor, unexpanded and expanded cultures were compared to inform future screening strategies.

### 1. Direct Sample Screening

Donor cells were stained with an APC A*0201/HA-1 fluorescent labeled dextramer then sorted using fluorescence-activated cell sorting (FACS). From 5×10⁷ PBMCs, 47 single CD8+ T cells stained positive for HA-1. The HA-1 reactive cells were then added to a 384-well plate for further processing.

### 2. Expanded Culture Screening

3.4×10⁷ PBMCs from the same donor were expanded in vitro using either the naked peptide or the peptide in the presence of donor B cells. In the first approach, resident antigen presenting cells (APCs) in the PBMC sample were stimulated by adding 10 μg/ml of the HA-1 “H” peptide. The cells were then cultured in the presence of cytokines for 10 days. In the second approach, CD19+B cells (B-APCs) were similarly co-cultured for 10 days at a 1:10 B-APC/PBMC ratio, again in the presence of cytokines.

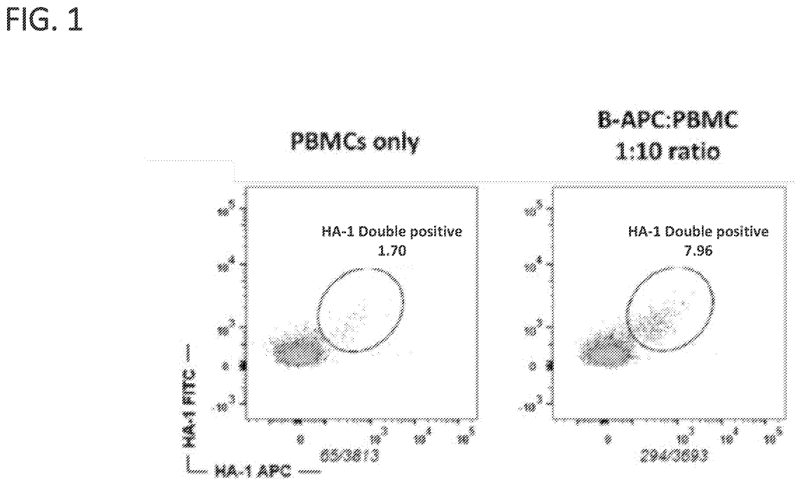

At day 10, samples from both cultures were analyzed by FACS for expansion. Cells were stained with an irrelevant A*0201 dextramer for counter-selection. Two HA-1/A*0201 dextramers, one labeled with APC, the other with FITC, were used to detect desirably reactive TCRs. FIG. 1 shows the detection of double positive cells in both expanded cell cultures, indicating the potential presence of specific HA-1 TCRs. 704 total CD8+ T cells were added to 384-well plates for amplification, cloning and assessment based on a high-throughput TCR amplification and assessment methods as described in Example 2 below.

### Example 2: High-Throughput TCR Chain Amplification and Expression

Sorted T cells that potentially express TCRs specific for an immunogenic allele of HA-1 obtained from multiparous women, as described in Example 1 above, were assessed using a high-throughput TCR cloning and identification method.

### A. TRAV/TRBV Amplification

Cells in 384-well plates were stained with a FITC/APC HA-1 dextramer prior to analysis. Cells that stained double-positive for FITC/APC HA-1 dextramer were single cell sorted using FACS.

The T cell receptor alpha variable (TRAV) and T cell receptor beta variable (TRBV) chains of the sorted cells were amplified using a high throughput method, generally as described in WO 2018/102473. TRAV was efficiently amplified (concentrations of more than 5 ng/μl DNA) in 683 wells (97%), and TRBV in 632 wells (90%), resulting in an overall amplification of 625 TCR alpha/beta pairs (89%). Amplified TRAV/TRBV from each of the sorted cells were assembled into a lentivirus plasmid vector, resulting in the generation of 701/704 positive bacterial cultures, an indication of proper plasmid assembly.

### B. Cell Surface Expression and Target Binding

Plasmids were extracted using an automated platform, and were transfected at once using robotics into an HEK293 cell line engineered to permit surface TCR expression by cells expression of human CD3γ, CD3δ, CD3ε, CD3ζ (polyCD3) and human CD8 α/β.

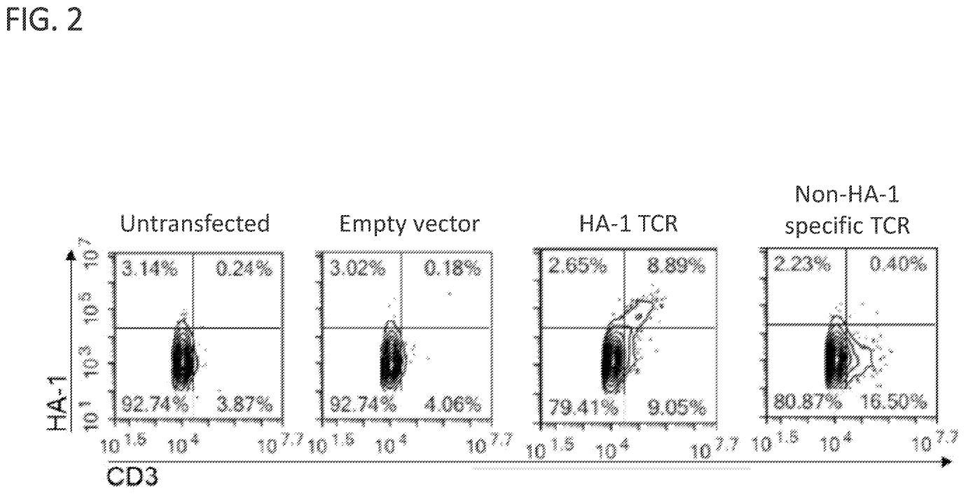

After 24 hours, cells were stained with both an anti-CD3 antibody, to determine surface TCR expression, and an HA-1 dextramer to screen for specific T cell receptors. FIG. 2 shows the FACS analysis of the controls. A model HA-1 receptor identified from non-expanded screen was used as a positive control showing both CD3 and HA-1 dextramer staining. An ACC-1 TCR derived from a separate screen was used as a negative control, exhibiting surface TCR expression by the CD3 staining but no specific binding to the HA-1 dextramer.

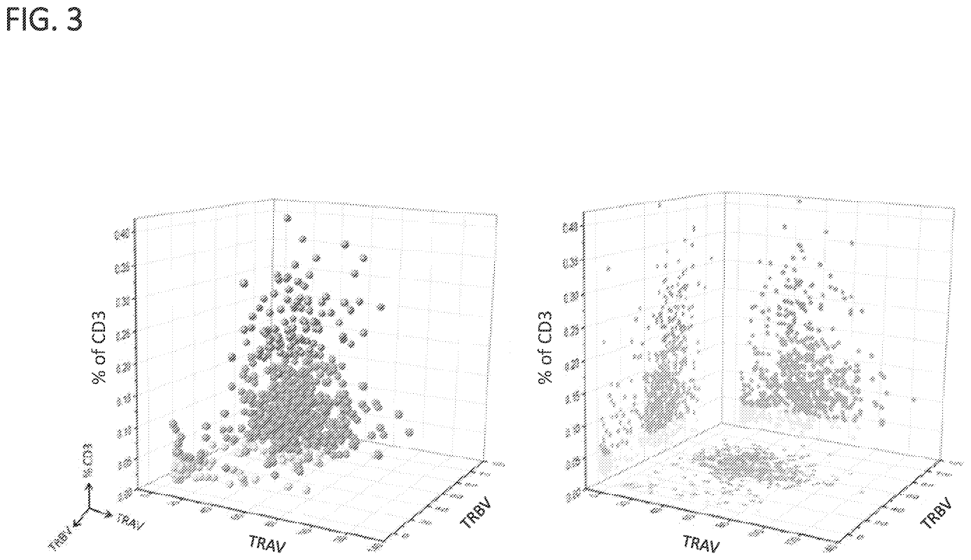

As depicted in FIG. 3, FACS analysis after the transient expression of 701 clones into HEK293 cells showed that there were 103 clones having higher surface TCR expression than the positive control (blue), 174 clones below the negative control (yellow), and 427 that fell in between (green). Overall, productive TCR expression was observed in 75% (504/704) of clones from expanded cells.

For T cells that stained positive for HA-1 obtained from direct sample screening (Example 1.B.1 above), the sequences encoding the TCR were assembled into a lentivirus vector that co-expresses a red fluorescent marker (mCherry) as a transfection efficiency control. The plasmids were directly transiently transfected into the engineered HEK293 cells permissive for surface expression of TCRs.

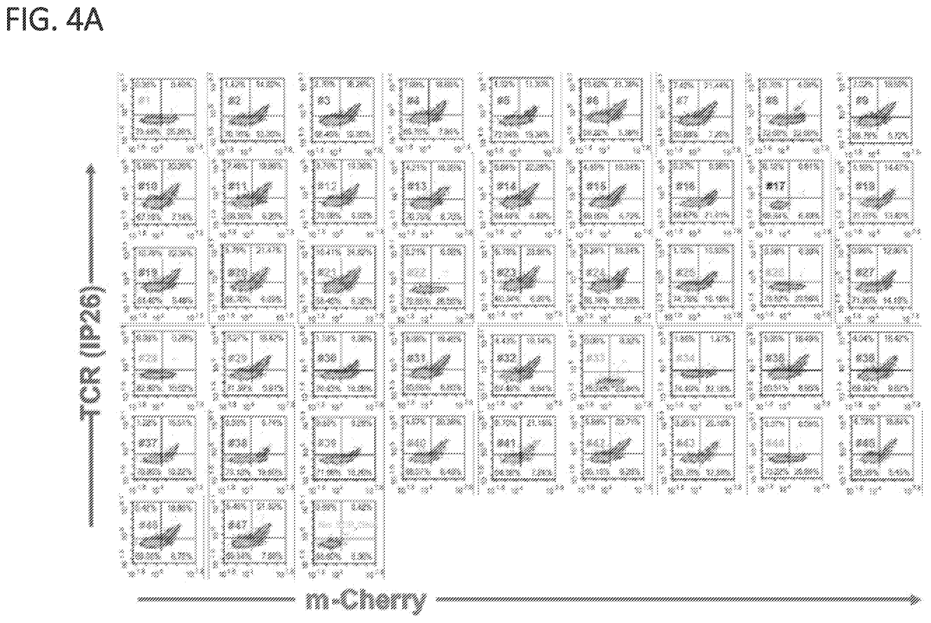

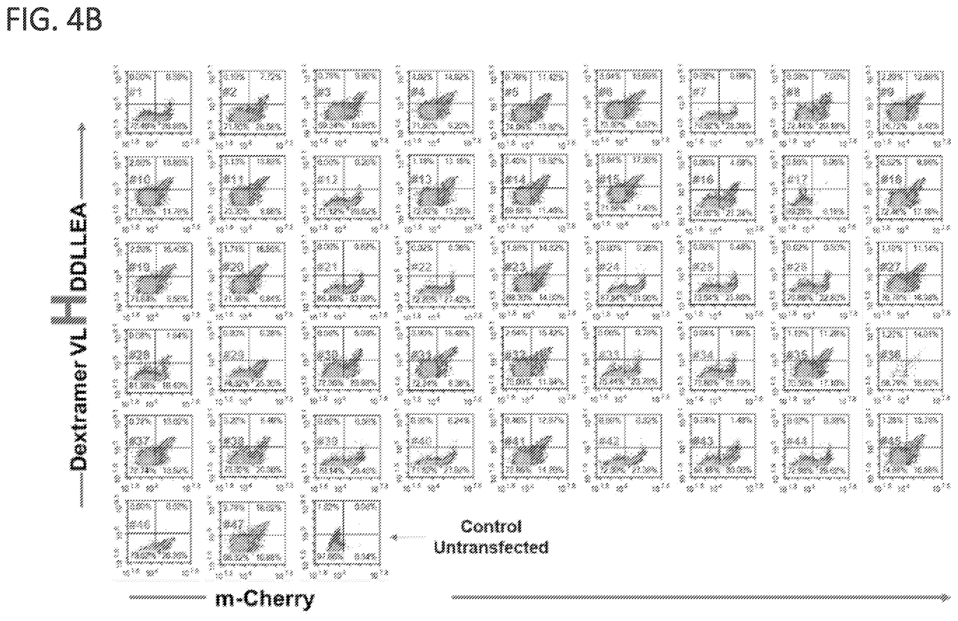

After overnight incubation, cells were assessed by FACS. A high transfection efficiency was observed in HEK cells transfected with TCRs from unexpanded cells. As shown in FIG. 4A, 40 out of 47 (85%) transfections resulted in TCR expression as determined by staining with a monoclonal antibody IP26 that detects TCR surface expression. As shown in FIG. 4B, 30/40 stained positive with the HA-1 dextramer.

### C. TRBV Sequencing

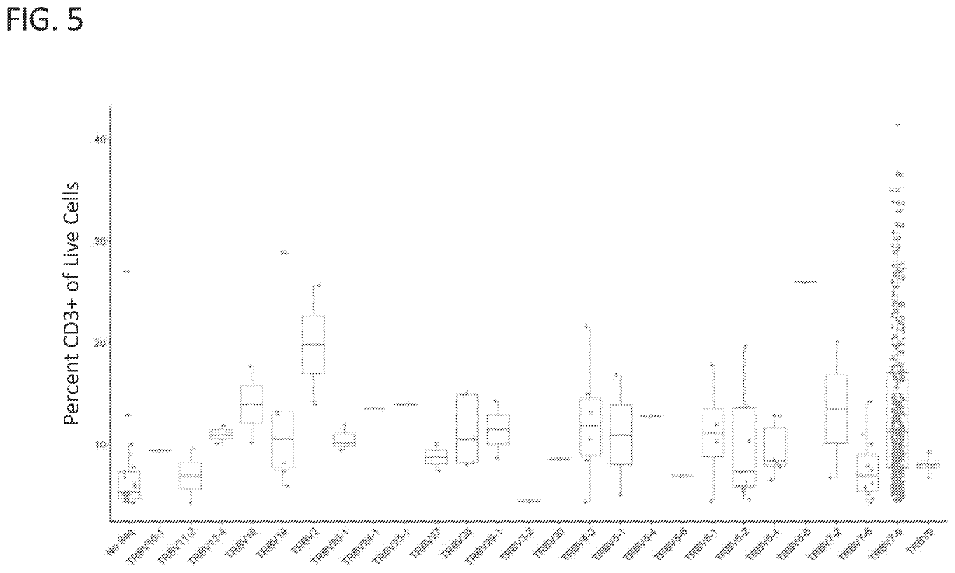

TRBVs were sequenced in parallel with the expression screen described above to assess expansion and clonality. From the 704 originally selected expanded cells, 531 readable sequences were generated. FIG. 5 shows that the TRBV7-9 gene was present in the majority of isolated TCR beta chains. The data was similar to TRBV sequencing results of unexpanded cells.

This result indicates significant enrichment via successful expansion of specific HA-1 T cells. Overall, 26 different TRBV genes were detected. There was no association between TRBV chain type and TCR expression.

### D. TCR Attributes of Unexpanded and Expanded Donor PBMCs

The TCRs from T cells from unexpanded and expanded screens as described in Example 1 were analyzed to assess clonal distribution and sensitivity of the high-throughput TCR identification method.

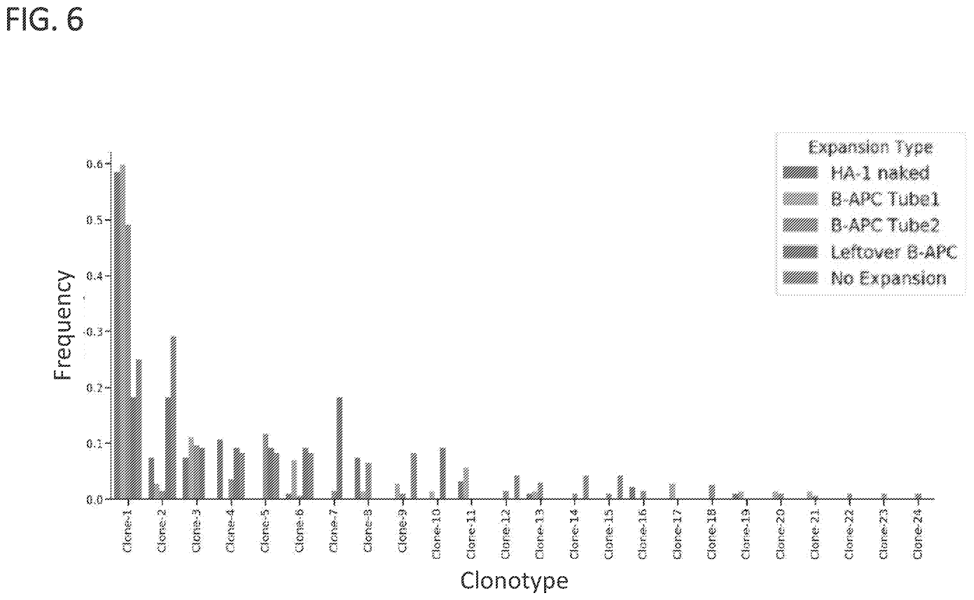

FIG. 6 shows the distribution of non-singleton clones from the expanded and non-expanded T cells as described in Example 1. Sequencing of 30 clones that stained positive with the HA-1 dextramer from the direct sample screening showed 15 distinct clones, based on their unique TRAV and TRBV sequences. As shown in FIG. 6, 9 out of the 15 most prevalent TCRs in the expanded screen were identified without any expansion (purple bars). With regard to the expanded cells, only 4 clones were present in all three expanded populations. One particular clone was highly prevalent in all three expanded populations and in the non-expanded population, indicating that this particular clone was also the most abundant in the non-expanded population.

These results demonstrate the ability of the high-throughput screening methods to identify candidate TCRs with desired features, even when they are sparsely represented, as in an unexpanded sample. The results support a substantial advantage of the described high-throughput screening method to identify TCRs of interest without the requirement of expansion, particularly in contexts in which screening is time sensitive, processing can jeopardize sample integrity, or when expansions are not guaranteed, such as for tumor infiltrating lymphocytes. Surprisingly, functional TCRs were cloned and expressed even in samples with low levels of TRAV/TRBV amplification. These results show the efficiency and the utility of the cloning methods, even when low levels of amplification products are available.

### Example 3: Exemplary Identified TCRs

The Examples describe the successful isolation, cloning, screening, identification, sequence determination and characterization of minor histocompatibility HA-1-specific TCRs. The TCRs were obtained from parous women and screened using a high-throughput method to obtain full length TCRs. From one population of screened T cells, 30 out of 47 isolated cells were positive for HA-1 specific binding and represented 15 unique TCR clones. Three (3) TCRs exhibited an EC₅₀) below 200 nM against the immunogenic HA-1 peptide, and many TCRs exhibited a more than 100-fold specificity towards the immunogenic H peptide of HA-1, compared to the non-immunogenic R peptide. Exemplary TCRs also recognized naturally expressed peptides in H:R or H:H cells, in a gene dose-dependent manner. Primary T cells expressing an exemplary HA-1 specific TCR exhibited target cell killing activity against cells loaded with the immunogenic H peptide, but not the non-immunogenic R peptide. No indication of alloreactivity was observed.

Table E1 lists the sequence identifiers (SEQ ID NO:) for amino acid (aa) or nucleotide (nt) sequences for exemplary HA-1 specific TCRs that were isolated, assessed, and sequenced using methods described above. The table also lists the sequence identifier (SEQ ID NO:) corresponding to an exemplary full-length, including the constant domains, amino acid sequence containing the alpha and beta chain sequences of each respective TCR, separated by a sequence encoding a ribosome-skip P2A sequence (P2A linker set forth in SEQ ID NO: 352 encoded by the nucleotides set forth in SEQ ID NO: 351) (designated “alpha-P2A-beta”).

TABLE E1
Amino Acid and Nucleotide Sequences of HA-1 Specific TCRs

| | | | | | | | | | | | Full | |
|---|---|---|---|---|---|---|---|---|---|---|---|---|
| | | | | | | | | | | | length | |
| | Alpha variable | | | | | Beta variable | | | | | alpha- | |
| | | | CDR- | CDR- | CDR- | | | CDR- | CDR- | CDR- | P2A-beta | |
| TCR | nt | aa | 1 (aa) | 2 (aa) | 3 (aa) | nt | aa | 1 (aa) | 2 (aa) | 3 (aa) | nt | aa |
| TCR A | 7 | 4 | 1 | 2 | 3 | 15 | 12 | 9 | 10 | 11 | 17 | 18 |
| TCR B | 25 | 22 | 19 | 20 | 21 | 31 | 28 | 9 | 10 | 27 | 33 | 34 |
| TCR C | 41 | 38 | 35 | 36 | 37 | 47 | 44 | 9 | 10 | 43 | 49 | 50 |
| TCR D | 55 | 52 | 1 | 2 | 51 | 61 | 58 | 9 | 10 | 57 | 63 | 64 |
| TCR E | 69 | 66 | 1 | 2 | 65 | 72 | 58 | 9 | 10 | 57 | 74 | 75 |
| TCR F | 82 | 79 | 76 | 77 | 78 | 88 | 85 | 9 | 10 | 84 | 90 | 91 |
| TCR G | 96 | 93 | 19 | 20 | 92 | 102 | 99 | 9 | 10 | 98 | 104 | 105 |
| TCR H | 110 | 107 | 76 | 77 | 106 | 116 | 113 | 9 | 10 | 112 | 118 | 119 |
| TCR I | 124 | 121 | 35 | 36 | 120 | 130 | 127 | 9 | 10 | 126 | 132 | 133 |
| TCR J | 140 | 137 | 134 | 135 | 136 | 146 | 143 | 9 | 10 | 142 | 148 | 149 |
| TCR K | 156 | 153 | 150 | 151 | 152 | 162 | 159 | 9 | 10 | 158 | 164 | 165 |
| TCR L | 170 | 167 | 35 | 36 | 166 | 176 | 173 | 9 | 10 | 172 | 178 | 179 |
| TCR M | 184 | 181 | 35 | 36 | 180 | 190 | 187 | 9 | 10 | 186 | 192 | 193 |
| TCR N | 198 | 195 | 76 | 77 | 194 | 204 | 201 | 9 | 10 | 200 | 206 | 207 |
| TCR O | 212 | 209 | 76 | 77 | 208 | 218 | 215 | 9 | 10 | 214 | 220 | 221 |
| TCR P | 228 | 225 | 222 | 223 | 224 | 234 | 231 | 9 | 10 | 230 | 236 | 237 |
| TCR Q | 242 | 239 | 76 | 77 | 238 | 248 | 245 | 9 | 10 | 244 | 250 | 251 |
| TCR R | 256 | 253 | 19 | 20 | 252 | 262 | 259 | 9 | 10 | 258 | 264 | 265 |
| TCR S | 272 | 269 | 266 | 267 | 268 | 274 | 159 | 9 | 10 | 158 | 276 | 277 |
| TCR T | 282 | 279 | 19 | 20 | 278 | 288 | 285 | 9 | 10 | 284 | 290 | 291 |
| TCR U | | 360 | | | 359 | | 364 | 9 | 10 | 363 | 368 | 367 |
| TCR V | | 370 | | | 369 | | 374 | 9 | 10 | 373 | 378 | 377 |
| TCR W | | 380 | | | 379 | | 384 | 9 | 10 | 383 | 388 | 387 |
| TCR X | | 390 | | | 389 | | 394 | 9 | 10 | 393 | 398 | 397 |
| TCR Y | | 400 | | | 399 | | 404 | 9 | 10 | 403 | 408 | 407 |
| TCR Z | | 410 | | | 409 | | 414 | 9 | 10 | 413 | 418 | 417 |
| TCR AA | | 420 | | | 419 | | 424 | 9 | 10 | 423 | 428 | 427 |
| TCR AB | | 430 | | | 429 | | 434 | 9 | 10 | 433 | 438 | 437 |
| TCR AC | | 440 | | | 439 | | 444 | 9 | 10 | 443 | 448 | 447 |
| TCR AD | | 450 | | | 449 | | 454 | 9 | 10 | 453 | 458 | 457 |
| TCR AJ | | 481 | | | 480 | | 485 | | | 484 | 489 | 488 |

### Example 4: Characterization of Exemplary HA-1 Specific TCRs

As described herein, minor histocompatibility antigens (miHAs) relatively restricted to hematopoietic cells are ideal targets for adoptive T cell immunotherapy in the context of allogeneic stem cell transplantation (alloSCT), as T cells that target them can mediate graft-versus-leukemia and promote engraftment with a low risk for graft-vs-host disease. Multiple exemplary TCRs reactive against the hematopoietic cell-restricted miHA HA-1, isolated from a parous woman who was naturally immunized to HA-1 through pregnancy and identified using the high-throughput screening method generally as described in Examples 1-3 above, were characterized.

In summary, an HLA-A*02:01 woman homozygous for the non-immunogenic HA-1 (R/R) allele who delivered three children with an HA-1 heterozygous (H/R) HLA-A*02:01 father was identified. TCRs were cloned from single-cell-sorted HA-1 dextramer+(dex^{HA-1+}) CD8⁺ T cells from unstimulated peripheral blood mononuclear cells (PBMCs) and subsequently from the CD8⁺ T cells co-cultured for one week with HA-1 peptide-pulsed antigen-presenting cells (APCs). TCRs were re-expressed in reporter cells using lentivirus vectors and analyzed for dextramer binding and CD69 upregulation after culture with HA-1(H) peptide-pulsed APCs. Cloned TCRs were sequenced to characterize TCR diversity.

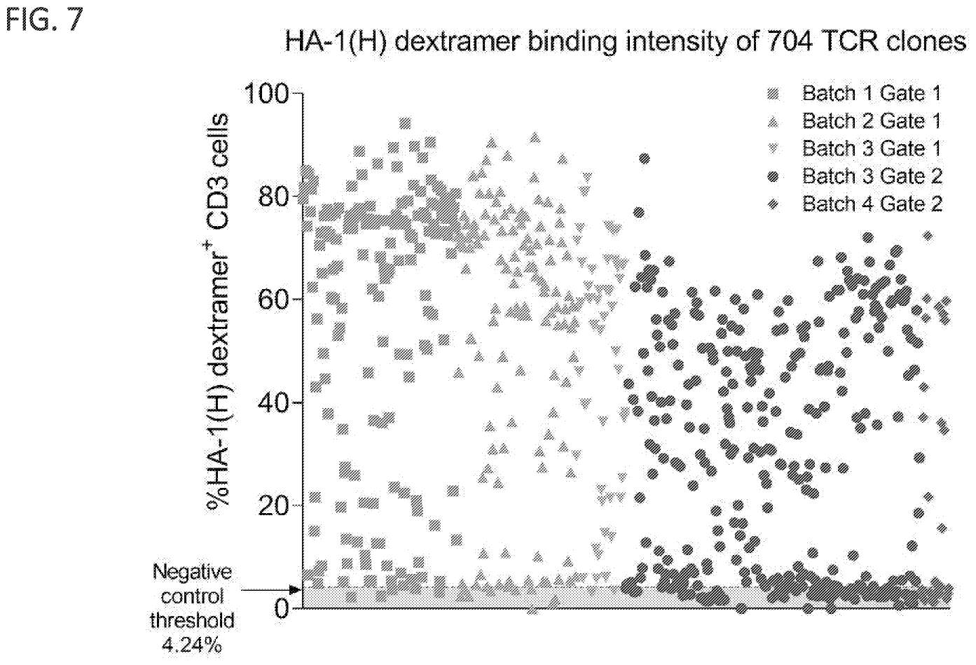

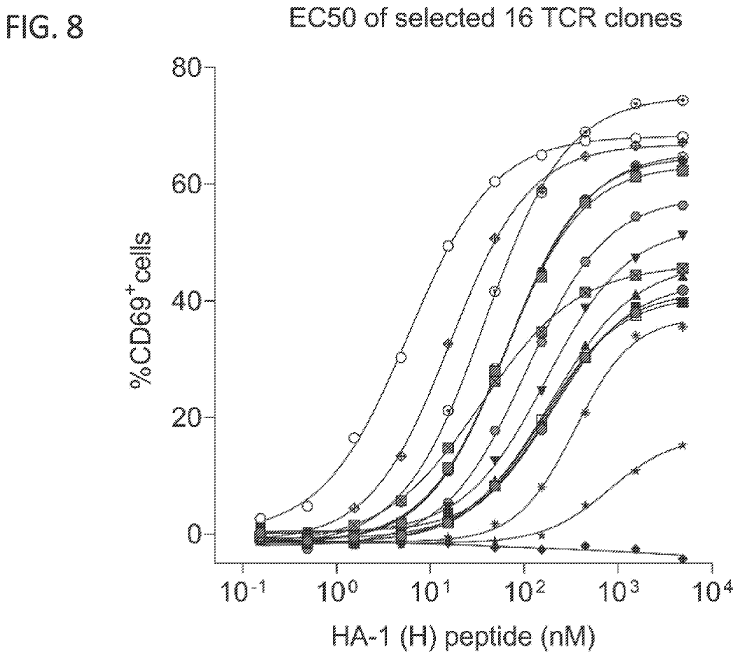

Sixteen (16) unique HA-1-reactive TCRs from 48 sorted dex^{HA-1+} CD8⁺ T cells from unstimulated PBMCs. 704 additional TCRs were cloned from HA-1 peptide-stimulated CD8 cells. When re-expressed, 440 bound HA-1(H) dextramer with various intensities, as shown in FIG. 7. HA-1 specific TCRs exhibited a broad range of EC₅₀s, although they all used TRBV7-9, as shown in FIG. 8. Exemplary HA-1 TCR clones, when re-expressed in primary CD8+ T cells, killed HA-1+ target cells.

The results are consistent with the isolation of various TCRs exhibiting a wide range of affinities against a single allopeptide/HLA complex (VLHDDLLEA/HLA-A*02:01). The results support the utility of the described approaches to clone and characterize TCRs targeting hematopoietically restricted miHAs, for adoptive T cell therapy in alloSCT.

### Example 5: Binding Specificity of Exemplary HA-1 Specific TCR

Binding specificity of the HA-1 specific TCRs was determined using dextramers complexed with immunogenic or non-immunogenic HA-1 peptides.

HEK293 suspension cells engineered to express human CD3γ, CD3δ, CD3ε, CD3ζ (polyCD3) and human CD8 α/β. CD3 and CD8 were cloned from pooled PBMCs from two healthy blood donors and introduced into HEK293 cells using separate expression plasmids. Suspension HEK293-CD3-CD8 cells were transiently transfected with plasmid DNA containing an anti-HA-1 TCR and the fluorescent protein mCherry, as a transfection efficiency control.

Twenty-four hours after transfection, cells were stained with an amine-reactive viability dye (violet 510 Ghost dye), anti-CD3 and the immunogenic HA-1 “H” peptide dextramer or non-immunogenic HA-1 “R” peptide dextramer. Cells were acquired and analyzed by flow cytometry.

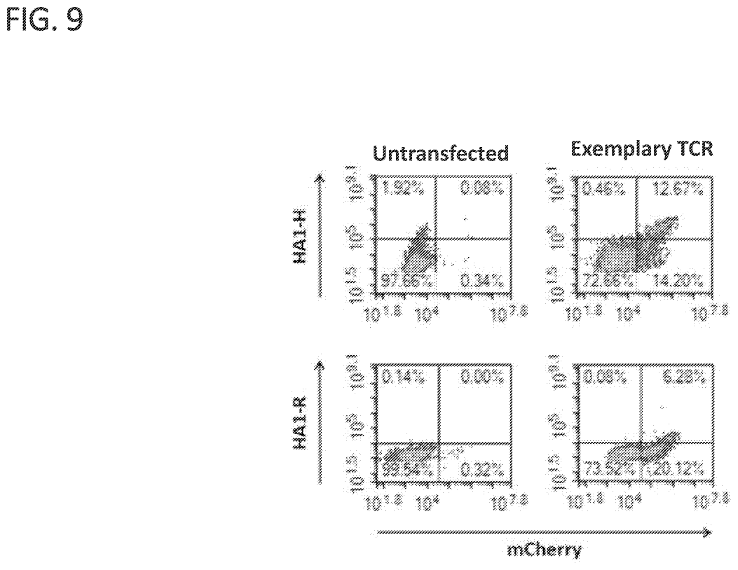

Double positive fluorescence of mCherry and specific binding of HLA-dextramers complexed with the HA-1 “H” peptide indicated a functional and desirably reactive TCR. FIG. 9 shows the transfection efficiency and enhanced “H” peptide binding of an exemplary TCR. The results demonstrate the ability of the high-throughput screening methods to identify candidate TCRs that can specifically distinguish an immunogenic allele of HA-1 (from transplant cell donor) from a non-immunogenic allele (from transplant recipient).

### Example 6: In Vitro Evaluation of Engineered T Cell Reactivity to Hematopoietically Restricted Minor Histocompatibility Antigen HA-1

TCRs isolated and identified as described in Examples 1-3 above were recombinantly expressed in a cell line, further characterized and assessed for function, including cytokine secretion, T cell activation and binding specificity.

### A. Cytokine Secretion

To assess the function of anti-HA-1 TCR-bearing T cells, the secretion of IL-2, an activation-induced cytokine, in response to co-culture with peptide-loaded APCs was investigated using an enzyme-linked immune absorbent spot (ELISpot) assay.

A Jurkat J.RT3-T3.5-CD8 T-cell stable cell line was engineered to express human CD8α/β. CD8 was cloned from mixed PBMCs from two healthy donors. The cell line was then transduced with a lentivirus (pLVX-Puro, Clontech Laboratories, Inc.) expressing the various HA-1 TCR identified as described above.

The transduced Jurkat T cells were co-incubated overnight with A*0201 HLA Lymphoblastoid Cell Lines (LCLs) that are used for presentation into various MHC molecules and serve as APCs, at a 1:1 effector to target ratio (E/T) in the presence or absence of the immunogenic HA-1 H peptide (VLHDDLLEA). Analysis of cell mixtures using ELISpot was performed according to the manufacturer's instructions (Human IL-2 ELISpotbasic, MabTech) to assess the ability of the Jurkat T cells to secrete IL-2 in the presence of APCs presenting the target HA-1 peptide.

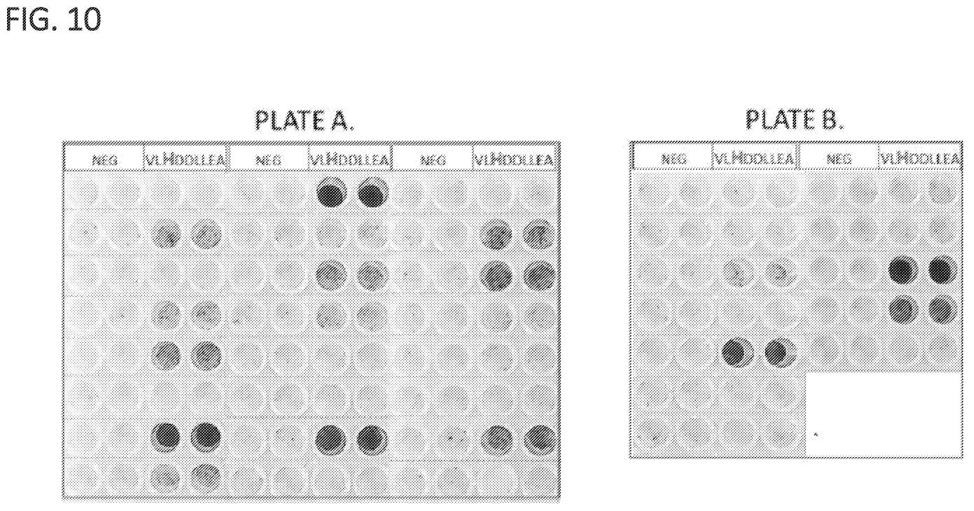

FIG. 10 shows exemplary results of the IL-2 secretion for various TCRs. The number and intensity of spots indicated the level of IL-2 secretion. The results confirmed target-specific T cell activation of the TCR expressing cells, and showed that receptors with different activities were isolated.

For an exemplary HA-1 specific TCR, Jurkat T cells expressing the HA-1 TCR were co-cultured with T2 lymphoblast cells pulsed with HA-1 “H” or HA-1 “R” peptide. Equal numbers of Jurkat T cells and T2 cells loaded with increasing concentrations (0.1-31.6 ng/ml) of either HA-1 peptide were co-cultured for 16 hours. IL-2 secretion was assessed by an ELISpot as above. Jurkat T cells without T2 APCs and T2 APCs without Jurkat T cells served as negative controls.

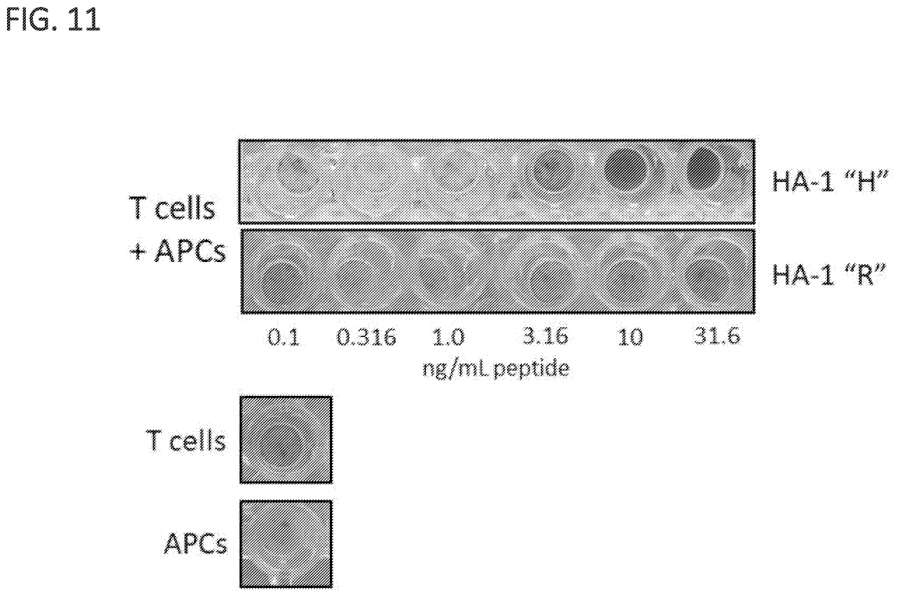

FIG. 11 shows the results of IL-2 secretion based on presentation of allele-specific HA-1 peptide, for cells expressing an exemplary TCR. As shown in FIG. 11, the presentation of increasing amounts of immunogenic HA-1 “H” peptide resulted in increased IL-2 secretion of T cells expressing the exemplary HA-1 TCR. Interestingly, across the same tested peptide concentrations, IL-2 secretion in response to the HA-1 “R” peptide was minimal. Observing IL-2 secretion from engineered HA-1 T cells in response to the target confirms target-specific functionality of the identified TCR.

### B. Assessment of Early Activation Marker CD69

Expression of CD69, a marker of T cell activation and function, was assessed following co-culture of Jurkat T cells expressing an HA-1 TCR with APCs loaded with HA-1 peptides.

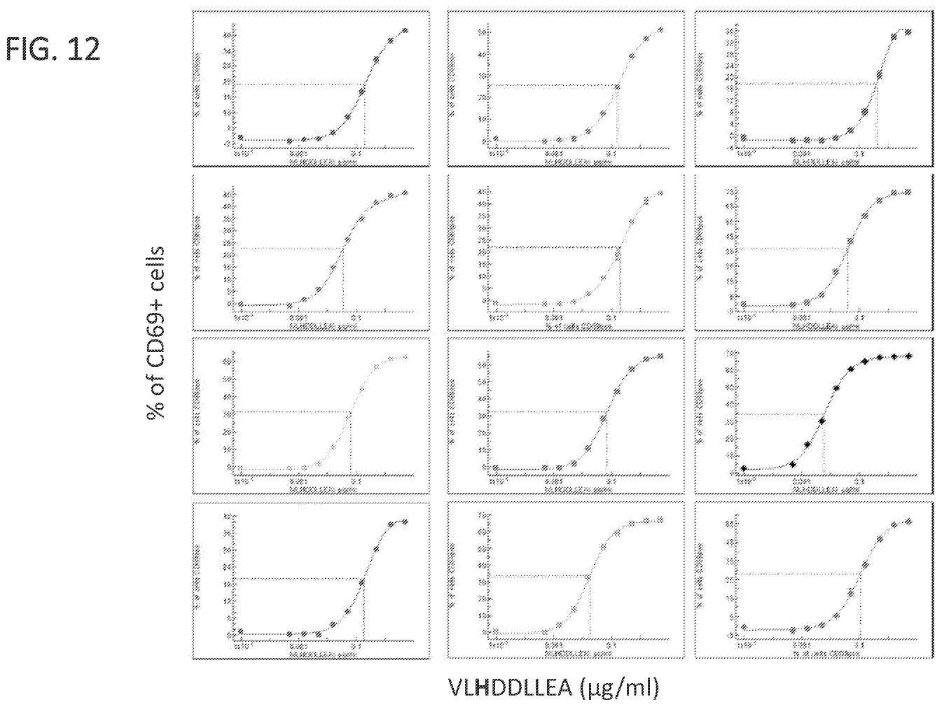

Jurkat J.RT3-T3.5-CD8 T cells expressing various anti-HA-1 TCRs were prepared as described above in Example 4.A. Jurkat cells transduced with anti-HA-1 TCRs were incubated overnight with A*0201 LCLs at a 1:1 E/T ratio in the presence of increasing concentrations of HA-1. FIG. 12 shows the percentage of CD69+ cells of twelve different TCR clones over a span of peptide concentrations. The EC₅₀ values were calculated based on XLfit (ID Business Solutions) on plots of the percentage of CD69+ cells (y-axis) vs. peptide concentration (logarithmic x-axis). Table E3 lists the determined EC₅₀ values for CD69 expression for the exemplary HA-1-specific TCRs. Several clones were found to exhibit an EC₅₀ at low double-digit nM range, indicating high affinity binding to LCLs loaded with cognate peptides. The results showed a broad range of EC₅₀ values, showing a surprising functional diversity of the identified TCRs despite all using the TRBV7-9 gene; except, TCR AJ, which used the TRBV5-5 gene.

TABLE E3
EC₅₀ for CD69 Expression in Exemplary HA-1 Specific TCRs

| | EC₅₀ (ng/mL) | EC₅₀ (nM) |
|---|---|---|
| TCR A | <10 | <10 |
| TCR B | 10-100 | 10-100 |
| TCR C | <10 | <10 |
| TCR D | <10 | <10 |
| TCR E | <10 | <10 |
| TCR F | <10 | <10 |
| TCR G | 10-100 | 10-100 |
| TCR H | 10-100 | 10-100 |
| TCR I | 10-100 | 10-100 |
| TCR J | 10-100 | 10-100 |
| TCR K | 10-100 | 10-100 |
| TCR L | 10-100 | 10-100 |
| TCR M | 10-100 | 10-100 |
| TCR N | 10-100 | 10-100 |
| TCR O | 10-100 | 10-100 |
| TCR P | 10-100 | 10-100 |
| TCR Q | 100-1000 | 100-1000 |
| TCR R | 100-1000 | 100-1000 |
| TCR S | 100-1000 | 100-1000 |
| TCR T | 100-1000 | 100-1000 |

TABLE E3-1
EC50 values for CD69 Expression
in Exemplary HA-1 Specific TCRs

| | EC₅₀ (ng/mL) |
|---|---|
| TCR U | 12.73 |
| TCR V | 124.51 |
| TCR W | 7.18 |
| TCR X | 23.55 |
| TCR Y | 10.84 |
| TCR Z | 1.5 |
| TCR AA | 1.1 |
| TCR AB | 40.2 |
| TCR AC | 0.9 |
| TCR AD | 4.9 |
| TCR AJ | 5.0 |

TABLE E3-1
EC50 values for CD69 Expression in Exemplary
engineered HA-1 Specific TCRs

| | EC₅₀ (ng/mL) |
|---|---|
| TCR AE | 2.77 |
| TCR AF | 2.49 |
| TCR AG | 2.27 |
| TCR AH | 3.4 |
| TCR AI | 9.2 |

For an exemplary TCR, the anti-HA-1 TCR-expressing T cells were co-cultured in duplicate at a 1:1 effector-to-target ratio with peptide-loaded T2 cells. After 16-24 hours of culture, cells are washed and stained with Ghost Dye, anti-CD3 and anti-CD69 and assessed by flow cytometry. Data were analyzed by XLFit. The estimated EC₅₀ of each of the five experimental runs was determined by assessing the ratio of CD69 positive Jurkat cells to total live cells.

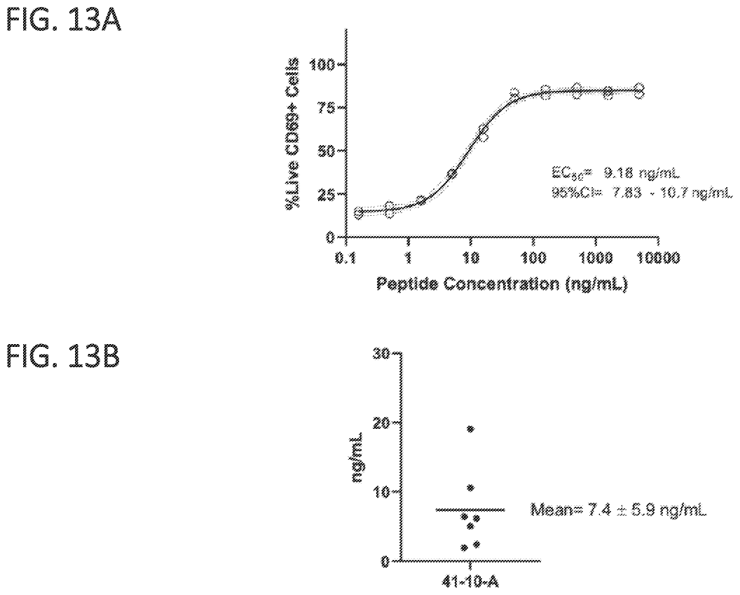

As shown in FIG. 13A (showing data from a single representative experiment), co-culturing T cells expressing an exemplary anti-HA-1 TCR with T2 cells loaded with increasing concentration of HA-1 “H” peptide (greater than a five log₁₀ concentration span), analyzed by flow cytometry for the percentage of cells positive for CD69, exhibits a corresponding increase of CD69 positive T cells. FIG. 13B is a graphical representation of the EC₅₀ determined for the HA-1 TCR from 6 separate experiments to show reproducibility and to determine a population mean with standard deviation. The mean EC₅₀, the average level of HA-1 “H” peptide necessary to produce CD69 upregulation in 50% of live T cells, was 7.4+5.9 ng/ml. Percentage of CD69+ cells did not increase in response to co-culture with T2 cells pulsed with the “R” peptide, suggesting specificity of the transduced T cells for the HA-1 “H” target.

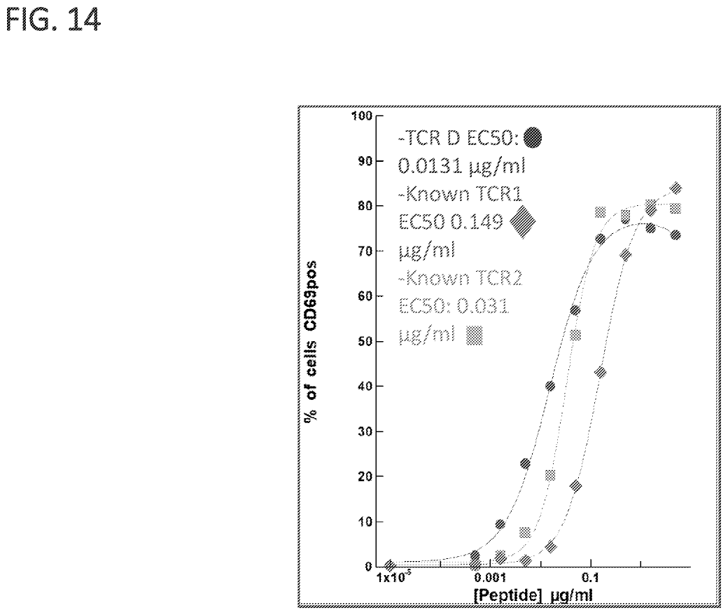

The EC₅₀ of CD69 expression for an exemplary HA-1 TCR isolated as described above, was compared to the EC₅₀ of known anti-HA-1 TCRs, reconstructed from published sequences. As shown in FIG. 14, an exemplary TCR identified using the methods described in Examples 1-3 above was found to have a three- to eleven-fold lower EC₅₀ for CD69 activation, which indicates a comparable, if not greater, potency in a reconstructed CD69 activation assay.

### C. Assessment of T Cell Receptor Specificity

Specificity of the candidate HA-1 targeting TCRs against the HA-1 “H” versus “R” peptide presented by the restricting HLA, or potentially other HLA molecules, or other HLA molecules presenting other peptides, were assessed.

### 1. LCL and Target Specificity

The specificity against the immunogenic HA-1 “H” allele compared to the non-immunogenic “R” allele was assessed based on a CD69 expression assay using various LCLs displaying different HA-1 alleles.

Jurkat J.RT3-T3.5-CD8 T cells expressing the various anti-HA-1 TCR were co-cultured for 16 hours at a 1:1 effector to target ratio with HLA-A*02:01 restricted LCLs that displayed various HA-1 haplotypes (Astarte Biologics). Fifteen different LCLs, each characterized by the presence or absence of the HA-1 “H” peptide, were used in the study. Following co-incubation, cells were stained with violet 510 Ghost dye and APC-conjugated anti-CD69. Cells were assessed by flow cytometry.

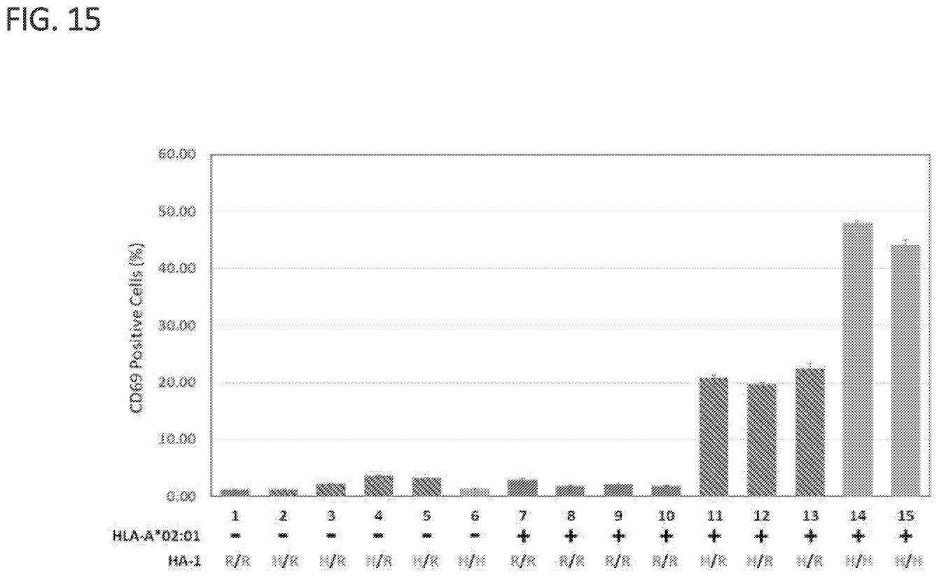

As shown in FIG. 15, T cell lines expressing an exemplary anti-HA-1 TCR only resulted in CD69 activation when co-cultured with LCLs that both express HLA-A*02:01 and were loaded with the immunogenic allele of HA-1, indicating that the anti-HA-1 TCR recognizes HA-1 that is naturally processed by a target cell. Minimal non-specific reactivity was observed, even in response to HA-1 “H” peptides presented by non-HLA-A*02:01 LCLs or HA-1 “R” peptides presented by HLA-A*02:01 LCLs. In addition, a gene dosage effect was observed, showing approximately a two-fold higher activation by LCLs that were H/H homozygous compared to H/R heterozygous LCLs. The results show specific activation of exemplary anti-HA-1 TCR expressing cells.

### 2. Alloreactivity

Candidate TCRs were screened against a panel of HLA-typed LCLs to assess possible alloreactivity. Table E4 lists an exemplary panel with individual HLAs. Using a method similar to the CD69 activation assay described in Example 4.B above, HA-1 TCRs were assessed for alloreactivity against the panel of HLA Class I and Class II molecules shown in Table E4. None of the analyzed TCRs exhibited alloreactivity.

TABLE E4
HLA Locus Class I and Class II Panel

| HLA locus CLASS I | | | | HLA locus CLASS II | | | | | | | |
|---|---|---|---|---|---|---|---|---|---|---|---|
| A | B | Bw | C | DRB1 | DRB3 | DRB4 | DRB5 | DQB1 | DQA1 | DPB1 | DPA1 |

| | | | | | | | | |
|---|---|---|---|---|---|---|---|---|
| A*0101 | B*0702 | C*0202 | *0103 | *0101 | *01AC | *0201 | *0301 | *0101 |
| A*0201 | B*0801 | C*03 | *03 | *0202 | | *0301 | *0501 | *0201 |
| A*0301 | B*1501 | C*04 | *0401 | | | *0304 | *05EF | *0401 |
| A*1101 | B*18 | C*0501 | *0404 | | | | *05BFK | *0402 |
| A*23 | B*35 | C*0602 | *0701 | | | | | |
| A*2402 | B*37 | C*0701 | *08 | | | | | |
| A*30 | B*4002 | C*15 | *11 | | | | | |
| A*3101 | B*4402 | | *12 | | | | | |
| A*34 | B*45 | | *1301 | | | | | |
| A*68 | B*5101 | | *14 | | | | | |
| | B*5701 | | *1501 | | | | | |
| | | | *21 | | | | | |

The results showed that T cells expressing various TCRs targeting HA-1 isolated and identified as described in Examples 1-3 conferred target-specific T cell function, as shown by cytokine secretion and CD69 expression, in response to the presence of APCs presenting the immunogenic HA-1 peptide.

### Example 7: Target Cell Killing of Primary Cells Transduced with HA-1-Specific TCRs

To determine the potential for inducing a graft-versus-leukemia (GvL) effect, the anti-leukemia target cell killing activity of primary T cells transduced with an exemplary anti-HA-1 TCR was assessed.

### A. Expression of HA-1 Specific TCRs in Primary Cells

Primary human CD8+ T cells were enriched from PBMCs by negative selection. CD8+ T cells were then transduced with a lentivirus vector (pReceiver-LV230 expression vector, GeneCopoeia) containing one TCR among various anti-HA-1 TCRs and a puromycin resistance gene cassette. The transduced T cells were enriched for expression of the TCR using puromycin selection.

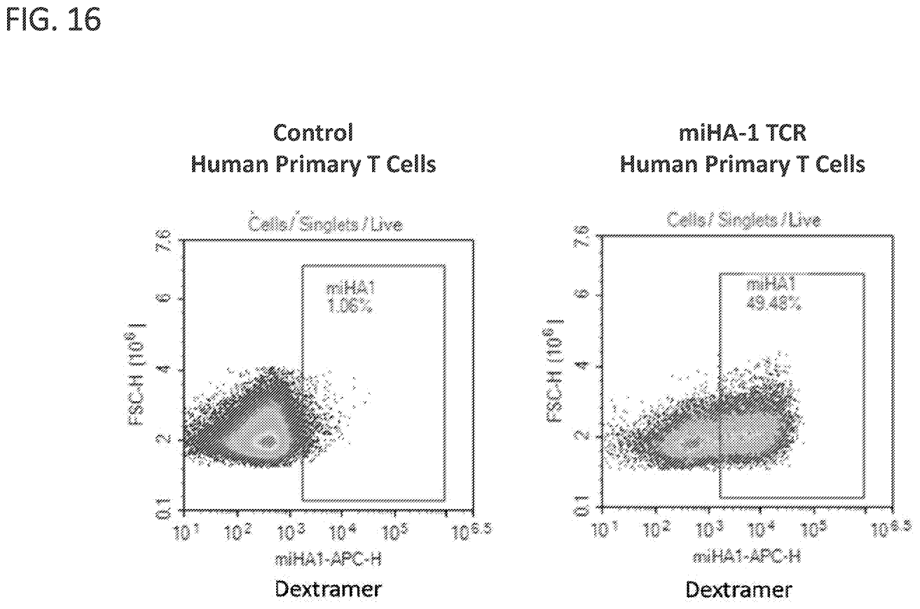

FIG. 16 shows the expression of an exemplary HA-1 TCR in human primary T cells, stained with an A*0201/HA-1 dextramer. As shown, nearly 50% of cells exhibited TCR expression following transduction.

### B. Target Cell Killing Specificity

The target cell killing specificity of an exemplary TCR was assessed. An LCL derived from a blood donor that was HLA typed was determined positive for the A*0201 MHC class I molecule and was used as an APC. The HA-1 genotype was R/R. Two populations of this LCL were stained either with high or low concentration of 5(6)-carboxyfluorescein diacetate N-succinimidyl ester (CFSE). CFSE labels target cells by binding of the dye to intracellular protein, and indicates T cell-mediated target cell killing. Apoptosis of labeled cells thereby results in the loss of their detection in the live cell gate in flow cytometry. The populations were loaded with the immunogenic “H” peptide, the non-immunogenic “R” peptide, or an unrelated (non-HA-1) peptide pp65 that binds to A*0201. Primary human T cells expressing an exemplary HA-1 TCR were co-cultured with the different target cells for 4 hours at an E:T ratio of 2.5:1. Cells were acquired and assessed by flow cytometry.

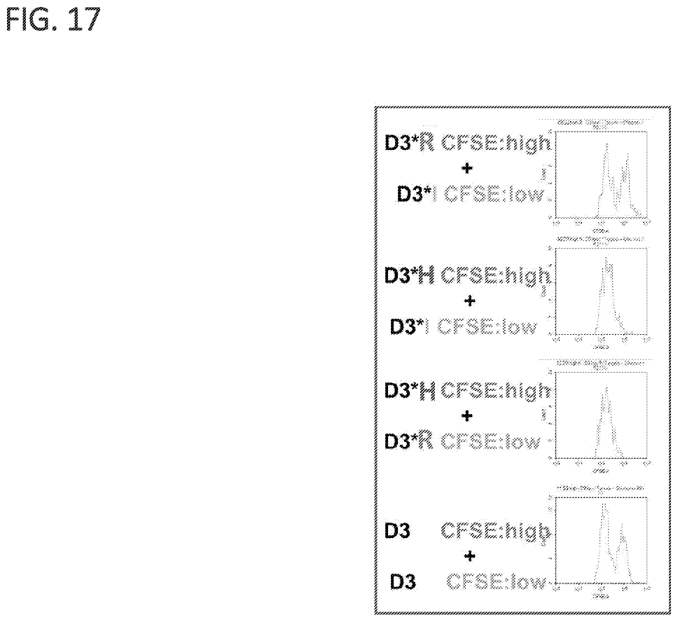

FIG. 17 shows flow cytometry plots showing populations of cells labeled with different CFSE levels, for peptide-loaded LCL populations following exposure to an exemplary HA-1 specific TCR. As shown in FIG. 17, when “H” peptide-loaded LCLs were present, only the non-“H” peptide-loaded populations were detected (second and third rows), showing specific elimination of “H” peptide-loaded LCLs. In comparison, when only “R” peptide-loaded LCLs or the unrelated pp65 peptide-loaded LCLs were present, both cell population labeled with high CFSE and low CFSE were observed (first and last rows). These results demonstrate specific killing of cells loaded with the immunogenic “H” peptide by the exemplary HA-1 specific TCRs, but not of cells loaded with the non-immunogenic “R” peptide or unrelated peptide pp65.

### C. Target Cell Killing Activity

The target cell killing activity of an exemplary TCR was further assessed at different effector:target (E:T) ratios.

LCLs were genotyped for the HA-1 haplotypes R/R (VLRDDLLEA) or H/H (VLHDDLLEA) (Astarte Biologics). In order to distinguish cell populations, LCLs presenting the “R” peptide were labeled with a low concentration of 5(6)-carboxyfluorescein diacetate N-succinimidyl ester (CFSE) (0.025 μM), while LCLs presenting the “H” peptide were labeled with a high concentration (0.5 μM) of CFSE. LCLs were stained with CFSE for 15 minutes at 37° C. HA-1 “H” or “R”-peptide-bearing LCLs were mixed together in a 1:1 ratio prior to incubation with increasing numbers of transduced primary T cells expressing an exemplary HA-1 specific TCR as identified and assessed in Examples 1˜4 above.

LCLs were co-cultured with increasing numbers of primary T cells for 16 hours (E:T ratios from 1:16 to 2:1). Cell mixtures were stained with LIVE/DEAD Fixable Violet Dead Cell Stain Kit (Thermo Fisher Scientific), anti-CD8 and anti-CD19, to assess the two distinct target cell populations by CFSE staining levels. Cells were acquired and analyzed by flow cytometry, assessing CD8− cells (to exclude effector cells) and the CFSE high versus CFSE low populations.

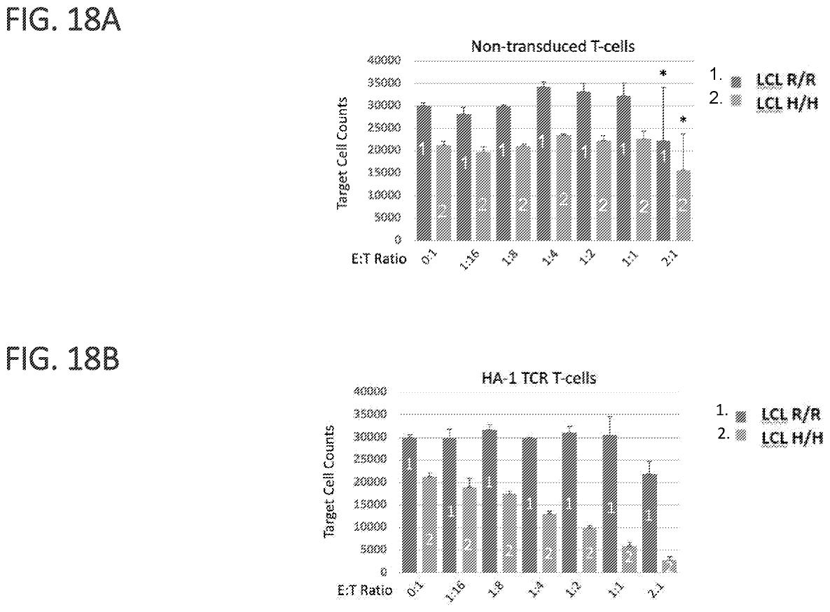

FIGS. 18A-18B show live cell counts of LCLs presenting either HA-1 “H” peptide or “R” peptide following incubation with non-transduced T cells and an exemplary anti-HA-1 TCR transduced T cells. As shown in FIGS. 18A-18B, loss of viable cells presenting HA-1 “H” peptide (high CFSE staining) was observed while numbers of viable cells presenting HA-1 “R” peptide (low CFSE staining) were retained, as the effector to target (E:T) ratio increased.

These results confirm that an exemplary anti-HA-1 TCR described in Examples 1˜4 above is capable of selective cell killing of target cells presenting the immunogenic HA-1 “H” peptide.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

TABLE 4
Sequences

| SEQ | | |
|---|---|---|
| ID | | |
| NO | SEQUENCE | Description |
| 1 | VSGNPY | TCR A, D, E |
| | | Vα CDR-1 |
| 2 | YITGDNLV | TCR A, D, E |
| | | Vα CDR-2 |
| 3 | AVRGGSYKYI | TCR A Vα |
| | | CDR-3 |
| 4 | AQSVAQPEDQVNVAEGNPLTVKCTYSVSGNPYLFWYVQYPNRGLQFLLKYITGDNLVKGSYGF | TCR A Vα |
| | EAEFNKSQTSFHLKKPSALVSDSALYFCAVRGGSYKYIFGTGTRLKVLAN | (aa) |
| 5 | MASAPISMLAMLFTLSGLRAQSVAQPEDQVNVAEGNPLTVKCTYSVSGNPYLFWYVQYPNRGL | TCR A Vα + |
| | QFLLKYITGDNLVKGSYGFEAEFNKSQTSFHLKKPSALVSDSALYFCAVRGGSYKYIFGTGTR | ss (aa) |
| | LKVLAN | |
| 6 | MASAPISMLAMLFTLSGLRAQSVAQPEDQVNVAEGNPLTVKCTYSVSGNPYLFWYVQYPNRGL | TCR A Vα + |
| | QFLLKYITGDNLVKGSYGFEAEFNKSQTSFHLKKPSALVSDSALYFCAVRGGSYKYIFGTGTR | Cα (aa) |
| | LKVLANIQNPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGFR | |
| | ILLLKVAGFNLLMTLRLWSS | |
| 7 | ATGGCCTCTGCACCCATCTCGATGCTTGCGATGCTCTTCACATTGAGTGGGCTGAGAGCTCAG | TCR A Vα |
| | TCAGTGGCTCAGCCGGAAGATCAGGTCAACGTTGCTGAAGGGAATCCTCTGACTGTGAAATGC | (nt) |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGGGGGTTCCTACAAATACATCTTTGGAACAGGCACCAGG | |
| | CTGAAGGTTTTAGCAAAT | |
| 8 | ATGGCCTCTGCACCCATCTCGATGCTTGCGATGCTCTTCACATTGAGTGGGCTGAGAGCTCAG | TCR A Vα + |
| | TCAGTGGCTCAGCCGGAAGATCAGGTCAACGTTGCTGAAGGGAATCCTCTGACTGTGAAATGC | Cα (nt) |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGGGGGTTCCTACAAATACATCTTTGGAACAGGCACCAGG | |
| | CTGAAGGTTTTAGCAAATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATGTGTCACAAAGT | |
| | AAGGATTCTGATGTGTATATCACAGACAAAACTGTGCTAGACATGAGGTCTATGGACTTCAAG | |
| | AGCAACAGTGCTGTGGCCTGGAGCAACAAATCTGACTTTGCATGTGCAAACGCCTTCAACAAC | |
| | AGCATTATTCCAGAAGACACCTTCTTCCCCAGCCCAGAAAGTTCCTGTGATGTCAAGCTGGTC | |
| | GAGAAAAGCTTTGAAACAGATACGAACCTAAACTTTCAAAACCTGTCAGTGATTGGGTTCCGA | |
| | ATCCTCCTCCTGAAAGTGGCCGGGTTTAATCTGCTCATGACGCTGCGGCTGTGGTCCAGC | |
| 9 | SEHNR | TCR A-T Vβ |
| | | CDR-1 |
| 10 | FQNEAQ | TCR A-T Vβ |
| | | CDR-2 |
| 11 | ASTPGTVYNEQF | TCR A Vβ |
| | | CDR-3 |
| 12 | DTGVSQDPRHKITKRGQNVTFRCDPISEHNRLYWYRQTLGQGPEFLTYFQNEAQLEKSRLLSD | TCR A Vβ |
| | RFSAERPKGSFSTLEIQRTEQGDSAMYLCASTPGTVYNEQFFGPGTRLTVLE | (aa) |
| 13 | MGTSLLCWMALCLLGADHADTGVSQDPRHKITKRGQNVTFRCDPISEHNRLYWYRQTLGQGPE | TCR A Vβ + |
| | FLTYFQNEAQLEKSRLLSDRFSAERPKGSFSTLEIQRTEQGDSAMYLCASTPGTVYNEQFFGP | ss (aa) |
| | GTRLTVLE | |
| 14 | MGTSLLCWMALCLLGADHADTGVSQDPRHKITKRGQNVTFRCDPISEHNRLYWYRQTLGQGPE | TCR A Vβ + |
| | FLTYFQNEAQLEKSRLLSDRFSAERPKGSFSTLEIQRTEQGDSAMYLCASTPGTVYNEQFFGP | Cβ (aa) |
| | GTRLTVLEDLKNVFPPEVAVFEPSEAEISHTQKATLVCLATGFYPDHVELSWWVNGKEVHSGV | |
| | STDPQPLKEQPALNDSRYCLSSRLRVSATFWQNPRNHFRCQVQFYGLSENDEWTQDRAKPVTQ | |
| | IVSAEAWGRADCGFTSESYQQGVLSATILYEILLGKATLYAVLVSALVLMAMVKRKDSRG | |
| 15 | ATGGGTACCAGCCTCCTCTGCTGGATGGCCCTGTGTCTCCTGGGGGCAGATCACGCAGATACT | TCR A Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCACCCCCGGGACGGTCTACAATGAGCAGTTCTTCGGGCCA | |
| | GGGACACGGCTCACCGTGCTAGAG | |
| 16 | ATGGGTACCAGCCTCCTCTGCTGGATGGCCCTGTGTCTCCTGGGGGCAGATCACGCAGATACT | TCR A Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCACCCCCGGGACGGTCTACAATGAGCAGTTCTTCGGGCCA | |
| | GGGACACGGCTCACCGTGCTAGAGGACCTGAAAAACGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 17 | ATGGCCTCTGCACCCATCTCGATGCTTGCGATGCTCTTCACATTGAGTGGGCTGAGAGCTCAG | TCR A full |
| | TCAGTGGCTCAGCCGGAAGATCAGGTCAACGTTGCTGAAGGGAATCCTCTGACTGTGAAATGC | construct nt |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGGGGGTTCCTACAAATACATCTTTGGAACAGGCACCAGG | |
| | CTGAAGGTTTTAGCAAATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATGTGTCACAAAGT | |
| | AAGGATTCTGATGTGTATATCACAGACAAAACTGTGCTAGACATGAGGTCTATGGACTTCAAG | |
| | AGCAACAGTGCTGTGGCCTGGAGCAACAAATCTGACTTTGCATGTGCAAACGCCTTCAACAAC | |
| | AGCATTATTCCAGAAGACACCTTCTTCCCCAGCCCAGAAAGTTCCTGTGATGTCAAGCTGGTC | |
| | GAGAAAAGCTTTGAAACAGATACGAACCTAAACTTTCAAAACCTGTCAGTGATTGGGTTCCGA | |
| | ATCCTCCTCCTGAAAGTGGCCGGGTTTAATCTGCTCATGACGCTGCGGCTGTGGTCCAGCGGC | |
| | TCCGGAGCCACGAACTTCTCTCTGTTAAAGCAAGCAGGAGACGTGGAAGAAAACCCCGGTCCC | |
| | ATGGGTACCAGCCTCCTCTGCTGGATGGCCCTGTGTCTCCTGGGGGCAGATCACGCAGATACT | |
| | GGAGTCTCCCAGGACCCCAGACACAAGATCACAAAGAGGGGACAGAATGTAACTTTCAGGTGT | |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCACCCCCGGGACGGTCTACAATGAGCAGTTCTTCGGGCCA | |
| | GGGACACGGCTCACCGTGCTAGAGGACCTGAAAAACGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 18 | MASAPISMLAMLFTLSGLRAQSVAQPEDQVNVAEGNPLTVKCTYSVSGNPYLFWYVQYPNRGL | TCR A full |
| | QFLLKYITGDNLVKGSYGFEAEFNKSQTSFHLKKPSALVSDSALYFCAVRGGSYKYIFGTGTR | construct aa |
| | LKVLANIQNPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGFR | |
| | ILLLKVAGFNLLMTLRLWSSGSGATNFSLLKQAGDVEENPGPMGTSLLCWMALCLLGADHADT | |
| | GVSQDPRHKITKRGQNVTFRCDPISEHNRLYWYRQTLGQGPEFLTYFQNEAQLEKSRLLSDRF | |
| | SAERPKGSFSTLEIQRTEQGDSAMYLCASTPGTVYNEQFFGPGTRLTVLEDLKNVFPPEVAVF | |
| | EPSEAEISHTQKATLVCLATGFYPDHVELSWWVNGKEVHSGVSTDPQPLKEQPALNDSRYCLS | |
| | SRLRVSATFWQNPRNHFRCQVQFYGLSENDEWTQDRAKPVTQIVSAEAWGRADCGFTSESYQQ | |
| | GVLSATILYEILLGKATLYAVLVSALVLMAMVKRKDSRG | |
| 19 | TSINN | TCR B, G, R, |
| | | T Vα CDR-1 |
| 20 | IRSNERE | TCR B, G, R, |
| | | T Vα CDR-2 |
| 21 | ATAPGSGTYKYI | TCR B Vα |
| | | CDR-3 |
| 22 | SQQGEEDPQALSIQEGENATMNCSYKTSINNLQWYRQNSGRGLVHLILIRSNEREKHSGRLRV | TCR B Vα |
| | TLDTSKKSSSLLITASRAADTASYFCATAPGSGTYKYIFGTGTRLKVLAN | (aa) |
| 23 | METLLGVSLVILWLQLARVNSQQGEEDPQALSIQEGENATMNCSYKTSINNLQWYRQNSGRGL | TCR B Vα + |
| | VHLILIRSNEREKHSGRLRVTLDTSKKSSSLLITASRAADTASYFCATAPGSGTYKYIFGTGT | ss (aa) |
| | RLKVLAN | |
| 24 | METLLGVSLVILWLQLARVNSQQGEEDPQALSIQEGENATMNCSYKTSINNLQWYRQNSGRGL | TCR B Vα + |
| | VHLILIRSNEREKHSGRLRVTLDTSKKSSSLLITASRAADTASYFCATAPGSGTYKYIFGTGT | Cα (aa) |
| | RLKVLANIQNPDPAVYQLRDSKSSDKSVCLFTDFDSQTNVSQSKDSDVYITDKTVLDMRSMDF | |
| | KSNSAVAWSNKSDFACANAFNNSIIPEDTFFPSPESSCDVKLVEKSFETDTNLNFQNLSVIGF | |
| | RILLLKVAGFNLLMTLRLWSS | |
| 25 | ATGGAAACTCTCCTGGGAGTGTCTTTGGTGATTCTATGGCTTCAACTGGCTAGGGTGAACAGT | TCR B Vα |
| | CAACAGGGAGAAGAGGATCCTCAGGCCTTGAGCATCCAGGAGGGTGAAAATGCCACCATGAAC | (nt) |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACGGCCCCTGGCTCAGGAACCTACAAATACATCTTTGGAACAGGCACC | |
| | AGGCTGAAGGTTTTAGCAAAT | |
| 26 | ATGGAAACTCTCCTGGGAGTGTCTTTGGTGATTCTATGGCTTCAACTGGCTAGGGTGAACAGT | TCR B Vα + |
| | CAACAGGGAGAAGAGGATCCTCAGGCCTTGAGCATCCAGGAGGGTGAAAATGCCACCATGAAC | Cα (nt) |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACGGCCCCTGGCTCAGGAACCTACAAATACATCTTTGGAACAGGCACC | |
| | AGGCTGAAGGTTTTAGCAAATATCCAGAACCCTGACCCTGCCGTGTACCAGCTGAGAGACTCT | |
| | AAATCCAGTGACAAGTCTGTCTGCCTATTCACCGATTTTGATTCTCAAACAAATGTGTCACAA | |
| | AGTAAGGATTCTGATGTGTATATCACAGACAAAACTGTGCTAGACATGAGGTCTATGGACTTC | |
| | AAGAGCAACAGTGCTGTGGCCTGGAGCAACAAATCTGACTTTGCATGTGCAAACGCCTTCAAC | |
| | AACAGCATTATTCCAGAAGACACCTTCTTCCCCAGCCCAGAAAGTTCCTGTGATGTCAAGCTG | |
| | GTCGAGAAAAGCTTTGAAACAGATACGAACCTAAACTTTCAAAACCTGTCAGTGATTGGGTTC | |
| | CGAATCCTCCTCCTGAAAGTGGCCGGGTTTAATCTGCTCATGACGCTGCGGCTGTGGTCCAGC | |
| 27 | AAPPDTYNSPLH | TCR B Vβ |
| | | CDR-3 |
| 28 | DTGVSQDPRHKITKRGQNVTFRCDPISEHNRLYWYRQTLGQGPEFLTYFQNEAQLEKSRLLSD | TCR B Vβ |
| | RFSAERPKGSFSTLEIQRTEQGDSAMYLCAAPPDTYNSPLHFGNGTRLTVTE | (aa) |
| 29 | MGTSLLCWMALCLLGADHADTGVSQDPRHKITKRGQNVTFRCDPISEHNRLYWYRQTLGQGPE | TCR B Vβ + |
| | FLTYFQNEAQLEKSRLLSDRFSAERPKGSFSTLEIQRTEQGDSAMYLCAAPPDTYNSPLHFGN | ss (aa) |
| | GTRLTVTE | |
| 30 | MGTSLLCWMALCLLGADHADTGVSQDPRHKITKRGQNVTFRCDPISEHNRLYWYRQTLGQGPE | TCR B Vβ + |
| | FLTYFQNEAQLEKSRLLSDRFSAERPKGSFSTLEIQRTEQGDSAMYLCAAPPDTYNSPLHFGN | Cβ (aa) |
| | GTRLTVTEDLNKVEPPEVAVFEPSEAEISHTQKATLVCLATGFFPDHVELSWWVNGKEVHSGV | |
| | STDPQPLKEQPALNDSRYCLSSRLRVSATFWQNPRNHERCQVQFYGLSENDEWTQDRAKPVTQ | |
| | IVSAEAWGRADCGFTSVSYQQGVLSATILYEILLGKATLYAVLVSALVLMAMVKRKDF | |
| 31 | ATGGGTACCAGCCTCCTCTGCTGGATGGCCCTGTGTCTCCTGGGGGCAGATCACGCAGATACT | TCR B Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCGCCCCCCCGGACACCTATAATTCACCCCTCCACTTTGGGAAT | |
| | GGGACCAGGCTCACTGTGACAGAG | |
| 32 | ATGGGTACCAGCCTCCTCTGCTGGATGGCCCTGTGTCTCCTGGGGGCAGATCACGCAGATACT | TCR B Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCGCCCCCCCGGACACCTATAATTCACCCCTCCACTTTGGGAAT | |
| | GGGACCAGGCTCACTGTGACAGAGGACCTGAACAAGGTGTTCCCACCCGAGGTCGCTGTGTTT | |
| | GAGCCATCAGAAGCAGAGATCTCCCACACCCAAAAGGCCACACTGGTGTGCCTGGCCACAGGC | |
| | TTCTTCCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCCGTCACCCAG | |
| | ATCGTCAGCGCCGAGGCCTGGGGTAGAGCAGACTGTGGCTTTACCTCGGTGTCCTACCAGCAA | |
| | GGGGTCCTGTCTGCCACCATCCTCTATGAGATCCTGCTAGGGAAGGCCACCCTGTATGCTGTG | |
| | CTGGTCAGCGCCCTTGTGTTGATGGCCATGGTCAAGAGAAAGGATTTCTGA | |
| 33 | ATGGAAACTCTCCTGGGAGTGTCTTTGGTGATTCTATGGCTTCAACTGGCTAGGGTGAACAGT | TCR B full |
| | CAACAGGGAGAAGAGGATCCTCAGGCCTTGAGCATCCAGGAGGGTGAAAATGCCACCATGAAC | construct nt |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACGGCCCCTGGCTCAGGAACCTACAAATACATCTTTGGAACAGGCACC | |
| | AGGCTGAAGGTTTTAGCAAATATCCAGAACCCTGACCCTGCCGTGTACCAGCTGAGAGACTCT | |
| | AAATCCAGTGACAAGTCTGTCTGCCTATTCACCGATTTTGATTCTCAAACAAATGTGTCACAA | |
| | AGTAAGGATTCTGATGTGTATATCACAGACAAAACTGTGCTAGACATGAGGTCTATGGACTTC | |
| | AAGAGCAACAGTGCTGTGGCCTGGAGCAACAAATCTGACTTTGCATGTGCAAACGCCTTCAAC | |
| | AACAGCATTATTCCAGAAGACACCTTCTTCCCCAGCCCAGAAAGTTCCTGTGATGTCAAGCTG | |
| | GTCGAGAAAAGCTTTGAAACAGATACGAACCTAAACTTTCAAAACCTGTCAGTGATTGGGTTC | |
| | CGAATCCTCCTCCTGAAAGTGGCCGGGTTTAATCTGCTCATGACGCTGCGGCTGTGGTCCAGC | |
| | GGCTCCGGAGCCACGAACTTCTCTCTGTTAAAGCAAGCAGGAGACGTGGAAGAAAACCCCGGT | |
| | CCCATGGGTACCAGCCTCCTCTGCTGGATGGCCCTGTGTCTCCTGGGGGCAGATCACGCAGAT | |
| | ACTGGAGTCTCCCAGGACCCCAGACACAAGATCACAAAGAGGGGACAGAATGTAACTTTCAGG | |
| | TGTGATCCAATTTCTGAACACAACCGCCTTTATTGGTACCGACAGACCCTGGGGCAGGGCCCA | |
| | GAGTTTCTGACTTACTTCCAGAATGAAGCTCAACTAGAAAAATCAAGGCTGCTCAGTGATCGG | |
| | TTCTCTGCAGAGAGGCCTAAGGGATCTTTCTCCACCTTGGAGATCCAGCGCACAGAGCAGGGG | |
| | GACTCGGCCATGTATCTCTGTGCCGCCCCCCCGGACACCTATAATTCACCCCTCCACTTTGGG | |
| | AATGGGACCAGGCTCACTGTGACAGAGGACCTGAACAAGGTGTTCCCACCCGAGGTCGCTGTG | |
| | TTTGAGCCATCAGAAGCAGAGATCTCCCACACCCAAAAGGCCACACTGGTGTGCCTGGCCACA | |
| | GGCTTCTTCCCCGACCACGTGGAGCTGAGCTGGTGGGTGAATGGGAAGGAGGTGCACAGTGGG | |
| | GTCAGCACAGACCCGCAGCCCCTCAAGGAGCAGCCCGCCCTCAATGACTCCAGATACTGCCTG | |
| | AGCAGCCGCCTGAGGGTCTCGGCCACCTTCTGGCAGAACCCCCGCAACCACTTCCGCTGTCAA | |
| | GTCCAGTTCTACGGGCTCTCGGAGAATGACGAGTGGACCCAGGATAGGGCCAAACCCGTCACC | |
| | CAGATCGTCAGCGCCGAGGCCTGGGGTAGAGCAGACTGTGGCTTTACCTCGGTGTCCTACCAG | |
| | CAAGGGGTCCTGTCTGCCACCATCCTCTATGAGATCCTGCTAGGGAAGGCCACCCTGTATGCT | |
| | GTGCTGGTCAGCGCCCTTGTGTTGATGGCCATGGTCAAGAGAAAGGATTTCTGA | |
| 34 | METLLGVSLVILWLQLARVNSQQGEEDPQALSIQEGENATMNCSYKTSINNLQWYRQNSGRGL | TCR B full |
| | VHLILIRSNEREKHSGRLRVTLDTSKKSSSLLITASRAADTASYFCATAPGSGTYKYIFGTGT | construct aa |
| | RLKVLANIQNPDPAVYQLRDSKSSDKSVCLFTDFDSQTNVSQSKDSDVYITDKTVLDMRSMDF | |
| | KSNSAVAWSNKSDFACANAFNNSIIPEDTFFPSPESSCDVKLVEKSFETDTNLNFQNLSVIGF | |
| | RILLLKVAGFNLLMTLRLWSSGSGATNFSLLKQAGDVEENPGPMGTSLLCWMALCLLGADHAD | |
| | TGVSQDPRHKITKRGQNVTFRCDPISEHNRLYWYRQTLGQGPEFLTYFQNEAQLEKSRLLSDR | |
| | FSAERPKGSFSTLEIQRTEQGDSAMYLCAAPPDTYNSPLHFGNGTRLTVTEDLNKVEPPEVAV | |
| | FEPSEAEISHTQKATLVCLATGFFPDHVELSWWVNGKEVHSGVSTDPQPLKEQPALNDSRYCL | |
| | SSRLRVSATFWQNPRNHFRCQVQFYGLSENDEWTQDRAKPVTQIVSAEAWGRADCGFTSVSYQ | |
| | QGVLSATILYEILLGKATLYAVLVSALVLMAMVKRKDF* | |
| 35 | DSAIYN | TCR C, I, L, |
| | | M Vα CDR-1 |
| 36 | IQSSQRE | TCR C, I, L, |
| | | M Vα CDR-2 |
| 37 | AVRPRTSGTYKYI | TCR C Vα |
| | | CDR-3 |
| 38 | KQEVTQIPAALSVPEGENLVLNCSFTDSAIYNLQWFRQDPGKGLTSLLLIQSSQREQTSGRLN | TCR C Vα |
| | ASLDKSSGRSTLYIAASQPGDSATYLCAVRPRTSGTYKYIFGTGTRLKVLAN | (aa) |
| 39 | METLLGLLILWLQLQWVSSKQEVTQIPAALSVPEGENLVLNCSFTDSAIYNLQWFRQDPGKGL | TCR C Vα + |
| | TSLLLIQSSQREQTSGRLNASLDKSSGRSTLYIAASQPGDSATYLCAVRPRTSGTYKYIFGTG | ss (aa) |
| | TRLKVLAN | |
| 40 | METLLGLLILWLQLQWVSSKQEVTQIPAALSVPEGENLVLNCSFTDSAIYNLQWFRQDPGKGL | TCR C Vα + |
| | TSLLLIQSSQREQTSGRLNASLDKSSGRSTLYIAASQPGDSATYLCAVRPRTSGTYKYIFGTG | Cα (aa) |
| | TRLKVLANIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSS | |
| 41 | ATGGAGACCCTCTTGGGCCTGCTTATCCTTTGGCTGCAGCTGCAATGGGTGAGCAGCAAACAG | TCR C Vα |
| | GAGGTGACGCAGATTCCTGCAGCTCTGAGTGTCCCAGAAGGAGAAAACTTGGTTCTCAACTGC | (nt) |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTGAGGCCTCGGACCTCAGGAACCTACAAATACATCTTTGGAACAGGC | |
| | ACCAGGCTGAAGGTTTTAGCAAAT | |
| 42 | ATGGAGACCCTCTTGGGCCTGCTTATCCTTTGGCTGCAGCTGCAATGGGTGAGCAGCAAACAG | TCR C Vα + |
| | GAGGTGACGCAGATTCCTGCAGCTCTGAGTGTCCCAGAAGGAGAAAACTTGGTTCTCAACTGC | Cα (nt) |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTGAGGCCTCGGACCTCAGGAACCTACAAATACATCTTTGGAACAGGC | |
| | ACCAGGCTGAAGGTTTTAGCAAATATCCAGAACCCTGACCCTGCCGTGTACCAGCTGAGAGAC | |
| | TCTAAATCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGTCTATGGAC | |
| | TTCAAGAGCAACAGTGCTGTGGCCTGGAGCAACAAATCTGACTTTGCATGTGCAAACGCCTTC | |
| | AACAACAGCATTATTCCAGAAGACACCTTCTTCCCCAGCCCAGAAAGTTCCTGTGATGTCAAG | |
| | CTGGTCGAGAAAAGCTTTGAAACAGATACGAACCTAAACTTTCAAAACCTGTCAGTGATTGGG | |
| | TTCCGAATCCTCCTCCTGAAAGTGGCCGGGTTTAATCTGCTCATGACGCTGCGGCTGTGGTCC | |
| | AGC | |
| 43 | ASTELSGNTIY | TCR C Vβ |
| | | CDR-3 |
| 44 | DTGVSQDPRHKITKRGQNVTFRCDPISEHNRLYWYRQTLGQGPEFLTYFQNEAQLEKSRLLSD | TCR C Vβ |
| | RFSAERPKGSFSTLEIQRTEQGDSAMYLCASTELSGNTIYFGEGSWLTVVE | (aa) |
| 45 | MGTSLLCWMALCLLGADHADTGVSQDPRHKITKRGQNVTFRCDPISEHNRLYWYRQTLGQGPE | TCR C Vβ + |
| | FLTYFQNEAQLEKSRLLSDRFSAERPKGSFSTLEIQRTEQGDSAMYLCASTELSGNTIYFGEG | ss (aa) |
| | SWLTVVE | |
| 46 | MGTSLLCWMALCLLGADHADTGVSQDPRHKITKRGQNVTFRCDPISEHNRLYWYRQTLGQGPE | TCR C Vβ + |
| | FLTYFQNEAQLEKSRLLSDRFSAERPKGSFSTLEIQRTEQGDSAMYLCASTELSGNTIYFGEG | Cβ (aa) |
| | SWLTVVEDLNKVFPPEVAVFEPSEAEISHTQKATLVCLATGFFPDHVELSWWVNGKEVHSGVS | |
| | TDPQPLKEQPALNDSRYCLSSRLRVSATFWQNPRNHFRCQVQFYGLSENDEWTQDRAKPVTQI | |
| | VSAEAWGRADCGFTSVSYQQGVLSATILYEILLGKATLYAVLVSALVLMAMVKRKDF | |
| 47 | ATGGGTACCAGCCTCCTCTGCTGGATGGCCCTGTGTCTCCTGGGGGCAGATCACGCAGATACT | TCR C Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCACAGAACTCTCTGGAAACACCATATATTTTGGAGAGGGA | |
| | AGTTGGCTCACTGTTGTAGAG | |
| 48 | ATGGGTACCAGCCTCCTCTGCTGGATGGCCCTGTGTCTCCTGGGGGCAGATCACGCAGATACT | TCR C Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCACAGAACTCTCTGGAAACACCATATATTTTGGAGAGGGA | |
| | AGTTGGCTCACTGTTGTAGAGGACCTGAACAAGGTGTTCCCACCCGAGGTCGCTGTGTTTGAG | |
| | CCATCAGAAGCAGAGATCTCCCACACCCAAAAGGCCACACTGGTGTGCCTGGCCACAGGCTTC | |
| | TTCCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCCGTCACCCAGATC | |
| | GTCAGCGCCGAGGCCTGGGGTAGAGCAGACTGTGGCTTTACCTCGGTGTCCTACCAGCAAGGG | |
| | GTCCTGTCTGCCACCATCCTCTATGAGATCCTGCTAGGGAAGGCCACCCTGTATGCTGTGCTG | |
| | GTCAGCGCCCTTGTGTTGATGGCCATGGTCAAGAGAAAGGATTTCTGA | |
| 49 | ATGGAGACCCTCTTGGGCCTGCTTATCCTTTGGCTGCAGCTGCAATGGGTGAGCAGCAAACAG | TCR C full |
| | GAGGTGACGCAGATTCCTGCAGCTCTGAGTGTCCCAGAAGGAGAAAACTTGGTTCTCAACTGC | construct nt |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTGAGGCCTCGGACCTCAGGAACCTACAAATACATCTTTGGAACAGGC | |
| | ACCAGGCTGAAGGTTTTAGCAAATATCCAGAACCCTGACCCTGCCGTGTACCAGCTGAGAGAC | |
| | TCTAAATCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGTCTATGGAC | |
| | TTCAAGAGCAACAGTGCTGTGGCCTGGAGCAACAAATCTGACTTTGCATGTGCAAACGCCTTC | |
| | AACAACAGCATTATTCCAGAAGACACCTTCTTCCCCAGCCCAGAAAGTTCCTGTGATGTCAAG | |
| | CTGGTCGAGAAAAGCTTTGAAACAGATACGAACCTAAACTTTCAAAACCTGTCAGTGATTGGG | |
| | TTCCGAATCCTCCTCCTGAAAGTGGCCGGGTTTAATCTGCTCATGACGCTGCGGCTGTGGTCC | |
| | AGCGGCTCCGGAGCCACGAACTTCTCTCTGTTAAAGCAAGCAGGAGACGTGGAAGAAAACCCC | |
| | GGTCCCATGGGTACCAGCCTCCTCTGCTGGATGGCCCTGTGTCTCCTGGGGGCAGATCACGCA | |
| | GATACTGGAGTCTCCCAGGACCCCAGACACAAGATCACAAAGAGGGGACAGAATGTAACTTTC | |
| | AGGTGTGATCCAATTTCTGAACACAACCGCCTTTATTGGTACCGACAGACCCTGGGGCAGGGC | |
| | CCAGAGTTTCTGACTTACTTCCAGAATGAAGCTCAACTAGAAAAATCAAGGCTGCTCAGTGAT | |
| | CGGTTCTCTGCAGAGAGGCCTAAGGGATCTTTCTCCACCTTGGAGATCCAGCGCACAGAGCAG | |
| | GGGGACTCGGCCATGTATCTCTGTGCCAGCACAGAACTCTCTGGAAACACCATATATTTTGGA | |
| | GAGGGAAGTTGGCTCACTGTTGTAGAGGACCTGAACAAGGTGTTCCCACCCGAGGTCGCTGTG | |
| | TTTGAGCCATCAGAAGCAGAGATCTCCCACACCCAAAAGGCCACACTGGTGTGCCTGGCCACA | |
| | GGCTTCTTCCCCGACCACGTGGAGCTGAGCTGGTGGGTGAATGGGAAGGAGGTGCACAGTGGG | |
| | GTCAGCACAGACCCGCAGCCCCTCAAGGAGCAGCCCGCCCTCAATGACTCCAGATACTGCCTG | |
| | AGCAGCCGCCTGAGGGTCTCGGCCACCTTCTGGCAGAACCCCCGCAACCACTTCCGCTGTCAA | |
| | GTCCAGTTCTACGGGCTCTCGGAGAATGACGAGTGGACCCAGGATAGGGCCAAACCCGTCACC | |
| | CAGATCGTCAGCGCCGAGGCCTGGGGTAGAGCAGACTGTGGCTTTACCTCGGTGTCCTACCAG | |
| | CAAGGGGTCCTGTCTGCCACCATCCTCTATGAGATCCTGCTAGGGAAGGCCACCCTGTATGCT | |
| | GTGCTGGTCAGCGCCCTTGTGTTGATGGCCATGGTCAAGAGAAAGGATTTCTGA | |
| 50 | METLLGLLILWLQLQWVSSKQEVTQIPAALSVPEGENLVLNCSFTDSAIYNLQWFRQDPGKGL | TCR C full |
| | TSLLLIQSSQREQTSGRLNASLDKSSGRSTLYIAASQPGDSATYLCAVRPRTSGTYKYIFGTG | construct aa |
| | TRLKVLANIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSSGSGATNFSLLKQAGDVEENPGPMGTSLLCWMALCLLGADHA | |
| | DTGVSQDPRHKITKRGQNVTFRCDPISEHNRLYWYRQTLGQGPEFLTYFQNEAQLEKSRLLSD | |
| | RFSAERPKGSFSTLEIQRTEQGDSAMYLCASTELSGNTIYFGEGSWLTVVEDLNKVFPPEVAV | |
| | FEPSEAEISHTQKATLVCLATGFFPDHVELSWWVNGKEVHSGVSTDPQPLKEQPALNDSRYCL | |
| | SSRLRVSATFWQNPRNHFRCQVQFYGLSENDEWTQDRAKPVTQIVSAEAWGRADCGFTSVSYQ | |
| | QGVLSATILYEILLGKATLYAVLVSALVLMAMVKRKDF | |
| 51 | AVRAPTSGTYKYI | TCR D Vα |
| | | CDR-3 |
| 52 | AQSVAQPEDQVNVAEGNPLTVKCTYSVSGNPYLFWYVQYPNRGLQFLLKYITGDNLVKGSYGF | TCR D Vα |
| | EAEFNKSQTSFHLKKPSALVSDSALYFCAVRAPTSGTYKYIFGTGTRLKVLAN | (aa) |
| 53 | MASAPISMLAMLFTLSGLRAQSVAQPEDQVNVAEGNPLTVKCTYSVSGNPYLFWYVQYPNRGL | TCR D Vα + |
| | QFLLKYITGDNLVKGSYGFEAEFNKSQTSFHLKKPSALVSDSALYFCAVRAPTSGTYKYIFGT | ss (aa) |
| | GTRLKVLAN | |
| 54 | MASAPISMLAMLFTLSGLRAQSVAQPEDQVNVAEGNPLTVKCTYSVSGNPYLFWYVQYPNRGL | TCR D Vα + |
| | QFLLKYITGDNLVKGSYGFEAEFNKSQTSFHLKKPSALVSDSALYFCAVRAPTSGTYKYIFGT | Cα (aa) |
| | GTRLKVLANIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGFNLLMTLRLWSS | |
| 55 | ATGGCCTCTGCACCCATCTCGATGCTTGCGATGCTCTTCACATTGAGTGGGCTGAGAGCTCAG | TCR D Vα |
| | TCAGTGGCTCAGCCGGAAGATCAGGTCAACGTTGCTGAAGGGAATCCTCTGACTGTGAAATGC | (nt) |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGCTCCTACCTCAGGAACCTACAAATACATCTTTGGAACA | |
| | GGCACCAGGCTGAAGGTTTTAGCAAAT | |
| 56 | ATGGCCTCTGCACCCATCTCGATGCTTGCGATGCTCTTCACATTGAGTGGGCTGAGAGCTCAG | TCR D Vα + |
| | TCAGTGGCTCAGCCGGAAGATCAGGTCAACGTTGCTGAAGGGAATCCTCTGACTGTGAAATGC | Cα (nt) |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGCTCCTACCTCAGGAACCTACAAATACATCTTTGGAACA | |
| | GGCACCAGGCTGAAGGTTTTAGCAAATATCCAGAACCCTGACCCTGCCGTGTACCAGCTGAGA | |
| | GACTCTAAATCCAGTGACAAGTCTGTCTGCCTATTCACCGATTTTGATTCTCAAACAAATGTG | |
| | TCACAAAGTAAGGATTCTGATGTGTATATCACAGACAAAACTGTGCTAGACATGAGGTCTATG | |
| | GACTTCAAGAGCAACAGTGCTGTGGCCTGGAGCAACAAATCTGACTTTGCATGTGCAAACGCC | |
| | TTCAACAACAGCATTATTCCAGAAGACACCTTCTTCCCCAGCCCAGAAAGTTCCTGTGATGTC | |
| | AAGCTGGTCGAGAAAAGCTTTGAAACAGATACGAACCTAAACTTTCAAAACCTGTCAGTGATT | |
| | GGGTTCCGAATCCTCCTCCTGAAAGTGGCCGGGTTTAATCTGCTCATGACGCTGCGGCTGTGG | |
| | TCCAGC | |
| 57 | ASSLVSGNEQF | TCR D, E Vβ |
| | | CDR-3 |
| 58 | DTGVSQDPRHKITKRGQNVTFRCDPISEHNRLYWYRQTLGQGPEFLTYFQNEAQLEKSRLLSD | TCR D, E Vβ |
| | RFSAERPKGSFSTLEIQRTEQGDSAMYLCASSLVSGNEQFFGPGTRLTVLE | (aa) |
| 59 | MGTSLLCWMALCLLGADHADTGVSQDPRHKITKRGQNVTFRCDPISEHNRLYWYRQTLGQGPE | TCR D Vβ + |
| | FLTYFQNEAQLEKSRLLSDRFSAERPKGSFSTLEIQRTEQGDSAMYLCASSLVSGNEQFFGPG | ss (aa) |
| | TRLTVLE | |
| 60 | MGTSLLCWMALCLLGADHADTGVSQDPRHKITKRGQNVTFRCDPISEHNRLYWYRQTLGQGPE | TCR D Vβ + |
| | FLTYFQNEAQLEKSRLLSDRFSAERPKGSFSTLEIQRTEQGDSAMYLCASSLVSGNEQFFGPG | Cβ (aa) |
| | TRLTVLEDLKNVFPPEVAVFEPSEAEISHTQKATLVCLATGFYPDHVELSWWVNGKEVHSGVS | |
| | TDPQPLKEQPALNDSRYCLSSRLRVSATFWQNPRNHERCQVQFYGLSENDEWTQDRAKPVTQI | |
| | VSAEAWGRADCGFTSESYQQGVLSATILYEILLGKATLYAVLVSALVLMAMVKRKDSRG | |
| 61 | ATGGGTACCAGCCTCCTCTGCTGGATGGCCCTGTGTCTCCTGGGGGCAGATCACGCAGATACT | TCR D Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGTCTCGGGCAATGAGCAGTTCTTCGGGCCAGGG | |
| | ACACGGCTCACCGTGCTAGAG | |
| 62 | ATGGGTACCAGCCTCCTCTGCTGGATGGCCCTGTGTCTCCTGGGGGCAGATCACGCAGATACT | TCR D Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGTCTCGGGCAATGAGCAGTTCTTCGGGCCAGGG | |
| | ACACGGCTCACCGTGCTAGAGGACCTGAAAAACGTGTTCCCACCCGAGGTCGCTGTGTTTGAG | |
| | CCATCAGAAGCAGAGATCTCCCACACCCAAAAGGCCACACTGGTATGCCTGGCCACAGGCTTC | |
| | TACCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCTGTCACCCAGATC | |
| | GTCAGCGCCGAGGCCTGGGGTAGAGCAGACTGTGGCTTCACCTCCGAGTCTTACCAGCAAGGG | |
| | GTCCTGTCTGCCACCATCCTCTATGAGATCTTGCTAGGGAAGGCCACCTTGTATGCCGTGCTG | |
| | GTCAGTGCCCTCGTGCTGATGGCCATGGTCAAGAGAAAGGATTCCAGAGGCTAG | |
| 63 | ATGGCCTCTGCACCCATCTCGATGCTTGCGATGCTCTTCACATTGAGTGGGCTGAGAGCTCAG | TCR D full |
| | TCAGTGGCTCAGCCGGAAGATCAGGTCAACGTTGCTGAAGGGAATCCTCTGACTGTGAAATGC | construct nt |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGCTCCTACCTCAGGAACCTACAAATACATCTTTGGAACA | |
| | GGCACCAGGCTGAAGGTTTTAGCAAATATCCAGAACCCTGACCCTGCCGTGTACCAGCTGAGA | |
| | GACTCTAAATCCAGTGACAAGTCTGTCTGCCTATTCACCGATTTTGATTCTCAAACAAATGTG | |
| | TCACAAAGTAAGGATTCTGATGTGTATATCACAGACAAAACTGTGCTAGACATGAGGTCTATG | |
| | GACTTCAAGAGCAACAGTGCTGTGGCCTGGAGCAACAAATCTGACTTTGCATGTGCAAACGCC | |
| | TTCAACAACAGCATTATTCCAGAAGACACCTTCTTCCCCAGCCCAGAAAGTTCCTGTGATGTC | |
| | AAGCTGGTCGAGAAAAGCTTTGAAACAGATACGAACCTAAACTTTCAAAACCTGTCAGTGATT | |
| | GGGTTCCGAATCCTCCTCCTGAAAGTGGCCGGGTTTAATCTGCTCATGACGCTGCGGCTGTGG | |
| | TCCAGCGGCTCCGGAGCCACGAACTTCTCTCTGTTAAAGCAAGCAGGAGACGTGGAAGAAAAC | |
| | CCCGGTCCCATGGGTACCAGCCTCCTCTGCTGGATGGCCCTGTGTCTCCTGGGGGCAGATCAC | |
| | GCAGATACTGGAGTCTCCCAGGACCCCAGACACAAGATCACAAAGAGGGGACAGAATGTAACT | |
| | TTCAGGTGTGATCCAATTTCTGAACACAACCGCCTTTATTGGTACCGACAGACCCTGGGGCAG | |
| | GGCCCAGAGTTTCTGACTTACTTCCAGAATGAAGCTCAACTAGAAAAATCAAGGCTGCTCAGT | |
| | GATCGGTTCTCTGCAGAGAGGCCTAAGGGATCTTTCTCCACCTTGGAGATCCAGCGCACAGAG | |
| | CAGGGGGACTCGGCCATGTATCTCTGTGCCAGCAGCTTAGTCTCGGGCAATGAGCAGTTCTTC | |
| | GGGCCAGGGACACGGCTCACCGTGCTAGAGGACCTGAAAAACGTGTTCCCACCCGAGGTCGCT | |
| | GTGTTTGAGCCATCAGAAGCAGAGATCTCCCACACCCAAAAGGCCACACTGGTATGCCTGGCC | |
| | ACAGGCTTCTACCCCGACCACGTGGAGCTGAGCTGGTGGGTGAATGGGAAGGAGGTGCACAGT | |
| | GGGGTCAGCACAGACCCGCAGCCCCTCAAGGAGCAGCCCGCCCTCAATGACTCCAGATACTGC | |
| | CTGAGCAGCCGCCTGAGGGTCTCGGCCACCTTCTGGCAGAACCCCCGCAACCACTTCCGCTGT | |
| | CAAGTCCAGTTCTACGGGCTCTCGGAGAATGACGAGTGGACCCAGGATAGGGCCAAACCTGTC | |
| | ACCCAGATCGTCAGCGCCGAGGCCTGGGGTAGAGCAGACTGTGGCTTCACCTCCGAGTCTTAC | |
| | CAGCAAGGGGTCCTGTCTGCCACCATCCTCTATGAGATCTTGCTAGGGAAGGCCACCTTGTAT | |
| | GCCGTGCTGGTCAGTGCCCTCGTGCTGATGGCCATGGTCAAGAGAAAGGATTCCAGAGGCTAG | |
| 64 | MASAPISMLAMLFTLSGLRAQSVAQPEDQVNVAEGNPLTVKCTYSVSGNPYLFWYVQYPNRGL | TCR D full |
| | QFLLKYITGDNLVKGSYGFEAEFNKSQTSFHLKKPSALVSDSALYFCAVRAPTSGTYKYIFGT | construct aa |
| | GTRLKVLANIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGFNLLMTLRLWSSGSGATNFSLLKQAGDVEENPGPMGTSLLCWMALCLLGADH | |
| | ADTGVSQDPRHKITKRGQNVTFRCDPISEHNRLYWYRQTLGQGPEFLTYFQNEAQLEKSRLLS | |
| | DRFSAERPKGSFSTLEIQRTEQGDSAMYLCASSLVSGNEQFFGPGTRLTVLEDLKNVFPPEVA | |
| | VFEPSEAEISHTQKATLVCLATGFYPDHVELSWWVNGKEVHSGVSTDPQPLKEQPALNDSRYC | |
| | LSSRLRVSATFWQNPRNHFRCQVQFYGLSENDEWTQDRAKPVTQIVSAEAWGRADCGFTSESY | |
| | QQGVLSATILYEILLGKATLYAVLVSALVLMAMVKRKDSRG | |
| 65 | AVRGPTSGTYKYI | TCR E Vα |
| | | CDR-3 |
| 66 | AQSVAQPEDQVNVAEGNPLTVKCTYSVSGNPYLFWYVQYPNRGLQFLLKYITGDNLVKGSYGF | TCR E Vα |
| | EAEFNKSQTSFHLKKPSALVSDSALYFCAVRGPTSGTYKYIFGTGTRLKVLAN | (aa) |
| 67 | MASAPISMLAMLFTLSGLRAQSVAQPEDQVNVAEGNPLTVKCTYSVSGNPYLFWYVQYPNRGL | TCR E Vα + |
| | QFLLKYITGDNLVKGSYGFEAEFNKSQTSFHLKKPSALVSDSALYFCAVRGPTSGTYKYIFGT | ss (aa) |
| | GTRLKVLAN | |
| 68 | MASAPISMLAMLFTLSGLRAQSVAQPEDQVNVAEGNPLTVKCTYSVSGNPYLFWYVQYPNRGL | TCR E Vα + |
| | QFLLKYITGDNLVKGSYGFEAEFNKSQTSFHLKKPSALVSDSALYFCAVRGPTSGTYKYIFGT | Cα (aa) |
| | GTRLKVLANIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGFNLLMTLRLWSS | |
| 69 | ATGGCCTCTGCACCCATCTCGATGCTTGCGATGCTCTTCACATTGAGTGGGCTGAGAGCTCAG | TCR E Vα |
| | TCAGTGGCTCAGCCGGAAGATCAGGTCAACGTTGCTGAAGGGAATCCTCTGACTGTGAAATGC | (nt) |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGGGCCTACCTCAGGAACCTACAAATACATCTTTGGAACA | |
| | GGCACCAGGCTGAAGGTTTTAGCAAAT | |
| 70 | ATGGCCTCTGCACCCATCTCGATGCTTGCGATGCTCTTCACATTGAGTGGGCTGAGAGCTCAG | TCR E Vα + |
| | TCAGTGGCTCAGCCGGAAGATCAGGTCAACGTTGCTGAAGGGAATCCTCTGACTGTGAAATGC | Cα (nt) |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGGGCCTACCTCAGGAACCTACAAATACATCTTTGGAACA | |
| | GGCACCAGGCTGAAGGTTTTAGCAAATATCCAGAACCCTGACCCTGCCGTGTACCAGCTGAGA | |
| | GACTCTAAATCCAGTGACAAGTCTGTCTGCCTATTCACCGATTTTGATTCTCAAACAAATGTG | |
| | TCACAAAGTAAGGATTCTGATGTGTATATCACAGACAAAACTGTGCTAGACATGAGGTCTATG | |
| | GACTTCAAGAGCAACAGTGCTGTGGCCTGGAGCAACAAATCTGACTTTGCATGTGCAAACGCC | |
| | TTCAACAACAGCATTATTCCAGAAGACACCTTCTTCCCCAGCCCAGAAAGTTCCTGTGATGTC | |
| | AAGCTGGTCGAGAAAAGCTTTGAAACAGATACGAACCTAAACTTTCAAAACCTGTCAGTGATT | |
| | GGGTTCCGAATCCTCCTCCTGAAAGTGGCCGGGTTTAATCTGCTCATGACGCTGCGGCTGTGG | |
| | TCCAGC | |
| 71 | MGTSLLCWMALCLLGADHADTGVSQDPRHKITKRGQNVTFRCDPISEHNRLYWYRQTLGQGPE | TCR E Vβ + |
| | FLTYFQNEAQLEKSRLLSDRFSAERPKGSFSTLEIQRTEQGDSAMYLCASSLVSGNEQFFGPG | Cβ (aa) |
| | TRLTVLEDLNKVFPPEVAVFEPSEAEISHTQKATLVCLATGFFPDHVELSWWVNGKEVHSGVS | |
| | TDPQPLKEQPALNDSRYCLSSRLRVSATFWQNPRNHFRCQVQFYGLSENDEWTQDRAKPVTQI | |
| | VSAEAWGRADCGFTSVSYQQGVLSATILYEILLGKATLYAVLVSALVLMAMVKRKDF | |
| 72 | ATGGGTACCAGCCTCCTCTGCTGGATGGCCCTGTGTCTCCTGGGGGCAGATCACGCAGATACT | TCR E Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGTAAGCGGAAATGAGCAGTTCTTCGGGCCAGGG | |
| | ACACGGCTCACCGTGCTAGAG | |
| 73 | ATGGGTACCAGCCTCCTCTGCTGGATGGCCCTGTGTCTCCTGGGGGCAGATCACGCAGATACT | TCR E Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGTAAGCGGAAATGAGCAGTTCTTCGGGCCAGGG | |
| | ACACGGCTCACCGTGCTAGAGGACCTGAACAAGGTGTTCCCACCCGAGGTCGCTGTGTTTGAG | |
| | CCATCAGAAGCAGAGATCTCCCACACCCAAAAGGCCACACTGGTGTGCCTGGCCACAGGCTTC | |
| | TTCCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCCGTCACCCAGATC | |
| | GTCAGCGCCGAGGCCTGGGGTAGAGCAGACTGTGGCTTTACCTCGGTGTCCTACCAGCAAGGG | |
| | GTCCTGTCTGCCACCATCCTCTATGAGATCCTGCTAGGGAAGGCCACCCTGTATGCTGTGCTG | |
| | GTCAGCGCCCTTGTGTTGATGGCCATGGTCAAGAGAAAGGATTTCTGA | |
| 74 | ATGGCCTCTGCACCCATCTCGATGCTTGCGATGCTCTTCACATTGAGTGGGCTGAGAGCTCAG | TCR E full |
| | TCAGTGGCTCAGCCGGAAGATCAGGTCAACGTTGCTGAAGGGAATCCTCTGACTGTGAAATGC | construct nt |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGGGCCTACCTCAGGAACCTACAAATACATCTTTGGAACA | |
| | GGCACCAGGCTGAAGGTTTTAGCAAATATCCAGAACCCTGACCCTGCCGTGTACCAGCTGAGA | |
| | GACTCTAAATCCAGTGACAAGTCTGTCTGCCTATTCACCGATTTTGATTCTCAAACAAATGTG | |
| | TCACAAAGTAAGGATTCTGATGTGTATATCACAGACAAAACTGTGCTAGACATGAGGTCTATG | |
| | GACTTCAAGAGCAACAGTGCTGTGGCCTGGAGCAACAAATCTGACTTTGCATGTGCAAACGCC | |
| | TTCAACAACAGCATTATTCCAGAAGACACCTTCTTCCCCAGCCCAGAAAGTTCCTGTGATGTC | |
| | AAGCTGGTCGAGAAAAGCTTTGAAACAGATACGAACCTAAACTTTCAAAACCTGTCAGTGATT | |
| | GGGTTCCGAATCCTCCTCCTGAAAGTGGCCGGGTTTAATCTGCTCATGACGCTGCGGCTGTGG | |
| | TCCAGCGGCTCCGGAGCCACGAACTTCTCTCTGTTAAAGCAAGCAGGAGACGTGGAAGAAAAC | |
| | CCCGGTCCCATGGGTACCAGCCTCCTCTGCTGGATGGCCCTGTGTCTCCTGGGGGCAGATCAC | |
| | GCAGATACTGGAGTCTCCCAGGACCCCAGACACAAGATCACAAAGAGGGGACAGAATGTAACT | |
| | TTCAGGTGTGATCCAATTTCTGAACACAACCGCCTTTATTGGTACCGACAGACCCTGGGGCAG | |
| | GGCCCAGAGTTTCTGACTTACTTCCAGAATGAAGCTCAACTAGAAAAATCAAGGCTGCTCAGT | |
| | GATCGGTTCTCTGCAGAGAGGCCTAAGGGATCTTTCTCCACCTTGGAGATCCAGCGCACAGAG | |
| | CAGGGGGACTCGGCCATGTATCTCTGTGCCAGCAGCTTAGTAAGCGGAAATGAGCAGTTCTTC | |
| | GGGCCAGGGACACGGCTCACCGTGCTAGAGGACCTGAACAAGGTGTTCCCACCCGAGGTCGCT | |
| | GTGTTTGAGCCATCAGAAGCAGAGATCTCCCACACCCAAAAGGCCACACTGGTGTGCCTGGCC | |
| | ACAGGCTTCTTCCCCGACCACGTGGAGCTGAGCTGGTGGGTGAATGGGAAGGAGGTGCACAGT | |
| | GGGGTCAGCACAGACCCGCAGCCCCTCAAGGAGCAGCCCGCCCTCAATGACTCCAGATACTGC | |
| | CTGAGCAGCCGCCTGAGGGTCTCGGCCACCTTCTGGCAGAACCCCCGCAACCACTTCCGCTGT | |
| | CAAGTCCAGTTCTACGGGCTCTCGGAGAATGACGAGTGGACCCAGGATAGGGCCAAACCCGTC | |
| | ACCCAGATCGTCAGCGCCGAGGCCTGGGGTAGAGCAGACTGTGGCTTTACCTCGGTGTCCTAC | |
| | CAGCAAGGGGTCCTGTCTGCCACCATCCTCTATGAGATCCTGCTAGGGAAGGCCACCCTGTAT | |
| | GCTGTGCTGGTCAGCGCCCTTGTGTTGATGGCCATGGTCAAGAGAAAGGATTTCTGA | |
| 75 | MASAPISMLAMLFTLSGLRAQSVAQPEDQVNVAEGNPLTVKCTYSVSGNPYLFWYVQYPNRGL | TCR E full |
| | QFLLKYITGDNLVKGSYGFEAEFNKSQTSFHLKKPSALVSDSALYFCAVRGPTSGTYKYIFGT | construct aa |
| | GTRLKVLANIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGFNLLMTLRLWSSGSGATNFSLLKQAGDVEENPGPMGTSLLCWMALCLLGADH | |
| | ADTGVSQDPRHKITKRGQNVTFRCDPISEHNRLYWYRQTLGQGPEFLTYFQNEAQLEKSRLLS | |
| | DRFSAERPKGSFSTLEIQRTEQGDSAMYLCASSLVSGNEQFFGPGTRLTVLEDLNKVFPPEVA | |
| | VFEPSEAEISHTQKATLVCLATGFFPDHVELSWWVNGKEVHSGVSTDPQPLKEQPALNDSRYC | |
| | LSSRLRVSATFWQNPRNHFRCQVQFYGLSENDEWTQDRAKPVTQIVSAEAWGRADCGFTSVSY | |
| | QQGVLSATILYEILLGKATLYAVLVSALVLMAMVKRKDF | |
| 76 | DSASNY | TCR F, H, N, |
| | | O, Q Vα |
| | | CDR-1 |
| 77 | IRSNVGE | TCR F, H, N, |
| | | O, Q Vα |
| | | CDR-2 |
| 78 | AAHLTGGGNKLT | TCR F Vα |
| | | CDR-3 |
| 79 | GENVEQHPSTLSVQEGDSAVIKCTYSDSASNYFPWYKQELGKRPQLIIDIRSNVGEKKDQRIA | TCR F Vα |
| | VTLNKTAKHFSLHITETQPEDSAVYFCAAHLTGGGNKLTFGTGTQLKVELN | (aa) |
| 80 | MSIRAVFIFLWLQLDLVNGENVEQHPSTLSVQEGDSAVIKCTYSDSASNYFPWYKQELGKRPQ | TCR F Vα + |
| | LIIDIRSNVGEKKDQRIAVTLNKTAKHFSLHITETQPEDSAVYFCAAHLTGGGNKLTFGTGTQ | ss (aa) |
| | LKVELN | |
| 81 | MSIRAVFIFLWLQLDLVNGENVEQHPSTLSVQEGDSAVIKCTYSDSASNYFPWYKQELGKRPQ | TCR F Vα + |
| | LIIDIRSNVGEKKDQRIAVTLNKTAKHFSLHITETQPEDSAVYFCAAHLTGGGNKLTFGTGTQ | Cα (aa) |
| | LKVELNIQKPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGFR | |
| | ILLLKVAGFNLLMTLRLWSS | |
| 82 | ATGTCCATTCGAGCTGTATTTATATTCCTGTGGCTGCAGCTGGACTTGGTGAATGGAGAGAAT | TCR F Vα |
| | GTGGAGCAGCATCCTTCAACCCTGAGTGTCCAGGAGGGAGACAGCGCTGTTATCAAGTGTACT | (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCACATCTGACGGGAGGAGGAAACAAACTCACCTTTGGGACAGGCACTCAG | |
| | CTAAAAGTGGAACTCAAT | |
| 83 | ATGTCCATTCGAGCTGTATTTATATTCCTGTGGCTGCAGCTGGACTTGGTGAATGGAGAGAAT | TCR F Vα + |
| | GTGGAGCAGCATCCTTCAACCCTGAGTGTCCAGGAGGGAGACAGCGCTGTTATCAAGTGTACT | Cα (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCACATCTGACGGGAGGAGGAAACAAACTCACCTTTGGGACAGGCACTCAG | |
| | CTAAAAGTGGAACTCAATATCCAGAAGCCTGACCCTGCCGTGTACCAGCTGAGAGACTCTAAA | |
| | TCCAGTGACAAGTCTGTCTGCCTATTCACCGATTTTGATTCTCAAACAAATGTGTCACAAAGT | |
| | AAGGATTCTGATGTGTATATCACAGACAAAACTGTGCTAGACATGAGGTCTATGGACTTCAAG | |
| | AGCAACAGTGCTGTGGCCTGGAGCAACAAATCTGACTTTGCATGTGCAAACGCCTTCAACAAC | |
| | AGCATTATTCCAGAAGACACCTTCTTCCCCAGCCCAGAAAGTTCCTGTGATGTCAAGCTGGTC | |
| | GAGAAAAGCTTTGAAACAGATACGAACCTAAACTTTCAAAACCTGTCAGTGATTGGGTTCCGA | |
| 84 | ASSSRAGGETQY | TCR F Vβ |
| | | CDR-3 |
| 85 | DTGVSQDPRHKITKRGQNVTFRCDPISEHNRLYWYRQTLGQGPEFLTYFQNEAQLEKSRLLSD | TCR F Vβ |
| | RFSAERPKGSFSTLEIQRTEQGDSAMYLCASSSRAGGETQYFGPGTRLLVLE | (aa) |
| 86 | MGTSLLCWMALCLLGADHADTGVSQDPRHKITKRGQNVTFRCDPISEHNRLYWYRQTLGQGPE | TCR F Vβ + |
| | FLTYFQNEAQLEKSRLLSDRFSAERPKGSESTLEIQRTEQGDSAMYLCASSSRAGGETQYFGP | ss (aa) |
| | GTRLLVLE | |
| 87 | MGTSLLCWMALCLLGADHADTGVSQDPRHKITKRGQNVTFRCDPISEHNRLYWYRQTLGQGPE | TCR F Vβ + |
| | FLTYFQNEAQLEKSRLLSDRFSAERPKGSESTLEIQRTEQGDSAMYLCASSSRAGGETQYFGP | Cβ (aa) |
| | GTRLLVLEDLKNVFPPEVAVFEPSEAEISHTQKATLVCLATGFYPDHVELSWWVNGKEVHSGV | |
| | STDPQPLKEQPALNDSRYCLSSRLRVSATFWQNPRNHERCQVQFYGLSENDEWTQDRAKPVTQ | |
| | IVSAEAWGRADCGFTSESYQQGVLSATILYEILLGKATLYAVLVSALVLMAMVKRKDSRG* | |
| 88 | ATGGGTACCAGCCTCCTCTGCTGGATGGCCCTGTGTCTCCTGGGGGCAGATCACGCAGATACT | TCR F Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTCCCGGGCCGGAGGGGAGACCCAGTACTTCGGGCCA | |
| | GGCACGCGGCTCCTGGTGCTCGAG | |
| 89 | ATGGGTACCAGCCTCCTCTGCTGGATGGCCCTGTGTCTCCTGGGGGCAGATCACGCAGATACT | TCR F Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTCCCGGGCCGGAGGGGAGACCCAGTACTTCGGGCCA | |
| | GGCACGCGGCTCCTGGTGCTCGAGGACCTGAAAAACGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 90 | ATGTCCATTCGAGCTGTATTTATATTCCTGTGGCTGCAGCTGGACTTGGTGAATGGAGAGAAT | TCR F full |
| | GTGGAGCAGCATCCTTCAACCCTGAGTGTCCAGGAGGGAGACAGCGCTGTTATCAAGTGTACT | construct nt |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCACATCTGACGGGAGGAGGAAACAAACTCACCTTTGGGACAGGCACTCAG | |
| | CTAAAAGTGGAACTCAATATCCAGAAGCCTGACCCTGCCGTGTACCAGCTGAGAGACTCTAAA | |
| | TCCAGTGACAAGTCTGTCTGCCTATTCACCGATTTTGATTCTCAAACAAATGTGTCACAAAGT | |
| | AAGGATTCTGATGTGTATATCACAGACAAAACTGTGCTAGACATGAGGTCTATGGACTTCAAG | |
| | AGCAACAGTGCTGTGGCCTGGAGCAACAAATCTGACTTTGCATGTGCAAACGCCTTCAACAAC | |
| | AGCATTATTCCAGAAGACACCTTCTTCCCCAGCCCAGAAAGTTCCTGTGATGTCAAGCTGGTC | |
| | GAGAAAAGCTTTGAAACAGATACGAACCTAAACTTTCAAAACCTGTCAGTGATTGGGTTCCGA | |
| | ATCCTCCTCCTGAAAGTGGCCGGGTTTAATCTGCTCATGACGCTGCGGCTGTGGTCCAGCGGC | |
| | TCCGGAGCCACGAACTTCTCTCTGTTAAAGCAAGCAGGAGACGTGGAAGAAAACCCCGGTCCC | |
| | ATGGGTACCAGCCTCCTCTGCTGGATGGCCCTGTGTCTCCTGGGGGCAGATCACGCAGATACT | |
| | GGAGTCTCCCAGGACCCCAGACACAAGATCACAAAGAGGGGACAGAATGTAACTTTCAGGTGT | |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTCCCGGGCCGGAGGGGAGACCCAGTACTTCGGGCCA | |
| | GGCACGCGGCTCCTGGTGCTCGAGGACCTGAAAAACGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 91 | MSIRAVFIFLWLQLDLVNGENVEQHPSTLSVQEGDSAVIKCTYSDSASNYFPWYKQELGKRPQ | TCR F full |
| | LIIDIRSNVGEKKDQRIAVTLNKTAKHFSLHITETQPEDSAVYFCAAHLTGGGNKLTFGTGTQ | construct aa |
| | LKVELNIQKPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGFR | |
| | ILLLKVAGFNLLMTLRLWSSGSGATNFSLLKQAGDVEENPGPMGTSLLCWMALCLLGADHADT | |
| | GVSQDPRHKITKRGQNVTFRCDPISEHNRLYWYRQTLGQGPEFLTYFQNEAQLEKSRLLSDRF | |
| | SAERPKGSFSTLEIQRTEQGDSAMYLCASSSRAGGETQYFGPGTRLLVLEDLKNVEPPEVAVF | |
| | EPSEAEISHTQKATLVCLATGFYPDHVELSWWVNGKEVHSGVSTDPQPLKEQPALNDSRYCLS | |
| | SRLRVSATFWQNPRNHFRCQVQFYGLSENDEWTQDRAKPVTQIVSAEAWGRADCGFTSESYQQ | |
| | GVLSATILYEILLGKATLYAVLVSALVLMAMVKRKDSRG* | |
| 92 | AGTGANNLF | TCR G Vα |
| | | CDR-3 |
| 93 | SQQGEEDPQALSIQEGENATMNCSYKTSINNLQWYRQNSGRGLVHLILIRSNEREKHSGRLRV | TCR G Vα |
| | TLDTSKKSSSLLITASRAADTASYFCAGTGANNLFFGTGTRLTVIP | (aa) |
| 94 | METLLGVSLVILWLQLARVNSQQGEEDPQALSIQEGENATMNCSYKTSINNLQWYRQNSGRGL | TCR G Vα + |
| | VHLILIRSNEREKHSGRLRVTLDTSKKSSSLLITASRAADTASYFCAGTGANNLFFGTGTRLT | ss (aa) |
| | VIP | |
| 95 | METLLGVSLVILWLQLARVNSQQGEEDPQALSIQEGENATMNCSYKTSINNLQWYRQNSGRGL | TCR G Vα + |
| | VHLILIRSNEREKHSGRLRVTLDTSKKSSSLLITASRAADTASYFCAGTGANNLFFGTGTRLT | Cα (aa) |
| | VIPYIQKPDPAVYQLRDSKSSDKSVCLFTDFDSQTNVSQSKDSDVYITDKTVLDMRSMDFKSN | |
| | SAVAWSNKSDFACANAFNNSIIPEDTFFPSPESSCDVKLVEKSFETDTNLNFQNLSVIGFRIL | |
| | LLKVAGFNLLMTLRLWSS | |
| 96 | ATGGAAACTCTCCTGGGAGTGTCTTTGGTGATTCTATGGCTTCAACTGGCTAGGGTGAACAGT | TCR G Vα |
| | CAACAGGGAGAAGAGGATCCTCAGGCCTTGAGCATCCAGGAGGGTGAAAATGCCACCATGAAC | (nt) |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTGGAACTGGGGCAAACAACCTCTTCTTTGGGACTGGAACGAGACTCACC | |
| | GTTATTCCCTAT | |
| 97 | ATGGAAACTCTCCTGGGAGTGTCTTTGGTGATTCTATGGCTTCAACTGGCTAGGGTGAACAGT | TCR G Vα + |
| | CAACAGGGAGAAGAGGATCCTCAGGCCTTGAGCATCCAGGAGGGTGAAAATGCCACCATGAAC | Cα (nt) |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTGGAACTGGGGCAAACAACCTCTTCTTTGGGACTGGAACGAGACTCACC | |
| | GTTATTCCCTATATCCAGAAGCCTGACCCTGCCGTGTACCAGCTGAGAGACTCTAAATCCAGT | |
| | GACAAGTCTGTCTGCCTATTCACCGATTTTGATTCTCAAACAAATGTGTCACAAAGTAAGGAT | |
| | TCTGATGTGTATATCACAGACAAAACTGTGCTAGACATGAGGTCTATGGACTTCAAGAGCAAC | |
| | AGTGCTGTGGCCTGGAGCAACAAATCTGACTTTGCATGTGCAAACGCCTTCAACAACAGCATT | |
| | ATTCCAGAAGACACCTTCTTCCCCAGCCCAGAAAGTTCCTGTGATGTCAAGCTGGTCGAGAAA | |
| | AGCTTTGAAACAGATACGAACCTAAACTTTCAAAACCTGTCAGTGATTGGGTTCCGAATCCTC | |
| | CTCCTGAAAGTGGCCGGGTTTAATCTGCTCATGACGCTGCGGCTGTGGTCCAGC | |
| 98 | ASSLIRGETQY | TCR G Vβ |
| | | CDR-3 |
| 99 | DTGVSQDPRHKITKRGQNVTFRCDPISEHNRLYWYRQTLGQGPEFLTYFQNEAQLEKSRLLSD | TCR G Vβ |
| | RFSAERPKGSFSTLEIQRTEQGDSAMYLCASSLIRGETQYFGPGTRLLVLE | (aa) |
| 100 | MGTSLLCWMALCLLGADHADTGVSQDPRHKITKRGQNVTFRCDPISEHNRLYWYRQTLGQGPE | TCR G Vβ + |
| | FLTYFQNEAQLEKSRLLSDRFSAERPKGSFSTLEIQRTEQGDSAMYLCASSLIRGETQYFGPG | ss (aa) |
| | TRLLVLE | |
| 101 | MGTSLLCWMALCLLGADHADTGVSQDPRHKITKRGQNVTFRCDPISEHNRLYWYRQTLGQGPE | TCR G Vβ + |
| | FLTYFQNEAQLEKSRLLSDRFSAERPKGSESTLEIQRTEQGDSAMYLCASSLIRGETQYFGPG | Cβ (aa) |
| | TRLLVLEDLKNVFPPEVAVFEPSEAEISHTQKATLVCLATGFYPDHVELSWWVNGKEVHSGVS | |
| | TDPQPLKEQPALNDSRYCLSSRLRVSATFWQNPRNHFRCQVQFYGLSENDEWTQDRAKPVTQI | |
| | VSAEAWGRADCGFTSESYQQGVLSATILYEILLGKATLYAVLVSALVLMAMVKRKDSRG | |
| 102 | ATGGGTACCAGCCTCCTCTGCTGGATGGCCCTGTGTCTCCTGGGGGCAGATCACGCAGATACT | TCR G Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAATCAGGGGAGAGACCCAGTACTTCGGGCCAGGC | |
| | ACGCGGCTCCTGGTGCTCGAG | |
| 103 | ATGGGTACCAGCCTCCTCTGCTGGATGGCCCTGTGTCTCCTGGGGGCAGATCACGCAGATACT | TCR G Vβ+ |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAATCAGGGGAGAGACCCAGTACTTCGGGCCAGGC | |
| | ACGCGGCTCCTGGTGCTCGAGGACCTGAAAAACGTGTTCCCACCCGAGGTCGCTGTGTTTGAG | |
| | CCATCAGAAGCAGAGATCTCCCACACCCAAAAGGCCACACTGGTATGCCTGGCCACAGGCTTC | |
| | TACCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCTGTCACCCAGATC | |
| | GTCAGCGCCGAGGCCTGGGGTAGAGCAGACTGTGGCTTCACCTCCGAGTCTTACCAGCAAGGG | |
| | GTCCTGTCTGCCACCATCCTCTATGAGATCTTGCTAGGGAAGGCCACCTTGTATGCCGTGCTG | |
| | GTCAGTGCCCTCGTGCTGATGGCCATGGTCAAGAGAAAGGATTCCAGAGGCTAG | |
| 104 | ATGGAAACTCTCCTGGGAGTGTCTTTGGTGATTCTATGGCTTCAACTGGCTAGGGTGAACAGT | TCR G full |
| | CAACAGGGAGAAGAGGATCCTCAGGCCTTGAGCATCCAGGAGGGTGAAAATGCCACCATGAAC | construct nt |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTGGAACTGGGGCAAACAACCTCTTCTTTGGGACTGGAACGAGACTCACC | |
| | GTTATTCCCTATATCCAGAAGCCTGACCCTGCCGTGTACCAGCTGAGAGACTCTAAATCCAGT | |
| | GACAAGTCTGTCTGCCTATTCACCGATTTTGATTCTCAAACAAATGTGTCACAAAGTAAGGAT | |
| | TCTGATGTGTATATCACAGACAAAACTGTGCTAGACATGAGGTCTATGGACTTCAAGAGCAAC | |
| | AGTGCTGTGGCCTGGAGCAACAAATCTGACTTTGCATGTGCAAACGCCTTCAACAACAGCATT | |
| | ATTCCAGAAGACACCTTCTTCCCCAGCCCAGAAAGTTCCTGTGATGTCAAGCTGGTCGAGAAA | |
| | AGCTTTGAAACAGATACGAACCTAAACTTTCAAAACCTGTCAGTGATTGGGTTCCGAATCCTC | |
| | CTCCTGAAAGTGGCCGGGTTTAATCTGCTCATGACGCTGCGGCTGTGGTCCAGCGGCTCCGGA | |
| | GCCACGAACTTCTCTCTGTTAAAGCAAGCAGGAGACGTGGAAGAAAACCCCGGTCCCATGGGT | |
| | ACCAGCCTCCTCTGCTGGATGGCCCTGTGTCTCCTGGGGGCAGATCACGCAGATACTGGAGTC | |
| | TCCCAGGACCCCAGACACAAGATCACAAAGAGGGGACAGAATGTAACTTTCAGGTGTGATCCA | |
| | ATTTCTGAACACAACCGCCTTTATTGGTACCGACAGACCCTGGGGCAGGGCCCAGAGTTTCTG | |
| | ACTTACTTCCAGAATGAAGCTCAACTAGAAAAATCAAGGCTGCTCAGTGATCGGTTCTCTGCA | |
| | GAGAGGCCTAAGGGATCTTTCTCCACCTTGGAGATCCAGCGCACAGAGCAGGGGGACTCGGCC | |
| | ATGTATCTCTGTGCCAGCAGCTTAATCAGGGGAGAGACCCAGTACTTCGGGCCAGGCACGCGG | |
| | CTCCTGGTGCTCGAGGACCTGAAAAACGTGTTCCCACCCGAGGTCGCTGTGTTTGAGCCATCA | |
| | GAAGCAGAGATCTCCCACACCCAAAAGGCCACACTGGTATGCCTGGCCACAGGCTTCTACCCC | |
| | GACCACGTGGAGCTGAGCTGGTGGGTGAATGGGAAGGAGGTGCACAGTGGGGTCAGCACAGAC | |
| | CCGCAGCCCCTCAAGGAGCAGCCCGCCCTCAATGACTCCAGATACTGCCTGAGCAGCCGCCTG | |
| | AGGGTCTCGGCCACCTTCTGGCAGAACCCCCGCAACCACTTCCGCTGTCAAGTCCAGTTCTAC | |
| | GGGCTCTCGGAGAATGACGAGTGGACCCAGGATAGGGCCAAACCTGTCACCCAGATCGTCAGC | |
| | GCCGAGGCCTGGGGTAGAGCAGACTGTGGCTTCACCTCCGAGTCTTACCAGCAAGGGGTCCTG | |
| | TCTGCCACCATCCTCTATGAGATCTTGCTAGGGAAGGCCACCTTGTATGCCGTGCTGGTCAGT | |
| | GCCCTCGTGCTGATGGCCATGGTCAAGAGAAAGGATTCCAGAGGCTAG | |
| 105 | METLLGVSLVILWLQLARVNSQQGEEDPQALSIQEGENATMNCSYKTSINNLQWYRQNSGRGL | TCR G full |
| | VHLILIRSNEREKHSGRLRVTLDTSKKSSSLLITASRAADTASYFCAGTGANNLFFGTGTRLT | construct aa |
| | VIPYIQKPDPAVYQLRDSKSSDKSVCLFTDFDSQTNVSQSKDSDVYITDKTVLDMRSMDFKSN | |
| | SAVAWSNKSDFACANAFNNSIIPEDTFFPSPESSCDVKLVEKSFETDTNLNFQNLSVIGFRIL | |
| | LLKVAGFNLLMTLRLWSSGSGATNFSLLKQAGDVEENPGPMGTSLLCWMALCLLGADHADTGV | |
| | SQDPRHKITKRGQNVTFRCDPISEHNRLYWYRQTLGQGPEFLTYFQNEAQLEKSRLLSDRFSA | |
| | ERPKGSFSTLEIQRTEQGDSAMYLCASSLIRGETQYFGPGTRLLVLEDLKNVFPPEVAVFEPS | |
| | EAEISHTQKATLVCLATGFYPDHVELSWWVNGKEVHSGVSTDPQPLKEQPALNDSRYCLSSRL | |
| | RVSATFWQNPRNHFRCQVQFYGLSENDEWTQDRAKPVTQIVSAEAWGRADCGFTSESYQQGVL | |
| | SATILYEILLGKATLYAVLVSALVLMAMVKRKDSRG | |
| 106 | AAPLAGGGADGLT | TCR H Vα |
| | | CDR-3 |
| 107 | GENVEQHPSTLSVQEGDSAVIKCTYSDSASNYFPWYKQELGKRPQLIIDIRSNVGEKKDQRIA | TCR H Vα |
| | VTLNKTAKHFSLHITETQPEDSAVYFCAAPLAGGGADGLTFGKGTHLIIQPY | (aa) |
| 108 | MSIRAVFIFLWLQLDLVNGENVEQHPSTLSVQEGDSAVIKCTYSDSASNYFPWYKQELGKRPQ | TCR H Vα + |
| | LIIDIRSNVGEKKDQRIAVTLNKTAKHFSLHITETQPEDSAVYFCAAPLAGGGADGLTFGKGT | ss (aa) |
| | HLIIQPY | |
| 109 | MSIRAVFIFLWLQLDLVNGENVEQHPSTLSVQEGDSAVIKCTYSDSASNYFPWYKQELGKRPQ | TCR H Vα + |
| | LIIDIRSNVGEKKDQRIAVTLNKTAKHFSLHITETQPEDSAVYFCAAPLAGGGADGLTFGKGT | Cα (aa) |
| | HLIIQPYIQNPDPAVYQLRDSKSSDKSVCLFTDFDSQTNVSQSKDSDVYITDKTVLDMRSMDF | |
| | KSNSAVAWSNKSDFACANAFNNSIIPEDTFFPSPESSCDVKLVEKSFETDTNLNFQNLSVIGF | |
| | RILLLKVAGFNLLMTLRLWSS | |
| 110 | ATGTCCATTCGAGCTGTATTTATATTCCTGTGGCTGCAGCTGGACTTGGTGAATGGAGAGAAT | TCR H Vα |
| | GTGGAGCAGCATCCTTCAACCCTGAGTGTCCAGGAGGGAGACAGCGCTGTTATCAAGTGTACT | (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCACCTTTGGCAGGAGGAGGTGCTGACGGACTCACCTTTGGCAAAGGGACT | |
| | CATCTAATCATCCAGCCCTAT | |
| 111 | ATGTCCATTCGAGCTGTATTTATATTCCTGTGGCTGCAGCTGGACTTGGTGAATGGAGAGAAT | TCR H Vα + |
| | GTGGAGCAGCATCCTTCAACCCTGAGTGTCCAGGAGGGAGACAGCGCTGTTATCAAGTGTACT | Cα (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCACCTTTGGCAGGAGGAGGTGCTGACGGACTCACCTTTGGCAAAGGGACT | |
| | CATCTAATCATCCAGCCCTATATCCAGAACCCTGACCCTGCCGTGTACCAGCTGAGAGACTCT | |
| | AAATCCAGTGACAAGTCTGTCTGCCTATTCACCGATTTTGATTCTCAAACAAATGTGTCACAA | |
| | AGTAAGGATTCTGATGTGTATATCACAGACAAAACTGTGCTAGACATGAGGTCTATGGACTTC | |
| | AAGAGCAACAGTGCTGTGGCCTGGAGCAACAAATCTGACTTTGCATGTGCAAACGCCTTCAAC | |
| | AACAGCATTATTCCAGAAGACACCTTCTTCCCCAGCCCAGAAAGTTCCTGTGATGTCAAGCTG | |
| | GTCGAGAAAAGCTTTGAAACAGATACGAACCTAAACTTTCAAAACCTGTCAGTGATTGGGTTC | |
| | CGAATCCTCCTCCTGAAAGTGGCCGGGTTTAATCTGCTCATGACGCTGCGGCTGTGGTCCAGC | |
| 112 | ASSTTLITGYT | TCR H Vβ |
| | | CDR-3 |
| 113 | DTGVSQDPRHKITKRGQNVTFRCDPISEHNRLYWYRQTLGQGPEFLTYFQNEAQLEKSRLLSD | TCR H Vβ |
| | RFSAERPKGSFSTLEIQRTEQGDSAMYLCASSTTLITGYTFGSGTRLTVVE | (aa) |
| 114 | MGTSLLCWMALCLLGADHADTGVSQDPRHKITKRGQNVTFRCDPISEHNRLYWYRQTLGQGPE | TCR H Vβ + |
| | FLTYFQNEAQLEKSRLLSDRFSAERPKGSFSTLEIQRTEQGDSAMYLCASSTTLITGYTFGSG | ss (aa) |
| | TRLTVVE | |
| 115 | MGTSLLCWMALCLLGADHADTGVSQDPRHKITKRGQNVTFRCDPISEHNRLYWYRQTLGQGPE | TCR H Vβ + |
| | FLTYFQNEAQLEKSRLLSDRFSAERPKGSFSTLEIQRTEQGDSAMYLCASSTTLITGYTFGSG | Cβ (aa) |
| | TRLTVVEDLNKVFPPEVAVFEPSEAEISHTQKATLVCLATGFYPDHVELSWWVNGKEVHSGVS | |
| | TDPQPLKEQPALNDSRYCLSSRLRVSATFWQNPRNHFRCQVQFYGLSENDEWTQDRAKPVTQI | |
| | VSAEAWGRADCGFTSESYQQGVLSATILYEILLGKATLYAVLVSALVLMAMVKRKDSRG | |
| 116 | ATGGGTACCAGCCTCCTCTGCTGGATGGCCCTGTGTCTCCTGGGGGCAGATCACGCAGATACT | TCR H Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCACGACTCTTATAACGGGCTACACCTTCGGTTCGGGG | |
| | ACCAGGTTAACCGTTGTAGAG | |
| 117 | ATGGGTACCAGCCTCCTCTGCTGGATGGCCCTGTGTCTCCTGGGGGCAGATCACGCAGATACT | TCR H Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCACGACTCTTATAACGGGCTACACCTTCGGTTCGGGG | |
| | ACCAGGTTAACCGTTGTAGAGGACCTGAACAAGGTGTTCCCACCCGAGGTCGCTGTGTTTGAG | |
| | CCATCAGAAGCAGAGATCTCCCACACCCAAAAGGCCACACTGGTATGCCTGGCCACAGGCTTC | |
| | TACCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCTGTCACCCAGATC | |
| | GTCAGCGCCGAGGCCTGGGGTAGAGCAGACTGTGGCTTCACCTCCGAGTCTTACCAGCAAGGG | |
| | GTCCTGTCTGCCACCATCCTCTATGAGATCTTGCTAGGGAAGGCCACCTTGTATGCCGTGCTG | |
| | GTCAGTGCCCTCGTGCTGATGGCCATGGTCAAGAGAAAGGATTCCAGAGGCTAG | |
| 118 | ATGTCCATTCGAGCTGTATTTATATTCCTGTGGCTGCAGCTGGACTTGGTGAATGGAGAGAAT | TCR H full |
| | GTGGAGCAGCATCCTTCAACCCTGAGTGTCCAGGAGGGAGACAGCGCTGTTATCAAGTGTACT | construct nt |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCACCTTTGGCAGGAGGAGGTGCTGACGGACTCACCTTTGGCAAAGGGACT | |
| | CATCTAATCATCCAGCCCTATATCCAGAACCCTGACCCTGCCGTGTACCAGCTGAGAGACTCT | |
| | AAATCCAGTGACAAGTCTGTCTGCCTATTCACCGATTTTGATTCTCAAACAAATGTGTCACAA | |
| | AGTAAGGATTCTGATGTGTATATCACAGACAAAACTGTGCTAGACATGAGGTCTATGGACTTC | |
| | AAGAGCAACAGTGCTGTGGCCTGGAGCAACAAATCTGACTTTGCATGTGCAAACGCCTTCAAC | |
| | AACAGCATTATTCCAGAAGACACCTTCTTCCCCAGCCCAGAAAGTTCCTGTGATGTCAAGCTG | |
| | GTCGAGAAAAGCTTTGAAACAGATACGAACCTAAACTTTCAAAACCTGTCAGTGATTGGGTTC | |
| | CGAATCCTCCTCCTGAAAGTGGCCGGGTTTAATCTGCTCATGACGCTGCGGCTGTGGTCCAGC | |
| | GGCTCCGGAGCCACGAACTTCTCTCTGTTAAAGCAAGCAGGAGACGTGGAAGAAAACCCCGGT | |
| | CCCATGGGTACCAGCCTCCTCTGCTGGATGGCCCTGTGTCTCCTGGGGGCAGATCACGCAGAT | |
| | ACTGGAGTCTCCCAGGACCCCAGACACAAGATCACAAAGAGGGGACAGAATGTAACTTTCAGG | |
| | TGTGATCCAATTTCTGAACACAACCGCCTTTATTGGTACCGACAGACCCTGGGGCAGGGCCCA | |
| | GAGTTTCTGACTTACTTCCAGAATGAAGCTCAACTAGAAAAATCAAGGCTGCTCAGTGATCGG | |
| | TTCTCTGCAGAGAGGCCTAAGGGATCTTTCTCCACCTTGGAGATCCAGCGCACAGAGCAGGGG | |
| | GACTCGGCCATGTATCTCTGTGCCAGCAGCACGACTCTTATAACGGGCTACACCTTCGGTTCG | |
| | GGGACCAGGTTAACCGTTGTAGAGGACCTGAACAAGGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 119 | MSIRAVFIFLWLQLDLVNGENVEQHPSTLSVQEGDSAVIKCTYSDSASNYFPWYKQELGKRPQ | TCR H full |
| | LIIDIRSNVGEKKDQRIAVTLNKTAKHFSLHITETQPEDSAVYFCAAPLAGGGADGLTFGKGT | construct aa |
| | HLIIQPYIQNPDPAVYQLRDSKSSDKSVCLFTDFDSQTNVSQSKDSDVYITDKTVLDMRSMDF | |
| | KSNSAVAWSNKSDFACANAFNNSIIPEDTFFPSPESSCDVKLVEKSFETDTNLNFQNLSVIGF | |
| | RILLLKVAGFNLLMTLRLWSSGSGATNFSLLKQAGDVEENPGPMGTSLLCWMALCLLGADHAD | |
| | TGVSQDPRHKITKRGQNVTFRCDPISEHNRLYWYRQTLGQGPEFLTYFQNEAQLEKSRLLSDR | |
| | FSAERPKGSFSTLEIQRTEQGDSAMYLCASSTTLITGYTFGSGTRLTVVEDLNKVFPPEVAVF | |
| | EPSEAEISHTQKATLVCLATGFYPDHVELSWWVNGKEVHSGVSTDPQPLKEQPALNDSRYCLS | |
| | SRLRVSATFWQNPRNHFRCQVQFYGLSENDEWTQDRAKPVTQIVSAEAWGRADCGFTSESYQQ | |
| | GVLSATILYEILLGKATLYAVLVSALVLMAMVKRKDSRG | |
| 120 | AVRGTTSGTYKYI | TCR I Vα |
| | | CDR-3 |
| 121 | KQEVTQIPAALSVPEGENLVLNCSFTDSAIYNLQWFRQDPGKGLTSLLLIQSSQREQTSGRLN | TCR I Vα (aa) |
| | ASLDKSSGRSTLYIAASQPGDSATYLCAVRGTTSGTYKYIFGTGTRLKVLAN | |
| 122 | METLLGLLILWLQLQWVSSKQEVTQIPAALSVPEGENLVLNCSFTDSAIYNLQWFRQDPGKGL | TCR I Vα + ss |
| | TSLLLIQSSQREQTSGRLNASLDKSSGRSTLYIAASQPGDSATYLCAVRGTTSGTYKYIFGTG | (aa) |
| | TRLKVLAN | |
| 123 | METLLGLLILWLQLQWVSSKQEVTQIPAALSVPEGENLVLNCSFTDSAIYNLQWFRQDPGKGL | TCR I Vα + |
| | TSLLLIQSSQREQTSGRLNASLDKSSGRSTLYIAASQPGDSATYLCAVRGTTSGTYKYIFGTG | Cα (aa) |
| | TRLKVLANIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSS | |
| 124 | ATGGAGACCCTCTTGGGCCTGCTTATCCTTTGGCTGCAGCTGCAATGGGTGAGCAGCAAACAG | TCR I Vα (nt) |
| | GAGGTGACGCAGATTCCTGCAGCTCTGAGTGTCCCAGAAGGAGAAAACTTGGTTCTCAACTGC | |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTTCGTGGGACTACCTCAGGAACCTACAAATACATCTTTGGAACAGGC | |
| | ACCAGGCTGAAGGTTTTAGCAAAT | |
| 125 | ATGGAGACCCTCTTGGGCCTGCTTATCCTTTGGCTGCAGCTGCAATGGGTGAGCAGCAAACAG | TCR I Vα + |
| | GAGGTGACGCAGATTCCTGCAGCTCTGAGTGTCCCAGAAGGAGAAAACTTGGTTCTCAACTGC | Cα (nt) |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTTCGTGGGACTACCTCAGGAACCTACAAATACATCTTTGGAACAGGC | |
| | ACCAGGCTGAAGGTTTTAGCAAATATCCAGAACCCTGACCCTGCCGTGTACCAGCTGAGAGAC | |
| | TCTAAATCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGTCTATGGAC | |
| | TTCAAGAGCAACAGTGCTGTGGCCTGGAGCAACAAATCTGACTTTGCATGTGCAAACGCCTTC | |
| | AACAACAGCATTATTCCAGAAGACACCTTCTTCCCCAGCCCAGAAAGTTCCTGTGATGTCAAG | |
| | CTGGTCGAGAAAAGCTTTGAAACAGATACGAACCTAAACTTTCAAAACCTGTCAGTGATTGGG | |
| | TTCCGAATCCTCCTCCTGAAAGTGGCCGGGTTTAATCTGCTCATGACGCTGCGGCTGTGGTCC | |
| | AGC | |
| 126 | ASSFLAGETQY | TCR I Vβ |
| | | CDR-3 |
| 127 | DTGVSQDPRHKITKRGQNVTFRCDPISEHNRLYWYRQTLGQGPEFLTYFQNEAQLEKSRLLSD | TCR I Vβ (aa) |
| | RFSAERPKGSFSTLEIQRTEQGDSAMYLCASSFLAGETQYFGPGTRLLVLE | |
| 128 | MGTSLLCWMALCLLGADHADTGVSQDPRHKITKRGQNVTFRCDPISEHNRLYWYRQTLGQGPE | TCR I Vβ + ss |
| | FLTYFQNEAQLEKSRLLSDRFSAERPKGSFSTLEIQRTEQGDSAMYLCASSFLAGETQYFGPG | (aa) |
| | TRLLVLE | |
| 129 | MGTSLLCWMALCLLGADHADTGVSQDPRHKITKRGQNVTFRCDPISEHNRLYWYRQTLGQGPE | TCR I Vβ + |
| | FLTYFQNEAQLEKSRLLSDRFSAERPKGSFSTLEIQRTEQGDSAMYLCASSFLAGETQYFGPG | Cβ (aa) |
| | TRLLVLEDLKNVFPPEVAVFEPSEAEISHTQKATLVCLATGFYPDHVELSWWVNGKEVHSGVS | |
| | TDPQPLKEQPALNDSRYCLSSRLRVSATFWQNPRNHFRCQVQFYGLSENDEWTQDRAKPVTQI | |
| | VSAEAWGRADCGFTSESYQQGVLSATILYEILLGKATLYAVLVSALVLMAMVKRKDSRG | |
| 130 | ATGGGTACCAGCCTCCTCTGCTGGATGGCCCTGTGTCTCCTGGGGGCAGATCACGCAGATACT | TCR I Vβ (nt) |
| | GGAGTCTCCCAGGACCCCAGACACAAGATCACAAAGAGGGGACAGAATGTAACTTTCAGGTGT | |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTCCTAGCGGGAGAGACCCAGTACTTCGGGCCAGGC | |
| | ACGCGGCTCCTGGTGCTCGAG | |
| 131 | ATGGGTACCAGCCTCCTCTGCTGGATGGCCCTGTGTCTCCTGGGGGCAGATCACGCAGATACT | TCR I Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTCCTAGCGGGAGAGACCCAGTACTTCGGGCCAGGC | |
| | ACGCGGCTCCTGGTGCTCGAGGACCTGAAAAACGTGTTCCCACCCGAGGTCGCTGTGTTTGAG | |
| | CCATCAGAAGCAGAGATCTCCCACACCCAAAAGGCCACACTGGTATGCCTGGCCACAGGCTTC | |
| | TACCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCTGTCACCCAGATC | |
| | GTCAGCGCCGAGGCCTGGGGTAGAGCAGACTGTGGCTTCACCTCCGAGTCTTACCAGCAAGGG | |
| | GTCCTGTCTGCCACCATCCTCTATGAGATCTTGCTAGGGAAGGCCACCTTGTATGCCGTGCTG | |
| | GTCAGTGCCCTCGTGCTGATGGCCATGGTCAAGAGAAAGGATTCCAGAGGCTAG | |
| 132 | ATGGAGACCCTCTTGGGCCTGCTTATCCTTTGGCTGCAGCTGCAATGGGTGAGCAGCAAACAG | TCR I full |
| | GAGGTGACGCAGATTCCTGCAGCTCTGAGTGTCCCAGAAGGAGAAAACTTGGTTCTCAACTGC | construct nt |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTTCGTGGGACTACCTCAGGAACCTACAAATACATCTTTGGAACAGGC | |
| | ACCAGGCTGAAGGTTTTAGCAAATATCCAGAACCCTGACCCTGCCGTGTACCAGCTGAGAGAC | |
| | TCTAAATCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGTCTATGGAC | |
| | TTCAAGAGCAACAGTGCTGTGGCCTGGAGCAACAAATCTGACTTTGCATGTGCAAACGCCTTC | |
| | AACAACAGCATTATTCCAGAAGACACCTTCTTCCCCAGCCCAGAAAGTTCCTGTGATGTCAAG | |
| | CTGGTCGAGAAAAGCTTTGAAACAGATACGAACCTAAACTTTCAAAACCTGTCAGTGATTGGG | |
| | TTCCGAATCCTCCTCCTGAAAGTGGCCGGGTTTAATCTGCTCATGACGCTGCGGCTGTGGTCC | |
| | AGCGGCTCCGGAGCCACGAACTTCTCTCTGTTAAAGCAAGCAGGAGACGTGGAAGAAAACCCC | |
| | GGTCCCATGGGTACCAGCCTCCTCTGCTGGATGGCCCTGTGTCTCCTGGGGGCAGATCACGCA | |
| | GATACTGGAGTCTCCCAGGACCCCAGACACAAGATCACAAAGAGGGGACAGAATGTAACTTTC | |
| | AGGTGTGATCCAATTTCTGAACACAACCGCCTTTATTGGTACCGACAGACCCTGGGGCAGGGC | |
| | CCAGAGTTTCTGACTTACTTCCAGAATGAAGCTCAACTAGAAAAATCAAGGCTGCTCAGTGAT | |
| | CGGTTCTCTGCAGAGAGGCCTAAGGGATCTTTCTCCACCTTGGAGATCCAGCGCACAGAGCAG | |
| | GGGGACTCGGCCATGTATCTCTGTGCCAGCAGCTTCCTAGCGGGAGAGACCCAGTACTTCGGG | |
| | CCAGGCACGCGGCTCCTGGTGCTCGAGGACCTGAAAAACGTGTTCCCACCCGAGGTCGCTGTG | |
| | TTTGAGCCATCAGAAGCAGAGATCTCCCACACCCAAAAGGCCACACTGGTATGCCTGGCCACA | |
| | GGCTTCTACCCCGACCACGTGGAGCTGAGCTGGTGGGTGAATGGGAAGGAGGTGCACAGTGGG | |
| | GTCAGCACAGACCCGCAGCCCCTCAAGGAGCAGCCCGCCCTCAATGACTCCAGATACTGCCTG | |
| | AGCAGCCGCCTGAGGGTCTCGGCCACCTTCTGGCAGAACCCCCGCAACCACTTCCGCTGTCAA | |
| | GTCCAGTTCTACGGGCTCTCGGAGAATGACGAGTGGACCCAGGATAGGGCCAAACCTGTCACC | |
| | CAGATCGTCAGCGCCGAGGCCTGGGGTAGAGCAGACTGTGGCTTCACCTCCGAGTCTTACCAG | |
| | CAAGGGGTCCTGTCTGCCACCATCCTCTATGAGATCTTGCTAGGGAAGGCCACCTTGTATGCC | |
| | GTGCTGGTCAGTGCCCTCGTGCTGATGGCCATGGTCAAGAGAAAGGATTCCAGAGGCTAG | |
| 133 | METLLGLLILWLQLQWVSSKQEVTQIPAALSVPEGENLVLNCSFTDSAIYNLQWFRQDPGKGL | TCR I full |
| | TSLLLIQSSQREQTSGRLNASLDKSSGRSTLYIAASQPGDSATYLCAVRGTTSGTYKYIFGTG | construct aa |
| | TRLKVLANIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSSGSGATNFSLLKQAGDVEENPGPMGTSLLCWMALCLLGADHA | |
| | DTGVSQDPRHKITKRGQNVTFRCDPISEHNRLYWYRQTLGQGPEFLTYFQNEAQLEKSRLLSD | |
| | RFSAERPKGSFSTLEIQRTEQGDSAMYLCASSFLAGETQYFGPGTRLLVLEDLKNVFPPEVAV | |
| | FEPSEAEISHTQKATLVCLATGFYPDHVELSWWVNGKEVHSGVSTDPQPLKEQPALNDSRYCL | |
| | SSRLRVSATFWQNPRNHFRCQVQFYGLSENDEWTQDRAKPVTQIVSAEAWGRADCGFTSESYQ | |
| | QGVLSATILYEILLGKATLYAVLVSALVLMAMVKRKDSRG | |
| 134 | NSAFQY | TCR J Vα |
| | | CDR-1 |
| 135 | TYSSGN | TCR J Vα |
| | | CDR-2 |
| 136 | AMRATSGTYKYI | TCR J Vα |
| | | CDR-3 |
| 137 | QKEVEQDPGPLSVPEGAIVSLNCTYSNSAFQYFMWYRQYSRKGPELLMYTYSSGNKEDGRFTA | TCR J Vα |
| | QVDKSSKYISLFIRDSQPSDSATYLCAMRATSGTYKYIFGTGTRLKVLAN | (aa) |
| 138 | MKSLRVLLVILWLQLSWVWSQQKEVEQDPGPLSVPEGAIVSLNCTYSNSAFQYFMWYRQYSRK | TCR J Vα + ss |
| | GPELLMYTYSSGNKEDGRFTAQVDKSSKYISLFIRDSQPSDSATYLCAMRATSGTYKYIFGTG | (aa) |
| | TRLKVLAN | |
| 139 | MKSLRVLLVILWLQLSWVWSQQKEVEQDPGPLSVPEGAIVSLNCTYSNSAFQYFMWYRQYSRK | TCR J Vα + |
| | GPELLMYTYSSGNKEDGRFTAQVDKSSKYISLFIRDSQPSDSATYLCAMRATSGTYKYIFGTG | Cα (aa) |
| | TRLKVLANIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSS | |
| 140 | ATGAAATCCTTGAGAGTTTTACTGGTGATCCTGTGGCTTCAGTTAAGCTGGGTTTGGAGCCAA | TCR J Vα (nt) |
| | CAGAAGGAGGTGGAGCAGGATCCTGGACCACTCAGTGTTCCAGAGGGAGCCATTGTTTCTCTC | |
| | AACTGCACTTACAGCAACAGTGCTTTTCAATACTTCATGTGGTACAGACAGTATTCCAGAAAA | |
| | GGCCCTGAGTTGCTGATGTACACATACTCCAGTGGTAACAAAGAAGATGGAAGGTTTACAGCA | |
| | CAGGTCGATAAATCCAGCAAGTATATCTCCTTGTTCATCAGAGACTCACAGCCCAGTGATTCA | |
| | GCCACCTACCTCTGTGCAATGAGGGCTACCTCAGGAACCTACAAATACATCTTTGGAACAGGC | |
| | ACCAGGCTGAAGGTTTTAGCAAAT | |
| 141 | ATGAAATCCTTGAGAGTTTTACTGGTGATCCTGTGGCTTCAGTTAAGCTGGGTTTGGAGCCAA | TCR J Vα + |
| | CAGAAGGAGGTGGAGCAGGATCCTGGACCACTCAGTGTTCCAGAGGGAGCCATTGTTTCTCTC | Cα (nt) |
| | AACTGCACTTACAGCAACAGTGCTTTTCAATACTTCATGTGGTACAGACAGTATTCCAGAAAA | |
| | GGCCCTGAGTTGCTGATGTACACATACTCCAGTGGTAACAAAGAAGATGGAAGGTTTACAGCA | |
| | CAGGTCGATAAATCCAGCAAGTATATCTCCTTGTTCATCAGAGACTCACAGCCCAGTGATTCA | |
| | GCCACCTACCTCTGTGCAATGAGGGCTACCTCAGGAACCTACAAATACATCTTTGGAACAGGC | |
| | ACCAGGCTGAAGGTTTTAGCAAATATCCAGAACCCTGACCCTGCCGTGTACCAGCTGAGAGAC | |
| | TCTAAATCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGTCTATGGAC | |
| | TTCAAGAGCAACAGTGCTGTGGCCTGGAGCAACAAATCTGACTTTGCATGTGCAAACGCCTTC | |
| | AACAACAGCATTATTCCAGAAGACACCTTCTTCCCCAGCCCAGAAAGTTCCTGTGATGTCAAG | |
| | CTGGTCGAGAAAAGCTTTGAAACAGATACGAACCTAAACTTTCAAAACCTGTCAGTGATTGGG | |
| | TTCCGAATCCTCCTCCTGAAAGTGGCCGGGTTTAATCTGCTCATGACGCTGCGGCTGTGGTCC | |
| | AGC | |
| 142 | AVPGGSSYNEQF | TCR J Vβ |
| | | CDR-3 |
| 143 | DTGVSQDPRHKITKRGQNVTFRCDPISEHNRLYWYRQTLGQGPEFLTYFQNEAQLEKSRLLSD | TCR J Vβ |
| | RFSAERPKGSFSTLEIQRTEQGDSAMYLCAVPGGSSYNEQFFGPGTRLTVLE | (aa) |
| 144 | MGTSLLCWMALCLLGADHADTGVSQDPRHKITKRGQNVTFRCDPISEHNRLYWYRQTLGQGPE | TCR J Vβ + ss |
| | FLTYFQNEAQLEKSRLLSDRFSAERPKGSFSTLEIQRTEQGDSAMYLCAVPGGSSYNEQFFGP | (aa) |
| | GTRLTVLE | |
| 145 | MGTSLLCWMALCLLGADHADTGVSQDPRHKITKRGQNVTFRCDPISEHNRLYWYRQTLGQGPE | TCR J Vβ + |
| | FLTYFQNEAQLEKSRLLSDRFSAERPKGSFSTLEIQRTEQGDSAMYLCAVPGGSSYNEQFFGP | Cβ (aa) |
| | GTRLTVLEDLKNVFPPEVAVFEPSEAEISHTQKATLVCLATGFYPDHVELSWWVNGKEVHSGV | |
| | STDPQPLKEQPALNDSRYCLSSRLRVSATFWQNPRNHERCQVQFYGLSENDEWTQDRAKPVTQ | |
| | IVSAEAWGRADCGFTSESYQQGVLSATILYEILLGKATLYAVLVSALVLMAMVKRKDSRG | |
| 146 | ATGGGTACCAGCCTCCTCTGCTGGATGGCCCTGTGTCTCCTGGGGGCAGATCACGCAGATACT | TCR J Vβ (nt) |
| | GGAGTCTCCCAGGACCCCAGACACAAGATCACAAAGAGGGGACAGAATGTAACTTTCAGGTGT | |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCGTCCCTGGGGGGAGCTCCTACAATGAGCAGTTCTTCGGGCCA | |
| | GGGACACGGCTCACCGTGCTAGAG | |
| 147 | ATGGGTACCAGCCTCCTCTGCTGGATGGCCCTGTGTCTCCTGGGGGCAGATCACGCAGATACT | TCR J Vβ + |
| | GGAGTCTCCCAGGACCCCAGACACAAGATCACAAAGAGGGGACAGAATGTAACTTTCAGGTGT | Cß (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCGTCCCTGGGGGGAGCTCCTACAATGAGCAGTTCTTCGGGCCA | |
| | GGGACACGGCTCACCGTGCTAGAGGACCTGAAAAACGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 148 | ATGAAATCCTTGAGAGTTTTACTGGTGATCCTGTGGCTTCAGTTAAGCTGGGTTTGGAGCCAA | TCR J full |
| | CAGAAGGAGGTGGAGCAGGATCCTGGACCACTCAGTGTTCCAGAGGGAGCCATTGTTTCTCTC | construct nt |
| | AACTGCACTTACAGCAACAGTGCTTTTCAATACTTCATGTGGTACAGACAGTATTCCAGAAAA | |
| | GGCCCTGAGTTGCTGATGTACACATACTCCAGTGGTAACAAAGAAGATGGAAGGTTTACAGCA | |
| | CAGGTCGATAAATCCAGCAAGTATATCTCCTTGTTCATCAGAGACTCACAGCCCAGTGATTCA | |
| | GCCACCTACCTCTGTGCAATGAGGGCTACCTCAGGAACCTACAAATACATCTTTGGAACAGGC | |
| | ACCAGGCTGAAGGTTTTAGCAAATATCCAGAACCCTGACCCTGCCGTGTACCAGCTGAGAGAC | |
| | TCTAAATCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGTCTATGGAC | |
| | TTCAAGAGCAACAGTGCTGTGGCCTGGAGCAACAAATCTGACTTTGCATGTGCAAACGCCTTC | |
| | AACAACAGCATTATTCCAGAAGACACCTTCTTCCCCAGCCCAGAAAGTTCCTGTGATGTCAAG | |
| | CTGGTCGAGAAAAGCTTTGAAACAGATACGAACCTAAACTTTCAAAACCTGTCAGTGATTGGG | |
| | TTCCGAATCCTCCTCCTGAAAGTGGCCGGGTTTAATCTGCTCATGACGCTGCGGCTGTGGTCC | |
| | AGCGGCTCCGGAGCCACGAACTTCTCTCTGTTAAAGCAAGCAGGAGACGTGGAAGAAAACCCC | |
| | GGTCCCATGGGTACCAGCCTCCTCTGCTGGATGGCCCTGTGTCTCCTGGGGGCAGATCACGCA | |
| | GATACTGGAGTCTCCCAGGACCCCAGACACAAGATCACAAAGAGGGGACAGAATGTAACTTTC | |
| | AGGTGTGATCCAATTTCTGAACACAACCGCCTTTATTGGTACCGACAGACCCTGGGGCAGGGC | |
| | CCAGAGTTTCTGACTTACTTCCAGAATGAAGCTCAACTAGAAAAATCAAGGCTGCTCAGTGAT | |
| | CGGTTCTCTGCAGAGAGGCCTAAGGGATCTTTCTCCACCTTGGAGATCCAGCGCACAGAGCAG | |
| | GGGGACTCGGCCATGTATCTCTGTGCCGTCCCTGGGGGGAGCTCCTACAATGAGCAGTTCTTC | |
| | GGGCCAGGGACACGGCTCACCGTGCTAGAGGACCTGAAAAACGTGTTCCCACCCGAGGTCGCT | |
| | GTGTTTGAGCCATCAGAAGCAGAGATCTCCCACACCCAAAAGGCCACACTGGTATGCCTGGCC | |
| | ACAGGCTTCTACCCCGACCACGTGGAGCTGAGCTGGTGGGTGAATGGGAAGGAGGTGCACAGT | |
| | GGGGTCAGCACAGACCCGCAGCCCCTCAAGGAGCAGCCCGCCCTCAATGACTCCAGATACTGC | |
| | CTGAGCAGCCGCCTGAGGGTCTCGGCCACCTTCTGGCAGAACCCCCGCAACCACTTCCGCTGT | |
| | CAAGTCCAGTTCTACGGGCTCTCGGAGAATGACGAGTGGACCCAGGATAGGGCCAAACCTGTC | |
| | ACCCAGATCGTCAGCGCCGAGGCCTGGGGTAGAGCAGACTGTGGCTTCACCTCCGAGTCTTAC | |
| | CAGCAAGGGGTCCTGTCTGCCACCATCCTCTATGAGATCTTGCTAGGGAAGGCCACCTTGTAT | |
| | GCCGTGCTGGTCAGTGCCCTCGTGCTGATGGCCATGGTCAAGAGAAAGGATTCCAGAGGCTAG | |
| 149 | MKSLRVLLVILWLQLSWVWSQQKEVEQDPGPLSVPEGAIVSLNCTYSNSAFQYFMWYRQYSRK | TCR J full |
| | GPELLMYTYSSGNKEDGRFTAQVDKSSKYISLFIRDSQPSDSATYLCAMRATSGTYKYIFGTG | construct aa |
| | TRLKVLANIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSSGSGATNFSLLKQAGDVEENPGPMGTSLLCWMALCLLGADHA | |
| | DTGVSQDPRHKITKRGQNVTFRCDPISEHNRLYWYRQTLGQGPEFLTYFQNEAQLEKSRLLSD | |
| | RFSAERPKGSFSTLEIQRTEQGDSAMYLCAVPGGSSYNEQFFGPGTRLTVLEDLKNVFPPEVA | |
| | VFEPSEAEISHTQKATLVCLATGFYPDHVELSWWVNGKEVHSGVSTDPQPLKEQPALNDSRYC | |
| | LSSRLRVSATFWQNPRNHFRCQVQFYGLSENDEWTQDRAKPVTQIVSAEAWGRADCGFTSESY | |
| | QQGVLSATILYEILLGKATLYAVLVSALVLMAMMKRKDSRG | |
| 150 | TISGTDY | TCR K Vα |
| | | CDR-1 |
| 151 | GLTSN | TCR K Vα |
| | | CDR-2 |
| 152 | ILRAGSGTYKYI | TCR K Vα |
| | | CDR-3 |
| 153 | DAKTTQPNSMESNEEEPVHLPCNHSTISGTDYIHWYRQLPSQGPEYVIHGLTSNVNNRMASLA | TCR K Vα |
| | IAEDRKSSTLILHRATLRDAAVYYCILRAGSGTYKYIFGTGTRLKVLAN | (aa) |
| 154 | MKLVTSITVLLSLGIMGDAKTTQPNSMESNEEEPVHLPCNHSTISGTDYIHWYRQLPSQGPEY | TCR K Vα + |
| | VIHGLTSNVNNRMASLAIAEDRKSSTLILHRATLRDAAVYYCILRAGSGTYKYIFGTGTRLKV | ss (aa) |
| | LAN | |
| 155 | MKLVTSITVLLSLGIMGDAKTTQPNSMESNEEEPVHLPCNHSTISGTDYIHWYRQLPSQGPEY | TCR K Vα + |
| | VIHGLTSNVNNRMASLAIAEDRKSSTLILHRATLRDAAVYYCILRAGSGTYKYIFGTGTRLKV | Cα (aa) |
| | LANIQNPDPAVYQLRDSKSSDKSVCLFTDFDSQTNVSQSKDSDVYITDKTVLDMRSMDFKSNS | |
| | AVAWSNKSDFACANAFNNSIIPEDTFFPSPESSCDVKLVEKSFETDTNLNFQNLSVIGFRILL | |
| | LKVAGFNLLMTLRLWSS | |
| 156 | ATGAAGTTGGTGACAAGCATTACTGTACTCCTATCTTTGGGTATTATGGGTGATGCTAAGACC | TCR K Vα |
| | ACACAGCCAAATTCAATGGAGAGTAACGAAGAAGAGCCTGTTCACTTGCCTTGTAACCACTCC | (nt) |
| | ACAATCAGTGGAACTGATTACATACATTGGTATCGACAGCTTCCCTCCCAGGGTCCAGAGTAC | |
| | GTGATTCATGGTCTTACAAGCAATGTGAACAACAGAATGGCCTCTCTGGCAATCGCTGAAGAC | |
| | AGAAAGTCCAGTACCTTGATCCTGCACCGTGCTACCTTGAGAGATGCTGCTGTGTACTACTGC | |
| | ATCCTGAGAGCGGGCTCAGGAACCTACAAATACATCTTTGGAACAGGCACCAGGCTGAAGGTT | |
| | CTAGCAAAT | |
| 157 | ATGAAGTTGGTGACAAGCATTACTGTACTCCTATCTTTGGGTATTATGGGTGATGCTAAGACC | TCR K Vα + |
| | ACACAGCCAAATTCAATGGAGAGTAACGAAGAAGAGCCTGTTCACTTGCCTTGTAACCACTCC | Cα (nt) |
| | ACAATCAGTGGAACTGATTACATACATTGGTATCGACAGCTTCCCTCCCAGGGTCCAGAGTAC | |
| | GTGATTCATGGTCTTACAAGCAATGTGAACAACAGAATGGCCTCTCTGGCAATCGCTGAAGAC | |
| | AGAAAGTCCAGTACCTTGATCCTGCACCGTGCTACCTTGAGAGATGCTGCTGTGTACTACTGC | |
| | ATCCTGAGAGCGGGCTCAGGAACCTACAAATACATCTTTGGAACAGGCACCAGGCTGAAGGTT | |
| | CTAGCAAATATCCAGAACCCTGACCCTGCCGTGTACCAGCTGAGAGACTCTAAATCCAGTGAC | |
| | AAGTCTGTCTGCCTATTCACCGATTTTGATTCTCAAACAAATGTGTCACAAAGTAAGGATTCT | |
| | GATGTGTATATCACAGACAAAACTGTGCTAGACATGAGGTCTATGGACTTCAAGAGCAACAGT | |
| | GCTGTGGCCTGGAGCAACAAATCTGACTTTGCATGTGCAAACGCCTTCAACAACAGCATTATT | |
| | CCAGAAGACACCTTCTTCCCCAGCCCAGAAAGTTCCTGTGATGTCAAGCTGGTCGAGAAAAGC | |
| | TTTGAAACAGATACGAACCTAAACTTTCAAAACCTGTCAGTGATTGGGTTCCGAATCCTCCTC | |
| | CTGAAAGTGGCCGGGTTTAATCTGCTCATGACGCTGCGGCTGTGGTCCAGC | |
| 158 | ASSPGTVYNEQF | TCR K, S Vβ |
| | | CDR-3 |
| 159 | DTGVSQDPRHKITKRGQNVTFRCDPISEHNRLYWYRQTLGQGPEFLTYFQNEAQLEKSRLLSD | TCR K, S Vβ |
| | RFSAERPKGSFSTLEIQRTEQGDSAMYLCASSPGTVYNEQFFGPGTRLTVLE | (aa) |
| 160 | MGTSLLCWMALCLLGADHADTGVSQDPRHKITKRGQNVTFRCDPISEHNRLYWYRQTLGQGPE | TCR K, S Vβ + |
| | FLTYFQNEAQLEKSRLLSDRFSAERPKGSFSTLEIQRTEQGDSAMYLCASSPGTVYNEQFFGP | ss (aa) |
| | GTRLTVLE | |
| 161 | MGTSLLCWMALCLLGADHADTGVSQDPRHKITKRGQNVTFRCDPISEHNRLYWYRQTLGQGPE | TCR K, S Vβ + |
| | FLTYFQNEAQLEKSRLLSDRFSAERPKGSFSTLEIQRTEQGDSAMYLCASSPGTVYNEQFFGP | Cβ (aa) |
| | GTRLTVLEDLKNVFPPEVAVFEPSEAEISHTQKATLVCLATGFYPDHVELSWWVNGKEVHSGV | |
| | STDPQPLKEQPALNDSRYCLSSRLRVSATFWQNPRNHFRCQVQFYGLSENDEWTQDRAKPVTQ | |
| | IVSAEAWGRADCGFTSESYQQGVLSATILYEILLGKATLYAVLVSALVLMAMVKRKDSRG | |
| 162 | ATGGGTACCAGCCTCCTCTGCTGGATGGCCCTGTGTCTCCTGGGGGCAGATCACGCAGATACT | TCR K Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCCCCGGGACAGTTTACAATGAGCAGTTCTTCGGGCCA | |
| | GGGACACGGCTCACCGTGCTAGAG | |
| 163 | ATGGGTACCAGCCTCCTCTGCTGGATGGCCCTGTGTCTCCTGGGGGCAGATCACGCAGATACT | TCR K Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCCCCGGGACAGTTTACAATGAGCAGTTCTTCGGGCCA | |
| | GGGACACGGCTCACCGTGCTAGAGGACCTGAAAAACGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 164 | ATGAAGTTGGTGACAAGCATTACTGTACTCCTATCTTTGGGTATTATGGGTGATGCTAAGACC | TCR K full |
| | ACACAGCCAAATTCAATGGAGAGTAACGAAGAAGAGCCTGTTCACTTGCCTTGTAACCACTCC | construct nt |
| | ACAATCAGTGGAACTGATTACATACATTGGTATCGACAGCTTCCCTCCCAGGGTCCAGAGTAC | |
| | GTGATTCATGGTCTTACAAGCAATGTGAACAACAGAATGGCCTCTCTGGCAATCGCTGAAGAC | |
| | AGAAAGTCCAGTACCTTGATCCTGCACCGTGCTACCTTGAGAGATGCTGCTGTGTACTACTGC | |
| | ATCCTGAGAGCGGGCTCAGGAACCTACAAATACATCTTTGGAACAGGCACCAGGCTGAAGGTT | |
| | CTAGCAAATATCCAGAACCCTGACCCTGCCGTGTACCAGCTGAGAGACTCTAAATCCAGTGAC | |
| | AAGTCTGTCTGCCTATTCACCGATTTTGATTCTCAAACAAATGTGTCACAAAGTAAGGATTCT | |
| | GATGTGTATATCACAGACAAAACTGTGCTAGACATGAGGTCTATGGACTTCAAGAGCAACAGT | |
| | GCTGTGGCCTGGAGCAACAAATCTGACTTTGCATGTGCAAACGCCTTCAACAACAGCATTATT | |
| | CCAGAAGACACCTTCTTCCCCAGCCCAGAAAGTTCCTGTGATGTCAAGCTGGTCGAGAAAAGC | |
| | TTTGAAACAGATACGAACCTAAACTTTCAAAACCTGTCAGTGATTGGGTTCCGAATCCTCCTC | |
| | CTGAAAGTGGCCGGGTTTAATCTGCTCATGACGCTGCGGCTGTGGTCCAGCGGCTCCGGAGCC | |
| | ACGAACTTCTCTCTGTTAAAGCAAGCAGGAGACGTGGAAGAAAACCCCGGTCCCATGGGTACC | |
| | AGCCTCCTCTGCTGGATGGCCCTGTGTCTCCTGGGGGCAGATCACGCAGATACTGGAGTCTCC | |
| | CAGGACCCCAGACACAAGATCACAAAGAGGGGACAGAATGTAACTTTCAGGTGTGATCCAATT | |
| | TCTGAACACAACCGCCTTTATTGGTACCGACAGACCCTGGGGCAGGGCCCAGAGTTTCTGACT | |
| | TACTTCCAGAATGAAGCTCAACTAGAAAAATCAAGGCTGCTCAGTGATCGGTTCTCTGCAGAG | |
| | AGGCCTAAGGGATCTTTCTCCACCTTGGAGATCCAGCGCACAGAGCAGGGGGACTCGGCCATG | |
| | TATCTCTGTGCCAGCAGCCCCGGGACAGTTTACAATGAGCAGTTCTTCGGGCCAGGGACACGG | |
| | CTCACCGTGCTAGAGGACCTGAAAAACGTGTTCCCACCCGAGGTCGCTGTGTTTGAGCCATCA | |
| | GAAGCAGAGATCTCCCACACCCAAAAGGCCACACTGGTATGCCTGGCCACAGGCTTCTACCCC | |
| | GACCACGTGGAGCTGAGCTGGTGGGTGAATGGGAAGGAGGTGCACAGTGGGGTCAGCACAGAC | |
| | CCGCAGCCCCTCAAGGAGCAGCCCGCCCTCAATGACTCCAGATACTGCCTGAGCAGCCGCCTG | |
| | AGGGTCTCGGCCACCTTCTGGCAGAACCCCCGCAACCACTTCCGCTGTCAAGTCCAGTTCTAC | |
| | GGGCTCTCGGAGAATGACGAGTGGACCCAGGATAGGGCCAAACCTGTCACCCAGATCGTCAGC | |
| | GCCGAGGCCTGGGGTAGAGCAGACTGTGGCTTCACCTCCGAGTCTTACCAGCAAGGGGTCCTG | |
| | TCTGCCACCATCCTCTATGAGATCTTGCTAGGGAAGGCCACCTTGTATGCCGTGCTGGTCAGT | |
| | GCCCTCGTGCTGATGGCCATGGTCAAGAGAAAGGATTCCAGAGGCTAG | |
| 165 | MKLVTSITVLLSLGIMGDAKTTQPNSMESNEEEPVHLPCNHSTISGTDYIHWYRQLPSQGPEY | TCR K full |
| | VIHGLTSNVNNRMASLAIAEDRKSSTLILHRATLRDAAVYYCILRAGSGTYKYIFGTGTRLKV | construct aa |
| | LANIQNPDPAVYQLRDSKSSDKSVCLFTDFDSQTNVSQSKDSDVYITDKTVLDMRSMDFKSNS | |
| | AVAWSNKSDFACANAFNNSIIPEDTFFPSPESSCDVKLVEKSFETDTNLNFQNLSVIGFRILL | |
| | LKVAGFNLLMTLRLWSSGSGATNFSLLKQAGDVEENPGPMGTSLLCWMALCLLGADHADTGVS | |
| | QDPRHKITKRGQNVTFRCDPISEHNRLYWYRQTLGQGPEFLTYFQNEAQLEKSRLLSDRFSAE | |
| | RPKGSFSTLEIQRTEQGDSAMYLCASSPGTVYNEQFFGPGTRLTVLEDLKNVEPPEVAVFEPS | |
| | EAEISHTQKATLVCLATGFYPDHVELSWWVNGKEVHSGVSTDPQPLKEQPALNDSRYCLSSRL | |
| | RVSATFWQNPRNHFRCQVQFYGLSENDEWTQDRAKPVTQIVSAEAWGRADCGFTSESYQQGVL | |
| | SATILYEILLGKATLYAVLVSALVLMAMVKRKDSRG | |
| 166 | AVTRTGANNLF | TCR L Vα |
| | | CDR-3 |
| 167 | KQEVTQIPAALSVPEGENLVLNCSFTDSAIYNLQWFRQDPGKGLTSLLLIQSSQREQTSGRLN | TCR L Vα |
| | ASLDKSSGRSTLYIAASQPGDSATYLCAVTRTGANNLFFGTGTRLTVIPY | (aa) |
| 168 | METLLGLLILWLQLQWVSSKQEVTQIPAALSVPEGENLVLNCSFTDSAIYNLQWFRQDPGKGL | TCR L Vα + |
| | TSLLLIQSSQREQTSGRLNASLDKSSGRSTLYIAASQPGDSATYLCAVTRTGANNLFFGTGTR | ss (aa) |
| | LTVIPY | |
| 169 | METLLGLLILWLQLQWVSSKQEVTQIPAALSVPEGENLVLNCSFTDSAIYNLQWFRQDPGKGL | TCR L Vα + |
| | TSLLLIQSSQREQTSGRLNASLDKSSGRSTLYIAASQPGDSATYLCAVTRTGANNLFFGTGTR | Cα (aa) |
| | LTVIPYIQNPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGFR | |
| | ILLLKVAGFNLLMTLRLWSS | |
| 170 | ATGGAGACCCTCTTGGGCCTGCTTATCCTTTGGCTGCAGCTGCAATGGGTGAGCAGCAAACAG | TCR L Vα |
| | GAGGTGACGCAGATTCCTGCAGCTCTGAGTGTCCCAGAAGGAGAAAACTTGGTTCTCAACTGC | (nt) |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTGACGAGGACTGGGGCAAACAACCTCTTCTTTGGGACTGGAACGAGA | |
| | CTCACCGTTATTCCCTAT | |
| 171 | ATGGAGACCCTCTTGGGCCTGCTTATCCTTTGGCTGCAGCTGCAATGGGTGAGCAGCAAACAG | TCR L Vα + |
| | GAGGTGACGCAGATTCCTGCAGCTCTGAGTGTCCCAGAAGGAGAAAACTTGGTTCTCAACTGC | Cα (nt) |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTGACGAGGACTGGGGCAAACAACCTCTTCTTTGGGACTGGAACGAGA | |
| | CTCACCGTTATTCCCTATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATGTGTCACAAAGT | |
| | AAGGATTCTGATGTGTATATCACAGACAAAACTGTGCTAGACATGAGGTCTATGGACTTCAAG | |
| | AGCAACAGTGCTGTGGCCTGGAGCAACAAATCTGACTTTGCATGTGCAAACGCCTTCAACAAC | |
| | AGCATTATTCCAGAAGACACCTTCTTCCCCAGCCCAGAAAGTTCCTGTGATGTCAAGCTGGTC | |
| | GAGAAAAGCTTTGAAACAGATACGAACCTAAACTTTCAAAACCTGTCAGTGATTGGGTTCCGA | |
| | ATCCTCCTCCTGAAAGTGGCCGGGTTTAATCTGCTCATGACGCTGCGGCTGTGGTCCAGC | |
| 172 | ASSLGVLGIGYT | TCR L Vβ |
| | | CDR-3 |
| 173 | DTGVSQDPRHKITKRGQNVTFRCDPISEHNRLYWYRQTLGQGPEFLTYFQNEAQLEKSRLLSD | TCR L Vβ |
| | RFSAERPKGSFSTLEIQRTEQGDSAMYLCASSLGVLGIGYTFGSGTRLTVVE | (aa) |
| 174 | MGTSLLCWMALCLLGADHADTGVSQDPRHKITKRGQNVTFRCDPISEHNRLYWYRQTLGQGPE | TCR L Vβ + |
| | FLTYFQNEAQLEKSRLLSDRFSAERPKGSFSTLEIQRTEQGDSAMYLCASSLGVLGIGYTFGS | ss (aa) |
| | GTRLTVVE | |
| 175 | MGTSLLCWMALCLLGADHADTGVSQDPRHKITKRGQNVTFRCDPISEHNRLYWYRQTLGQGPE | TCR L Vβ + |
| | FLTYFQNEAQLEKSRLLSDRFSAERPKGSFSTLEIQRTEQGDSAMYLCASSLGVLGIGYTFGS | Cβ (aa) |
| | GTRLTVVEDLNKVFPPEVAVFEPSEAEISHTQKATLVCLATGFFPDHVELSWWVNGKEVHSGV | |
| | STDPQPLKEQPALNDSRYCLSSRLRVSATFWQNPRNHERCQVQFYGLSENDEWTQDRAKPVTQ | |
| | IVSAEAWGRADCGFTSVSYQQGVLSATILYEILLGKATLYAVLVSALVLMAMVKRKDF | |
| 176 | ATGGGTACCAGCCTCCTCTGCTGGATGGCCCTGTGTCTCCTGGGGGCAGATCACGCAGATACT | TCR L Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGGAGTACTCGGGATCGGCTACACCTTCGGTTCG | |
| | GGGACCAGGTTAACCGTTGTAGAG | |
| 177 | ATGGGTACCAGCCTCCTCTGCTGGATGGCCCTGTGTCTCCTGGGGGCAGATCACGCAGATACT | TCR L Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGGAGTACTCGGGATCGGCTACACCTTCGGTTCG | |
| | GGGACCAGGTTAACCGTTGTAGAGGACCTGAACAAGGTGTTCCCACCCGAGGTCGCTGTGTTT | |
| | GAGCCATCAGAAGCAGAGATCTCCCACACCCAAAAGGCCACACTGGTGTGCCTGGCCACAGGC | |
| | TTCTTCCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCCGTCACCCAG | |
| | ATCGTCAGCGCCGAGGCCTGGGGTAGAGCAGACTGTGGCTTTACCTCGGTGTCCTACCAGCAA | |
| | GGGGTCCTGTCTGCCACCATCCTCTATGAGATCCTGCTAGGGAAGGCCACCCTGTATGCTGTG | |
| | CTGGTCAGCGCCCTTGTGTTGATGGCCATGGTCAAGAGAAAGGATTTCTGA | |
| 178 | ATGGAGACCCTCTTGGGCCTGCTTATCCTTTGGCTGCAGCTGCAATGGGTGAGCAGCAAACAG | TCR L full |
| | GAGGTGACGCAGATTCCTGCAGCTCTGAGTGTCCCAGAAGGAGAAAACTTGGTTCTCAACTGC | construct nt |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTGACGAGGACTGGGGCAAACAACCTCTTCTTTGGGACTGGAACGAGA | |
| | CTCACCGTTATTCCCTATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATGTGTCACAAAGT | |
| | AAGGATTCTGATGTGTATATCACAGACAAAACTGTGCTAGACATGAGGTCTATGGACTTCAAG | |
| | AGCAACAGTGCTGTGGCCTGGAGCAACAAATCTGACTTTGCATGTGCAAACGCCTTCAACAAC | |
| | AGCATTATTCCAGAAGACACCTTCTTCCCCAGCCCAGAAAGTTCCTGTGATGTCAAGCTGGTC | |
| | GAGAAAAGCTTTGAAACAGATACGAACCTAAACTTTCAAAACCTGTCAGTGATTGGGTTCCGA | |
| | ATCCTCCTCCTGAAAGTGGCCGGGTTTAATCTGCTCATGACGCTGCGGCTGTGGTCCAGCGGC | |
| | TCCGGAGCCACGAACTTCTCTCTGTTAAAGCAAGCAGGAGACGTGGAAGAAAACCCCGGTCCC | |
| | ATGGGTACCAGCCTCCTCTGCTGGATGGCCCTGTGTCTCCTGGGGGCAGATCACGCAGATACT | |
| | GGAGTCTCCCAGGACCCCAGACACAAGATCACAAAGAGGGGACAGAATGTAACTTTCAGGTGT | |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGGAGTACTCGGGATCGGCTACACCTTCGGTTCG | |
| | GGGACCAGGTTAACCGTTGTAGAGGACCTGAACAAGGTGTTCCCACCCGAGGTCGCTGTGTTT | |
| | GAGCCATCAGAAGCAGAGATCTCCCACACCCAAAAGGCCACACTGGTGTGCCTGGCCACAGGC | |
| | TTCTTCCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCCGTCACCCAG | |
| | ATCGTCAGCGCCGAGGCCTGGGGTAGAGCAGACTGTGGCTTTACCTCGGTGTCCTACCAGCAA | |
| | GGGGTCCTGTCTGCCACCATCCTCTATGAGATCCTGCTAGGGAAGGCCACCCTGTATGCTGTG | |
| | CTGGTCAGCGCCCTTGTGTTGATGGCCATGGTCAAGAGAAAGGATTTCTGA | |
| 179 | METLLGLLILWLQLQWVSSKQEVTQIPAALSVPEGENLVLNCSFTDSAIYNLQWFRQDPGKGL | TCR L full |
| | TSLLLIQSSQREQTSGRLNASLDKSSGRSTLYIAASQPGDSATYLCAVTRTGANNLFFGTGTR | construct aa |
| | LTVIPYIQNPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGER | |
| | ILLLKVAGFNLLMTLRLWSSGSGATNFSLLKQAGDVEENPGPMGTSLLCWMALCLLGADHADT | |
| | GVSQDPRHKITKRGQNVTFRCDPISEHNRLYWYRQTLGQGPEFLTYFQNEAQLEKSRLLSDRF | |
| | SAERPKGSFSTLEIQRTEQGDSAMYLCASSLGVLGIGYTFGSGTRLTVVEDLNKVFPPEVAVF | |
| | EPSEAEISHTQKATLVCLATGFFPDHVELSWWVNGKEVHSGVSTDPQPLKEQPALNDSRYCLS | |
| | SRLRVSATFWQNPRNHFRCQVQFYGLSENDEWTQDRAKPVTQIVSAEAWGRADCGFTSVSYQQ | |
| | GVLSATILYEILLGKATLYAVLVSALVLMAMVKRKDF | |
| 180 | AVRGSTSGTYKYI | TCR M Vα |
| | | CDR-3 |
| 181 | KQEVTQIPAALSVPEGENLVLNCSFTDSAIYNLQWFRQDPGKGLTSLLLIQSSQREQTSGRLN | TCR M Vα |
| | ASLDKSSGRSTLYIAASQPGDSATYLCAVRGSTSGTYKYIFGTGTRLKVLAN | (aa) |
| 182 | METLLGLLILWLQLQWVSSKQEVTQIPAALSVPEGENLVLNCSFTDSAIYNLQWFRQDPGKGL | TCR M Vα + |
| | TSLLLIQSSQREQTSGRLNASLDKSSGRSTLYIAASQPGDSATYLCAVRGSTSGTYKYIFGTG | ss (aa) |
| | TRLKVLAN | |
| 183 | METLLGLLILWLQLQWVSSKQEVTQIPAALSVPEGENLVLNCSFTDSAIYNLQWFRQDPGKGL | TCR M Vα + |
| | TSLLLIQSSQREQTSGRLNASLDKSSGRSTLYIAASQPGDSATYLCAVRGSTSGTYKYIFGTG | Cα (aa) |
| | TRLKVLANIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSS | |
| 184 | ATGGAGACCCTCTTGGGCCTGCTTATCCTTTGGCTGCAGCTGCAATGGGTGAGCAGCAAACAG | TCR M Vα |
| | GAGGTGACGCAGATTCCTGCAGCTCTGAGTGTCCCAGAAGGAGAAAACTTGGTTCTCAACTGC | (nt) |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTGAGGGGGAGTACCTCAGGAACCTACAAATACATCTTTGGAACAGGC | |
| | ACCAGGCTGAAGGTTTTAGCAAAT | |
| 185 | ATGGAGACCCTCTTGGGCCTGCTTATCCTTTGGCTGCAGCTGCAATGGGTGAGCAGCAAACAG | TCR M Vα + |
| | GAGGTGACGCAGATTCCTGCAGCTCTGAGTGTCCCAGAAGGAGAAAACTTGGTTCTCAACTGC | Cα (nt) |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTGAGGGGGAGTACCTCAGGAACCTACAAATACATCTTTGGAACAGGC | |
| | ACCAGGCTGAAGGTTTTAGCAAATATCCAGAACCCTGACCCTGCCGTGTACCAGCTGAGAGAC | |
| | TCTAAATCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGTCTATGGAC | |
| | TTCAAGAGCAACAGTGCTGTGGCCTGGAGCAACAAATCTGACTTTGCATGTGCAAACGCCTTC | |
| | AACAACAGCATTATTCCAGAAGACACCTTCTTCCCCAGCCCAGAAAGTTCCTGTGATGTCAAG | |
| | CTGGTCGAGAAAAGCTTTGAAACAGATACGAACCTAAACTTTCAAAACCTGTCAGTGATTGGG | |
| | TTCCGAATCCTCCTCCTGAAAGTGGCCGGGTTTAATCTGCTCATGACGCTGCGGCTGTGGTCC | |
| | AGC | |
| 186 | ASSLVAGETQY | TCR M Vβ |
| | | CDR-3 |
| 187 | DTGVSQDPRHKITKRGQNVTFRCDPISEHNRLYWYRQTLGQGPEFLTYFQNEAQLEKSRLLSD | TCR M Vβ |
| | RFSAERPKGSFSTLEIQRTEQGDSAMYLCASSLVAGETQYFGPGTRLLVLE | (aa) |
| 188 | MGTSLLCWMALCLLGADHADTGVSQDPRHKITKRGQNVTFRCDPISEHNRLYWYRQTLGQGPE | TCR M Vβ + |
| | FLTYFQNEAQLEKSRLLSDRFSAERPKGSFSTLEIQRTEQGDSAMYLCASSLVAGETQYFGPG | ss (aa) |
| | TRLLVLE | |
| 189 | MGTSLLCWMALCLLGADHADTGVSQDPRHKITKRGQNVTFRCDPISEHNRLYWYRQTLGQGPE | TCR M Vβ + |
| | FLTYFQNEAQLEKSRLLSDRFSAERPKGSFSTLEIQRTEQGDSAMYLCASSLVAGETQYFGPG | Cβ (aa) |
| | TRLLVLEDLKNVFPPEVAVFEPSEAEISHTQKATLVCLATGFYPDHVELSWWVNGKEVHSGVS | |
| | TDPQPLKEQPALNDSRYCLSSRLRVSATFWQNPRNHERCQVQFYGLSENDEWTQDRAKPVTQI | |
| | VSAEAWGRADCGFTSESYQQGVLSATILYEILLGKATLYAVLVSALVLMAMVKRKDSRG | |
| 190 | ATGGGTACCAGCCTCCTCTGCTGGATGGCCCTGTGTCTCCTGGGGGCAGATCACGCAGATACT | TCR M Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGTCGCCGGAGAGACCCAGTACTTCGGGCCAGGC | |
| | ACGCGGCTCCTGGTGCTCGAG | |
| 191 | ATGGGTACCAGCCTCCTCTGCTGGATGGCCCTGTGTCTCCTGGGGGCAGATCACGCAGATACT | TCR M Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGTCGCCGGAGAGACCCAGTACTTCGGGCCAGGC | |
| | ACGCGGCTCCTGGTGCTCGAGGACCTGAAAAACGTGTTCCCACCCGAGGTCGCTGTGTTTGAG | |
| | CCATCAGAAGCAGAGATCTCCCACACCCAAAAGGCCACACTGGTATGCCTGGCCACAGGCTTC | |
| | TACCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCTGTCACCCAGATC | |
| | GTCAGCGCCGAGGCCTGGGGTAGAGCAGACTGTGGCTTCACCTCCGAGTCTTACCAGCAAGGG | |
| | GTCCTGTCTGCCACCATCCTCTATGAGATCTTGCTAGGGAAGGCCACCTTGTATGCCGTGCTG | |
| | GTCAGTGCCCTCGTGCTGATGGCCATGGTCAAGAGAAAGGATTCCAGAGGCTAG | |
| 192 | ATGGAGACCCTCTTGGGCCTGCTTATCCTTTGGCTGCAGCTGCAATGGGTGAGCAGCAAACAG | TCR M full |
| | GAGGTGACGCAGATTCCTGCAGCTCTGAGTGTCCCAGAAGGAGAAAACTTGGTTCTCAACTGC | construct nt |
| | AGTTTCACTGATAGCGCTATTTACAACCTCCAGTGGTTTAGGCAGGACCCTGGGAAAGGTCTC | |
| | ACATCTCTGTTGCTTATTCAGTCAAGTCAGAGAGAGCAAACAAGTGGAAGACTTAATGCCTCG | |
| | CTGGATAAATCATCAGGACGTAGTACTTTATACATTGCAGCTTCTCAGCCTGGTGACTCAGCC | |
| | ACCTACCTCTGTGCTGTGAGGGGGAGTACCTCAGGAACCTACAAATACATCTTTGGAACAGGC | |
| | ACCAGGCTGAAGGTTTTAGCAAATATCCAGAACCCTGACCCTGCCGTGTACCAGCTGAGAGAC | |
| | TCTAAATCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGTCTATGGAC | |
| | TTCAAGAGCAACAGTGCTGTGGCCTGGAGCAACAAATCTGACTTTGCATGTGCAAACGCCTTC | |
| | AACAACAGCATTATTCCAGAAGACACCTTCTTCCCCAGCCCAGAAAGTTCCTGTGATGTCAAG | |
| | CTGGTCGAGAAAAGCTTTGAAACAGATACGAACCTAAACTTTCAAAACCTGTCAGTGATTGGG | |
| | TTCCGAATCCTCCTCCTGAAAGTGGCCGGGTTTAATCTGCTCATGACGCTGCGGCTGTGGTCC | |
| | AGCGGCTCCGGAGCCACGAACTTCTCTCTGTTAAAGCAAGCAGGAGACGTGGAAGAAAACCCC | |
| | GGTCCCATGGGTACCAGCCTCCTCTGCTGGATGGCCCTGTGTCTCCTGGGGGCAGATCACGCA | |
| | GATACTGGAGTCTCCCAGGACCCCAGACACAAGATCACAAAGAGGGGACAGAATGTAACTTTC | |
| | AGGTGTGATCCAATTTCTGAACACAACCGCCTTTATTGGTACCGACAGACCCTGGGGCAGGGC | |
| | CCAGAGTTTCTGACTTACTTCCAGAATGAAGCTCAACTAGAAAAATCAAGGCTGCTCAGTGAT | |
| | CGGTTCTCTGCAGAGAGGCCTAAGGGATCTTTCTCCACCTTGGAGATCCAGCGCACAGAGCAG | |
| | GGGGACTCGGCCATGTATCTCTGTGCCAGCAGCTTAGTCGCCGGAGAGACCCAGTACTTCGGG | |
| | CCAGGCACGCGGCTCCTGGTGCTCGAGGACCTGAAAAACGTGTTCCCACCCGAGGTCGCTGTG | |
| | TTTGAGCCATCAGAAGCAGAGATCTCCCACACCCAAAAGGCCACACTGGTATGCCTGGCCACA | |
| | GGCTTCTACCCCGACCACGTGGAGCTGAGCTGGTGGGTGAATGGGAAGGAGGTGCACAGTGGG | |
| | GTCAGCACAGACCCGCAGCCCCTCAAGGAGCAGCCCGCCCTCAATGACTCCAGATACTGCCTG | |
| | AGCAGCCGCCTGAGGGTCTCGGCCACCTTCTGGCAGAACCCCCGCAACCACTTCCGCTGTCAA | |
| | GTCCAGTTCTACGGGCTCTCGGAGAATGACGAGTGGACCCAGGATAGGGCCAAACCTGTCACC | |
| | CAGATCGTCAGCGCCGAGGCCTGGGGTAGAGCAGACTGTGGCTTCACCTCCGAGTCTTACCAG | |
| | CAAGGGGTCCTGTCTGCCACCATCCTCTATGAGATCTTGCTAGGGAAGGCCACCTTGTATGCC | |
| | GTGCTGGTCAGTGCCCTCGTGCTGATGGCCATGGTCAAGAGAAAGGATTCCAGAGGCTAG | |
| 193 | METLLGLLILWLQLQWVSSKQEVTQIPAALSVPEGENLVLNCSFTDSAIYNLQWFRQDPGKGL | TCR M full |
| | TSLLLIQSSQREQTSGRLNASLDKSSGRSTLYIAASQPGDSATYLCAVRGSTSGTYKYIFGTG | construct aa |
| | TRLKVLANIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSSGSGATNFSLLKQAGDVEENPGPMGTSLLCWMALCLLGADHA | |
| | DTGVSQDPRHKITKRGQNVTFRCDPISEHNRLYWYRQTLGQGPEFLTYFQNEAQLEKSRLLSD | |
| | RFSAERPKGSFSTLEIQRTEQGDSAMYLCASSLVAGETQYFGPGTRLLVLEDLKNVFPPEVAV | |
| | FEPSEAEISHTQKATLVCLATGFYPDHVELSWWVNGKEVHSGVSTDPQPLKEQPALNDSRYCL | |
| | SSRLRVSATFWQNPRNHFRCQVQFYGLSENDEWTQDRAKPVTQIVSAEAWGRADCGFTSESYQ | |
| | QGVLSATILYEILLGKATLYAVLVSALVLMAMVKRKDSRG | |
| 194 | AAIVGNQFY | TCR N Vα |
| | | CDR-3 |
| 195 | GENVEQHPSTLSVQEGDSAVIKCTYSDSASNYFPWYKQELGKRPQLIIDIRSNVGEKKDQRIA | TCR N Vα |
| | VTLNKTAKHFSLHITETQPEDSAVYFCAAIVGNQFYFGTGTSLTVIPN | (aa) |
| 196 | MSIRALFIFLWLQLDLVNGENVEQHPSTLSVQEGDSAVIKCTYSDSASNYFPWYKQELGKRPQ | TCR N Vα + |
| | LIIDIRSNVGEKKDQRIAVTLNKTAKHFSLHITETQPEDSAVYFCAAIVGNQFYFGTGTSLTV | ss (aa) |
| | IPN | |
| 197 | MSIRALFIFLWLQLDLVNGENVEQHPSTLSVQEGDSAVIKCTYSDSASNYFPWYKQELGKRPQ | TCR N Vα + |
| | LIIDIRSNVGEKKDQRIAVTLNKTAKHFSLHITETQPEDSAVYFCAAIVGNQFYFGTGTSLTV | Cα (aa) |
| | IPNIQKPDPAVYQLRDSKSSDKSVCLFTDFDSQTNVSQSKDSDVYITDKTVLDMRSMDFKSNS | |
| | AVAWSNKSDFACANAFNNSIIPEDTFFPSPESSCDVKLVEKSFETDTNLNFQNLSVIGFRILL | |
| | LKVAGFNLLMTLRLWSS | |
| 198 | ATGTCCATTCGAGCTTTATTTATATTCCTGTGGCTGCAGCTGGACTTGGTGAATGGAGAGAAT | TCR N Vα |
| | GTGGAGCAGCATCCTTCAACCCTGAGTGTCCAGGAGGGAGACAGCGCTGTTATCAAGTGTACT | (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCAATAGTCGGTAACCAGTTCTATTTTGGGACAGGGACAAGTTTGACGGTC | |
| | ATTCCAAAT | |
| 199 | ATGTCCATTCGAGCTTTATTTATATTCCTGTGGCTGCAGCTGGACTTGGTGAATGGAGAGAAT | TCR N Vα + |
| | GTGGAGCAGCATCCTTCAACCCTGAGTGTCCAGGAGGGAGACAGCGCTGTTATCAAGTGTACT | Cα (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCAATAGTCGGTAACCAGTTCTATTTTGGGACAGGGACAAGTTTGACGGTC | |
| | ATTCCAAATATCCAGAAGCCTGACCCTGCCGTGTACCAGCTGAGAGACTCTAAATCCAGTGAC | |
| | AAGTCTGTCTGCCTATTCACCGATTTTGATTCTCAAACAAATGTGTCACAAAGTAAGGATTCT | |
| | GATGTGTATATCACAGACAAAACTGTGCTAGACATGAGGTCTATGGACTTCAAGAGCAACAGT | |
| | GCTGTGGCCTGGAGCAACAAATCTGACTTTGCATGTGCAAACGCCTTCAACAACAGCATTATT | |
| | CCAGAAGACACCTTCTTCCCCAGCCCAGAAAGTTCCTGTGATGTCAAGCTGGTCGAGAAAAGC | |
| | TTTGAAACAGATACGAACCTAAACTTTCAAAACCTGTCAGTGATTGGGTTCCGAATCCTCCTC | |
| | CTGAAAGTGGCCGGGTTTAATCTGCTCATGACGCTGCGGCTGTGGTCCAGC | |
| 200 | ASSETTLSEQF | TCR N Vβ |
| | | CDR-3 |
| 201 | DTGVSQDPRHKITKRGQNVTFRCDPISEHNRLYWYRQTLGQGPEFLTYFQNEAQLEKSRLLSD | TCR N Vβ |
| | RFSAERPKGSFSTLEIQRTEQGDSAMYLCASSETTLSEQFFGPGTRLTVLE | (aa) |
| 202 | MGTSLLCWMALCLLGADHADTGVSQDPRHKITKRGQNVTFRCDPISEHNRLYWYRQTLGQGPE | TCR N Vβ + |
| | FLTYFQNEAQLEKSRLLSDRFSAERPKGSFSTLEIQRTEQGDSAMYLCASSETTLSEQFFGPG | ss (aa) |
| | TRLTVLE | |
| 203 | MGTSLLCWMALCLLGADHADTGVSQDPRHKITKRGQNVTFRCDPISEHNRLYWYRQTLGQGPE | TCR N Vβ + |
| | FLTYFQNEAQLEKSRLLSDRFSAERPKGSFSTLEIQRTEQGDSAMYLCASSETTLSEQFFGPG | Cβ (aa) |
| | TRLTVLEDLKNVFPPEVAVFEPSEAEISHTQKATLVCLATGFYPDHVELSWWVNGKEVHSGVS | |
| | TDPQPLKEQPALNDSRYCLSSRLRVSATFWQNPRNHFRCQVQFYGLSENDEWTQDRAKPVTQI | |
| | VSAEAWGRADCGFTSESYQQGVLSATILYEILLGKATLYAVLVSALVLMAMVKRKDSRG | |
| 204 | ATGGGTACCAGCCTCCTCTGCTGGATGGCCCTGTGTCTCCTGGGGGCAGATCACGCAGATACT | TCR N Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGTGAAACTACGCTAAGTGAGCAGTTCTTCGGGCCAGGG | |
| | ACACGGCTCACCGTGCTAGAG | |
| 205 | ATGGGTACCAGCCTCCTCTGCTGGATGGCCCTGTGTCTCCTGGGGGCAGATCACGCAGATACT | TCR N Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGTGAAACTACGCTAAGTGAGCAGTTCTTCGGGCCAGGG | |
| | ACACGGCTCACCGTGCTAGAGGACCTGAAAAACGTGTTCCCACCCGAGGTCGCTGTGTTTGAG | |
| | CCATCAGAAGCAGAGATCTCCCACACCCAAAAGGCCACACTGGTATGCCTGGCCACAGGCTTC | |
| | TACCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCTGTCACCCAGATC | |
| | GTCAGCGCCGAGGCCTGGGGTAGAGCAGACTGTGGCTTCACCTCCGAGTCTTACCAGCAAGGG | |
| | GTCCTGTCTGCCACCATCCTCTATGAGATCTTGCTAGGGAAGGCCACCTTGTATGCCGTGCTG | |
| | GTCAGTGCCCTCGTGCTGATGGCCATGGTCAAGAGAAAGGATTCCAGAGGCTAG | |
| 206 | ATGTCCATTCGAGCTTTATTTATATTCCTGTGGCTGCAGCTGGACTTGGTGAATGGAGAGAAT | TCR N full |
| | GTGGAGCAGCATCCTTCAACCCTGAGTGTCCAGGAGGGAGACAGCGCTGTTATCAAGTGTACT | construct nt |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCAATAGTCGGTAACCAGTTCTATTTTGGGACAGGGACAAGTTTGACGGTC | |
| | ATTCCAAATATCCAGAAGCCTGACCCTGCCGTGTACCAGCTGAGAGACTCTAAATCCAGTGAC | |
| | AAGTCTGTCTGCCTATTCACCGATTTTGATTCTCAAACAAATGTGTCACAAAGTAAGGATTCT | |
| | GATGTGTATATCACAGACAAAACTGTGCTAGACATGAGGTCTATGGACTTCAAGAGCAACAGT | |
| | GCTGTGGCCTGGAGCAACAAATCTGACTTTGCATGTGCAAACGCCTTCAACAACAGCATTATT | |
| | CCAGAAGACACCTTCTTCCCCAGCCCAGAAAGTTCCTGTGATGTCAAGCTGGTCGAGAAAAGC | |
| | TTTGAAACAGATACGAACCTAAACTTTCAAAACCTGTCAGTGATTGGGTTCCGAATCCTCCTC | |
| | CTGAAAGTGGCCGGGTTTAATCTGCTCATGACGCTGCGGCTGTGGTCCAGCGGCTCCGGAGCC | |
| | ACGAACTTCTCTCTGTTAAAGCAAGCAGGAGACGTGGAAGAAAACCCCGGTCCCATGGGTACC | |
| | AGCCTCCTCTGCTGGATGGCCCTGTGTCTCCTGGGGGCAGATCACGCAGATACTGGAGTCTCC | |
| | CAGGACCCCAGACACAAGATCACAAAGAGGGGACAGAATGTAACTTTCAGGTGTGATCCAATT | |
| | TCTGAACACAACCGCCTTTATTGGTACCGACAGACCCTGGGGCAGGGCCCAGAGTTTCTGACT | |
| | TACTTCCAGAATGAAGCTCAACTAGAAAAATCAAGGCTGCTCAGTGATCGGTTCTCTGCAGAG | |
| | AGGCCTAAGGGATCTTTCTCCACCTTGGAGATCCAGCGCACAGAGCAGGGGGACTCGGCCATG | |
| | TATCTCTGTGCCAGCAGTGAAACTACGCTAAGTGAGCAGTTCTTCGGGCCAGGGACACGGCTC | |
| | ACCGTGCTAGAGGACCTGAAAAACGTGTTCCCACCCGAGGTCGCTGTGTTTGAGCCATCAGAA | |
| | GCAGAGATCTCCCACACCCAAAAGGCCACACTGGTATGCCTGGCCACAGGCTTCTACCCCGAC | |
| | CACGTGGAGCTGAGCTGGTGGGTGAATGGGAAGGAGGTGCACAGTGGGGTCAGCACAGACCCG | |
| | CAGCCCCTCAAGGAGCAGCCCGCCCTCAATGACTCCAGATACTGCCTGAGCAGCCGCCTGAGG | |
| | GTCTCGGCCACCTTCTGGCAGAACCCCCGCAACCACTTCCGCTGTCAAGTCCAGTTCTACGGG | |
| | CTCTCGGAGAATGACGAGTGGACCCAGGATAGGGCCAAACCTGTCACCCAGATCGTCAGCGCC | |
| | GAGGCCTGGGGTAGAGCAGACTGTGGCTTCACCTCCGAGTCTTACCAGCAAGGGGTCCTGTCT | |
| | GCCACCATCCTCTATGAGATCTTGCTAGGGAAGGCCACCTTGTATGCCGTGCTGGTCAGTGCC | |
| | CTCGTGCTGATGGCCATGGTCAAGAGAAAGGATTCCAGAGGCTAG | |
| 207 | MSIRALFIFLWLQLDLVNGENVEQHPSTLSVQEGDSAVIKCTYSDSASNYFPWYKQELGKRPQ | TCR N full |
| | LIIDIRSNVGEKKDQRIAVTLNKTAKHFSLHITETQPEDSAVYFCAAIVGNQFYFGTGTSLTV | construct aa |
| | IPNIQKPDPAVYQLRDSKSSDKSVCLFTDFDSQTNVSQSKDSDVYITDKTVLDMRSMDFKSNS | |
| | AVAWSNKSDFACANAFNNSIIPEDTFFPSPESSCDVKLVEKSFETDTNLNFQNLSVIGFRILL | |
| | LKVAGFNLLMTLRLWSSGSGATNFSLLKQAGDVEENPGPMGTSLLCWMALCLLGADHADTGVS | |
| | QDPRHKITKRGQNVTFRCDPISEHNRLYWYRQTLGQGPEFLTYFQNEAQLEKSRLLSDRFSAE | |
| | RPKGSFSTLEIQRTEQGDSAMYLCASSETTLSEQFFGPGTRLTVLEDLKNVEPPEVAVFEPSE | |
| | AEISHTQKATLVCLATGFYPDHVELSWWVNGKEVHSGVSTDPQPLKEQPALNDSRYCLSSRLR | |
| | VSATFWQNPRNHFRCQVQFYGLSENDEWTQDRAKPVTQIVSAEAWGRADCGFTSESYQQGVLS | |
| | ATILYEILLGKATLYAVLVSALVLMAMVKRKDSRG | |
| 208 | AMRNNNDMR | TCR O Vα |
| | | CDR-3 |
| 209 | GENVEQHPSTLSVQEGDSAVIKCTYSDSASNYFPWYKQELGKRPQLIIDIRSNVGEKKDQRIA | TCR O Vα |
| | VTLNKTAKHFSLHITETQPEDSAVYFCAMRNNNDMRFGAGTRLTVKPN | (aa) |
| 210 | MSIRAVFIFLWLQLDLVNGENVEQHPSTLSVQEGDSAVIKCTYSDSASNYFPWYKQELGKRPQ | TCR O Vα + |
| | LIIDIRSNVGEKKDQRIAVTLNKTAKHFSLHITETQPEDSAVYFCAMRNNNDMRFGAGTRLTV | ss (aa) |
| | KPN | |
| 211 | MSIRAVFIFLWLQLDLVNGENVEQHPSTLSVQEGDSAVIKCTYSDSASNYFPWYKQELGKRPQ | TCR O Vα + |
| | LIIDIRSNVGEKKDQRIAVTLNKTAKHFSLHITETQPEDSAVYFCAMRNNNDMRFGAGTRLTV | Cα (aa) |
| | KPNIQNPDPAVYQLRDSKSSDKSVCLFTDFDSQTNVSQSKDSDVYITDKTVLDMRSMDFKSNS | |
| | AVAWSNKSDFACANAFNNSIIPEDTFFPSPESSCDVKLVEKSFETDTNLNFQNLSVIGFRILL | |
| | LKVAGFNLLMTLRLWSS | |
| 212 | ATGTCCATTCGAGCTGTATTTATATTCCTGTGGCTGCAGCTGGACTTGGTGAATGGAGAGAAT | TCR O Vα |
| | GTGGAGCAGCATCCTTCAACCCTGAGTGTCCAGGAGGGAGACAGCGCTGTTATCAAGTGTACT | (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAATGAGAAATAACAATGACATGCGCTTTGGAGCAGGGACCAGACTGACAGTA | |
| | AAACCAAAT | |
| 213 | ATGTCCATTCGAGCTGTATTTATATTCCTGTGGCTGCAGCTGGACTTGGTGAATGGAGAGAAT | TCR O Vα + |
| | GTGGAGCAGCATCCTTCAACCCTGAGTGTCCAGGAGGGAGACAGCGCTGTTATCAAGTGTACT | Cα (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAATGAGAAATAACAATGACATGCGCTTTGGAGCAGGGACCAGACTGACAGTA | |
| | AAACCAAATATCCAGAACCCTGACCCTGCCGTGTACCAGCTGAGAGACTCTAAATCCAGTGAC | |
| | AAGTCTGTCTGCCTATTCACCGATTTTGATTCTCAAACAAATGTGTCACAAAGTAAGGATTCT | |
| | GATGTGTATATCACAGACAAAACTGTGCTAGACATGAGGTCTATGGACTTCAAGAGCAACAGT | |
| | GCTGTGGCCTGGAGCAACAAATCTGACTTTGCATGTGCAAACGCCTTCAACAACAGCATTATT | |
| | CCAGAAGACACCTTCTTCCCCAGCCCAGAAAGTTCCTGTGATGTCAAGCTGGTCGAGAAAAGC | |
| | TTTGAAACAGATACGAACCTAAACTTTCAAAACCTGTCAGTGATTGGGTTCCGAATCCTCCTC | |
| | CTGAAAGTGGCCGGGTTTAATCTGCTCATGACGCTGCGGCTGTGGTCCAGC | |
| 214 | ASSLTTLDTQY | TCR O Vβ |
| | | CDR-3 |
| 215 | DTGVSQDPRHKITKRGQNVTFRCDPISEHNRLYWYRQTLGQGPEFLTYFQNEAQLEKSRLLSD | TCR O Vβ |
| | RFSAERPKGSFSTLEIQRTEQGDSAMYLCASSLTTLDTQYFGPGTRLTVL | (aa) |
| 216 | MGTSLLCWMALCLLGADHADTGVSQDPRHKITKRGQNVTFRCDPISEHNRLYWYRQTLGQGPE | TCR O Vβ + |
| | FLTYFQNEAQLEKSRLLSDRFSAERPKGSFSTLEIQRTEQGDSAMYLCASSLTTLDTQYFGPG | ss (aa) |
| | TRLTVL | |
| 217 | MGTSLLCWMALCLLGADHADTGVSQDPRHKITKRGQNVTFRCDPISEHNRLYWYRQTLGQGPE | TCR O Vβ + |
| | FLTYFQNEAQLEKSRLLSDRFSAERPKGSFSTLEIQRTEQGDSAMYLCASSLTTLDTQYFGPG | Cβ (aa) |
| | TRLTVLEDLKNVFPPEVAVFEPSEAEISHTQKATLVCLATGFYPDHVELSWWVNGKEVHSGVS | |
| | TDPQPLKEQPALNDSRYCLSSRLRVSATFWQNPRNHFRCQVQFYGLSENDEWTQDRAKPVTQI | |
| | VSAEAWGRADCGFTSESYQQGVLSATILYEILLGKATLYAVLVSALVLMAMVKRKDSRG | |
| 218 | ATGGGTACCAGCCTCCTCTGCTGGATGGCCCTGTGTCTCCTGGGGGCAGATCACGCAGATACT | TCR O Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAACAACATTGGATACGCAGTATTTTGGCCCAGGC | |
| | ACCCGGCTGACAGTGCTCGAG | |
| 219 | ATGGGTACCAGCCTCCTCTGCTGGATGGCCCTGTGTCTCCTGGGGGCAGATCACGCAGATACT | TCR O Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAACAACATTGGATACGCAGTATTTTGGCCCAGGC | |
| | ACCCGGCTGACAGTGCTCGAGGACCTGAAAAACGTGTTCCCACCCGAGGTCGCTGTGTTTGAG | |
| | CCATCAGAAGCAGAGATCTCCCACACCCAAAAGGCCACACTGGTATGCCTGGCCACAGGCTTC | |
| | TACCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCTGTCACCCAGATC | |
| | GTCAGCGCCGAGGCCTGGGGTAGAGCAGACTGTGGCTTCACCTCCGAGTCTTACCAGCAAGGG | |
| | GTCCTGTCTGCCACCATCCTCTATGAGATCTTGCTAGGGAAGGCCACCTTGTATGCCGTGCTG | |
| | GTCAGTGCCCTCGTGCTGATGGCCATGGTCAAGAGAAAGGATTCCAGAGGCTAG | |
| 220 | ATGTCCATTCGAGCTGTATTTATATTCCTGTGGCTGCAGCTGGACTTGGTGAATGGAGAGAAT | TCR O full |
| | GTGGAGCAGCATCCTTCAACCCTGAGTGTCCAGGAGGGAGACAGCGCTGTTATCAAGTGTACT | construct nt |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAATGAGAAATAACAATGACATGCGCTTTGGAGCAGGGACCAGACTGACAGTA | |
| | AAACCAAATATCCAGAACCCTGACCCTGCCGTGTACCAGCTGAGAGACTCTAAATCCAGTGAC | |
| | AAGTCTGTCTGCCTATTCACCGATTTTGATTCTCAAACAAATGTGTCACAAAGTAAGGATTCT | |
| | GATGTGTATATCACAGACAAAACTGTGCTAGACATGAGGTCTATGGACTTCAAGAGCAACAGT | |
| | GCTGTGGCCTGGAGCAACAAATCTGACTTTGCATGTGCAAACGCCTTCAACAACAGCATTATT | |
| | CCAGAAGACACCTTCTTCCCCAGCCCAGAAAGTTCCTGTGATGTCAAGCTGGTCGAGAAAAGC | |
| | TTTGAAACAGATACGAACCTAAACTTTCAAAACCTGTCAGTGATTGGGTTCCGAATCCTCCTC | |
| | CTGAAAGTGGCCGGGTTTAATCTGCTCATGACGCTGCGGCTGTGGTCCAGCGGCTCCGGAGCC | |
| | ACGAACTTCTCTCTGTTAAAGCAAGCAGGAGACGTGGAAGAAAACCCCGGTCCCATGGGTACC | |
| | AGCCTCCTCTGCTGGATGGCCCTGTGTCTCCTGGGGGCAGATCACGCAGATACTGGAGTCTCC | |
| | CAGGACCCCAGACACAAGATCACAAAGAGGGGACAGAATGTAACTTTCAGGTGTGATCCAATT | |
| | TCTGAACACAACCGCCTTTATTGGTACCGACAGACCCTGGGGCAGGGCCCAGAGTTTCTGACT | |
| | TACTTCCAGAATGAAGCTCAACTAGAAAAATCAAGGCTGCTCAGTGATCGGTTCTCTGCAGAG | |
| | AGGCCTAAGGGATCTTTCTCCACCTTGGAGATCCAGCGCACAGAGCAGGGGGACTCGGCCATG | |
| | TATCTCTGTGCCAGCAGCTTAACAACATTGGATACGCAGTATTTTGGCCCAGGCACCCGGCTG | |
| | ACAGTGCTCGAGGACCTGAAAAACGTGTTCCCACCCGAGGTCGCTGTGTTTGAGCCATCAGAA | |
| | GCAGAGATCTCCCACACCCAAAAGGCCACACTGGTATGCCTGGCCACAGGCTTCTACCCCGAC | |
| | CACGTGGAGCTGAGCTGGTGGGTGAATGGGAAGGAGGTGCACAGTGGGGTCAGCACAGACCCG | |
| | CAGCCCCTCAAGGAGCAGCCCGCCCTCAATGACTCCAGATACTGCCTGAGCAGCCGCCTGAGG | |
| | GTCTCGGCCACCTTCTGGCAGAACCCCCGCAACCACTTCCGCTGTCAAGTCCAGTTCTACGGG | |
| | CTCTCGGAGAATGACGAGTGGACCCAGGATAGGGCCAAACCTGTCACCCAGATCGTCAGCGCC | |
| | GAGGCCTGGGGTAGAGCAGACTGTGGCTTCACCTCCGAGTCTTACCAGCAAGGGGTCCTGTCT | |
| | GCCACCATCCTCTATGAGATCTTGCTAGGGAAGGCCACCTTGTATGCCGTGCTGGTCAGTGCC | |
| | CTCGTGCTGATGGCCATGGTCAAGAGAAAGGATTCCAGAGGCTAG | |
| 221 | MSIRAVFIFLWLQLDLVNGENVEQHPSTLSVQEGDSAVIKCTYSDSASNYFPWYKQELGKRPQ | TCR O full |
| | LIIDIRSNVGEKKDQRIAVTLNKTAKHFSLHITETQPEDSAVYFCAMRNNNDMRFGAGTRLTV | construct aa |
| | KPNIQNPDPAVYQLRDSKSSDKSVCLFTDFDSQTNVSQSKDSDVYITDKTVLDMRSMDFKSNS | |
| | AVAWSNKSDFACANAFNNSIIPEDTFFPSPESSCDVKLVEKSFETDTNLNFQNLSVIGFRILL | |
| | LKVAGFNLLMTLRLWSSGSGATNFSLLKQAGDVEENPGPMGTSLLCWMALCLLGADHADTGVS | |
| | QDPRHKITKRGQNVTFRCDPISEHNRLYWYRQTLGQGPEFLTYFQNEAQLEKSRLLSDRFSAE | |
| | RPKGSFSTLEIQRTEQGDSAMYLCASSLTTLDTQYFGPGTRLTVLEDLKNVEPPEVAVFEPSE | |
| | AEISHTQKATLVCLATGFYPDHVELSWWVNGKEVHSGVSTDPQPLKEQPALNDSRYCLSSRLR | |
| | VSATFWQNPRNHFRCQVQFYGLSENDEWTQDRAKPVTQIVSAEAWGRADCGFTSESYQQGVLS | |
| | ATILYEILLGKATLYAVLVSALVLMAMVKRKDSRG | |
| 222 | YGGTVN | TCR P Vα |
| | | CDR-1 |
| 223 | YFSGDPLV | TCR P Vα |
| | | CDR-2 |
| 224 | AVRDSGAGSYQLT | TCR P Vα |
| | | CDR-3 |
| 225 | AQSVSQHNHHVILSEAASLELGCNYSYGGTVNLFWYVQYPGQHLQLLLKYFSGDPLVKGIKGF | TCR P Vα |
| | EAEFIKSKFSFNLRKPSVQWSDTAEYFCAVRDSGAGSYQLTFGKGTKLSVIPN | (aa) |
| 226 | MLLLLIPVLGMIFALRDARAQSVSQHNHHVILSEAASLELGCNYSYGGTVNLFWYVQYPGQHL | TCR P Vα + |
| | QLLLKYFSGDPLVKGIKGFEAEFIKSKFSFNLRKPSVQWSDTAEYFCAVRDSGAGSYQLTFGK | ss (aa) |
| | GTKLSVIPN | |
| 227 | MLLLLIPVLGMIFALRDARAQSVSQHNHHVILSEAASLELGCNYSYGGTVNLFWYVQYPGQHL | TCR P Vα + |
| | QLLLKYFSGDPLVKGIKGFEAEFIKSKFSFNLRKPSVQWSDTAEYFCAVRDSGAGSYQLTFGK | Cα (aa) |
| | GTKLSVIPNIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGFNLLMTLRLWSS | |
| 228 | ATGCTCCTGTTGCTCATACCAGTGCTGGGGATGATTTTTGCCCTGAGAGATGCCAGAGCCCAG | TCR P Vα |
| | TCTGTGAGCCAGCATAACCACCACGTAATTCTCTCTGAAGCAGCCTCACTGGAGTTGGGATGC | (nt) |
| | AACTATTCCTATGGTGGAACTGTTAATCTCTTCTGGTATGTCCAGTACCCTGGTCAACACCTT | |
| | CAGCTTCTCCTCAAGTACTTTTCAGGGGATCCACTGGTTAAAGGCATCAAGGGCTTTGAGGCT | |
| | GAATTTATAAAGAGTAAATTCTCCTTTAATCTGAGGAAACCCTCTGTGCAGTGGAGTGACACA | |
| | GCTGAGTACTTCTGTGCCGTGAGGGACTCTGGGGCTGGGAGTTACCAACTCACTTTCGGGAAG | |
| | GGGACCAAACTCTCGGTCATACCAAAT | |
| 229 | ATGCTCCTGTTGCTCATACCAGTGCTGGGGATGATTTTTGCCCTGAGAGATGCCAGAGCCCAG | TCR P Vα + |
| | TCTGTGAGCCAGCATAACCACCACGTAATTCTCTCTGAAGCAGCCTCACTGGAGTTGGGATGC | Cα (nt) |
| | AACTATTCCTATGGTGGAACTGTTAATCTCTTCTGGTATGTCCAGTACCCTGGTCAACACCTT | |
| | CAGCTTCTCCTCAAGTACTTTTCAGGGGATCCACTGGTTAAAGGCATCAAGGGCTTTGAGGCT | |
| | GAATTTATAAAGAGTAAATTCTCCTTTAATCTGAGGAAACCCTCTGTGCAGTGGAGTGACACA | |
| | GCTGAGTACTTCTGTGCCGTGAGGGACTCTGGGGCTGGGAGTTACCAACTCACTTTCGGGAAG | |
| | GGGACCAAACTCTCGGTCATACCAAATATCCAGAACCCTGACCCTGCCGTGTACCAGCTGAGA | |
| | GACTCTAAATCCAGTGACAAGTCTGTCTGCCTATTCACCGATTTTGATTCTCAAACAAATGTG | |
| | TCACAAAGTAAGGATTCTGATGTGTATATCACAGACAAAACTGTGCTAGACATGAGGTCTATG | |
| | GACTTCAAGAGCAACAGTGCTGTGGCCTGGAGCAACAAATCTGACTTTGCATGTGCAAACGCC | |
| | TTCAACAACAGCATTATTCCAGAAGACACCTTCTTCCCCAGCCCAGAAAGTTCCTGTGATGTC | |
| | AAGCTGGTCGAGAAAAGCTTTGAAACAGATACGAACCTAAACTTTCAAAACCTGTCAGTGATT | |
| | GGGTTCCGAATCCTCCTCCTGAAAGTGGCCGGGTTTAATCTGCTCATGACGCTGCGGCTGTGG | |
| | TCCAGC | |
| 230 | ASLTGTVYNEQF | TCR P Vβ |
| | | CDR-3 |
| 231 | DTGVSQDPRHKITKRGQNVTFRCDPISEHNRLYWYRQTLGQGPEFLTYFQNEAQLEKSRLLSD | TCR P Vβ |
| | RFSAERPKGSFSTLEIQRTEQGDSAMYLCASLTGTVYNEQFFGPGTRLTVLE | (aa) |
| 232 | MGTSLLCWMALCLLGADHADTGVSQDPRHKITKRGQNVTFRCDPISEHNRLYWYRQTLGQGPE | TCR P Vβ + |
| | FLTYFQNEAQLEKSRLLSDRFSAERPKGSFSTLEIQRTEQGDSAMYLCASLTGTVYNEQFFGP | ss (aa) |
| | GTRLTVLE | |
| 233 | MGTSLLCWMALCLLGADHADTGVSQDPRHKITKRGQNVTFRCDPISEHNRLYWYRQTLGQGPE | TCR P Vβ + |
| | FLTYFQNEAQLEKSRLLSDRFSAERPKGSFSTLEIQRTEQGDSAMYLCASLTGTVYNEQFFGP | Cβ (aa) |
| | GTRLTVLEDLKNVFPPEVAVFEPSEAEISHTQKATLVCLATGFYPDHVELSWWVNGKEVHSGV | |
| | STDPQPLKEQPALNDSRYCLSSRLRVSATFWQNPRNHFRCQVQFYGLSENDEWTQDRAKPVTQ | |
| | IVSAEAWGRADCGFTSESYQQGVLSATILYEILLGKATLYAVLVSALVLMAMVKRKDSRG | |
| 234 | ATGGGTACCAGCCTCCTCTGCTGGATGGCCCTGTGTCTCCTGGGGGCAGATCACGCAGATACT | TCR P Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCCTTACCGGGACAGTCTACAATGAGCAGTTCTTCGGGCCA | |
| | GGGACACGGCTCACCGTGCTAGAG | |
| 235 | ATGGGTACCAGCCTCCTCTGCTGGATGGCCCTGTGTCTCCTGGGGGCAGATCACGCAGATACT | TCR P Vβ + |
| | GGAGTCTCCCAGGACCCCAGACACAAGATCACAAAGAGGGGACAGAATGTAACTTTCAGGTGT | Cβ (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCCTTACCGGGACAGTCTACAATGAGCAGTTCTTCGGGCCA | |
| | GGGACACGGCTCACCGTGCTAGAGGACCTGAAAAACGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 236 | ATGCTCCTGTTGCTCATACCAGTGCTGGGGATGATTTTTGCCCTGAGAGATGCCAGAGCCCAG | TCR P full |
| | TCTGTGAGCCAGCATAACCACCACGTAATTCTCTCTGAAGCAGCCTCACTGGAGTTGGGATGC | construct nt |
| | AACTATTCCTATGGTGGAACTGTTAATCTCTTCTGGTATGTCCAGTACCCTGGTCAACACCTT | |
| | CAGCTTCTCCTCAAGTACTTTTCAGGGGATCCACTGGTTAAAGGCATCAAGGGCTTTGAGGCT | |
| | GAATTTATAAAGAGTAAATTCTCCTTTAATCTGAGGAAACCCTCTGTGCAGTGGAGTGACACA | |
| | GCTGAGTACTTCTGTGCCGTGAGGGACTCTGGGGCTGGGAGTTACCAACTCACTTTCGGGAAG | |
| | GGGACCAAACTCTCGGTCATACCAAATATCCAGAACCCTGACCCTGCCGTGTACCAGCTGAGA | |
| | GACTCTAAATCCAGTGACAAGTCTGTCTGCCTATTCACCGATTTTGATTCTCAAACAAATGTG | |
| | TCACAAAGTAAGGATTCTGATGTGTATATCACAGACAAAACTGTGCTAGACATGAGGTCTATG | |
| | GACTTCAAGAGCAACAGTGCTGTGGCCTGGAGCAACAAATCTGACTTTGCATGTGCAAACGCC | |
| | TTCAACAACAGCATTATTCCAGAAGACACCTTCTTCCCCAGCCCAGAAAGTTCCTGTGATGTC | |
| | AAGCTGGTCGAGAAAAGCTTTGAAACAGATACGAACCTAAACTTTCAAAACCTGTCAGTGATT | |
| | GGGTTCCGAATCCTCCTCCTGAAAGTGGCCGGGTTTAATCTGCTCATGACGCTGCGGCTGTGG | |
| | TCCAGCGGCTCCGGAGCCACGAACTTCTCTCTGTTAAAGCAAGCAGGAGACGTGGAAGAAAAC | |
| | CCCGGTCCCATGGGTACCAGCCTCCTCTGCTGGATGGCCCTGTGTCTCCTGGGGGCAGATCAC | |
| | GCAGATACTGGAGTCTCCCAGGACCCCAGACACAAGATCACAAAGAGGGGACAGAATGTAACT | |
| | TTCAGGTGTGATCCAATTTCTGAACACAACCGCCTTTATTGGTACCGACAGACCCTGGGGCAG | |
| | GGCCCAGAGTTTCTGACTTACTTCCAGAATGAAGCTCAACTAGAAAAATCAAGGCTGCTCAGT | |
| | GATCGGTTCTCTGCAGAGAGGCCTAAGGGATCTTTCTCCACCTTGGAGATCCAGCGCACAGAG | |
| | CAGGGGGACTCGGCCATGTATCTCTGTGCCAGCCTTACCGGGACAGTCTACAATGAGCAGTTC | |
| | TTCGGGCCAGGGACACGGCTCACCGTGCTAGAGGACCTGAAAAACGTGTTCCCACCCGAGGTC | |
| | GCTGTGTTTGAGCCATCAGAAGCAGAGATCTCCCACACCCAAAAGGCCACACTGGTATGCCTG | |
| | GCCACAGGCTTCTACCCCGACCACGTGGAGCTGAGCTGGTGGGTGAATGGGAAGGAGGTGCAC | |
| | AGTGGGGTCAGCACAGACCCGCAGCCCCTCAAGGAGCAGCCCGCCCTCAATGACTCCAGATAC | |
| | TGCCTGAGCAGCCGCCTGAGGGTCTCGGCCACCTTCTGGCAGAACCCCCGCAACCACTTCCGC | |
| | TGTCAAGTCCAGTTCTACGGGCTCTCGGAGAATGACGAGTGGACCCAGGATAGGGCCAAACCT | |
| | GTCACCCAGATCGTCAGCGCCGAGGCCTGGGGTAGAGCAGACTGTGGCTTCACCTCCGAGTCT | |
| | TACCAGCAAGGGGTCCTGTCTGCCACCATCCTCTATGAGATCTTGCTAGGGAAGGCCACCTTG | |
| | TATGCCGTGCTGGTCAGTGCCCTCGTGCTGATGGCCATGGTCAAGAGAAAGGATTCCAGAGGC | |
| | TAG | |
| 237 | MLLLLIPVLGMIFALRDARAQSVSQHNHHVILSEAASLELGCNYSYGGTVNLFWYVQYPGQHL | TCR P full |
| | QLLLKYFSGDPLVKGIKGFEAEFIKSKFSFNLRKPSVQWSDTAEYFCAVRDSGAGSYQLTFGK | construct aa |
| | GTKLSVIPNIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGFNLLMTLRLWSSGSGATNFSLLKQAGDVEENPGPMGTSLLCWMALCLLGADH | |
| | ADTGVSQDPRHKITKRGQNVTFRCDPISEHNRLYWYRQTLGQGPEFLTYFQNEAQLEKSRLLS | |
| | DRFSAERPKGSFSTLEIQRTEQGDSAMYLCASLTGTVYNEQFFGPGTRLTVLEDLKNVFPPEV | |
| | AVFEPSEAEISHTQKATLVCLATGFYPDHVELSWWVNGKEVHSGVSTDPQPLKEQPALNDSRY | |
| | CLSSRLRVSATFWQNPRNHFRCQVQFYGLSENDEWTQDRAKPVTQIVSAEAWGRADCGFTSES | |
| | YQQGVLSATILYEILLGKATLYAVLVSALVLMAMVKRKDSRG | |
| 238 | AASNPTGANSKLT | TCR Q Vα |
| | | CDR-3 |
| 239 | GENVEQHPSTLSVQEGDSAVIKCTYSDSASNYFPWYKQELGKRPQLIIDIRSNVGEKKDQRIA | TCR Q Vα |
| | VTLNKTAKHFSLHITETQPEDSAVYFCAASNPTGANSKLTFGKGITLSVRPD | (aa) |
| 240 | MSIRAVFIFLWLQLDLVNGENVEQHPSTLSVQEGDSAVIKCTYSDSASNYFPWYKQELGKRPQ | TCR Q Vα + |
| | LIIDIRSNVGEKKDQRIAVTLNKTAKHFSLHITETQPEDSAVYFCAASNPTGANSKLTFGKGI | ss (aa) |
| | TLSVRPD | |
| 241 | MSIRAVFIFLWLQLDLVNGENVEQHPSTLSVQEGDSAVIKCTYSDSASNYFPWYKQELGKRPQ | TCR Q Vα + |
| | LIIDIRSNVGEKKDQRIAVTLNKTAKHFSLHITETQPEDSAVYFCAASNPTGANSKLTFGKGI | Cα (aa) |
| | TLSVRPDIQKPDPAVYQLRDSKSSDKSVCLFTDFDSQTNVSQSKDSDVYITDKTVLDMRSMDF | |
| | KSNSAVAWSNKSDFACANAFNNSIIPEDTFFPSPESSCDVKLVEKSFETDTNLNFQNLSVIGF | |
| | RILLLKVAGFNLLMTLRLWSS | |
| 242 | ATGTCCATTCGAGCTGTATTTATATTCCTGTGGCTGCAGCTGGACTTGGTGAATGGAGAGAAT | TCR Q Vα |
| | GTGGAGCAGCATCCTTCAACCCTGAGTGTCCAGGAGGGAGACAGCGCTGTTATCAAGTGTACT | (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCAAGTAACCCTACTGGAGCCAATAGTAAGCTGACATTTGGAAAAGGAATA | |
| | ACTCTGAGTGTTAGACCAGAT | |
| 243 | ATGTCCATTCGAGCTGTATTTATATTCCTGTGGCTGCAGCTGGACTTGGTGAATGGAGAGAAT | TCR Q Vα + |
| | GTGGAGCAGCATCCTTCAACCCTGAGTGTCCAGGAGGGAGACAGCGCTGTTATCAAGTGTACT | Cα (nt) |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCAAGTAACCCTACTGGAGCCAATAGTAAGCTGACATTTGGAAAAGGAATA | |
| | ACTCTGAGTGTTAGACCAGATATCCAGAAGCCTGACCCTGCCGTGTACCAGCTGAGAGACTCT | |
| | AAATCCAGTGACAAGTCTGTCTGCCTATTCACCGATTTTGATTCTCAAACAAATGTGTCACAA | |
| | AGTAAGGATTCTGATGTGTATATCACAGACAAAACTGTGCTAGACATGAGGTCTATGGACTTC | |
| | AACAGCATTATTCCAGAAGACACCTTCTTCCCCAGCCCAGAAAGTTCCTGTGATGTCAAGCTG | |
| | AAGAGCAACAGTGCTGTGGCCTGGAGCAACAAATCTGACTTTGCATGTGCAAACGCCTTCAAC | |
| | AACAGCATTATTCCAGAAGACACCTTCTTCCCCAGCCCAGAAAGTTCCTGTGATGTCAAGCTG | |
| | GTCGAGAAAAGCTTTGAAACAGATACGAACCTAAACTTTCAAAACCTGTCAGTGATTGGGTTC | |
| | CGAATCCTCCTCCTGAAAGTGGCCGGGTTTAATCTGCTCATGACGCTGCGGCTGTGGTCCAGC | |
| 244 | ASSLVRNEKLE | TCR Q Vβ |
| | | CDR-3 |
| 245 | DTGVSQDPRHKITKRGQNVTFRCDPISEHNRLYWYRQTLGQGPEFLTYFQNEAQLEKSRLLSD | TCR Q Vβ |
| | RFSAERPKGSFSTLEIQRTEQGDSAMYLCASSLVRNEKLFFGSGTQLSVLE | (aa) |
| 246 | MGTSLLCWMALCLLGADHADTGVSQDPRHKITKRGQNVTFRCDPISEHNRLYWYRQTLGQGPE | TCR Q Vβ + |
| | FLTYFQNEAQLEKSRLLSDRFSAERPKGSFSTLEIQRTEQGDSAMYLCASSLVRNEKLFFGSG | ss (aa) |
| | TQLSVLE | |
| 247 | MGTSLLCWMALCLLGADHADTGVSQDPRHKITKRGQNVTFRCDPISEHNRLYWYRQTLGQGPE | TCR Q Vβ + |
| | FLTYFQNEAQLEKSRLLSDRFSAERPKGSFSTLEIQRTEQGDSAMYLCASSLVRNEKLFFGSG | Cß (aa) |
| | TQLSVLEDLNKVFPPEVAVFEPSEAEISHTQKATLVCLATGFFPDHVELSWWVNGKEVHSGVS | |
| | TDPQPLKEQPALNDSRYCLSSRLRVSATFWQNPRNHERCQVQFYGLSENDEWTQDRAKPVTQI | |
| | VSAEAWGRADCGFTSVSYQQGVLSATILYEILLGKATLYAVLVSALVLMAMVKRKDF | |
| 248 | ATGGGTACCAGCCTCCTCTGCTGGATGGCCCTGTGTCTCCTGGGGGCAGATCACGCAGATACT | TCR Q Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGTCCGAAATGAAAAACTGTTTTTTGGCAGTGGA | |
| | ACCCAGCTCTCTGTCTTGGAG | |
| 249 | ATGGGTACCAGCCTCCTCTGCTGGATGGCCCTGTGTCTCCTGGGGGCAGATCACGCAGATACT | TCR Q Vβ + |
| | GGAGTCTCCCAGGACCCCAGACACAAGATCACAAAGAGGGGACAGAATGTAACTTTCAGGTGT | Cß (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAGTCCGAAATGAAAAACTGTTTTTTGGCAGTGGA | |
| | ACCCAGCTCTCTGTCTTGGAGGACCTGAACAAGGTGTTCCCACCCGAGGTCGCTGTGTTTGAG | |
| | CCATCAGAAGCAGAGATCTCCCACACCCAAAAGGCCACACTGGTGTGCCTGGCCACAGGCTTC | |
| | TTCCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCCGTCACCCAGATC | |
| | GTCAGCGCCGAGGCCTGGGGTAGAGCAGACTGTGGCTTTACCTCGGTGTCCTACCAGCAAGGG | |
| | GTCCTGTCTGCCACCATCCTCTATGAGATCCTGCTAGGGAAGGCCACCCTGTATGCTGTGCTG | |
| | GTCAGCGCCCTTGTGTTGATGGCCATGGTCAAGAGAAAGGATTTCTGA | |
| 250 | ATGTCCATTCGAGCTGTATTTATATTCCTGTGGCTGCAGCTGGACTTGGTGAATGGAGAGAAT | TCR Q full |
| | GTGGAGCAGCATCCTTCAACCCTGAGTGTCCAGGAGGGAGACAGCGCTGTTATCAAGTGTACT | construct nt |
| | TATTCAGACAGTGCCTCAAACTACTTCCCTTGGTATAAGCAAGAACTTGGAAAAAGACCTCAG | |
| | CTTATTATAGACATTCGTTCAAATGTGGGCGAAAAGAAAGACCAACGAATTGCTGTTACATTG | |
| | AACAAGACAGCCAAACATTTCTCCCTGCACATCACAGAGACCCAACCTGAAGACTCGGCTGTC | |
| | TACTTCTGTGCAGCAAGTAACCCTACTGGAGCCAATAGTAAGCTGACATTTGGAAAAGGAATA | |
| | ACTCTGAGTGTTAGACCAGATATCCAGAAGCCTGACCCTGCCGTGTACCAGCTGAGAGACTCT | |
| | AAATCCAGTGACAAGTCTGTCTGCCTATTCACCGATTTTGATTCTCAAACAAATGTGTCACAA | |
| | AGTAAGGATTCTGATGTGTATATCACAGACAAAACTGTGCTAGACATGAGGTCTATGGACTTC | |
| | AAGAGCAACAGTGCTGTGGCCTGGAGCAACAAATCTGACTTTGCATGTGCAAACGCCTTCAAC | |
| | AACAGCATTATTCCAGAAGACACCTTCTTCCCCAGCCCAGAAAGTTCCTGTGATGTCAAGCTG | |
| | GTCGAGAAAAGCTTTGAAACAGATACGAACCTAAACTTTCAAAACCTGTCAGTGATTGGGTTC | |
| | CGAATCCTCCTCCTGAAAGTGGCCGGGTTTAATCTGCTCATGACGCTGCGGCTGTGGTCCAGC | |
| | GGCTCCGGAGCCACGAACTTCTCTCTGTTAAAGCAAGCAGGAGACGTGGAAGAAAACCCCGGT | |
| | CCCATGGGTACCAGCCTCCTCTGCTGGATGGCCCTGTGTCTCCTGGGGGCAGATCACGCAGAT | |
| | ACTGGAGTCTCCCAGGACCCCAGACACAAGATCACAAAGAGGGGACAGAATGTAACTTTCAGG | |
| | TGTGATCCAATTTCTGAACACAACCGCCTTTATTGGTACCGACAGACCCTGGGGCAGGGCCCA | |
| | GAGTTTCTGACTTACTTCCAGAATGAAGCTCAACTAGAAAAATCAAGGCTGCTCAGTGATCGG | |
| | TTCTCTGCAGAGAGGCCTAAGGGATCTTTCTCCACCTTGGAGATCCAGCGCACAGAGCAGGGG | |
| | GACTCGGCCATGTATCTCTGTGCCAGCAGCTTAGTCCGAAATGAAAAACTGTTTTTTGGCAGT | |
| | GGAACCCAGCTCTCTGTCTTGGAGGACCTGAACAAGGTGTTCCCACCCGAGGTCGCTGTGTTT | |
| | GAGCCATCAGAAGCAGAGATCTCCCACACCCAAAAGGCCACACTGGTGTGCCTGGCCACAGGC | |
| | TTCTTCCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCCGTCACCCAG | |
| | ATCGTCAGCGCCGAGGCCTGGGGTAGAGCAGACTGTGGCTTTACCTCGGTGTCCTACCAGCAA | |
| | GGGGTCCTGTCTGCCACCATCCTCTATGAGATCCTGCTAGGGAAGGCCACCCTGTATGCTGTG | |
| | CTGGTCAGCGCCCTTGTGTTGATGGCCATGGTCAAGAGAAAGGATTTCTGA | |
| 251 | MSIRAVFIFLWLQLDLVNGENVEQHPSTLSVQEGDSAVIKCTYSDSASNYFPWYKQELGKRPQ | TCR Q full |
| | LIIDIRSNVGEKKDQRIAVTLNKTAKHFSLHITETQPEDSAVYFCAASNPTGANSKLTFGKGI | construct aa |
| | TLSVRPDIQKPDPAVYQLRDSKSSDKSVCLFTDFDSQTNVSQSKDSDVYITDKTVLDMRSMDF | |
| | KSNSAVAWSNKSDFACANAFNNSIIPEDTFFPSPESSCDVKLVEKSFETDTNLNFQNLSVIGF | |
| | RILLLKVAGFNLLMTLRLWSSGSGATNFSLLKQAGDVEENPGPMGTSLLCWMALCLLGADHAD | |
| | TGVSQDPRHKITKRGQNVTFRCDPISEHNRLYWYRQTLGQGPEFLTYFQNEAQLEKSRLLSDR | |
| | FSAERPKGSFSTLEIQRTEQGDSAMYLCASSLVRNEKLFFGSGTQLSVLEDLNKVFPPEVAVF | |
| | EPSEAEISHTQKATLVCLATGFFPDHVELSWWVNGKEVHSGVSTDPQPLKEQPALNDSRYCLS | |
| | SRLRVSATFWQNPRNHFRCQVQFYGLSENDEWTQDRAKPVTQIVSAEAWGRADCGFTSVSYQQ | |
| | GVLSATILYEILLGKATLYAVLVSALVLMAMVKRKDF | |
| 252 | ATEMNSNYQLI | TCR R Vα |
| | | CDR-3 |
| 253 | SQQGEEDPQALSIQEGENATMNCSYKTSINNLQWYRQNSGRGLVHLILIRSNEREKHSGRLRV | TCR R Vα |
| | TLDTSKKSSSLLITASRAADTASYFCATEMNSNYQLIWGAGTKLIIKPD | (aa) |
| 254 | METLLGVSLVILWLQLARVNSQQGEEDPQALSIQEGENATMNCSYKTSINNLQWYRQNSGRGL | TCR R Vα + |
| | VHLILIRSNEREKHSGRLRVTLDTSKKSSSLLITASRAADTASYFCATEMNSNYQLIWGAGTK | ss (aa) |
| | LIIKPD | |
| 255 | METLLGVSLVILWLQLARVNSQQGEEDPQALSIQEGENATMNCSYKTSINNLQWYRQNSGRGL | TCR R Vα + |
| | VHLILIRSNEREKHSGRLRVTLDTSKKSSSLLITASRAADTASYFCATEMNSNYQLIWGAGTK | Cα (aa) |
| | LIIKPDIQNPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGFR | |
| | ILLLKVAGFNLLMTLRLWSS | |
| 256 | ATGGAAACTCTCCTGGGAGTGTCTTTGGTGATTCTATGGCTTCAACTGGCTAGGGTGAACAGT | TCR R Vα |
| | CAACAGGGAGAAGAGGATCCTCAGGCCTTGAGCATCCAGGAGGGTGAAAATGCCACCATGAAC | (nt) |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACGGAAATGAATAGCAACTATCAGTTAATCTGGGGCGCTGGGACCAAG | |
| | CTAATTATAAAGCCAGAT | |
| 257 | ATGGAAACTCTCCTGGGAGTGTCTTTGGTGATTCTATGGCTTCAACTGGCTAGGGTGAACAGT | TCR R Vα + |
| | CAACAGGGAGAAGAGGATCCTCAGGCCTTGAGCATCCAGGAGGGTGAAAATGCCACCATGAAC | Cα (nt) |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACGGAAATGAATAGCAACTATCAGTTAATCTGGGGCGCTGGGACCAAG | |
| | CTAATTATAAAGCCAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATGTGTCACAAAGT | |
| | AAGGATTCTGATGTGTATATCACAGACAAAACTGTGCTAGACATGAGGTCTATGGACTTCAAG | |
| | AGCAACAGTGCTGTGGCCTGGAGCAACAAATCTGACTTTGCATGTGCAAACGCCTTCAACAAC | |
| | AGCATTATTCCAGAAGACACCTTCTTCCCCAGCCCAGAAAGTTCCTGTGATGTCAAGCTGGTC | |
| | GAGAAAAGCTTTGAAACAGATACGAACCTAAACTTTCAAAACCTGTCAGTGATTGGGTTCCGA | |
| | ATCCTCCTCCTGAAAGTGGCCGGGTTTAATCTGCTCATGACGCTGCGGCTGTGGTCCAGC | |
| 258 | ASSPLKGSNYGYT | TCR R Vβ |
| | | CDR-3 |
| 259 | DTGVSQDPRHKITKRGQNVTFRCDPISEHNRLYWYRQTLGQGPEFLTYFQNEAQLEKSRLLSD | TCR R Vβ |
| | RFSAERPKGSFSTLEIQRTEQGDSAMYLCASSPLKGSNYGYTFGSGTRLTVVE | (aa) |
| 260 | MGTSLLCWMALCLLGADHADTGVSQDPRHKITKRGQNVTFRCDPISEHNRLYWYRQTLGQGPE | TCR R Vβ + |
| | FLTYFQNEAQLEKSRLLSDRFSAERPKGSESTLEIQRTEQGDSAMYLCASSPLKGSNYGYTFG | ss (aa) |
| | SGTRLTVVE | |
| 261 | MGTSLLCWMALCLLGADHADTGVSQDPRHKITKRGQNVTFRCDPISEHNRLYWYRQTLGQGPE | TCR R Vβ + |
| | FLTYFQNEAQLEKSRLLSDRFSAERPKGSFSTLEIQRTEQGDSAMYLCASSPLKGSNYGYTFG | Cß (aa) |
| | SGTRLTVVEDLNKVFPPEVAVFEPSEAEISHTQKATLVCLATGFFPDHVELSWWVNGKEVHSG | |
| | VSTDPQPLKEQPALNDSRYCLSSRLRVSATFWQNPRNHFRCQVQFYGLSENDEWTQDRAKPVT | |
| | QIVSAEAWGRADCGFTSVSYQQGVLSATILYEILLGKATLYAVLVSALVLMAMVKRKDF | |
| 262 | ATGGGTACCAGCCTCCTCTGCTGGATGGCCCTGTGTCTCCTGGGGGCAGATCACGCAGATACT | TCR R Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCCCCCTAAAGGGCTCCAACTATGGCTACACCTTCGGT | |
| | TCGGGGACCAGGTTAACCGTTGTAGAG | |
| 263 | ATGGGTACCAGCCTCCTCTGCTGGATGGCCCTGTGTCTCCTGGGGGCAGATCACGCAGATACT | TCR R Vβ + |
| | GGAGTCTCCCAGGACCCCAGACACAAGATCACAAAGAGGGGACAGAATGTAACTTTCAGGTGT | Cß (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCCCCCTAAAGGGCTCCAACTATGGCTACACCTTCGGT | |
| | TCGGGGACCAGGTTAACCGTTGTAGAGGACCTGAACAAGGTGTTCCCACCCGAGGTCGCTGTG | |
| | TTTGAGCCATCAGAAGCAGAGATCTCCCACACCCAAAAGGCCACACTGGTGTGCCTGGCCACA | |
| | GGCTTCTTCCCCGACCACGTGGAGCTGAGCTGGTGGGTGAATGGGAAGGAGGTGCACAGTGGG | |
| | GTCAGCACAGACCCGCAGCCCCTCAAGGAGCAGCCCGCCCTCAATGACTCCAGATACTGCCTG | |
| | AGCAGCCGCCTGAGGGTCTCGGCCACCTTCTGGCAGAACCCCCGCAACCACTTCCGCTGTCAA | |
| | GTCCAGTTCTACGGGCTCTCGGAGAATGACGAGTGGACCCAGGATAGGGCCAAACCCGTCACC | |
| | CAGATCGTCAGCGCCGAGGCCTGGGGTAGAGCAGACTGTGGCTTTACCTCGGTGTCCTACCAG | |
| | CAAGGGGTCCTGTCTGCCACCATCCTCTATGAGATCCTGCTAGGGAAGGCCACCCTGTATGCT | |
| | GTGCTGGTCAGCGCCCTTGTGTTGATGGCCATGGTCAAGAGAAAGGATTTCTGA | |
| 264 | ATGGAAACTCTCCTGGGAGTGTCTTTGGTGATTCTATGGCTTCAACTGGCTAGGGTGAACAGT | TCR R full |
| | CAACAGGGAGAAGAGGATCCTCAGGCCTTGAGCATCCAGGAGGGTGAAAATGCCACCATGAAC | construct nt |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACGGAAATGAATAGCAACTATCAGTTAATCTGGGGCGCTGGGACCAAG | |
| | CTAATTATAAAGCCAGATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATGTGTCACAAAGT | |
| | AAGGATTCTGATGTGTATATCACAGACAAAACTGTGCTAGACATGAGGTCTATGGACTTCAAG | |
| | AGCAACAGTGCTGTGGCCTGGAGCAACAAATCTGACTTTGCATGTGCAAACGCCTTCAACAAC | |
| | AGCATTATTCCAGAAGACACCTTCTTCCCCAGCCCAGAAAGTTCCTGTGATGTCAAGCTGGTC | |
| | GAGAAAAGCTTTGAAACAGATACGAACCTAAACTTTCAAAACCTGTCAGTGATTGGGTTCCGA | |
| | ATCCTCCTCCTGAAAGTGGCCGGGTTTAATCTGCTCATGACGCTGCGGCTGTGGTCCAGCGGC | |
| | TCCGGAGCCACGAACTTCTCTCTGTTAAAGCAAGCAGGAGACGTGGAAGAAAACCCCGGTCCC | |
| | ATGGGTACCAGCCTCCTCTGCTGGATGGCCCTGTGTCTCCTGGGGGCAGATCACGCAGATACT | |
| | GGAGTCTCCCAGGACCCCAGACACAAGATCACAAAGAGGGGACAGAATGTAACTTTCAGGTGT | |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCCCCCTAAAGGGCTCCAACTATGGCTACACCTTCGGT | |
| | TCGGGGACCAGGTTAACCGTTGTAGAGGACCTGAACAAGGTGTTCCCACCCGAGGTCGCTGTG | |
| | TTTGAGCCATCAGAAGCAGAGATCTCCCACACCCAAAAGGCCACACTGGTGTGCCTGGCCACA | |
| | GGCTTCTTCCCCGACCACGTGGAGCTGAGCTGGTGGGTGAATGGGAAGGAGGTGCACAGTGGG | |
| | GTCAGCACAGACCCGCAGCCCCTCAAGGAGCAGCCCGCCCTCAATGACTCCAGATACTGCCTG | |
| | AGCAGCCGCCTGAGGGTCTCGGCCACCTTCTGGCAGAACCCCCGCAACCACTTCCGCTGTCAA | |
| | GTCCAGTTCTACGGGCTCTCGGAGAATGACGAGTGGACCCAGGATAGGGCCAAACCCGTCACC | |
| | CAGATCGTCAGCGCCGAGGCCTGGGGTAGAGCAGACTGTGGCTTTACCTCGGTGTCCTACCAG | |
| | CAAGGGGTCCTGTCTGCCACCATCCTCTATGAGATCCTGCTAGGGAAGGCCACCCTGTATGCT | |
| | GTGCTGGTCAGCGCCCTTGTGTTGATGGCCATGGTCAAGAGAAAGGATTTCTGA | |
| 265 | METLLGVSLVILWLQLARVNSQQGEEDPQALSIQEGENATMNCSYKTSINNLQWYRQNSGRGL | TCR R full |
| | VHLILIRSNEREKHSGRLRVTLDTSKKSSSLLITASRAADTASYFCATEMNSNYQLIWGAGTK | construct aa |
| | LIIKPDIQNPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGER | |
| | ILLLKVAGFNLLMTLRLWSSGSGATNFSLLKQAGDVEENPGPMGTSLLCWMALCLLGADHADT | |
| | GVSQDPRHKITKRGQNVTFRCDPISEHNRLYWYRQTLGQGPEFLTYFQNEAQLEKSRLLSDRF | |
| | SAERPKGSFSTLEIQRTEQGDSAMYLCASSPLKGSNYGYTFGSGTRLTVVEDLNKVEPPEVAV | |
| | FEPSEAEISHTQKATLVCLATGFFPDHVELSWWVNGKEVHSGVSTDPQPLKEQPALNDSRYCL | |
| | SSRLRVSATFWQNPRNHFRCQVQFYGLSENDEWTQDRAKPVTQIVSAEAWGRADCGFTSVSYQ | |
| | QGVLSATILYEILLGKATLYAVLVSALVLMAMVKRKDF | |
| 266 | TISGNEY | TCR S Vα |
| | | CDR-1 |
| 267 | GLKNN | TCR S Vα |
| | | CDR-2 |
| 268 | IVRASTSGTYKYI | TCR S Vα |
| | | CDR-3 |
| 269 | DAKTTQPPSMDCAEGRAANLPCNHSTISGNEYVYWYRQIHSQGPQYIIHGLKNNETNEMASLI | TCR S Vα |
| | ITEDRKSSTLILPHATLRDTAVYYCIVRASTSGTYKYIFGTGTRLKVLAN | (aa) |
| 270 | MRLVARVTVFLTFGTIIDAKTTQPPSMDCAEGRAANLPCNHSTISGNEYVYWYRQIHSQGPQY | TCR S Vα + |
| | IIHGLKNNETNEMASLIITEDRKSSTLILPHATLRDTAVYYCIVRASTSGTYKYIFGTGTRLK | ss (aa) |
| | VLAN | |
| 271 | MRLVARVTVFLTFGTIIDAKTTQPPSMDCAEGRAANLPCNHSTISGNEYVYWYRQIHSQGPQY | TCR S Vα + |
| | IIHGLKNNETNEMASLIITEDRKSSTLILPHATLRDTAVYYCIVRASTSGTYKYIFGTGTRLK | Cα (aa) |
| | VLANIQKPDPAVYQLRDSKSSDKSVCLFTDFDSQTNVSQSKDSDVYITDKTVLDMRSMDFKSN | |
| | SAVAWSNKSDFACANAFNNSIIPEDTFFPSPESSCDVKLVEKSFETDTNLNFQNLSVIGFRIL | |
| | LLKVAGFNLLMTLRLWSS | |
| 272 | ATGAGGCTGGTGGCAAGAGTAACTGTGTTTCTGACCTTTGGAACTATAATTGATGCTAAGACC | TCR S Vα |
| | ACCCAGCCCCCCTCCATGGATTGCGCTGAAGGAAGAGCTGCAAACCTGCCTTGTAATCACTCT | (nt) |
| | ACCATCAGTGGAAATGAGTATGTGTATTGGTATCGACAGATTCACTCCCAGGGGCCACAGTAT | |
| | ATCATTCATGGTCTAAAAAACAATGAAACCAATGAAATGGCCTCTCTGATCATCACAGAAGAC | |
| | AGAAAGTCCAGCACCTTGATCCTGCCCCACGCTACGCTGAGAGACACTGCTGTGTACTATTGC | |
| | ATCGTCAGAGCGTCTACCTCAGGAACCTACAAATACATCTTTGGAACAGGCACCAGGCTGAAG | |
| | GTTTTAGCAAAT | |
| 273 | ATGAGGCTGGTGGCAAGAGTAACTGTGTTTCTGACCTTTGGAACTATAATTGATGCTAAGACC | TCR S Vα + |
| | ACCCAGCCCCCCTCCATGGATTGCGCTGAAGGAAGAGCTGCAAACCTGCCTTGTAATCACTCT | Cα (nt) |
| | ACCATCAGTGGAAATGAGTATGTGTATTGGTATCGACAGATTCACTCCCAGGGGCCACAGTAT | |
| | ATCATTCATGGTCTAAAAAACAATGAAACCAATGAAATGGCCTCTCTGATCATCACAGAAGAC | |
| | AGAAAGTCCAGCACCTTGATCCTGCCCCACGCTACGCTGAGAGACACTGCTGTGTACTATTGC | |
| | ATCGTCAGAGCGTCTACCTCAGGAACCTACAAATACATCTTTGGAACAGGCACCAGGCTGAAG | |
| | GTTTTAGCAAATATCCAGAAGCCTGACCCTGCCGTGTACCAGCTGAGAGACTCTAAATCCAGT | |
| | GACAAGTCTGTCTGCCTATTCACCGATTTTGATTCTCAAACAAATGTGTCACAAAGTAAGGAT | |
| | TCTGATGTGTATATCACAGACAAAACTGTGCTAGACATGAGGTCTATGGACTTCAAGAGCAAC | |
| | AGTGCTGTGGCCTGGAGCAACAAATCTGACTTTGCATGTGCAAACGCCTTCAACAACAGCATT | |
| | ATTCCAGAAGACACCTTCTTCCCCAGCCCAGAAAGTTCCTGTGATGTCAAGCTGGTCGAGAAA | |
| | AGCTTTGAAACAGATACGAACCTAAACTTTCAAAACCTGTCAGTGATTGGGTTCCGAATCCTC | |
| | CTCCTGAAAGTGGCCGGGTTTAATCTGCTCATGACGCTGCGGCTGTGGTCCAGC | |
| 274 | ATGGGTACCAGCCTCCTCTGCTGGATGGCCCTGTGTCTCCTGGGGGCAGATCACGCAGATACT | TCR S Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGTCCCGGGACAGTCTACAATGAGCAGTTCTTCGGGCCA | |
| | GGGACACGGCTCACCGTGCTAGAG | |
| 275 | ATGGGTACCAGCCTCCTCTGCTGGATGGCCCTGTGTCTCCTGGGGGCAGATCACGCAGATACT | TCR S Vβ + |
| | GGAGTCTCCCAGGACCCCAGACACAAGATCACAAAGAGGGGACAGAATGTAACTTTCAGGTGT | Cß (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGTCCCGGGACAGTCTACAATGAGCAGTTCTTCGGGCCA | |
| | GGGACACGGCTCACCGTGCTAGAGGACCTGAAAAACGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 276 | ATGAGGCTGGTGGCAAGAGTAACTGTGTTTCTGACCTTTGGAACTATAATTGATGCTAAGACC | TCR S full |
| | ACCCAGCCCCCCTCCATGGATTGCGCTGAAGGAAGAGCTGCAAACCTGCCTTGTAATCACTCT | construct nt |
| | ACCATCAGTGGAAATGAGTATGTGTATTGGTATCGACAGATTCACTCCCAGGGGCCACAGTAT | |
| | ATCATTCATGGTCTAAAAAACAATGAAACCAATGAAATGGCCTCTCTGATCATCACAGAAGAC | |
| | AGAAAGTCCAGCACCTTGATCCTGCCCCACGCTACGCTGAGAGACACTGCTGTGTACTATTGC | |
| | ATCGTCAGAGCGTCTACCTCAGGAACCTACAAATACATCTTTGGAACAGGCACCAGGCTGAAG | |
| | GTTTTAGCAAATATCCAGAAGCCTGACCCTGCCGTGTACCAGCTGAGAGACTCTAAATCCAGT | |
| | GACAAGTCTGTCTGCCTATTCACCGATTTTGATTCTCAAACAAATGTGTCACAAAGTAAGGAT | |
| | TCTGATGTGTATATCACAGACAAAACTGTGCTAGACATGAGGTCTATGGACTTCAAGAGCAAC | |
| | AGTGCTGTGGCCTGGAGCAACAAATCTGACTTTGCATGTGCAAACGCCTTCAACAACAGCATT | |
| | ATTCCAGAAGACACCTTCTTCCCCAGCCCAGAAAGTTCCTGTGATGTCAAGCTGGTCGAGAAA | |
| | AGCTTTGAAACAGATACGAACCTAAACTTTCAAAACCTGTCAGTGATTGGGTTCCGAATCCTC | |
| | CTCCTGAAAGTGGCCGGGTTTAATCTGCTCATGACGCTGCGGCTGTGGTCCAGCGGCTCCGGA | |
| | GCCACGAACTTCTCTCTGTTAAAGCAAGCAGGAGACGTGGAAGAAAACCCCGGTCCCATGGGT | |
| | ACCAGCCTCCTCTGCTGGATGGCCCTGTGTCTCCTGGGGGCAGATCACGCAGATACTGGAGTC | |
| | TCCCAGGACCCCAGACACAAGATCACAAAGAGGGGACAGAATGTAACTTTCAGGTGTGATCCA | |
| | ATTTCTGAACACAACCGCCTTTATTGGTACCGACAGACCCTGGGGCAGGGCCCAGAGTTTCTG | |
| | ACTTACTTCCAGAATGAAGCTCAACTAGAAAAATCAAGGCTGCTCAGTGATCGGTTCTCTGCA | |
| | GAGAGGCCTAAGGGATCTTTCTCCACCTTGGAGATCCAGCGCACAGAGCAGGGGGACTCGGCC | |
| | ATGTATCTCTGTGCCAGCAGTCCCGGGACAGTCTACAATGAGCAGTTCTTCGGGCCAGGGACA | |
| | CGGCTCACCGTGCTAGAGGACCTGAAAAACGTGTTCCCACCCGAGGTCGCTGTGTTTGAGCCA | |
| | TCAGAAGCAGAGATCTCCCACACCCAAAAGGCCACACTGGTATGCCTGGCCACAGGCTTCTAC | |
| | CCCGACCACGTGGAGCTGAGCTGGTGGGTGAATGGGAAGGAGGTGCACAGTGGGGTCAGCACA | |
| | GACCCGCAGCCCCTCAAGGAGCAGCCCGCCCTCAATGACTCCAGATACTGCCTGAGCAGCCGC | |
| | CTGAGGGTCTCGGCCACCTTCTGGCAGAACCCCCGCAACCACTTCCGCTGTCAAGTCCAGTTC | |
| | TACGGGCTCTCGGAGAATGACGAGTGGACCCAGGATAGGGCCAAACCTGTCACCCAGATCGTC | |
| | AGCGCCGAGGCCTGGGGTAGAGCAGACTGTGGCTTCACCTCCGAGTCTTACCAGCAAGGGGTC | |
| | CTGTCTGCCACCATCCTCTATGAGATCTTGCTAGGGAAGGCCACCTTGTATGCCGTGCTGGTC | |
| | AGTGCCCTCGTGCTGATGGCCATGGTCAAGAGAAAGGATTCCAGAGGCTAG | |
| 277 | MRLVARVTVFLTFGTIIDAKTTQPPSMDCAEGRAANLPCNHSTISGNEYVYWYRQIHSQGPQY | TCR S full |
| | IIHGLKNNETNEMASLIITEDRKSSTLILPHATLRDTAVYYCIVRASTSGTYKYIFGTGTRLK | construct aa |
| | VLANIQKPDPAVYQLRDSKSSDKSVCLFTDFDSQTNVSQSKDSDVYITDKTVLDMRSMDFKSN | |
| | SAVAWSNKSDFACANAFNNSIIPEDTFFPSPESSCDVKLVEKSFETDTNLNFQNLSVIGFRIL | |
| | LLKVAGFNLLMTLRLWSSGSGATNFSLLKQAGDVEENPGPMGTSLLCWMALCLLGADHADTGV | |
| | SQDPRHKITKRGQNVTFRCDPISEHNRLYWYRQTLGQGPEFLTYFQNEAQLEKSRLLSDRESA | |
| | ERPKGSFSTLEIQRTEQGDSAMYLCASSPGTVYNEQFFGPGTRLTVLEDLKNVFPPEVAVFEP | |
| | SEAEISHTQKATLVCLATGFYPDHVELSWWVNGKEVHSGVSTDPQPLKEQPALNDSRYCLSSR | |
| 278 | ATPSGNTPLV | TCR T Vα |
| | | CDR-3 |
| 279 | SQQGEEDPQALSIQEGENATMNCSYKTSINNLQWYRQNSGRGLVHLILIRSNEREKHSGRLRV | TCR T Vα |
| | TLDTSKKSSSLLITASRAADTASYFCATPSGNTPLVFGKGTRLSVIAN | (aa) |
| 280 | METLLGVSLVILWLQLARVNSQQGEEDPQALSIQEGENATMNCSYKTSINNLQWYRQNSGRGL | TCR T Vα + |
| | VHLILIRSNEREKHSGRLRVTLDTSKKSSSLLITASRAADTASYFCATPSGNTPLVFGKGTRL | ss (aa) |
| | SVIAN | |
| 281 | METLLGVSLVILWLQLARVNSQQGEEDPQALSIQEGENATMNCSYKTSINNLQWYRQNSGRGL | TCR T Vα + |
| | VHLILIRSNEREKHSGRLRVTLDTSKKSSSLLITASRAADTASYFCATPSGNTPLVFGKGTRL | Cα (aa) |
| | SVIANIQNPDPAVYQLRDSKSSDKSVCLFTDFDSQTNVSQSKDSDVYITDKTVLDMRSMDFKS | |
| | NSAVAWSNKSDFACANAFNNSIIPEDTFFPSPESSCDVKLVEKSFETDTNLNFQNLSVIGFRI | |
| | LLLKVAGFNLLMTLRLWSS | |
| 282 | ATGGAAACTCTCCTGGGAGTGTCTTTGGTGATTCTATGGCTTCAACTGGCTAGGGTGAACAGT | TCR T Vα |
| | CAACAGGGAGAAGAGGATCCTCAGGCCTTGAGCATCCAGGAGGGTGAAAATGCCACCATGAAC | (nt) |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACCCCTTCAGGAAACACACCTCTTGTCTTTGGAAAGGGCACAAGACTT | |
| | TCTGTGATTGCAAAT | |
| 283 | ATGGAAACTCTCCTGGGAGTGTCTTTGGTGATTCTATGGCTTCAACTGGCTAGGGTGAACAGT | TCR T Vα + |
| | CAACAGGGAGAAGAGGATCCTCAGGCCTTGAGCATCCAGGAGGGTGAAAATGCCACCATGAAC | Cα (nt) |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACCCCTTCAGGAAACACACCTCTTGTCTTTGGAAAGGGCACAAGACTT | |
| | TCTGTGATTGCAAATATCCAGAACCCTGACCCTGCCGTGTACCAGCTGAGAGACTCTAAATCC | |
| | AGTGACAAGTCTGTCTGCCTATTCACCGATTTTGATTCTCAAACAAATGTGTCACAAAGTAAG | |
| | GATTCTGATGTGTATATCACAGACAAAACTGTGCTAGACATGAGGTCTATGGACTTCAAGAGC | |
| | AACAGTGCTGTGGCCTGGAGCAACAAATCTGACTTTGCATGTGCAAACGCCTTCAACAACAGC | |
| | ATTATTCCAGAAGACACCTTCTTCCCCAGCCCAGAAAGTTCCTGTGATGTCAAGCTGGTCGAG | |
| | AAAAGCTTTGAAACAGATACGAACCTAAACTTTCAAAACCTGTCAGTGATTGGGTTCCGAATC | |
| | CTCCTCCTGAAAGTGGCCGGGTTTAATCTGCTCATGACGCTGCGGCTGTGGTCCAGC | |
| 284 | ASSQLAGVIEQF | TCR T Vβ |
| | | CDR-3 |
| 285 | DTGVSQDPRHKITKRGQNVTFRCDPISEHNRLYWYRQTLGQGPELLTYFQNEAQLEKSRLLSD | TCR T Vβ |
| | RFSAERPKGSFSTLEIQRTEQGDSAMYLCASSQLAGVIEQFFGPGTRLTVLE | (aa) |
| 286 | MGTSLLCWMALCLLGADHADTGVSQDPRHKITKRGQNVTFRCDPISEHNRLYWYRQTLGQGPE | TCR T Vβ + |
| | LLTYFQNEAQLEKSRLLSDRFSAERPKGSESTLEIQRTEQGDSAMYLCASSQLAGVIEQFFGP | ss (aa) |
| | GTRLTVLE | |
| 287 | MGTSLLCWMALCLLGADHADTGVSQDPRHKITKRGQNVTFRCDPISEHNRLYWYRQTLGQGPE | TCR T Vβ + |
| | FLTYFQNEAQLEKSRLLSDRFSAERPKGSFSTLEIQRTEQGDSAMYLCASSQLAGVIEQFFGP | Cß (aa) |
| | GTRLTVLEDLKNVFPPEVAVFEPSEAEISHTQKATLVCLATGFYPDHVELSWWVNGKEVHSGV | |
| | STDPQPLKEQPALNDSRYCLSSRLRVSATFWQNPRNHFRCQVQFYGLSENDEWTQDRAKPVTQ | |
| | IVSAEAWGRADCGFTSESYQQGVLSATILYEILLGKATLYAVLVSALVLMAMVKRKDSRG | |
| 288 | ATGGGTACCAGCCTCCTCTGCTGGATGGCCCTGTGTCTCCTGGGGGCAGATCACGCAGATACT | TCR T Vβ |
| | GGAGTCTCCCAGGACCCCAGACACAAGATCACAAAGAGGGGACAGAATGTAACTTTCAGGTGT | (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCTCCCAACTAGCGGGAGTGATTGAGCAGTTCTTCGGGCCA | |
| | GGGACACGGCTCACCGTGCTAGAG | |
| 289 | ATGGGTACCAGCCTCCTCTGCTGGATGGCCCTGTGTCTCCTGGGGGCAGATCACGCAGATACT | TCR T Vβ + |
| | GGAGTCTCCCAGGACCCCAGACACAAGATCACAAAGAGGGGACAGAATGTAACTTTCAGGTGT | Cß (nt) |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCTCCCAACTAGCGGGAGTGATTGAGCAGTTCTTCGGGCCA | |
| | GGGACACGGCTCACCGTGCTAGAGGACCTGAAAAACGTGTTCCCACCCGAGGTCGCTGTGTTT | |
| | GAGCCATCAGAAGCAGAGATCTCCCACACCCAAAAGGCCACACTGGTATGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTAG | |
| 290 | ATGGAAACTCTCCTGGGAGTGTCTTTGGTGATTCTATGGCTTCAACTGGCTAGGGTGAACAGT | TCR T full |
| | CAACAGGGAGAAGAGGATCCTCAGGCCTTGAGCATCCAGGAGGGTGAAAATGCCACCATGAAC | construct nt |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACCCCTTCAGGAAACACACCTCTTGTCTTTGGAAAGGGCACAAGACTT | |
| | TCTGTGATTGCAAATATCCAGAACCCTGACCCTGCCGTGTACCAGCTGAGAGACTCTAAATCC | |
| | AGTGACAAGTCTGTCTGCCTATTCACCGATTTTGATTCTCAAACAAATGTGTCACAAAGTAAG | |
| | GATTCTGATGTGTATATCACAGACAAAACTGTGCTAGACATGAGGTCTATGGACTTCAAGAGC | |
| | AACAGTGCTGTGGCCTGGAGCAACAAATCTGACTTTGCATGTGCAAACGCCTTCAACAACAGC | |
| | ATTATTCCAGAAGACACCTTCTTCCCCAGCCCAGAAAGTTCCTGTGATGTCAAGCTGGTCGAG | |
| | AAAAGCTTTGAAACAGATACGAACCTAAACTTTCAAAACCTGTCAGTGATTGGGTTCCGAATC | |
| | CTCCTCCTGAAAGTGGCCGGGTTTAATCTGCTCATGACGCTGCGGCTGTGGTCCAGCGGCTCC | |
| | GGAGCCACGAACTTCTCTCTGTTAAAGCAAGCAGGAGACGTGGAAGAAAACCCCGGTCCCATG | |
| | GGTACCAGCCTCCTCTGCTGGATGGCCCTGTGTCTCCTGGGGGCAGATCACGCAGATACTGGA | |
| | GTCTCCCAGGACCCCAGACACAAGATCACAAAGAGGGGACAGAATGTAACTTTCAGGTGTGAT | |
| | CCAATTTCTGAACACAACCGCCTTTATTGGTACCGACAGACCCTGGGGCAGGGCCCAGAGTTT | |
| | CTGACTTACTTCCAGAATGAAGCTCAACTAGAAAAATCAAGGCTGCTCAGTGATCGGTTCTCT | |
| | GCAGAGAGGCCTAAGGGATCTTTCTCCACCTTGGAGATCCAGCGCACAGAGCAGGGGGACTCG | |
| | GCCATGTATCTCTGTGCCAGCTCCCAACTAGCGGGAGTGATTGAGCAGTTCTTCGGGCCAGGG | |
| | ACACGGCTCACCGTGCTAGAGGACCTGAAAAACGTGTTCCCACCCGAGGTCGCTGTGTTTGAG | |
| | CCATCAGAAGCAGAGATCTCCCACACCCAAAAGGCCACACTGGTATGCCTGGCCACAGGCTTC | |
| | TACCCCGACCACGTGGAGCTGAGCTGGTGGGTGAATGGGAAGGAGGTGCACAGTGGGGTCAGC | |
| | ACAGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCTGTCACCCAGATC | |
| | GTCAGCGCCGAGGCCTGGGGTAGAGCAGACTGTGGCTTCACCTCCGAGTCTTACCAGCAAGGG | |
| | GTCCTGTCTGCCACCATCCTCTATGAGATCTTGCTAGGGAAGGCCACCTTGTATGCCGTGCTG | |
| | GTCAGTGCCCTCGTGCTGATGGCCATGGTCAAGAGAAAGGATTCCAGAGGCTAG | |
| 291 | METLLGVSLVILWLQLARVNSQQGEEDPQALSIQEGENATMNCSYKTSINNLQWYRQNSGRGL | TCR T full |
| | VHLILIRSNEREKHSGRLRVTLDTSKKSSSLLITASRAADTASYFCATPSGNTPLVFGKGTRL | construct aa |
| | SVIANIQNPDPAVYQLRDSKSSDKSVCLFTDFDSQTNVSQSKDSDVYITDKTVLDMRSMDFKS | |
| | NSAVAWSNKSDFACANAFNNSIIPEDTFFPSPESSCDVKLVEKSFETDTNLNFQNLSVIGFRI | |
| | LLLKVAGFNLLMTLRLWSSGSGATNFSLLKQAGDVEENPGPMGTSLLCWMALCLLGADHADTG | |
| | VSQDPRHKITKRGQNVTFRCDPISEHNRLYWYRQTLGQGPEFLTYFQNEAQLEKSRLLSDRES | |
| | AERPKGSFSTLEIQRTEQGDSAMYLCASSQLAGVIEQFFGPGTRLTVLEDLKNVFPPEVAVFE | |
| | PSEAEISHTQKATLVCLATGFYPDHVELSWWVNGKEVHSGVSTDPQPLKEQPALNDSRYCLSS | |
| | RLRVSATFWQNPRNHFRCQVQFYGLSENDEWTQDRAKPVTQIVSAEAWGRADCGFTSESYQQG | |
| | VLSATILYEILLGKATLYAVLVSALVLMAMVKRKDSRG | |
| 292 | GGCTCCGGA | Linker (nt) |
| 293 | ATATCCAGAACCCTGACCCTGCCGTGTACCAGCTGAGAGACTCTAAATCCAGTGACAAGTCTG | TRAC allele |
| | TCTGCCTATTCACCGATTTTGATTCTCAAACAAATGTGTCACAAAGTAAGGATTCTGATGTGT | 1 (nt) |
| | ATATCACAGACAAAACTGTGCTAGACATGAGGTCTATGGACTTCAAGAGCAACAGTGCTGTGG | |
| | CCTGGAGCAACAAATCTGACTTTGCATGTGCAAACGCCTTCAACAACAGCATTATTCCAGAAG | |
| | ACACCTTCTTCCCCAGCCCAGAAAGTTCCTGTGATGTCAAGCTGGTCGAGAAAAGCTTTGAAA | |
| | CAGATACGAACCTAAACTTTCAAAACCTGTCAGTGATTGGGTTCCGAATCCTCCTCCTGAAAG | |
| | TGGCCGGGTTTAATCTGCTCATGACGCTGCGGCTGTGGTCCAGC | |
| 294 | IQNPDPAVYQLRDSKSSDKSVCLFTDFDSQTNVSQSKDSDVYITDKTVLDMRSMDFKSNSAVA | TRAC allele |
| | WSNKSDFACANAFNNSIIPEDTFFPSPESSCDVKLVEKSFETDTNLNFQNLSVIGFRILLLKV | 1 (aa) |
| | AGFNLLMTLRLWSS | |
| 295 | ATCCAGAAGCCTGACCCTGCCGTGTACCAGCTGAGAGACTCTAAATCCAGTGACAAGTCTGTC | TRAC allele |
| | TGCCTATTCACCGATTTTGATTCTCAAACAAATGTGTCACAAAGTAAGGATTCTGATGTGTAT | 2 (nt) |
| | ATCACAGACAAAACTGTGCTAGACATGAGGTCTATGGACTTCAAGAGCAACAGTGCTGTGGCC | |
| | TGGAGCAACAAATCTGACTTTGCATGTGCAAACGCCTTCAACAACAGCATTATTCCAGAAGAC | |
| | ACCTTCTTCCCCAGCCCAGAAAGTTCCTGTGATGTCAAGCTGGTCGAGAAAAGCTTTGAAACA | |
| | GATACGAACCTAAACTTTCAAAACCTGTCAGTGATTGGGTTCCGAATCCTCCTCCTGAAAGTG | |
| | GCCGGGTTTAATCTGCTCATGACGCTGCGGCTGTGGTCCAGC | |
| 296 | IQKPDPAVYQLRDSKSSDKSVCLFTDFDSQTNVSQSKDSDVYITDKTVLDMRSMDFKSNSAVA | TRAC allele |
| | WSNKSDFACANAFNNSIIPEDTFFPSPESSCDVKLVEKSFETDTNLNFQNLSVIGFRILLLKV | 2 (aa) |
| | AGFNLLMTLRLWSS | |
| 297 | GACCTGAACAAGGTGTTCCCACCCGAGGTCGCTGTGTTTGAGCCATCAGAAGCAGAGATCTCC | TRBC1 (nt) |
| | CACACCCAAAAGGCCACACTGGTGTGCCTGGCCACAGGCTTCTTCCCCGACCACGTGGAGCTG | |
| | AGCTGGTGGGTGAATGGGAAGGAGGTGCACAGTGGGGTCAGCACAGACCCGCAGCCCCTCAAG | |
| | GAGCAGCCCGCCCTCAATGACTCCAGATACTGCCTGAGCAGCCGCCTGAGGGTCTCGGCCACC | |
| | TTCTGGCAGAACCCCCGCAACCACTTCCGCTGTCAAGTCCAGTTCTACGGGCTCTCGGAGAAT | |
| | GACGAGTGGACCCAGGATAGGGCCAAACCCGTCACCCAGATCGTCAGCGCCGAGGCCTGGGGT | |
| | AGAGCAGACTGTGGCTTTACCTCGGTGTCCTACCAGCAAGGGGTCCTGTCTGCCACCATCCTC | |
| | TATGAGATCCTGCTAGGGAAGGCCACCCTGTATGCTGTGCTGGTCAGCGCCCTTGTGTTGATG | |
| | GCCATGGTCAAGAGAAAGGATTTCTGA | |
| 298 | DLNKVFPPEVAVFEPSEAEISHTQKATLVCLATGFFPDHVELSWWVNGKEVHSGVSTDPQPLK | TRBC1 (aa) |
| | EQPALNDSRYCLSSRLRVSATFWQNPRNHFRCQVQFYGLSENDEWTQDRAKPVTQIVSAEAWG | |
| | RADCGFTSVSYQQGVLSATILYEILLGKATLYAVLVSALVLMAMVKRKDF | |
| 299 | GACCTGAAAAACGTGTTCCCACCCGAGGTCGCTGTGTTTGAGCCATCAGAAGCAGAGATCTCC | TRBC2 (nt) |
| | CACACCCAAAAGGCCACACTGGTATGCCTGGCCACAGGCTTCTACCCCGACCACGTGGAGCTG | |
| | AGCTGGTGGGTGAATGGGAAGGAGGTGCACAGTGGGGTCAGCACAGACCCGCAGCCCCTCAAG | |
| | GAGCAGCCCGCCCTCAATGACTCCAGATACTGCCTGAGCAGCCGCCTGAGGGTCTCGGCCACC | |
| | TTCTGGCAGAACCCCCGCAACCACTTCCGCTGTCAAGTCCAGTTCTACGGGCTCTCGGAGAAT | |
| | GACGAGTGGACCCAGGATAGGGCCAAACCTGTCACCCAGATCGTCAGCGCCGAGGCCTGGGGT | |
| | AGAGCAGACTGTGGCTTCACCTCCGAGTCTTACCAGCAAGGGGTCCTGTCTGCCACCATCCTC | |
| | TATGAGATCTTGCTAGGGAAGGCCACCTTGTATGCCGTGCTGGTCAGTGCCCTCGTGCTGATG | |
| | GCCATGGTCAAGAGAAAGGATTCCAGAGGCTAG | |
| 300 | DLKNVFPPEVAVFEPSEAEISHTQKATLVCLATGFYPDHVELSWWVNGKEVHSGVSTDPQPLK | TRBC2 (aa) |
| | EQPALNDSRYCLSSRLRVSATFWQNPRNHERCQVQFYGLSENDEWTQDRAKPVTQIVSAEAWG | |
| | RADCGFTSESYQQGVLSATILYEILLGKATLYAVLVSALVLMAMVKRKDSRG | |
| 301 | ATGGCCTCTGCTCCCATCTCTATGCTTGCTATGCTTTTTACATTAAGCGGCCTGAGAGCCCAA | TCR A Vα |
| | TCTGTTGCCCAACCTGAAGATCAAGTTAATGTTGCCGAAGGCAATCCTCTGACAGTGAAATGC | (nt) codon |
| | ACCTATTCTGTGTCTGGCAACCCTTATCTTTTTTGGTATGTGCAATACCCCAACAGGGGCCTG | optimized |
| | CAGTTTTTACTGAAATACATCACAGGCGATAACCTGGTGAAGGGCAGCTATGGCTTTGAAGCT | |
| | GAATTCAACAAGAGCCAGACAAGCTTCCACCTGAAGAAACCTTCTGCCCTTGTTTCTGACTCT | |
| | GCTTTGTACTTTTGTGCTGTGAGAGGCGGATCTTACAAATACATCTTTGGAACAGGCACCAGG | |
| | CTGAAGGTTTTAGCTAATATTCAGAACCCTGATCCTGCCGTGTATCAGCTGAGAGACTCTAAA | |
| | AGCTCTGACAAGTCTGTGTGCCTGTTCACCGACTTCGACTCTCAGACAAATGTGTCTCAGTCT | |
| | AAGGACAGCGATGTGTACATCACCGACAAGACAGTGCTTGACATGAGGTCTATGGACTTCAAG | |
| | AGCAACTCTGCTGTGGCTTGGAGCAACAAATCTGACTTTGCTTGTGCTAACGCCTTCAACAAC | |
| | AGCATTATTCCTGAAGACACCTTTTTCCCCAGCCCTGAATCTAGCTGTGATGTGAAGCTTGTG | |
| | GAGAAAAGCTTTGAGACCGACACCAACCTTAACTTCCAGAACCTGTCTGTGATTGGATTCAGG | |
| | ATTCTGCTGCTTAAAGTGGCCGGCTTCAATCTTCTTATGACCCTTAGGCTGTGGAGCAGC | |
| 302 | ATGGAGACACTGCTGGGAGTGTCTTTGGTTATTCTTTGGCTTCAACTGGCTAGGGTTAATAGC | TCR B Vα |
| | CAACAGGGAGAAGAGGATCCTCAAGCTTTGTCTATTCAGGAGGGAGAAAACGCCACCATGAAC | (nt) codon |
| | TGCAGCTACAAGACCTCTATCAACAACCTTCAGTGGTACAGACAGAATTCTGGAAGAGGCCTT | optimized |
| | GTGCACCTGATCTTGATCAGGAGCAATGAAAGAGAGAAGCACAGCGGAAGACTTAGAGTGACC | |
| | CTTGACACCAGCAAAAAAAGCTCTAGCCTGCTGATTACAGCTTCTAGGGCTGCTGATACAGCT | |
| | TCTTATTTCTGTGCTACAGCTCCTGGATCTGGAACCTACAAATACATCTTTGGAACAGGCACC | |
| | AGGCTGAAGGTTTTAGCTAATATTCAGAATCCTGACCCTGCCGTGTACCAACTGAGAGATTCT | |
| | AAAAGCTCTGACAAGAGCGTGTGCCTGTTCACCGACTTCGACTCTCAAACAAATGTGTCTCAG | |
| | TCTAAGGACAGCGACGTGTACATCACCGACAAAACAGTGCTTGACATGAGGTCTATGGACTTC | |
| | AAGAGCAACTCTGCTGTTGCCTGGAGCAACAAGTCTGACTTTGCTTGTGCTAACGCTTTCAAC | |
| | AACAGCATCATCCCTGAAGACACATTCTTTCCCAGCCCTGAATCTAGCTGTGATGTGAAGCTG | |
| | GTGGAGAAAAGCTTTGAGACCGACACCAACCTTAACTTCCAGAACCTTTCTGTGATTGGATTC | |
| | AGGATCCTGCTGCTGAAAGTGGCTGGCTTTAATCTTTTGATGACACTGAGGCTGTGGAGCAGC | |
| 303 | ATGGAGACCCTTTTGGGCCTGCTTATCTTGTGGCTTCAATTACAGTGGGTGAGCTCTAAACAA | TCR C Vα |
| | GAAGTTACCCAAATTCCTGCTGCCCTGTCTGTTCCCGAAGGAGAAAATTTGGTTCTGAACTGT | (nt) codon |
| | AGCTTCACCGATTCTGCTATTTACAACCTGCAATGGTTTAGGCAGGACCCTGGCAAAGGACTG | optimized |
| | ACATCTCTTTTGTTGATCCAGTCTTCTCAGAGAGAGCAAACATCTGGAAGACTTAATGCCAGC | |
| | CTGGACAAGTCTTCTGGAAGGTCTACATTATACATTGCCGCTTCTCAACCTGGAGATTCTGCT | |
| | ACATATTTGTGTGCTGTTAGGCCTAGGACCTCTGGAACATATAAATACATCTTTGGCACAGGC | |
| | ACCAGGCTGAAGGTGTTAGCTAATATCCAAAATCCTGATCCTGCCGTGTACCAGCTGAGAGAC | |
| | TCTAAAAGCTCTGATAAGAGCGTTTGCCTGTTCACCGACTTCGACAGCCAGACAAATGTGTCT | |
| | CAATCTAAGGACAGCGATGTGTACATCACCGACAAGACAGTGCTTGACATGAGGTCTATGGAC | |
| | TTCAAGAGCAACTCTGCTGTTGCCTGGAGCAACAAGTCTGACTTTGCTTGTGCTAACGCTTTC | |
| | AACAACAGCATCATTCCTGAAGATACCTTCTTCCCCAGCCCTGAATCTAGCTGTGATGTGAAA | |
| | CTGGTGGAGAAAAGCTTTGAGACCGACACCAACCTGAACTTCCAAAACCTGTCTGTGATTGGC | |
| | TTCAGGATCCTGTTACTGAAAGTGGCTGGCTTCAATCTTTTAATGACCCTGAGGCTGTGGAGC | |
| | AGC | |
| 304 | ATGGCCTCTGCCCCTATTAGCATGCTTGCTATGCTTTTTACATTGTCTGGCCTGAGAGCCCAA | TCR D Vα |
| | TCTGTTGCTCAGCCTGAAGATCAAGTTAATGTTGCCGAAGGCAATCCTCTGACAGTGAAATGC | (nt) codon |
| | ACCTATTCTGTGTCTGGCAACCCCTATCTTTTTTGGTATGTTCAATACCCCAACAGGGGCCTG | optimized |
| | CAGTTCTTGCTGAAATACATTACAGGCGATAATCTGGTGAAGGGCAGCTATGGCTTTGAAGCT | |
| | GAATTTAACAAGAGCCAGACCAGCTTCCACCTTAAGAAACCTTCTGCCCTTGTTTCTGATTCT | |
| | GCTTTGTACTTCTGTGCTGTGAGAGCTCCTACCTCTGGAACCTACAAGTACATCTTTGGAACA | |
| | GGCACCAGACTGAAGGTGTTAGCTAATATTCAGAATCCTGACCCTGCCGTTTACCAGCTGAGA | |
| | GACTCTAAAAGCTCTGACAAAAGCGTGTGCCTTTTCACCGACTTCGACAGCCAGACAAATGTG | |
| | TCTCAATCTAAGGACAGCGATGTGTACATCACCGACAAGACAGTGCTTGACATGAGGTCTATG | |
| | GATTTCAAGAGCAACTCTGCTGTGGCCTGGAGCAATAAATCTGACTTTGCTTGTGCTAACGCC | |
| | TTCAACAATAGCATTATTCCTGAAGACACCTTCTTCCCCAGCCCTGAATCTAGCTGTGATGTT | |
| | AAACTGGTGGAGAAGAGCTTTGAGACCGACACCAACCTGAACTTCCAGAACTTGTCTGTGATT | |
| | GGCTTTAGGATCTTGCTGCTGAAAGTTGCTGGCTTTAATCTTCTGATGACCCTGAGGCTGTGG | |
| | AGCAGC | |
| 305 | ATGGCCTCTGCTCCTATCAGCATGTTGGCCATGTTGTTTACATTGTCTGGACTGAGAGCTCAA | TCR E Vα |
| | TCTGTTGCTCAACCCGAAGATCAGGTGAATGTTGCTGAAGGCAATCCTCTGACAGTGAAGTGC | (nt) codon |
| | ACCTATTCTGTGTCTGGAAACCCCTACCTTTTTTGGTATGTTCAATACCCCAACAGGGGACTG | optimized |
| | CAGTTCCTTCTGAAATACATTACAGGCGATAACCTGGTGAAGGGCAGCTACGGCTTTGAAGCT | |
| | GAATTCAATAAGAGCCAAACCAGCTTCCATCTGAAAAAACCTTCTGCCCTTGTGTCTGATAGC | |
| | GCTCTGTATTTTTGTGCTGTTAGAGGCCCTACATCTGGCACATACAAATACATCTTTGGAACC | |
| | GGAACCAGGCTGAAGGTGTTAGCTAATATTCAGAACCCTGATCCTGCTGTGTACCAGCTGAGA | |
| | GACTCTAAAAGCTCTGACAAGTCTGTGTGCCTTTTCACCGACTTCGACAGCCAGACAAATGTG | |
| | TCTCAGTCTAAGGACTCTGATGTGTACATCACCGACAAGACAGTGTTAGACATGAGGTCTATG | |
| | GACTTCAAGAGCAACTCTGCTGTGGCCTGGTCTAACAAGTCTGATTTTGCTTGTGCTAACGCC | |
| | TTCAACAACAGCATTATTCCTGAAGACACCTTCTTCCCTAGCCCTGAATCTAGCTGTGATGTG | |
| | AAGTTGGTGGAGAAAAGCTTCGAGACCGACACCAACCTTAACTTCCAAAACCTGTCTGTGATT | |
| | GGCTTCAGGATCCTGCTTCTGAAAGTGGCTGGATTTAATCTGCTGATGACACTGAGGCTGTGG | |
| | AGCAGC | |
| 306 | ATGACCAGCATCAGGGCCGTGTTCATTTTCCTGTGGTTACAACTGGATCTTGTGAATGGCGAG | TCR F Vα |
| | AATGTTGAACAACATCCTTCTACACTTTCTGTGCAGGAGGGAGACTCTGCCGTGATTAAGTGT | (nt) codon |
| | ACATATTCTGACTCTGCCAGCAACTACTTCCCCTGGTACAAGCAGGAACTTGGAAAAAGACCT | optimized |
| | CAGCTGATCATCGACATCAGGAGCAATGTGGGCGAAAAGAAAGACCAAAGGATCGCTGTGACC | |
| | TTGAACAAGACAGCCAAACATTTCAGCCTGCACATCACAGAGACCCAACCTGAAGATTCTGCC | |
| | GTGTATTTCTGTGCTGCTCATTTGACAGGAGGAGGCAACAAATTGACCTTTGGCACAGGAACA | |
| | CAGCTTAAAGTGGAACTGAACATTCAGAACCCTGACCCTGCCGTGTACCAATTGAGAGACTCT | |
| | AAAAGCTCTGACAAGAGCGTGTGCCTGTTCACCGACTTTGACTCTCAGACAAATGTGTCTCAG | |
| | TCTAAGGACAGCGACGTGTACATCACCGACAAGACAGTTCTTGACATGAGGTCTATGGACTTC | |
| | AAGAGCAATTCTGCTGTGGCCTGGAGCAACAAATCTGACTTTGCTTGTGCTAACGCTTTCAAC | |
| | AACAGCATCATCCCTGAAGACACCTTTTTCCCTAGCCCTGAATCTAGCTGTGATGTGAAGCTG | |
| | GTGGAGAAAAGCTTTGAGACCGACACCAACCTTAACTTCCAAAACCTGTCTGTGATTGGCTTC | |
| | AGGATCCTGCTGCTTAAAGTTGCCGGCTTTAACCTGTTGATGACCTTAAGGCTGTGGAGCAGC | |
| 307 | ATGGAGACACTGCTGGGAGTGTCTCTTGTGATTTTATGGCTTCAACTGGCTAGGGTGAACTCT | TCR G Vα |
| | CAACAAGGAGAAGAAGATCCTCAAGCCTTGAGCATCCAAGAAGGAGAAAATGCCACCATGAAC | (nt) codon |
| | TGCAGCTACAAGACCAGCATCAACAACCTTCAGTGGTACAGACAGAATTCTGGAAGAGGCCTT | optimized |
| | GTGCACCTGATCTTGATCAGGAGCAATGAAAGAGAGAAGCACAGCGGAAGACTGAGAGTGACA | |
| | CTTGACACCAGCAAAAAGAGCTCTAGCTTGCTGATTACAGCTAGCAGAGCTGCTGACACAGCT | |
| | TCTTATTTTTGTGCTGGAACAGGAGCTAATAACCTGTTCTTTGGCACAGGCACAAGACTGACC | |
| | GTTATTCCCTACATTCAAAATCCCGATCCTGCCGTGTACCAACTGAGAGACTCTAAAAGCTCT | |
| | GACAAAAGCGTGTGCCTGTTCACCGACTTCGACTCTCAGACAAATGTGTCTCAATCTAAGGAC | |
| | AGCGACGTGTACATCACCGACAAGACAGTTCTTGACATGAGGTCTATGGACTTCAAGTCTAAC | |
| | TCTGCTGTGGCCTGGTCTAACAAGTCTGATTTTGCTTGTGCTAACGCCTTCAACAACAGCATT | |
| | ATTCCTGAAGATACCTTCTTCCCCAGCCCTGAATCTAGCTGTGATGTGAAACTGGTGGAGAAA | |
| | AGCTTTGAGACCGACACCAACCTGAACTTCCAAAATCTGTCTGTGATTGGCTTCAGGATCCTG | |
| | CTTCTGAAAGTGGCCGGCTTTAATTTGCTTATGACCCTGAGGCTGTGGTCTAGC | |
| 308 | ATGACCAGCATCAGGGCCGTTTTTATTTTCCTTTGGCTGCAACTGGACCTTGTGAACGGAGAG | TCR H Vα |
| | AATGTTGAGCAACATCCTTCTACACTGTCTGTGCAGGAAGGAGATAGCGCTGTGATCAAGTGT | (nt) codon |
| | ACCTATTCTGACTCTGCCAGCAACTACTTCCCTTGGTACAAGCAGGAGCTTGGAAAAAGACCT | optimized |
| | CAGCTGATCATCGACATCAGGTCTAATGTGGGCGAGAAGAAAGACCAAAGGATCGCTGTGACA | |
| | TTGAACAAGACAGCTAAGCACTTCAGCCTGCATATCACAGAGACCCAACCTGAAGATTCTGCT | |
| | GTTTATTTCTGTGCTGCTCCTTTAGCTGGAGGAGGAGCTGATGGATTAACCTTTGGAAAAGGA | |
| | ACACATCTGATCATCCAACCCTATATCCAAAACCCTGACCCTGCCGTGTACCAGTTAAGAGAC | |
| | TCTAAAAGCTCTGACAAGAGCGTGTGCCTGTTCACCGACTTCGATTCTCAGACAAATGTGTCT | |
| | CAGTCTAAGGACAGCGACGTGTACATCACCGACAAAACAGTGCTTGACATGAGGTCTATGGAC | |
| | TTCAAAAGCAACTCTGCTGTGGCCTGGAGCAACAAGTCTGATTTTGCTTGTGCTAACGCTTTC | |
| | AACAACAGCATCATTCCTGAAGATACCTTCTTCCCCAGCCCTGAATCTAGCTGTGATGTTAAG | |
| | CTGGTGGAGAAAAGCTTTGAGACCGACACCAACCTGAACTTCCAGAACTTGTCTGTGATTGGA | |
| | TTCAGGATCCTGCTGCTGAAAGTTGCCGGCTTTAATTTGTTAATGACCCTGAGGCTGTGGAGC | |
| | AGC | |
| 309 | ATGGAGACCCTGCTGGGCCTGTTAATCTTATGGTTGCAATTGCAATGGGTGTCTAGCAAACAG | TCR I Vα (nt) |
| | GAGGTTACACAGATTCCTGCCGCTTTATCTGTGCCTGAAGGAGAAAACTTGGTTCTGAACTGC | codon |
| | AGCTTCACCGATTCTGCCATTTACAATCTGCAATGGTTTAGGCAGGACCCTGGCAAAGGACTG | optimized |
| | ACATCTCTGTTGTTGATTCAGTCTTCTCAGAGAGAGCAGACATCTGGAAGACTTAATGCCAGC | |
| | CTGGACAAATCTTCTGGAAGGAGCACACTTTATATCGCTGCTTCTCAACCTGGAGATTCTGCT | |
| | ACCTATCTTTGTGCTGTTAGGGGCACAACATCTGGAACCTACAAATACATCTTTGGAACAGGC | |
| | ACCAGGCTGAAGGTGTTAGCTAATATTCAGAACCCTGATCCTGCTGTGTACCAACTGAGAGAC | |
| | TCTAAGAGCTCTGACAAGAGCGTTTGCCTTTTCACCGACTTCGACTCTCAGACAAACGTGTCT | |
| | CAGTCTAAGGACTCTGATGTGTACATCACCGACAAGACCGTGTTGGACATGAGGTCTATGGAC | |
| | TTTAAGAGCAATTCTGCTGTGGCCTGGAGCAACAAGTCTGATTTTGCTTGTGCTAACGCCTTT | |
| | AACAACAGCATCATTCCTGAAGACACCTTTTTCCCCAGCCCTGAATCTAGCTGTGATGTGAAA | |
| | CTGGTGGAGAAATCTTTCGAGACCGACACCAACCTGAACTTTCAAAACCTGTCTGTGATTGGC | |
| | TTCAGGATCCTGCTGCTTAAAGTTGCCGGCTTTAATCTGTTAATGACCCTGAGGCTGTGGAGC | |
| | AGC | |
| 310 | ATGATGAAGAGCCTGAGAGTGCTGCTGGTTATTCTGTGGCTTCAATTAAGCTGGGTTTGGAGC | TCR J Vα (nt) |
| | CAGCAAAAGGAAGTTGAACAAGATCCTGGACCTCTTTCTGTTCCTGAGGGAGCCATTGTTTCT | codon |
| | CTGAATTGCACCTACAGCAACTCTGCCTTCCAGTACTTCATGTGGTACAGACAGTACAGCAGA | optimized |
| | AAAGGCCCTGAGTTGCTGATGTACACATACAGCTCTGGCAACAAAGAAGATGGCAGGTTTACA | |
| | GCTCAGGTGGACAAAAGCAGCAAGTACATCAGCTTGTTCATCAGAGACTCTCAGCCCTCTGAT | |
| | TCTGCCACATATCTTTGTGCTATGAGGGCTACATCTGGCACCTACAAGTATATTTTTGGCACA | |
| | GGAACCAGGCTGAAGGTGTTAGCTAATATCCAAAATCCTGACCCTGCCGTTTACCAGCTGAGA | |
| | GATTCTAAAAGCTCTGACAAGAGCGTGTGCCTTTTCACCGACTTCGACAGCCAAACAAATGTG | |
| | TCTCAGTCTAAGGACAGCGATGTGTACATCACCGACAAGACAGTGCTTGACATGAGGTCTATG | |
| | GACTTCAAGAGCAACTCTGCTGTGGCTTGGAGCAACAAGTCTGACTTTGCTTGTGCTAACGCT | |
| | TTCAACAACAGCATTATTCCTGAAGACACCTTCTTCCCCAGCCCTGAATCTTCTTGTGATGTG | |
| | AAGTTGGTGGAGAAGAGCTTTGAGACCGACACCAACCTGAACTTTCAGAACCTGTCTGTGATT | |
| | GGCTTTAGGATTCTGCTGCTGAAAGTGGCTGGCTTTAATTTGCTTATGACCCTGAGGCTGTGG | |
| | AGCAGC | |
| 311 | ATGAAGCTGGTGACCAGCATCACCGTTCTGCTTTCTTTGGGAATTATGGGAGATGCCAAGACC | TCR K Vα |
| | ACACAGCCTAACTCTATGGAGTCTAACGAAGAAGAGCCTGTGCACTTGCCTTGTAACCACAGC | (nt) codon |
| | ACAATCTCTGGAACAGACTATATTCATTGGTACAGGCAGCTTCCCAGCCAAGGACCTGAGTAT | optimized |
| | GTTATTCATGGACTTACAAGCAACGTGAACAACAGAATGGCCTCTCTGGCTATCGCTGAAGAT | |
| | AGAAAGAGCTCTACATTGATCCTGCACAGAGCTACACTTAGGGATGCTGCTGTTTATTATTGC | |
| | ATCCTGAGAGCCGGCTCTGGAACCTATAAATATATTTTCGGAACAGGCACCAGGCTGAAGGTT | |
| | TTAGCTAATATTCAGAACCCTGATCCTGCCGTTTATCAGTTGAGGGACAGCAAAAGCTCTGAC | |
| | AAGTCTGTGTGCCTTTTCACCGACTTTGACAGCCAGACCAATGTGTCTCAGTCTAAGGACTCT | |
| | GATGTGTACATCACCGACAAGACAGTGCTTGACATGAGGTCTATGGACTTCAAGAGCAACTCT | |
| | GCTGTGGCTTGGAGCAACAAATCTGACTTTGCTTGTGCTAACGCCTTCAACAACAGCATTATT | |
| | CCTGAAGACACCTTCTTTCCCAGCCCTGAATCTAGCTGTGATGTGAAACTGGTGGAGAAGTCT | |
| | TTCGAGACCGACACCAACCTTAACTTCCAGAACCTGTCTGTGATTGGATTCAGGATCCTGCTG | |
| | CTTAAAGTTGCCGGCTTTAATCTGCTGATGACCCTGAGACTGTGGAGCAGC | |
| 312 | ATGGAGACCCTTTTGGGACTGCTTATCCTTTGGTTGCAGCTTCAATGGGTTAGCAGCAAGCAA | TCR L Vα |
| | GAGGTTACCCAAATTCCTGCTGCCTTATCTGTGCCTGAAGGAGAAAACTTGGTTCTGAACTGC | (nt) codon |
| | AGCTTCACAGATAGCGCCATCTATAACTTACAGTGGTTTAGGCAGGACCCTGGCAAAGGACTT | optimized |
| | ACCTCTCTGTTGTTGATTCAGTCTTCTCAGAGAGAGCAGACATCTGGAAGACTTAATGCCAGC | |
| | CTGGACAAATCTTCTGGAAGGAGCACATTGTACATTGCTGCTAGCCAACCTGGAGATTCTGCT | |
| | ACCTATCTGTGTGCTGTTACAAGGACAGGAGCCAACAACCTGTTTTTTGGCACAGGAACAAGA | |
| | CTGACCGTTATTCCCTATATCCAAAACCCTGACCCTGCCGTGTATCAGCTTAGAGACTCTAAA | |
| | AGCTCTGACAAGAGCGTGTGCCTGTTCACCGACTTCGATTCTCAGACAAATGTGTCTCAGTCT | |
| | AAGGACAGCGACGTGTACATCACCGACAAAACAGTGCTTGACATGAGGTCTATGGACTTCAAG | |
| | AGCAATTCTGCTGTGGCCTGGAGCAACAAATCTGACTTTGCTTGTGCTAATGCCTTCAACAAC | |
| | AGCATCATTCCTGAAGACACCTTCTTCCCTAGCCCTGAATCTTCTTGTGATGTGAAGCTGGTG | |
| | GAGAAGAGCTTTGAGACCGACACCAACCTTAACTTCCAAAACCTGAGCGTGATCGGATTTAGG | |
| | ATCCTGCTGCTGAAAGTTGCTGGCTTTAATCTGCTGATGACCCTTAGGCTGTGGAGCAGC | |
| 313 | ATGGAGACACTTCTTGGCCTTCTTATTTTGTGGCTTCAGCTGCAATGGGTGAGCAGCAAACAG | TCR M Vα |
| | GAAGTTACACAAATTCCTGCTGCTCTGTCTGTGCCTGAAGGAGAAAACTTGGTTCTGAACTGT | (nt) codon |
| | AGCTTCACCGATAGCGCCATTTACAATCTGCAATGGTTTAGGCAAGACCCTGGCAAAGGACTG | optimized |
| | ACCTCTCTTTTGCTGATTCAGTCTTCTCAGAGAGAGCAGACATCTGGAAGACTTAATGCCAGC | |
| | CTGGACAAATCTTCTGGAAGGAGCACCTTATACATTGCTGCTTCTCAACCCGGAGATTCTGCT | |
| | ACCTATCTTTGTGCTGTTAGAGGATCTACCTCTGGCACCTACAAATATATCTTCGGAACAGGC | |
| | ACCAGGCTGAAGGTTTTAGCTAATATCCAAAATCCTGACCCTGCCGTGTACCAGCTGAGAGAT | |
| | TCTAAAAGCTCTGACAAAAGCGTGTGCCTGTTCACCGACTTCGACAGCCAAACAAATGTGTCT | |
| | CAATCTAAGGACAGCGACGTGTACATCACCGACAAAACAGTGCTTGACATGAGGTCTATGGAC | |
| | TTCAAAAGCAACTCTGCTGTGGCCTGGAGCAACAAATCTGACTTTGCTTGTGCTAATGCCTTC | |
| | AACAACAGCATTATTCCTGAAGACACCTTCTTCCCCAGCCCTGAATCTAGCTGTGATGTTAAG | |
| | CTGGTTGAGAAGAGCTTTGAGACCGACACCAACCTGAACTTCCAGAACTTATCTGTGATTGGC | |
| | TTCAGGATCCTGCTTCTGAAAGTGGCCGGATTTAATTTGCTGATGACCCTGAGGCTTTGGAGC | |
| | AGC | |
| 314 | ATGACCAGCATCAGGGCCGTTTTTATTTTCCTTTGGTTGCAGCTGGACTTGGTGAACGGAGAA | TCR N Vα |
| | AATGTTGAACAGCATCCTTCTACCCTGTCTGTGCAAGAAGGAGACTCTGCTGTGATCAAGTGT | (nt) codon |
| | ACCTACTCTGACTCTGCCAGCAACTACTTCCCTTGGTACAAGCAAGAGCTTGGAAAGAGACCT | optimized |
| | CAGCTGATCATCGACATTAGGTCTAATGTGGGCGAGAAGAAGGACCAAAGGATCGCTGTTACA | |
| | TTGAACAAGACAGCCAAACATTTTAGCCTGCACATTACAGAGACCCAACCTGAAGACAGCGCT | |
| | GTTTACTTTTGTGCTGCTATTGTGGGCAACCAGTTCTATTTTGGCACAGGCACATCTTTGACC | |
| | GTGATTCCTAATATCCAAAACCCTGACCCTGCCGTGTACCAACTGAGAGACTCTAAATCTTCT | |
| | GACAAGAGCGTGTGCCTGTTCACCGACTTCGACTCTCAAACAAATGTGTCTCAGTCTAAGGAC | |
| | AGCGACGTGTACATCACCGACAAAACAGTGCTTGACATGAGGTCTATGGACTTCAAGAGCAAC | |
| | TCTGCTGTGGCTTGGAGCAACAAGTCTGACTTTGCTTGTGCTAATGCCTTCAACAACAGCATC | |
| | ATCCCTGAAGACACCTTCTTTCCTAGCCCTGAATCTAGCTGTGATGTGAAGCTGGTGGAGAAG | |
| | AGCTTTGAGACAGACACCAACCTTAACTTCCAGAACCTGAGCGTGATTGGCTTTAGGATCCTG | |
| | TTACTGAAAGTTGCCGGCTTTAACCTGCTGATGACCCTTAGGTTATGGAGCAGC | |
| 315 | ATGACCAGCATCAGGGCTGTTTTTATTTTCCTGTGGCTGCAGCTGGATCTTGTGAATGGAGAG | TCR O Vα |
| | AATGTGGAACAACATCCTTCTACCTTAAGCGTGCAAGAGGGAGATAGCGCTGTGATCAAGTGT | (nt) codon |
| | ACCTATTCTGACTCTGCCAGCAACTACTTCCCTTGGTACAAGCAGGAGCTTGGAAAAAGACCT | optimized |
| | CAGCTGATCATCGACATCAGGTCTAATGTGGGCGAGAAGAAAGACCAAAGGATCGCTGTGACA | |
| | TTGAACAAGACAGCCAAACATTTCAGCCTGCACATCACAGAGACCCAACCTGAAGATTCTGCT | |
| | GTGTACTTCTGTGCTATGAGAAATAACAATGACATGAGGTTCGGAGCCGGCACCAGACTGACA | |
| | GTTAAACCTAATATCCAAAACCCTGATCCTGCCGTGTACCAGCTGAGAGACTCTAAATCTTCT | |
| | GACAAGTCTGTGTGCCTGTTCACCGACTTCGACAGCCAAACAAATGTGTCTCAGTCTAAGGAC | |
| | AGCGATGTGTACATCACCGACAAGACAGTGCTTGACATGAGGTCTATGGACTTCAAGAGCAAC | |
| | TCTGCTGTTGCCTGGAGCAACAAATCTGACTTTGCTTGCGCTAACGCTTTCAACAACAGCATC | |
| | ATCCCTGAAGATACCTTCTTCCCTAGCCCTGAATCTAGCTGTGATGTGAAGCTGGTGGAGAAA | |
| | AGCTTTGAGACCGACACCAACCTTAACTTCCAGAACCTGTCTGTGATTGGCTTCAGAATCCTG | |
| | CTGCTGAAAGTTGCTGGCTTCAATCTGCTGATGACCTTAAGGCTGTGGAGCAGC | |
| 316 | ATGCTGCTGCTGCTGATCCCTGTGCTGGGAATGATTTTTGCCTTAAGAGATGCCAGAGCTCAG | TCR P Vα |
| | TCTGTGAGCCAGCATAATCACCATGTGATTCTGTCTGAAGCTGCTTCTCTGGAGTTGGGATGC | (nt) codon |
| | AACTATAGCTATGGAGGCACAGTGAATCTGTTCTGGTATGTGCAGTATCCTGGACAACACCTG | optimized |
| | CAGCTGCTGTTAAAGTACTTCTCTGGAGATCCTCTGGTTAAAGGCATCAAGGGCTTTGAGGCT | |
| | GAGTTCATCAAGAGCAAGTTCAGCTTCAACCTGAGAAAGCCCTCTGTGCAATGGTCTGATACA | |
| | GCTGAGTATTTCTGTGCTGTTAGGGATTCTGGCGCTGGATCTTACCAACTGACATTCGGAAAA | |
| | GGCACCAAACTTAGCGTGATCCCTAATATCCAAAATCCTGACCCTGCTGTGTACCAGCTGAGA | |
| | GACTCTAAAAGCTCTGACAAAAGCGTGTGCCTTTTCACCGACTTCGACAGCCAGACAAATGTG | |
| | TCTCAATCTAAGGACAGCGATGTGTACATCACCGACAAGACAGTGCTTGACATGAGGTCTATG | |
| | GACTTCAAGAGCAACTCTGCTGTTGCCTGGAGCAACAAATCTGATTTTGCTTGCGCTAACGCC | |
| | TTCAACAACAGCATCATTCCTGAAGACACCTTCTTCCCTTCTCCTGAGTCTTCTTGTGATGTG | |
| | AAGCTGGTGGAGAAGAGCTTTGAGACCGACACCAACCTTAACTTCCAAAACCTGTCTGTGATT | |
| | GGCTTCAGGATCCTTTTGCTGAAAGTGGCCGGATTTAATCTGCTTATGACACTTAGGCTGTGG | |
| | AGCAGC | |
| 317 | ATGACCAGCATCAGGGCCGTGTTTATTTTTCTGTGGCTGCAGTTAGACTTGGTGAATGGAGAA | TCR Q Vα |
| | AATGTGGAGCAGCATCCCTCTACCTTATCTGTTCAGGAAGGAGATTCTGCTGTGATCAAGTGC | (nt) codon |
| | ACCTATTCTGACTCTGCCAGCAACTACTTCCCTTGGTACAAGCAAGAGCTGGGAAAAAGACCT | optimized |
| | CAGCTGATCATCGACATTAGGTCTAATGTGGGCGAGAAGAAGGACCAAAGGATCGCTGTTACA | |
| | TTGAACAAGACAGCTAAACATTTCAGCCTGCACATTACAGAGACCCAACCTGAAGACAGCGCT | |
| | GTGTACTTTTGTGCTGCTTCTAATCCTACAGGAGCCAACAGCAAGCTGACCTTTGGAAAAGGC | |
| | ATTACACTGTCTGTTAGACCTGATATTCAGAACCCTGACCCTGCCGTTTATCAGCTGAGAGAC | |
| | TCTAAAAGCTCTGACAAGAGCGTGTGCCTTTTCACCGACTTCGACTCTCAGACAAATGTGTCT | |
| | CAGTCTAAGGACAGCGATGTGTACATCACCGACAAGACAGTGCTTGACATGAGGTCTATGGAT | |
| | TTCAAGAGCAACTCTGCTGTGGCCTGGAGCAACAAATCTGACTTTGCTTGTGCTAATGCCTTC | |
| | AACAACAGCATCATTCCTGAAGACACCTTCTTCCCTAGCCCTGAATCTAGCTGTGATGTTAAG | |
| | CTGGTGGAGAAGAGCTTTGAGACCGACACCAACCTTAACTTCCAAAACCTTTCTGTGATTGGC | |
| | TTTAGAATCCTGCTGCTGAAAGTGGCCGGATTTAATCTGCTTATGACCCTTAGGCTGTGGAGC | |
| | AGC | |
| 318 | ATGGAGACCCTGCTGGGCGTGTCTTTGGTTATTCTTTGGCTGCAATTAGCTAGAGTGAACTCT | TCR R Vα |
| | CAACAAGGAGAAGAGGATCCTCAGGCTTTGTCTATCCAAGAGGGAGAAAATGCCACCATGAAC | (nt) codon |
| | TGCAGCTACAAGACCAGCATCAACAACCTTCAGTGGTACAGACAGAATTCTGGAAGAGGCCTT | optimized |
| | GTGCACCTGATCTTGATCAGGAGCAATGAAAGAGAGAAGCACAGCGGAAGACTGAGAGTGACA | |
| | CTTGACACCTCTAAGAAATCTTCTAGCTTGTTGATTACCGCTAGCAGGGCCGCTGATACAGCT | |
| | TCTTACTTTTGTGCTACAGAAATGAACAGCAACTACCAGTTAATCTGGGGCGCTGGCACAAAG | |
| | CTGATCATTAAGCCTGATATTCAGAATCCTGATCCTGCCGTGTACCAGCTGAGAGACTCTAAG | |
| | TCTTCTGATAAGTCTGTGTGCCTGTTCACCGACTTCGACAGCCAGACAAATGTGTCTCAATCT | |
| | AAGGACAGCGATGTGTACATCACCGACAAGACAGTGCTTGACATGAGGTCTATGGACTTCAAG | |
| | AGCAACTCTGCTGTTGCCTGGAGCAACAAATCTGACTTTGCTTGTGCTAACGCCTTCAATAAC | |
| | AGCATTATTCCCGAAGATACCTTCTTCCCCAGCCCTGAATCTAGCTGTGATGTGAAACTGGTG | |
| | GAGAAAAGCTTTGAGACCGACACCAACCTGAACTTCCAGAACCTGTCTGTTATTGGCTTCAGG | |
| | ATCCTGCTGCTTAAAGTTGCCGGCTTTAATCTGCTTATGACCCTTAGGCTGTGGAGCAGC | |
| 319 | ATGAGGCTGGTGGCCAGAGTGACAGTGTTTCTGACCTTTGGAACAATCATTGACGCTAAAACA | TCR S Vα |
| | ACACAACCCCCCTCTATGGATTGTGCTGAAGGAAGAGCTGCCAATTTACCTTGCAACCACTCT | (nt) codon |
| | ACCATCTCTGGCAATGAGTATGTGTATTGGTATAGGCAGATTCACAGCCAGGGCCCTCAGTAC | optimized |
| | ATCATTCATGGACTTAAGAACAACGAGACCAACGAGATGGCCAGCCTGATTATCACAGAAGAT | |
| | AGGAAGAGCTCTACCTTGATCTTACCTCACGCCACCCTTAGAGATACAGCTGTGTACTACTGC | |
| | ATTGTTAGAGCCAGCACCTCTGGAACCTACAAATACATCTTTGGAACAGGCACAAGGCTGAAG | |
| | GTGTTAGCTAATATCCAGAATCCTGATCCTGCCGTGTATCAGCTGAGAGACTCTAAAAGCTCT | |
| | GACAAGAGCGTTTGCCTGTTCACCGACTTCGACTCTCAGACAAATGTGTCTCAGTCTAAGGAC | |
| | AGCGATGTGTACATCACCGACAAGACAGTGCTTGACATGAGGTCTATGGACTTCAAGAGCAAC | |
| | TCTGCTGTTGCCTGGAGCAACAAATCTGACTTTGCCTGTGCTAATGCCTTCAACAACAGCATC | |
| | ATCCCTGAAGACACCTTTTTTCCCAGCCCTGAATCTAGCTGTGATGTGAAGCTGGTGGAGAAA | |
| | AGCTTTGAGACCGACACCAACCTTAACTTCCAGAACCTTTCTGTTATTGGATTCAGGATCCTG | |
| | CTGCTGAAAGTGGCCGGCTTTAATCTGTTGATGACACTTAGGCTGTGGAGCAGC | |
| 320 | ATGGAGACACTGCTGGGAGTGTCTTTGGTGATTCTTTGGCTTCAACTTGCTAGGGTGAACTCT | TCR T Vα |
| | CAACAAGGAGAAGAGGATCCTCAAGCCTTGAGCATCCAAGAAGGAGAAAATGCCACCATGAAC | (nt) codon |
| | TGCAGCTACAAGACCAGCATCAACAACCTTCAGTGGTACAGACAGAATTCTGGAAGAGGCCTT | optimized |
| | GTGCACCTGATCTTGATCAGGAGCAATGAAAGAGAGAAGCACAGCGGAAGACTTAGAGTGACC | |
| | TTGGACACCAGCAAAAAAAGCTCTAGCCTGCTGATTACAGCTAGCAGAGCTGCTGACACAGCT | |
| | TCTTATTTTTGTGCTACCCCTTCTGGCAATACACCTCTTGTGTTTGGAAAGGGCACAAGACTT | |
| | TCTGTGATTGCTAATATCCAGAACCCTGATCCTGCCGTGTATCAGCTGAGAGATTCTAAAAGC | |
| | TCTGACAAGAGCGTGTGCCTGTTCACCGACTTCGACTCTCAAACAAATGTGTCTCAGTCTAAG | |
| | GACAGCGACGTGTACATCACCGACAAAACAGTGCTTGACATGAGGTCTATGGACTTCAAAAGC | |
| | AACTCTGCTGTGGCCTGGTCTAACAAATCTGACTTTGCTTGTGCTAACGCCTTCAACAACAGC | |
| | ATCATTCCTGAAGATACCTTTTTCCCCAGCCCTGAATCTAGCTGTGATGTGAAGCTGGTGGAA | |
| | AAAAGCTTTGAGACCGACACCAACCTGAACTTCCAGAACCTGTCTGTGATTGGATTCAGGATC | |
| | CTTCTGCTGAAAGTGGCTGGCTTTAATCTGCTGATGACCCTTAGACTGTGGAGCAGC | |
| 321 | ATGGGCACAAGCCTGCTTTGCTGGATGGCCTTATGTTTATTAGGAGCTGATCACGCTGATACA | TCR A Vβ |
| | GGAGTTTCTCAGGATCCTAGGCACAAGATCACCAAAAGGGGCCAGAATGTGACATTCAGGTGT | (nt) codon |
| | GATCCTATTAGCGAACACAATAGGCTGTATTGGTACAGGCAGACATTAGGCCAAGGACCTGAG | optimized |
| | TTTCTGACATACTTCCAGAACGAGGCTCAACTTGAAAAGTCTAGGCTGCTTTCTGATAGGTTC | |
| | AGCGCTGAGAGGCCTAAAGGATCTTTCTCTACCTTGGAGATTCAGAGGACAGAACAAGGCGAT | |
| | TCTGCTATGTATCTTTGTGCTAGCACCCCTGGAACCGTTTATAATGAGCAATTTTTCGGACCT | |
| | GGCACAAGGCTGACCGTGCTTGAAGATCTGAAGAATGTTTTTCCTCCTGAAGTGGCCGTTTTT | |
| | GAGCCTTCTGAAGCTGAGATTAGCCATACACAGAAAGCTACACTGGTGTGTCTGGCTACAGGC | |
| | TTTTACCCTGATCATGTTGAACTGTCTTGGTGGGTTAATGGAAAGGAAGTTCATTCTGGAGTG | |
| | AGCACAGATCCCCAACCCCTTAAAGAACAACCTGCTCTGAATGATAGCAGGTACTGTCTGAGC | |
| | TCTAGGCTTAGGGTTTCTGCCACATTTTGGCAAAATCCCAGGAACCACTTTAGGTGCCAAGTG | |
| | CAGTTTTACGGCCTTAGCGAAAATGACGAATGGACCCAAGACAGAGCTAAACCTGTGACCCAA | |
| | ATTGTTTCTGCTGAAGCTTGGGGAAGAGCTGATTGTGGCTTCACAAGCGAATCTTACCAACAA | |
| | GGCGTTCTGTCTGCCACCATTCTGTACGAGATCTTGTTAGGCAAAGCTACACTGTATGCTGTT | |
| | CTGGTTTCTGCCTTGGTTCTGATGGCCATGGTGAAGAGAAAGGATAGCAGAGGC | |
| 322 | ATGGGCACCAGCTTACTGTGTTGGATGGCCTTATGTTTATTGGGAGCTGATCATGCTGATACA | TCR B Vβ |
| | GGAGTGAGCCAGGACCCTAGACATAAAATCACCAAAAGGGGCCAGAATGTGACCTTCAGGTGT | (nt) codon |
| | GATCCTATTTCTGAACATAACAGGCTGTATTGGTACAGGCAAACCTTAGGACAGGGCCCTGAG | optimized |
| | TTTTTGACATACTTCCAGAACGAAGCTCAGCTTGAGAAGAGCAGGTTACTGTCTGATAGATTC | |
| | TCTGCTGAGAGGCCTAAGGGCTCTTTTAGCACCTTAGAGATCCAAAGGACAGAACAAGGAGAT | |
| | TCTGCTATGTATCTTTGTGCTGCCCCCCCTGATACCTATAATTCTCCTCTGCATTTTGGCAAT | |
| | GGAACAAGGCTGACCGTTACAGAGGATCTGAACAAGGTGTTTCCTCCTGAAGTTGCTGTGTTC | |
| | GAACCTAGCGAAGCTGAGATCTCTCATACACAAAAGGCTACACTGGTGTGTCTGGCTACAGGC | |
| | TTCTTTCCCGATCATGTTGAATTAAGCTGGTGGGTGAATGGAAAAGAAGTTCACTCTGGAGTT | |
| | AGCACAGATCCCCAACCCCTTAAAGAACAACCTGCTCTGAACGATAGCAGATACTGCCTTAGC | |
| | TCTAGACTTAGGGTTAGCGCCACCTTTTGGCAAAATCCCAGAAACCATTTTAGGTGCCAGGTT | |
| | CAGTTCTACGGCCTGTCTGAAAATGATGAATGGACCCAAGATAGGGCCAAACCTGTTACACAA | |
| | ATTGTTTCTGCCGAAGCTTGGGGAAGAGCTGACTGTGGATTTACATCTGTTAGCTATCAGCAA | |
| | GGCGTGCTGTCTGCCACCATCCTGTATGAAATCCTTTTAGGCAAAGCTACCCTGTATGCTGTG | |
| | TTAGTGTCTGCCTTAGTGCTTATGGCCATGGTGAAGAGAAAGGACTTC | |
| 323 | ATGGGCACAAGCCTTCTGTGTTGGATGGCTTTGTGTTTACTTGGAGCTGATCACGCTGATACA | TCR C Vβ |
| | GGCGTGTCTCAAGACCCTAGACATAAGATTACCAAGAGGGGCCAGAATGTGACATTCAGGTGT | (nt) codon |
| | GATCCTATTTCTGAGCATAACAGGCTGTATTGGTATAGGCAAACATTGGGCCAAGGCCCTGAG | optimized |
| | TTCCTTACATACTTTCAGAACGAAGCTCAACTTGAGAAAAGCAGGTTGTTATCTGATAGGTTC | |
| | AGCGCCGAAAGGCCTAAGGGATCTTTCTCTACACTTGAGATTCAGAGAACCGAACAAGGCGAT | |
| | AGCGCTATGTATCTGTGTGCCTCTACAGAACTGTCTGGAAACACCATCTACTTTGGAGAGGGC | |
| | TCTTGGCTGACAGTTGTTGAGGACTTAAACAAGGTTTTTCCTCCCGAGGTGGCTGTGTTTGAA | |
| | CCTTCTGAAGCTGAAATTTCTCACACCCAGAAGGCTACCTTAGTTTGTCTTGCCACAGGCTTT | |
| | TTTCCCGATCACGTTGAATTGAGCTGGTGGGTTAATGGCAAGGAAGTGCATTCTGGAGTTTCT | |
| | ACAGATCCCCAGCCTCTTAAGGAACAACCTGCCTTGAACGATTCTAGATACTGTCTGAGCTCT | |
| | AGGCTGAGGGTTTCTGCTACATTTTGGCAAAATCCTAGGAACCACTTCAGGTGCCAGGTGCAG | |
| | TTCTACGGATTAAGCGAAAACGATGAATGGACACAGGACAGGGCTAAACCTGTTACACAAATT | |
| | GTTTCTGCCGAAGCTTGGGGAAGAGCTGATTGTGGATTCACCTCTGTTAGCTATCAGCAAGGA | |
| | GTGCTGTCTGCCACAATCCTGTATGAGATCTTACTTGGCAAAGCCACCTTATATGCTGTTCTT | |
| | GTTAGCGCCCTTGTGTTGATGGCCATGGTGAAGAGAAAGGACTTC | |
| 324 | ATGGGCACCAGCTTACTTTGCTGGATGGCTCTGTGTTTATTAGGAGCTGATCATGCTGATACA | TCR D Vβ |
| | GGAGTGAGCCAAGATCCCAGGCATAAGATTACAAAGAGGGGCCAGAATGTGACCTTCAGGTGT | (nt) codon |
| | GACCCTATTTCTGAACACAACAGACTGTATTGGTACAGGCAGACATTAGGCCAAGGCCCTGAG | optimized |
| | TTTCTGACATATTTCCAGAACGAGGCTCAACTTGAGAAGTCTAGGCTGTTATCTGACAGGTTC | |
| | TCTGCTGAGAGGCCTAAAGGCTCTTTTAGCACATTGGAGATCCAAAGAACCGAACAGGGCGAT | |
| | TCTGCCATGTATCTTTGTGCTTCTAGCCTTGTGTCTGGCAATGAGCAATTTTTTGGACCTGGA | |
| | ACAAGGCTGACCGTGCTTGAAGACCTGAAAAACGTTTTTCCTCCTGAAGTGGCTGTGTTCGAA | |
| | CCTTCTGAAGCTGAGATTAGCCATACCCAGAAAGCCACATTAGTTTGTCTTGCCACAGGCTTT | |
| | TACCCCGATCATGTGGAACTTTCTTGGTGGGTTAATGGCAAGGAGGTTCATTCTGGAGTGTCT | |
| | ACAGACCCTCAACCTCTGAAAGAGCAACCTGCTTTGAATGACAGCAGGTATTGTTTGTCTAGC | |
| | AGGCTGAGGGTTTCTGCTACATTCTGGCAAAATCCTAGGAATCACTTCAGGTGCCAGGTGCAG | |
| | TTTTACGGACTGAGCGAAAATGATGAATGGACACAGGATAGGGCCAAACCTGTTACACAAATT | |
| | GTTTCTGCTGAGGCTTGGGGAAGAGCTGATTGTGGATTCACAAGCGAATCTTATCAGCAAGGC | |
| | GTTCTGTCTGCCACCATCCTGTATGAGATCCTTCTTGGAAAAGCTACCTTATATGCTGTGCTG | |
| | GTTTCTGCCTTGGTGCTTATGGCCATGGTTAAGAGAAAGGACAGCAGAGGC | |
| 325 | ATGGGCACCTCTCTTCTTTGCTGGATGGCTCTGTGTTTATTAGGAGCTGATCACGCCGATACA | TCR E Vβ |
| | GGAGTTTCTCAAGATCCCAGGCACAAAATTACCAAGAGGGGCCAGAACGTTACATTCAGGTGT | (nt) codon |
| | GATCCCATTTCTGAACACAATAGACTTTATTGGTACAGGCAAACCCTGGGCCAAGGCCCTGAA | optimized |
| | TTTCTGACATATTTTCAGAACGAGGCTCAACTGGAGAAGTCTAGGCTGCTGTCTGATAGATTT | |
| | TCTGCTGAGAGGCCTAAGGGCTCTTTCTCTACCCTGGAAATTCAAAGGACAGAGCAAGGAGAT | |
| | AGCGCCATGTATCTTTGTGCTAGCAGCCTTGTTTCTGGCAACGAGCAATTTTTTGGACCTGGA | |
| | ACAAGGCTGACCGTGCTTGAAGACCTGAAAAACGTTTTTCCTCCTGAGGTTGCTGTGTTTGAA | |
| | CCTTCTGAGGCTGAAATCAGCCATACCCAAAAGGCCACATTAGTTTGTCTGGCCACAGGCTTC | |
| | TATCCTGACCATGTGGAATTATCTTGGTGGGTTAACGGCAAAGAAGTGCATTCTGGAGTTTCT | |
| | ACAGATCCTCAGCCCCTTAAAGAACAGCCTGCTCTTAATGACAGCAGGTATTGCTTATCTTCT | |
| | AGGCTGAGGGTGTCTGCTACATTTTGGCAGAACCCTAGGAATCATTTTAGGTGCCAGGTGCAG | |
| | TTTTACGGACTGAGCGAGAATGATGAATGGACACAAGACAGAGCCAAACCCGTTACACAAATC | |
| | GTTTCTGCTGAAGCTTGGGGAAGAGCTGATTGTGGCTTCACATCTGAATCTTATCAGCAGGGA | |
| | GTGTTGAGCGCCACCATTCTGTACGAGATTTTGCTTGGAAAAGCCACATTGTATGCCGTGTTG | |
| | GTTTCTGCTCTGGTGTTGATGGCTATGGTGAAGAGAAAGGACAGCAGAGGC | |
| 326 | ATGGGCACAAGCTTACTGTGTTGGATGGCTCTTTGTTTGCTTGGAGCTGATCATGCTGATACA | TCR F Vβ |
| | GGAGTGTCTCAGGATCCTAGACACAAGATCACAAAGAGGGGCCAGAATGTGACATTCAGGTGT | (nt) codon |
| | GATCCCATTAGCGAACATAACAGACTTTATTGGTACAGGCAAACCCTGGGCCAAGGACCTGAG | optimized |
| | TTTCTGACATATTTTCAGAACGAGGCTCAACTGGAAAAATCTAGGCTTCTTAGCGATAGGTTC | |
| | AGCGCTGAGAGGCCTAAAGGATCTTTTAGCACCTTGGAAATCCAAAGGACCGAACAAGGCGAT | |
| | AGCGCCATGTATTTATGTGCTTCTTCTTCTAGGGCTGGAGGAGAAACACAATATTTTGGACCT | |
| | GGCACCAGGTTGTTAGTGCTGGAGGACTTGAAGAATGTTTTTCCTCCCGAGGTGGCTGTTTTC | |
| | GAACCCTCTGAAGCTGAAATTTCTCATACCCAGAAGGCCACATTAGTGTGCCTGGCTACAGGA | |
| | TTTTACCCTGATCATGTTGAGCTTTCTTGGTGGGTGAACGGAAAAGAAGTTCATTCTGGCGTG | |
| | AGCACAGATCCTCAACCTCTTAAAGAGCAACCTGCCTTGAATGACTCTAGGTATTGTTTAAGC | |
| | TCTAGGCTGAGGGTTAGCGCCACCTTTTGGCAGAATCCTAGAAATCATTTTAGGTGCCAGGTG | |
| | CAATTCTACGGCCTGTCTGAAAATGACGAATGGACACAAGACAGGGCTAAACCTGTGACACAA | |
| | ATTGTTAGCGCTGAAGCTTGGGGAAGAGCTGATTGTGGCTTTACCTCTGAAAGCTATCAGCAA | |
| | GGAGTTCTGAGCGCCACCATTCTGTATGAGATCCTGCTTGGAAAAGCTACATTGTATGCCGTT | |
| | CTTGTTTCTGCCCTGGTGTTGATGGCCATGGTTAAGAGGAAAGATAGCAGAGGC | |
| 327 | ATGGGAACCAGCCTGCTTTGTTGGATGGCTTTATGTCTTCTTGGAGCTGATCACGCTGATACA | TCR G Vβ |
| | GGAGTGTCTCAGGATCCTAGACACAAAATCACCAAGAGGGGCCAGAATGTTACATTCAGGTGT | (nt) codon |
| | GATCCCATTTCTGAGCACAATAGGCTGTACTGGTATAGGCAAACACTTGGACAAGGCCCTGAA | optimized |
| | TTCCTGACATACTTCCAGAACGAGGCTCAACTTGAGAAGTCTAGGCTGTTATCTGATAGGTTC | |
| | TCTGCTGAAAGGCCCAAAGGATCTTTTAGCACCTTGGAGATTCAGAGGACCGAACAAGGAGAC | |
| | TCTGCTATGTACCTTTGCGCTAGCTCTTTGATCAGAGGAGAAACCCAGTATTTTGGACCTGGC | |
| | ACAAGGCTTCTGGTGTTAGAGGACTTAAAAAACGTGTTCCCTCCCGAGGTTGCTGTGTTCGAA | |
| | CCTTCTGAAGCTGAAATTTCTCACACCCAGAAGGCTACATTGGTTTGTCTGGCCACAGGATTT | |
| | TATCCCGATCATGTGGAATTAAGCTGGTGGGTGAATGGCAAAGAAGTTCATTCTGGAGTTAGC | |
| | ACAGATCCCCAGCCTTTAAAGGAGCAACCTGCTCTTAATGACAGCAGATACTGCCTGTCTTCT | |
| | AGGCTTAGGGTGTCTGCTACATTTTGGCAAAACCCTAGGAACCACTTCAGGTGTCAAGTGCAG | |
| | TTTTACGGCCTTTCTGAAAATGATGAGTGGACCCAAGACAGGGCCAAACCTGTTACCCAAATT | |
| | GTTTCTGCTGAAGCTTGGGGAAGAGCTGACTGTGGCTTTACATCTGAGAGCTATCAACAAGGA | |
| | GTGCTGTCTGCCACCATCCTGTACGAAATCCTTCTTGGAAAAGCTACCTTATATGCCGTGTTA | |
| | GTGTCTGCCCTTGTGCTTATGGCCATGGTGAAGAGAAAAGATAGCAGAGGC | |
| 328 | ATGGGAACCAGCTTGTTGTGTTGGATGGCTCTTTGTCTTTTAGGAGCTGACCATGCTGATACA | TCR H Vβ |
| | GGAGTTAGCCAAGATCCCAGGCACAAAATCACCAAAAGGGGCCAGAATGTGACATTCAGGTGT | (nt) codon |
| | GACCCTATCTCTGAACACAATAGGCTTTATTGGTATAGGCAGACACTGGGCCAAGGCCCTGAA | optimized |
| | TTTCTGACATACTTTCAGAATGAGGCTCAGCTTGAGAAATCTAGGTTGCTGTCTGATAGGTTC | |
| | AGCGCTGAAAGGCCTAAAGGAAGCTTTAGCACCTTGGAGATCCAAAGGACAGAACAAGGAGAT | |
| | AGCGCCATGTATCTGTGTGCTTCTAGCACAACACTTATCACAGGATACACCTTCGGAAGCGGA | |
| | ACAAGGTTAACCGTTGTTGAGGATCTGAACAAAGTGTTTCCTCCTGAGGTTGCTGTGTTTGAG | |
| | CCCTCTGAAGCTGAGATTTCTCATACCCAGAAAGCCACATTGGTTTGCTTGGCCACAGGCTTT | |
| | TTTCCCGATCATGTGGAATTGTCTTGGTGGGTTAATGGCAAAGAAGTGCACTCTGGAGTGTCT | |
| | ACCGATCCTCAACCTCTTAAAGAACAACCTGCTCTGAACGACTCTAGGTACTGCCTTTCTTCT | |
| | AGGTTGAGGGTTTCTGCTACCTTTTGGCAGAATCCCAGGAATCATTTCAGGTGCCAGGTGCAA | |
| | TTCTATGGACTGAGCGAAAACGATGAATGGACACAGGACAGAGCTAAACCCGTTACACAAATT | |
| | GTTTCTGCTGAGGCTTGGGGAAGAGCTGATTGTGGCTTTACCTCTGTTAGCTATCAGCAAGGA | |
| | GTGCTGTCTGCCACAATCCTGTACGAAATTCTGCTTGGAAAAGCCACACTGTATGCTGTTCTG | |
| | GTGTCTGCTCTTGTGCTTATGGCTATGGTGAAGAGAAAGGACTTC | |
| 329 | ATGGGCACCAGCCTGCTTTGTTGGATGGCTTTATGTCTTTTAGGAGCTGATCACGCCGATACA | TCR I Vβ (nt) |
| | GGAGTTTCTCAGGACCCTAGACATAAGATCACAAAGAGGGGCCAGAACGTGACATTCAGGTGT | codon |
| | GATCCTATTTCTGAGCATAACAGGCTTTATTGGTACAGGCAGACCTTAGGCCAAGGACCTGAG | optimized |
| | TTTCTGACATATTTCCAGAACGAAGCCCAGCTTGAGAAATCTAGGTTGTTGTCTGATAGGTTC | |
| | TCTGCTGAGAGGCCTAAGGGATCTTTCAGCACATTGGAGATTCAGAGAACAGAGCAAGGCGAT | |
| | AGCGCCATGTATCTGTGTGCTTCTTCTTTTCTTGCTGGAGAGACACAATATTTTGGACCTGGA | |
| | ACAAGGCTTCTGGTGCTGGAGGACCTGAAAAATGTTTTTCCTCCTGAAGTGGCTGTTTTCGAG | |
| | CCTTCTGAAGCTGAAATCAGCCATACCCAAAAAGCCACACTGGTGTGTTTAGCCACAGGATTC | |
| | TATCCCGACCATGTTGAATTGTCTTGGTGGGTGAATGGCAAGGAAGTTCACTCTGGAGTTTCT | |
| | ACAGATCCCCAACCTCTTAAAGAACAGCCTGCCTTGAATGACAGCAGATATTGCCTGAGCAGC | |
| | AGGTTAAGAGTTAGCGCTACCTTTTGGCAAAATCCCAGAAACCATTTCAGGTGCCAAGTGCAG | |
| | TTCTACGGCCTTAGCGAAAATGATGAATGGACACAAGATAGGGCCAAACCCGTTACACAAATT | |
| | GTTTCTGCTGAAGCTTGGGGAAGAGCTGATTGTGGATTCACAAGCGAAAGCTATCAACAAGGC | |
| | GTGCTGTCTGCTACCATCCTGTATGAAATTCTGCTTGGCAAAGCTACATTGTATGCTGTGCTT | |
| | GTTTCTGCCCTTGTGCTGATGGCTATGGTGAAGAGAAAGGATAGCAGAGGC | |
| 330 | ATGGGAACCTCTCTGCTGTGTTGGATGGCTTTGTGTCTTTTAGGAGCTGATCACGCTGATACA | TCR J Vβ (nt) |
| | GGCGTTAGCCAAGATCCCAGACACAAAATCACAAAGAGGGGACAGAATGTTACATTCAGGTGT | codon |
| | GACCCCATCTCTGAACACAACAGGTTATATTGGTACAGGCAGACATTGGGCCAGGGACCTGAG | optimized |
| | TTTTTAACATACTTCCAGAACGAAGCTCAACTTGAGAAATCTAGGCTGCTGTCTGATAGGTTC | |
| | TCTGCCGAAAGGCCTAAAGGAAGCTTTTCTACCCTGGAGATCCAAAGAACAGAACAAGGCGAT | |
| | AGCGCTATGTATTTGTGTGCTGTTCCTGGAGGAAGCAGCTACAATGAACAATTTTTTGGACCC | |
| | GGAACCAGGCTTACCGTTTTGGAAGACCTGAAAAATGTTTTCCCTCCCGAAGTTGCTGTGTTT | |
| | GAGCCTTCTGAAGCTGAGATCTCTCATACACAAAAGGCCACCTTAGTTTGTCTGGCCACAGGA | |
| | TTTTACCCTGATCACGTTGAATTGTCTTGGTGGGTGAACGGAAAAGAAGTTCATTCTGGCGTG | |
| | TCTACAGATCCCCAACCTCTTAAGGAACAACCTGCCTTGAATGACAGCAGATACTGCCTTAGC | |
| | TCTAGACTTAGGGTGTCTGCTACATTCTGGCAGAATCCTAGGAACCACTTTAGGTGTCAGGTT | |
| | CAGTTTTACGGCCTGAGCGAAAATGATGAGTGGACACAAGATAGGGCCAAACCTGTTACACAA | |
| | ATTGTTTCTGCTGAAGCCTGGGGAAGAGCTGACTGTGGCTTTACATCTGAATCTTATCAGCAA | |
| | GGAGTGCTGAGCGCCACCATCCTGTATGAAATTTTGCTTGGCAAAGCTACATTGTATGCCGTG | |
| | CTGGTTTCTGCTCTTGTGCTTATGGCCATGGTGAAAAGAAAGGACAGCAGAGGC | |
| 331 | ATGGGCACCTCTTTACTTTGCTGGATGGCTCTGTGTTTACTTGGAGCTGATCATGCTGATACA | TCR K Vβ |
| | GGAGTGAGCCAAGATCCCAGACATAAGATCACAAAGAGGGGCCAGAATGTGACCTTCAGGTGT | (nt) codon |
| | GACCCTATTTCTGAACACAACAGACTTTATTGGTATAGGCAGACCTTGGGACAGGGCCCTGAA | optimized |
| | TTTTTAACCTACTTCCAGAACGAAGCTCAACTTGAAAAATCTAGGCTGCTGTCTGACAGGTTC | |
| | TCTGCTGAAAGGCCTAAAGGATCTTTTAGCACCCTGGAAATTCAAAGGACCGAACAAGGCGAT | |
| | AGCGCTATGTATTTATGTGCTTCTTCTCCCGGAACCGTGTACAACGAACAATTTTTTGGCCCT | |
| | GGAACCAGACTTACCGTGCTTGAGGACCTGAAAAATGTTTTTCCCCCCGAGGTTGCTGTGTTT | |
| | GAACCTTCTGAAGCTGAAATCTCTCATACCCAGAAGGCCACACTTGTTTGTCTTGCCACAGGA | |
| | TTCTATCCTGACCATGTGGAACTGTCTTGGTGGGTTAATGGCAAGGAAGTTCACTCTGGAGTG | |
| | TCTACAGATCCTCAGCCTTTAAAAGAACAGCCCGCCTTGAATGATTCTAGGTACTGTCTTAGC | |
| | TCTAGGCTTAGGGTGAGCGCTACCTTTTGGCAAAATCCTAGGAACCATTTTAGGTGCCAGGTG | |
| | CAATTCTACGGCCTTAGCGAAAATGATGAGTGGACACAAGATAGGGCCAAACCTGTTACACAA | |
| | ATTGTTTCTGCTGAAGCTTGGGGAAGAGCCGATTGTGGCTTTACAAGCGAAAGCTATCAACAA | |
| | GGCGTTCTTAGCGCCACCATCCTGTATGAGATCCTTCTTGGAAAAGCTACCTTGTATGCCGTG | |
| | TTAGTTTCTGCCCTGGTGTTGATGGCTATGGTGAAGAGAAAGGATAGCAGAGGC | |
| 332 | ATGGGCACCAGCTTACTTTGTTGGATGGCCTTATGTTTACTGGGAGCTGATCACGCTGATACA | TCR L Vβ |
| | GGAGTTAGCCAAGATCCTAGGCATAAGATCACCAAGAGGGGCCAGAATGTGACATTCAGGTGT | (nt) codon |
| | GATCCTATCTCTGAACACAACAGGCTTTATTGGTATAGGCAAACCCTGGGACAGGGACCTGAA | optimized |
| | TTTCTGACATACTTCCAGAATGAGGCTCAACTGGAGAAATCTAGACTGCTGTCTGATAGGTTC | |
| | TCTGCTGAGAGGCCTAAGGGATCTTTTAGCACATTGGAAATCCAAAGGACCGAACAAGGCGAC | |
| | AGCGCCATGTACTTATGTGCTTCTTCTTTAGGAGTGCTTGGCATTGGCTACACATTCGGATCT | |
| | GGAACCAGGTTAACAGTTGTGGAGGACCTGAATAAAGTGTTTCCTCCTGAGGTGGCTGTTTTT | |
| | GAGCCCTCTGAGGCTGAAATTTCTCATACCCAAAAAGCCACACTGGTTTGCCTGGCTACAGGA | |
| | TTTTTTCCTGATCATGTGGAGTTAAGCTGGTGGGTGAACGGAAAAGAAGTTCATTCTGGAGTT | |
| | TCTACCGATCCCCAGCCTCTGAAAGAACAACCTGCTCTGAATGACTCTAGATACTGTCTGAGC | |
| | TCTAGGCTTAGAGTGAGCGCTACCTTTTGGCAAAATCCCAGAAACCACTTTAGGTGCCAAGTG | |
| | CAGTTCTACGGCCTTTCTGAGAATGATGAGTGGACACAAGATAGGGCCAAACCTGTGACACAA | |
| | ATTGTTTCTGCTGAAGCTTGGGGAAGAGCTGACTGTGGATTTACCAGCGTTTCTTATCAACAG | |
| | GGCGTTCTTAGCGCTACCATCCTTTACGAGATTTTACTGGGCAAAGCCACACTGTATGCTGTT | |
| | TTAGTGAGCGCTCTTGTTCTGATGGCCATGGTGAAGAGAAAGGACTTC | |
| 333 | ATGGGCACCTCTTTGTTATGCTGGATGGCCCTGTGTTTATTAGGAGCTGATCATGCTGACACA | TCR M Vβ |
| | GGAGTTAGCCAAGATCCTAGACACAAAATCACCAAGAGGGGCCAGAACGTTACATTCAGGTGT | (nt) codon |
| | GATCCCATTTCTGAACATAACAGGCTGTATTGGTATAGGCAAACCCTTGGCCAAGGCCCTGAA | optimized |
| | TTTCTGACATACTTCCAGAATGAGGCTCAACTTGAGAAATCTAGACTGCTGTCTGATAGGTTC | |
| | TCTGCTGAGAGGCCTAAAGGATCTTTCAGCACCTTGGAGATCCAAAGGACAGAACAGGGAGAT | |
| | TCTGCCATGTATCTGTGTGCCAGCTCTTTAGTTGCTGGAGAAACACAATACTTCGGACCTGGA | |
| | ACAAGGCTGCTGGTGTTAGAGGACCTGAAAAATGTTTTTCCTCCCGAGGTTGCTGTTTTCGAG | |
| | CCTTCTGAAGCTGAAATTAGCCATACCCAGAAGGCCACACTTGTTTGTCTTGCCACAGGCTTT | |
| | TATCCTGATCACGTGGAGTTGTCTTGGTGGGTTAATGGCAAGGAAGTTCATAGCGGAGTTTCT | |
| | ACCGATCCTCAGCCTTTAAAAGAACAACCCGCTCTGAACGACAGCAGATATTGCTTAAGCTCT | |
| | AGGCTGAGAGTTTCTGCCACCTTTTGGCAAAATCCCAGAAACCATTTCAGGTGCCAGGTTCAG | |
| | TTCTACGGATTATCTGAGAATGACGAGTGGACACAGGACAGGGCTAAACCTGTTACACAAATT | |
| | GTTTCTGCCGAAGCTTGGGGAAGAGCTGACTGTGGATTTACATCTGAATCTTACCAACAGGGA | |
| | GTGCTGAGCGCCACCATCCTGTATGAAATCTTACTTGGAAAAGCCACCTTATATGCCGTTCTT | |
| | GTGTCTGCTTTAGTGCTGATGGCCATGGTGAAGAGGAAAGATAGCAGAGGC | |
| 334 | ATGGGCACAAGCCTGCTTTGTTGGATGGCTCTTTGTTTATTAGGCGCTGATCATGCTGATACA | TCR N Vβ |
| | GGAGTGAGCCAAGATCCTAGGCATAAGATCACAAAGAGGGGCCAGAATGTGACATTCAGGTGT | (nt) codon |
| | GATCCCATCTCTGAACATAATAGGCTGTATTGGTACAGGCAAACCCTGGGACAGGGACCTGAG | optimized |
| | TTTTTAACATACTTCCAGAACGAGGCTCAACTGGAGAAATCTAGGCTGCTTTCTGACAGGTTT | |
| | TCTGCTGAGAGGCCCAAAGGCTCTTTTTCTACCCTGGAAATTCAGAGGACAGAACAGGGAGAT | |
| | TCTGCCATGTATCTGTGTGCTAGCTCTGAGACAACCCTTTCTGAGCAATTTTTCGGACCTGGC | |
| | ACAAGACTGACCGTGTTGGAAGATCTGAAGAACGTTTTTCCTCCCGAAGTTGCCGTGTTTGAA | |
| | CCTTCTGAAGCCGAAATTTCTCACACCCAAAAGGCCACCCTTGTTTGTTTGGCTACAGGCTTT | |
| | TACCCTGATCATGTTGAACTTAGCTGGTGGGTGAACGGCAAAGAAGTGCATTCTGGAGTTTCT | |
| | ACAGATCCTCAACCCCTGAAGGAACAACCTGCCTTAAACGACTCTAGGTACTGTCTGTCTAGC | |
| | AGACTGAGAGTTTCTGCCACATTTTGGCAAAACCCTAGGAACCACTTCAGGTGCCAAGTGCAA | |
| | TTCTATGGCTTGAGCGAAAATGATGAATGGACCCAAGACAGAGCTAAGCCTGTGACCCAAATT | |
| | GTTTCTGCTGAAGCTTGGGGAAGAGCTGATTGTGGCTTTACAAGCGAATCTTACCAACAGGGC | |
| | GTTCTGTCTGCCACCATTCTGTACGAAATCTTGCTTGGAAAAGCTACCTTGTATGCTGTGTTA | |
| | GTGTCTGCCCTGGTGTTGATGGCTATGGTGAAGAGAAAAGATAGCAGAGGC | |
| 335 | ATGGGCACATCTCTGCTTTGTTGGATGGCTTTATGTCTGTTAGGCGCTGATCACGCCGATACA | TCR O Vβ |
| | GGAGTTTCTCAAGATCCTAGACACAAGATCACCAAGAGGGGCCAGAACGTTACATTCAGGTGT | (nt) codon |
| | GATCCTATTTCTGAGCATAATAGGCTTTATTGGTACAGGCAGACACTGGGACAAGGCCCTGAA | optimized |
| | TTTCTGACATACTTCCAAAACGAGGCTCAATTAGAAAAGTCTAGGCTGTTGTCTGATAGGTTC | |
| | AGCGCCGAAAGGCCTAAAGGATCTTTCTCTACCCTTGAGATCCAAAGGACAGAACAAGGAGAC | |
| | AGCGCCATGTATTTATGCGCTAGCAGCTTAACAACATTGGATACCCAGTATTTTGGCCCTGGA | |
| | ACCAGACTGACAGTGCTTGAAGATCTGAAAAACGTGTTTCCTCCTGAAGTGGCCGTGTTTGAA | |
| | CCTTCTGAAGCTGAGATTAGCCATACCCAGAAAGCTACATTAGTTTGCTTGGCCACCGGCTTT | |
| | TATCCCGATCATGTTGAATTAAGCTGGTGGGTTAACGGCAAAGAAGTGCATTCTGGAGTTTCT | |
| | ACAGATCCCCAACCTCTTAAGGAGCAACCTGCTTTGAATGACAGCAGGTATTGTCTTTCTAGC | |
| | AGGCTGAGGGTTAGCGCTACATTTTGGCAGAATCCTAGGAACCATTTCAGGTGCCAAGTGCAG | |
| | TTCTATGGCCTTAGCGAGAATGATGAATGGACACAGGACAGAGCTAAACCTGTTACACAAATT | |
| | GTGTCTGCCGAAGCTTGGGGAAGAGCTGATTGTGGCTTTACATCTGAAAGCTACCAACAAGGA | |
| | GTGCTTAGCGCCACCATCCTGTATGAGATCCTTTTAGGAAAGGCTACCTTATATGCCGTGCTT | |
| | GTGTCTGCTCTTGTTCTGATGGCTATGGTGAAGAGGAAAGACAGCAGAGGC | |
| 336 | ATGGGAACATCTCTGCTTTGTTGGATGGCTCTTTGTCTGTTAGGCGCTGATCACGCTGATACA | TCR P Vβ |
| | GGAGTTTCTCAAGATCCCAGACACAAGATCACCAAGAGGGGCCAGAATGTTACATTCAGGTGT | (nt) codon |
| | GATCCCATCTCTGAACACAATAGACTTTATTGGTACAGGCAGACCCTGGGACAAGGCCCTGAG | optimized |
| | TTTCTTACATATTTTCAGAACGAGGCTCAGCTTGAAAAATCTAGGTTGCTGTCTGATAGGTTC | |
| | AGCGCTGAGAGGCCTAAAGGATCTTTCTCTACCTTGGAGATCCAAAGAACAGAACAAGGCGAT | |
| | TCTGCTATGTACCTTTGTGCTAGCCTTACCGGCACAGTGTACAACGAGCAATTTTTTGGACCT | |
| | GGAACAAGACTGACCGTGCTGGAAGATTTGAAAAACGTTTTCCCTCCCGAGGTTGCTGTGTTT | |
| | GAGCCTTCTGAAGCTGAAATTAGCCATACCCAGAAGGCTACCCTGGTTTGTTTGGCTACAGGC | |
| | TTTTATCCTGACCATGTTGAGCTGAGCTGGTGGGTTAATGGAAAGGAGGTTCATTCTGGAGTT | |
| | TCTACAGACCCTCAGCCTCTTAAAGAGCAACCCGCTCTGAATGACTCTAGATACTGCTTGTCT | |
| | TCTAGGCTTAGGGTTTCTGCCACATTTTGGCAGAATCCTAGGAATCACTTCAGGTGCCAAGTG | |
| | CAGTTCTATGGACTGTCTGAAAATGATGAGTGGACCCAGGATAGAGCCAAACCTGTTACACAA | |
| | ATCGTTAGCGCTGAAGCTTGGGGAAGAGCTGATTGTGGCTTTACATCTGAGAGCTATCAGCAA | |
| | GGAGTGTTATCTGCCACCATCCTGTACGAGATCCTTCTTGGAAAAGCCACATTGTATGCCGTG | |
| | CTGGTTTCTGCTCTGGTTTTGATGGCTATGGTGAAGAGAAAGGACAGCAGAGGC | |
| 337 | ATGGGCACAAGCTTACTTTGTTGGATGGCTTTGTGTCTGTTGGGAGCTGATCATGCTGATACA | TCR Q Vβ |
| | GGAGTTAGCCAAGACCCTAGACACAAGATCACAAAGAGGGGCCAGAATGTGACATTCAGGTGT | (nt) codon |
| | GATCCCATTTCTGAACACAATAGGCTGTACTGGTATAGGCAAACCTTGGGACAAGGCCCTGAG | optimized |
| | TTCTTAACATACTTCCAGAACGAGGCTCAATTAGAAAAGAGCAGACTGCTGTCTGACAGGTTC | |
| | TCTGCTGAAAGGCCTAAAGGATCTTTCAGCACACTGGAGATCCAAAGAACAGAGCAGGGAGAT | |
| | TCTGCCATGTACTTATGTGCCTCTAGCTTAGTTAGGAACGAGAAACTGTTCTTTGGCTCTGGA | |
| | ACCCAGCTGTCTGTGCTTGAAGATCTGAACAAGGTTTTTCCCCCTGAGGTTGCTGTTTTTGAG | |
| | CCTTCTGAGGCTGAGATTTCTCATACCCAAAAAGCTACACTGGTGTGTTTGGCCACAGGCTTT | |
| | TTTCCTGACCACGTTGAACTTTCTTGGTGGGTGAATGGCAAGGAAGTTCACTCTGGAGTTTCT | |
| | ACAGATCCTCAGCCTCTGAAGGAACAACCTGCTCTGAACGATAGCAGATACTGTCTTAGCAGC | |
| | AGACTGAGGGTTTCTGCTACCTTTTGGCAAAATCCTAGGAACCACTTCAGATGCCAGGTGCAA | |
| | TTCTACGGCCTTTCTGAAAATGACGAATGGACCCAAGATAGGGCCAAACCTGTTACACAGATT | |
| | GTTTCTGCTGAAGCTTGGGGAAGAGCTGATTGTGGCTTCACATCTGTTAGCTATCAGCAAGGC | |
| | GTTCTTTCTGCCACCATCCTGTACGAAATCCTTCTTGGAAAAGCCACCCTGTATGCTGTTCTG | |
| | GTTTCTGCCCTTGTTTTGATGGCTATGGTGAAGAGAAAGGACTTC | |
| 338 | ATGGGCACCTCTCTTTTATGTTGGATGGCCCTTTGCTTACTTGGAGCTGATCATGCTGATACA | TCR R Vβ |
| | GGAGTGTCTCAAGATCCTAGGCACAAGATCACCAAAAGGGGCCAGAATGTTACATTCAGGTGT | (nt) codon |
| | GATCCCATCTCTGAGCATAACAGACTTTATTGGTACAGGCAGACATTAGGCCAGGGCCCTGAA | optimized |
| | TTCCTGACATACTTTCAAAACGAGGCTCAACTTGAGAAGTCTAGACTTCTGTCTGATAGGTTC | |
| | AGCGCTGAAAGGCCTAAGGGATCTTTCAGCACACTTGAGATCCAAAGAACCGAGCAAGGAGAC | |
| | TCTGCTATGTATTTATGCGCCAGCTCTCCCTTAAAGGGCAGCAATTATGGATATACCTTCGGA | |
| | TCTGGCACCAGGTTAACCGTTGTGGAGGACTTGAATAAAGTGTTCCCTCCTGAGGTGGCTGTT | |
| | TTTGAACCTAGCGAAGCTGAAATTAGCCATACCCAAAAGGCTACACTGGTGTGTCTGGCTACA | |
| | GGCTTTTTTCCTGATCATGTGGAACTTAGCTGGTGGGTGAACGGAAAAGAAGTGCATTCTGGA | |
| | GTTTCTACAGATCCCCAACCCCTTAAAGAACAGCCTGCTCTGAACGACTCTAGATACTGCTTA | |
| | AGCAGCAGATTGAGGGTTTCTGCCACCTTTTGGCAAAATCCCAGGAATCACTTCAGGTGTCAA | |
| | GTGCAGTTCTACGGCTTGAGCGAAAATGATGAGTGGACACAAGATAGGGCTAAACCTGTGACA | |
| | CAAATTGTTTCTGCCGAAGCTTGGGGAAGAGCTGATTGTGGCTTTACATCTGTGAGCTATCAG | |
| | CAAGGAGTGCTGTCTGCCACAATCCTGTACGAAATCCTTCTTGGAAAAGCCACCTTATATGCT | |
| | GTGCTGGTGTCTGCCCTTGTTTTGATGGCTATGGTGAAGAGAAAGGACTTC | |
| 339 | ATGGGCACCAGCTTACTGTGTTGGATGGCTCTTTGTTTACTTGGAGCTGATCACGCCGATACA | TCR S Vβ |
| | GGAGTTAGCCAAGATCCTAGGCATAAGATCACAAAGAGGGGCCAGAACGTTACATTCAGGTGT | (nt) codon |
| | GATCCCATCTCTGAACATAACAGGCTTTATTGGTATAGGCAGACCTTAGGACAGGGCCCTGAA | optimized |
| | TTCCTGACATACTTCCAAAACGAAGCTCAGCTTGAGAAATCTAGGCTTCTGTCTGATAGATTT | |
| | TCTGCCGAGAGGCCCAAGGGATCTTTCTCTACCTTAGAAATTCAAAGGACAGAACAAGGAGAT | |
| | AGCGCTATGTATCTGTGTGCTTCTTCTCCTGGCACCGTTTATAATGAGCAGTTTTTCGGACCT | |
| | GGAACAAGGCTGACCGTGTTGGAAGACTTGAAAAATGTTTTCCCTCCCGAAGTGGCTGTTTTT | |
| | GAGCCTTCTGAAGCTGAGATCTCTCATACCCAAAAAGCCACACTGGTTTGCTTAGCCACAGGC | |
| | TTCTACCCTGATCATGTGGAATTGTCTTGGTGGGTTAATGGCAAGGAAGTTCATAGCGGAGTT | |
| | TCTACAGATCCCCAACCCCTTAAAGAGCAACCTGCCTTGAATGATTCTAGGTACTGCTTAAGC | |
| | AGCAGACTTAGGGTGTCTGCCACATTTTGGCAGAATCCCAGAAACCATTTCAGGTGCCAAGTG | |
| | CAGTTTTATGGCCTGTCTGAAAATGATGAGTGGACACAAGATAGAGCCAAACCTGTGACACAA | |
| | ATTGTTTCTGCTGAAGCTTGGGGAAGAGCCGATTGTGGATTTACCAGCGAAAGCTATCAACAA | |
| | GGCGTTCTGTCTGCCACCATCCTGTATGAAATCTTACTTGGAAAGGCCACCTTGTATGCTGTG | |
| | TTAGTGTCTGCTCTGGTTTTGATGGCTATGGTGAAGAGAAAGGACAGCAGAGGC | |
| 340 | ATGGGAACATCTCTGTTGTGTTGGATGGCCCTGTGTTTACTTGGAGCTGATCATGCTGATACA | TCR T Vβ |
| | GGAGTTAGCCAAGACCCCAGACACAAGATTACAAAGAGGGGCCAGAATGTGACATTCAGGTGT | (nt) codon |
| | GACCCTATCTCTGAACACAATAGGCTGTATTGGTATAGGCAAACACTGGGACAGGGCCCTGAG | optimized |
| | TTTCTTACATACTTCCAGAATGAAGCCCAACTTGAGAAGTCTAGGCTGTTGTCTGACAGGTTC | |
| | TCTGCTGAAAGGCCTAAAGGATCTTTCAGCACCCTTGAGATTCAGAGGACAGAACAAGGAGAT | |
| | AGCGCCATGTATCTGTGTGCTTCTTCTCAACTTGCCGGAGTGATTGAACAATTTTTTGGACCT | |
| | GGCACAAGGCTTACCGTGCTTGAGGATTTGAAAAACGTTTTCCCTCCCGAGGTTGCTGTGTTT | |
| | GAACCTTCTGAAGCTGAGATCTCTCATACACAGAAGGCTACATTAGTGTGCCTTGCCACAGGC | |
| | TTTTATCCTGATCATGTTGAACTTAGCTGGTGGGTGAACGGCAAAGAAGTTCATTCTGGAGTT | |
| | AGCACAGATCCTCAACCTCTGAAGGAACAACCTGCTCTGAATGATTCTAGGTACTGCCTGTCT | |
| | TCTAGACTGAGGGTTTCTGCTACCTTTTGGCAGAATCCCAGAAACCACTTCAGATGCCAAGTG | |
| | CAGTTCTATGGACTGAGCGAGAACGATGAATGGACACAGGATAGAGCCAAACCTGTTACACAA | |
| | ATTGTTAGCGCTGAAGCTTGGGGAAGGGCTGATTGTGGATTTACATCTGAATCTTACCAGCAG | |
| | GGCGTTCTGAGCGCCACCATTCTGTATGAAATTTTGCTTGGAAAGGCTACCTTGTATGCCGTT | |
| | CTTGTTTCTGCTCTGGTTTTGATGGCCATGGTGAAGAGAAAGGACAGCAGAGGC | |
| 341 | MASAPISMLAMLFTLSGLR | TCR A, D, E |
| | | alpha chain |
| | | ss (aa) |
| 342 | METLLGVSLVILWLQLARVN | TCR B, G, R, |
| | | T alpha |
| | | chain ss (aa) |
| 343 | METLLGLLILWLQLQWVSS | TCR C, I, L, |
| | | M alpha |
| | | chain ss (aa) |
| 344 | MSIRAVFIFLWLQLDLVN | TCR F, H, O, |
| | | Q alpha |
| | | chain ss (aa) |
| 345 | MKSLRVLLVILWLQLSWVWSQ | TCR J alpha |
| | | chain ss (aa) |
| 346 | MKLVTSITVLLSLGIMG | TCR K alpha |
| | | chain ss (aa) |
| 347 | MSIRALFIFLWLQLDLVN | TCR N alpha |
| | | chain ss (aa) |
| 348 | MLLLLIPVLGMIFALRDAR | TCR P alpha |
| | | chain ss (aa) |
| 349 | MRLVARVTVFLTFGTII | TCR S alpha |
| | | chain ss (aa) |
| 350 | MGTSLLCWMALCLLGADHA | TCR A-T |
| | | beta chain |
| | | ss (aa) |
| 351 | GCCACGAACTTCTCTCTGTTAAAGCAAGCAGGAGACGTGGAAGAAAACCCCGGTCCC | P2A (nt) |
| 352 | ATNFSLLKQAGDVEENPGP | P2A (aa) |
| 353 | GSG | Linker (aa) |
| 354 | VLHDDLLEA | HA-1 H |
| | | variant |
| | | peptide |
| 355 | VLRDDLLEA | HA-1 R |
| | | variant |
| | | peptide |
| 356 | DKQLDADVSPKPTIFLPSIAETKLQKAGTYLCLLEKFFPDVIKIHWQEKKSNTILGSQEGNTM | TRGC1 (aa)- |
| | KTNDTYMKFSWLTVPEKSLDKEHRCIVRHENNKNGVDQEIIFPPIKTDVITMDPKDNCSKDAN | Uniprot |
| | DTLLLQLTNTSAYYMYLLLLLKSVVYFAIITCCLLRRTAFCCNGEKS | POCF51 |
| 357 | DKQLDADVSPKPTIFLPSIAETKLQKAGTYLCLLEKFFPDIIKIHWQEKKSNTILGSQEGNTM | TRGC2 (aa)- |
| | KTNDTYMKFSWLTVPEESLDKEHRCIVRHENNKNGIDQEIIFPPIKTDVTTVDPKYNYSKDAN | Uniprot |
| | DVITMDPKDNWSKDANDTLLLQLTNTSAYYTYLLLLLKSVVYFAIITCCLLRRTAFCCNGEKS | P03986 |
| 358 | SQPHTKPSVFVMKNGTNVACLVKEFYPKDIRINLVSSKKITEFDPAIVISPSGKYNAVKLGKY | TRDC (aa)- |
| | EDSNSVTCSVQHDNKTVHSTDFEVKTDSTDHVKPKETENTKQPSKSCHKPKAIVHTEKVNMMS | Uniprot |
| | LTVLGLRMLFAKTVAVNFLLTAKLFFL | B7Z8K6 |
| 359 | AGHSSGTYKYI | TCR U Vα |
| | | CDR-3 |
| 360 | QQVMQIPQYQHVQEGEDFTTYCNSSTTLSNIQWYKQRPGGHPVFLIQLVKSGEVKKQKRLTFQ | TCR U Vα |
| | FGEAKKNSSLHITATQTTDVGTYFCAGHSSGTYKYIFGTGTRLKVLAN | (aa) |
| 361 | MLLITSMLVLWMQLSQVNGQQVMQIPQYQHVQEGEDFTTYCNSSTTLSNIQWYKQRPGGHPVF | TCR U Vα + |
| | LIQLVKSGEVKKQKRLTFQFGEAKKNSSLHITATQTTDVGTYFCAGHSSGTYKYIFGTGTRLK | ss (aa) |
| | VLAN | |
| 362 | MLLITSMLVLWMQLSQVNGQQVMQIPQYQHVQEGEDFTTYCNSSTTLSNIQWYKQRPGGHPVF | TCR U Vα + |
| | LIQLVKSGEVKKQKRLTFQFGEAKKNSSLHITATQTTDVGTYFCAGHSSGTYKYIFGTGTRLK | Cα (aa) |
| | VLANIQNPDPAVYQLRDSKSSDKSVCLFTDFDSQTNVSQSKDSDVYITDKTVLDMRSMDFKSN | |
| | SAVAWSNKSDFACANAFNNSIIPEDTFFPSPESSCDVKLVEKSFETDTNLNFQNLSVIGFRIL | |
| | LLKVAGFNLLMTLRLWSS | |
| 363 | ASSPGTVLNEQF | TCR U Vβ |
| | | CDR-3 |
| 364 | DTGVSQDPRHKITKRGQNVTFRCDPISEHNRLYWYRQTLGQGPEFLTYFQNEAQLEKSRLLSD | TCR U Vβ |
| | RFSAERPKGSFSTLEIQRTEQGDSAMYLCASSPGTVLNEQFFGPGTRLTVLE | (aa) |
| 365 | MGTSLLCWMALCLLGADHADTGVSQDPRHKITKRGQNVTFRCDPISEHNRLYWYRQTLGQGPE | TCR U Vβ + |
| | FLTYFQNEAQLEKSRLLSDRFSAERPKGSFSTLEIQRTEQGDSAMYLCASSPGTVLNEQFFGP | ss (aa) |
| | GTRLTVLE | |
| 366 | MGTSLLCWMALCLLGADHADTGVSQDPRHKITKRGQNVTFRCDPISEHNRLYWYRQTLGQGPE | TCR U Vβ + |
| | FLTYFQNEAQLEKSRLLSDRESAERPKGSFSTLEIQRTEQGDSAMYLCASSPGTVLNEQFFGP | Cß (aa) |
| | GTRLTVLEDLKNVFPPEVAVFEPSEAEISHTQKATLVCLATGFYPDHVELSWWVNGKEVHSGV | |
| | STDPQPLKEQPALNDSRYCLSSRLRVSATFWQNPRNHFRCQVQFYGLSENDEWTQDRAKPVTQ | |
| | IVSAEAWGRADCGFTSESYQQGVLSATILYEILLGKATLYAVLVSALVLMAMVKRKDSRG | |
| 367 | MLLITSMLVLWMQLSQVNGQQVMQIPQYQHVQEGEDFTTYCNSSTTLSNIQWYKQRPGGHPVF | TCR U |
| | LIQLVKSGEVKKQKRLTFQFGEAKKNSSLHITATQTTDVGTYFCAGHSSGTYKYIFGTGTRLK | protein |
| | VLANIQNPDPAVYQLRDSKSSDKSVCLFTDFDSQTNVSQSKDSDVYITDKTVLDMRSMDFKSN | |
| | SAVAWSNKSDFACANAFNNSIIPEDTFFPSPESSCDVKLVEKSFETDTNLNFQNLSVIGFRIL | |
| | LLKVAGFNLLMTLRLWSSGSGATNFSLLKQAGDVEENPGPMGTSLLCWMALCLLGADHADTGV | |
| | SQDPRHKITKRGQNVTFRCDPISEHNRLYWYRQTLGQGPEFLTYFQNEAQLEKSRLLSDRESA | |
| | ERPKGSFSTLEIQRTEQGDSAMYLCASSPGTVLNEQFFGPGTRLTVLEDLKNVFPPEVAVFEP | |
| | SEAEISHTQKATLVCLATGFYPDHVELSWWVNGKEVHSGVSTDPQPLKEQPALNDSRYCLSSR | |
| | LRVSATFWQNPRNHERCQVQFYGLSENDEWTQDRAKPVTQIVSAEAWGRADCGFTSESYQQGV | |
| | LSATILYEILLGKATLYAVLVSALVLMAMVKRKDSRG | |
| 368 | ATGCTACTCATCACATCAATGTTGGTCTTATGGATGCAATTGTCACAGGTGAATGGACAACAG | TCR U (nt) |
| | GTAATGCAAATTCCTCAGTACCAGCATGTACAAGAAGGAGAGGACTTCACCACGTACTGCAAT | |
| | TCCTCAACTACTTTAAGCAATATACAGTGGTATAAGCAAAGGCCTGGTGGACATCCCGTTTTT | |
| | TTGATACAGTTAGTGAAGAGTGGAGAAGTGAAGAAGCAGAAAAGACTGACATTTCAGTTTGGA | |
| | GAAGCAAAAAAGAACAGCTCCCTGCACATCACAGCCACCCAGACTACAGATGTAGGAACCTAC | |
| | TTCTGTGCAGGGCACTCCTCAGGAACCTACAAATACATCTTTGGAACAGGCACCAGGCTGAAG | |
| | GTTTTAGCAAATATCCAGAACCCTGACCCTGCCGTGTACCAGCTGAGAGACTCTAAATCCAGT | |
| | GACAAGTCTGTCTGCCTATTCACCGATTTTGATTCTCAAACAAATGTGTCACAAAGTAAGGAT | |
| | TCTGATGTGTATATCACAGACAAAACTGTGCTAGACATGAGGTCTATGGACTTCAAGAGCAAC | |
| | AGTGCTGTGGCCTGGAGCAACAAATCTGACTTTGCATGTGCAAACGCCTTCAACAACAGCATT | |
| | ATTCCAGAAGACACCTTCTTCCCCAGCCCAGAAAGTTCCTGTGATGTCAAGCTGGTCGAGAAA | |
| | AGCTTTGAAACAGATACGAACCTAAACTTTCAAAACCTGTCAGTGATTGGGTTCCGAATCCTC | |
| | CTCCTGAAAGTGGCCGGGTTTAATCTGCTCATGACGCTGCGGCTGTGGTCCAGCGGCTCCGGA | |
| | GCCACGAACTTCTCTCTGTTAAAGCAAGCAGGAGACGTGGAAGAAAACCCCGGTCCCATGGGC | |
| | ACCAGCCTCCTCTGCTGGATGGCCCTGTGTCTCCTGGGGGCAGATCACGCAGATACTGGAGTC | |
| | TCCCAGGACCCCAGACACAAGATCACAAAGAGGGGACAGAATGTAACTTTCAGGTGTGATCCA | |
| | ATTTCTGAACACAACCGCCTTTATTGGTACCGACAGACCCTGGGGCAGGGCCCAGAGTTTCTG | |
| | ACTTACTTCCAGAATGAAGCTCAACTAGAAAAATCAAGGCTGCTCAGTGATCGGTTCTCTGCA | |
| | GAGAGGCCTAAGGGATCTTTCTCCACCTTGGAGATCCAGCGCACAGAGCAGGGGGACTCGGCC | |
| | ATGTATCTCTGTGCCAGCAGCCCCGGGACAGTTCTCAATGAGCAGTTCTTCGGGCCAGGGACA | |
| | CGGCTCACCGTGCTAGAGGACCTGAAAAACGTGTTCCCACCCGAGGTCGCTGTGTTTGAGCCA | |
| | TCAGAAGCAGAGATCTCCCACACCCAAAAGGCCACACTGGTGTGCCTGGCCACAGGCTTCTAC | |
| | CCCGACCACGTGGAGCTGAGCTGGTGGGTGAATGGGAAGGAGGTGCACAGTGGGGTCAGCACA | |
| | GACCCGCAGCCCCTCAAGGAGCAGCCCGCCCTCAATGACTCCAGATACTGCCTGAGCAGCCGC | |
| | CTGAGGGTCTCGGCCACCTTCTGGCAGAACCCCCGCAACCACTTCCGCTGTCAAGTCCAGTTC | |
| | TACGGGCTCTCGGAGAATGACGAGTGGACCCAGGATAGGGCCAAACCTGTCACCCAGATCGTC | |
| | AGCGCCGAGGCCTGGGGTAGAGCAGACTGTGGCTTCACCTCCGAGTCTTACCAGCAAGGGGTC | |
| | CTGTCTGCCACCATCCTCTATGAGATCTTGCTAGGGAAGGCCACCTTGTATGCCGTGCTGGTC | |
| | AGTGCCCTCGTGCTGATGGCCATGGTCAAGAGAAAGGATTCCAGAGGCTGA | |
| | α chain nucleotides: 1-819 | |
| | P2A nucleotides: 820-876 | |
| | ß chain nucleotides: 877-1815 | |
| 369 | ALYSGAGSYQLT | TCR V Vα |
| | | CDR-3 |
| 370 | MAQTVTQSQPEMSVQEAETVTLSCTYDTSENNYYLFWYKQPPSRQMILVIRQEAYKQQNATEN | TCR V Vα |
| | RFSVNFQKAAKSFSLKISDSQLGDTAMYFCALYSGAGSYQLTFGKGTKLSVIPN | (aa) |
| 371 | MTRVSLLWAVVVSTCLESGMAQTVTQSQPEMSVQEAETVTLSCTYDTSENNYYLFWYKQPPSR | TCR V Vα + |
| | QMILVIRQEAYKQQNATENRFSVNFQKAAKSFSLKISDSQLGDTAMYFCALYSGAGSYQLTFG | ss (aa) |
| | KGTKLSVIPN | |
| 372 | MTRVSLLWAVVVSTCLESGMAQTVTQSQPEMSVQEAETVTLSCTYDTSENNYYLFWYKOPPSR | TCR V Vα + |
| | QMILVIRQEAYKQQNATENRFSVNFQKAAKSFSLKISDSQLGDTAMYFCALYSGAGSYQLTFG | Cα (aa) |
| | KGTKLSVIPNIQNPDPAVYQLRDSKSSDKSVCLFTDFDSQTNVSQSKDSDVYITDKTVLDMRS | |
| | MDFKSNSAVAWSNKSDFACANAENNSIIPEDTFFPSPESSCDVKLVEKSFETDTNLNFQNLSV | |
| | IGFRILLLKVAGFNLLMTLRLWSS | |
| 373 | ASSLAAGMNTEAF | TCR V Vβ |
| | | CDR-3 |
| 374 | DTGVSQDPRHKITKRGQNVTFRCDPISEHNRLYWYRQTLGQGPEFLTYFQNEAQLEKSRLLSD | TCR V Vβ |
| | RFSAERPKGSFSTLEIQRTEQGDSAMYLCASSLAAGMNTEAFFGQGTRLTVVE | (aa) |
| 375 | MGTSLLCWMALCLLGADHADTGVSQDPRHKITKRGONVTFRCDPISEHNRLYWYRQTLGQGPE | TCR V Vβ + |
| | FLTYFQNEAQLEKSRLLSDRFSAERPKGSFSTLEIQRTEQGDSAMYLCASSLAAGMNTEAFFG | ss (aa) |
| | QGTRLTVVE | |
| 376 | MGTSLLCWMALCLLGADHADTGVSQDPRHKITKRGQNVTFRCDPISEHNRLYWYROTLGQGPE | TCR V Vβ + |
| | FLTYFQNEAQLEKSRLLSDRFSAERPKGSFSTLEIQRTEQGDSAMYLCASSLAAGMNTEAFFG | Cß (aa) |
| | QGTRLTVVEDLNKVFPPEVAVFEPSEAEISHTQKATLVCLATGFFPDHVELSWWVNGKEVHSG | |
| | VSTDPQPLKEQPALNDSRYCLSSRLRVSATFWQNPRNHFRCQVQFYGLSENDEWTQDRAKPVT | |
| | QIVSAEAWGRADCGFTSVSYQQGVLSATILYEILLGKATLYAVLVSALVLMAMVKRKDF | |
| 377 | MTRVSLLWAVVVSTCLESGMAQTVTQSQPEMSVQEAETVTLSCTYDTSENNYYLFWYKQPPSR | TCR V |
| | QMILVIRQEAYKQQNATENRFSVNFQKAAKSFSLKISDSQLGDTAMYFCALYSGAGSYQLTFG | protein |
| | KGTKLSVIPNIQNPDPAVYQLRDSKSSDKSVCLFTDFDSQTNVSQSKDSDVYITDKTVLDMRS | |
| | MDFKSNSAVAWSNKSDFACANAFNNSIIPEDTFFPSPESSCDVKLVEKSFETDTNLNFQNLSV | |
| | IGFRILLLKVAGFNLLMTLRLWSSGSGATNFSLLKQAGDVEENPGPMGTSLLCWMALCLLGAD | |
| | HADTGVSQDPRHKITKRGQNVTFRCDPISEHNRLYWYRQTLGQGPEFLTYFQNEAQLEKSRLL | |
| | SDRFSAERPKGSFSTLEIQRTEQGDSAMYLCASSLAAGMNTEAFFGQGTRLTVVEDLNKVFPP | |
| | EVAVFEPSEAEISHTQKATLVCLATGFFPDHVELSWWVNGKEVHSGVSTDPQPLKEQPALNDS | |
| | RYCLSSRLRVSATFWQNPRNHFRCQVQFYGLSENDEWTQDRAKPVTQIVSAEAWGRADCGFTS | |
| | VSYQQGVLSATILYEILLGKATLYAVLVSALVLMAMVKRKDF | |
| 378 | ATGACACGAGTTAGCTTGCTGTGGGCAGTCGTGGTCTCCACCTGTCTTGAATCCGGCATGGCC | TCR V (nt) |
| | CAGACAGTCACTCAGTCTCAACCAGAGATGTCTGTGCAGGAGGCAGAGACTGTGACCCTGAGT | |
| | TGCACATATGACACCAGTGAGAATAATTATTATTTGTTCTGGTACAAGCAGCCTCCCAGCAGG | |
| | CAGATGATTCTCGTTATTCGCCAAGAAGCTTATAAGCAACAGAATGCAACGGAGAATCGTTTC | |
| | TCTGTGAACTTCCAGAAAGCAGCCAAATCCTTCAGTCTCAAGATCTCAGACTCACAGCTGGGG | |
| | GACACTGCGATGTATTTCTGTGCTTTATACTCTGGGGCTGGGAGTTACCAACTCACTTTCGGG | |
| | AAGGGGACCAAACTCTCGGTCATACCAAATATCCAGAACCCTGACCCTGCCGTGTACCAGCTG | |
| | AGAGACTCTAAATCCAGTGACAAGTCTGTCTGCCTATTCACCGATTTTGATTCTCAAACAAAT | |
| | GTGTCACAAAGTAAGGATTCTGATGTGTATATCACAGACAAAACTGTGCTAGACATGAGGTCT | |
| | ATGGACTTCAAGAGCAACAGTGCTGTGGCCTGGAGCAACAAATCTGACTTTGCATGTGCAAAC | |
| | GCCTTCAACAACAGCATTATTCCAGAAGACACCTTCTTCCCCAGCCCAGAAAGTTCCTGTGAT | |
| | GTCAAGCTGGTCGAGAAAAGCTTTGAAACAGATACGAACCTAAACTTTCAAAACCTGTCAGTG | |
| | ATTGGGTTCCGAATCCTCCTCCTGAAAGTGGCCGGGTTTAATCTGCTCATGACGCTGCGGCTG | |
| | TGGTCCAGCGGCTCCGGAGCCACGAACTTCTCTCTGTTAAAGCAAGCAGGAGACGTGGAAGAA | |
| | AACCCCGGTCCCATGGGCACCAGCCTCCTCTGCTGGATGGCCCTGTGTCTCCTGGGGGCAGAT | |
| | CACGCAGATACTGGAGTCTCCCAGGACCCCAGACACAAGATCACAAAGAGGGGACAGAATGTA | |
| | ACTTTCAGGTGTGATCCAATTTCTGAACACAACCGCCTTTATTGGTACCGACAGACCCTGGGG | |
| | CAGGGCCCAGAGTTTCTGACTTACTTCCAGAATGAAGCTCAACTAGAAAAATCAAGGCTGCTC | |
| | AGTGATCGGTTCTCTGCAGAGAGGCCTAAGGGATCTTTCTCCACCTTGGAGATCCAGCGCACA | |
| | GAGCAGGGGGACTCGGCCATGTATCTCTGTGCCAGCAGCTTAGCGGCAGGGATGAACACTGAA | |
| | GCTTTCTTTGGACAAGGCACCAGACTCACAGTTGTAGAGGACCTGAACAAGGTGTTCCCACCC | |
| | GAGGTCGCTGTGTTTGAGCCATCAGAAGCAGAGATCTCCCACACCCAAAAGGCCACACTGGTG | |
| | TGCCTGGCCACAGGCTTCTTCCCCGACCACGTGGAGCTGAGCTGGTGGGTGAATGGGAAGGAG | |
| | GTGCACAGTGGGGTCAGCACGGACCCGCAGCCCCTCAAGGAGCAGCCCGCCCTCAATGACTCC | |
| | AGATACTGCCTGAGCAGCCGCCTGAGGGTCTCGGCCACCTTCTGGCAGAACCCCCGCAACCAC | |
| | TTCCGCTGTCAAGTCCAGTTCTACGGGCTCTCGGAGAATGACGAGTGGACCCAGGATAGGGCC | |
| | AAACCCGTCACCCAGATCGTCAGCGCCGAGGCCTGGGGTAGAGCAGACTGTGGCTTTACCTCG | |
| | GTGTCCTACCAGCAAGGGGTCCTGTCTGCCACCATCCTCTATGAGATCCTGCTAGGGAAGGCC | |
| | ACCCTGTATGCTGTGCTGGTCAGCGCCCTTGTGTTGATGGCCATGGTCAAGAGAAAGGATTTC | |
| | TGA | |
| | α chain nucleotides: 1-837 | |
| | P2A nucleotides: 838-894 | |
| | ß chain nucleotides: 895-1830 | |
| 379 | CAVRDPTSGTYKYIF | TCR W Vα |
| | | CDR-3 |
| 380 | AQSVAQPEDQVNVAEGNPLTVKCTYSVSGNPYLFWYVQYPNRGLQFLLKYITGDNLVKGSYGF | TCR Vα (aa) |
| | EAEFNKSQTSFHLKKPSALVSDSALYFCAVRDPTSGTYKYIFGTGTRLKVLAN | |
| 381 | MASAPISMLAMLFTLSGLRAQSVAQPEDQVNVAEGNPLTVKCTYSVSGNPYLFWYVQYPNRGL | TCR W Vα + |
| | QFLLKYITGDNLVKGSYGFEAEFNKSQTSFHLKKPSALVSDSALYFCAVRDPTSGTYKYIFGT | ss (aa) |
| | GTRLKVLAN | |
| 382 | MASAPISMLAMLFTLSGLRAQSVAQPEDQVNVAEGNPLTVKCTYSVSGNPYLFWYVQYPNRGL | TCR W Vα + |
| | QFLLKYITGDNLVKGSYGFEAEFNKSQTSFHLKKPSALVSDSALYFCAVRDPTSGTYKYIFGT | Cα (aa) |
| | GTRLKVLANIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGFNLLMTLRLWSS | |
| 383 | CASSLVQGNEQFF | TCR W Vβ |
| | | CDR-3 |
| 384 | DTGVSQDPRHKITKRGQNVTFRCDPISEHNRLYWYRQTLGQGPEFLTYFQNEAQLEKSRLLSD | TCR W Vβ |
| | RFSAERPKGSFSTLEIQRTEQGDSAMYLCASSLVQGNEQFFGPGTRLTVLE | (aa) |
| 385 | MGTSLLCWMALCLLGADHADTGVSQDPRHKITKRGQNVTFRCDPISEHNRLYWYRQTLGQGPE | TCR W Vβ + |
| | FLTYFQNEAQLEKSRLLSDRFSAERPKGSFSTLEIQRTEQGDSAMYLCASSLVQGNEQFFGPG | ss (aa) |
| | TRLTVLE | |
| 386 | MGTSLLCWMALCLLGADHADTGVSQDPRHKITKRGQNVTFRCDPISEHNRLYWYRQTLGQGPE | TCR W Vβ + |
| | FLTYFQNEAQLEKSRLLSDRFSAERPKGSFSTLEIQRTEQGDSAMYLCASSLVQGNEQFFGPG | Cß (aa) |
| | TRLTVLEDLKNVEPPEVAVFEPSEAEISHTQKATLVCLATGFYPDHVELSWWVNGKEVHSGVS | |
| | TDPQPLKEQPALNDSRYCLSSRLRVSATFWQNPRNHFRCQVQFYGLSENDEWTQDRAKPVTQI | |
| | VSAEAWGRADCGFTSESYQQGVLSATILYEILLGKATLYAVLVSALVLMAMVKRKDSRG | |
| 387 | MASAPISMLAMLFTLSGLRAQSVAQPEDQVNVAEGNPLTVKCTYSVSGNPYLFWYVQYPNRGL | TCR W |
| | QFLLKYITGDNLVKGSYGFEAEFNKSQTSFHLKKPSALVSDSALYFCAVRDPTSGTYKYIFGT | protein |
| | GTRLKVLANIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGFNLLMTLRLWSSGSGATNFSLLKQAGDVEENPGPMGTSLLCWMALCLLGADH | |
| | ADTGVSQDPRHKITKRGQNVTFRCDPISEHNRLYWYRQTLGQGPEFLTYFQNEAQLEKSRLLS | |
| | DRFSAERPKGSFSTLEIQRTEQGDSAMYLCASSLVQGNEQFFGPGTRLTVLEDLKNVFPPEVA | |
| | VFEPSEAEISHTQKATLVCLATGFYPDHVELSWWVNGKEVHSGVSTDPQPLKEQPALNDSRYC | |
| | LSSRLRVSATFWQNPRNHFRCQVQFYGLSENDEWTQDRAKPVTQIVSAEAWGRADCGFTSESY | |
| | QQGVLSATILYEILLGKATLYAVLVSALVLMAMVKRKDSRG | |
| 388 | ATGGCCTCTGCACCCATCTCGATGCTTGCGATGCTCTTCACATTGAGTGGGCTGAGAGCTCAG | TCR W (nt) |
| | TCAGTGGCTCAGCCGGAAGATCAGGTCAACGTTGCTGAAGGGAATCCTCTGACTGTGAAATGC | |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGACCCTACCTCAGGAACCTACAAATACATCTTTGGAACA | |
| | GGCACCAGGCTGAAGGTTTTAGCAAATATCCAGAACCCTGACCCTGCCGTGTACCAGCTGAGA | |
| | GACTCTAAATCCAGTGACAAGTCTGTCTGCCTATTCACCGATTTTGATTCTCAAACAAATGTG | |
| | TCACAAAGTAAGGATTCTGATGTGTATATCACAGACAAAACTGTGCTAGACATGAGGTCTATG | |
| | GACTTCAAGAGCAACAGTGCTGTGGCCTGGAGCAACAAATCTGACTTTGCATGTGCAAACGCC | |
| | TTCAACAACAGCATTATTCCAGAAGACACCTTCTTCCCCAGCCCAGAAAGTTCCTGTGATGTC | |
| | AAGCTGGTCGAGAAAAGCTTTGAAACAGATACGAACCTAAACTTTCAAAACCTGTCAGTGATT | |
| | GGGTTCCGAATCCTCCTCCTGAAAGTGGCCGGGTTTAATCTGCTCATGACGCTGCGGCTGTGG | |
| | TCCAGCGGCTCCGGAGCCACGAACTTCTCTCTGTTAAAGCAAGCAGGAGACGTGGAAGAAAAC | |
| | CCCGGTCCCATGGGCACCAGCCTCCTCTGCTGGATGGCCCTGTGTCTCCTGGGGGCAGATCAC | |
| | GCAGATACTGGAGTCTCCCAGGACCCCAGACACAAGATCACAAAGAGGGGACAGAATGTAACT | |
| | TTCAGGTGTGATCCAATTTCTGAACACAACCGCCTTTATTGGTACCGACAGACCCTGGGGCAG | |
| | GGCCCAGAGTTTCTGACTTACTTCCAGAATGAAGCTCAACTAGAAAAATCAAGGCTGCTCAGT | |
| | GATCGGTTCTCTGCAGAGAGGCCTAAGGGATCTTTCTCCACCTTGGAGATCCAGCGCACAGAG | |
| | CAGGGGGACTCGGCCATGTATCTCTGTGCCAGCAGCTTAGTGCAGGGCAATGAGCAGTTCTTC | |
| | GGGCCAGGGACACGGCTCACCGTGCTAGAGGACCTGAAAAACGTGTTCCCACCCGAGGTCGCT | |
| | GTGTTTGAGCCATCAGAAGCAGAGATCTCCCACACCCAAAAGGCCACACTGGTGTGCCTGGCC | |
| | ACAGGCTTCTACCCCGACCACGTGGAGCTGAGCTGGTGGGTGAATGGGAAGGAGGTGCACAGT | |
| | GGGGTCAGCACAGACCCGCAGCCCCTCAAGGAGCAGCCCGCCCTCAATGACTCCAGATACTGC | |
| | CTGAGCAGCCGCCTGAGGGTCTCGGCCACCTTCTGGCAGAACCCCCGCAACCACTTCCGCTGT | |
| | CAAGTCCAGTTCTACGGGCTCTCGGAGAATGACGAGTGGACCCAGGATAGGGCCAAACCTGTC | |
| | ACCCAGATCGTCAGCGCCGAGGCCTGGGGTAGAGCAGACTGTGGCTTCACCTCCGAGTCTTAC | |
| | CAGCAAGGGGTCCTGTCTGCCACCATCCTCTATGAGATCTTGCTAGGGAAGGCCACCTTGTAT | |
| | GCCGTGCTGGTCAGTGCCCTCGTGCTGATGGCCATGGTCAAGAGAAAGGATTCCAGAGGCTGA | |
| | α chain nucleotides: 1-834 | |
| | P2A nucleotides: 835-891 | |
| | ß chain nucleotides: 892-1827 | |
| 389 | VVLGGAGNMLT | TCR X Vα |
| | | CDR-3 |
| 390 | GENVEQHPSTLSVQEGDSAVIKCTYSDSASNYFPWYKQELGKRPQLIIDIRSNVGEKKDQRIA | TCR X Vα |
| | VTLNKTAKHFSLHITETQPEDSAVYFCVVLGGAGNMLTFGGGTRLMVKPH | (aa) |
| 391 | MTSIRAVFIFLWLQLDLVNGENVEQHPSTLSVQEGDSAVIKCTYSDSASNYFPWYKQELGKRP | TCR X Vα + |
| | QLIIDIRSNVGEKKDQRIAVTLNKTAKHFSLHITETQPEDSAVYFCVVLGGAGNMLTFGGGTR | ss (aa) |
| | LMVKPH | |
| 392 | MTSIRAVFIFLWLQLDLVNGENVEQHPSTLSVQEGDSAVIKCTYSDSASNYFPWYKQELGKRP | TCR X Vα + |
| | QLIIDIRSNVGEKKDQRIAVTLNKTAKHFSLHITETQPEDSAVYFCVVLGGAGNMLTFGGGTR | Cα (aa) |
| | LMVKPHIQNPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGFR | |
| | ILLLKVAGFNLLMTLRLWSS | |
| 393 | ASSLTVSDGYT | TCR X Vβ |
| | | CDR-3 |
| 394 | DTGVSQDPRHKITKRGQNVTFRCDPISEHNRLYWYRQTLGQGPEFLTYFQNEAQLEKSRLLSD | TCR X Vβ |
| | RFSAERPKGSFSTLEIQRTEQGDSAMYLCASSLTVSDGYTFGSGTRLTVVE | (aa) |
| 395 | MGTSLLCWMALCLLGADHADTGVSQDPRHKITKRGQNVTFRCDPISEHNRLYWYRQTLGQGPE | TCR X Vβ + |
| | FLTYFQNEAQLEKSRLLSDRFSAERPKGSFSTLEIQRTEQGDSAMYLCASSLTVSDGYTFGSG | ss (aa) |
| | TRLTVVE | |
| 396 | MGTSLLCWMALCLLGADHADTGVSQDPRHKITKRGQNVTFRCDPISEHNRLYWYRQTLGQGPE | TCR X Vβ + |
| | FLTYFQNEAQLEKSRLLSDRFSAERPKGSFSTLEIQRTEQGDSAMYLCASSLTVSDGYTFGSG | Cß (aa) |
| | TRLTVVEDLNKVFPPEVAVFEPSEAEISHTQKATLVCLATGFFPDHVELSWWVNGKEVHSGVS | |
| | TDPQPLKEQPALNDSRYCLSSRLRVSATFWQNPRNHFRCQVQFYGLSENDEWTQDRAKPVTQI | |
| | VSAEAWGRADCGFTSVSYQQGVLSATILYEILLGKATLYAVLVSALVLMAMVKRKDF | |
| 397 | MTSIRAVFIFLWLQLDLVNGENVEQHPSTLSVQEGDSAVIKCTYSDSASNYFPWYKQELGKRP | TCR X |
| | QLIIDIRSNVGEKKDQRIAVTLNKTAKHFSLHITETQPEDSAVYFCVVLGGAGNMLTFGGGTR | protein |
| | LMVKPHIQNPDPAVYQLRDSKSSDKSVCLFTDFDSQTNVSQSKDSDVYITDKTVLDMRSMDFK | |
| | SNSAVAWSNKSDFACANAFNNSIIPEDTFFPSPESSCDVKLVEKSFETDTNLNFQNLSVIGER | |
| | ILLLKVAGFNLLMTLRLWSSGSGATNFSLLKQAGDVEENPGPMGTSLLCWMALCLLGADHADT | |
| | GVSQDPRHKITKRGQNVTFRCDPISEHNRLYWYRQTLGQGPEFLTYFQNEAQLEKSRLLSDRF | |
| | SAERPKGSFSTLEIQRTEQGDSAMYLCASSLTVSDGYTFGSGTRLTVVEDLNKVEPPEVAVFE | |
| | PSEAEISHTQKATLVCLATGFFPDHVELSWWVNGKEVHSGVSTDPQPLKEQPALNDSRYCLSS | |
| | RLRVSATFWQNPRNHFRCQVQFYGLSENDEWTQDRAKPVTQIVSAEAWGRADCGFTSVSYQQG | |
| | VLSATILYEILLGKATLYAVLVSALVLMAMVKRKDF | |
| 398 | ATGACATCCATTCGAGCTGTATTTATATTCCTGTGGCTGCAGCTGGACTTGGTGAATGGAGAG | TCR X (nt) |
| | AATGTGGAGCAGCATCCTTCAACCCTGAGTGTCCAGGAGGGAGACAGCGCTGTTATCAAGTGT | |
| | ACTTATTCAGACAGTGCCTCAAACTACTTCCCTTGGTATAAGCAAGAACTTGGAAAAAGACCT | |
| | CAGCTTATTATAGACATTCGTTCAAATGTGGGCGAAAAGAAAGACCAACGAATTGCTGTTACA | |
| | TTGAACAAGACAGCCAAACATTTCTCCCTGCACATCACAGAGACCCAACCTGAAGACTCGGCT | |
| | GTCTACTTCTGTGTCGTGCTAGGGGGGGCAGGCAACATGCTCACCTTTGGAGGGGGAACAAGG | |
| | TTAATGGTCAAACCCCATATCCAGAACCCTGACCCTGCCGTGTACCAGCTGAGAGACTCTAAA | |
| | TCCAGTGACAAGTCTGTCTGCCTATTCACCGATTTTGATTCTCAAACAAATGTGTCACAAAGT | |
| | AAGGATTCTGATGTGTATATCACAGACAAAACTGTGCTAGACATGAGGTCTATGGACTTCAAG | |
| | AGCAACAGTGCTGTGGCCTGGAGCAACAAATCTGACTTTGCATGTGCAAACGCCTTCAACAAC | |
| | AGCATTATTCCAGAAGACACCTTCTTCCCCAGCCCAGAAAGTTCCTGTGATGTCAAGCTGGTC | |
| | GAGAAAAGCTTTGAAACAGATACGAACCTAAACTTTCAAAACCTGTCAGTGATTGGGTTCCGA | |
| | ATCCTCCTCCTGAAAGTGGCCGGGTTTAATCTGCTCATGACGCTGCGGCTGTGGTCCAGCGGC | |
| | TCCGGAGCCACGAACTTCTCTCTGTTAAAGCAAGCAGGAGACGTGGAAGAAAACCCCGGTCCC | |
| | ATGGGCACCAGCCTCCTCTGCTGGATGGCCCTGTGTCTCCTGGGGGCAGATCACGCAGATACT | |
| | GGAGTCTCCCAGGACCCCAGACACAAGATCACAAAGAGGGGACAGAATGTAACTTTCAGGTGT | |
| | GATCCAATTTCTGAACACAACCGCCTTTATTGGTACCGACAGACCCTGGGGCAGGGCCCAGAG | |
| | TTTCTGACTTACTTCCAGAATGAAGCTCAACTAGAAAAATCAAGGCTGCTCAGTGATCGGTTC | |
| | TCTGCAGAGAGGCCTAAGGGATCTTTCTCCACCTTGGAGATCCAGCGCACAGAGCAGGGGGAC | |
| | TCGGCCATGTATCTCTGTGCCAGCAGCTTAACGGTGTCAGATGGCTACACCTTCGGTTCGGGG | |
| | ACCAGGTTAACCGTTGTAGAGGACCTGAACAAGGTGTTCCCACCCGAGGTCGCTGTGTTTGAG | |
| | CCATCAGAAGCAGAGATCTCCCACACCCAAAAGGCCACACTGGTGTGCCTGGCCACAGGCTTC | |
| | TTCCCCGACCACGTGGAGCTGAGCTGGTGGGTGAATGGGAAGGAGGTGCACAGTGGGGTCAGC | |
| | ACGGACCCGCAGCCCCTCAAGGAGCAGCCCGCCCTCAATGACTCCAGATACTGCCTGAGCAGC | |
| | CGCCTGAGGGTCTCGGCCACCTTCTGGCAGAACCCCCGCAACCACTTCCGCTGTCAAGTCCAG | |
| | TTCTACGGGCTCTCGGAGAATGACGAGTGGACCCAGGATAGGGCCAAACCCGTCACCCAGATC | |
| | GTCAGCGCCGAGGCCTGGGGTAGAGCAGACTGTGGCTTTACCTCGGTGTCCTACCAGCAAGGG | |
| | GTCCTGTCTGCCACCATCCTCTATGAGATCCTGCTAGGGAAGGCCACCCTGTATGCTGTGCTG | |
| | GTCAGCGCCCTTGTGTTGATGGCCATGGTCAAGAGAAAGGATTTCTGA | |
| | α chain nucleotides: 1-825 | |
| | P2A nucleotides: 826-882 | |
| | ß chain nucleotides: 883-1812 | |
| 399 | ATAYSGGGADGLT | TCR Y Vα |
| | | CDR-3 |
| 400 | NSQQGEEDPQALSIQEGENATMNCSYKTSINNLQWYRQNSGRGLVHLILIRSNEREKHSGRLR | TCR Y Vα |
| | VTLDTSKKSSSLLITASRAADTASYFCATAYSGGGADGLTFGKGTHLIIQPY | (aa) |
| 401 | METLLGVSLVILWLQLARVNSQQGEEDPQALSIQEGENATMNCSYKTSINNLQWYRQNSGRGL | TCR Y Vα + |
| | VHLILIRSNEREKHSGRLRVTLDTSKKSSSLLITASRAADTASYFCATAYSGGGADGLTFGKG | ss (aa) |
| | THLIIQPY | |
| 402 | METLLGVSLVILWLQLARVNSQQGEEDPQALSIQEGENATMNCSYKTSINNLQWYRQNSGRGL | TCR Y Vα + |
| | VHLILIRSNEREKHSGRLRVTLDTSKKSSSLLITASRAADTASYFCATAYSGGGADGLTFGKG | Cα (aa) |
| | THLIIQPYIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSS | |
| 403 | ASSLIRAEKL | TCR Y Vβ |
| | | CDR-3 |
| 404 | DTGVSQNPRHKITKRGQNVTFRCDPISEHNRLYWYRQTLGQGPEFLTYFQNEAQLEKSRLLSD | TCR Y Vβ |
| | RFSAERPKGSFSTLEIQRTEQGDSAMYLCASSLIRAEKLFFGSGTQLSVLE | (aa) |
| 405 | MGTSLLCWMALCLLGADHADTGVSQNPRHKITKRGQNVTFRCDPISEHNRLYWYRQTLGQGPE | TCR Y Vβ + |
| | FLTYFQNEAQLEKSRLLSDRFSAERPKGSFSTLEIQRTEQGDSAMYLCASSLIRAEKLFFGSG | ss (aa) |
| | TQLSVLE | |
| 406 | MGTSLLCWMALCLLGADHADTGVSQNPRHKITKRGQNVTFRCDPISEHNRLYWYROTLGQGPE | TCR Y Vβ + |
| | FLTYFQNEAQLEKSRLLSDRFSAERPKGSFSTLEIQRTEQGDSAMYLCASSLIRAEKLFFGSG | Cß (aa) |
| | TQLSVLEDLNKVFPPEVAVFEPSEAEISHTQKATLVCLATGFFPDHVELSWWVNGKEVHSGVS | |
| | TDPQPLKEQPALNDSRYCLSSRLRVSATFWQNPRNHERCQVQFYGLSENDEWTQDRAKPVTQI | |
| | VSAEAWGRADCGFTSVSYQQGVLSATILYEILLGKATLYAVLVSALVLMAMVKRKDF | |
| 407 | METLLGVSLVILWLQLARVNSQQGEEDPQALSIQEGENATMNCSYKTSINNLQWYRQNSGRGL | TCR Y |
| | VHLILIRSNEREKHSGRLRVTLDTSKKSSSLLITASRAADTASYFCATAYSGGGADGLTFGKG | protein |
| | THLIIQPYIQNPDPAVYQLRDSKSSDKSVCLFTDFDSQTNVSQSKDSDVYITDKTVLDMRSMD | |
| | FKSNSAVAWSNKSDFACANAFNNSIIPEDTFFPSPESSCDVKLVEKSFETDTNLNFQNLSVIG | |
| | FRILLLKVAGFNLLMTLRLWSSGSGATNFSLLKQAGDVEENPGPMGTSLLCWMALCLLGADHA | |
| | DTGVSQNPRHKITKRGQNVTFRCDPISEHNRLYWYRQTLGQGPEFLTYFQNEAQLEKSRLLSD | |
| | RFSAERPKGSFSTLEIQRTEQGDSAMYLCASSLIRAEKLFFGSGTQLSVLEDLNKVFPPEVAV | |
| | FEPSEAEISHTQKATLVCLATGFFPDHVELSWWVNGKEVHSGVSTDPQPLKEQPALNDSRYCL | |
| | SSRLRVSATFWQNPRNHFRCQVQFYGLSENDEWTQDRAKPVTQIVSAEAWGRADCGFTSVSYQ | |
| | QGVLSATILYEILLGKATLYAVLVSALVLMAMVKRKDF | |
| 408 | ATGGAAACTCTCCTGGGAGTGTCTTTGGTGATTCTATGGCTTCAACTGGCTAGGGTGAACAGT | TCR Y (nt) |
| | CAACAGGGAGAAGAGGATCCTCAGGCCTTGAGCATCCAGGAGGGTGAAAATGCCACCATGAAC | |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACGGCGTATTCAGGAGGAGGTGCTGACGGACTCACCTTTGGCAAAGGG | |
| | ACTCATCTAATCATCCAGCCCTATATCCAGAACCCTGACCCTGCCGTGTACCAGCTGAGAGAC | |
| | TCTAAATCCAGTGACAAGTCTGTCTGCCTATTCACCGATTTTGATTCTCAAACAAATGTGTCA | |
| | CAAAGTAAGGATTCTGATGTGTATATCACAGACAAAACTGTGCTAGACATGAGGTCTATGGAC | |
| | TTCAAGAGCAACAGTGCTGTGGCCTGGAGCAACAAATCTGACTTTGCATGTGCAAACGCCTTC | |
| | AACAACAGCATTATTCCAGAAGACACCTTCTTCCCCAGCCCAGAAAGTTCCTGTGATGTCAAG | |
| | CTGGTCGAGAAAAGCTTTGAAACAGATACGAACCTAAACTTTCAAAACCTGTCAGTGATTGGG | |
| | TTCCGAATCCTCCTCCTGAAAGTGGCCGGGTTTAATCTGCTCATGACGCTGCGGCTGTGGTCC | |
| | AGCGGCTCCGGAGCCACGAACTTCTCTCTGTTAAAGCAAGCAGGAGACGTGGAAGAAAACCCC | |
| | GGTCCCATGGGCACCAGCCTCCTCTGCTGGATGGCCCTGTGTCTCCTGGGGGCAGATCACGCA | |
| | GATACTGGAGTCTCCCAGAACCCCAGACACAAGATCACAAAGAGGGGACAGAATGTAACTTTC | |
| | AGGTGTGATCCAATTTCTGAACACAACCGCCTTTATTGGTACCGACAGACCCTGGGGCAGGGC | |
| | CCAGAGTTTCTGACTTACTTCCAGAATGAAGCTCAACTAGAAAAATCAAGGCTGCTCAGTGAT | |
| | CGGTTCTCTGCAGAGAGGCCTAAGGGATCTTTCTCCACCTTGGAGATCCAGCGCACAGAGCAG | |
| | GGGGACTCGGCCATGTATCTCTGTGCCAGCAGCTTAATTCGTGCGGAAAAACTGTTTTTTGGC | |
| | AGTGGAACCCAGCTCTCTGTCTTGGAGGACCTGAACAAGGTGTTCCCACCCGAGGTCGCTGTG | |
| | TTTGAGCCATCAGAAGCAGAGATCTCCCACACCCAAAAGGCCACACTGGTGTGCCTGGCCACA | |
| | GGCTTCTTCCCCGACCACGTGGAGCTGAGCTGGTGGGTGAATGGGAAGGAGGTGCACAGTGGG | |
| | GTCAGCACGGACCCGCAGCCCCTCAAGGAGCAGCCCGCCCTCAATGACTCCAGATACTGCCTG | |
| | AGCAGCCGCCTGAGGGTCTCGGCCACCTTCTGGCAGAACCCCCGCAACCACTTCCGCTGTCAA | |
| | GTCCAGTTCTACGGGCTCTCGGAGAATGACGAGTGGACCCAGGATAGGGCCAAACCCGTCACC | |
| | CAGATCGTCAGCGCCGAGGCCTGGGGTAGAGCAGACTGTGGCTTTACCTCGGTGTCCTACCAG | |
| | CAAGGGGTCCTGTCTGCCACCATCCTCTATGAGATCCTGCTAGGGAAGGCCACCCTGTATGCT | |
| | GTGCTGGTCAGCGCCCTTGTGTTGATGGCCATGGTCAAGAGAAAGGATTTCTAG | |
| | α chain nucleotides:: 1-831 | |
| | P2A nucleotides: 832-888 | |
| | ß chain nucleotides: 889-1818 | |
| 409 | AGPKGNTPLV | TCR Z Vα |
| | | CDR-3 |
| 410 | QQVMQIPQYQHVQEGEDFTTYCNSSTTLSNIQWYKQRPGGHPVFLIQLVKSGEVKKQKRLTFQ | TCR Z Vα |
| | FGEAKKNSSLHITATQTTDVGTYFCAGPKGNTPLVFGKGTRLSVIAN | (aa) |
| 411 | MLLITSMLVLWMQLSQVNGQQVMQIPQYQHVQEGEDFTTYCNSSTTLSNIQWYKQRPGGHPVF | TCR Z Vα + |
| | LIQLVKSGEVKKQKRLTFQFGEAKKNSSLHITATQTTDVGTYFCAGPKGNTPLVFGKGTRLSV | ss (aa) |
| | IAN | |
| 412 | MLLITSMLVLWMQLSQVNGQQVMQIPQYQHVQEGEDFTTYCNSSTTLSNIQWYKQRPGGHPVF | TCR Z Vα + |
| | LIQLVKSGEVKKQKRLTFQFGEAKKNSSLHITATQTTDVGTYFCAGPKGNTPLVFGKGTRLSV | Cα (aa) |
| | IANIQNPDPAVYQLRDSKSSDKSVCLFTDFDSQTNVSQSKDSDVYITDKTVLDMRSMDFKSNS | |
| | AVAWSNKSDFACANAFNNSIIPEDTFFPSPESSCDVKLVEKSFETDTNLNFQNLSVIGFRILL | |
| | LKVAGFNLLMTLRLWSS | |
| 413 | ASSLTVSYEQY | TCR Z Vβ |
| | | CDR-3 |
| 414 | DTGVSQDPRHKITKRGQNVTFRCDPISEHNRLYWYRQTLGQGPEFLTYFQNEAQLEKSRLLSD | TCR Z Vβ |
| | RFSAERPKGSFSTLEIQRTEQGDSAMYLCASSLTVSYEQYFGPGTRLTVTE | (aa) |
| 415 | MGTSLLCWMALCLLGADHADTGVSQDPRHKITKRGQNVTFRCDPISEHNRLYWYRQTLGQGPE | TCR Z Vβ + |
| | FLTYFQNEAQLEKSRLLSDRFSAERPKGSFSTLEIQRTEQGDSAMYLCASSLTVSYEQYFGPG | ss (aa) |
| | TRLTVTE | |
| 416 | MGTSLLCWMALCLLGADHADTGVSQDPRHKITKRGQNVTFRCDPISEHNRLYWYRQTLGQGPE | TCR Z Vβ + |
| | FLTYFQNEAQLEKSRLLSDRFSAERPKGSFSTLEIQRTEQGDSAMYLCASSLTVSYEQYFGPG | Cß (aa) |
| | TRLTVTEDDLKNVFPPEVAVFEPSEAEISHTQKATLVCLATGFYPDHVELSWWVNGKEVHSGV | |
| | STDPQPLKEQPALNDSRYCLSSRLRVSATFWQNPRNHERCQVQFYGLSENDEWTQDRAKPVTQ | |
| | IVSAEAWGRADCGFTSESYQQGVLSATILYEILLGKATLYAVLVSALVLMAMVKRKDSRG | |
| 417 | MLLITSMLVLWMQLSQVNGQQVMQIPQYQHVQEGEDFTTYCNSSTTLSNIQWYKQRPGGHPVF | TCR Z |
| | LIQLVKSGEVKKQKRLTFQFGEAKKNSSLHITATQTTDVGTYFCAGPKGNTPLVFGKGTRLSV | protein |
| | IANIQNPDPAVYQLRDSKSSDKSVCLFTDFDSQTNVSQSKDSDVYITDKTVLDMRSMDFKSNS | |
| | AVAWSNKSDFACANAFNNSIIPEDTFFPSPESSCDVKLVEKSFETDTNLNFQNLSVIGFRILL | |
| | LKVAGFNLLMTLRLWSSGSGATNFSLLKQAGDVEENPGPMGTSLLCWMALCLLGADHADTGVS | |
| | QDPRHKITKRGQNVTFRCDPISEHNRLYWYRQTLGQGPEFLTYFQNEAQLEKSRLLSDRESAE | |
| | RPKGSFSTLEIQRTEQGDSAMYLCASSLTVSYEQYFGPGTRLTVTEDLKNVEPPEVAVFEPSE | |
| | AEISHTQKATLVCLATGFYPDHVELSWWVNGKEVHSGVSTDPQPLKEQPALNDSRYCLSSRLR | |
| | VSATFWQNPRNHFRCQVQFYGLSENDEWTQDRAKPVTQIVSAEAWGRADCGFTSESYQQGVLS | |
| | ATILYEILLGKATLYAVLVSALVLMAMVKRKDSRG | |
| 418 | ATGCTACTCATCACATCAATGTTGGTCTTATGGATGCAATTGTCACAGGTGAATGGACAACAG | TCR Z (nt) |
| | GTAATGCAAATTCCTCAGTACCAGCATGTACAAGAAGGAGAGGACTTCACCACGTACTGCAAT | |
| | TCCTCAACTACTTTAAGCAATATACAGTGGTATAAGCAAAGGCCTGGTGGACATCCCGTTTTT | |
| | TTGATACAGTTAGTGAAGAGTGGAGAAGTGAAGAAGCAGAAAAGACTGACATTTCAGTTTGGA | |
| | GAAGCAAAAAAGAACAGCTCCCTGCACATCACAGCCACCCAGACTACAGATGTAGGAACCTAC | |
| | TTCTGTGCAGGGCCGAAAGGAAACACACCTCTTGTCTTTGGAAAGGGCACAAGACTTTCTGTG | |
| | ATTGCAAATATCCAGAACCCTGACCCTGCCGTGTACCAGCTGAGAGACTCTAAATCCAGTGAC | |
| | AAGTCTGTCTGCCTATTCACCGATTTTGATTCTCAAACAAATGTGTCACAAAGTAAGGATTCT | |
| | GATGTGTATATCACAGACAAAACTGTGCTAGACATGAGGTCTATGGACTTCAAGAGCAACAGT | |
| | GCTGTGGCCTGGAGCAACAAATCTGACTTTGCATGTGCAAACGCCTTCAACAACAGCATTATT | |
| | CCAGAAGACACCTTCTTCCCCAGCCCAGAAAGTTCCTGTGATGTCAAGCTGGTCGAGAAAAGC | |
| | TTTGAAACAGATACGAACCTAAACTTTCAAAACCTGTCAGTGATTGGGTTCCGAATCCTCCTC | |
| | CTGAAAGTGGCCGGGTTTAATCTGCTCATGACGCTGCGGCTGTGGTCCAGCGGCTCCGGAGCC | |
| | ACGAACTTCTCTCTGTTAAAGCAAGCAGGAGACGTGGAAGAAAACCCCGGTCCCATGGGCACC | |
| | AGCCTCCTCTGCTGGATGGCCCTGTGTCTCCTGGGGGCAGATCACGCAGATACTGGAGTCTCC | |
| | CAGGACCCCAGACACAAGATCACAAAGAGGGGACAGAATGTAACTTTCAGGTGTGATCCAATT | |
| | TCTGAACACAACCGCCTTTATTGGTACCGACAGACCCTGGGGCAGGGCCCAGAGTTTCTGACT | |
| | TACTTCCAGAATGAAGCTCAACTAGAAAAATCAAGGCTGCTCAGTGATCGGTTCTCTGCAGAG | |
| | AGGCCTAAGGGATCTTTCTCCACCTTGGAGATCCAGCGCACAGAGCAGGGGGACTCGGCCATG | |
| | TATCTCTGTGCCAGCAGCTTAACAGTCTCCTACGAGCAGTACTTCGGGCCGGGCACCAGGCTC | |
| | ACGGTCACAGAGGACCTGAAAAACGTGTTCCCACCCGAGGTCGCTGTGTTTGAGCCATCAGAA | |
| | GCAGAGATCTCCCACACCCAAAAGGCCACACTGGTGTGCCTGGCCACAGGCTTCTACCCCGAC | |
| | CACGTGGAGCTGAGCTGGTGGGTGAATGGGAAGGAGGTGCACAGTGGGGTCAGCACAGACCCG | |
| | CAGCCCCTCAAGGAGCAGCCCGCCCTCAATGACTCCAGATACTGCCTGAGCAGCCGCCTGAGG | |
| | GTCTCGGCCACCTTCTGGCAGAACCCCCGCAACCACTTCCGCTGTCAAGTCCAGTTCTACGGG | |
| | CTCTCGGAGAATGACGAGTGGACCCAGGATAGGGCCAAACCTGTCACCCAGATCGTCAGCGCC | |
| | GAGGCCTGGGGTAGAGCAGACTGTGGCTTCACCTCCGAGTCTTACCAGCAAGGGGTCCTGTCT | |
| | GCCACCATCCTCTATGAGATCTTGCTAGGGAAGGCCACCTTGTATGCCGTGCTGGTCAGTGCC | |
| | CTCGTGCTGATGGCCATGGTCAAGAGAAAGGATTCCAGAGGCTGA | |
| | α chain nucleotides:: 1-816 | |
| | P2A nucleotides: 817-873 | |
| | ß chain nucleotides: 874-1809 | |
| 419 | AASSPGTYKYI | TCR AA Vα |
| | | CDR-3 |
| 420 | SGQQKEKSDQQQVKQSPQSLIVQKGGISIINCAYENTAFDYFPWYQQFPGKGPALLIAIRPDV | TCR AA Vα |
| | SEKKEGRFTISFNKSAKQFSLHIMDSQPGDSATYFCAASSPGTYKYIFGTGTRLKVLAN | (aa) |
| 421 | MDKILGASFLVLWLQLCWVSGQQKEKSDQQQVKQSPQSLIVQKGGISIINCAYENTAFDYFPW | TCR AA Vα + |
| | YQQFPGKGPALLIAIRPDVSEKKEGRFTISFNKSAKQFSLHIMDSQPGDSATYFCAASSPGTY | ss (aa) |
| | KYIFGTGTRLKVLAN | |
| 422 | MDKILGASFLVLWLQLCWVSGQQKEKSDQQQVKQSPQSLIVQKGGISIINCAYENTAFDYFPW | TCR AA Vα + |
| | YQQFPGKGPALLIAIRPDVSEKKEGRFTISFNKSAKQFSLHIMDSQPGDSATYFCAASSPGTY | Ca (aa) |
| | KYIFGTGTRLKVLANIQNPDPAVYQLRDSKSSDKSVCLFTDFDSQTNVSQSKDSDVYITDKTV | |
| | LDMRSMDFKSNSAVAWSNKSDFACANAFNNSIIPEDTFFPSPESSCDVKLVEKSFETDTNLNF | |
| | QNLSVIGFRILLLKVAGFNLLMTLRLWSS | |
| 423 | ASSLLKNTEA | TCR AA Vβ |
| | | CDR-3 |
| 424 | DTGVSQDPRHKITKRGQNVTFRCDPISEHNRLYWYRQTLGQGPEFLTYFQNEAQLEKSRLLSD | TCR AA Vβ |
| | RFSAERPKGSFSTLEIQRTEQGDSAMYLCASSLLKNTEAFFGQGTRLTVVE | (aa) |
| 425 | MGTSLLCWMALCLLGADHADTGVSQDPRHKITKRGQNVTFRCDPISEHNRLYWYRQTLGQGPE | TCR AA Vβ + |
| | FLTYFQNEAQLEKSRLLSDRFSAERPKGSFSTLEIQRTEQGDSAMYLCASSLLKNTEAFFGQG | ss (aa) |
| | TRLTVVE | |
| 426 | MGTSLLCWMALCLLGADHADTGVSQDPRHKITKRGQNVTFRCDPISEHNRLYWYRQTLGQGPE | TCR AA Vβ + |
| | FLTYFQNEAQLEKSRLLSDRFSAERPKGSFSTLEIQRTEQGDSAMYLCASSLLKNTEAFFGQG | Cß (aa) |
| | TRLTVVEDLNKVFPPEVAVFEPSEAEISHTQKATLVCLATGFFPDHVELSWWVNGKEVHSGVS | |
| | TDPQPLKEQPALNDSRYCLSSRLRVSATFWQNPRNHERCQVQFYGLSENDEWTQDRAKPVTQI | |
| | VSAEAWGRADCGFTSVSYQQGVLSATILYEILLGKATLYAVLVSALVLMAMVKRKDF | |
| 427 | MDKILGASFLVLWLQLCWVSGQQKEKSDQQQVKQSPQSLIVQKGGISIINCAYENTAFDYFPW | TCR AA |
| | YQQFPGKGPALLIAIRPDVSEKKEGRFTISFNKSAKQFSLHIMDSQPGDSATYFCAASSPGTY | protein |
| | KYIFGTGTRLKVLANIQNPDPAVYQLRDSKSSDKSVCLFTDFDSQTNVSQSKDSDVYITDKTV | |
| | LDMRSMDFKSNSAVAWSNKSDFACANAFNNSIIPEDTFFPSPESSCDVKLVEKSFETDTNLNF | |
| | QNLSVIGFRILLLKVAGENLLMTLRLWSSGSGATNFSLLKQAGDVEENPGPMGTSLLCWMALC | |
| | LLGADHADTGVSQDPRHKITKRGQNVTFRCDPISEHNRLYWYRQTLGQGPEFLTYFQNEAQLE | |
| | KSRLLSDRFSAERPKGSESTLEIQRTEQGDSAMYLCASSLLKNTEAFFGQGTRLTVVEDLNKV | |
| | FPPEVAVFEPSEAEISHTQKATLVCLATGFFPDHVELSWWVNGKEVHSGVSTDPQPLKEQPAL | |
| | NDSRYCLSSRLRVSATFWQNPRNHFRCQVQFYGLSENDEWTQDRAKPVTQIVSAEAWGRADCG | |
| | FTSVSYQQGVLSATILYEILLGKATLYAVLVSALVLMAMVKRKDF | |
| 428 | ATGGACAAGATCTTAGGAGCATCATTTTTAGTTCTGTGGCTTCAACTATGCTGGGTGAGTGGC | TCR AA (nt) |
| | CAACAGAAGGAGAAAAGTGACCAGCAGCAGGTGAAACAAAGTCCTCAATCTTTGATAGTCCAG | |
| | AAAGGAGGGATTTCAATTATAAACTGTGCTTATGAGAACACTGCGTTTGACTACTTTCCATGG | |
| | TACCAACAATTCCCTGGGAAAGGCCCTGCATTATTGATAGCCATACGTCCAGATGTGAGTGAA | |
| | AAGAAAGAAGGAAGATTCACAATCTCCTTCAATAAAAGTGCCAAGCAGTTCTCATTGCATATC | |
| | ATGGATTCCCAGCCTGGAGACTCAGCCACCTACTTCTGTGCAGCAAGCAGCCCAGGAACCTAC | |
| | AAATACATCTTTGGAACAGGCACCAGGCTGAAGGTTTTAGCAAATATCCAGAACCCTGACCCT | |
| | GCCGTGTACCAGCTGAGAGACTCTAAATCCAGTGACAAGTCTGTCTGCCTATTCACCGATTTT | |
| | GATTCTCAAACAAATGTGTCACAAAGTAAGGATTCTGATGTGTATATCACAGACAAAACTGTG | |
| | CTAGACATGAGGTCTATGGACTTCAAGAGCAACAGTGCTGTGGCCTGGAGCAACAAATCTGAC | |
| | TTTGCATGTGCAAACGCCTTCAACAACAGCATTATTCCAGAAGACACCTTCTTCCCCAGCCCA | |
| | GAAAGTTCCTGTGATGTCAAGCTGGTCGAGAAAAGCTTTGAAACAGATACGAACCTAAACTTT | |
| | CAAAACCTGTCAGTGATTGGGTTCCGAATCCTCCTCCTGAAAGTGGCCGGGTTTAATCTGCTC | |
| | ATGACGCTGCGGCTGTGGTCCAGCGGCTCCGGAGCCACGAACTTCTCTCTGTTAAAGCAAGCA | |
| | GGAGACGTGGAAGAAAACCCCGGTCCCATGGGCACCAGCCTCCTCTGCTGGATGGCCCTGTGT | |
| | CTCCTGGGGGCAGATCACGCAGATACTGGAGTCTCCCAGGACCCCAGACACAAGATCACAAAG | |
| | AGGGGACAGAATGTAACTTTCAGGTGTGATCCAATTTCTGAACACAACCGCCTTTATTGGTAC | |
| | CGACAGACCCTGGGGCAGGGCCCAGAGTTTCTGACTTACTTCCAGAATGAAGCTCAACTAGAA | |
| | AAATCAAGGCTGCTCAGTGATCGGTTCTCTGCAGAGAGGCCTAAGGGATCTTTCTCCACCTTG | |
| | GAGATCCAGCGCACAGAGCAGGGGGACTCGGCCATGTATCTCTGTGCCAGCAGCCTCTTGAAG | |
| | AACACTGAAGCTTTCTTTGGACAAGGCACCAGACTCACAGTTGTAGAGGACCTGAACAAGGTG | |
| | TTCCCACCCGAGGTCGCTGTGTTTGAGCCATCAGAAGCAGAGATCTCCCACACCCAAAAGGCC | |
| | ACACTGGTGTGCCTGGCCACAGGCTTCTTCCCCGACCACGTGGAGCTGAGCTGGTGGGTGAAT | |
| | GGGAAGGAGGTGCACAGTGGGGTCAGCACGGACCCGCAGCCCCTCAAGGAGCAGCCCGCCCTC | |
| | AATGACTCCAGATACTGCCTGAGCAGCCGCCTGAGGGTCTCGGCCACCTTCTGGCAGAACCCC | |
| | CGCAACCACTTCCGCTGTCAAGTCCAGTTCTACGGGCTCTCGGAGAATGACGAGTGGACCCAG | |
| | GATAGGGCCAAACCCGTCACCCAGATCGTCAGCGCCGAGGCCTGGGGTAGAGCAGACTGTGGC | |
| | TTTACCTCGGTGTCCTACCAGCAAGGGGTCCTGTCTGCCACCATCCTCTATGAGATCCTGCTA | |
| | GGGAAGGCCACCCTGTATGCTGTGCTGGTCAGCGCCCTTGTGTTGATGGCCATGGTCAAGAGA | |
| | AAGGATTTCTGA | |
| | α chain nucleotides: 1-852 | |
| | P2A nucleotides: 853-909 | |
| | ß chain nucleotides: 910-1839 | |
| 429 | AVRVPTGGYQKVT | TCR AB Vα |
| | | CDR-3 |
| 430 | AQSVAQPEDQVNVAEGNPLTVKCTYSVSGNPYLFWYVQYPNRGLQFLLKYITGDNLVKGSYGF | TCR AB Vα |
| | EAEFNKSQTSFHLKKPSALVSDSALYFCAVRVPTGGYQKVTFGIGTKLQVIPN | (aa) |
| 431 | MASAPISMLAMLFTLSGLRAQSVAQPEDQVNVAEGNPLTVKCTYSVSGNPYLFWYVQYPNRGL | TCR AB Vα + |
| | QFLLKYITGDNLVKGSYGFEAEFNKSQTSFHLKKPSALVSDSALYFCAVRVPTGGYQKVTFGI | ss (aa) |
| | GTKLQVIPN | |
| 432 | MASAPISMLAMLFTLSGLRAQSVAQPEDQVNVAEGNPLTVKCTYSVSGNPYLFWYVQYPNRGL | TCR AB Vα + |
| | QFLLKYITGDNLVKGSYGFEAEFNKSQTSFHLKKPSALVSDSALYFCAVRVPTGGYQKVTFGI | Cα (aa) |
| | GTKLQVIPNIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGFNLLMTLRLWSS | |
| 433 | ASKLTDTQY | TCR AB Vβ |
| | | CDR-3 |
| 434 | DTGVSQNPRHKITKRGQNVTFRCDPISEHNRLYWYRQTLGQGPEFLTYFQNEAQLEKSRLLSD | TCR AB Vβ |
| | RFSAERPKGSFSTLEIQRTEQGDSAMYLCASKLTDTQYFGPGTRLTVLE | (aa) |
| 435 | MGTSLLCWMALCLLGADHADTGVSQNPRHKITKRGQNVTFRCDPISEHNRLYWYRQTLGQGPE | TCR AB Vβ + |
| | FLTYFQNEAQLEKSRLLSDRFSAERPKGSFSTLEIQRTEQGDSAMYLCASKLTDTQYFGPGTR | ss (aa) |
| | LTVLE | |
| 436 | MGTSLLCWMALCLLGADHADTGVSQNPRHKITKRGQNVTFRCDPISEHNRLYWYRQTLGQGPE | TCR AB Vβ + |
| | FLTYFQNEAQLEKSRLLSDRFSAERPKGSFSTLEIQRTEQGDSAMYLCASKLTDTQYFGPGTR | Cß (aa) |
| | LTVLEDLKNVFPPEVAVFEPSEAEISHTQKATLVCLATGFYPDHVELSWWVNGKEVHSGVSTD | |
| | PQPLKEQPALNDSRYCLSSRLRVSATFWQNPRNHERCQVQFYGLSENDEWTQDRAKPVTQIVS | |
| | AEAWGRADCGFTSESYQQGVLSATILYEILLGKATLYAVLVSALVLMAMVKRKDSRG | |
| 437 | MASAPISMLAMLFTLSGLRAQSVAQPEDQVNVAEGNPLTVKCTYSVSGNPYLFWYVQYPNRGL | TCR AB |
| | QFLLKYITGDNLVKGSYGFEAEFNKSQTSFHLKKPSALVSDSALYFCAVRVPTGGYQKVTFGI | protein |
| | GTKLQVIPNIQNPDPAVYQLRDSKSSDKSVCLFTDFDSQTNVSQSKDSDVYITDKTVLDMRSM | |
| | DFKSNSAVAWSNKSDFACANAFNNSIIPEDTFFPSPESSCDVKLVEKSFETDTNLNFQNLSVI | |
| | GFRILLLKVAGENLLMTLRLWSSGSGATNFSLLKQAGDVEENPGPMGTSLLCWMALCLLGADH | |
| | ADTGVSQNPRHKITKRGQNVTFRCDPISEHNRLYWYRQTLGQGPEFLTYFQNEAQLEKSRLLS | |
| | DRFSAERPKGSFSTLEIQRTEQGDSAMYLCASKLTDTQYFGPGTRLTVLEDLKNVFPPEVAVE | |
| | EPSEAEISHTQKATLVCLATGFYPDHVELSWWVNGKEVHSGVSTDPQPLKEQPALNDSRYCLS | |
| | SRLRVSATFWQNPRNHFRCQVQFYGLSENDEWTQDRAKPVTQIVSAEAWGRADCGFTSESYQQ | |
| | GVLSATILYEILLGKATLYAVLVSALVLMAMVKRKDSRG | |
| 438 | ATGGCCTCTGCACCCATCTCGATGCTTGCGATGCTCTTCACATTGAGTGGGCTGAGAGCTCAG | TCR AB (nt) |
| | TCAGTGGCTCAGCCGGAAGATCAGGTCAACGTTGCTGAAGGGAATCCTCTGACTGTGAAATGC | |
| | ACCTATTCAGTCTCTGGAAACCCTTATCTTTTTTGGTATGTTCAATACCCCAACCGAGGCCTC | |
| | CAGTTCCTTCTGAAATACATCACAGGGGATAACCTGGTTAAAGGCAGCTATGGCTTTGAAGCT | |
| | GAATTTAACAAGAGCCAAACCTCCTTCCACCTGAAGAAACCATCTGCCCTTGTGAGCGACTCC | |
| | GCTTTGTACTTCTGTGCTGTGAGAGTCCCGACTGGGGGTTACCAGAAAGTTACCTTTGGAATT | |
| | GGAACAAAGCTCCAAGTCATCCCAAATATCCAGAACCCTGACCCTGCCGTGTACCAGCTGAGA | |
| | GACTCTAAATCCAGTGACAAGTCTGTCTGCCTATTCACCGATTTTGATTCTCAAACAAATGTG | |
| | TCACAAAGTAAGGATTCTGATGTGTATATCACAGACAAAACTGTGCTAGACATGAGGTCTATG | |
| | GACTTCAAGAGCAACAGTGCTGTGGCCTGGAGCAACAAATCTGACTTTGCATGTGCAAACGCC | |
| | TTCAACAACAGCATTATTCCAGAAGACACCTTCTTCCCCAGCCCAGAAAGTTCCTGTGATGTC | |
| | AAGCTGGTCGAGAAAAGCTTTGAAACAGATACGAACCTAAACTTTCAAAACCTGTCAGTGATT | |
| | GGGTTCCGAATCCTCCTCCTGAAAGTGGCCGGGTTTAATCTGCTCATGACGCTGCGGCTGTGG | |
| | TCCAGCGGCTCCGGAGCCACGAACTTCTCTCTGTTAAAGCAAGCAGGAGACGTGGAAGAAAAC | |
| | CCCGGTCCCATGGGCACCAGCCTCCTCTGCTGGATGGCCCTGTGTCTCCTGGGGGCAGATCAC | |
| | GCAGATACTGGAGTCTCCCAGAACCCCAGACACAAGATCACAAAGAGGGGACAGAATGTAACT | |
| | TTCAGGTGTGATCCAATTTCTGAACACAACCGCCTTTATTGGTACCGACAGACCCTGGGGCAG | |
| | GGCCCAGAGTTTCTGACTTACTTCCAGAATGAAGCTCAACTAGAAAAATCAAGGCTGCTCAGT | |
| | GATCGGTTCTCTGCAGAGAGGCCTAAGGGATCTTTCTCCACCTTGGAGATCCAGCGCACAGAG | |
| | CAGGGGGACTCGGCCATGTATCTCTGTGCCAGCAAGCTTACAGATACGCAGTATTTTGGCCCA | |
| | GGCACCCGGCTGACAGTGCTCGAGGACCTGAAAAACGTGTTCCCACCCGAGGTCGCTGTGTTT | |
| | GAGCCATCAGAAGCAGAGATCTCCCACACCCAAAAGGCCACACTGGTGTGCCTGGCCACAGGC | |
| | TTCTACCCCGACCACGTGGAGCTGAGCTGGTGGGTGAATGGGAAGGAGGTGCACAGTGGGGTC | |
| | AGCACAGACCCGCAGCCCCTCAAGGAGCAGCCCGCCCTCAATGACTCCAGATACTGCCTGAGC | |
| | AGCCGCCTGAGGGTCTCGGCCACCTTCTGGCAGAACCCCCGCAACCACTTCCGCTGTCAAGTC | |
| | CAGTTCTACGGGCTCTCGGAGAATGACGAGTGGACCCAGGATAGGGCCAAACCTGTCACCCAG | |
| | ATCGTCAGCGCCGAGGCCTGGGGTAGAGCAGACTGTGGCTTCACCTCCGAGTCTTACCAGCAA | |
| | GGGGTCCTGTCTGCCACCATCCTCTATGAGATCTTGCTAGGGAAGGCCACCTTGTATGCCGTG | |
| | CTGGTCAGTGCCCTCGTGCTGATGGCCATGGTCAAGAGAAAGGATTCCAGAGGCTGA | |
| | α chain nucleotides: : 1-834 | |
| | P2A nucleotides: 835-891 | |
| | ß chain nucleotides: 892-1821 | |
| 439 | AKNDMR | TCR AC Vα |
| | | CDR-3 |
| 440 | NSQQGEEDPQALSIQEGENATMNCSYKTSINNLQWYRQNSGRGLVHLILIRSNEREKHSGRLR | TCR AC Vα |
| | VTLDTSKKSSSLLITASRAADTASYFCAKNDMRFGAGTRLTVKPN | (aa) |
| 441 | METLLGVSLVILWLQLARVNSQQGEEDPQALSIQEGENATMNCSYKTSINNLQWYRQNSGRGL | TCR AC Vα + |
| | VHLILIRSNEREKHSGRLRVTLDTSKKSSSLLITASRAADTASYFCAKNDMRFGAGTRLTVKP | ss (aa) |
| | N | |
| 442 | METLLGVSLVILWLQLARVNSQQGEEDPQALSIQEGENATMNCSYKTSINNLQWYRQNSGRGL | TCR AC Vα + |
| | VHLILIRSNEREKHSGRLRVTLDTSKKSSSLLITASRAADTASYFCAKNDMRFGAGTRLTVKP | Cα (aa) |
| | NIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLK | |
| | VAGFNLLMTLRLWSS | |
| 443 | ASSLLVGETQY | TCR AC Vβ |
| | | CDR-3 |
| 444 | DTGVSQNPRHKITKRGQNVTFRCDPISEHNRLYWYRQTLGQGPEFLTYFQNEAQLEKSRLLSD | TCR AC Vβ |
| | RFSAERPKGSFSTLEIQRTEQGDSAMYLCASSLLVGETQYFGPGTRLLVLE | (aa) |
| 445 | MGTSLLCWMALCLLGADHADTGVSQNPRHKITKRGQNVTFRCDPISEHNRLYWYRQTLGQGPE | TCR AC Vβ + |
| | FLTYFQNEAQLEKSRLLSDRFSAERPKGSFSTLEIQRTEQGDSAMYLCASSLLVGETQYFGPG | ss (aa) |
| | TRLLVLE | |
| 446 | MGTSLLCWMALCLLGADHADTGVSQNPRHKITKRGQNVTFRCDPISEHNRLYWYRQTLGQGPE | TCR AC Vβ + |
| | FLTYFQNEAQLEKSRLLSDRFSAERPKGSESTLEIQRTEQGDSAMYLCASSLLVGETQYFGPG | Cß (aa) |
| | TRLLVLEDLKNVFPPEVAVFEPSEAEISHTQKATLVCLATGFYPDHVELSWWVNGKEVHSGVS | |
| | TDPQPLKEQPALNDSRYCLSSRLRVSATFWQNPRNHERCQVQFYGLSENDEWTQDRAKPVTQI | |
| | VSAEAWGRADCGFTSESYQQGVLSATILYEILLGKATLYAVLVSALVLMAMVKRKDSRG | |
| 447 | METLLGVSLVILWLQLARVNSQQGEEDPQALSIQEGENATMNCSYKTSINNLQWYRQNSGRGL | TCR AC |
| | VHLILIRSNEREKHSGRLRVTLDTSKKSSSLLITASRAADTASYFCAKNDMRFGAGTRLTVKP | protein |
| | NIQNPDPAVYQLRDSKSSDKSVCLFTDFDSQTNVSQSKDSDVYITDKTVLDMRSMDFKSNSAV | |
| | AWSNKSDFACANAFNNSIIPEDTFFPSPESSCDVKLVEKSFETDTNLNFQNLSVIGFRILLLK | |
| | VAGFNLLMTLRLWSSGSGATNFSLLKQAGDVEENPGPMGTSLLCWMALCLLGADHADTGVSQN | |
| | PRHKITKRGQNVTFRCDPISEHNRLYWYRQTLGQGPEFLTYFQNEAQLEKSRLLSDRESAERP | |
| | KGSFSTLEIQRTEQGDSAMYLCASSLLVGETQYFGPGTRLLVLEDLKNVFPPEVAVFEPSEAE | |
| | ISHTQKATLVCLATGFYPDHVELSWWVNGKEVHSGVSTDPQPLKEQPALNDSRYCLSSRLRVS | |
| | ATFWQNPRNHFRCQVQFYGLSENDEWTQDRAKPVTQIVSAEAWGRADCGFTSESYQQGVLSAT | |
| | ILYEILLGKATLYAVLVSALVLMAMVKRKDSRG | |
| 448 | ATGGAAACTCTCCTGGGAGTGTCTTTGGTGATTCTATGGCTTCAACTGGCTAGGGTGAACAGT | TCR AC (nt) |
| | CAACAGGGAGAAGAGGATCCTCAGGCCTTGAGCATCCAGGAGGGTGAAAATGCCACCATGAAC | |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTAAGAATGACATGCGCTTTGGAGCAGGGACCAGACTGACAGTAAAACCA | |
| | AATATCCAGAACCCTGACCCTGCCGTGTACCAGCTGAGAGACTCTAAATCCAGTGACAAGTCT | |
| | GTCTGCCTATTCACCGATTTTGATTCTCAAACAAATGTGTCACAAAGTAAGGATTCTGATGTG | |
| | TATATCACAGACAAAACTGTGCTAGACATGAGGTCTATGGACTTCAAGAGCAACAGTGCTGTG | |
| | GCCTGGAGCAACAAATCTGACTTTGCATGTGCAAACGCCTTCAACAACAGCATTATTCCAGAA | |
| | GACACCTTCTTCCCCAGCCCAGAAAGTTCCTGTGATGTCAAGCTGGTCGAGAAAAGCTTTGAA | |
| | ACAGATACGAACCTAAACTTTCAAAACCTGTCAGTGATTGGGTTCCGAATCCTCCTCCTGAAA | |
| | GTGGCCGGGTTTAATCTGCTCATGACGCTGCGGCTGTGGTCCAGCGGCTCCGGAGCCACGAAC | |
| | TTCTCTCTGTTAAAGCAAGCAGGAGACGTGGAAGAAAACCCCGGTCCCATGGGCACCAGCCTC | |
| | CTCTGCTGGATGGCCCTGTGTCTCCTGGGGGCAGATCACGCAGATACTGGAGTCTCCCAGAAC | |
| | CCCAGACACAAGATCACAAAGAGGGGACAGAATGTAACTTTCAGGTGTGATCCAATTTCTGAA | |
| | CACAACCGCCTTTATTGGTACCGACAGACCCTGGGGCAGGGCCCAGAGTTTCTGACTTACTTC | |
| | CAGAATGAAGCTCAACTAGAAAAATCAAGGCTGCTCAGTGATCGGTTCTCTGCAGAGAGGCCT | |
| | AAGGGATCTTTCTCCACCTTGGAGATCCAGCGCACAGAGCAGGGGGACTCGGCCATGTATCTC | |
| | TGTGCCAGCAGCTTACTGGTGGGAGAGACCCAGTACTTCGGGCCAGGCACGCGGCTCCTGGTG | |
| | CTCGAGGACCTGAAAAACGTGTTCCCACCCGAGGTCGCTGTGTTTGAGCCATCAGAAGCAGAG | |
| | ATCTCCCACACCCAAAAGGCCACACTGGTGTGCCTGGCCACAGGCTTCTACCCCGACCACGTG | |
| | GAGCTGAGCTGGTGGGTGAATGGGAAGGAGGTGCACAGTGGGGTCAGCACAGACCCGCAGCCC | |
| | CTCAAGGAGCAGCCCGCCCTCAATGACTCCAGATACTGCCTGAGCAGCCGCCTGAGGGTCTCG | |
| | GCCACCTTCTGGCAGAACCCCCGCAACCACTTCCGCTGTCAAGTCCAGTTCTACGGGCTCTCG | |
| | GAGAATGACGAGTGGACCCAGGATAGGGCCAAACCTGTCACCCAGATCGTCAGCGCCGAGGCC | |
| | TGGGGTAGAGCAGACTGTGGCTTCACCTCCGAGTCTTACCAGCAAGGGGTCCTGTCTGCCACC | |
| | ATCCTCTATGAGATCTTGCTAGGGAAGGCCACCTTGTATGCCGTGCTGGTCAGTGCCCTCGTG | |
| | CTGATGGCCATGGTCAAGAGAAAGGATTCCAGAGGCTGA | |
| | α chain nucleotides: 1-810 | |
| | P2A nucleotides: 811-867 | |
| | ß chain nucleotides: 868-1803 | |
| 449 | ATVPTDYMYSGGGADGLT | TCR AD Vα |
| | | CDR-3 |
| 450 | NSQQGEEDPQALSIQEGENATMNCSYKTSINNLQWYRQNSGRGLVHLILIRSNEREKHSGRLR | TCR AD Vα |
| | VTLDTSKKSSSLLITASRAADTASYFCATVPTDYMYSGGGADGLTFGKGTHLIIQPY | (aa) |
| 451 | METLLGVSLVILWLQLARVNSQQGEEDPQALSIQEGENATMNCSYKTSINNLQWYRQNSGRGL | TCR AD Vα + |
| | VHLILIRSNEREKHSGRLRVTLDTSKKSSSLLITASRAADTASYFCATVPTDYMYSGGGADGL | ss (aa) |
| | TFGKGTHLIIQPY | |
| 452 | METLLGVSLVILWLQLARVNSQQGEEDPQALSIQEGENATMNCSYKTSINNLQWYRQNSGRGL | TCR AD Vα + |
| | VHLILIRSNEREKHSGRLRVTLDTSKKSSSLLITASRAADTASYFCATVPTDYMYSGGGADGL | Cα (aa) |
| | TFGKGTHLIIQPYIQNPDPAVYQLRDSKSSDKSVCLFTDFDSQTNVSQSKDSDVYITDKTVLD | |
| | MRSMDFKSNSAVAWSNKSDFACANAFNNSIIPEDTFFPSPESSCDVKLVEKSFETDTNLNFQN | |
| | LSVIGFRILLLKVAGFNLLMTLRLWSS | |
| 453 | ASSLVRLDGYT | TCR AD Vβ |
| | | CDR-3 |
| 454 | DTGVSQNPRHKITKRGQNVTFRCDPISEHNRLYWYRQTLGQGPEFLTYFQNEAQLEKSRLLSD | TCR AD Vβ |
| | RFSAERPKGSFSTLEIQRTEQGDSAMYLCASSLVRLDGYTFGSGTRLTVVE | (aa) |
| 455 | MGTSLLCWMALCLLGADHADTGVSQNPRHKITKRGQNVTFRCDPISEHNRLYWYRQTLGQGPE | TCR AD Vβ + |
| | FLTYFQNEAQLEKSRLLSDRFSAERPKGSESTLEIQRTEQGDSAMYLCASSLVRLDGYTFGSG | ss (aa) |
| | TRLTVVE | |
| 456 | MGTSLLCWMALCLLGADHADTGVSQNPRHKITKRGQNVTFRCDPISEHNRLYWYRQTLGQGPE | TCR AD Vβ + |
| | FLTYFQNEAQLEKSRLLSDRFSAERPKGSFSTLEIQRTEQGDSAMYLCASSLVRLDGYTFGSG | Cß (aa) |
| | TRLTVVEDLNKVFPPEVAVFEPSEAEISHTQKATLVCLATGFFPDHVELSWWVNGKEVHSGVS | |
| | TDPQPLKEQPALNDSRYCLSSRLRVSATFWQNPRNHERCQVQFYGLSENDEWTQDRAKPVTQI | |
| | VSAEAWGRADCGFTSVSYQQGVLSATILYEILLGKATLYAVLVSALVLMAMVKRKDF | |
| 457 | METLLGVSLVILWLQLARVNSQQGEEDPQALSIQEGENATMNCSYKTSINNLQWYRQNSGRGL | TCR AD |
| | VHLILIRSNEREKHSGRLRVTLDTSKKSSSLLITASRAADTASYFCATVPTDYMYSGGGADGL | protein |
| | TFGKGTHLIIQPYIQNPDPAVYQLRDSKSSDKSVCLFTDFDSQTNVSQSKDSDVYITDKTVLD | |
| | MRSMDFKSNSAVAWSNKSDFACANAFNNSIIPEDTFFPSPESSCDVKLVEKSFETDTNLNFQN | |
| | LSVIGFRILLLKVAGFNLLMTLRLWSSGSGATNFSLLKQAGDVEENPGPMGTSLLCWMALCLL | |
| | GADHADTGVSQNPRHKITKRGQNVTFRCDPISEHNRLYWYRQTLGQGPEFLTYFQNEAQLEKS | |
| | RLLSDRFSAERPKGSFSTLEIQRTEQGDSAMYLCASSLVRLDGYTFGSGTRLTVVEDLNKVFP | |
| | PEVAVFEPSEAEISHTQKATLVCLATGFFPDHVELSWWVNGKEVHSGVSTDPQPLKEQPALND | |
| | SRYCLSSRLRVSATFWQNPRNHFRCQVQFYGLSENDEWTQDRAKPVTQIVSAEAWGRADCGFT | |
| | SVSYQQGVLSATILYEILLGKATLYAVLVSALVLMAMVKRKDF | |
| 458 | ATGGAAACTCTCCTGGGAGTGTCTTTGGTGATTCTATGGCTTCAACTGGCTAGGGTGAACAGT | TCR AD (nt) |
| | CAACAGGGAGAAGAGGATCCTCAGGCCTTGAGCATCCAGGAGGGTGAAAATGCCACCATGAAC | |
| | TGCAGTTACAAAACTAGTATAAACAATTTACAGTGGTATAGACAAAATTCAGGTAGAGGCCTT | |
| | GTCCACCTAATTTTAATACGTTCAAATGAAAGAGAGAAACACAGTGGAAGATTAAGAGTCACG | |
| | CTTGACACTTCCAAGAAAAGCAGTTCCTTGTTGATCACGGCTTCCCGGGCAGCAGACACTGCT | |
| | TCTTACTTCTGTGCTACGGTCCCGACTGACTACATGTATTCAGGAGGAGGTGCTGACGGACTC | |
| | ACCTTTGGCAAAGGGACTCATCTAATCATCCAGCCCTATATCCAGAACCCTGACCCTGCCGTG | |
| | TACCAGCTGAGAGACTCTAAATCCAGTGACAAGTCTGTCTGCCTATTCACCGATTTTGATTCT | |
| | CAAACAAATGTGTCACAAAGTAAGGATTCTGATGTGTATATCACAGACAAAACTGTGCTAGAC | |
| | ATGAGGTCTATGGACTTCAAGAGCAACAGTGCTGTGGCCTGGAGCAACAAATCTGACTTTGCA | |
| | TGTGCAAACGCCTTCAACAACAGCATTATTCCAGAAGACACCTTCTTCCCCAGCCCAGAAAGT | |
| | TCCTGTGATGTCAAGCTGGTCGAGAAAAGCTTTGAAACAGATACGAACCTAAACTTTCAAAAC | |
| | CTGTCAGTGATTGGGTTCCGAATCCTCCTCCTGAAAGTGGCCGGGTTTAATCTGCTCATGACG | |
| | CTGCGGCTGTGGTCCAGCGGCTCCGGAGCCACGAACTTCTCTCTGTTAAAGCAAGCAGGAGAC | |
| | GTGGAAGAAAACCCCGGTCCCATGGGCACCAGCCTCCTCTGCTGGATGGCCCTGTGTCTCCTG | |
| | GGGGCAGATCACGCAGATACTGGAGTCTCCCAGAACCCCAGACACAAGATCACAAAGAGGGGA | |
| | CAGAATGTAACTTTCAGGTGTGATCCAATTTCTGAACACAACCGCCTTTATTGGTACCGACAG | |
| | ACCCTGGGGCAGGGCCCAGAGTTTCTGACTTACTTCCAGAATGAAGCTCAACTAGAAAAATCA | |
| | AGGCTGCTCAGTGATCGGTTCTCTGCAGAGAGGCCTAAGGGATCTTTCTCCACCTTGGAGATC | |
| | CAGCGCACAGAGCAGGGGGACTCGGCCATGTATCTCTGTGCCAGCAGCTTAGTACGGTTGGAT | |
| | GGCTACACCTTCGGTTCGGGGACCAGGTTAACCGTTGTAGAGGACCTGAACAAGGTGTTCCCA | |
| | CCCGAGGTCGCTGTGTTTGAGCCATCAGAAGCAGAGATCTCCCACACCCAAAAGGCCACACTG | |
| | GTGTGCCTGGCCACAGGCTTCTTCCCCGACCACGTGGAGCTGAGCTGGTGGGTGAATGGGAAG | |
| | GAGGTGCACAGTGGGGTCAGCACGGACCCGCAGCCCCTCAAGGAGCAGCCCGCCCTCAATGAC | |
| | TCCAGATACTGCCTGAGCAGCCGCCTGAGGGTCTCGGCCACCTTCTGGCAGAACCCCCGCAAC | |
| | CACTTCCGCTGTCAAGTCCAGTTCTACGGGCTCTCGGAGAATGACGAGTGGACCCAGGATAGG | |
| | GCCAAACCCGTCACCCAGATCGTCAGCGCCGAGGCCTGGGGTAGAGCAGACTGTGGCTTTACC | |
| | TCGGTGTCCTACCAGCAAGGGGTCCTGTCTGCCACCATCCTCTATGAGATCCTGCTAGGGAAG | |
| | GCCACCCTGTATGCTGTGCTGGTCAGCGCCCTTGTGTTGATGGCCATGGTCAAGAGAAAGGAT | |
| | TTCTGA | |
| | α chain nucleotides: 1-846 | |
| | P2A nucleotides: 847-903 | |
| | ß chain nucleotides: 904-1833 | |
| 459 | AVRX₁X₂TSGTYKYI | TCR Vα CDR- |
| | X₁ is any amino acid | 3 |
| | X₂ is P, S, or T | |
| 460 | AVRX₃X₂TSGTYKYI | TCR Vα CDR- |
| | X₃ is D, E, S, T, N, Q, C, G, P, A, V, I, L, M, F, Y, or W X₂ | 3 |
| | is P, S, or T | |
| 461 | AVRX₄X₂TSGTYKYI | TCR Vα CDR- |
| | X₄ is G, A, C, S, T, V, D, or E | 3 |
| | X₂ is P, S, or T | |
| 462 | AVRGX₂TSGTYKYI | TCR Vα CDR- |
| | X₂ is P, S, or T | 3 |
| 463 | AVRX₁PTSGTYKYI | TCR Vα CDR- |
| | X₄ is G, A, C, S, T, V, D, or E | 3 |
| 464 | AVRGPTSGTYKYI | TCR Vα CDR- |
| | (see also SEQ ID NO: 65) | 3 |
| 465 | ASSX₅X₆X₇GX₈X₉QX₁₀ | TCR Vβ CDR- |
| | X₅ is L or F | 3 |
| | X₆ is V or L | |
| | X₇ is S, Q, N, or A | |
| | X₈ is N or E | |
| | X₉ is E or T | |
| | X₁₀ is F or Y | |
| 466 | ASSLVX₇GX₈X₉QX₁₀ | TCR Vβ CDR- |
| | X₇ is S, Q, N, or A X₈ is N or E | 3 |
| | X₉ is E or T | |
| | X₁₀ is F or Y | |
| 467 | ASSLVX₁₁GNEQF | TCR Vβ CDR- |
| | X₁₁ is S, Q, or N | 3 |
| 468 | ASSX₅X₆AGETQYF | TCR Vβ CDR- |
| | X₅ is L or F | 3 |
| | X₆ is V or L | |
| 469 | ASSLVSGNEQF | TCR Vβ CDR- |
| | | 3 |
| 470 | AVRCPTSGTYKYI | TCR AE Vα |
| | (Ala to Cys mutation at position 4 of the CDR3 sequence SEQ ID | CDR-3 |
| | NO: 51 and corresponding sequences SEQ ID NOs: 52, 53, 54, and 64) | |
| 471 | AVRSPTSGTYKYI | TCR AF Vα |
| | (Ala to Ser mutation at position 4 of the CDR3 sequence SEQ ID | CDR-3 |
| | NO: 51 and corresponding sequences SEQ ID NOs: 52, 53, 54, and 64) | |
| 472 | AVRTPTSGTYKYI | TCR AG Vα |
| | (Ala to Tyr mutation at position 4 of the CDR3 sequence SEQ I | CDR-3 |
| | NO: 51 and corresponding sequences SEQ ID NOs: 52, 53, 54, and 64) | |
| 473 | AVRVPTSGTYKYI | TCR AH Vα |
| | (Ala to Val mutation at position 4 of the CDR3 sequence SEQ ID | CDR-3 |
| | NO: 51 and corresponding sequences SEQ ID NOs: 52, 53, 54, and 64) | |
| 474 | AVREPTSGTYKYI | TCR AI Vα |
| | (Ala to Glu mutation at position 4 of the Vα CDR3 sequence SEQ ID | CDR-3 |
| | NO: 51 and corresponding SEQ ID NOs: 52, 53, 54, and 64) | |
| 475 | ASSLVNGNEQF | TCR AI Vβ |
| | (Ser to Asn mutation at position 6 of the Vβ CDR3 sequence of SEQ | CDR-3 |
| | ID NO: 57 and corresponding SEQ ID NO: 58, 59, 60, and 64) | |
| 476 | AVRX₁X₁TSGTYKYI | TCR Vα CDR- |
| | each X₁ is independently any amino acid | 3 |
| 477 | AVRX₁₂X₁₃TSGTYKYI | TCR Vα CDR- |
| | X₁₂ is A, I, L, V, P, G, C, M, S, T, D, or E | 3 |
| | X₁₃ is S, T, or P | |
| 478 | ASSX₁X₁X₁GX₁X₁QF | TCR Vβ CDR- |
| | each X₁ is independently any amino acid | 3 |
| 479 | ASSX₁₄X₁₅X₁₆GX₁₇X₁₈QF | TCR Vβ CDR- |
| | X₁₄ is A, I, L, V, P, G, F, Y, or W | 3 |
| | X₁₅ is A, I, L, V, P, or G | |
| | X₁₆ is S, T, A, I, L, V, P, G, N, or Q | |
| | X₁₇ is D, E, N, or Q | |
| | X₁₈ is D, E, S, or T | |
| 480 | AVHSNAGGTSYGKLT | TCR AJ Vα |
| | | CDR-3 |
| 481 | SGEDQVTQSPEALRLQEGESSSLNCSYTVSGLRGLFWYRQDPGKGPEFLFTLYSAGEEKEKER | TCR AI Vα |
| | LKATLTKKESFLHITAPKPEDSATYLCAVHSNAGGTSYGKLTFGQGTILTVHPN | (aa) |
| 482 | MEKMLECAFIVLWLQLGWLSGEDQVTQSPEALRLQEGESSSLNCSYTVSGLRGLFWYRQDPGK | TCR AJ Vα + |
| | GPEFLFTLYSAGEEKEKERLKATLTKKESFLHITAPKPEDSATYLCAVHSNAGGTSYGKLTFG | ss (aa) |
| | QGTILTVHPN | |
| 483 | MEKMLECAFIVLWLQLGWLSGEDQVTQSPEALRLQEGESSSLNCSYTVSGLRGLFWYRQDPGK | TCR AJ Vα + |
| | GPEFLFTLYSAGEEKEKERLKATLTKKESFLHITAPKPEDSATYLCAVHSNAGGTSYGKLTFG | Ca (aa) |
| | QGTILTVHPNIQNPDPAVYQLRDSKSSDKSVCLFTDFDSQTNVSQSKDSDVYITDKTVLDMRS | |
| | MDFKSNSAVAWSNKSDFACANAFNNSIIPEDTFFPSPESSCDVKLVEKSFETDTNLNFQNLSV | |
| | IGFRILLLKVAGENLLMTLRLWSS | |
| 484 | ASSADRGSPLH | TCR AJ Vβ |
| | | CDR-3 |
| 485 | DAGVTQSPTHLIKTRGQHVTLRCSPISGHKSVSWYQQVLGQGPQFIFQYYEKEERGRGNFPDR | TCR AJ Vβ |
| | FSARQFPNYSSELNVNALLLGDSALYLCASSADRGSPLHFGNGTRLTVTE | (aa) |
| 486 | MGPGLLCWVLLCLLGAGPVDAGVTQSPTHLIKTRGQHVTLRCSPISGHKSVSWYQQVLGQGPQ | TCR AJ Vβ + |
| | FIFQYYEKEERGRGNFPDRFSARQFPNYSSELNVNALLLGDSALYLCASSADRGSPLHFGNGT | ss (aa) |
| | RLTVTE | |
| 487 | MGPGLLCWVLLCLLGAGPVDAGVTQSPTHLIKTRGQHVTLRCSPISGHKSVSWYQQVLGQGPQ | TCR AJ Vβ + |
| | FIFQYYEKEERGRGNFPDRFSARQFPNYSSELNVNALLLGDSALYLCASSADRGSPLHFGNGT | Cß (aa) |
| | RLTVTEDLNKVFPPEVAVFEPSEAEISHTQKATLVCLATGFFPDHVELSWWVNGKEVHSGVST | |
| | DPQPLKEQPALNDSRYCLSSRLRVSATFWONPRNHFRCQVQFYGLSENDEWTQDRAKPVTQIV | |
| | SAEAWGRADCGFTSVSYQQGVLSATILYEILLGKATLYAVLVSALVLMAMVKRKDF | |
| 488 | MEKMLECAFIVLWLQLGWLSGEDQVTQSPEALRLQEGESSSLNCSYTVSGLRGLFWYRQDPGK | TCR AJ |
| | GPEFLFTLYSAGEEKEKERLKATLTKKESFLHITAPKPEDSATYLCAVHSNAGGTSYGKLTFG | protein |
| | QGTILTVHPNIQNPDPAVYQLRDSKSSDKSVCLFTDFDSQTNVSQSKDSDVYITDKTVLDMRS | |
| | MDFKSNSAVAWSNKSDFACANAFNNSIIPEDTFFPSPESSCDVKLVEKSFETDTNLNFQNLSV | |
| | IGFRILLLKVAGFNLLMTLRLWSSGSGATNFSLLKQAGDVEENPGPMGPGLLCWVLLCLLGAG | |
| | PVDAGVTQSPTHLIKTRGQHVTLRCSPISGHKSVSWYQQVLGQGPQFIFQYYEKEERGRGNFP | |
| | DRFSARQFPNYSSELNVNALLLGDSALYLCASSADRGSPLHFGNGTRLTVTEDLNKVFPPEVA | |
| | VFEPSEAEISHTQKATLVCLATGFFPDHVELSWWVNGKEVHSGVSTDPQPLKEQPALNDSRYC | |
| | LSSRLRVSATFWQNPRNHFRCQVQFYGLSENDEWTQDRAKPVTQIVSAEAWGRADCGFTSVSY | |
| | QQGVLSATILYEILLGKATLYAVLVSALVLMAMVKRKDF | |
| 489 | ATGGAGAAAATGTTGGAGTGTGCATTCATAGTCTTGTGGCTTCAGCTTGGCTGGTTGAGTGGA | TCR AJ (nt) |
| | GAAGACCAGGTGACGCAGAGTCCCGAGGCCCTGAGACTCCAGGAGGGAGAGAGTAGCAGTCTC | |
| | AACTGCAGTTACACAGTCAGCGGTTTAAGAGGGCTGTTCTGGTATAGGCAAGATCCTGGGAAA | |
| | GGCCCTGAATTCCTCTTCACCCTGTATTCAGCTGGGGAAGAAAAGGAGAAAGAAAGGCTAAAA | |
| | GCCACATTAACAAAGAAGGAAAGCTTTCTGCACATCACAGCCCCTAAACCTGAAGACTCAGCC | |
| | ACTTATCTCTGTGCTGTGCATTCTAATGCTGGTGGTACTAGCTATGGAAAGCTGACATTTGGA | |
| | CAAGGGACCATCTTGACTGTCCATCCAAATATCCAGAACCCTGACCCTGCCGTGTACCAGCTG | |
| | AGAGACTCTAAATCCAGTGACAAGTCTGTCTGCCTATTCACCGATTTTGATTCTCAAACAAAT | |
| | GTGTCACAAAGTAAGGATTCTGATGTGTATATCACAGACAAAACTGTGCTAGACATGAGGTCT | |
| | ATGGACTTCAAGAGCAACAGTGCTGTGGCCTGGAGCAACAAATCTGACTTTGCATGTGCAAAC | |
| | GCCTTCAACAACAGCATTATTCCAGAAGACACCTTCTTCCCCAGCCCAGAAAGTTCCTGTGAT | |
| | GTCAAGCTGGTCGAGAAAAGCTTTGAAACAGATACGAACCTAAACTTTCAAAACCTGTCAGTG | |
| | ATTGGGTTCCGAATCCTCCTCCTGAAAGTGGCCGGGTTTAATCTGCTCATGACGCTGCGGCTG | |
| | TGGTCCAGCGGCTCCGGAGCCACGAACTTCTCTCTGTTAAAGCAAGCAGGAGACGTGGAAGAA | |
| | AACCCCGGTCCCATGGGCCCTGGGCTCCTCTGCTGGGTGCTGCTTTGTCTCCTGGGAGCAGGC | |
| | CCAGTGGACGCTGGAGTCACCCAAAGTCCCACACACCTGATCAAAACGAGAGGACAGCACGTG | |
| | ACTCTGAGATGCTCTCCTATCTCTGGGCACAAGAGTGTGTCCTGGTACCAACAGGTCCTGGGT | |
| | CAGGGGCCCCAGTTTATCTTTCAGTATTATGAGAAAGAAGAGAGAGGAAGAGGAAACTTCCCT | |
| | GATCGATTCTCAGCTCGCCAGTTCCCTAACTATAGCTCTGAGCTGAATGTGAACGCCTTGTTG | |
| | CTGGGGGACTCGGCCCTGTATCTCTGTGCCAGCAGCGCCGACAGGGGGTCACCCCTCCACTTT | |
| | GGGAACGGGACCAGGCTCACTGTGACAGAGGACCTGAACAAGGTGTTCCCACCCGAGGTCGCT | |
| | GTGTTTGAGCCATCAGAAGCAGAGATCTCCCACACCCAAAAGGCCACACTGGTGTGCCTGGCC | |
| | ACAGGCTTCTTCCCCGACCACGTGGAGCTGAGCTGGTGGGTGAATGGGAAGGAGGTGCACAGT | |
| | GGGGTCAGCACGGACCCGCAGCCCCTCAAGGAGCAGCCCGCCCTCAATGACTCCAGATACTGC | |
| | CTGAGCAGCCGCCTGAGGGTCTCGGCCACCTTCTGGCAGAACCCCCGCAACCACTTCCGCTGT | |
| | CAAGTCCAGTTCTACGGGCTCTCGGAGAATGACGAGTGGACCCAGGATAGGGCCAAACCCGTC | |
| | ACCCAGATCGTCAGCGCCGAGGCCTGGGGTAGAGCAGACTGTGGCTTTACCTCGGTGTCCTAC | |
| | CAGCAAGGGGTCCTGTCTGCCACCATCCTCTATGAGATCCTGCTAGGGAAGGCCACCCTGTAT | |
| | GCTGTGCTGGTCAGCGCCCTTGTGTTGATGGCCATGGTCAAGAGAAAGGATTTCTGA | |
| | α chain nucleotides: 1-837 | |
| | P2A nucleotides: 838-894 | |
| | ß chain nucleotides: 895-1821 | |
| 490 | SGHKS | TCR AJ Vβ |
| | | CDR-1 |
| 491 | YYEKEE | TCR AJ Vβ |
| | | CDR-2 |

1. A pair of nucleic acid sequences encoding a T cell receptor (TCR) or antigen-binding fragment thereof that recognizes a minor histocompatibility antigen HA-1 peptide having the sequence VLHDDLLEA (SEQ ID NO:354), wherein the pair of nucleic acid sequences encode:
an alpha chain comprising a variable alpha (Vα) region and a beta chain comprising a variable beta (Vβ) region; or
a gamma chain comprising a variable gamma (Vγ) region and a delta chain comprising a variable delta (Vδ) region; wherein:
(a) the Vα or Vγ region comprises a complementarity determining region 3 (CDR-3) comprising SEQ ID NO:3, and the Vβ or Vδ region comprises a CDR-3 comprising SEQ ID NO:11;
(b) the Vα or Vγ region comprises a CDR-3 comprising SEQ ID NO:21, and the Vβ or Vδ region comprises a CDR-3 comprising SEQ ID NO:27;
(c) the Vα or Vγ region comprises a CDR-3 comprising SEQ ID NO:37, and the Vβ or Vδ region comprises a CDR-3 comprising SEQ ID NO:43;
(d) the Vα or Vγ region comprises a CDR-3 comprising SEQ ID NO:51, and the Vβ or Vδ region comprises a CDR-3 comprising SEQ ID NO:57;
(e) the Vα or Vγ region comprises a CDR-3 comprising SEQ ID NO:65, and the Vβ or Vδ region comprises a CDR-3 comprising SEQ ID NO:57;
(f) the Vα or Vγ region comprises a CDR-3 comprising SEQ ID NO:78, and the Vβ or Vδ region comprises a CDR-3 comprising SEQ ID NO:84;
(g) the Vα or Vγ region comprises a CDR-3 comprising SEQ ID NO:92, and the Vβ or Vδ region comprises a CDR-3 comprising SEQ ID NO:98;
(h) the Vα or Vγ region comprises a CDR-3 comprising SEQ ID NO: 106, and the Vβ or Vδ region comprises a CDR-3 comprising SEQ ID NO:112;
(i) the Vα or Vγ region comprises a CDR-3 comprising SEQ ID NO: 120, and the Vβ or Vδ region comprises a CDR-3 comprising SEQ ID NO:126;
(j) the Vα or Vγ region comprises a CDR-3 comprising SEQ ID NO:136, and the Vβ or Vδ region comprises a CDR-3 comprising SEQ ID NO:142;
(k) the Vα or Vγ region comprises a CDR-3 comprising SEQ ID NO: 152, and the Vβ or Vδ region comprises a CDR-3 comprising SEQ ID NO:158;
(1) the Vα or Vγ region comprises a CDR-3 comprising SEQ ID NO:166, and the Vβ or Vδ region comprises a CDR-3 comprising SEQ ID NO:172;
(m) the Vα or Vγ region comprises a CDR-3 comprising SEQ ID NO: 180, and the Vβ or Vδ region comprises a CDR-3 comprising SEQ ID NO:186;
(n) the Vα or Vγ region comprises a CDR-3 comprising SEQ ID NO: 194, and the Vβ or Vδ region comprises a CDR-3 comprising SEQ ID NO:200;
(o) the Vα or Vγ region comprises a CDR-3 comprising SEQ ID NO:208, and the Vβ or Vδ region comprises a CDR-3 comprising SEQ ID NO:214;
(p) the Vα or Vγ region comprises a CDR-3 comprising SEQ ID NO:224, and the Vβ or Vδ region comprises a CDR-3 comprising SEQ ID NO:230;
(q) the Vα or Vγ region comprises a CDR-3 comprising SEQ ID NO:238, and the Vβ or Vδ region comprises a CDR-3 comprising SEQ ID NO:244;
(r) the Vα or Vγ region comprises a CDR-3 comprising SEQ ID NO:252, and the Vβ or Vδ region comprises a CDR-3 comprising SEQ ID NO:258;
(s) the Vα or Vγ region comprises a CDR-3 comprising SEQ ID NO:268, and the Vβ or Vδ region comprises a CDR-3 comprising SEQ ID NO:158;
(t) the Vα or Vγ region comprises a CDR-3 comprising SEQ ID NO:278, and the Vβ or Vδ region comprises a CDR-3 comprising SEQ ID NO:284;
(u) the Vα or Vγ region comprises a CDR-3 comprising SEQ ID NO:359, and the Vβ or Vδ region comprises a CDR-3 comprising SEQ ID NO:363;
(v) the Vα or Vγ region comprises a CDR-3 comprising SEQ ID NO:369, and the Vβ or Vδ region comprises a CDR-3 comprising SEQ ID NO:373;
(w) the Vα or Vγ region comprises a CDR-3 comprising SEQ ID NO:379, and the Vβ or Vδ region comprises a CDR-3 comprising SEQ ID NO:383;
(x) the Vα or Vγ region comprises a CDR-3 comprising SEQ ID NO:389, and the Vβ or Vδ region comprises a CDR-3 comprising SEQ ID NO:393;
(y) the Vα or Vγ region comprises a CDR-3 comprising SEQ ID NO:399, and the Vβ or Vδ region comprises a CDR-3 comprising SEQ ID NO:403;
(z) the Vα or Vγ region comprises a CDR-3 comprising SEQ ID NO:409, and the Vβ or Vδ region comprises a CDR-3 comprising SEQ ID NO:413;
(aa) the Vα or Vγ region comprises a CDR-3 comprising SEQ ID NO:419, and the Vβ or Vδ region comprises a CDR-3 comprising SEQ ID NO:423;
(ab) the Vα or Vγ region comprises a CDR-3 comprising SEQ ID NO:429, and the Vβ or Vδ region comprises a CDR-3 comprising SEQ ID NO:433;
(ac) the Vα or Vγ region comprises a CDR-3 comprising SEQ ID NO:439, and the Vβ or Vδ region comprises a CDR-3 comprising SEQ ID NO:443;
(ad) the Vα or Vγ region comprises a CDR-3 comprising SEQ ID NO:449, and the Vβ or Vδ region comprises a CDR-3 comprising SEQ ID NO: 453;
(ae) the Vα or Vγ region comprises a CDR-3 comprising SEQ ID NO:480, and the Vβ or Vδ region comprises a CDR-3 comprising SEQ ID NO:484;
(af) the Vα or Vγ region comprises a complementarity determining region 3 (CDR-3) comprising SEQ ID NO:459, 460, 461, 462, 463, 464, 476, or 477, and the Vβ or Vo region comprises a CDR-3 comprising SEQ ID NO:465, 466, 467, 468, 469, 478, or 479;
(ag) the Vα or Vγ region comprises the CDR-3 comprising SEQ ID NO:463, and the Vβ or Vδ region comprises the CDR-3 comprising SEQ ID NO:469;
(ah) the Vα or Vγ region comprises a CDR-3 comprising SEQ ID NO:470, 471, 472, 473. and the Vβ or Vδ region comprises a CDR-3 comprising SEQ ID NO:57; or
(ai) the Vα or Vγ region comprises a CDR-3 comprising SEQ ID NO:474, and the Vβ or Vδ region comprises a CDR-3 comprising SEQ ID NO:475.

2. (canceled)

3. The pair of nucleic acid sequences of claim 1, wherein:
(a) the Vα or Vγ region comprises a complementarity determining region 1 (CDR-1) comprising SEQ ID NO: 1, a complementarity determining region 2 (CDR-2) comprising SEQ ID NO:2, and a complementarity determining region 3 (CDR-3) comprising SEQ ID NO:3, and the Vβ or Vδ region comprises a CDR-1 comprising SEQ ID NO:9, a CDR-2 comprising SEQ ID NO:10, and a CDR-3 comprising SEQ ID NO:11;
(b) the Vα or Vγ region comprises a CDR-1 comprising SEQ ID NO: 19, a CDR-2 comprising SEQ ID NO:20, and a CDR-3 comprising SEQ ID NO:21, and the Vβ or Vδ region comprises a CDR-1 comprising SEQ ID NO:9, a CDR-2 comprising SEQ ID NO:10, and a CDR-3 comprising SEQ ID NO:27;
(c) the Vα or Vγ region comprises a CDR-1 comprising SEQ ID NO:35, a CDR-2 comprising SEQ ID NO:36, and a CDR-3 comprising SEQ ID NO:37, and the Vβ or Vδ region comprises a CDR-1 comprising SEQ ID NO:9, a CDR-2 comprising SEQ ID NO:10, and a CDR-3 comprising SEQ ID NO:43;
(d) the Vα or Vγ region comprises a CDR-1 comprising SEQ ID NO:1, a CDR-2 comprising SEQ ID NO:2, and a CDR-3 comprising SEQ ID NO:51, and the Vβ or Vδ region comprises a CDR-1 comprising SEQ ID NO:9, a CDR-2 comprising SEQ ID NO:10, and a CDR-3 comprising SEQ ID NO:57;
(e) the Vα or Vγ region comprises a CDR-1 comprising SEQ ID NO:1, a CDR-2 comprising SEQ ID NO:2, and a CDR-3 comprising SEQ ID NO:65, and the Vβ or Vδ region comprises a CDR-1 comprising SEQ ID NO:9, a CDR-2 comprising SEQ ID NO:10, and a CDR-3 comprising SEQ ID NO:57;
(f) the Vα or Vγ region comprises a CDR-1 comprising SEQ ID NO:76, a CDR-2 comprising SEQ ID NO:77, and a CDR-3 comprising SEQ ID NO:78, and the Vβ or Vδ region comprises a CDR-1 comprising SEQ ID NO:9, a CDR-2 comprising SEQ ID NO:10, and a CDR-3 comprising SEQ ID NO:84;
(g) the Vα or Vγ region comprises a CDR-1 comprising SEQ ID NO:19, a CDR-2 comprising SEQ ID NO:20, and a CDR-3 comprising SEQ ID NO:92, and the Vβ or Vδ region comprises a CDR-1 comprising SEQ ID NO:9, a CDR-2 comprising SEQ ID NO:10, and a CDR-3 comprising SEQ ID NO:98;
(h) the Vα or Vγ region comprises a CDR-1 comprising SEQ ID NO:76, a CDR-2 comprising SEQ ID NO:77, and a CDR-3 comprising SEQ ID NO:106, and the Vβ or Vδ region comprises a CDR-1 comprising SEQ ID NO:9, a CDR-2 comprising SEQ ID NO:10, and a CDR-3 comprising SEQ ID NO:112;
(i) the Vα or Vγ region comprises a CDR-1 comprising SEQ ID NO:35, a CDR-2 comprising SEQ ID NO:36, and a CDR-3 comprising SEQ ID NO: 120, and the Vβ or Vδ region comprises a CDR-1 comprising SEQ ID NO:9, a CDR-2 comprising SEQ ID NO:10, and a CDR-3 comprising SEQ ID NO:126;
(j) the Vα or Vγ region comprises a CDR-1 comprising SEQ ID NO:134, a CDR-2 comprising SEQ ID NO: 135, and a CDR-3 comprising SEQ ID NO: 136, and the Vβ or Vδ region comprises a CDR-1 comprising SEQ ID NO:9, a CDR-2 comprising SEQ ID NO:10, and a CDR-3 comprising SEQ ID NO:142;
(k) the Vα or Vγ region comprises a CDR-1 comprising SEQ ID NO:150, a CDR-2 comprising SEQ ID NO: 151, and a CDR-3 comprising SEQ ID NO: 152, and the Vβ or Vδ region comprises a CDR-1 comprising SEQ ID NO:9, a CDR-2 comprising SEQ ID NO:10, and a CDR-3 comprising SEQ ID NO:158;
(l) the Vα or Vγ region comprises a CDR-1 comprising SEQ ID NO:35, a CDR-2 comprising SEQ ID NO:36, and a CDR-3 comprising SEQ ID NO:166, and the Vβ or Vδ region comprises a CDR-1 comprising SEQ ID NO:9, a CDR-2 comprising SEQ ID NO:10, and a CDR-3 comprising SEQ ID NO:172;
(m) the Vα or Vγ region comprises a CDR-1 comprising SEQ ID NO:35, a CDR-2 comprising SEQ ID NO:36, and a CDR-3 comprising SEQ ID NO:180, and the Vβ or Vδ region comprises a CDR-1 comprising SEQ ID NO:9, a CDR-2 comprising SEQ ID NO:10, and a CDR-3 comprising SEQ ID NO:186;
(n) the Vα or Vγ region comprises a CDR-1 comprising SEQ ID NO:76, a CDR-2 comprising SEQ ID NO: 77, and a CDR-3 comprising SEQ ID NO: 194, and the Vβ or Vδ region comprises a CDR-1 comprising SEQ ID NO:9, a CDR-2 comprising SEQ ID NO:10, and a CDR-3 comprising SEQ ID NO:200;
(o) the Vα or Vγ region comprises a CDR-1 comprising SEQ ID NO:76, a CDR-2 comprising SEQ ID NO:77, and a CDR-3 comprising SEQ ID NO:208, and the Vβ or Vδ region comprises a CDR-1 comprising SEQ ID NO:9, a CDR-2 comprising SEQ ID NO:10, and a CDR-3 comprising SEQ ID NO:214;
(p) the Vα or Vγ region comprises a CDR-1 comprising SEQ ID NO:222, a CDR-2 comprising SEQ ID NO:223, and a CDR-3 comprising SEQ ID NO:224, and the Vβ or Vδ region comprises a CDR-1 comprising SEQ ID NO:9, a CDR-2 comprising SEQ ID NO:10, and a CDR-3 comprising SEQ ID NO:230;
(q) the Vα or Vγ region comprises a CDR-1 comprising SEQ ID NO:76, a CDR-2 comprising SEQ ID NO:77, and a CDR-3 comprising SEQ ID NO:238, and the Vβ or Vδ region comprises a CDR-1 comprising SEQ ID NO:9, a CDR-2 comprising SEQ ID NO:10, and a CDR-3 comprising SEQ ID NO:244;
(r) the Vα or Vγ region comprises a CDR-1 comprising SEQ ID NO:19, a CDR-2 comprising SEQ ID NO:20, and a CDR-3 comprising SEQ ID NO:252, and the Vβ or Vδ region comprises a CDR-1 comprising SEQ ID NO:9, a CDR-2 comprising SEQ ID NO:10, and a CDR-3 comprising SEQ ID NO:258;
(s) the Vα or Vγ region comprises a CDR-1 comprising SEQ ID NO:266, a CDR-2 comprising SEQ ID NO:267, and a CDR-3 comprising SEQ ID NO:268, and the Vβ or Vδ region comprises a CDR-1 comprising SEQ ID NO:9, a CDR-2 comprising SEQ ID NO:10, and a CDR-3 comprising SEQ ID NO: 158; (t) the Vα or Vγ region comprises a CDR-1 comprising SEQ ID NO: 19, a CDR-2 comprising SEQ ID NO:20, and a CDR-3 comprising SEQ ID NO:278, and the Vβ or Vδ region comprises a CDR-1 comprising SEQ ID NO:9, a CDR-2 comprising SEQ ID NO:10, and a CDR-3 comprising SEQ ID NO:284;
(u) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:360, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:364;
(v) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:370, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:374;
(w) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:380, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:384;
(x) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:390, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:394;
(y) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:400, and the Vβ or Vo region comprises a CDR-1, a CDR-2, and a CDR-3 comprising a CDR-1, a CDR-2, and a CDR-3, respectively, contained within the Vβ or Vδ region sequence of SEQ ID NO:404;
(z) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:410, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:414;
(aa) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:420, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:424;
(ab) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:430, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:434;
(ac) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:440, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:444;
(ad) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:450, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:454;
(ae) the Vα or Vγ region comprises a CDR-1, a CDR-2, and a CDR-3 comprising a CDR-1, a CDR-2, and a CDR-3, respectively, contained within the Vα or Vγ region sequence of SEQ ID NO:481, and the Vβ or Vδ region comprises a CDR-1, a CDR-2, and a CDR-3 comprising a CDR-1, a CDR-2, and a CDR-3, respectively, contained within the Vβ or Vδ region sequence of SEQ ID NO:485; or
(af) the Vα or Vγ region comprises
(i) a CDR-1 comprising SEQ ID NO:1, and a CDR-2 comprising SEQ ID NO:2;
(ii) a CDR-1 comprising SEQ ID NO:19, and a CDR-2 comprising SEQ ID NO:20;
(iii) a CDR-1 comprising SEQ ID NO:35, and a CDR-2 comprising SEQ ID NO:36;
(iv) a CDR-1 comprising SEQ ID NO:76, and a CDR-2 comprising SEQ ID NO:77;
(v) a CDR-1 comprising SEQ ID NO: 134, and a CDR-2 comprising SEQ ID NO:135;
(vi) a CDR-1 comprising SEQ ID NO:150, and a CDR-2 comprising SEQ ID NO: 151;
(vii) a CDR-1 comprising SEQ ID NO:222, and a CDR-2 comprising SEQ ID NO:223; or
(viii) a CDR-1 comprising SEQ ID NO:266, and a CDR-2 comprising SEQ ID NO:267; and
a CDR3 SEQ ID NO:459, 460, 461, 462, 463, 464, 476, or 477, and the the Vβ or Vδ region comprises a CDR-1 comprising SEQ ID NO:9, and a CDR-2 comprising SEQ ID NO: 10 and a CDR3 comprising SEQ ID NO:465, 466, 467, 468, 469, 478, or 479.

4-6. (canceled)

7. The pair of nucleic acid sequences of claim 1, wherein:
(a) the Vα or Vγ region comprises SEQ ID NO:4 or a sequence that has at least 90% sequence identity thereto, and the Vβ or Vδ region comprises SEQ ID NO: 12 or a sequence that has at least 90% sequence identity thereto;
(b) the Vα or Vγ region comprises SEQ ID NO:22 or a sequence that has at least 90% sequence identity thereto, and the Vβ or Vδ region comprises SEQ ID NO:28 or a sequence that has at least 90% sequence identity thereto;
(c) the Vα or Vγ region comprises SEQ ID NO:38 or a sequence that has at least 90% sequence identity thereto, and the Vβ or Vδ region comprises SEQ ID NO:44 or a sequence that has at least 90% sequence identity thereto;
(d) the Vα or Vγ region comprises SEQ ID NO:52 or a sequence that has at least 90% sequence identity thereto, and the Vβ or Vδ region comprises SEQ ID NO:58 or a sequence that has at least 90% sequence identity thereto;
(e) the Vα or Vγ region comprises SEQ ID NO:66 or a sequence that has at least 90% sequence identity thereto, and the Vβ or Vδ region comprises SEQ ID NO:58 or a sequence that has at least 90% sequence identity thereto;
(f) the Vα or Vγ region comprises SEQ ID NO:79 or a sequence that has at least 90% sequence identity thereto, and the Vβ or Vδ region comprises SEQ ID NO:85 or a sequence that has at least 90% sequence identity thereto;
(g) the Vα or Vγ region comprises SEQ ID NO:93 or a sequence that has at least 90% sequence identity thereto, and the Vβ or Vδ region comprises SEQ ID NO:99 or a sequence that has at least 90% sequence identity thereto;
(h) the Vα or Vγ region comprises SEQ ID NO: 107 or a sequence that has at least 90% sequence identity thereto, and the Vβ or Vδ region comprises SEQ ID NO: 113 or a sequence that has at least 90% sequence identity thereto;
(i) the Vα or Vγ region comprises SEQ ID NO: 121 or a sequence that has at least 90% sequence identity thereto, and the Vβ or Vδ region comprises SEQ ID NO: 127 or a sequence that has at least 90% sequence identity thereto;
(j) the Vα or Vγ region comprises SEQ ID NO: 137 or a sequence that has at least 90% sequence identity thereto, and the Vβ or Vδ region comprises SEQ ID NO: 143 or a sequence that has at least 90% sequence identity thereto;
(k) the Vα or Vγ region comprises SEQ ID NO: 153 or a sequence that has at least 90% sequence identity thereto, and the Vβ or Vδ region comprises SEQ ID NO: 159 or a sequence that has at least 90% sequence identity thereto;
(l) the Vα or Vγ region comprises SEQ ID NO: 167 or a sequence that has at least 90% sequence identity thereto, and the Vβ or Vδ region comprises SEQ ID NO: 173 or a sequence that has at least 90% sequence identity thereto;
(m) the Vα or Vγ region comprises SEQ ID NO: 181 or a sequence that has at least 90% sequence identity thereto, and the Vβ or Vδ region comprises SEQ ID NO: 187 or a sequence that has at least 90% sequence identity thereto;
(n) the Vα or Vγ region comprises SEQ ID NO: 195 or a sequence that has at least 90% sequence identity thereto, and the Vβ or Vδ region comprises SEQ ID NO:201 or a sequence that has at least 90% sequence identity thereto;
(o) the Vα or Vγ region comprises SEQ ID NO:209 or a sequence that has at least 90% sequence identity thereto, and the Vβ or Vδ region comprises SEQ ID NO:215 or a sequence that has at least 90% sequence identity thereto;
(p) the Vα or Vγ region comprises SEQ ID NO:225 or a sequence that has at least 90% sequence identity thereto, and the Vβ or Vδ region comprises SEQ ID NO:231 or a sequence that has at least 90% sequence identity thereto;
(q) the Vα or Vγ region comprises SEQ ID NO:239 or a sequence that has at least 90% sequence identity thereto, and the Vβ or Vδ region comprises SEQ ID NO:245 or a sequence that has at least 90% sequence identity thereto;
(r) the Vα or Vγ region comprises SEQ ID NO:253 or a sequence that has at least 90% sequence identity thereto, and the Vβ or Vδ region comprises SEQ ID NO:259 or a sequence that has at least 90% sequence identity thereto;
(s) the Vα or Vγ region comprises SEQ ID NO:269 or a sequence that has at least 90% sequence identity thereto, and the Vβ or Vδ region comprises SEQ ID NO: 159 or a sequence that has at least 90% sequence identity thereto;
(t) the Vα or Vγ region comprises SEQ ID NO:279 or a sequence that has at least 90% sequence identity thereto, and the Vβ or Vδ region comprises SEQ ID NO:285 or a sequence that has at least 90% sequence identity thereto;
(u) the Vα or Vγ region comprises SEQ ID NO:360 or a sequence that has at least 90% sequence identity thereto, and the Vβ or Vδ region comprises SEQ ID NO:364 or a sequence that has at least 90% sequence identity thereto;
(v) the Vα or Vγ region comprises SEQ ID NO:370 or a sequence that has at least 90% sequence identity thereto, and the Vβ or Vδ region comprises SEQ ID NO:374 or a sequence that has at least 90% sequence identity thereto;
(w) the Vα or Vγ region comprises SEQ ID NO:380 or a sequence that has at least 90% sequence identity thereto, and the Vβ or Vδ region comprises SEQ ID NO:384 or a sequence that has at least 90% sequence identity thereto;
(x) the Vα or Vγ region comprises SEQ ID NO:390 or a sequence that has at least 90% sequence identity thereto, and the Vβ or Vδ region comprises SEQ ID NO:394 or a sequence that has at least 90% sequence identity thereto;
(y) the Vα or Vγ region comprises SEQ ID NO:400 or a sequence that has at least 90% sequence identity thereto, and the Vβ or Vδ region comprises SEQ ID NO:404 or a sequence that has at least 90% sequence identity thereto;
(z) the Vα or Vγ region comprises SEQ ID NO:410 or a sequence that has at least 90% sequence identity thereto, and the Vβ or Vδ region comprises SEQ ID NO:414 or a sequence that has at least 90% sequence identity thereto;
(aa) the Vα or Vγ region comprises SEQ ID NO:420 or a sequence that has at least 90% sequence identity thereto, and the Vβ or Vδ region comprises SEQ ID NO:424 or a sequence that has at least 90% sequence identity thereto;
(ab) the Vα or Vγ region comprises SEQ ID NO:430 or a sequence that has at least 90% sequence identity thereto, and the Vβ or Vδ region comprises SEQ ID NO:434 or a sequence that has at least 90% sequence identity thereto;
(ac) the Vα or Vγ region comprises SEQ ID NO:440 or a sequence that has at least 90% sequence identity thereto, and the Vβ or Vδ region comprises SEQ ID NO:444 or a sequence that has at least 90% sequence identity thereto;
(ad) the Vα or Vγ region comprises SEQ ID NO:450 or a sequence that has at least 90% sequence identity thereto, and the Vβ or Vδ region comprises SEQ ID NO:454 or a sequence that has at least 90% sequence identity thereto; or
(ae) the Vα or Vγ region comprises SEQ ID NO:481 or a sequence that has at least 90% sequence identity thereto, and the Vβ or Vδ region comprises SEQ ID NO:485 or a sequence that has at least 90% sequence identity thereto.

8-12. (canceled)

13. The pair of nucleic acid sequences of claim 1, wherein:
the alpha chain further comprises an alpha constant (Cα) region and the beta chain further comprises a beta constant (Cβ) region; or
the gamma chain further comprises an gamma constant (Cγ) region and the delta chain further comprises a delta constant (Cδ) region.

14. The pair of nucleic acid sequences of claim 13, wherein the Cα comprises SEQ ID NO: 294 or 296 and the Cβ comprises SEQ ID NO: 297 or 299.

15. The pair of nucleic acid sequences of claim 14, wherein:
(a) the alpha or gamma chain comprises SEQ ID NO:6, and the beta or delta chain comprises SEQ ID NO:14;
(b) the alpha or gamma chain comprises SEQ ID NO:24, and the beta or delta chain comprises SEQ ID NO:30;
(c) the alpha or gamma chain comprises SEQ ID NO:40, and the beta or delta chain comprises SEQ ID NO:46;
(d) the alpha or gamma chain comprises SEQ ID NO:54, and the beta or delta chain comprises SEQ ID NO:60;
(e) the alpha or gamma chain comprises SEQ ID NO:68, and the beta or delta chain comprises SEQ ID NO:71;
(f) the alpha or gamma chain comprises SEQ ID NO:81, and the beta or delta chain comprises SEQ ID NO:87;
(g) the alpha or gamma chain comprises SEQ ID NO:95, and the beta or delta chain comprises SEQ ID NO:101;
(h) the alpha or gamma chain comprises SEQ ID NO:109, and the beta or delta chain comprises SEQ ID NO:115;
(i) the alpha or gamma chain comprises SEQ ID NO:123, and the beta or delta chain comprises SEQ ID NO:129;
(j) the alpha or gamma chain comprises SEQ ID NO:139, and the beta or delta chain comprises SEQ ID NO:145;
(k) the alpha or gamma chain comprises SEQ ID NO:155, and the beta or delta chain comprises SEQ ID NO:161;
(l) the alpha or gamma chain comprises SEQ ID NO:169, and the beta or delta chain comprises SEQ ID NO:175;
(m) the alpha or gamma chain comprises SEQ ID NO:183, and the beta or delta chain comprises SEQ ID NO:189;
(n) the alpha or gamma chain comprises SEQ ID NO:197, and the beta or delta chain comprises SEQ ID NO:203;
(o) the alpha or gamma chain comprises SEQ ID NO:211, and the beta or delta chain comprises SEQ ID NO:217;
(p) the alpha or gamma chain comprises SEQ ID NO:227, and the beta or delta chain comprises SEQ ID NO:233;
(q) the alpha or gamma chain comprises SEQ ID NO:241, and the beta or delta chain comprises SEQ ID NO:247;
(r) the alpha or gamma chain comprises SEQ ID NO:255, and the beta or delta chain comprises SEQ ID NO:261;
(s) the alpha or gamma chain comprises SEQ ID NO:271, and the beta or delta chain comprises SEQ ID NO:161;
(t) the alpha or gamma chain comprises SEQ ID NO:281, and the beta or delta chain comprises SEQ ID NO:287;
(u) the alpha or gamma chain comprises SEQ ID NO:362, and the beta or delta chain comprises SEQ ID NO:366;
(v) the alpha or gamma chain comprises SEQ ID NO:372, and the beta or delta chain comprises SEQ ID NO:376;
(w) the alpha or gamma chain comprises SEQ ID NO:382, and the beta or delta chain comprises SEQ ID NO:386;
(x) the alpha or gamma chain comprises SEQ ID NO:392, and the beta or delta chain comprises SEQ ID NO:396;
(y) the alpha or gamma chain comprises SEQ ID NO:402, and the beta or delta chain comprises SEQ ID NO:406;
(z) the alpha or gamma chain comprises SEQ ID NO:412, and the beta or delta chain comprises SEQ ID NO:416;
(aa) the alpha or gamma chain comprises SEQ ID NO:422, and the beta or delta chain comprises SEQ ID NO:426;
(ab) the alpha or gamma chain comprises SEQ ID NO:432, and the beta or delta chain comprises SEQ ID NO:436;
(ac) the alpha or gamma chain comprises SEQ ID NO:442, and the beta or delta chain comprises SEQ ID NO:446;
(ad) the alpha or gamma chain comprises SEQ ID NO:452, and the beta or delta chain comprises SEQ ID NO:456; or
(ae) the alpha or gamma chain comprises SEQ ID NO:483, and the beta or delta chain comprises SEQ ID NO:487.

16. The pair of nucleic acid sequences of claim 1, wherein the encoded TCR or antigen-binding fragment thereof recognizes HA-1 in the context of a human leukocyte antigens (HLA)-A molecule of serotype HLA-A*02:01 or HLA-A*02:06.

17-20. (canceled)

21. The pair of nucleic acid sequences of claim 1, wherein the TCR or antigen-binding fragment thereof recognizes the peptide having the sequence VLHDDLLEA (SEQ ID NO:354) with higher affinity than a peptide having the sequence VLRDDLLEA (SEQ ID NO:355).

22. (canceled)

23. The pair of nucleic acid sequences of claim 1, wherein the nucleic acid comprises:
(a) a nucleotide sequence encoding the Vα or Vγ region comprises SEQ ID NO:7 or a sequence that has at least 90% sequence identity thereto, and a nucleotide sequence encoding the Vβ or Vδ region comprises SEQ ID NO: 15 or a sequence that has at least 90% sequence identity thereto;
(b) a nucleotide sequence encoding the Vα or Vγ region comprises SEQ ID NO:25 or a sequence that has at least 90% sequence identity thereto, and a nucleotide sequence encoding the Vβ or Vδ region comprises SEQ ID NO:31 or a sequence that has at least 90% sequence identity thereto;
(c) a nucleotide sequence encoding the Vα or Vγ region comprises SEQ ID NO:41 or a sequence that has at least 90% sequence identity thereto, and a nucleotide sequence encoding the Vβ or Vδ region comprises SEQ ID NO:47 or a sequence that has at least 90% sequence identity thereto;
(d) a nucleotide sequence encoding the Vα or Vγ region comprises SEQ ID NO:55 or a sequence that has at least 90% sequence identity thereto, and a nucleotide sequence encoding the Vβ or Vδ region comprises SEQ ID NO:61 or a sequence that has at least 90% sequence identity thereto;
(e) a nucleotide sequence encoding the Vα or Vγ region comprises SEQ ID NO:69 or a sequence that has at least 90% sequence identity thereto, and a nucleotide sequence encoding the Vβ or Vδ region comprises SEQ ID NO:72 or a sequence that has at least 90% sequence identity thereto;
(f) a nucleotide sequence encoding the Vα or Vγ region comprises SEQ ID NO:82 or a sequence that has at least 90% sequence identity thereto, and a nucleotide sequence encoding the Vβ or Vδ region comprises SEQ ID NO:88 or a sequence that has at least 90% sequence identity thereto;
(g) a nucleotide sequence encoding the Vα or Vγ region comprises SEQ ID NO:96 or a sequence that has at least 90% sequence identity thereto, and a nucleotide sequence encoding the Vβ or Vδ region comprises SEQ ID NO: 102 or a sequence that has at least 90% sequence identity thereto;
(h) a nucleotide sequence encoding the Vα or Vγ region comprises SEQ ID NO:110 or a sequence that has at least 90% sequence identity thereto, and a nucleotide sequence encoding the Vβ or Vδ region comprises SEQ ID NO: 116 or a sequence that has at least 90% sequence identity thereto;
(i) a nucleotide sequence encoding the Vα or Vγ region comprises SEQ ID NO:124 or a sequence that has at least 90% sequence identity thereto, and a nucleotide sequence encoding the Vβ or Vδ region comprises SEQ ID NO:130 or a sequence that has at least 90% sequence identity thereto;
(j) a nucleotide sequence encoding the Vα or Vγ region comprises SEQ ID NO:140 or a sequence that has at least 90% sequence identity thereto, and a nucleotide sequence encoding the Vβ or Vδ region comprises SEQ ID NO: 146 or a sequence that has at least 90% sequence identity thereto;
(k) a nucleotide sequence encoding the Vα or Vγ region comprises SEQ ID NO:156 or a sequence that has at least 90% sequence identity thereto, and a nucleotide sequence encoding the Vβ or Vδ region comprises SEQ ID NO: 162 or a sequence that has at least 90% sequence identity thereto;
(l) a nucleotide sequence encoding the Vα or Vγ region comprises SEQ ID NO:170 or a sequence that has at least 90% sequence identity thereto, and a nucleotide sequence encoding the Vβ or Vδ region comprises SEQ ID NO: 176 or a sequence that has at least 90% sequence identity thereto;
(m) a nucleotide sequence encoding the Vα or Vγ region comprises SEQ ID NO:184 or a sequence that has at least 90% sequence identity thereto, and a nucleotide sequence encoding the Vβ or Vδ region comprises SEQ ID NO:190 or a sequence that has at least 90% sequence identity thereto;
(n) a nucleotide sequence encoding the Vα or Vγ region comprises SEQ ID NO:198 or a sequence that has at least 90% sequence identity thereto, and a nucleotide sequence encoding the Vβ or Vδ region comprises SEQ ID NO:204 or a sequence that has at least 90% sequence identity thereto;
(o) a nucleotide sequence encoding the Vα or Vγ region comprises SEQ ID NO:212 or a sequence that has at least 90% sequence identity thereto, and a nucleotide sequence encoding the Vβ or Vδ region comprises SEQ ID NO:218 or a sequence that has at least 90% sequence identity thereto;
(p) a nucleotide sequence encoding the Vα or Vγ region comprises SEQ ID NO:228 or a sequence that has at least 90% sequence identity thereto, and a nucleotide sequence encoding the Vβ or Vδ region comprises SEQ ID NO:234 or a sequence that has at least 90% sequence identity thereto;
(q) a nucleotide sequence encoding the Vα or Vγ region comprises SEQ ID NO:242 or a sequence that has at least 90% sequence identity thereto, and a nucleotide sequence encoding the Vβ or Vδ region comprises SEQ ID NO:248 or a sequence that has at least 90% sequence identity thereto;
(r) a nucleotide sequence encoding the Vα or Vγ region comprises SEQ ID NO:256 or a sequence that has at least 90% sequence identity thereto, and a nucleotide sequence encoding the Vβ or Vδ region comprises SEQ ID NO:262 or a sequence that has at least 90% sequence identity thereto;
(s) a nucleotide sequence encoding the Vα or Vγ region comprises SEQ ID NO:272 or a sequence that has at least 90% sequence identity thereto, and a nucleotide sequence encoding the Vβ or Vδ region comprises SEQ ID NO:274 or a sequence that has at least 90% sequence identity thereto;
(t) a nucleotide sequence encoding the Vα or Vγ region comprises SEQ ID NO:282 or a sequence that has at least 90% sequence identity thereto, and a nucleotide sequence encoding the Vβ or Vδ region comprises SEQ ID NO:288 or a sequence that has at least 90% sequence identity thereto;
(u) a nucleotide sequence encoding the Vα or Vγ region comprises SEQ ID NO:301, and a nucleotide sequence encoding the Vβ or Vδ region comprises SEQ ID NO:321;
(v) a nucleotide sequence encoding the Vα or Vγ region comprises SEQ ID NO:302, and a nucleotide sequence encoding the Vβ or Vδ region comprises SEQ ID NO:322;
(w) a nucleotide sequence encoding the Vα or Vγ region comprises SEQ ID NO:303, and a nucleotide sequence encoding the Vβ or Vδ region comprises SEQ ID NO:323;
(x) a nucleotide sequence encoding the Vα or Vγ region comprises SEQ ID NO:304, and a nucleotide sequence encoding the Vβ or Vδ region comprises SEQ ID NO:324;
(y) a nucleotide sequence encoding the Vα or Vγ region comprises SEQ ID NO:305, and a nucleotide sequence encoding the Vβ or Vδ region comprises SEQ ID NO:325;
(z) a nucleotide sequence encoding the Vα or Vγ region comprises SEQ ID NO:306, and a nucleotide sequence encoding the Vβ or Vδ region comprises SEQ ID NO:326;
(aa) a nucleotide sequence encoding the Vα or Vγ region comprises SEQ ID NO:307, and a nucleotide sequence encoding the Vβ or Vo region comprises SEQ ID NO:327;
(ab) a nucleotide sequence encoding the Vα or Vγ region comprises SEQ ID NO:308, and a nucleotide sequence encoding the Vβ or Vδ region comprises SEQ ID NO:328;
(ac) a nucleotide sequence encoding the Vα or Vγ region comprises SEQ ID NO:309, and a nucleotide sequence encoding the Vβ or Vδ region comprises SEQ ID NO:329;
(ad) a nucleotide sequence encoding the Vα or Vγ region comprises SEQ ID NO:310, and a nucleotide sequence encoding the Vβ or Vδ region comprises SEQ ID NO:330;
(ae) a nucleotide sequence encoding the Vα or Vγ region comprises SEQ ID NO:311, and a nucleotide sequence encoding the Vβ or Vδ region comprises SEQ ID NO:331;
(af) a nucleotide sequence encoding the Vα or Vγ region comprises SEQ ID NO:312, and a nucleotide sequence encoding the Vβ or Vδ region comprises SEQ ID NO:332;
(ag) a nucleotide sequence encoding the Vα or Vγ region comprises SEQ ID NO:313, and a nucleotide sequence encoding the Vβ or Vδ region comprises SEQ ID NO:333;
(ah) a nucleotide sequence encoding the Vα or Vγ region comprises SEQ ID NO:314, and a nucleotide sequence encoding the Vβ or Vδ region comprises SEQ ID NO:334;
(ai) a nucleotide sequence encoding the Vα or Vγ region comprises SEQ ID NO:315, and a nucleotide sequence encoding the Vβ or Vo region comprises SEQ ID NO:335;
(aj) a nucleotide sequence encoding the Vα or Vγ region comprises SEQ ID NO:316, and a nucleotide sequence encoding the Vβ or Vδ region comprises SEQ ID NO:336;
(ak) a nucleotide sequence encoding the Vα or Vγ region comprises SEQ ID NO:317, and a nucleotide sequence encoding the Vβ or Vδ region comprises SEQ ID NO:337;
(al) a nucleotide sequence encoding the Vα or Vγ region comprises SEQ ID NO:318, and a nucleotide sequence encoding the Vβ or Vδ region comprises SEQ ID NO:338;
(am) a nucleotide sequence encoding the Vα or Vγ region comprises SEQ ID NO:319, and a nucleotide sequence encoding the Vβ or Vδ region comprises SEQ ID NO:339; or
(an) a nucleotide sequence encoding the Vα or Vγ region comprises SEQ ID NO:320, and a nucleotide sequence encoding the Vβ or Vδ region comprises SEQ ID NO:340.

24. (canceled)

25. (canceled)

26. The pair of nucleic acid sequences of claim 1, wherein the pair of nucleic acid sequences comprise:
(a) a nucleotide sequence encoding the alpha or gamma chain comprises SEQ ID NO:8 or a sequence that has at least 90% sequence identity thereto, and a nucleotide sequence encoding the beta or delta chain comprises SEQ ID NO: 16 or a sequence that has at least 90% sequence identity thereto;
(b) a nucleotide sequence encoding the alpha or gamma chain comprises SEQ ID NO:26 or a sequence that has at least 90% sequence identity thereto, and a nucleotide sequence encoding the beta or delta chain comprises SEQ ID NO:32 or a sequence that has at least 90% sequence identity thereto;
(c) a nucleotide sequence encoding the alpha or gamma chain comprises SEQ ID NO:42 or a sequence that has at least 90% sequence identity thereto, and a nucleotide sequence encoding the beta or delta chain comprises SEQ ID NO:48 or a sequence that has at least 90% sequence identity thereto;
(d) a nucleotide sequence encoding the alpha or gamma chain comprises SEQ ID NO:56 or a sequence that has at least 90% sequence identity thereto, and a nucleotide sequence encoding the beta or delta chain comprises SEQ ID NO:62 or a sequence that has at least 90% sequence identity thereto;
(e) a nucleotide sequence encoding the alpha or gamma chain comprises SEQ ID NO:70 or a sequence that has at least 90% sequence identity thereto, and a nucleotide sequence encoding the beta or delta chain comprises SEQ ID NO:73 or a sequence that has at least 90% sequence identity thereto;
(f) a nucleotide sequence encoding the alpha or gamma chain comprises SEQ ID NO:83 or a sequence that has at least 90% sequence identity thereto, and a nucleotide sequence encoding the beta or delta chain comprises SEQ ID NO:89 or a sequence that has at least 90% sequence identity thereto;
(g) a nucleotide sequence encoding the alpha or gamma chain comprises SEQ ID NO:97 or a sequence that has at least 90% sequence identity thereto, and a nucleotide sequence encoding the beta or delta chain comprises SEQ ID NO: 103 or a sequence that has at least 90% sequence identity thereto;
(h) a nucleotide sequence encoding the alpha or gamma chain comprises SEQ ID NO:111 or a sequence that has at least 90% sequence identity thereto, and a nucleotide sequence encoding the beta or delta chain comprises SEQ ID NO:117 or a sequence that has at least 90% sequence identity thereto;
(i) a nucleotide sequence encoding the alpha or gamma chain comprises SEQ ID NO:125 or a sequence that has at least 90% sequence identity thereto, and a nucleotide sequence encoding the beta or delta chain comprises SEQ ID NO: 131 or a sequence that has at least 90% sequence identity thereto;
(j) a nucleotide sequence encoding the alpha or gamma chain comprises SEQ ID NO:141 or a sequence that has at least 90% sequence identity thereto, and a nucleotide sequence encoding the beta or delta chain comprises SEQ ID NO: 147 or a sequence that has at least 90% sequence identity thereto;
(k) a nucleotide sequence encoding the alpha or gamma chain comprises SEQ ID NO:157 or a sequence that has at least 90% sequence identity thereto, and a nucleotide sequence encoding the beta or delta chain comprises SEQ ID NO: 163 or a sequence that has at least 90% sequence identity thereto;
(1) a nucleotide sequence encoding the alpha or gamma chain comprises SEQ ID NO:171 or a sequence that has at least 90% sequence identity thereto, and a nucleotide sequence encoding the beta or delta chain comprises SEQ ID NO:177 or a sequence that has at least 90% sequence identity thereto;
(m) a nucleotide sequence encoding the alpha or gamma chain comprises SEQ ID NO:185 or a sequence that has at least 90% sequence identity thereto, and a nucleotide sequence encoding the beta or delta chain comprises SEQ ID NO:191 or a sequence that has at least 90% sequence identity thereto;
(n) a nucleotide sequence encoding the alpha or gamma chain comprises SEQ ID NO: 199 or a sequence that has at least 90% sequence identity thereto, and a nucleotide sequence encoding the beta or delta chain comprises SEQ ID NO:205 or a sequence that has at least 90% sequence identity thereto;
(o) a nucleotide sequence encoding the alpha or gamma chain comprises SEQ ID NO:213 or a sequence that has at least 90% sequence identity thereto, and a nucleotide sequence encoding the beta or delta chain comprises SEQ ID NO:219 or a sequence that has at least 90% sequence identity thereto;
(p) a nucleotide sequence encoding the alpha or gamma chain comprises SEQ ID NO:229 or a sequence that has at least 90% sequence identity thereto, and a nucleotide sequence encoding the beta or delta chain comprises SEQ ID NO:235 or a sequence that has at least 90% sequence identity thereto;
(q) a nucleotide sequence encoding the alpha or gamma chain comprises SEQ ID NO:243 or a sequence that has at least 90% sequence identity thereto, and a nucleotide sequence encoding the beta or delta chain comprises SEQ ID NO:249 or a sequence that has at least 90% sequence identity thereto;
(r) a nucleotide sequence encoding the alpha or gamma chain comprises SEQ ID NO:257 or a sequence that has at least 90% sequence identity thereto, and a nucleotide sequence encoding the beta or delta chain comprises SEQ ID NO:263 or a sequence that has at least 90% sequence identity thereto;
(s) a nucleotide sequence encoding the alpha or gamma chain comprises SEQ ID NO:273 or a sequence that has at least 90% sequence identity thereto, and a nucleotide sequence encoding the beta or delta chain comprises SEQ ID NO:275 or a sequence that has at least 90% sequence identity thereto; or
(t) a nucleotide sequence encoding the alpha or gamma chain comprises SEQ ID NO:283 or a sequence that has at least 90% sequence identity thereto, and a nucleotide sequence encoding the beta or delta chain comprises SEQ ID NO:289 or a sequence that has at least 90% sequence identity thereto.

27. (canceled)

28. The pair of nucleic acid sequences of claim 26, wherein the nucleotide sequence encoding the alpha chain and the nucleotide sequence encoding the beta chain are provided in a single open reading frame and are separated by a nucleotide sequence encoding a peptide sequence that causes ribosome skipping.

29. The pair of nucleic acid sequences of claim 28, wherein the peptide that causes ribosome skipping comprises a P2A peptide.

30. (canceled)

31. (canceled)

32. The pair of nucleic acid sequences of claim 29, wherein:
(a) the single open reading frame encodes SEQ ID NO: 18;
(b) the single open reading frame encodes SEQ ID NO:34;
(c) the single open reading frame encodes SEQ ID NO:50;
(d) the single open reading frame encodes SEQ ID NO:64;
(e) the single open reading frame encodes SEQ ID NO:75;
the single open reading frame encodes SEQ ID NO:91; (f)
(g) the single open reading frame encodes SEQ ID NO: 105;
(h) the single open reading frame encodes SEQ ID NO: 119;
(i) the single open reading frame encodes SEQ ID NO: 133;
(j) the single open reading frame encodes SEQ ID NO: 149;
(k) the single open reading frame encodes SEQ ID NO: 165;
(l) the single open reading frame encodes SEQ ID NO:179;
(m) the single open reading frame encodes SEQ ID NO: 193;
(n) the single open reading frame encodes SEQ ID NO:207;
(o) the single open reading frame encodes SEQ ID NO:221;
(p) the single open reading frame encodes SEQ ID NO:237;
(q) the single open reading frame encodes SEQ ID NO:251;
(r) the single open reading frame encodes SEQ ID NO:265;
(s) the single open reading frame encodes SEQ ID NO:277;
(t) the single open reading frame encodes SEQ ID NO:291;
(u) the single open reading frame encodes SEQ ID NO:367;
(v) the single open reading frame encodes SEQ ID NO:377;
(w) the single open reading frame encodes SEQ ID NO:387;
(x) the single open reading frame encodes SEQ ID NO:397;
(y) the single open reading frame encodes SEQ ID NO:407;
(z) the single open reading frame encodes SEQ ID NO:417;
(aa) the single open reading frame encodes SEQ ID NO:427;
(ab) the single open reading frame encodes SEQ ID NO:437;
(ac) the single open reading frame encodes SEQ ID NO:447;
(ad) the single open reading frame encodes SEQ ID NO:457; or
(ae) the single open reading frame encodes SEQ ID NO:488.

33. The pair of nucleic acid of claim 32, wherein the single open reading frame comprises:
(a) the nucleotide sequence of SEQ ID NO: 17;
(b) the nucleotide sequence of SEQ ID NO:33;
(c) the nucleotide sequence of SEQ ID NO:49;
(d) the nucleotide sequence of SEQ ID NO:63;
(e) the nucleotide sequence of SEQ ID NO: 74;
(f) the nucleotide sequence of SEQ ID NO:90;
(g) the nucleotide sequence of SEQ ID NO: 104;
(h) the nucleotide sequence of SEQ ID NO:118;
(i) the nucleotide sequence of SEQ ID NO: 132;
(j) the nucleotide sequence of SEQ ID NO:148;
(k) the nucleotide sequence of SEQ ID NO:164;
(l) the nucleotide sequence of SEQ ID NO:178;
(m) the nucleotide sequence of SEQ ID NO:192;
(n) the nucleotide sequence of SEQ ID NO:206;
(o) the nucleotide sequence of SEQ ID NO:220;
(p) the nucleotide sequence of SEQ ID NO:236;
(q) the nucleotide sequence of SEQ ID NO:250;
(r) the nucleotide sequence of SEQ ID NO:264;
(s) the nucleotide sequence of SEQ ID NO:276;
(t) the nucleotide sequence of SEQ ID NO:290;
(u) the nucleotide sequence of SEQ ID NO:368;
(v) the nucleotide sequence of SEQ ID NO:378;
(w) the nucleotide sequence of SEQ ID NO:388;
(x) the nucleotide sequence of SEQ ID NO:398;
(y) the nucleotide sequence of SEQ ID NO:408;
(z) the nucleotide sequence of SEQ ID NO:418;
(aa) the nucleotide sequence of SEQ ID NO:428;
(ab) the nucleotide sequence of SEQ ID NO:438;
(ac) the nucleotide sequence of SEQ ID NO:448;
(ad) the nucleotide sequence of SEQ ID NO:458; or
(ae) the nucleotide sequence of SEQ ID NO:489.

34. A vector comprising the pair of nucleic acid sequences of claim 1.

35. (canceled)

36. (canceled)

37. An engineered cell, comprising the pair of nucleic acid sequences of claim 1.

38-41. (canceled)

42. The engineered cell of claim 37, wherein the engineered cell is a T cell.

43-65. (canceled)

66. A method of treating a malignant hematologic disorder comprising: administering to a subject a TCR encoded by the pair of nucleic acid sequences of claim 1 or an engineered T cell comprising the pair of nucleic acid sequences of claim 1.

67. The method of claim 66, wherein the subject is eligible for or is to receive an allogeneic hematopoietic stem cell transplantation.

68. (canceled)

69. The method of claim 66, wherein the subject has or has been diagnosed with acute myeloid leukemia (AML), myelodysplastic syndrome (MDS), or acute lymphoblastic leukemia (ALL).

70. The method of claim 66, wherein the subject has or has been diagnosed a liquid tumor, a hematopoietic tumor, a lymphoma, or chronic myeloid leukemia.

71. (canceled)
